(19) United States
(12) Patent Application Publication
(10) Pub. No.: US 2025/0320228 A1
(43) Pub. Date: Oct. 16, 2025

(54) PYRIDO[3,2-D]PYRIMIDINES AS HPK1 INHIBITORS

(71) Applicant: Merck Patent GmbH, Darmstadt (DE)

(72) Inventors: Momar TOURE, Billerica, MA (US); Yanping WANG, Billerica, MA (US); Constantin NEAGU, Henniker, NH (US); James CUMMINGS, Tewksbury, MA (US); Bin LI, Southborough, MA (US); Eugene CHEKLER, Concord, MA (US); Theresa JOHNSON, Salem, MA (US); Emily FRIIS, Somerville, MA (US)

(21) Appl. No.: 18/855,431

(22) PCT Filed: Apr. 6, 2023
(86) PCT No.: PCT/US2023/065444

§ 371 Date: Oct. 9, 2024

Related U.S. Application Data
(60) Provisional application No. 63/362,803, filed on Apr. 11, 2022.
(60) Provisional application No. 63/377,900, filed on Sep. 30, 2022.

(51) Int. Cl. C07D 519/00; A61K 31/519; A61K 31/5377; A61K 31/5386; A61K 31/551; C07D 471/04

(52) U.S. Cl. CPC C07D 519/00; A61K 31/519; A61K 31/5377; A61K 31/5386; A61K 31/551; C07D 471/04

(57) ABSTRACT

Pyrido[3,2-d]pyrimidine compounds are provided that are potent HPK1 inhibitors that are useful to treat or prevent cancer and/or inflammatory and/or autoimmune diseases or symptoms thereof in mammals, particularly humans. The compounds disclosed herein have a chemical structure of the general formula (I) and (II) or a prodrug or pharmaceutically acceptable salt of any of the foregoing including mixtures thereof in all ratios.

### CROSS-REFERENCE TO RELATED APPLICATIONS

The application claims the benefit of priority of US Provisional Application Nos. 63/377,900, filed Sep. 30, 2022 and 63/362,803, filed Apr. 11, 2022, the entire contents of which are incorporated herein by reference.

### BACKGROUND OF THE INVENTION

### Field of the Invention

Disclosed herein are pyrido[3,2-d]pyrimidine compounds that are potent HPK1 inhibitors that are useful to treat or prevent cancer and/or inflammatory and/or autoimmune diseases or symptoms thereof in mammals, particularly humans. The compounds disclosed herein have a chemical structure of the general formula (I) or (II) or a prodrug or pharmaceutically acceptable salt of any of the foregoing including mixtures thereof in all ratios.

### Description of Related Art

Hematopoietic progenitor kinase 1 (HPK1) is a serine/threonine kinase expressed in T cells, B-cells, and dendritic cells (Nature Immunology, 2006, vol. 8, pp. 84-91). In T cells, HPK1 acts as a rheostat of T cell activation by regulating the molecular circuits of the T cell receptor (TCR) signaling pathway. HPK1 is recruited to the TCR complex and phosphorylates SLP76 protein leading to its degradation and down-modulation of TCR signal strength. Genetic ablation of HPK1 results in T cell activation, lower TCR threshold, increased proliferation, and elevated levels of pro-inflammatory cytokines such as IL-2, TNF-α, and IFN-γ. Loss of HPK1 expression enhances dendritic cell activation and antigen presentation. (Hernandez S. et al., Cell Reports, 2018, vol. 25, pp. 80-94). HPK1 kinase activity is believed to be critical in conferring suppressive functions of HPK1 in a wide range of immune cells, such as CD8+, CD4+, DC, and NK to regulatory T cells (Tregs). Inactivation of the kinase domain of HPK1 was sufficient to elicit robust anti-tumor immune responses (Liu et al., PLoS One, Mar. 26, 2019). HPK1 knockout (KO) and kinase dead (KD) mice show enhanced T cell function and antitumor efficacy (You D. et. al., J. Immunother. Cancer 2021). Therefore, pharmacological inhibition of HPK1 has the potential to enhance effector T cell function and antitumor activity.

### SUMMARY OF THE INVENTION

In an embodiment, a compound of formula (I) is provided:

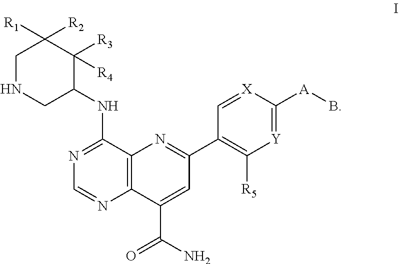

wherein:
R₁, R₂, R₃ and R₄ are each independently selected from the group consisting of H and halogen;
R₅ is selected from the group consisting of H, halogen, —O—C₁-C₆ alkyl, C₁-C₆ alkyl, C₁-C₆ haloalkyl and —CN;
X is selected from the group consisting of N and CR₆
Y is selected from the group consisting of N and CR₇
R₆ and R₇ are each independently selected from the group consisting of H, halogen and C₁-C₆ alkyl;
A is absent or selected from the group consisting of (—CH₂—)ₙ, —O—(—CH₂—)ₒ—, —O— and

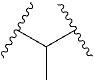 [SMILES: *C(*)C]

n is selected from the group consisting of 1, 2, 3 and 4;
o is selected from the group consisting of 1, 2, 3 and 4;
B is absent or selected from the group consisting of H, CN, halogen, optionally substituted C₆-C₁₄ aryl, optionally substituted C₂-C₁₄ heteroaryl, optionally substituted C₁-C₁₄ heterocyclic, optionally substituted C₁-C₈ alkyl, C₁-C₈ haloalkyl, optionally substituted C₁-C₈ alkyl interrupted by 1-4 heteroatoms, and optionally substituted C₃-C₁₄ cycloalkyl;
or a pharmaceutically acceptable salt, prodrugs, enantiomer, mixture of enantiomers, diastereomers or mixture of diastereomers

In another embodiment a compound having the formula Ia is provided.

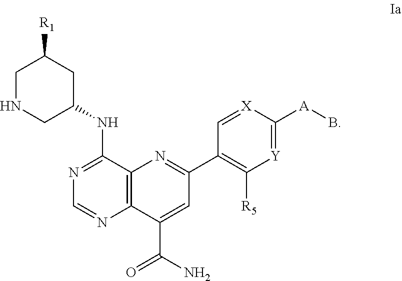

In another embodiment a compound having the formula Ib is provided.

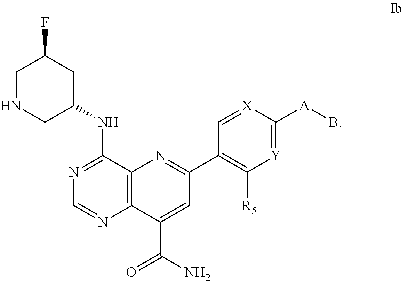

In another embodiment a compound having the formula Ic is provided.

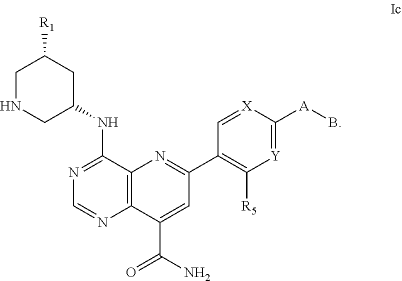

In another embodiment a compound having the formula Id is provided.

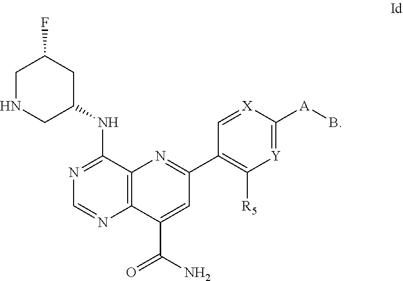

In another embodiment a compound having the formula Ie is provided.

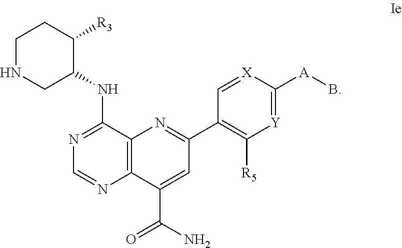

In another embodiment a compound having the formula If is provided.

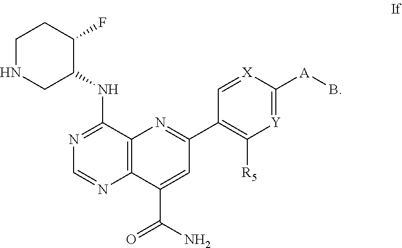

In another embodiment a compound having the formula Ig is provided.

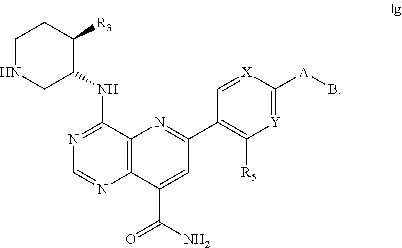

In another embodiment a compound having the formula Ih is provided.

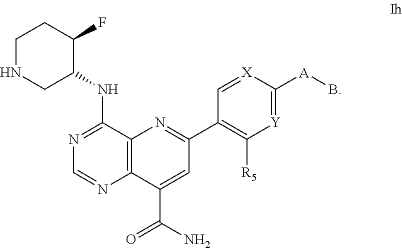

In another embodiment a compound having the formula Il is provided.

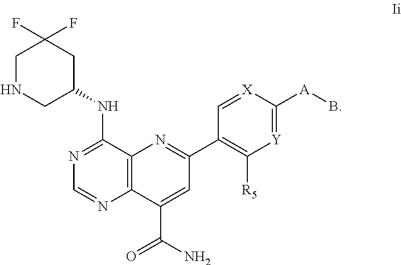

In another embodiment a compound having the formula Ij is provided.

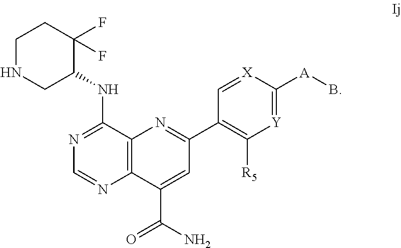

In another embodiment a compound having the formula Ik is provided.

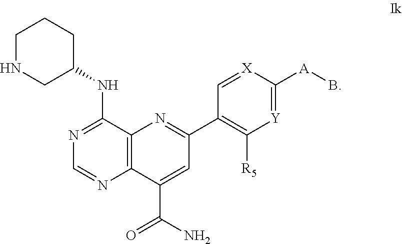

In an embodiment a compound of formula I wherein R₁, R₂, R₃ and R₄ are each H is provided.

In an embodiment a compound of formula I wherein R₁, R₃ and R₄ are H and R₂ is a halogen is provided.

In an embodiment a compound of formula I wherein R₂ is F is provided.

In an embodiment a compound of formula I wherein R₁, R₂, and R₃ are H and R₄ is halogen is provided.

In an embodiment a compound of formula I wherein R₄ is F is provided.

In an embodiment a compound of formula I wherein R₁ and R₂ are halogen and R₃ and R₄ are H is provided.

In an embodiment a compound of formula I wherein R₁ and R₂ are F is provided.

In an embodiment a compound of formula I wherein R₁ and R₂ are H and R₃ and R₄ are halogen is provided.

In an embodiment a compound of formula I wherein R₃ and R₄ are F are provided.

In an embodiment a compound of formula I wherein R₅ is selected from the group consisting of H, —CN and —CF₃ is provided.

In an embodiment a compound of formula I wherein R₅ is —CN is provided.

In an embodiment a compound of formula I wherein -A-B is selected from the group consisting of

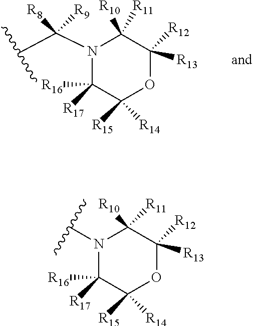

wherein R₈ and R₉ are each independently selected from the group consisting of H and C₁-C₆ alkyl; wherein R₈ and R₉ together with the carbon to which they are attached can form a ring having 3-6 carbon atoms; wherein R₁₀, R₁₁, R₁₂, R₁₃, R₁₄, R₁₅, R₁₆ and R₁₇ are each independently selected from the group consisting of H, C₁-C₆ alkyl, C₁-C₆ haloalkyl, halogen and —CN are provided. Preferably, R₈ and R₉ are H. Preferably, R₈ and R₉ together with the carbon they are attached form a cyclopropyl ring. Preferably, R₁₀, R₁₁, R₁₆ and R₁₇ are each independently selected from the group consisting of H and —CH₃; and R₁₂, R₁₃, R₁₄ and R₁₅ are H. Preferably, R₁₂ and R₁₃ are both F and R₁₀, R₁₁, R₁₄, R₁₅, R₁₆ and R₁₇ are H.

Preferably, R₁₀ and R₁₁ are each independently selected from the group consisting of H and —CN; and R₁₂, R₁₃, R₁₄, R₁₅, R₁₆ and R₁₇ are H. Preferably, R₁₀ and R₁₁ are each independently selected from the group consisting of H and —CF₃; and R₁₂, R₁₃, R₁₄, R₁₅, R₁₆ and R₁₇ are H. Preferably, R₁₂ and R₁₃ are each independently selected from the group consisting of H and —CN; and R₁₀, R₁₁, R₁₄, R₁₅, R₁₆ and R₁₇ are H. Preferably, R₁₂ and R₁₃ are each independently selected from the group consisting of H and —CF₃; and R₁₀, R₁₁, R₁₄, R₁₅, R₁₆ and R₁₇ are H. Preferably, R₈, R₉, R₁₀, R₁₁, R₁₂, R₁₃, R₁₄, R₁₅, R₁₆ and R₁₇ are H. Preferably, R₁₀, R₁₁, R₁₂, R₁₃, R₁₄, R₁₅, R₁₆ and R₁₇ are H. Preferably, R₁₂ and R₁₃ are each independently selected from the group consisting of H and —CH₃; and R₁₀, R₁₁, R₁₄, R₁₅, R₁₆ and R₁₇ are H is provided.

In an embodiment a compound of formula I wherein -A-B is selected from the group consisting of

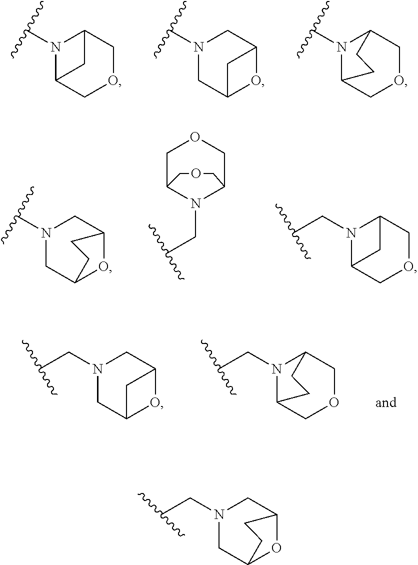

provided.

In an embodiment a compound of formula I wherein -A-B is selected from the group consisting of

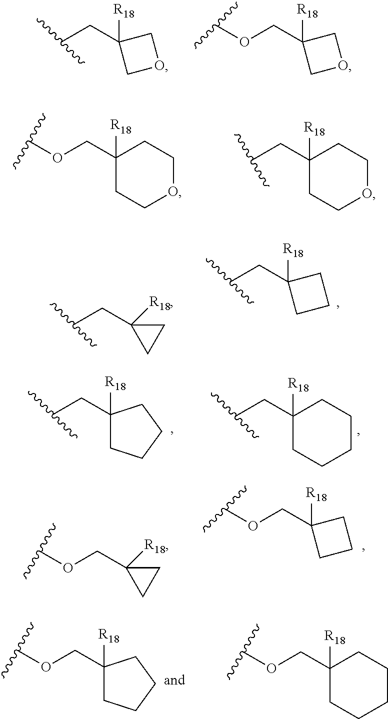

and wherein R₁₈ is selected from the group consisting of H, —OH, halogen, C₁-C₆ alkyl, —O—(C₁-C₆ alkyl) and C₁-C₆ haloalkyl is provided.

In an embodiment a compound of formula I wherein -A-B is selected from the group consisting

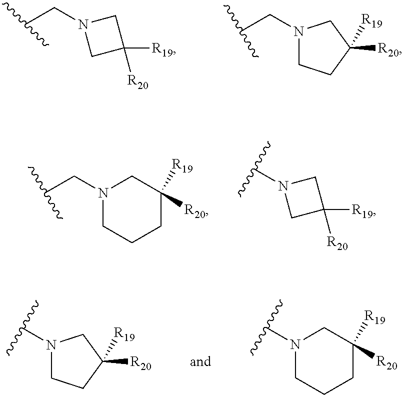

wherein R₁₉ and R₂₀ are each independently selected from the group consisting of H, —OH, halogen, C₁-C₆ alkyl, —O—(C₁-C₆ alkyl) and C₁-C₆ haloalkyl is provided.

In an embodiment a compound of formula I wherein -A-B is selected from the group consisting of

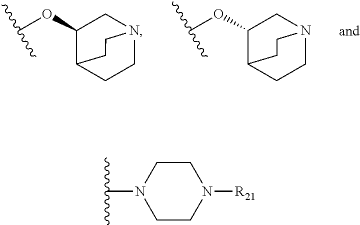

wherein R₂₁ is selected from the group consisting of H and C₁-C₆ alkyl is provided.

In an embodiment a compound of formula I wherein -A-B is selected from the group consisting of —OCH₃, —CN, —CH₂SO₂CH₃, —OCF₃, —CF₃, —CHF₂,

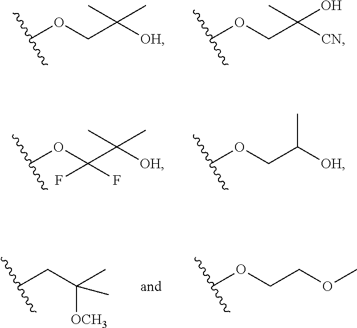

is provided.

In an embodiment a compound of formula II

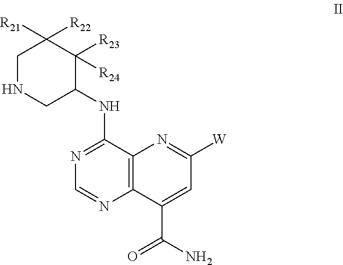

wherein:
R₂₁, R₂₂, R₂₃ and R₂₄ are each independently selected from the group consisting of H and halogen;
W is selected from the group consisting of optionally substituted 5-6 member heteroaromatic containing 1-4 heteroatoms, optionally substituted 5-6 member heterocyclic containing 1-4 heteroatoms, C₁-C₆ alkenyl (optionally substituted 5-6 member heterocyclic containing 1-4 heteroatoms), C₁-C₆ alkyl (optionally substituted 5-6 member heterocyclic containing 1-4 heteroatoms) and optionally substituted cyclohexene
or a pharmaceutically acceptable salt, prodrugs, enantiomer, mixture of enantiomers, diastereomers or mixture of diastereomers is provided.

In an embodiment a compound having the formula IIa is provided.

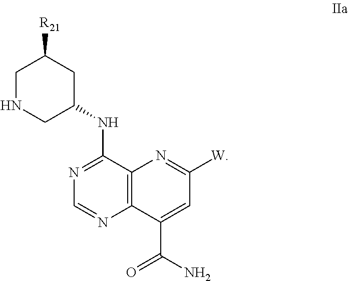

In an embodiment a compound having the formula IIb is provided.

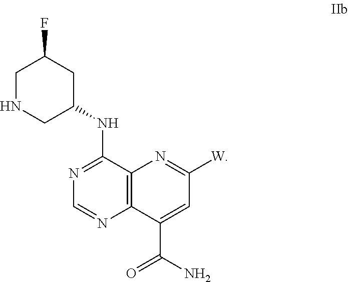

In an embodiment a compound having the formula IIc is provided.

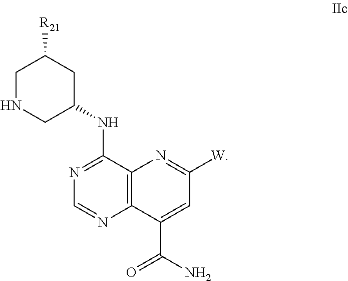

In an embodiment a compound having the formula IId is provided.

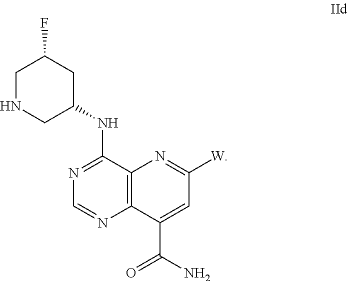

In another embodiment a compound having the formula IIe is provided.

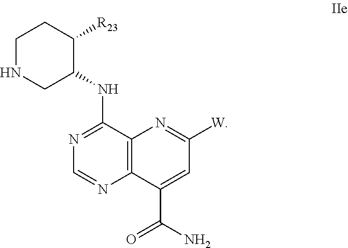

In another embodiment a compound having the formula IIf is provided.

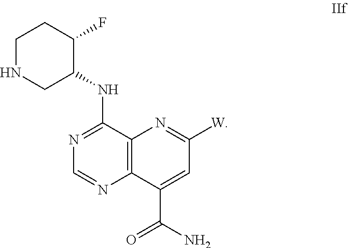

In another embodiment a compound having the formula IIg is provided.

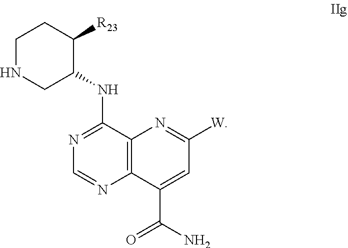

In another embodiment a compound having the formula IIh is provided.

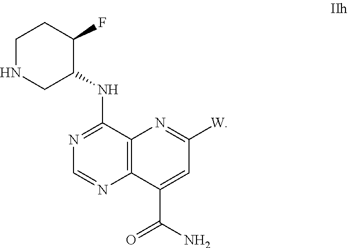

In another embodiment a compound having the formula IIl is provided.

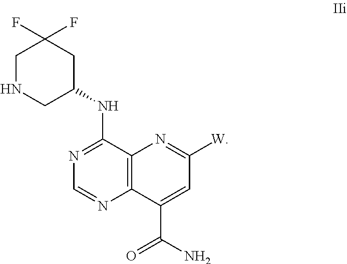

In another embodiment a compound having the formula IIj is provided.

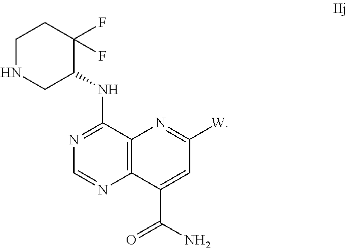

In another embodiment a compound having the formula IIk is provided.

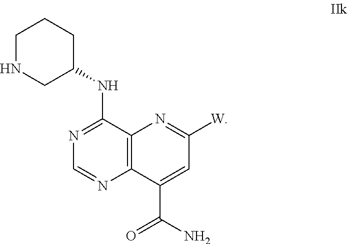

In another embodiment a compound wherein R₂₁, R₂₂, R₂₃ and R₂₄ are each H is provided.

In another embodiment a compound wherein R₂₁, R₂₃ and R₂₄ are H and R₂₂ is a halogen is provided.

In another embodiment a compound wherein R₂₂ is F is provided.

In another embodiment a compound wherein R₂₁, R₂₂, and R₂₃ are H and R₂₄ is halogen is provided.

In another embodiment a compound wherein R₂₄ is F is provided.

In another embodiment a compound wherein R₂₁ and R₂₂ are halogen and R₂₃ and R₂₄ are H is provided.

In another embodiment a compound wherein R₂₁ and R₂₂ are F is provided.

In another embodiment a compound wherein R₂₁ and R₂₂ are H and R₂₃ and R₂₄ are halogen.

In another embodiment a compound wherein R₂₃ and R₂₄ are F.

In another embodiment a compound wherein W is selected from the group consisting of

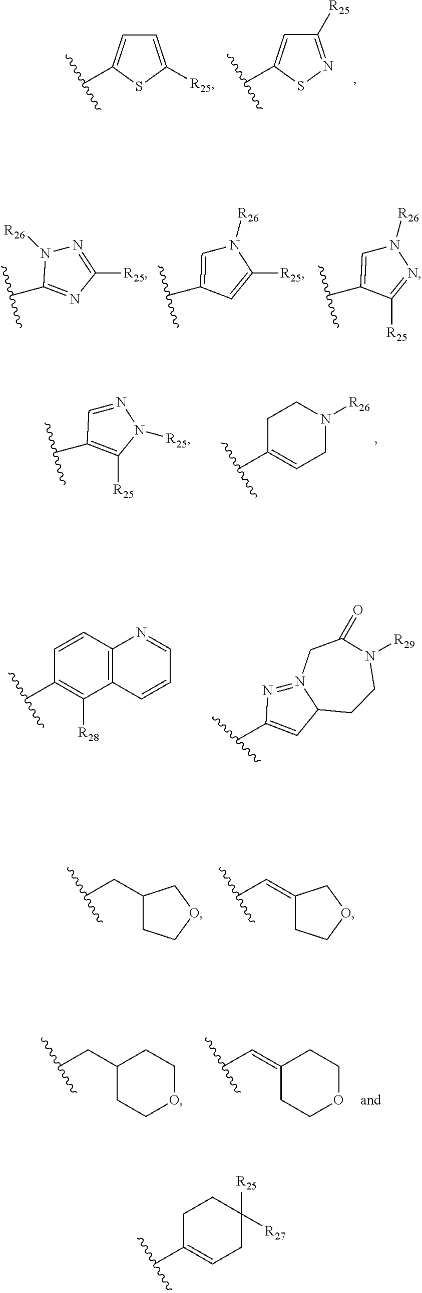

wherein R₂₅ and R₂₆ are independently selected from the group consisting of H, C₁-C₆ alkyl, C₃-C₆ cycloalkyl, C₁-C₆ haloalkyl, halogen, —CN, —CH₂—O—CH₃, —CH₂CH₂—O—CH₃, tetrahydropyranyl, morpholino,

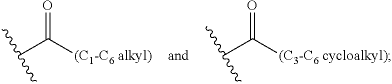

R₂₇ is selected from the group consisting of H, C₁-C₆ alkyl, C₃-C₆ cycloalkyl, C₁-C₆ haloalkyl

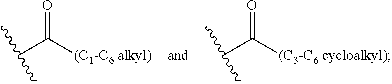

R₂₈ is selected from the group consisting of H, halogen, —O—C₁-C₆ alkyl, C₁-C₆ alkyl, C₁-C₆ haloalkyl and —CN; R₂₉ is selected from the group consisting of H and C₁-C₆ alkyl is provided.

In an embodiment, the compound of formula (I) is one of the following compounds:

| # | Structure |
|---|---|
| Example 1 | 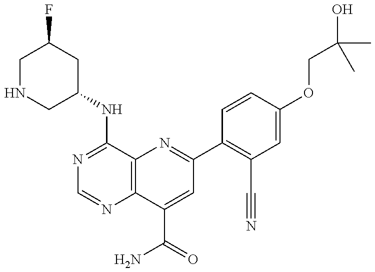 [SMILES: CC(C)(O)COc1ccc(-c2cc(C(N)=O)c3ncnc(N[C@@H]4CNC[C@@H](F)C4)c3n2)c(C#N)c1] |
| Example 10 | 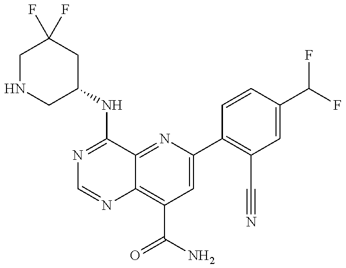 [SMILES: N#Cc1cc(C(F)F)ccc1-c1cc(C(N)=O)c2ncnc(N[C@@H]3CNCC(F)(F)C3)c2n1] |
| Example 11 | 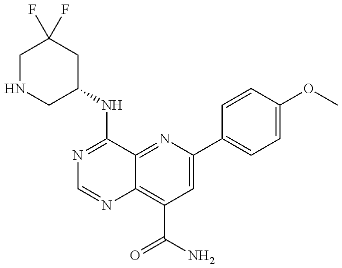 [SMILES: COc1ccc(-c2cc(C(N)=O)c3ncnc(N[C@@H]4CNCC(F)(F)C4)c3n2)cc1] |
| Example 12 | 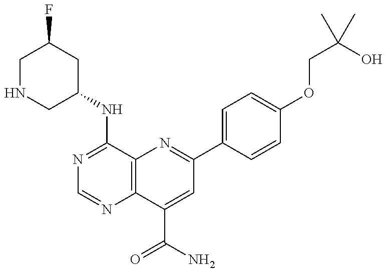 [SMILES: CC(C)(O)COc1ccc(-c2cc(C(N)=O)c3ncnc(N[C@@H]4CNC[C@@H](F)C4)c3n2)cc1] |
| Example 14 | 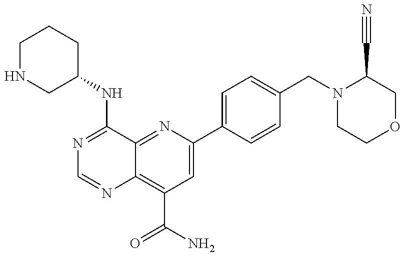 [SMILES: N#C[C@H]1COCCN1Cc1ccc(-c2cc(C(N)=O)c3ncnc(N[C@H]4CCCNC4)c3n2)cc1] |
| | Isomer 1 |
| | 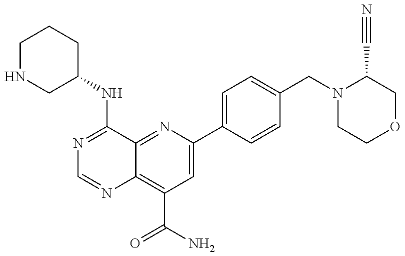 [SMILES: N#C[C@@H]1COCCN1Cc1ccc(-c2cc(C(N)=O)c3ncnc(N[C@H]4CCCNC4)c3n2)cc1] |
| | Isomer 2 |
| Example 15 | 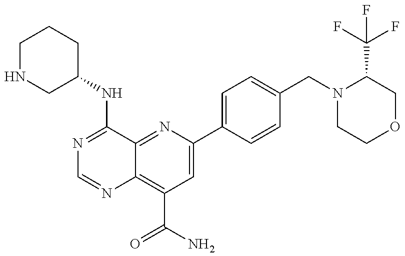 [SMILES: NC(=O)c1cc(-c2ccc(CN3CCOC[C@H]3C(F)(F)F)cc2)nc2c(N[C@H]3CCCNC3)ncnc12] |
| | Isomer 1 |
| | 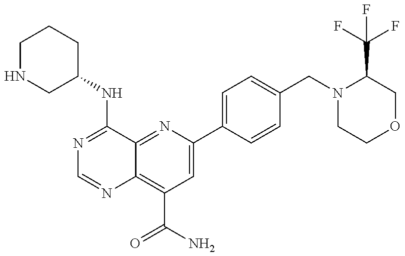 [SMILES: NC(=O)c1cc(-c2ccc(CN3CCOC[C@@H]3C(F)(F)F)cc2)nc2c(N[C@H]3CCCNC3)ncnc12] |
| | Isomer 2 |
| Example 16 | 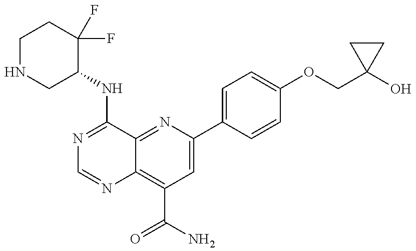 [SMILES: NC(=O)c1cc(-c2ccc(OCC3(O)CC3)cc2)nc2c(N[C@@H]3CNCCC3(F)F)ncnc12] |
| Example 22 | 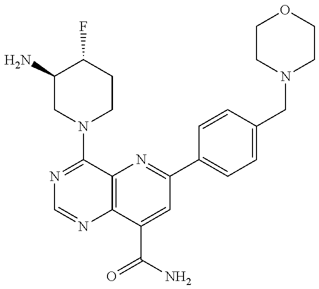 [SMILES: NC(=O)c1cc(-c2ccc(CN3CCOCC3)cc2)nc2c(N3CC[C@@H](F)[C@H](N)C3)ncnc12] |
| | Isomer 1 |
| | 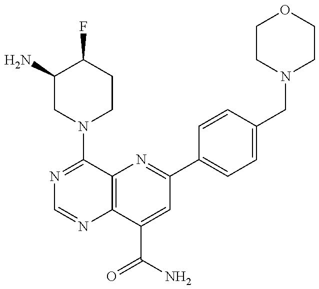 [SMILES: NC(=O)c1cc(-c2ccc(CN3CCOCC3)cc2)nc2c(N3CC[C@H](F)[C@H](N)C3)ncnc12] |
| | Isomer 2 |
| | 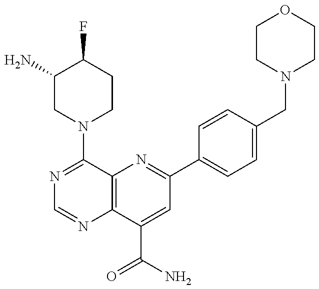 [SMILES: NC(=O)c1cc(-c2ccc(CN3CCOCC3)cc2)nc2c(N3CC[C@H](F)[C@@H](N)C3)ncnc12] |
| | Isomer 3 |
| | 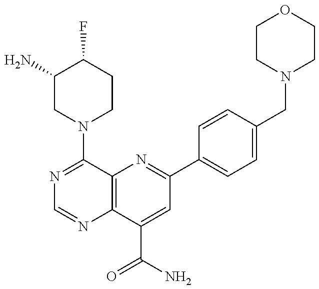 [SMILES: NC(=O)c1cc(-c2ccc(CN3CCOCC3)cc2)nc2c(N3CC[C@@H](F)[C@@H](N)C3)ncnc12] |
| | Isomer 4 |
| Example 24 | 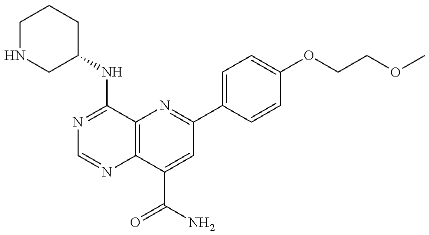 [SMILES: COCCOc1ccc(-c2cc(C(N)=O)c3ncnc(N[C@H]4CCCNC4)c3n2)cc1] |
| Example 26 | 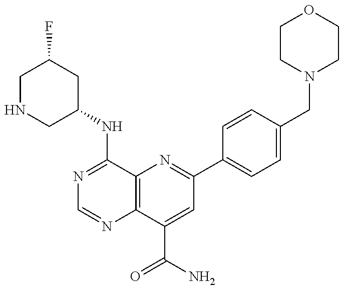 [SMILES: NC(=O)c1cc(-c2ccc(CN3CCOCC3)cc2)nc2c(N[C@@H]3CNC[C@H](F)C3)ncnc12] |
| | Isomer 1 |
| | 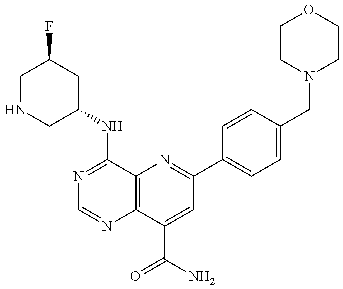 [SMILES: NC(=O)c1cc(-c2ccc(CN3CCOCC3)cc2)nc2c(N[C@@H]3CNC[C@@H](F)C3)ncnc12] |
| | Isomer 2 |
| | 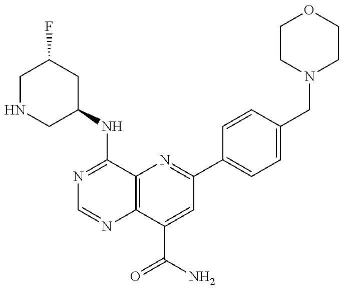 [SMILES: NC(=O)c1cc(-c2ccc(CN3CCOCC3)cc2)nc2c(N[C@H]3CNC[C@H](F)C3)ncnc12] |
| | Isomer 3 |
| | 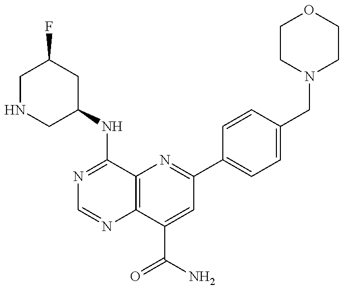 [SMILES: NC(=O)c1cc(-c2ccc(CN3CCOCC3)cc2)nc2c(N[C@H]3CNC[C@@H](F)C3)ncnc12] |
| | Isomer 4 |
| Example 27 | 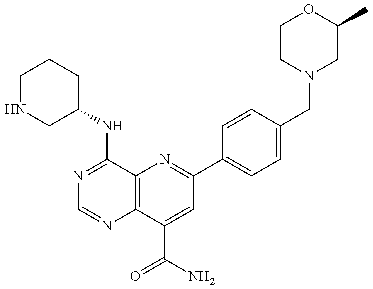 [SMILES: C[C@H]1CN(Cc2ccc(-c3cc(C(N)=O)c4ncnc(N[C@H]5CCCNC5)c4n3)cc2)CCO1] |
| Example 28 | 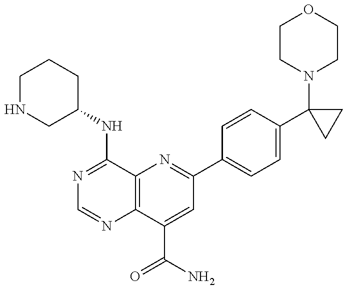 [SMILES: NC(=O)c1cc(-c2ccc(C3(N4CCOCC4)CC3)cc2)nc2c(N[C@H]3CCCNC3)ncnc12] |
| Example 29 | 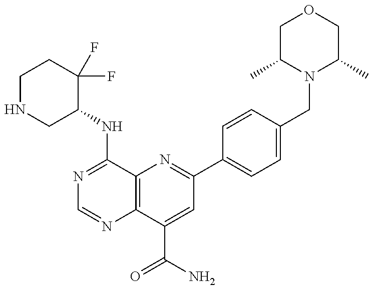 [SMILES: C[C@@H]1COC[C@H](C)N1Cc1ccc(-c2cc(C(N)=O)c3ncnc(N[C@@H]4CNCCC4(F)F)c3n2)cc1] |
| Example 30 | 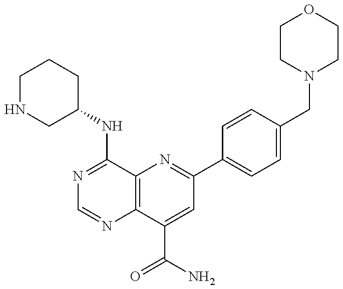 [SMILES: NC(=O)c1cc(-c2ccc(CN3CCOCC3)cc2)nc2c(N[C@H]3CCCNC3)ncnc12] |
| Example 31 | 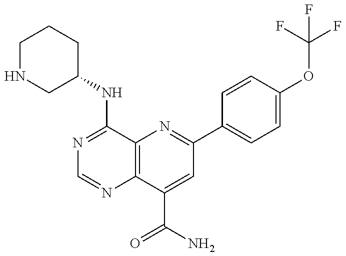 [SMILES: NC(=O)c1cc(-c2ccc(OC(F)(F)F)cc2)nc2c(N[C@H]3CCCNC3)ncnc12] |
| Example 32 | 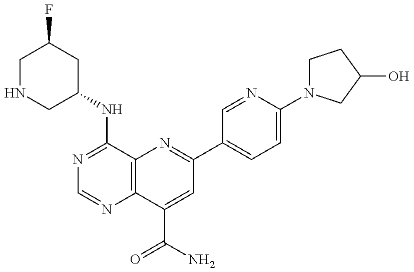 [SMILES: NC(=O)c1cc(-c2ccc(N3CCC(O)C3)nc2)nc2c(N[C@@H]3CNC[C@@H](F)C3)ncnc12] |
| Example 33 | 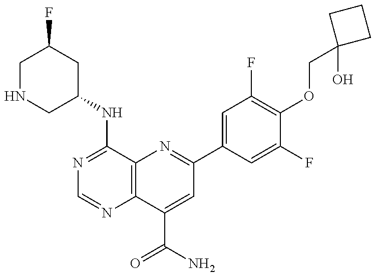 [SMILES: NC(=O)c1cc(-c2cc(F)c(OCC3(O)CCC3)c(F)c2)nc2c(N[C@@H]3CNC[C@@H](F)C3)ncnc12] |
| Example 34 | 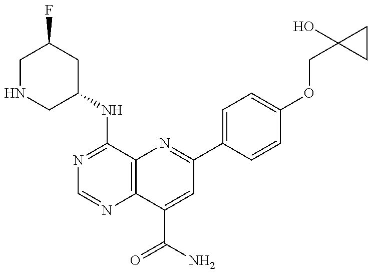 [SMILES: NC(=O)c1cc(-c2ccc(OCC3(O)CC3)cc2)nc2c(N[C@@H]3CNC[C@@H](F)C3)ncnc12] |
| Example 35 | 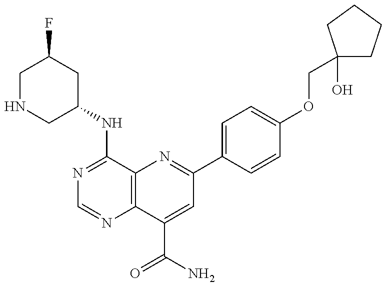 [SMILES: NC(=O)c1cc(-c2ccc(OCC3(O)CCCC3)cc2)nc2c(N[C@@H]3CNC[C@@H](F)C3)ncnc12] |
| Example 37 | 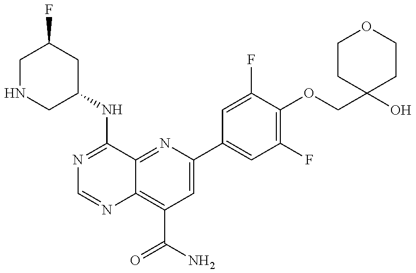 [SMILES: NC(=O)c1cc(-c2cc(F)c(OCC3(O)CCOCC3)c(F)c2)nc2c(N[C@@H]3CNC[C@@H](F)C3)ncnc12] |
| Example 38 | 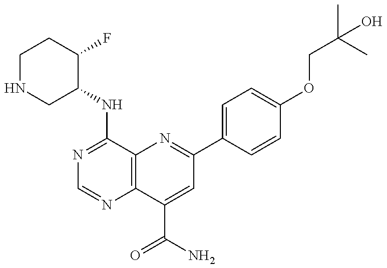 [SMILES: CC(C)(O)COc1ccc(-c2cc(C(N)=O)c3ncnc(N[C@@H]4CNCC[C@@H]4F)c3n2)cc1] |
| Example 39 | 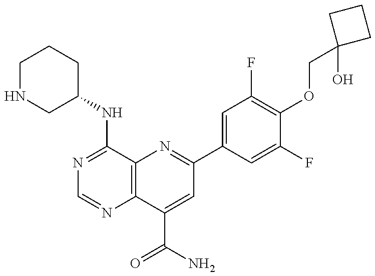 [SMILES: NC(=O)c1cc(-c2cc(F)c(OCC3(O)CCC3)c(F)c2)nc2c(N[C@H]3CCCNC3)ncnc12] |
| Example 40 | 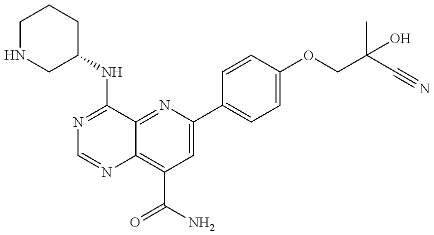 [SMILES: CC(O)(C#N)COc1ccc(-c2cc(C(N)=O)c3ncnc(N[C@H]4CCCNC4)c3n2)cc1] |
| Example 41 | 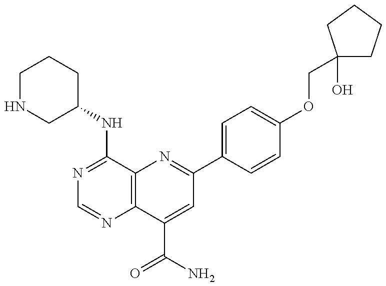 [SMILES: NC(=O)c1cc(-c2ccc(OCC3(O)CCCC3)cc2)nc2c(N[C@H]3CCCNC3)ncnc12] |
| Example 42 | 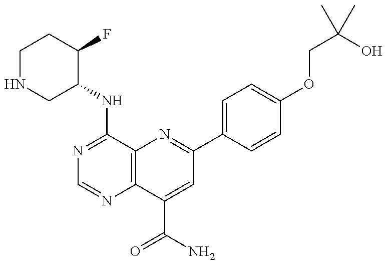 [SMILES: CC(C)(O)COc1ccc(-c2cc(C(N)=O)c3ncnc(N[C@@H]4CNCC[C@H]4F)c3n2)cc1] |
| Example 43 | 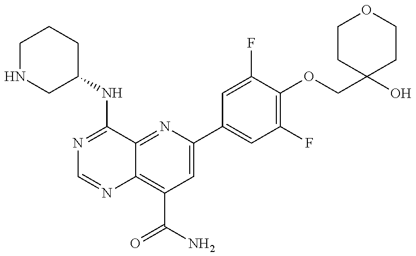 [SMILES: NC(=O)c1cc(-c2cc(F)c(OCC3(O)CCOCC3)c(F)c2)nc2c(N[C@H]3CCCNC3)ncnc12] |
| Example 44 | 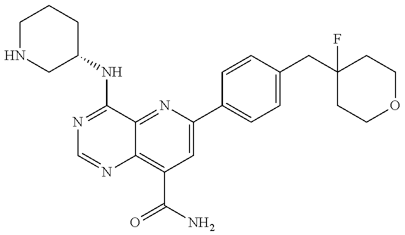 [SMILES: NC(=O)c1cc(-c2ccc(CC3(F)CCOCC3)cc2)nc2c(N[C@H]3CCCNC3)ncnc12] |
| Example 45 | 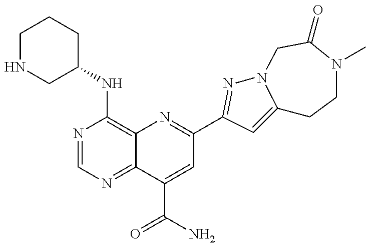 [SMILES: CN1CCc2cc(-c3cc(C(N)=O)c4ncnc(N[C@H]5CCCNC5)c4n3)nn2CC1=O] |
| Example 46 | 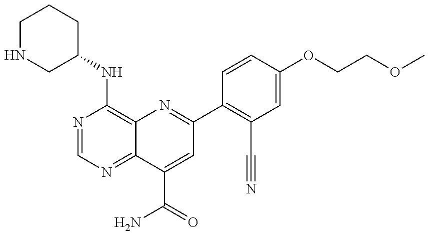 [SMILES: COCCOc1ccc(-c2cc(C(N)=O)c3ncnc(N[C@H]4CCCNC4)c3n2)c(C#N)c1] |
| Example 47 | 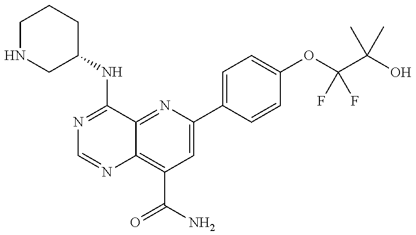 [SMILES: CC(C)(O)C(F)(F)Oc1ccc(-c2cc(C(N)=O)c3ncnc(N[C@H]4CCCNC4)c3n2)cc1] |
| Example 48 | 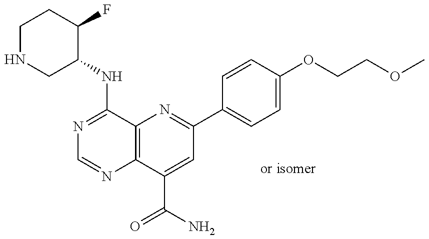 [SMILES: COCCOc1ccc(-c2cc(C(N)=O)c3ncnc(N[C@@H]4CNCC[C@H]4F)c3n2)cc1] |
| Example 49 | 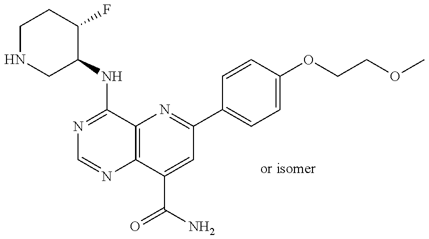 [SMILES: COCCOc1ccc(-c2cc(C(N)=O)c3ncnc(N[C@H]4CNCC[C@@H]4F)c3n2)cc1] |
| Example 50 | 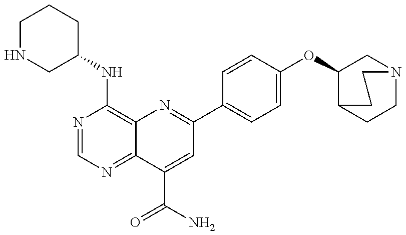 [SMILES: NC(=O)c1cc(-c2ccc(O[C@H]3CN4CCC3CC4)cc2)nc2c(N[C@H]3CCCNC3)ncnc12] |
| Example 51 | 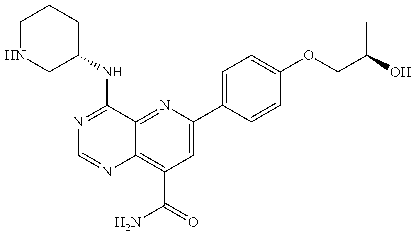 [SMILES: C[C@@H](O)COc1ccc(-c2cc(C(N)=O)c3ncnc(N[C@H]4CCCNC4)c3n2)cc1] |
| Example 52 | 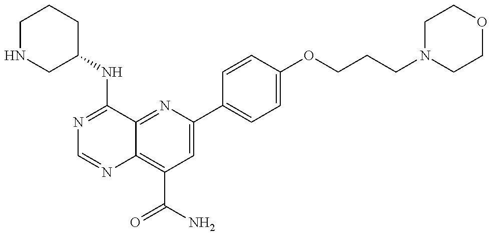 [SMILES: NC(=O)c1cc(-c2ccc(OCCCN3CCOCC3)cc2)nc2c(N[C@H]3CCCNC3)ncnc12] |
| Example 53 | 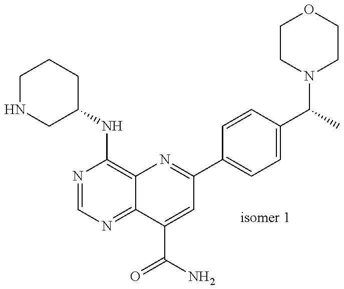 [SMILES: C[C@H](c1ccc(-c2cc(C(N)=O)c3ncnc(N[C@H]4CCCNC4)c3n2)cc1)N1CCOCC1] |
| | 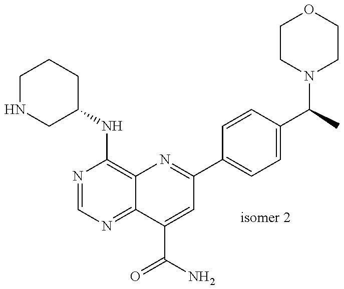 [SMILES: C[C@@H](c1ccc(-c2cc(C(N)=O)c3ncnc(N[C@H]4CCCNC4)c3n2)cc1)N1CCOCC1] |
| Example 54 | 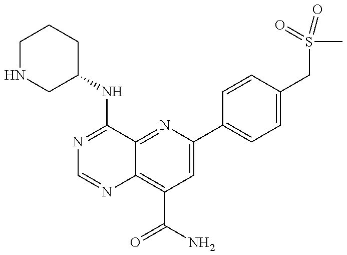 [SMILES: CS(=O)(=O)Cc1ccc(-c2cc(C(N)=O)c3ncnc(N[C@H]4CCCNC4)c3n2)cc1] |
| Example 55 | 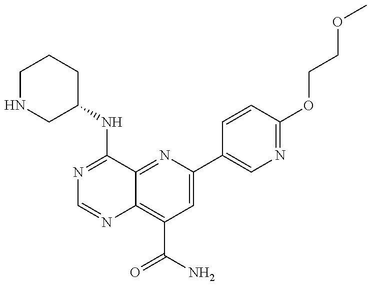 [SMILES: COCCOc1ccc(-c2cc(C(N)=O)c3ncnc(N[C@H]4CCCNC4)c3n2)cn1] |
| Example 56 | 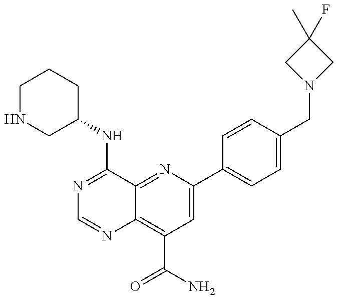 [SMILES: CC1(F)CN(Cc2ccc(-c3cc(C(N)=O)c4ncnc(N[C@H]5CCCNC5)c4n3)cc2)C1] |
| Example 57 | 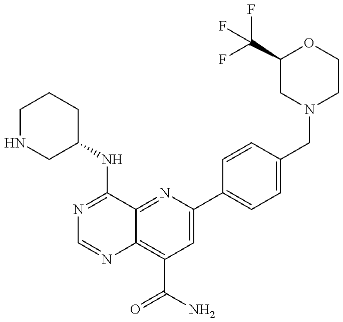 [SMILES: NC(=O)c1cc(-c2ccc(CN3CCO[C@H](C(F)(F)F)C3)cc2)nc2c(N[C@H]3CCCNC3)ncnc12] |
| Example 58 | 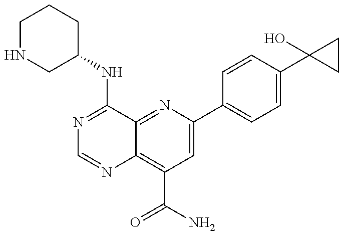 [SMILES: NC(=O)c1cc(-c2ccc(C3(O)CC3)cc2)nc2c(N[C@H]3CCCNC3)ncnc12] |
| Example 59 | 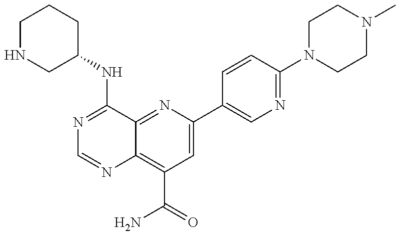 [SMILES: CN1CCN(c2ccc(-c3cc(C(N)=O)c4ncnc(N[C@H]5CCCNC5)c4n3)cn2)CC1] |
| Example 60 | 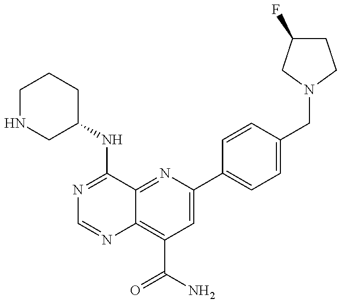 [SMILES: NC(=O)c1cc(-c2ccc(CN3CC[C@H](F)C3)cc2)nc2c(N[C@H]3CCCNC3)ncnc12] |
| Example 61 | 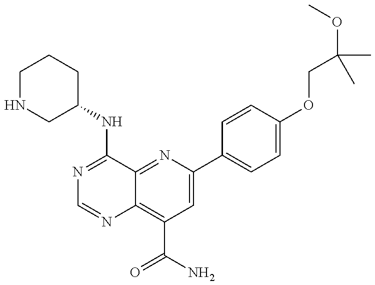 [SMILES: COC(C)(C)COc1ccc(-c2cc(C(N)=O)c3ncnc(N[C@H]4CCCNC4)c3n2)cc1] |
| Example 62 | 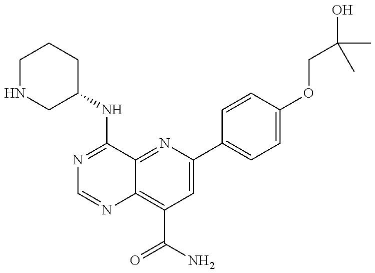 [SMILES: CC(C)(O)COc1ccc(-c2cc(C(N)=O)c3ncnc(N[C@H]4CCCNC4)c3n2)cc1] |
| Example 63 | 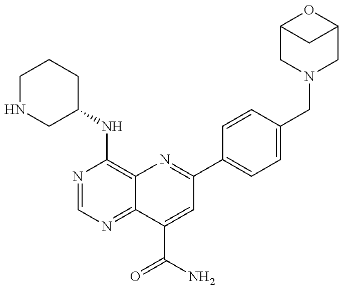 [SMILES: NC(=O)c1cc(-c2ccc(CN3CC4CC(C3)O4)cc2)nc2c(N[C@H]3CCCNC3)ncnc12] |
| Example 64 | 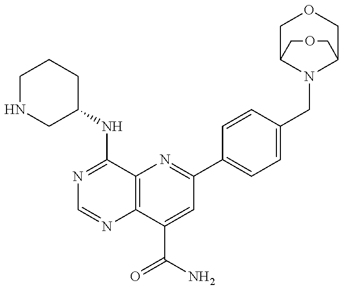 [SMILES: NC(=O)c1cc(-c2ccc(CN3C4COCC3COC4)cc2)nc2c(N[C@H]3CCCNC3)ncnc12] |
| Example 65 | 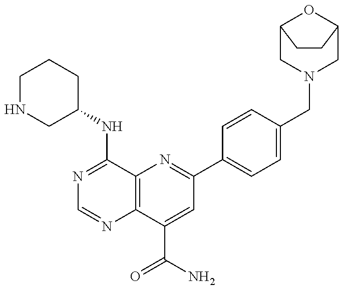 [SMILES: NC(=O)c1cc(-c2ccc(CN3CC4CCC(C3)O4)cc2)nc2c(N[C@H]3CCCNC3)ncnc12] |
| Example 66 | 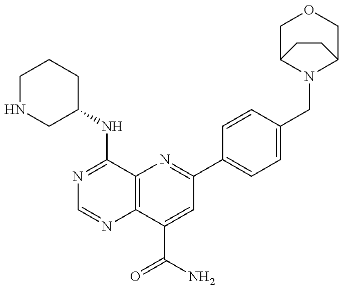 [SMILES: NC(=O)c1cc(-c2ccc(CN3C4CCC3COC4)cc2)nc2c(N[C@H]3CCCNC3)ncnc12] |
| Example 67 | 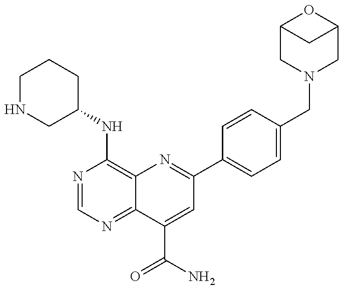 [SMILES: NC(=O)c1cc(-c2ccc(CN3CC4CC(C3)O4)cc2)nc2c(N[C@H]3CCCNC3)ncnc12] |
| Example 68 | 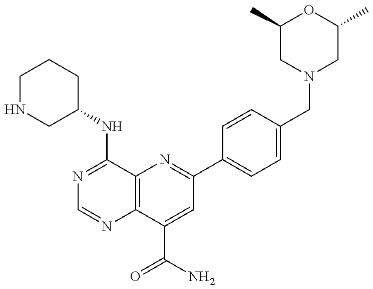 [SMILES: C[C@@H]1CN(Cc2ccc(-c3cc(C(N)=O)c4ncnc(N[C@H]5CCCNC5)c4n3)cc2)C[C@@H](C)O1] |
| Example 69 | 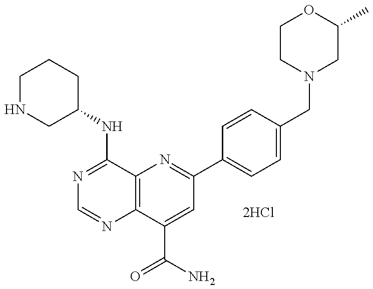 [SMILES: C[C@@H]1CN(Cc2ccc(-c3cc(C(N)=O)c4ncnc(N[C@H]5CCCNC5)c4n3)cc2)CCO1] |
| Example 70 | 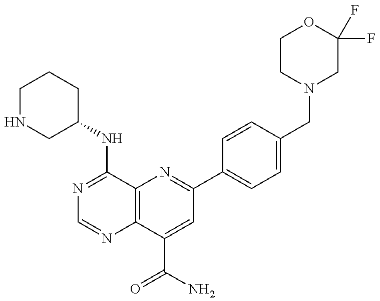 [SMILES: NC(=O)c1cc(-c2ccc(CN3CCOC(F)(F)C3)cc2)nc2c(N[C@H]3CCCNC3)ncnc12] |
| Example 71 | 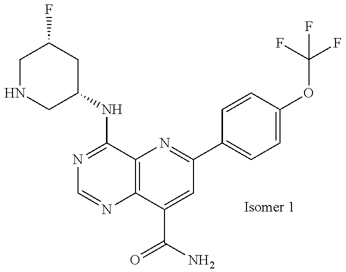 [SMILES: NC(=O)c1cc(-c2ccc(OC(F)(F)F)cc2)nc2c(N[C@@H]3CNC[C@H](F)C3)ncnc12] |
| | cis assumed; |
| | abs assumed |
| | 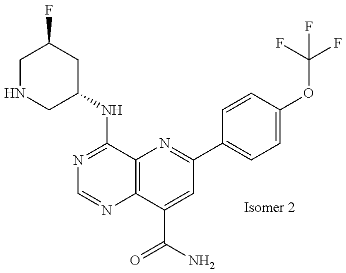 [SMILES: NC(=O)c1cc(-c2ccc(OC(F)(F)F)cc2)nc2c(N[C@@H]3CNC[C@@H](F)C3)ncnc12] |
| | trans assumed; |
| | abs assumed |
| | 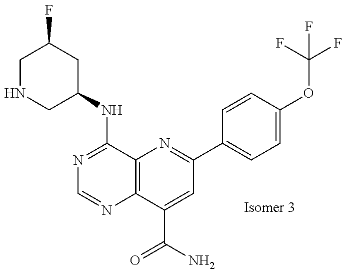 [SMILES: NC(=O)c1cc(-c2ccc(OC(F)(F)F)cc2)nc2c(N[C@H]3CNC[C@@H](F)C3)ncnc12] |
| | cis assumed; |
| | abs assumed |
| | 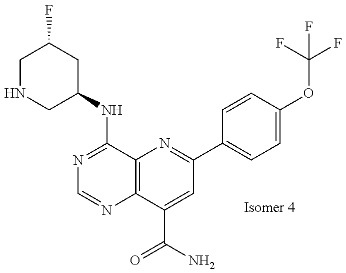 [SMILES: NC(=O)c1cc(-c2ccc(OC(F)(F)F)cc2)nc2c(N[C@H]3CNC[C@H](F)C3)ncnc12] |
| | trans assumed; |
| | abs assumed |
| Example 72 | 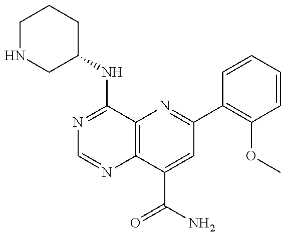 [SMILES: COc1ccccc1-c1cc(C(N)=O)c2ncnc(N[C@H]3CCCNC3)c2n1] |
| Example 76 | 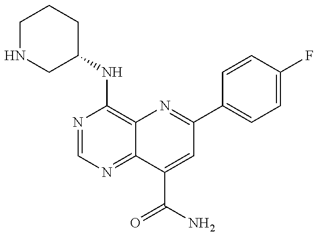 [SMILES: NC(=O)c1cc(-c2ccc(F)cc2)nc2c(N[C@H]3CCCNC3)ncnc12] |
| Example 82 | 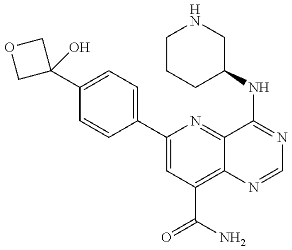 [SMILES: NC(=O)c1cc(-c2ccc(C3(O)COC3)cc2)nc2c(N[C@H]3CCCNC3)ncnc12] |
| Example 86 | 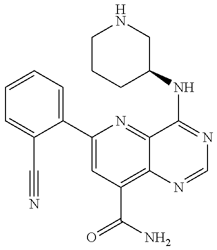 [SMILES: N#Cc1ccccc1-c1cc(C(N)=O)c2ncnc(N[C@H]3CCCNC3)c2n1] |
| Example 87 | 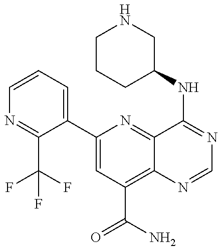 [SMILES: NC(=O)c1cc(-c2cccnc2C(F)(F)F)nc2c(N[C@H]3CCCNC3)ncnc12] |

or a pharmaceutically acceptable salt, prodrugs, enantiomer, mixture of enantiomers, diastereomers or mixture of diastereomers thereof.

In an embodiment, the compound of formula (II) is one of the following compounds:

| | |
|---|---|
| Example 2 | 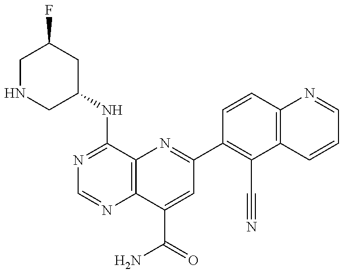 [SMILES: N#Cc1c(-c2cc(C(N)=O)c3ncnc(N[C@@H]4CNC[C@@H](F)C4)c3n2)ccc2ncccc12] |
| Example 3 | 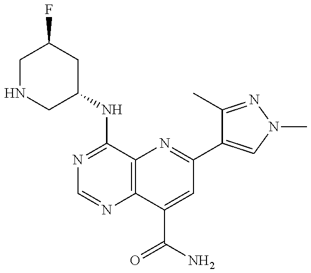 [SMILES: Cc1nn(C)cc1-c1cc(C(N)=O)c2ncnc(N[C@@H]3CNC[C@@H](F)C3)c2n1] |
| Example 4 | 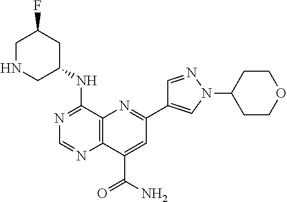 [SMILES: NC(=O)c1cc(-c2cnn(C3CCOCC3)c2)nc2c(N[C@@H]3CNC[C@@H](F)C3)ncnc12] |
| Example 5 | 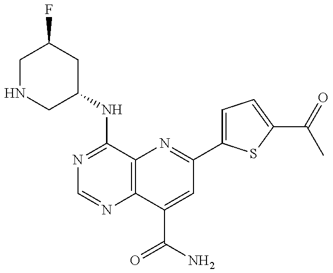 [SMILES: CC(=O)c1ccc(-c2cc(C(N)=O)c3ncnc(N[C@@H]4CNC[C@@H](F)C4)c3n2)s1] |
| Example 6 | 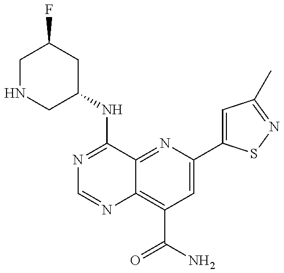 [SMILES: Cc1cc(-c2cc(C(N)=O)c3ncnc(N[C@@H]4CNC[C@@H](F)C4)c3n2)sn1] |
| Example 7 | 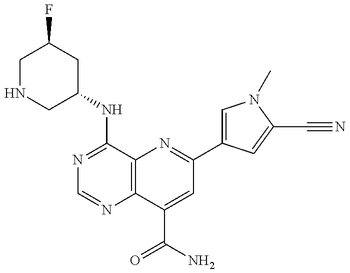 [SMILES: Cn1cc(-c2cc(C(N)=O)c3ncnc(N[C@@H]4CNC[C@@H](F)C4)c3n2)cc1C#N] |
| Example 8 | 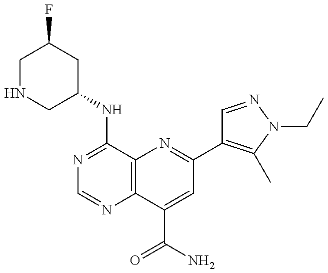 [SMILES: CCn1ncc(-c2cc(C(N)=O)c3ncnc(N[C@@H]4CNC[C@@H](F)C4)c3n2)c1C] |
| Example 9 | 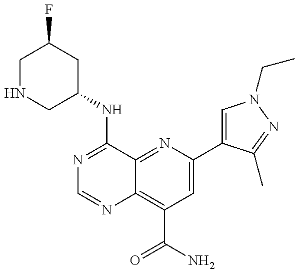 [SMILES: CCn1cc(-c2cc(C(N)=O)c3ncnc(N[C@@H]4CNC[C@@H](F)C4)c3n2)c(C)n1] |
| Example 13 | 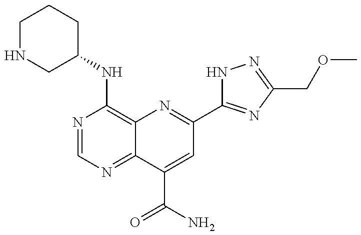 [SMILES: COCc1n[nH]c(-c2cc(C(N)=O)c3ncnc(N[C@H]4CCCNC4)c3n2)n1] |
| Example 17 | 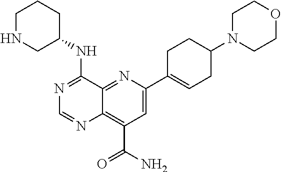 [SMILES: NC(=O)c1cc(C2=CCC(N3CCOCC3)CC2)nc2c(N[C@H]3CCCNC3)ncnc12] |
| Example 19 | 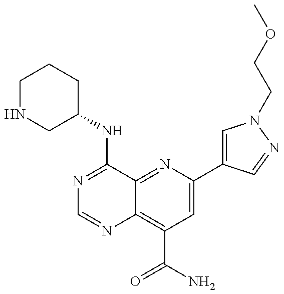 [SMILES: COCCn1cc(-c2cc(C(N)=O)c3ncnc(N[C@H]4CCCNC4)c3n2)cn1] |
| Example 36 | 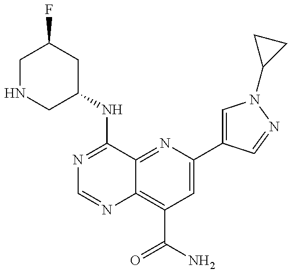 [SMILES: NC(=O)c1cc(-c2cnn(C3CC3)c2)nc2c(N[C@@H]3CNC[C@@H](F)C3)ncnc12] |
| Example 73 | 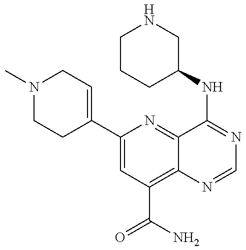 [SMILES: CN1CC=C(c2cc(C(N)=O)c3ncnc(N[C@H]4CCCNC4)c3n2)CC1] |
| Example 74 | 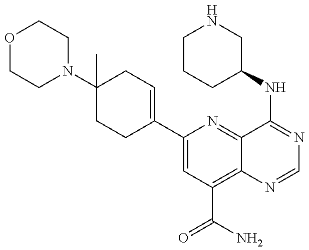 [SMILES: CC1(N2CCOCC2)CC=C(c2cc(C(N)=O)c3ncnc(N[C@H]4CCCNC4)c3n2)CC1] |
| Example 75 | 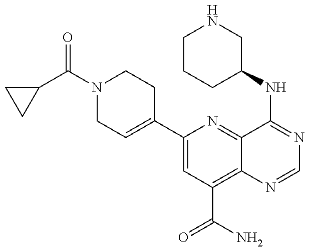 [SMILES: NC(=O)c1cc(C2=CCN(C(=O)C3CC3)CC2)nc2c(N[C@H]3CCCNC3)ncnc12] |
| Example 77 | 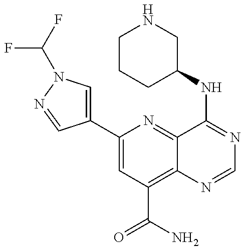 [SMILES: NC(=O)c1cc(-c2cnn(C(F)F)c2)nc2c(N[C@H]3CCCNC3)ncnc12] |
| Example 78 | 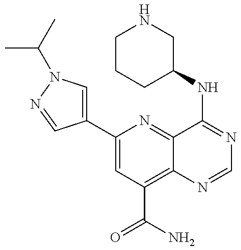 [SMILES: CC(C)n1cc(-c2cc(C(N)=O)c3ncnc(N[C@H]4CCCNC4)c3n2)cn1] |
| Example 79 | 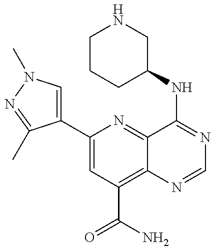 [SMILES: Cc1nn(C)cc1-c1cc(C(N)=O)c2ncnc(N[C@H]3CCCNC3)c2n1] |
| Example 80 | 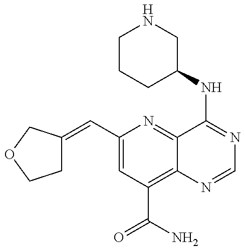 [SMILES: NC(=O)c1cc(/C=C2\CCOC2)nc2c(N[C@H]3CCCNC3)ncnc12] |
| Example 81 | 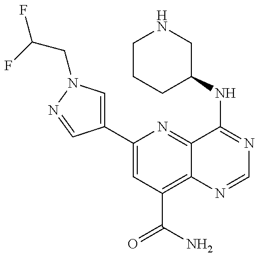 [SMILES: NC(=O)c1cc(-c2cnn(CC(F)F)c2)nc2c(N[C@H]3CCCNC3)ncnc12] |
| Example 83 | 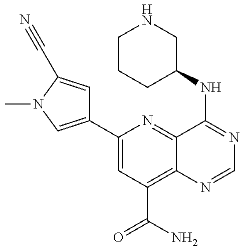 [SMILES: Cn1cc(-c2cc(C(N)=O)c3ncnc(N[C@H]4CCCNC4)c3n2)cc1C#N] |
| Example 84 | 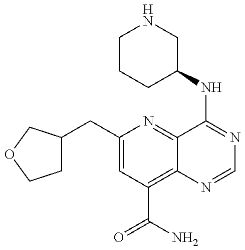 [SMILES: NC(=O)c1cc(CC2CCOC2)nc2c(N[C@H]3CCCNC3)ncnc12] |
| Example 85 | 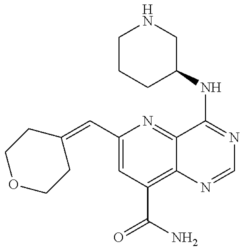 [SMILES: NC(=O)c1cc(C=C2CCOCC2)nc2c(N[C@H]3CCCNC3)ncnc12] |
| Example 88 | 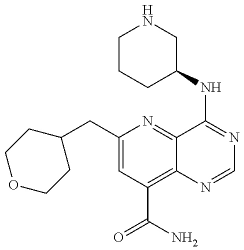 [SMILES: NC(=O)c1cc(CC2CCOCC2)nc2c(N[C@H]3CCCNC3)ncnc12] |

and

| | |
|---|---|
| Example 45 | 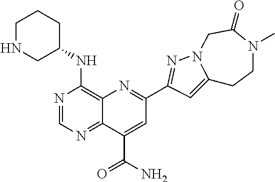 [SMILES: CN1CCc2cc(-c3cc(C(N)=O)c4ncnc(N[C@H]5CCCNC5)c4n3)nn2CC1=O] |

or a pharmaceutically acceptable salt, pro rugs, enantiomer, mixture of enantiomers, diastereomers or mixture of diastereomers thereof.

In an embodiment, a pharmaceutical composition contains a compound of formula (I) or (II) as defined above and a pharmaceutically acceptable adjuvant, carrier, or vehicle.

In an embodiment, a method is provided in which a therapeutically effective amount of the compound having the structure of formula (I) or (II) or a physiologically acceptable salt thereof as defined above is administered to a patient having an HPK1-mediated disorder. The HPK-1 mediated disorder may be a cancer. The cancer may be one or more of cancer of the breast, bladder, bone, brain, central and peripheral nervous system, colon, endocrine glands, esophagus, endometrium, germ cells, head and neck, kidney, liver, lung, larynx and hypopharynx, ovary, pancreas, prostate, rectum, renal, small intestine, soft tissue, testis, stomach, skin, ureter, vagina, and vulva. A therapeutically effective amount of the compound may be one of the following ranges: a range consisting of 0.1 to 100 mg/kg of body weight of the patient, 0.1 to 50 mg/kg of body weight of the patient, 0.5 to 50 mg/kg of body weight of the patient, 1 to 20 mg/kg of body weight of the patient, 5 to 20 mg/kg, 10 to 20 mg/kg, 10-50 mg/kg of body weight of the patient, or 10-100 mg/kg of body weight of the patient or any other dosing range disclosed herein. The compound may be administered to the patient continuously, multiple times daily, once daily, once every other day, weekly, bi-weekly, monthly, or bi-monthly. The compound may be administered orally, parenterally, by inhalation spray, topically, rectally, nasally, buccally, vaginally, or via an implanted reservoir. The compound may be administered subcutaneously, intravenously, intramuscularly, intra-articularly, intra-synovially, intrasternally, intrathecally, intrahepaticly, intralesionally, and by intracranial injection or infusion technique.

In an embodiment, a method is provided in which a therapeutically effective amount of the pharmaceutical composition or a physiologically acceptable salt thereof is administered to a patient having an HPK1-mediated disorder. The HPK1-mediated disorder may be a cancer. The cancer may be one or more of breast, bladder, bone, brain, central and peripheral nervous system, colon, endocrine glands, esophagus, endometrium, germ cells, head and neck, kidney, liver, lung, larynx and hypopharynx, ovary, pancreas, prostate, rectum, renal, small intestine, soft tissue, testis, stomach, skin, ureter, vagina, and vulva. A therapeutically effective amount of the pharmaceutical composition may be a range 0.1 to 100 mg/kg of body weight of the patient, 0.1 to 50 mg/kg of body weight of the patient, 0.5 to 50 mg/kg of body weight of the patient, 1 to 20 mg/kg of body weight of the patient, 5 to 20 mg/kg, 10 to 20 mg/kg of body weight of the patient, or any other dosing range disclosed herein. The pharmaceutical composition may be administered to the patient continuously, multiple times daily, once daily, once every other day, weekly, bi-weekly, monthly, or bi-monthly. The pharmaceutical composition may be administered orally, parenterally, by inhalation spray, topically, rectally, nasally, buccally, vaginally, or via an implanted reservoir. The pharmaceutical composition may be administered subcutaneously, intravenously, intramuscularly, intra-articularly, intra-synovially, intrasternally, intrathecally, intrahepaticly, intralesionally, and by intracranial injection or infusion technique.

In an embodiment, a kit is provided that includes a therapeutically effective amount of the compound of formula (I) or (II) as defined above or a physiologically acceptable salt or prodrug thereof; and instructions for use of the compound.

Additional features, advantages, and embodiments of the disclosed subject matter may be set forth or apparent from consideration of the following detailed description and claims. Moreover, it is to be understood that both the foregoing summary and the following detailed description are exemplary and are intended to provide further explanation without limiting the scope of the claims.

### DETAILED DESCRIPTION

Provided herein is a chemical structure of the general formula (I) and (II) or a prodrug or pharmaceutically acceptable salt thereof, including mixtures thereof in all ratios, preferably for use in the treatment and/or prevention of cancer and inflammatory and/or autoimmune diseases or symptoms thereof. The compounds disclosed herein can inhibit HPK1 activity. The compounds disclosed herein have the general formula (I) and (II):

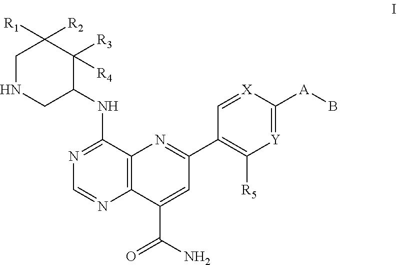

wherein:
R₁, R₂, R₃ and R₄ are each independently selected from the group consisting of H and halogen;
R₅ is selected from the group consisting of H, halogen, —O—C₁-C₆ alkyl, C₁-C₆ alkyl, C₁-C₆ haloalkyl and —CN;
X is selected from the group consisting of N and CR₆
Y is selected from the group consisting of N and CR₇
R₆ and R₇ are each independently selected from the group consisting of H, halogen and C₁-C₆ alkyl;
A is absent or selected from the group consisting of (—CH₂—)ₙ, —O—(—CH₂—)ₒ—, —O— and

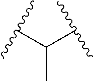 [SMILES: *C(*)C]

n is selected from the group consisting of 1, 2, 3 and 4;
o is selected from the group consisting of 1, 2, 3 and 4;
B is absent or selected from the group consisting of H, CN, halogen, optionally substituted C₆-C₁₄ aryl, optionally substituted C₂-C₁₄ heteroaryl, optionally substituted C₁-C₁₄ heterocyclic, optionally substituted C₁-C₈alkyl, C₁-C₈ haloalkyl, optionally substituted C₁-C₈ alkyl interrupted by 1-4 heteroatoms, and optionally substituted C₃-C₁₄ cycloalkyl;
or a pharmaceutically acceptable salt, prodrugs, enantiomer, mixture of enantiomers, diastereomers or mixture of diastereomers and

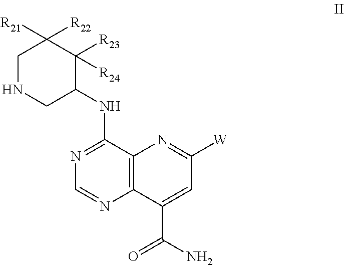

wherein:
R₂₁, R₂₂, R₂₃ and R₂₄ are each independently selected from the group consisting of H and halogen;
W is selected from the group consisting of optionally substituted 5-6 member heteroaromatic containing 1-4 heteroatoms, optionally substituted 5-6 member heterocyclic containing 1-4 heteroatoms, C₁-C₆ alkenyl (optionally substituted 5-6 member heterocyclic containing 1-4 heteroatoms), C₁-C₆ alkyl (optionally substituted 5-6 member heterocyclic containing 1-4 heteroatoms) and optionally substituted cyclohexene
or a pharmaceutically acceptable salt, prodrugs, enantiomer, mixture of enantiomers, diastereomers or mixture of diastereomers.

An alkyl can be a straight or branched alkyl group. Exemplary alkyl groups include, without limitation, methyl, ethyl, propyl, isopropyl, butyl, isobutyl, and tert-butyl. The number of carbon atoms in the alkyl is not particularly limited.

A halogen may refer to F, Cl, Br, or I.

A heterocyclyl is a univalent group formed by removing a hydrogen atom from any ring atom of a heterocyclic compound. It may include one or more heteroatoms. A heteroatom may refer to one or more of oxygen, sulfur, nitrogen, phosphorous, or any oxidized form thereof. A heterocycle, heterocyclyl, and heterocyclic ring may be used interchangeably and refer to a stable 5 to 7-membered monocyclic or 6 to 14-membered bicyclic heterocyclic moiety that is either saturated or partially unsaturated, and having, in addition to carbon atoms, one or more, preferably one to four, heteroatoms, as defined above. A heterocyclic ring can be attached to its pendant group at any heteroatom or carbon atom that results in a stable structure and any of the ring atoms can be optionally substituted. Examples of saturated or partially unsaturated heterocyclic rings include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, oxazepinyl, thiazepinyl, and morpholinyl. A heterocyclyl may include groups in a heterocyclyl ring is fused to one or more aryl, heteroaryl, or cycloaliphatic rings, where the radical or point of attachment is on the heterocyclyl ring. A heterocyclyl group may be a mono- or bicyclic.

Partially unsaturated may refer to a ring moiety that includes at least one double or triple bond. It may encompass rings that have multiple sites of unsaturation, but it not intended to include aryl or heteroaryl moieties. The term unsaturated may refer to a moiety that has one or more units of unsaturation.

An aryl may refer to an aryl alone or a larger moiety such as an aralkyl, aralkoxy, and/or aryloxyalkyl. It may refer to monocyclic and bicyclic rings. At least one ring in the system may be aromatic. Each ring in the system may contain 3-7 members. Exemplary aryl groups include, without limitation, phenyl, biphenyl, naphthyl, anthracyl, and the like, which optionally includes one or more substituents. An aryl may also refer to a group in which an aromatic ring is fused to one or more non-aromatic rings such as indanyl, phthalimidyl, naphthimidyl, phenanthridinyl, tetrahydronaphthyl, and the like.

A heteroaryl may refer to a heteroaryl alone or as a part of a larger moiety such as heteroaralkyl or heteroaralkoxy. It may refer to a group having 5-14 ring atoms, preferably 5 or 6 ring atoms. In addition to carbon atoms, the heteroaryl may include 1 to 5 heteroatoms as provided above. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. Heteroaryl may also include groups in which a heteroaromatic ring is fused to one or more aryl, cycloaliphatic, and/or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group is optionally mono- or bicyclic. A heteroaralkyl may refer to an alkyl group substituted by a heteroaryl, wherein the alkyl and heteroaryl portions independently are optionally substituted.

The term optionally substituted may refer to one or more hydrogens of the designated moiety being replaced with a suitable substituent. Unless otherwise indicated, optionally substituted group has suitable substituent at each substitutable position of the group and when more than one position in any given structure is substituted with more than one substituent selected from the specified group, the substituent is either the same or different at every position.

The term stable may refer to compounds that are not substantially altered when subjected to conditions to allow for their production or synthesis, detection, recovery, purification, and/or use as disclosed herein. A pharmaceutically acceptable salt may refer to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds disclosed herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts may be derived from appropriate bases include alkali metal, alkaline earth metal, and ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl sulfonate, and aryl sulfonate.

Unless otherwise stated, structures depicted herein are also meant to include all isomeric (e.g., enantiomeric, diastereomeric, and geometric (or conformational)) forms of the structure. For example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. In some instances, the enantiomeric excess is at least 50%, at least 60%, at least 70%, at least 85%, at least 90%, at least 95%, at least 97%, at least 99%, or 100%. Unless otherwise stated, all tautomeric forms of the compounds disclosed herein are within the scope of the invention. Since the pharmaceutical activity of the racemates or stereoisomers of the compounds according to the invention may differ, it may be desirable to use the enantiomer. In these cases, the end product or even the intermediates can be separated into enantiomeric compounds by chemical or physical measures known to the person skilled in the art or even employed as such in the synthesis.

Where tautomerism, e.g., keto-enol tautomerism, of compounds disclosed herein or their prodrugs may occur, the individual forms, e.g., the keto or the enol form, are claimed separately and together as mixtures in any ratio. The same applies for stereoisomers, e.g., enantiomers, cis/trans isomers, conformers, and the like. If desired, isomers can be separated by methods well known in the art, e.g., by liquid chromatography. The same applies for enantiomers, e.g., by using chiral stationary phases. Additionally, an enantiomer may be isolated by converting it into a diastereomer, i.e., coupling with an enantiomerically pure auxiliary compound, subsequent separation of the resulting diastereomer and cleavage of the auxiliary residue. Alternatively, any enantiomer of a compound disclosed herein may be obtained from stereoselective synthesis using optically pure starting materials.

It is also contemplated that compounds disclosed herein may include isotope-labeled forms thereof. An isotope-labeled form of a compound disclosed herein is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and can be incorporated into a compound of the formula (I) or (II) by well-known methods include, without limitation, isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P³⁵, ¹⁸F, and ³⁶Cl, respectively. It is also contemplated that a compound of the formula (I) or (II), a prodrug thereof, or a pharmaceutically acceptable salt of either which contains one or more of the above-mentioned isotopes and/or other isotopes of other atoms are embodiments of the present disclosure. An isotope-labeled compound of the formula (I) or (II) can be used in a number of beneficial ways. For example, an isotope-labeled compound of the formula (I) or (II) into which, for example, a radioisotope, such as ³H or ¹⁴C, has been incorporated, is suitable for medicament and/or substrate tissue distribution assays. These radioisotopes are particularly preferred due to their ease of preparation and excellent detectability. Incorporation of heavier isotopes, for example, deuterium (²H), into a compound of the formula (I) or (II) may have therapeutic advantages due to the higher metabolic stability of this isotope-labeled compound. Higher metabolic stability translates directly into an increased in vivo half-life or lower dosages. An isotope-labeled compound of the formula (I) or (II) can be adapted to the procedures disclosed in the synthesis schemes and the related description, in the example part and in the preparation part, disclosed herein, replacing a non-isotope-labeled reactant by a readily available isotope-labeled reactant.

The compounds disclosed herein can be in the form of a prodrug compound. A prodrug compound may refer to a derivative that is converted into a biologically active compound under physiological conditions in the living body, e.g., by oxidation, reduction, hydrolysis, or the like, each of which is carried out enzymatically, or without enzyme involvement. Examples of prodrugs are compounds, wherein the amino group in a compound is acylated, alkylated, or phosphorylated; wherein the hydroxyl group is acylated, alkylated, phosphorylated, or converted into borate; wherein the carboxyl group is esterified or amidated; or wherein a sulfhydryl group forms a disulfide bridge with a carrier molecule, e.g., a peptide, that delivers the drug selectively to a target and/or to the cytosol of a cell. These compounds can be produced from compounds disclosed herein according to well-known methods. Other examples of prodrugs are compounds, wherein the carboxylate in a compound is for example converted into an alkyl-, aryl-, choline-, amino, acyloxymethylester, or linolenoyl-ester.

According to an embodiment, a composition comprising a compound disclosed herein or a pharmaceutically acceptable derivative thereof and a pharmaceutically acceptable carrier, adjuvant, or vehicle. The amount of compound in compositions disclosed herein may be effective to measurably inhibit HPK1, or a mutant thereof, in a biological sample or in a patient. A therapeutically effective amount of the compound may be administered to a patient in need thereof. A patient or subject may refer to an animal, preferably a mammal, and even more preferably, a human. A biological sample may refer to, without limitation, cell cultures or extracts thereof; biopsied material obtained from a mammal or extracts thereof; and blood, saliva, urine, feces, semen, tears, or other body fluids or extracts thereof.

A pharmaceutically acceptable carrier, adjuvant, or vehicle may refer to a nontoxic carrier, an adjuvant, or a vehicle that does not destroy the pharmacological activity of the compound with which it is formulated. Pharmaceutically acceptable carriers, adjuvants, or vehicles that are used in the compositions disclosed herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. A pharmaceutically acceptable derivative means any nontoxic salt, ester, salt of an ester or other derivative of a compound disclosed herein that, upon administration to a recipient, is capable of providing, either directly or indirectly, a compound disclosed herein.

Compositions disclosed herein may be administered orally, parenterally, by inhalation spray, topically, rectally, nasally, buccally, vaginally, or via an implanted reservoir. Parenteral administration may refer to subcutaneous, intravenous, intramuscular, intra-articular, intra-synovial, intrasternal, intrathecal, intrahepatic, intralesional, and intracranial injection or infusion techniques. Preferably, the composition may be administered orally, intraperitoneally, or intravenously. Sterile injectable forms of the compositions disclosed herein include aqueous or oleaginous suspension. These suspensions are formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be used are water, Ringer's solution, and isotonic sodium chloride solution. In addition, sterile, fixed oils may be conventionally employed as a solvent or suspending medium.

Pharmaceutically acceptable compositions disclosed herein may be orally administered in any orally acceptable dosage form. Exemplary oral dosage forms are capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried corn starch. When aqueous suspensions are required for oral use, the active ingredient may be combined with emulsifying and suspending agents. If desired, certain sweetening, flavoring, and/or coloring agents may be optionally added. Most preferably, pharmaceutically acceptable compositions disclosed herein are formulated for oral administration. Such formulations may be administered with or without food.

An amount of the compounds disclosed herein that may be combined with the carrier materials to produce a composition in a single dosage form may vary depending upon the host treated, the particular mode of administration. Preferably, provided compositions may be formulated so that a dosage of between 0.01-100 mg/kg body weight/day of the compound can be administered to a patient receiving these compositions. In some instances, the dosage is between 0.1 to 100 mg/kg, 0.1 to 50 mg/kg, 0.5 to 50 mg/kg, 1 to 50 mg/kg, 1 to 20 mg/kg, 5 to 20 mg/kg, 10 to 20 mg/kg, 10-50 mg/kg of body weight of the patient, or 10-100 mg/kg of body weight of the patient or any other dosing range disclosed herein. In some instances, the compounds or compositions herein are administered continuously, multiple times daily, once daily, once every other day, weekly, bi-weekly, monthly, or bi-monthly. A therapeutically effective amount of a compound or composition herein may vary according to factors known in the art, but a dose of about 0.1 to 100 mg/kg, 0.1 to 50 mg/kg, 0.5-50 mg/kg, 1 to 50 mg/kg, 1 to 20 mg/kg, 5 to 20 mg/kg, 10 to 20 mg/kg, or 10-50 mg/kg of body weight of the patient, or 10-100 mg/kg of body weight of the patient or any other dosing range disclosed herein, may be therapeutically effective. A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound disclosed herein in the composition will also depend upon the particular compound in the composition.

The compounds of formula (I) or (II) disclosed herein can be administered before or following an onset of disease once or several times acting as therapy. The aforementioned compounds and medical products of the inventive use are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g., in order to boost the response and eradicate symptoms of the disease completely. Either the identical compound or different compounds can be applied. The methods disclosed herein can also be used to reduce the likelihood of developing a disorder or even prevent the initiation of disorders associated with HPK1 activity in advance or to treat the arising and continuing symptoms.

The host or patient can belong to any mammalian species, for example a primate species, particularly humans; rodents, including mice, rats, and hamsters; rabbits; horses, cows, dogs, cats, etc. Animal models are of interest for experimental investigations, providing a model for treatment of human disease.

The compounds disclosed herein may be useful as anticancer agents for cancers that are responsive to HPK1 inhibition. In certain embodiments, the cancer may include, without limitation, cancer of the breast, bladder, bone, brain, central and peripheral nervous system, colon, endocrine glands, esophagus, endometrium, germ cells, head and neck, kidney, liver, lung, larynx and hypopharynx, ovary, pancreas, prostate, rectum, renal, small intestine, soft tissue, testis, stomach, skin, ureter, vagina, and vulva. In some instances, the cancer is a mesothelioma, sarcoma, retinoblastoma, Wilms tumor, leukemia, lymphoma, non-Hodgkin disease, chronic and acute myeloid leukemia, acute lymphoblastic leukemia, Hodgkin disease, multiple myeloma, T-cell lymphoma, myelodysplastic syndrome, plasma cell neoplasia, and paraneoplastic syndromes.

Sensitivity of a given cancer to HPK1 inhibition can be assessed by, without limitation, measurement of a decrease in primary or metastatic tumor load (minor, partial, or complete regression), alterations in the hemogram, altered hormone or cytokine concentrations in the blood, inhibition of further increase of tumor load, stabilization of the disease in the patient, assessment of biomarkers or surrogate markers relevant for the disease, prolonged overall survival of a patient, prolonged time to disease progression of a patient, prolonged progression-free survival of a patient, prolonged disease-free survival of a patient, improved quality of life of a patient, or modulation of the co-morbidity of the disease (for example, but not limited to, pain, cachexia, mobilization, hospitalization, altered hemogram, weight loss, wound healing, fever). Significant improvements in the pharmacokinetic profiles of compounds of the formula (I) or (ii) may thereby be obtained, and can be expressed quantitatively in terms of increases in the in vivo half-life (t/2), concentration at maximum therapeutic effect (Cmax) and area under the dose response curve (AUC).

In various embodiments, compounds of formula (I) or (II) and related formulae, exhibit an IC₅₀ for the inhibiting HPK1 of less than about 1000 nM, less than about 500 nM, less than 100 nM, less than 500 nM, or less than 1000 nM. In some embodiments, the compounds of formula (I) and (II), and related formulae exhibit an IC₅₀ for the inhibiting HPK1 of at least 1 nM, at least 10 nM, at least 100 nM, or at least 500 nM. In some embodiments, the range is a combination of these values.

Compounds of formula (I) or (II) and/or a physiologically acceptable salt thereof can be employed as an intermediate for the preparation of further medicament active ingredients. The medicament is preferably prepared in a non-chemical manner, e.g., by combining the active ingredient with at least one solid, fluid and/or semi-fluid carrier or excipient, and optionally in conjunction with a single or more other active substances in an appropriate dosage form.

A medicament is provided herein that can include at least one compound disclosed herein or a prodrug or pharmaceutically acceptable salt thereof including mixtures thereof in all ratios. A medicament may refer to any agent in the field of medicine, which comprises one or more compounds of formula (I) or (II) or preparations thereof (e.g., a pharmaceutical composition or pharmaceutical formulation) and can be used in prophylaxis, therapy, follow-up or aftercare of patients who suffer from diseases, which are associated with HPK1 activity, in such a way that a pathogenic modification of their overall condition or of the condition of particular regions of the organism could establish at least temporarily.

In various embodiments, the active ingredient may be administered alone or in combination with other treatments. A synergistic effect may be achieved by using more than one compound in the pharmaceutical composition, i.e. the compound of formula (I) or (II) may be combined with at least another agent as active ingredient, which is either another compound of formula (I) or (II) or a compound of different structural scaffold. The active ingredients can be used either simultaneously or sequentially. In some embodiments, the HPK1 inhibitor compounds disclosed herein is administered simultaneously with one or more additional therapeutic agents. In some embodiments, sequential administration includes administering the HPK1 inhibitor or additional therapeutic agent followed within about any of one minute, five minutes, 30 minutes, one hour, five hours, 24 hours, 48 hours, or a week. In some embodiments, the HPK1 inhibitor is administered by the same route of administration as the additional therapeutic agent. In some embodiments, the HPK1 inhibitor may be administered by a different route of administration than the additional therapeutic agent.

A therapeutic agent may include, without limitation, an anti-inflammatory drug and/or one or more anti-tumor agents conventionally used in chemotherapy or targeted therapy. The compounds or compositions disclosed herein may be used as a monotherapy or may be combined with therapeutic agents. Examples of anti-tumor agents include, without limitation, platinum compounds such as carboplatin, cisplatin, picoplatin, and the like; alkylating agents such as altretamine, carmustine, chlorambucil, mitobronitol, apaziquone, palifosfamide, and the like; DNA altering agents such as bisantrene, decitabine, mitoxantrone, procarbazine, and the like; microtubule modifiers such as docetaxel, eribulin, paclitaxel, vinblastine, and the like; topoisomerase inhibitor such as etoposide, razoxane, topotecan, and the like; anticancer antibodies such as bleomycin, mitomycin C, and the like; antimetabolites such as capecitabine, cladribine, and the like; hormones or antagonists such as tamoxifen, dexamethasone, and the like; cytokines such as interferon and the like; antibodies such as pembrolizumab, nivolumab, ipilimumab, cetuximab, and the like.

A method for inhibiting abnormal cell growth in a mammal, preferably a human, or treating a cancer may include administering to the mammal an amount of a compound of formula (I) or (II) disclosed herein, or a prodrug or a pharmaceutically acceptable salt thereof, in combination with radiation therapy, wherein the amounts of the compound, salt, or prodrug, is in combination with the radiation therapy effective in inhibiting abnormal cell growth or treating the cancer or symptoms thereof in the mammal. Techniques for administering radiation therapy are known in the art, and these techniques can be used in the combination therapy described herein.

As used herein, the terms “treatment”, “treat”, and “treating” may refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent or delay their recurrence.

Disclosed herein is a kit that includes separate packs of a therapeutically effective amount of a compound disclosed herein or a physiologically acceptable salt, or prodrug thereof, and optionally, a therapeutically effective amount of a therapeutic agent. The kit may include suitable containers, such as boxes, individual bottles, bags, or ampoules as well as instructions for using or applying the kit. The kit may, for example, contain separate ampoules, each containing a therapeutically effective amount of a compound disclosed herein and/or pharmaceutically usable derivatives, solvates and stereoisomers thereof, including mixtures thereof in all ratios, and an effective amount of a further therapeutic agent in dissolved or lyophilized form.

### Experimental Procedures

As depicted in the Examples below, in certain exemplary embodiments, compounds are prepared according to the following general procedures. It will be appreciated that, although the general methods depict the synthesis of certain compounds, the following general methods, and other methods known to one of ordinary skill in the art, can be applied to all compounds and subclasses and species of each of these compounds, as described herein.

The symbols and conventions used in the following descriptions of processes, schemes, and examples are consistent with those used in the contemporary scientific literature, for example, the Journal of the American Chemical Society or the Journal of Biological Chemistry.

Unless otherwise indicated, all temperatures are expressed in ° C. (degrees Centigrade).

All solvents used were commercially available and were used without further purification. Reactions were typically run using anhydrous solvents under an inert atmosphere of nitrogen. Flash column chromatography was generally carried out using Silica gel 60 (0.035-0.070 mm particle size).

All NMR experiments were recorded either on Bruker Mercury Plus 400 NMR Spectrometer equipped with a Bruker 400 BBFO probe at 400 MHz for proton NMR, or on Bruker Mercury Plus 300 NMR Spectrometer equipped with a Bruker 300 BBFO probe at 300 MHz for proton NMR, or on a Bruker Avance III 400 NMR Spectrometer equipped with a Bruker PABBO BB-1H/D Z GRD probe at 400 MHz for proton NMR. Most deuterated solvents contained typically 0.03% to 0.05% v/v tetramethylsilane, which was used as the reference signal (set at d 0.00 for both ¹H and ¹³C). In cases where the deuterated solvents did not contain tetramethylsilane, the residual non-deuterated solvent peaks were used as a reference signal, as per published guidelines (J. Org. Chem., Vol. 62, No. 21, 1997).

LC-MS analyses were performed one either one of the two following instruments:
1. SHIMADZU LC-MS machine consisting of an UFLC 20-AD system and LCMS 2020 MS detector. The column used was a Shim-pack XR-ODS, 2.2 μm, 3.0×50 mm. A linear gradient was applied, starting at 95% A (A: 0.05% trifluoroacetic acid (TFA) in water) and ending at 100% B (B: 0.05% TFA in acetonitrile) over 2.2 min with a total run time of 3.6 min. The column temperature was at 40° C. with the flow rate at 1.0 mL/min. The Diode Array detector was scanned from 200-400 nm. The mass spectrometer was equipped with an electro spray ion source (ES) operated in a positive or negative mode. The mass spectrometer was scanned between m/z 90-900 with a scan time of 0.6 s.
2. Agilent 1200 Series mass spectrometers from Agilent Technologies, using either Atmospheric Chemical Ionization (APCI) or Electrospray Ionization (ESI). Diode Array detector was scanned from 200-400 nm. The mass spectrometer was scanned between m/z 90-900 with a scan time of 0.6 s. Column: XBridge C8, 3.5 μm, 4.6×50 mm; Solvent A: water+0.1% TFA; Solvent B: ACN [DEFINE]+0.1% TFA; Flow: 2 ml/min; Gradient: 0 min: 5% B, 8 min: 100% B, 8.1 min: 100% B, 8.5 min: 5% B, 10 min 5% B or a LC/MS Waters ZMD (ESI).

HPLC data were either obtained from the SHIMAZU LC-MS machine or using Agilent 1100 series HPLC from Agilent technologies using a column (XBridge C8, 3.5 μm, 4.6×50 mm) and two mobile phases (mobile phase A: water+0.1% TFA; mobile phase B: ACN+0.1% TFA). The flow rate was 2 ml/min. The gradient method was: 0 min: 5% B; 8 min: 100% B; 8.1 min: 100% B; 8.5 min: 5% B; 10 min 5% B, unless otherwise indicated.

In general, the compounds according to Formula (I) or (II) and related formulae described herein can be prepared from readily available starting materials. If such starting materials are not commercially available, they may be prepared by standard synthetic techniques. In general, the synthesis pathways for any individual compound of Formula (I) or (II) and related formulae will depend on the specific substituents of each molecule, such factors being appreciated by those of ordinary skilled in the art. The following general methods and procedures described hereinafter in the examples may be employed to prepare compounds of Formula (I) or (II) and related formulae. Reaction conditions depicted in the following schemes, such as temperatures, solvents, or co-reagents, are given as examples only and are not restrictive. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures. For all the protection and deprotection methods, see Philip J. Kocienski, “Protecting Groups”, Georg Thieme Verlag Stuttgart, New York, 1994 and, Theodora W. Greene and Peter G. M. Wuts, “Protective Groups in Organic Synthesis”, Wiley Interscience, 3^{rd} Edition 1999.

### Example 1: Synthesis of Compound 1 6-(2-cyano-4-(2-hydroxy-2-methylpropoxy)phenyl)-4-(((3S,5S)-5-fluoropiperidin-3-yl)amino)pyrido[3,2-d]pyrimidine-8-carboxamide

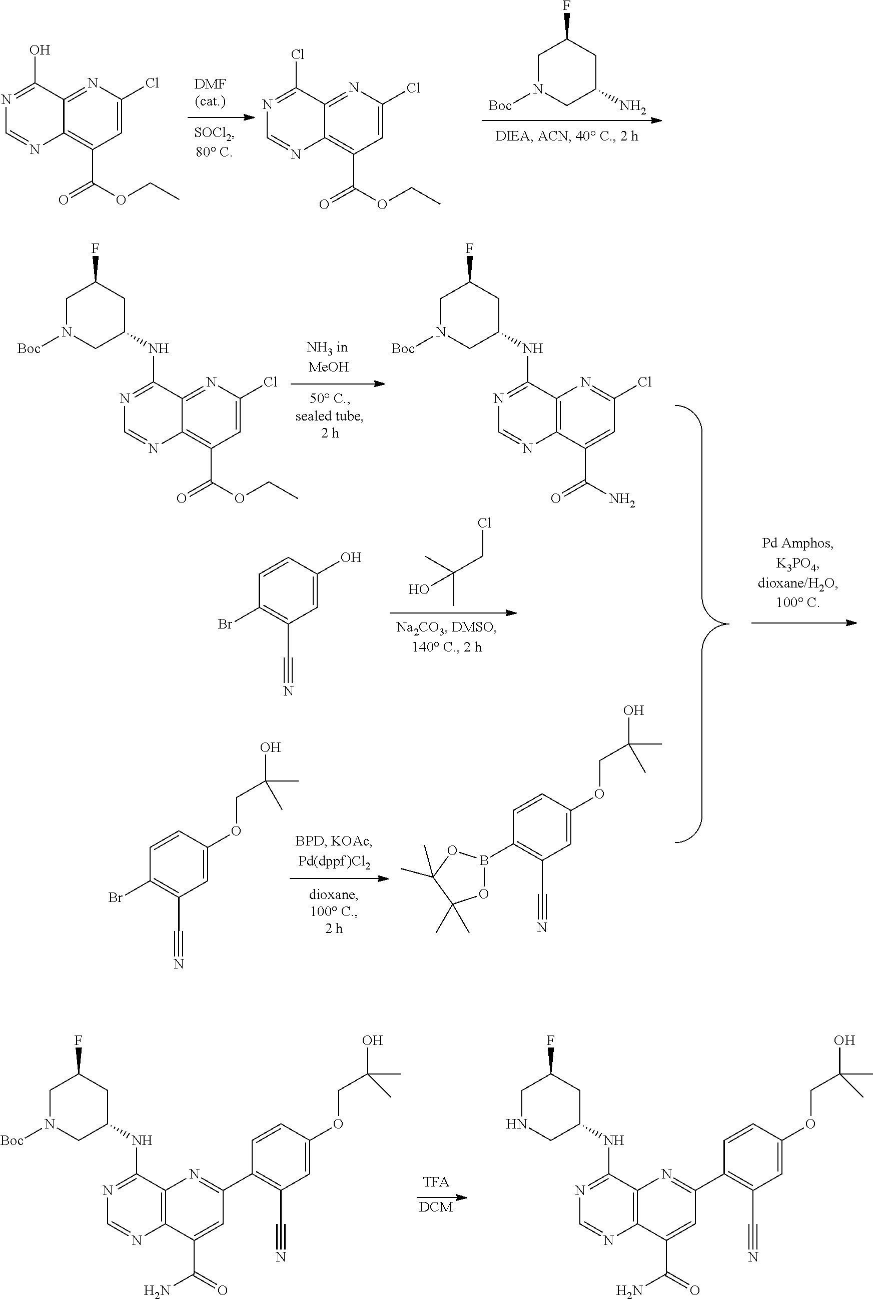

ethyl 4,6-dichloropyrido[3,2-d]pyrimidine-8-carboxylate: To a stirred mixture of methyl 6-chloro-4-oxo-3H,4H-pyrido[3,2-d]pyrimidine-8-carboxylate (500.00 mg; 1.71 mmol) in SOCl₂ (5.00 ml; 66.02 mmol) was added 3 drops of DMF (Dimethylformamide) at room temperature. The resulting mixture was stirred for 4 h (at this time the mixture is a clear solution) at 80° C. under nitrogen atmosphere. The resulting mixture was concentrated under reduced pressure and co-evaporated with DCM 4 times to afford methyl 4,6-dichloropyrido[3,2-d]pyrimidine-8-carboxylate (520.00 mg; crude product) as a brown solid. The residue was used for next step directly without further purification.

ethyl 4-(((3S,5S)-1-(tert-butoxycarbonyl)-5-fluoropiperidin-3-yl)amino)-6-chloropyrido[3,2-d]pyrimidine-8-carboxylate: To a stirred mixture of methyl 4,6-dichloropyrido[3,2-d]pyrimidine-8-carboxylate (500.00 mg; 1.55 mmol) and tert-butyl (3S,5S)-3-amino-5-fluoropiperidine-1-carboxylate (466.00 mg; 2.03 mmol) in MeCN (7.00 ml) was added DIEA [N,N-Diisopropylethylamine](1.01 ml; 5.53 mmol) dropwise at room temperature. The resulting mixture was stirred for 1 h at 40° C. under nitrogen atmosphere. The resulting mixture was added 50 ml water and extracted with EA [Ethyl acetate](3×30 mL). The combined organic layers were washed with brine, dried over anhydrous Na₂SO₄. After filtration, the filtrate was concentrated under reduced pressure to afford tert-butyl (3S,5S)-3-{[6-chloro-8-(methoxycarbonyl) pyrido[3,2-d]pyrimidin-4-yl]amino}-5-fluoropiperidine-1-carboxylate (800.00 mg; crude product) as brown solid.

tert-butyl (3S,5S)-3-((8-carbamoyl-6-chloropyrido[3,2-d]pyrimidin-4-yl)amino)-5-fluoropiperidine-1-carboxylate: A stirred mixture of tert-butyl (3S,5S)-3-{[6-chloro-8-(methoxycarbonyl) pyrido[3,2-d]pyrimidin-4-yl]amino}-5-fluoropiperidine-1-carboxylate (750.00 mg; 1.45 mmol) in NH₃ in MeOH (10.00 ml) was stirred for 1 h at 40° C. The resulting mixture was concentrated under reduced pressure to afford tert-butyl (3S,5S)-3-({8-carbamoyl-6-chloropyrido[3,2-d]pyrimidin-4-yl}amino)-5-fluoropiperidine-1-carboxylate (740.00 mg; crude product) as a brown solid.

2-bromo-5-(2-hydroxy-2-methylpropoxy)benzonitrile: To a stirred mixture of 2-bromo-5-hydroxybenzonitrile (2.00 g; 9.90 mmol) and 1-chloro-2-methylpropan-2-ol (2.26 g; 19.80 mmol) in DMSO (15.00 ml) was added Na₂CO₃ (2.25 g; 19.80 mmol) at room temperature. The resulting mixture was stirred for 16 h at 140° C. The resulting mixture was quenched with water (100 ml) and extracted with EtOAc (100 ml*3). The combined organic layers were washed with brine, dried over anhydrous Na₂SO₄. After filtration, the filtrate was concentrated under reduced pressure. The residue was purified by silica gel column chromatography, eluted with PE/EtOAc (1:1) to afford 2-bromo-5-(2-hydroxy-2-methylpropoxy)benzonitrile as transparent oil (2.50 g 84.15%).

5-(2-hydroxy-2-methylpropoxy)-2-(4,4,5,5-tetramethyl-1,3,2-dioxaborolan-2-yl)benzonitrile: To a stirred mixture of 2-bromo-5-(2-hydroxy-2-methylpropoxy)benzonitrile (1.50 g; 5.00 mmol) and BPD (2.67 g; 10.00 mmol) in 1,4-Dioxane (25.00 ml) were added Pd(dppf)Cl₂ [(1,1′-Bis(diphenylphosphino)ferrocene)palladium(II) dichloride](0.38 g; 0.50 mmol) and AcOK (1.49 g; 14.99 mmol) at room temperature. The resulting mixture was stirred for 2 h at 100 degrees C. under argon atmosphere. The solvent was removed under vacuum, the residue was purified by silica gel column chromatography, eluted with PE/EtOAc (1:1) to afford 5-(2-hydroxy-2-methylpropoxy)-2-(4,4,5,5-tetramethyl-1,3,2-dioxaborolan-2-yl)benzonitrile as brown oil (1.60 g; 79.26%).

tert-butyl (3S,5S)-3-((8-carbamoyl-6-(2-cyano-4-(2-hydroxy-2-methylpropoxy)phenyl)pyrido[3,2-d]pyrimidin-4-yl)amino)-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (120.00 mg; 0.22 mmol) and 5-(2-hydroxy-2-methylpropoxy)-2-(4,4,5,5-tetramethyl-1,3,2-dioxaborolan-2-yl)benzonitrile (133.85 mg; 0.33 mmol) in 1,4-Dioxane (12.00 ml) and H₂O (2.40 ml) were added AMPHOS-PdCl₂ (16.47 mg; 0.02 mmol) and K₃PO₄ (148.09 mg; 0.66 mmol) at room temperature. The resulting mixture was stirred for 2 h at 100° C. under argon atmosphere. The mixture was concentrated under reduced pressure and the residue was purified by silica gel column chromatography, eluted with DCM/MeOH [dichloromethane](6:1) to afford tert-butyl(3S,5S)-3-({8-carbamoyl-6-[2-cyano-4-(2-hydroxy-2-methylpropoxy)phenyl]pyrido[3,2-d]pyrimidin-4-yl}amino)-5-fluoropiperidine-1-carboxylate as yellow solid (120.00 mg, 89.80%).

6-(2-cyano-4-(2-hydroxy-2-methylpropoxy)phenyl)-4-(((3S,5S)-5-fluoropiperidin-3-yl)amino)pyrido[3,2-d]pyrimidine-8-carboxamide: To a stirred mixture of tert-butyl(3S,5S)-3-({8-carbamoyl-6-[2-cyano-4-(2-hydroxy-2-methylpropoxy)phenyl]pyrido[3,2-d]pyrimidin-4-yl}amino)-5-fluoropiperidine-1-carboxylate (110.00 mg; 0.18 mmol) in DCM (6.00 ml) and TFA (1.00 ml) at room temperature. The resulting mixture was stirred for 1 h at room temperature. The mixture was concentrated under reduced pressure. The crude product was purified by Prep-HPLC(Column: YMC-Actus Triart C18 ExRS, 30*150 mm, 5 μm; Mobile Phase A: Water (10 mmol/L NH₄HCO₃+0.1% [28% NH₃ in H₂O]), Mobile Phase B: ACN; Flow rate: 60 mL/min; Gradient: 18% B to 48% B in 8 min, 48% B; Wave Length: 254 nm; RT1 (min): 7; Number Of Runs: 5) to afford 6-[2-cyano-4-(2-hydroxy-2-methylpropoxy)phenyl]-4-{[(3S,5S)-5-fluoropiperidin-3-yl]amino}pyrido[3,2-d]pyrimidine-8-carboxamide as yellow solid (46.00 mg, 51.99%).

HPLC: 98.94% purity, RT=2.85 min. MS: m/z=480.2 [M+H]⁺. ¹H NMR (300 MHz, DMSO-d6, ppm): δ 9.92 (d, J=3.6 Hz, 1H), 8.79 (s, 1H), 8.61 (s, 1H), 8.29-8.17 (m, 2H), 7.89 (d, J=8.5 Hz, 1H), 7.57 (d, J=2.7 Hz, 1H), 7.41 (dd, J=8.9, 2.7 Hz, 1H), 4.99-4.64 (m, 2H), 4.50 (s, 1H), 3.89 (s, 2H), 3.17-2.81 (m, 2H), 2.75-2.53 (m, 2H), 2.22 (s, 1H), 1.95 (dt, J=35.7, 12.0 Hz, 1H), 1.22 (s, 6H).

### Example 2: Synthesis of Compound 2 4-{[(3S,5S)-5-fluoropiperidin-3-yl]amino}-6-(3-methyl-1,2-thiazol-5-yl)pyrido[3,2-d]pyrimidine-8-carboxamide

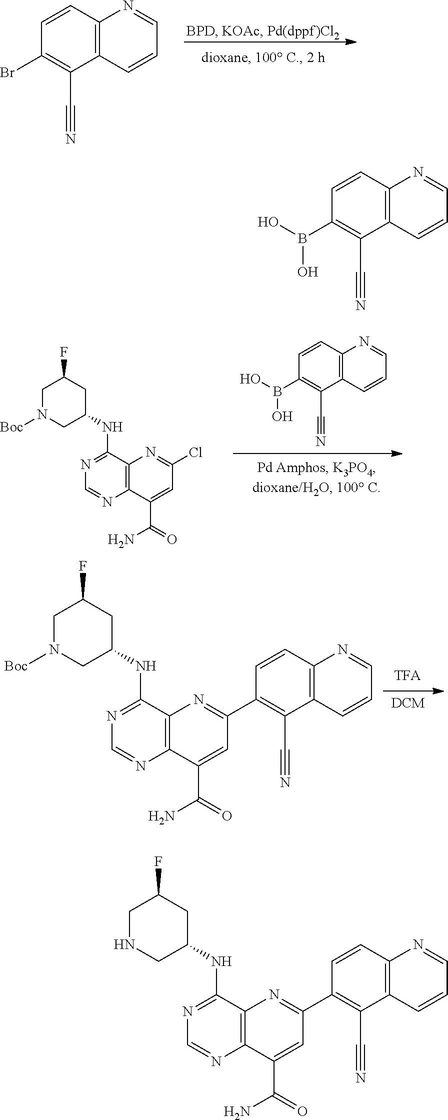

(5-cyanoquinolin-6-yl)boronic acid: To a stirred mixture of 6-bromoquinoline-5-carbonitrile (230.00 mg; 0.94 mmol) and BPD (794.00 mg; 2.81 mmol) in 1,4-Dioxane (8.00 ml) were added Pd(dppf)Cl₂ (72.00 mg; 0.09 mmol) and 1,4-Dioxane (8.00 ml) in portions at 25° C. The resulting mixture was stirred for 2 h at 100° C. under nitrogen atmosphere. The reaction was quenched with H₂O at 25° C. The resulting mixture was extracted with EA (3×100 mL). The combined organic layers were washed with brine, dried over anhydrous MgSO₄. After filtration, the filtrate was concentrated under reduced pressure. The residue was purified by silica gel column chromatography, eluted with PE:EA=80:20 to afford (5-cyanoquinolin-6-yl) boronic acid (200.00 mg; 94.9%) as a white solid.

tert-butyl (3S,5S)-3-{[8-carbamoyl-6-(5-cyanoquinolin-6-yl)pyrido[3,2-d]pyrimidin-4-yl]amino}-5-fluoropiperidine-1-carboxylate: To a stirred mixture of (5-cyanoquinolin-6-yl) boronic acid (186.00 mg; 0.83 mmol) and tert-butyl (3S,5S)-3-({8-carbamoyl-6-chloropyrido[3,2-d]pyrimidin-4-yl}amino)-5-fluoropiperidine-1-carboxylate (150.00 mg; 0.28 mmol) in 1,4-Dioxane (8.00 ml) were added AMPHOS-PdCl₂ (21.00 mg; 0.03 mmol;), K₃PO₄ (185.00 mg; 0.83 mmol) and H₂O (2.00 ml) in portions at 25° C. The resulting mixture was stirred for 2 h at 100° C. under nitrogen atmosphere. The resulting mixture was concentrated under vacuum. The residue was purified by silica gel column chromatography, eluted with PE:EA=20:80 to afford tert-butyl (3S,5S)-3-{[8-carbamoyl-6-(5-cyanoquinolin-6-yl)pyrido[3,2-d]pyrimidin-4-yl]amino}-5-fluoropiperidine-1-carboxylate (120.00 mg; 67.3%) as a yellow solid.

6-(5-cyanoquinolin-6-yl)-4-{[(3S,5S)-5-fluoropiperidin-3-yl]amino}pyrido[3,2-d]pyrimidine-8-carboxamide: To a stirred mixture of tert-butyl (3S,5S)-3-{[8-carbamoyl-6-(5-cyanoquinolin-6-yl)pyrido[3,2-d]pyrimidin-4-yl]amino}-5-fluoropiperidine-1-carboxylate (100.00 mg; 0.15 mmol) in DCM (8.00 ml) was added TFA (2.00 ml) dropwise at 25° C. The resulting mixture was stirred for 2 h at 25° C. under nitrogen atmosphere. The resulting mixture was concentrated under vacuum. The crude product was purified by Prep-HPLC with the following conditions Column: Xselect CSH C18 OBD Column 30*150 mm 5 μm, n; Mobile Phase A: Water (0.1% FA [formic acid]), Mobile Phase B: ACN; Flow rate: 60 mL/min.; Gradient: 5% B to 30% B in 8 min., 30% B; Wave Length: 254/220 nm; RT1 (min.): 7.22; Number Of Runs: 2, to afford 6-(5-cyanoquinolin-6-yl)-4-{[(3S,5S)-5-fluoropiperidin-3-yl]amino}pyrido[3,2-d]pyrimidine-8-carboxamide; formic acid (51.30 mg; 62.1%) as a yellow solid.

HPLC: 91.6% purity, RT=2.6 min. MS: m/z=443.1 [M+H]⁺. ¹H NMR (400 MHz, DMSO-d6) δ 9.90 (d, J=3.5 Hz, 1H), 9.17 (dd, J=4.2, 1.6 Hz, 1H), 9.03 (s, 1H), 8.75 (s, 1H), 8.68 (dd, J=8.8, 1.6 Hz, 1H), 8.59 (d, J=8.9 Hz, 1H), 8.52 (d, J=8.7 Hz, 2H), 8.38 (d, J=3.5 Hz, 1H), 7.90 (dd, J=8.6, 4.2 Hz, 1H), 5.31 (d, J=45.0 Hz, 1H), 5.01-4.88 (m, 1H), 3.60 (t, J=12.7 Hz, 1H), 3.53-3.45 (m, 1H), 3.22-3.10 (m, 2), 2.45 (d, J=14.3 Hz, 2H), 2.18 (m, J=44.3, 13.2 Hz, 1H).

### Example 3: Synthesis of Compound 3 6-(1,3-dimethyl-1H-pyrazol-4-yl)-4-(((3S,5S)-5-fluoropiperidin-3-yl)amino)pyrido[3,2-d]pyrimidine-8-carboxamide

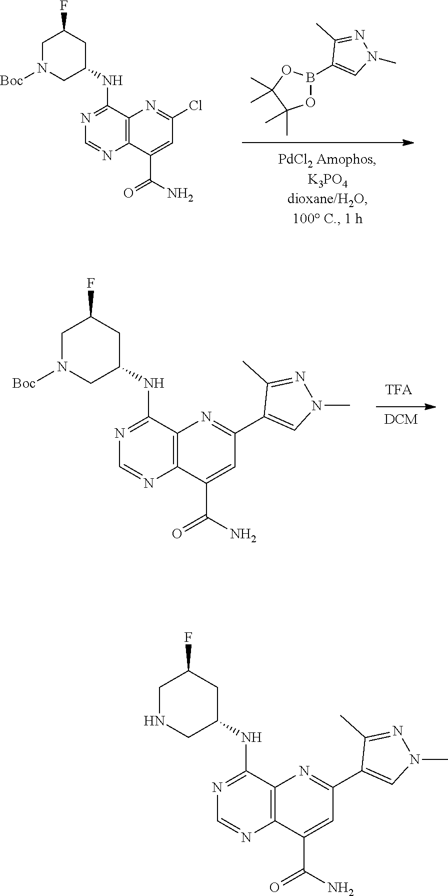

tert-butyl (3S,5S)-3-((8-carbamoyl-6-(1,3-dimethyl-1H-pyrazol-4-yl)pyrido[3,2-d]pyrimidin-4-yl)amino)-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (120.00 mg; 0.22 mmol) and 1,3-dimethyl-4-(4,4,5,5-tetramethyl-1,3,2-dioxaborolan-2-yl)-1H-pyrazole (58.87 mg, 0.27 mmol) in 1,4-Dioxane (5.00 ml) and Water (1.00 ml) were added Bis(di-tert-butyl(4-dimethylaminophenyl)phosphine)dichloropalladium(II) (16.47 mg; 0.02 mmol) and K₃PO₄ (148.05 mg; 0.66 mmol) at room temperature. It was stirred for 12 h at 100 degrees C. under argon atmosphere. The resulting mixture was concentrated under reduced pressure and the residue was purified by silica gel column chromatography, eluted with PE/EA (1:9) to afford tert-butyl (3S,5S)-3-{[8-carbamoyl-6-(1,3-dimethyl-1H-pyrazol-4-yl)pyrido[3,2-d]pyrimidin-4-yl]amino}-5-fluoropiperidine-1-carboxylate as yellow solid (90.00 mg; 67.4%).

6-(1,3-dimethyl-1H-pyrazol-4-yl)-4-(((3S,5S)-5-fluoropiperidin-3-yl)amino)pyrido[3,2-d]pyrimidine-8-carboxamide: To a solution of tert-butyl (3S,5S)-3-{[8-carbamoyl-6-(1,3-dimethyl-1H-pyrazol-4-yl)pyrido[3,2-d]pyrimidin-4-yl]amino}-5-fluoropiperidine-1-carboxylate (100.00 mg; 0.19 mmol) in DCM (4.00 ml) were added TFA (2.00 ml). After stirring for 2 h at 25° C. The resulting mixture was concentrated under reduced pressure. The residue was purified by Prep-HPLC(Column: XBridge Shield RP18 OBD Column, 30*150 mm, 5 μm; Mobile Phase A: Water (10 mmol/L NH₄HCO₃+0.1% [28% NH₃ in H₂O]), Mobile Phase B: ACN; Flow rate: 60 mL/min.; Gradient: 16% B to 43% B in 9 min., 43% B; Wave Length: 254 nm; RT1 (min.): 7; Number Of Runs: 1) to afford 6-(1,3-dimethyl-1H-pyrazol-4-yl)-4-{[(3S,5S)-5-fluoropiperidin-3-yl]amino}pyrido[3,2-d]pyrimidine-8-carboxamide as off-white solid (51.00 mg; 70.0%).

HPLC: 98.03% purity, RT=2.04 min. MS: m/z=385.10 [M+H]⁺. ¹H NMR (300 MHz, DMSO-d6, ppm): δ 10.43 (d, 1H), 8.77 (d, 1H), 8.63 (d, 1H), 8.55 (d, 2H), 8.24 (d, 1H), 7.89 (d, 1H), 7.38 (s, 2H), 4.88 (d, 1H), 4.62 (s, 1H), 3.13 (s, 1H), 3.03 (dd, 2H), 2.69-2.54 (m, 3H), 2.29 (s, 3H), 2.05 (d, 1H).

### Example 4: Synthesis of Compound 4 4-(((3S,5S)-5-fluoropiperidin-3-yl)amino)-6-(1-(tetrahydro-2H-pyran-4-yl)-1H-pyrazol-4-yl)pyrido[3,2-d]pyrimidine-8-carboxamide

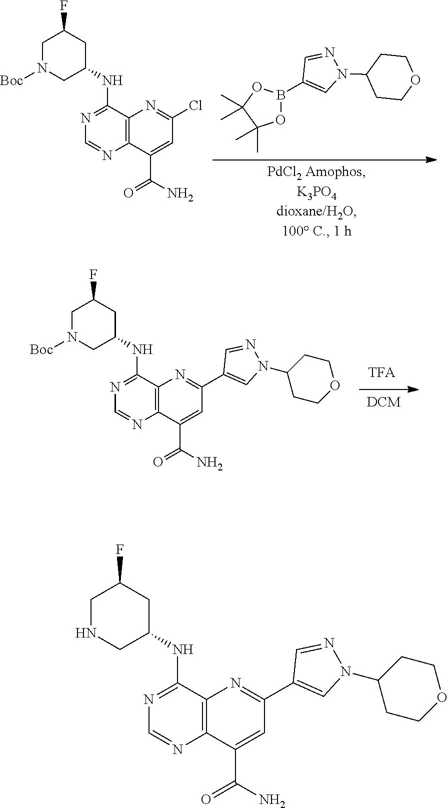

tert-butyl (3S,5S)-3-((8-carbamoyl-6-(1-(tetrahydro-2H-pyran-4-yl)-1H-pyrazol-4-yl)pyrido[3,2-d]pyrimidin-4-yl)amino)-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (120.00 mg; 0.22 mmol) and 1-(oxan-4-yl)-4-(4,4,5,5-tetramethyl-1,3,2-dioxaborolan-2-yl)-1H-pyrazole (94.06 mg; 0.33 mmol) in 1,4-Dioxane (5.00 ml) and water (1.00 ml) were added Bis(di-tert-butyl(4-dimethylaminophenyl)phosphine)dichloropalladium(II) (16.47 mg; 0.02 mmol) and K₃PO₄ (148.05 mg; 0.66 mmol) at room temperature. The resulting mixture was stirred for 2 h at 100 degrees C. under argon atmosphere. The mixture was concentrated under reduced pressure. The residue was purified by silica gel column chromatography, eluted with PE/EtOAc (1:4) to afford tert-butyl (3S,5S)-3-({8-carbamoyl-6-[1-(oxan-4-yl)-1H-pyrazol-4-yl]pyrido[3,2-d]pyrimidin-4-yl}amino)-5-fluoropiperidine-1-carboxylate as yellow solid (110.00 mg; 80.29%).

4-(((3S,5S)-5-fluoropiperidin-3-yl)amino)-6-(1-(tetrahydro-2H-pyran-4-yl)-1H-pyrazol-4-yl)pyrido[3,2-d]pyrimidine-8-carboxamide: To a stirred mixture of tert-butyl(3S,5S)-3-({8-carbamoyl-6-[1-(oxan-4-yl)-1H-pyrazol-4-yl]pyrido[3,2-d]pyrimidin-4-yl}amino)-5-fluoropiperidine-1-carboxylate (110.00 mg; 0.18 mmol) and TFA (1.00 ml) in DCM (6.00 ml). After stirring for 1 h at room temperature. The mixture was concentrated under reduced pressure. The crude product was purified by Prep-HPLC(Column: Xselect CSH C18 OBD Column 30*150 mm 5 μm, n; Mobile Phase A: Water (10 mmol/L NH₄HCO₃+0.1% [28% NH₃ in H₂O]), Mobile Phase B: ACN; Flow rate: 60 mL/min.; Gradient: 7% B to 37% B in 8 min., 37% B; Wave Length: 254/220 nm; RT1 (min.): 8.2; Number Of Runs: 2) to afford 4-{[(3S,5S)-5-fluoropiperidin-3-yl]amino}-6-[1-(oxan-4-yl)-1H-pyrazol-4-yl]pyrido[3,2-d]pyrimidine-8-carboxamide as yellow solid (43.20 mg; 55.14%).

HPLC: 99.74% purity, RT=2.13 min. MS: m/z=441.15 [M+H]⁺. ¹H NMR (300 MHz, DMSO-d6, ppm): δ 9.99 (d, J=3.6 Hz, 1H), 8.79 (s, 1H), 8.57 (d, J=17.0 Hz, 2H), 8.44 (s, 1H), 8.20 (t, J=7.0 Hz, 2H), 5.04-4.88 (s, 1H), 4.63 (s, 1H), 4.49 (tt, J=10.3, 5.2 Hz, 1H), 4.05-3.94 (m, 2H), 3.51 (td, J=11.3, 3.4 Hz, 2H), 3.13 (t, J=13.7 Hz, 2H), 2.94-2.71 (m, 2H), 2.55 (s, 1H), 2.24 (dd, J=15.1, 5.5 Hz, 1H), 2.11-1.92 (m, 4H).

### Example 5: Synthesis of Compound 5 6-(5-acetylthiophen-2-yl)-4-(((3S,5S)-5-fluoropiperidin-3-yl)amino)pyrido[3,2-d]pyrimidine-8-carboxamide

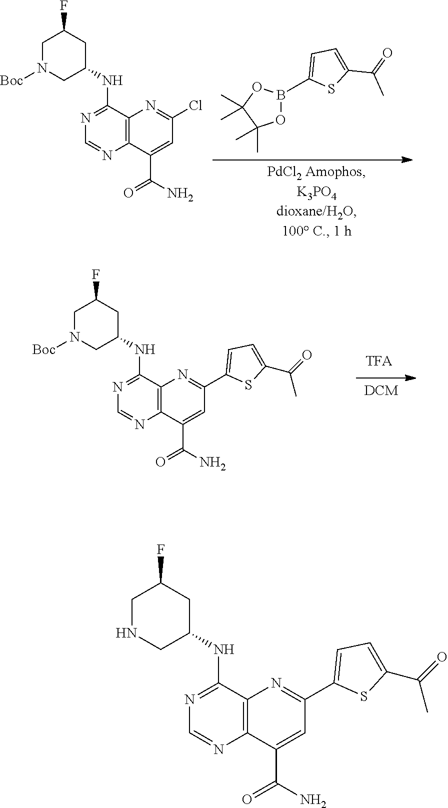

tert-butyl(3S,5S)-3-((6-(5-acetylthiophen-2-yl)-8-carbamoylpyrido[3,2-d]pyrimidin-4-yl)amino)-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (120.00 mg; 0.22 mmol; 1.00 eq.) and (5-acetylthiophen-2-yl)boronic acid (57.49 mg; 0.33 mmol; 1.50 eq.) in 1,4-Dioxane (5.00 ml) and water (1.00 ml) were added Bis(di-tert-butyl(4-dimethylaminophenyl)phosphine)dichloropalladium(II) (16.47 mg; 0.02 mmol) and K₃PO₄ (148.05 mg; 0.66 mmol) at room temperature. The resulting mixture was stirred for 2 h at 100° C. under argon atmosphere. The mixture was concentrated under reduced pressure. The residue was purified by silica gel column chromatography, eluted with PE/EtOAc (1:4) to afford tert-butyl (3S,5S)-3-{[6-(5-acetylthiophen-2-yl)-8-carbamoylpyrido[3,2-d]pyrimidin-4-yl]amino}-5-fluoropiperidine-1-carboxylate as yellow solid (90.00 mg, 64.79%).

6-(5-acetylthiophen-2-yl)-4-(((3S,5S)-5-fluoropiperidin-3-yl)amino)pyrido[3,2-d]pyrimidine-8-carboxamide: To a stirred mixture of tert-butyl (3S,5S)-3-{[6-(5-acetylthiophen-2-yl)-8-carbamoylpyrido[3,2-d]pyrimidin-4-yl]amino}-5-fluoropiperidine-1-carboxylate (90.00 mg; 0.14 mmol) and TFA (1.00 ml) in DCM (6.00 ml). The mixture was stirred for 1 h at room temperature. The mixture was concentrated under reduced pressure, and the crude product was purified by Prep-HPLC(Column: XBridge Shield RP18 OBD Column, 30*150 mm, 5 μm; Mobile Phase A: Water (10 mmol/L NH₄HCO₃+0.1% [28% NH₃ in H₂O]), Mobile Phase B: ACN; Flow rate: 60 mL/min.; Gradient: 19% B to 45% B in 9 min., 45% B; Wave Length: 254 nm; RT1 (min.): 7; Number Of Runs: 2) to afford 6-(5-acetylthiophen-2-yl)-4-{[(3S,5S)-5-fluoropiperidin-3-yl]amino}pyrido[3,2-d]pyrimidine-8-carboxamide as yellow solid (31.60 mg; 53.03%).

HPLC: 99.56% purity, RT=2.57 min. MS: m/z=415.15 [M+H]⁺. ¹H NMR (300 MHz, DMSO-d6, ppm): δ 9.92 (d, J=3.6 Hz, 1H), 8.78 (s, 1H), 8.59 (s, 1H), 8.25-8.12 (m, 3H), 8.00 (dd, J=4.0, 1.2 Hz, 1H), 4.96-4.80 (s, 1H), 4.62 (dt, J=9.7, 4.9 Hz, 1H), 3.13-2.95 (m, 2H), 2.93-2.70 (m, 2H), 2.60 (s, 3H), 2.36-2.17 (m, 2H).

### Example 6: Synthesis of Compound 6 4-{[(3S,5S)-5-fluoropiperidin-3-yl]amino}-6-(3-methyl-1,2-thiazol-5-yl)pyrido[3,2-d]pyrimidine-8-carboxamide

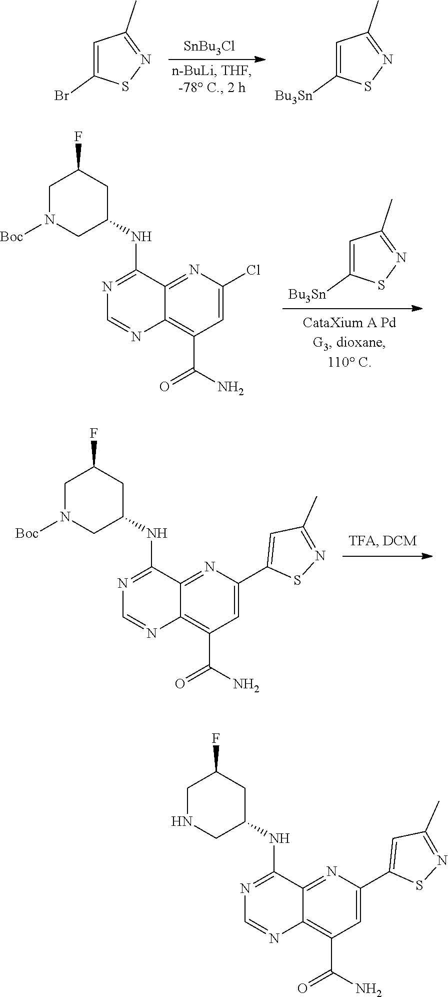

3-methyl-5-(tributylstannyl)-1,2-thiazole: To a stirred solution of 5-bromo-3-methyl-1,2-thiazole (500.00 mg; 2.67 mmol) in THF (5.00 ml) was added n-BuLi (1.20 ml, 2.5M in hexane) at −78° C. under nitrogen atmosphere. The resulting mixture was stirred for 1 h at −78° C. under nitrogen atmosphere. Then tributyl(chloro)stannane (921.00 mg; 2.80 mmol) in THF (2.00 ml) was added dropwise at −78° C. The resulting mixture was stirred for 2 h at −78° C. under nitrogen atmosphere. The reaction was quenched by the addition of sat. NH₄Cl (50 mL) at 0° C. The resulting mixture was extracted with EA (3×40 mL). The combined organic layers were washed with brine, dried over anhydrous Na₂SO₄. After filtration, the filtrate was concentrated under reduced pressure. The residue was purified by silica gel column chromatography, eluted with 10% EA in PE to afford 3-methyl-5-(tributylstannyl)-1,2-thiazole (1.10 g; 2.63 mmol) as a yellow liquid.

tert-butyl (3S,5S)-3-({8-carbamoyl-6-chloropyrido[3,2-d]pyrimidin-4-yl}amino)-5-fluoropiperidine-1-carboxylate: To a stirred solution of tert-butyl (3S,5S)-3-({8-carbamoyl-6-chloropyrido[3,2-d]pyrimidin-4-yl}amino)-5-fluoropiperidine-1-carboxylate (200.00 mg; 0.37 mmol) and 3-methyl-5-(tributylstannyl)-1,2-thiazole (275.00 mg; 0.66 mmol) in 1,4-Dioxane (3.00 ml) was added cataCXium A Pd G₃ (35.00 mg; 0.05 mmol) in portions at room temperature under N₂ atmosphere. The resulting mixture was stirred for 2 h at 100° C. under N₂ atmosphere. The resulting mixture was concentrated under reduced pressure. The residue was purified by silica gel column chromatography, eluted with 45% EA in PE to afford tert-butyl (3S,5S)-3-{[8-carbamoyl-6-(3-methyl-1,2-thiazol-5-yl)pyrido[3,2-d]pyrimidin-4-yl]amino}-5-fluoropiperidine-1-carboxylate (190.00 mg; 98.2%) as a yellow solid.

4-{[(3S,5S)-5-fluoropiperidin-3-yl]amino}-6-(3-methyl-1,2-thiazol-5-yl)pyrido[3,2-d]pyrimidine-8-carboxamide: To a stirred mixture of tert-butyl (3S,5S)-3-{[8-carbamoyl-6-(3-methyl-1,2-thiazol-5-yl)pyrido[3,2-d]pyrimidin-4-yl]amino}-5-fluoropiperidine-1-carboxylate (170.00 mg; 0.32 mmol) in DCM (3.00 ml) was added TFA (1.00 ml) dropwise at 0° C. The resulting mixture was stirred for 1 h at room temperature under nitrogen atmosphere. The resulting mixture was concentrated under reduced pressure. The crude product was purified by Prep-HPLC with the following conditions (Column: XBridge Shield RP18 OBD Column, 30*150 mm, 5 μm; Mobile Phase A: Water (10 mmol/L NH₄HCO₃+0.1% [28% NH₃ in H₂O]), Mobile Phase B: ACN; Flow rate: 60 mL/min.; Gradient: 18% B to 44% B in 9 min., 44% B; Wave Length: 254 nm; RT1 (min): 7) to afford 4-{[(3S,5S)-5-fluoropiperidin-3-yl]amino}-6-(3-methyl-1,2-thiazol-5-yl)pyrido[3,2-d]pyrimidine-8-carboxamide (101.90 mg; 80.4%) as a yellow solid.

HPLC: 98.9% purity, RT=2.39 min. MS: m/z=388.2 [M+H]⁺. ¹H NMR (400 MHz, DMSO-d6) δ 9.94 (d, J=3.5 Hz, 1H), 8.71 (s, 1H), 8.61 (s, 1H), 8.27 (d, J=3.4 Hz, 1H), 8.19 (d, J=8.8 Hz, 1H), 8.10 (s, 1H), 4.86 (d, J=48.0 Hz, 1H), 4.64-4.55 (m, 1H), 3.09-2.93 (m, 2H), 2.85-2.68 (m, 3H), 2.52 (m, 3H), 2.42-2.05 (m, 2H).

### Example 7: Synthesis of Compound 7 6-(5-cyano-1-methyl-1H-pyrrol-3-yl)-4-{[(3S,5S)-5-fluoropiperidin-3-yl]amino}pyrido[3,2-d]pyrimidine-8-carboxamide

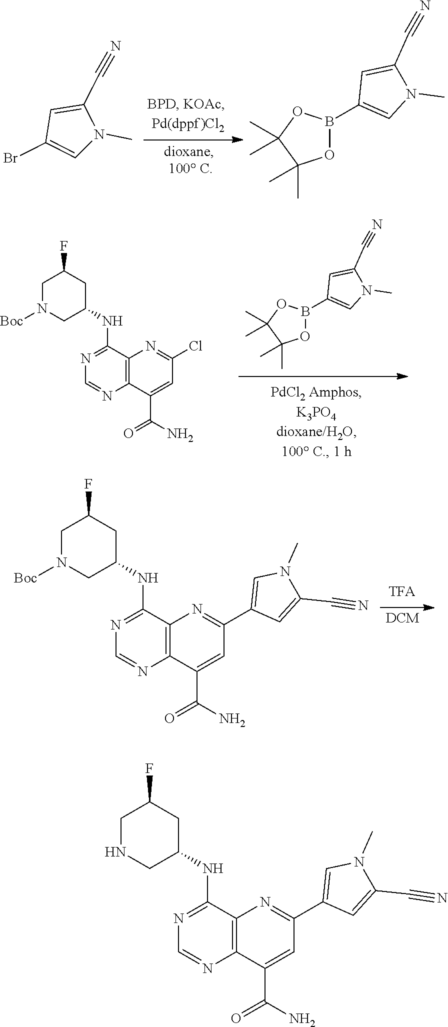

1-methyl-4-(4,4,5,5-tetramethyl-1,3,2-dioxaborolan-2-yl)-1H-pyrrole-2-carbonitrile: A solution of 4-bromo-1-methyl-1H-pyrrole-2-carbonitrile (1.00 g; 3.92 mmol;), Bis(pinacolato) diboron (2.60 g; 9.73 mmol), KOAc (608.00 mg; 5.89 mmol) and Pd(dppf)Cl₂—CH₂Cl₂ (338.00 mg; 0.39 mmol) in 1,4-Dioxane (20.00 ml) was stirred for 16 h at 100° C. under nitrogen atmosphere. The reaction was monitored by LCMS. The mixture was allowed to cool down to room temperature. The resulting mixture was diluted with water (100 mL). The resulting mixture was extracted with EtOAc (3×100 mL). The combined organic layers were washed with brine (1×300 mL), dried over anhydrous Na₂SO₄. After filtration, the filtrate was concentrated under reduced pressure. The residue was purified by silica gel column chromatography, eluted with DCM/MeOH (10:1) to afford 1-methyl-4-(4,4,5,5-tetramethyl-1,3,2-dioxaborolan-2-yl)-1H-pyrrole-2-carbonitrile (500.00 mg; 51.7%) as a yellow solid.

tert-butyl (3S,5S)-3-{[8-carbamoyl-6-(5-cyano-1-methyl-1H-pyrrol-3-yl)pyrido[3,2-d]pyrimidin-4-yl]amino}-5-fluoropiperidine-1-carboxylate: To a stirred solution of tert-butyl (3S,5S)-3-({8-carbamoyl-6-chloropyrido[3,2-d]pyrimidin-4-yl}amino)-5-fluoropiperidine-1-carboxylate (200.00 mg; 0.37 mmol) and 1-methyl-4-(4,4,5,5-tetramethyl-1,3,2-dioxaborolan-2-yl)-1H-pyrrole-2-carbonitrile in 1,4-Dioxane (2.50 ml) and Water (0.25 ml) were added AMPHOS-PdCl₂ (180.00 mg; 0.24 mmol) and potassium phosphate tribasic (31.00 mg; 0.14 mmol) in portions at room temperature under N₂ atmosphere. The resulting mixture was stirred for 2 h at 100° C. under N₂ atmosphere. The resulting mixture was concentrated under reduced pressure. The residue was purified by silica gel column chromatography, eluted with 55% EA in PE to afford tert-butyl (3S,5S)-3-{[8-carbamoyl-6-(5-cyano-1-methyl-1H-pyrrol-3-yl)pyrido[3,2-d]pyrimidin-4-yl]amino}-5-fluoropiperidine-1-carboxylate (200.00 mg; 98.4%) as a yellow solid.

6-(5-cyano-1-methyl-1H-pyrrol-3-yl)-4-{[(3S,5S)-5-fluoropiperidin-3-yl]amino}pyrido[3,2-d]pyrimidine-8-carboxamide: To a stirred mixture of tert-butyl (3S,5S)-3-{[8-carbamoyl-6-(5-cyano-1-methyl-1H-pyrrol-3-yl)pyrido[3,2-d]pyrimidin-4-yl]amino}-5-fluoropiperidine-1-carboxylate (180.00 mg; 0.33 mmol) in DCM (3.00 ml) was added TFA (1.00 ml) dropwise at 0° C. The resulting mixture was stirred for 1 h at room temperature under nitrogen atmosphere. The resulting mixture was concentrated under reduced pressure. The crude product was purified by Prep-HPLC with the following conditions (Column: XBridge Shield RP18 OBD Column, 30*150 mm, 5 μm; Mobile Phase A: Water (10 mmol/L NH₄HCO₃+0.1% [28% NH₃ in H₂O]), Mobile Phase B: ACN; Flow rate: 60 mL/min.; Gradient: 19% B to 44% B in 9 min., 44% B; Wave Length: 254 nm; RT1 (min.): 7; Number Of Runs: 2) to afford 6-(5-cyano-1-methyl-1H-pyrrol-3-yl)-4-{[(3S,5S)-5-fluoropiperidin-3-yl]amino}pyrido[3,2-d]pyrimidine-8-carboxamide (89.60 mg; 68.5%) as a yellow solid.

HPLC: 98.3% purity, RT=2.36 min. MS: m/z=395.0 [M+H]⁺. ¹H NMR (400 MHz, DMSO, ppm) δ 10.02 (d, J=3.6 Hz, 1H), 8.53 (d, J=10.5 Hz, 2H), 8.24 (d, J=1.7 Hz, 1H), 8.22-8.15 (m, 2H), 7.96 (d, J=1.8 Hz, 1H), 4.89 (d, J=48.0 Hz, 1H), 4.58 (m, J=9.9, 5.3, 4.6 Hz, 1H), 3.85 (s, 3H), 3.14-2.61 (m, 4H), 2.51 (p, J=1.8 Hz, 1H), 2.13 (m, 2H).

### Example 8: Synthesis of Compound 8 6-(1-ethyl-5-methyl-1H-pyrazol-4-yl)-4-(((3S,5S)-5-fluoropiperidin-3-yl)amino)pyrido[3,2-d]pyrimidine-8-carboxamide

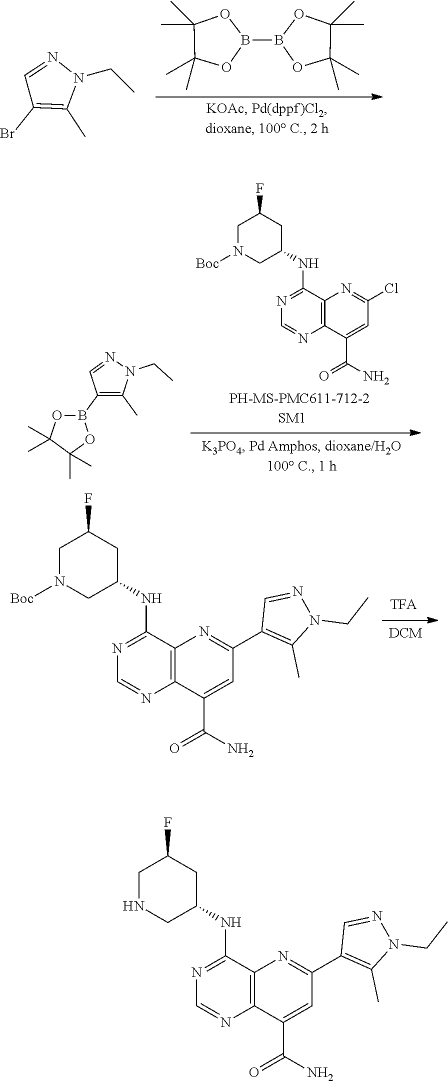

1-ethyl-5-methyl-4-(4,4,5,5-tetramethyl-1,3,2-dioxaborolan-2-yl)-1H-pyrazole: To a stirred mixture of 4-bromo-1-ethyl-5-methyl-1H-pyrazole (200.00 mg; 1.04 mmol) and BPD [Bis(pinacolato)diboron](554.25 mg; 2.07 mmol) in 1,4-Dioxane (15.00 ml) were added Pd(dppf)Cl₂ (79.85 mg; 0.10 mmol) and AcOK (308.32 mg; 3.11 mmol) at room temperature. After stirring for 2 h at 100 degrees C. under argon atmosphere. The mixture was concentrated under reduced pressure. The residue was purified by silica gel column chromatography, eluted with PE/EtOAc (4:1) to afford 1-ethyl-5-methyl-4-(4,4,5,5-tetramethyl-1,3,2-dioxaborolan-2-yl)-1H-pyrazole as brown solid (340.00 mg; 97.22%).

tert-butyl(3S,5S)-3-((8-carbamoyl-6-(1-ethyl-5-methyl-1H-pyrazol-4-yl)pyrido[3,2-d]pyrimidin-4-yl)amino)-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (120.00 mg; 0.22 mmol) and 1-ethyl-5-methyl-4-(4,4,5,5-tetramethyl-1,3,2-dioxaborolan-2-yl)-1H-pyrazole (111.78 mg; 0.33 mmol) in DME (10.00 ml) and H₂O (2.00 ml) were added Pd(PPh₃)₄ (26.87 mg; 0.02 mmol) and Na₂CO₃ (73.95 mg; 0.66 mmol) at room temperature. The resulting mixture was stirred for 2 h at 100° C. under argon atmosphere. The mixture was concentrated under reduced pressure. The residue was purified by silica gel column chromatography, eluted with CH₂Cl₂/MeOH (14:1) to afford tert-butyl (3S,5S)-3-{[8-carbamoyl-6-(1-ethyl-5-methyl-1H-pyrazol-4-yl)pyrido[3,2-d]pyrimidin-4-yl]amino}-5-fluoropiperidine-1-carboxylate as yellow solid (110.00 mg; 85.52%).

6-(1-ethyl-5-methyl-1H-pyrazol-4-yl)-4-(((3S,5S)-5-fluoropiperidin-3-yl)amino)pyrido[3,2-d]pyrimidine-8-carboxamide: To a stirred mixture of tert-butyl (3S,5S)-3-{[8-carbamoyl-6-(1-ethyl-5-methyl-1H-pyrazol-4-yl)pyrido[3,2-d]pyrimidin-4-yl]amino}-5-fluoropiperidine-1-carboxylate (140.00 mg; 0.27 mmol) and TFA (1.00 ml) in DCM (6.00 ml) at room temperature. After stirring for 1 h at room temperature. The mixture was concentrated under reduced pressure. The crude product was purified by Prep-HPLC(Column: XBridge Shield RP18 OBD Column, 30*150 mm, 5 μm; Mobile Phase A: Water (10 mmol/L NH₄HCO₃+0.1% [28% NH₃ in H₂O]), Mobile Phase B: ACN; Flow rate: 60 mL/min; Gradient: 17% B to 44% B in 9 min, 44% B; Wave Length: 254 nm; RT1 (min): 7; Number Of Runs: 2) to afford 6-(1-ethyl-5-methyl-1H-pyrazol-4-yl)-4-{[(3S,5S)-5-fluoropiperidin-3-yl]amino}pyrido[3,2-d]pyrimidine-8-carboxamide as yellow solid (12.40 mg; 11.33%).

HPLC: 98.92% purity, 2.21 min. MS: m/z=399.2 [M+H]⁺. ¹H NMR (400 MHz, DMSO, ppm): b 1.36 (t, J=7.2 Hz, 3H), 2.18 (dt, J=13.4, 4.1 Hz, 2H), 2.68 (s, 3H), 2.72 (ddd, J=12.3, 8.3, 1.9 Hz, 2H), 2.81 (dd, J=13.3, 2.6 Hz, 1H), 3.03 (dd, J=12.3, 3.8 Hz, 1H), 4.20 (q, J=7.2 Hz, 2H), 4.53 (dq, J=9.4, 5.2, 4.8 Hz, 1H), 4.88 (d, J=4.6 Hz, 1H), 7.86 (d, J=8.7 Hz, 1H), 8.20 (d, J=7.9 Hz, 2H), 8.54 (d, J=11.8 Hz, 2H), 10.01 (d, J=3.7 Hz, 1H).

### Example 9: Synthesis of Compound 9 6-(1-ethyl-3-methyl-1H-pyrazol-4-yl)-4-(((3S,5S)-5-fluoropiperidin-3-yl)amino)pyrido[3,2-d]pyrimidine-8-carboxamide

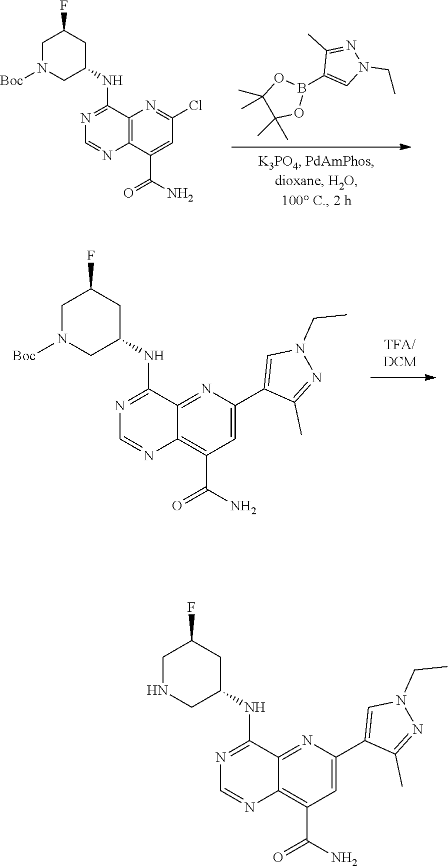 [SMILES: CC(C)(C)OC(=O)N1C[C@@H](F)C[C@H](Nc2ncnc3c(C(N)=O)cc(Cl)nc23)C1 ; CCn1cc(B2OC(C)(C)C(C)(C)O2)c(C)n1 ; CCn1cc(-c2cc(C(N)=O)c3ncnc(N[C@H]4C[C@H](F)CN(C(=O)OC(C)(C)C)C4)c3n2)c(C)n1 ; CCn1cc(-c2cc(C(N)=O)c3ncnc(N[C@@H]4CNC[C@@H](F)C4)c3n2)c(C)n1]

tert-butyl(3S,5S)-3-((8-carbamoyl-6-(1-ethyl-3-methyl-1H-pyrazol-4-yl)pyrido[3,2-d]pyrimidin-4-yl)amino)-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (120.00 mg; 0.22 mmol) and 1-ethyl-3-methyl-4-(4,4,5,5-tetramethyl-1,3,2-dioxaborolan-2-yl)-1H-pyrazole (111.78 mg; 0.33 mmol) in DME (10.00 ml) and H₂O (2.00 ml) were added P₄ (26.87 mg; 0.02 mmol) and Na₂CO₃ (73.95 mg; 0.66 mmol) at room temperature. The resulting mixture was stirred for 2 h at 100° C. under argon atmosphere. The mixture was concentrated under reduced pressure. The residue was purified by silica gel column chromatography, eluted with CH₂Cl₂/MeCOH (14:1) to afford tert-butyl (3S,5S)-3-{[8-carbamoyl-6-(1-ethyl-5-methyl-1H-pyrazol-4-yl)pyrido[3,2-d]pyrimidin-4-yl]amino}-5-fluoropiperidine-1-carboxylate as yellow solid (100 mg; 71.42%).

6-(1-ethyl-3-methyl-1H-pyrazol-4-yl)-4-(((3S,5S)-5-fluoropiperidin-3-yl)amino)pyrido[3,2-d]pyrimidine-8-carboxamide: To a stirred mixture of tert-butyl (3S,5S)-3-{[8-carbamoyl-6-(1-ethyl-3-methyl-1H-pyrazol-4-yl)pyrido[3,2-d]pyrimidin-4-yl]amino}-5-fluoropiperidine-1-carboxylate (100.00 mg; 0.19 mmol) in DCM (6.00 ml) and TFA (1.00 ml) at room temperature. The resulting mixture was stirred for 1 h at room temperature. The mixture was concentrated under reduced pressure. The crude product was purified by Prep-HPLC(Column: XBridge Shield RP18 OBD Column, 30*150 mm, 5 μm; Mobile Phase A: Water (10 mmol/L NH₄HCO₃+0.1% [28% NH₃ in H₂O]), Mobile Phase B: ACN; Flow rate: 60 mL/min.; Gradient: 17% B to 44% B in 9 min., 44% B; Wave Length: 254 nm; RT1 (min.): 7) to afford 6-(1-ethyl-3-methyl-1H-pyrazol-4-yl)-4-{[(3S,5S)-5-fluoropiperidin-3-yl]amino}pyrido[3,2-d]pyrimidine-8-carboxamide as yellow solid (24.20 mg; 30.76%).

HPLC: 97.51% purity, RT=4.97 min. MS: m/z=399.2[M+H]⁺. ¹H NMR (300 MHz, DMSO-d6, ppm): δ 9.91 (d, J=3.8 Hz, 1H), 8.49 (d, J=2.3 Hz, 1H), 8.42 (s, 2H), 8.08 (d, J=3.8 Hz, 1H), 7.66 (d, J=8.6 Hz, 1H), 4.87-4.53 (m, 1H), 4.37 (s, 1H), 4.01 (q, J=7.3 Hz, 2H), 3.01-2.53 (m, 4H), 2.43 (s, 3H), 2.05 (s, 1H), 1.99 (s, 1H), 1.30 (t, J=7.3 Hz, 3H).

### Example 10: Synthesis of Compound 10 (S)-6-(2-cyano-4-(difluoromethyl)phenyl)-4-((5,5-difluoropiperidin-3-yl)amino)pyrido[3,2-d]pyrimidine-8-carboxamide

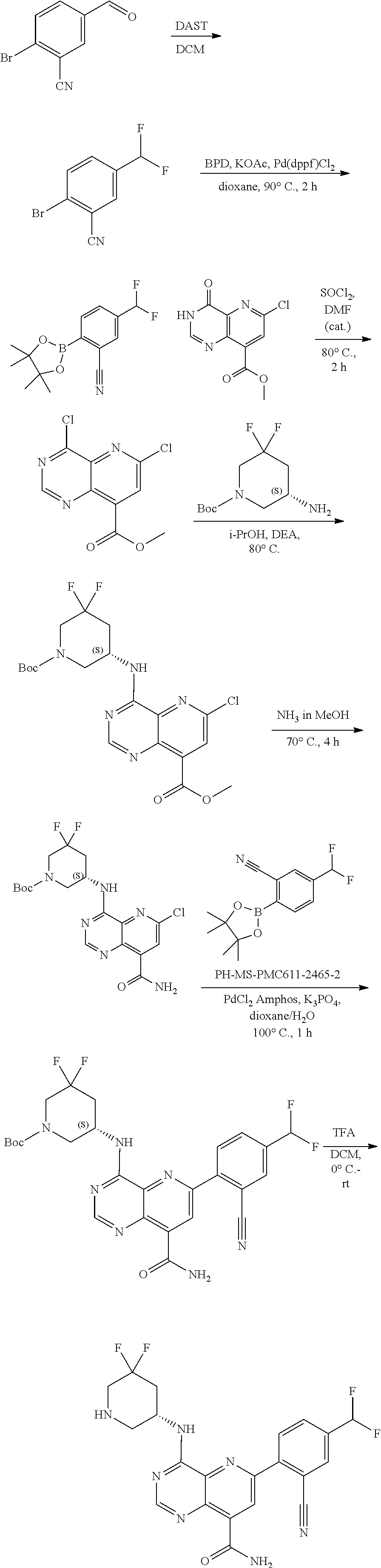

2-bromo-5-(difluoromethyl)benzonitrile: To a stirred mixture of 2-bromo-5-formylbenzonitrile (1.00 g; 4.52 mmol) in DCM (40.00 ml) was added DAST (1.19 ml; 11.08 mmol) dropwise at 0° C. The resulting mixture was stirred for 2 h at room temperature under nitrogen atmosphere. The reaction was quenched with saturated NaHCO₃ at 0 degrees C. and extracted with DCM (3×50 mL). The combined organic layers were washed with brine, dried over anhydrous Na₂SO₄. After filtration, the filtrate was concentrated under reduced pressure to afford 2-bromo-5-(difluoromethyl)benzonitrile as a light-yellow solid (1.11 g; Crude).

5-(difluoromethyl)-2-(4,4,5,5-tetramethyl-1,3,2-dioxaborolan-2-yl)benzonitrile: To a stirred mixture of 2-bromo-5-(difluoromethyl)benzonitrile (180.00 mg; 0.73 mmol) and BPD (582.29 mg; 2.18 mmol) in 1,4-Dioxane (5.00 ml) were added KOAc (237.54 mg; 2.18 mmol) and Pd(dppf)Cl₂ (55.93 mg; 0.07 mmol) at room temperature. After stirring for 2 h at 100° C. under argon atmosphere. The resulting mixture was filtered; the filter cake was washed with dioxane. After filtration, the filtrate was concentrated under reduced pressure. This resulted in 5-(difluoromethyl)-2-(4,4,5,5-tetramethyl-1,3,2-dioxaborolan-2-yl)benzonitrile as brown solid (240 mg; Crude).

methyl 4,6-dichloropyrido[3,2-d]pyrimidine-8-carboxylate: To a stirred mixture of methyl 6-chloro-4-hydroxypyrido[3,2-d]pyrimidine-8-carboxylate (2.18 g; 8.07 mmol) in SOCl₂ (20.00 ml; 264.07 mmol) at room temperature. The resulting mixture was stirred for 4 h at 80° C. The filtrate was concentrated under reduced pressure. This resulted in methyl 4,6-dichloropyrido[3,2-d]pyrimidine-8-carboxylate as yellow solid (2.30 g; Crude) used in the next step without purification.

methyl(S)-4-((1-(tert-butoxycarbonyl)-5,5-difluoropiperidin-3-yl)amino)-6-chloropyrido[3,2-d]pyrimidine-8-carboxylate: To a stirred mixture of methyl 4,6-dichloropyrido[3,2-d]pyrimidine-8-carboxylate (700.00 mg; 1.81 mmol) and tert-butyl (5S)-5-amino-3,3-difluoropiperidine-1-carboxylate (676.95 mg; 2.72 mmol) in ACN (15.00 ml) was added DIEA (987.19 mg; 7.26 mmol) at room temperature. The resulting mixture was stirred for 2 h at 75° C. The resulting mixture was concentrated under reduced pressure and purified by silica gel column chromatography, eluted with PE/EtOAc (1:19) to afford tert-butyl (5S)-5-{[6-chloro-8-(methoxycarbonyl)pyrido[3,2-d]pyrimidin-4-yl]amino}-3,3-difluoropiperidine-1-carboxylate as yellow solid (380 mg 40.67%).

tert-butyl(S)-5-((8-carbamoyl-6-chloropyrido[3,2-d]pyrimidin-4-yl)amino)-3,3-difluoropiperidine-1-carboxylate: To a stirred mixture of tert-butyl (5S)-5-{[6-chloro-8-(methoxycarbonyl)pyrido[3,2-d]pyrimidin-4-yl]amino}-3,3-difluoropiperidine-1-carboxylate (380.00 mg; 0.74 mmol; 1.00 eq.) in NH₃ (g) in MeOH (5.00 ml) at room temperature. After stirring for 2 h at 40 degrees C. The resulting mixture was concentrated under reduced pressure and purified by silica gel column chromatography, eluted with PE/EtOAc (1:6) to afford tert-butyl (5S)-5-({8-carbamoyl-6-chloropyrido[3,2-d]pyrimidin-4-yl}amino)-3,3-difluoropiperidine-1-carboxylate (320.00 mg; 0.64 mmol; 86.76%; yellow solid; Purified Product).

tert-butyl (S)-5-((8-carbamoyl-6-(2-cyano-4-(difluoromethyl)phenyl)pyrido[3,2-d]pyrimidin-4-yl)amino)-3,3-difluoropiperidine-1-carboxylate: To a stirred mixture of tert-butyl (5S)-5-({8-carbamoyl-6-chloropyrido[3,2-d]pyrimidin-4-yl}amino)-3,3-difluoropiperidine-1-carboxylate (160.00 mg; 0.32 mmol) and 5-(difluoromethyl)-2-(4,4,5,5-tetramethyl-1,3,2-dioxaborolan-2-yl)benzonitrile (223.10 mg; 0.48 mmol) in 1,4-Dioxane (5.00 ml) and water (1.00 ml) were added Pd(dtbpf)Cl₂ (21.94 mg; 0.03 mmol) and K₃PO₄ (214.33 mg; 0.96 mmol) at room temperature. After stirring for 2 h at 80° C. under argon atmosphere. The resulting mixture was concentrated under reduced pressure and purified by silica gel column chromatography, eluted with EtOAc/MeOH (13:1) to afford tert-butyl (5S)-5-({8-carbamoyl-6-[2-cyano-4-(difluoromethyl)phenyl]pyrido[3,2-d]pyrimidin-4-yl}amino)-3,3-difluoropiperidine-1-carboxylate as brown solid (190 mg, 93.88%).

(S)-6-(2-cyano-4-(difluoromethyl)phenyl)-4-((5,5-difluoropiperidin-3-yl)amino)pyrido[3,2-d]pyrimidine-8-carboxamide: To a solution of tert-butyl (5S)-5-({8-carbamoyl-6-[2-cyano-4-(difluoromethyl)phenyl]pyrido[3,2-d]pyrimidin-4-yl}amino)-3,3-difluoropiperidine-1-carboxylate (180 mg; 0.28 mmol) in DCM (9.00 ml) were added TFA (3.00 ml) at room temperature. The resulting mixture was stirred for 1 h at room temperature. The resulting mixture was concentrated under reduced pressure and purified by Prep-HPLC(Column: YMC-Actus Triart C18 ExRS, 30*150 mm, 5 μm; Mobile Phase A: Water (10 mmol/L NH₄HCO₃+0.1% [28% NH₃ in H₂O]), Mobile Phase B: ACN; Flow rate: 60 mL/min.; Gradient: 25% B to 55% B in 8 min., 55% B; Wave Length: 254 nm; RT1 (min.): 7; Number Of Runs: 2) to afford 6-[2-cyano-4-(difluoromethyl)phenyl]-4-{[(3S)-5,5-difluoropiperidin-3-yl]amino}pyrido[3,2-d]pyrimidine-8-carboxamide as yellow solid (49.40 mg; 37.61%).

HPLC 99.48% purity, RT=3.62 min. MS: m/z=460.15 [M+H]⁺. ¹H NMR (400 MHz, DMSO-d6, ppm): δ 9.91 (s, 1H), 8.91 (s, 1H), 8.71 (s, 1H), 8.46 (d, J=8.2 Hz, 1H), 8.33 (d, J=13.3 Hz, 2H), 8.22 (d, J=8.5 Hz, 1H), 8.11 (d, J=8.3 Hz, 1H), 7.23 (s, 1H), 4.53 (d, J=10.4 Hz, 1H), 3.07 (dd, J=24.6, 11.5 Hz, 2H), 2.82 (s, 2H), 2.65 (s, 1H), 2.28-2.15 (m, 1H).

### Example 11: Synthesis of Compound 11 (S)-4-((5,5-difluoropiperidin-3-yl)amino)-6-(4-methoxyphenyl)pyrido[3,2-d]pyrimidine-8-carboxamide

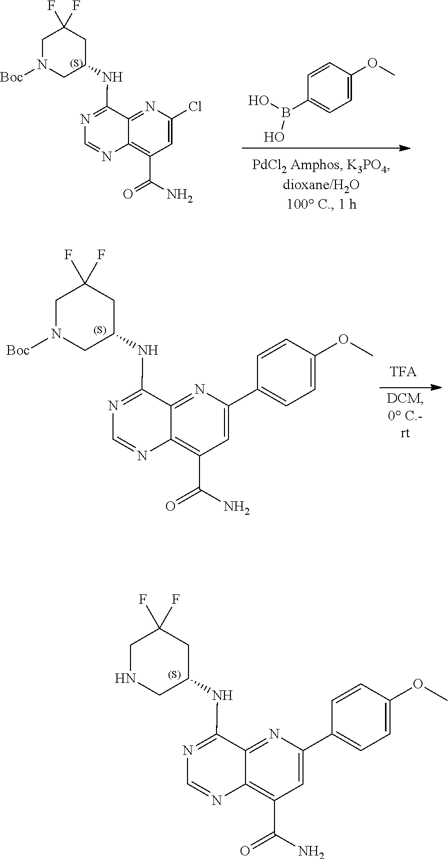

tert-butyl (S)-5-((8-carbamoyl-6-(4-methoxyphenyl)pyrido[3,2-d]pyrimidin-4-yl)amino)-3,3-difluoropiperidine-1-carboxylate: To a stirred mixture of tert-butyl (5S)-5-({8-carbamoyl-6-chloropyrido[3,2-d]pyrimidin-4-yl}amino)-3,3-difluoropiperidine-1-carboxylate (120.00 mg; 0.21 mmol) and (4-methoxyphenyl)boronic acid (40.99 mg; 0.26 mmol) in 1,4-Dioxane (0.50 ml) and water (0.10 ml) were added Pd(dtbpf)Cl₂ (14.65 mg; 0.02 mmol) and K₃PO₄ (143.13 mg; 0.64 mmol) at room temperature. The resulting mixture was stirred for 2 h at 80° C. under argon atmosphere. The solvent was removed under vacuum and the residue was purified by silica gel column chromatography, eluted with CH₂Cl₂/MeOH (12:1) to afford tert-butyl (5S)-5-{[8-carbamoyl-6-(4-methoxyphenyl)pyrido[3,2-d]pyrimidin-4-yl]amino}-3,3-difluoropiperidine-1-carboxylate as a yellow solid (100 mg; 77.6%).

(S)-4-((5,5-difluoropiperidin-3-yl)amino)-6-(4-methoxyphenyl)pyrido[3,2-d]pyrimidine-8-carboxamide: To a solution of tert-butyl (5S)-5-{[8-carbamoyl-6-(4-methoxyphenyl)-1,5-naphthyridin-4-yl]amino}-3,3-difluoropiperidine-1-carboxylate (100 mg; 0.18 mmol) in DCM (2 ml) were added TFA (1 ml). The resulting mixture was stirred for 12 h at 25° C. The resulting mixture was concentrated under reduced pressure. The residue was purified by Prep-HPLC to afford 8-{[(3S)-5,5-difluoropiperidin-3-yl]amino}-2-(4-methoxyphenyl)-1,5-naphthyridine-4-carboxamide as a yellow solid (55.20 mg, 74.7%).

HPLC 99.16% purity, RT=3.26 min. MS:m/z=415.05 [M+H]⁺. ¹H NMR (300 MHz, DMSO-d6, ppm): δ 10.01 (d, 1H), 8.82 (s, 1H), 8.59 (s, 1H), 8.44 (d, 1H), 8.40-8.31 (m, 2H), 8.25 (d, 1H), 7.18-7.07 (m, 2H), 4.60 (s, 1H), 3.87 (s, 3H), 3.34 (s, 2H), 3.06 (d, 3H), 2.49-2.35 (m, 1H).

### Example 12: Synthesis of Compound 12 (4-(((3S,5S)-5-fluoropiperidin-3-yl)amino)-6-(4-(2-hydroxy-2-methylpropoxy)phenyl)pyrido[3,2-d]pyrimidine-8-carboxamide)

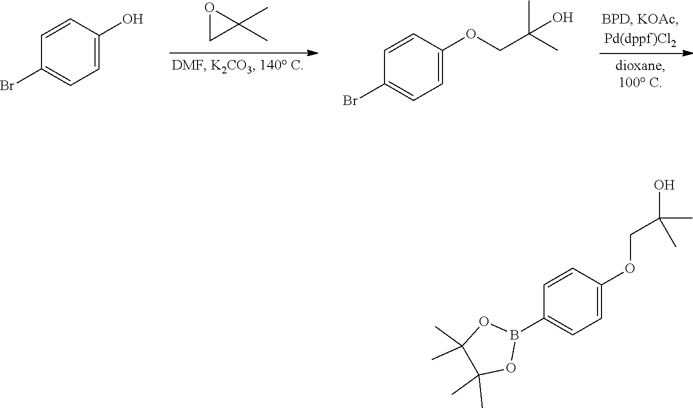

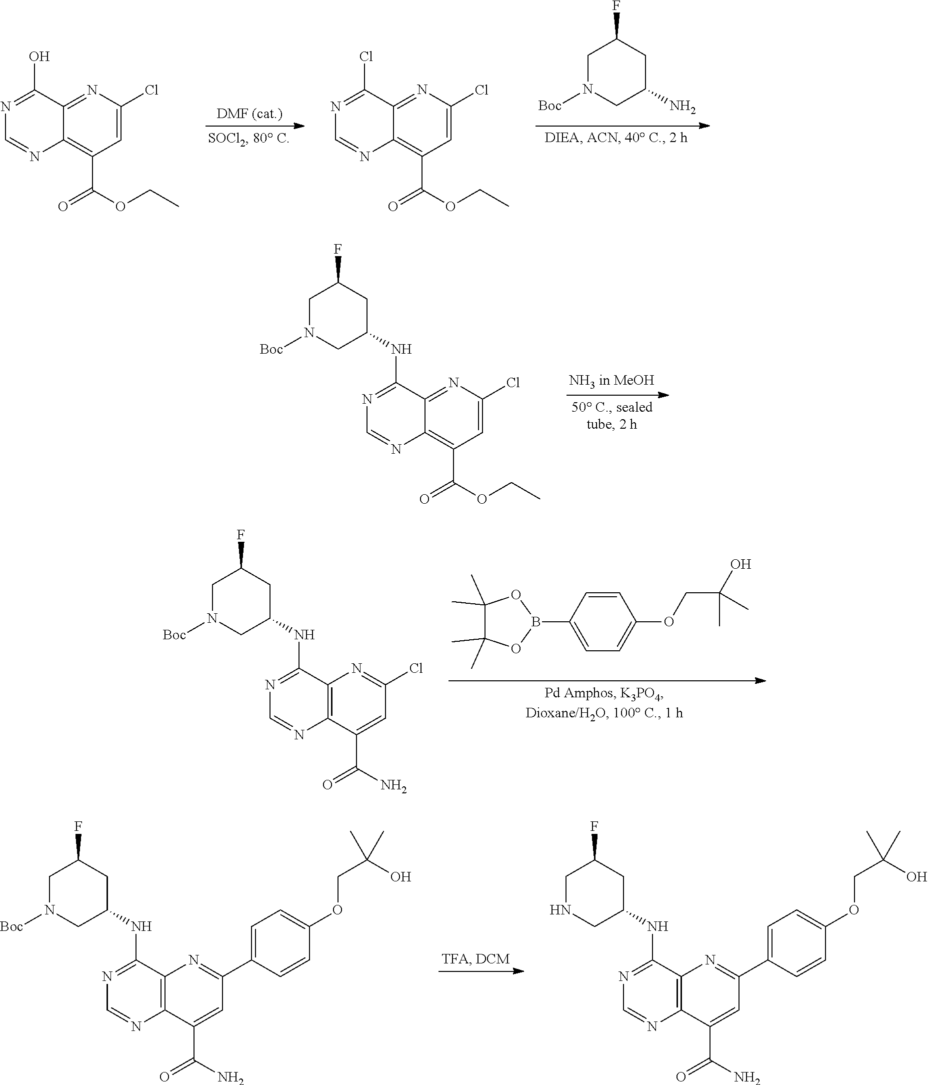

1-(4-bromophenoxy)-2-methylpropan-2-ol: To a solution of 4-bromophenol (20.00 g; 109.82 mmol; 1.00 eq.) and 2,2-dimethyloxirane (12.50 g; 164.73 mmol) in DMF (100.00 ml) was added K₂CO₃ (47.93 g; 329.47 mmol) in portions at room temperature under nitrogen atmosphere. The resulting mixture was stirred for overnight at 140° C. under nitrogen atmosphere. The resulting mixture was diluted with water (50 mL), extracted with EtOAc (3×50 mL). The combined organic layers were washed with brine (50 mL), dried over anhydrous Na₂SO₄. The resulting mixture was concentrated under reduced pressure to afford 1-(4-bromophenoxy)-2-methylpropan-2-ol (32.00 g, crude product) as a yellow oil.

2-methyl-1-[4-(4,4,5,5-tetramethyl-1,3,2-dioxaborolan-2-yl)phenoxy]propan-2-ol: To a stirred mixture of 1-(4-bromophenoxy)-2-methylpropan-2-ol (30.00 g; 122.39 mmol) and BPD (32.72 g; 122.39 mmol) in 1,4-Dioxane (600.00 ml) was added KOAc (37.93 g; 367.18 mmol; 3.00 eq.) and Pd(dppf)Cl₂ (4.71 g; 6.12 mmol; 0.05 eq.) in portions at room temperature. The resulting mixture was stirred for 1 h at 100° C. under nitrogen atmosphere. The resulting mixture was diluted with water (500 mL), extracted with EtOAc (3×500 mL). The combined organic layers were washed with brine (500 mL), dried over anhydrous Na₂SO₄. The resulting mixture was concentrated under reduced pressure. The residue was purified by silica gel column eluted with EtOAc:PE=1:6 to afford 2-methyl-1-[4-(4,4,5,5-tetramethyl-1,3,2-dioxaborolan-2-yl)phenoxy]propan-2-ol (30.00 g; 83.89%) as a white solid.

ethyl 4,6-dichloropyrido[3,2-d]pyrimidine-8-carboxylate: To a stirred mixture of methyl 6-chloro-4-oxo-3H,4H-pyrido[3,2-d]pyrimidine-8-carboxylate (500.00 mg; 1.71 mmol) in SOCl₂ (5.00 ml; 66.02 mmol) was added DMF (3 drops) at room temperature. The resulting mixture was stirred for 4 h (at this time the mixture is a clear solution) at 80° C. under nitrogen atmosphere. The resulting mixture was concentrated under reduced pressure and co-evaporated with DCM 4 times to afford methyl 4,6-dichloropyrido[3,2-d]pyrimidine-8-carboxylate (520.00 mg; crude product) as a brown solid. The residue was used for next step directly without further purification.

ethyl 4-(((3S,5S)-1-(tert-butoxycarbonyl)-5-fluoropiperidin-3-yl)amino)-6-chloropyrido[3,2-d]pyrimidine-8-carboxylate: To a stirred mixture of methyl 4,6-dichloropyrido[3,2-d]pyrimidine-8-carboxylate (500.00 mg; 1.55 mmol) and tert-butyl (3S,5S)-3-amino-5-fluoropiperidine-1-carboxylate (466.00 mg; 2.03 mmol) in MeCN (7.00 ml) was added DIEA (1.01 ml; 5.53 mmol) dropwise at room temperature. The resulting mixture was stirred for 1 h at 40° C. under nitrogen atmosphere. The resulting mixture was added 50 ml water and extracted with EA (3×30 mL). he combined organic layers were washed with brine, dried over anhydrous Na₂SO₄. After filtration, the filtrate was concentrated under reduced pressure to afford tert-butyl (3S,5S)-3-{[6-chloro-8-(methoxycarbonyl) pyrido[3,2-d]pyrimidin-4-yl]amino}-5-fluoropiperidine-1-carboxylate (800.00 mg; crude product) as brown solid.

tert-butyl (3S,5S)-3-((8-carbamoyl-6-chloropyrido[3,2-d]pyrimidin-4-yl)amino)-5-fluoropiperidine-1-carboxylate: A stirred mixture of tert-butyl (3S,5S)-3-{[6-chloro-8-(methoxycarbonyl) pyrido[3,2-d]pyrimidin-4-yl]amino}-5-fluoropiperidine-1-carboxylate (750.00 mg; 1.45 mmol) in NH₃ in MeOH (10.00 ml) was stirred for 1 h at 40° C. The resulting mixture was concentrated under reduced pressure to afford tert-butyl (3S,5S)-3-({8-carbamoyl-6-chloropyrido[3,2-d]pyrimidin-4-yl}amino)-5-fluoropiperidine-1-carboxylate (740.00 mg; crude product) as a brown solid.

tert-butyl (3S,5S)-3-((8-carbamoyl-6-(4-(2-hydroxy-2-methylpropoxy)phenyl)pyrido[3,2-d]pyrimidin-4-yl)amino)-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (200.00 mg; 0.39 mmol) and 2-methyl-1-[4-(4,4,5,5-tetramethyl-1,3,2-dioxaborolan-2-yl)phenoxy]propan-2-ol (290.00 mg; 0.94 mmol) in 1,4-dioxane (5.00 ml) and H₂O (0.50 ml) was added K₃PO₄ (200.00 mg; 0.90 mmol) and AMPHOS-PdCl₂ (34.00 mg; 0.05 mmol) in portions at room temperature. The resulting mixture was stirred for 2 h at 100° C. under nitrogen atmosphere. The resulting mixture was concentrated under reduced pressure. The residue was purified by silica gel column chromatography, eluted with 63% EA in PE to afford tert-butyl (3S,5S)-3-({8-carbamoyl-6-[4-(2-hydroxy-2-methylpropoxy)phenyl]pyrido[3,2-d]pyrimidin-4-yl}amino)-5-fluoropiperidine-1-carboxylate (180.00 mg; 63.0%) as a yellow solid.

4-(((3S,5S)-5-fluoropiperidin-3-yl)amino)-6-(4-(2-hydroxy-2-methylpropoxy)phenyl)pyrido[3,2-d]pyrimidine-8-carboxamide: To a stirred mixture of tert-butyl (3S,5S)-3-({8-carbamoyl-6-[4-(2-hydroxy-2-methylpropoxy)phenyl]pyrido[3,2-d]pyrimidin-4-yl}amino)-5-fluoropiperidine-1-carboxylate (160.00 mg; 0.22 mmol; 1.00 eq.) in DCM (3.00 ml) was added TFA (1.00 ml) dropwise at 0° C. The resulting mixture was stirred for 1 h at room temperature under nitrogen atmosphere. The resulting mixture was concentrated under reduced pressure. The crude product was purified by Prep-HPLC with the following conditions (Column: XBridge Prep OBD C18 Column, 30*150 mm; Mobile Phase A: Water (10 mmol/L NH₄HCO₃+0.1% NH₃·H₂O), Mobile Phase B: ACN; Flow rate: 60 mL/min.; Gradient: 20% B to 45% B in 9 min., 45% B; Wave Length: 254 nm; RT1 (min.): 7; Number Of Runs: 4) to afford 4-{[(3S,5S)-5-fluoropiperidin-3-yl]amino}-6-[4-(2-hydroxy-2-methylpropoxy)phenyl]pyrido[3,2-d]pyrimidine-8-carboxamide (71.00 mg; 70.3%) as a yellow solid.

HPLC: 98.3% purity, RT=2.94 min. MS: m/z=455.05 [M+H]⁺. ¹H NMR (400 MHz, DMSO-d6) δ 10.06 (d, J=3.7 Hz, 1H), 8.81 (s, 1H), 8.56 (s, 1H), 8.43-8.30 (m, 3H), 8.22 (d, J=3.8 Hz, 1H), 7.11 (d, J=8.4 Hz, 2H), 4.87 (d, J=48.1 Hz, 1H), 4.70 (s, 1H), 4.60 (d, J=10.4 Hz, 1H), 3.83 (s, 2H), 3.34 (s, 1H), 3.13-2.92 (m, 2H), 2.74 (td, J=20.4, 19.4, 10.9 Hz, 2H), 2.35-2.12 (m, 2H), 1.24 (s, 6H).

### Example 13: Synthesis of Compound 13 (S)-6-(3-(methoxymethyl)-1H-1,2,4-triazol-5-yl)-4-(piperidin-3-ylamino)pyrido[3,2-d]pyrimidine-8-carboxamide

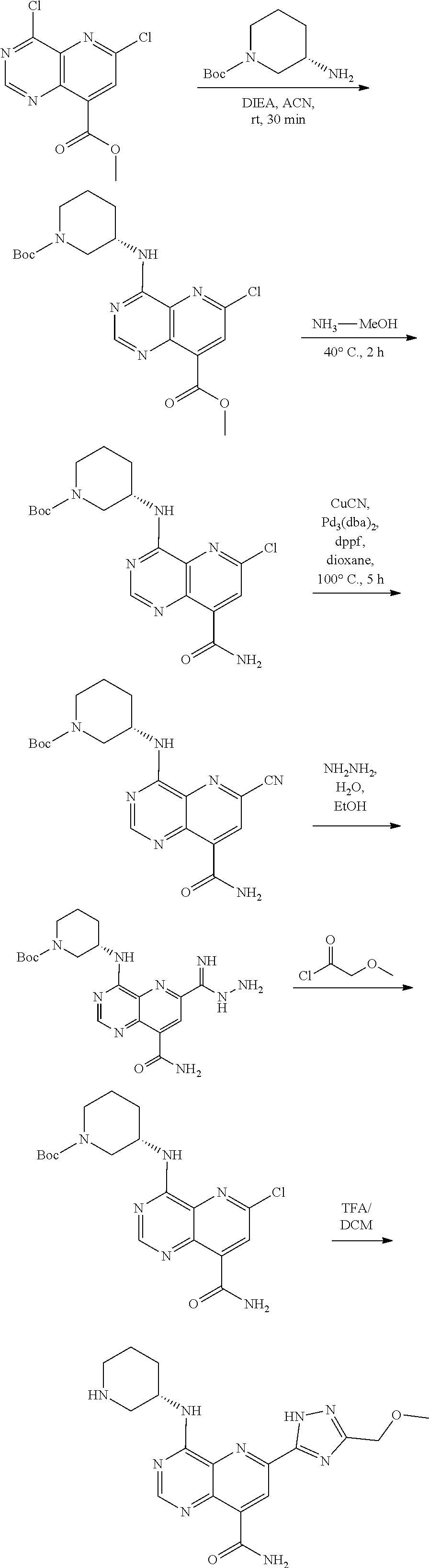

methyl (S)-4-((1-(tert-butoxycarbonyl)piperidin-3-yl)amino)-6-chloropyrido[3,2-d]pyrimidine-8-carboxylate: To a mixture of methyl 4,6-dichloropyrido[3,2-d]pyrimidine-8-carboxylate (3.10 g; 9.87 mmol) and tert-butyl (3S)-3-aminopiperidine-1-carboxylate (2.29 g; 10.86 mmol) in ACN (30.00 ml) was added ethylbis(propan-2-yl)amine (4.03 g; 29.61 mmol). After stirring for 2 h at 40 degrees C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (70:30) to afford tert-butyl (3S)-3-{[6-chloro-8-(methoxycarbonyl)pyrido[3,2-d]pyrimidin-4-yl]amino}piperidine-1-carboxylate (2.80 g, 64.6%) as a yellow solid.

tert-butyl(S)-3-((8-carbamoyl-6-chloropyrido[3,2-d]pyrimidin-4-yl)amino)piperidine-1-carboxylate: A mixture of tert-butyl (3S)-3-{[6-chloro-8-(methoxycarbonyl)pyrido[3,2-d]pyrimidin-4-yl]amino}piperidine-1-carboxylate (2.80 g; 6.37 mmol) in NH₃ (g) in MeOH (20.00 ml) was stirred for 2 h at 40° C. under nitrogen atmosphere. The resulting mixture was concentrated under reduced pressure. This resulted in tert-butyl (3S)-3-({8-carbamoyl-6-chloropyrido[3,2-d]pyrimidin-4-yl}amino)piperidine-1-carboxylate (2.30 g, Crude) as a light-yellow solid used in the next step without purification.

tert-butyl(S)-3-((8-carbamoyl-6-cyanopyrido[3,2-d]pyrimidin-4-yl)amino)piperidine-1-carboxylate: To a stirred mixture of tert-butyl (S)-3-((8-carbamoyl-6-chloropyrido[3,2-d]pyrimidin-4-yl)amino)piperidine-1-carboxylate (800 mg; 1.97 mmol) in 1,4-Dioxane (15 ml) were added CuCN (355 mg; 3.94 mmol), Tris(dibenzylideneacetone)dipalladium (180 mg; 0.197 mmol) and dppf (218 mg; 0.39 mmol) at room temperature. The resulting mixture was stirred for 5 h at 100° C. under argon atmosphere. The mixture was concentrated under reduced pressure. The residue was purified by silica gel column chromatography, eluted with PE/EtOAc (1:8) to afford tert-butyl (S)-3-((8-carbamoyl-6-cyanopyrido[3,2-d]pyrimidin-4-yl)amino)piperidine-1-carboxylate as yellow solid (397 mg, 50.76%).

tert-butyl(S)-3-((8-carbamoyl-6(hydrazineyl(imino)methyl)pyrido[3,2-d]pyrimidin-4-yl)amino)piperidine-1-carboxylate: A mixture of tert-butyl (3S)-3-({8-carbamoyl-6-cyanopyrido[3,2-d]pyrimidin-4-yl}amino)piperidine-1-carboxylate (320.00 mg; 0.70 mmol) and diazene hydrate hydrogen (0.17 ml; 2.81 mmol) in EtOH (20.00 ml) was stirred for 16 h at 80 degrees C. The resulting mixture was concentrated under reduced pressure. The residue was purified by silica gel column chromatography, eluted with CH₂Cl₂/MeOH (9:1) to afford tert-butyl (3S)-3-{[6-(N-aminocarbamimidoyl)-8-carbamoylpyrido[3,2-d]pyrimidin-4-yl]amino}piperidine-1-carboxylate as yellow solid (230.00 mg, 68.7%).

methyl(S)-4-((1-(tert-butoxycarbonyl)piperidin-3-yl)amino)-6-chloropyrido[3,2-d]pyrimidine-8-carboxylate: To a stirred solution of tert-butyl (3S)-3-{[6-(N-aminocarbamimidoyl)-8-carbamoylpyrido[3,2-d]pyrimidin-4-yl]amino}piperidine-1-carboxylate (230.00 mg; 0.49 mmol) in DMA (10.00 ml) and THF (2.00 ml) was added Na₂CO₃ (54.44 mg; 0.49 mmol). The mixture was allowed to cool down to 0 degrees C. Then the above mixture was added 2-methoxyacetyl chloride (55.73 mg; 0.49 mmol) in DMA (3.00 ml) dropwise. The resulting mixture was stirred for additional 1 h at 50 degrees C. The reaction was quenched with Water/Ice. The organic layer was extracted with EtOAc (3×100 mL), washed with 1×100 mL of brine, dried over Na₂SO₄. The resulting mixture was concentrated under reduced pressure. The resulting mixture was added phenoxybenzene (20.00 ml). After the resulting mixture was stirred for additional 2 h at 180° C. The residue was purified by silica gel column chromatography, eluted with CH₂Cl₂/MeOH (9:1) to afford tert-butyl (3S)-3-({8-carbamoyl-6-[3-(methoxymethyl)-1H-1,2,4-triazol-5-yl]pyrido[3,2-d]pyrimidin-4-yl}amino)piperidine-1-carboxylate as yellow solid (150.00 mg; 39.3%).

tert-butyl (S)-3-((8-carbamoyl-6-(3-(methoxymethyl)-1H-1,2,4-triazol-5-yl)pyrido[3,2-d]pyrimidin-4-yl)amino)piperidine-1-carboxylate: To a stirred mixture of tert-butyl (3S)-3-({8-carbamoyl-6-[3-(methoxymethyl)-1H-1,2,4-triazol-5-yl]pyrido[3,2-d]pyrimidin-4-yl}amino)piperidine-1-carboxylate (120.00 mg; 0.15 mmol) and TFA (2 ml) in DCM (5 ml) at room temperature. The resulting mixture was stirred for 1 h at room temperature. The resulting mixture was concentrated under reduced pressure. The residue was purified by Prep-HPLC(Column: XBridge Prep OBD C18 Column, 30*150 mm, 5 μm; Mobile Phase A: Water (10 mmoL/L NH₄HCO₃+0.1% [28% NH₃ in H₂O]), Mobile Phase B: ACN; Flow rate: 60 mL/min.; Gradient: 10% B to 40% B in 8 min., 40% B; Wave Length: 254 nm; RT1 (min.): 6; Number Of Runs: 1) to afford 6-[3-(methoxymethyl)-1H-1,2,4-triazol-5-yl]-4-{[(3S)-piperidin-3-yl]amino}pyrido[3,2-d]pyrimidine-8-carboxamide as light yellow solid (6.60 mg, 11.0%).

HPLC 98.09% purity, RT=2.88 min. MS: m/z=348.10 [M+H]⁺. ¹H NMR (400 MHz, Methanol-d4, ppm): 5 9.14-9.07 (m, 1H), 8.55-8.48 (m, 1H), 4.71 (d, 2H), 4.33 (s, 1H), 3.51 (s, 3H), 3.45-3.35 (m, 1H), 3.09 (dt, 1H), 2.80 (td, 2H), 2.22 (d, 1H), 1.99-1.91 (m, 1H), 1.86-1.68 (m, 2H).

### Example 14: Synthesis of Compound 14 6-(4-(((S)-3-cyanomorpholino)methyl)phenyl)-4-(((S)-piperidin-3-yl)amino)pyrido[3,2-d]pyrimidine-8-carboxamide and 6-(4-(((R)-3-cyanomorpholino)methyl)phenyl)-4-(((S)-piperidin-3-yl)amino)pyrido[3,2-d]pyrimidine-8-carboxamide

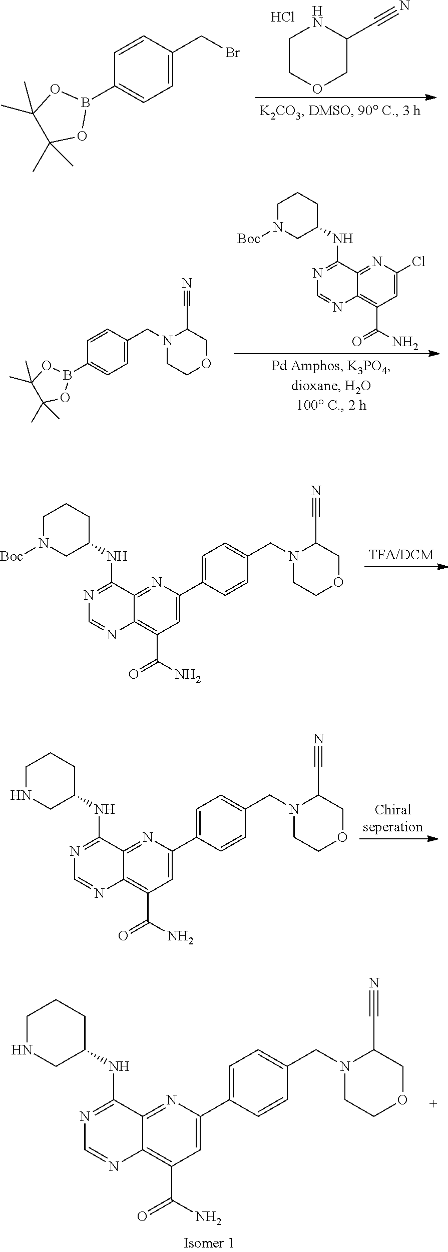

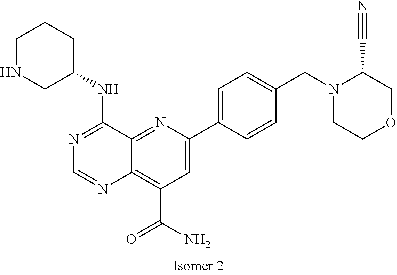 [SMILES: N#C[C@@H]1COCCN1Cc1ccc(-c2cc(C(N)=O)c3ncnc(N[C@H]4CCCNC4)c3n2)cc1]

4-(4-(4,4,5,5-tetramethyl-1,3,2-dioxaborolan-2-yl)benzyl)morpholine-3-carbonitrile: To a stirred mixture of morpholine-3-carbonitrile hydrochloride (0.50 g; 4.24 mmol) and 2-[4-(bromomethyl)phenyl]-4,4,5,5-tetramethyl-1,3,2-dioxaborolane (1.99 g; 6.35 mmol) in CH₃CN (20.00 ml) was added K₂CO₃ (1.23 g; 8.47 mmol) at room temperature. The resulting mixture was stirred for 2 h at 50° C. The residue was directly purified by silica gel column chromatography, eluted with CH₂Cl₂/MeOH (9:1). This resulted in 4-{[4-(4,4,5,5-tetramethyl-1,3,2-dioxaborolan-2-yl)phenyl]methyl}morpholine-3-carbonitrile as light brown solid (1.30 g; 70.03%).

tert-butyl (3S)-3-((8-carbamoyl-6-(4-((3-cyanomorpholino)methyl)phenyl)pyrido[3,2-d]pyrimidin-4-yl)amino)piperidine-1-carboxylate: To a stirred mixture of tert-butyl (3S)-3-({8-carbamoyl-6-chloropyrido[3,2-d]pyrimidin-4-yl}amino)piperidine-1-carboxylate (100.00 mg; 0.23 mmol) and 4-{[4-(4,4,5,5-tetramethyl-1,3,2-dioxaborolan-2-yl)phenyl]methyl}morpholine-3-carbonitrile (152.67 mg; 0.35 mmol) in 1,4-Dioxane (5 ml) and water (1 ml) were added Bis(di-tert-butyl(4-dimethylaminophenyl)phosphine)dichloropalladium(II) (17.32 mg; 0.02 mmol) and K₃PO₄ (155.68 mg; 0.70 mmol) at room temperature. The resulting mixture was stirred for 2 h at 100° C. under argon atmosphere. The residue was purified by silica gel column chromatography, eluted with CH₂Cl₂/MeOH (9:1) to afford tert-butyl (3S)-3-[(8-carbamoyl-6-{4-[(3-cyanomorpholin-4-yl)methyl]phenyl}pyrido[3,2-d]pyrimidin-4-yl)amino]piperidine-1-carboxylate as yellow solid (180 mg; 90.43%).

6-(4-((3-cyanomorpholino)methyl)phenyl)-4-(((S)-piperidin-3-yl)amino)pyrido[3,2-d]pyrimidine-8-carboxamide: To a stirred mixture of tert-butyl (3S)-3-[(8-carbamoyl-6-{4-[(3-cyanomorpholin-4-yl)methyl]phenyl}pyrido[3,2-d]pyrimidin-4-yl)amino]piperidine-1-carboxylate (180.00 mg; 0.21 mmol) and TFA (2 ml) in DCM (6 ml) at room temperature. The resulting mixture was stirred for 1 h at room temperature. The solvent was removed under vacuum, the residue was purified by silica gel column chromatography, eluted with CH₂Cl₂/MeOH (4:1). The crude product was purified by Prep-HPLC(Column: XBridge Prep OBD C18 Column, 30×150 mm 5 μm; Mobile Phase A:Water (10 mmoL/L NH₄HCO₃+0.1% [28% NH₃ in H₂O]), Mobile Phase B:ACN; Flow rate: 60 mL/min.; Gradient: 30 B to 60 B in 8 min.; 254 nm; RT1:6.5; RT2: 7.2; Injection Volume: 1 ml; Number Of Runs: 2 to afford 6-{4-[(3-cyanomorpholin-4-yl)methyl]phenyl}-4-{[(3S)-piperidin-3-yl]amino}pyrido[3,2-d]pyrimidine-8-carboxamide as yellow solid (24.00 mg, 23.23%).

6-(4-(((S)-3-cyanomorpholino)methyl)phenyl)-4-(((S)-piperidin-3-yl)amino)pyrido[3,2-d]pyrimidine-8-carboxamide and 6-(4-(((R)-3-cyanomorpholino)methyl)phenyl)-4-(((S)-piperidin-3-yl)amino)pyrido[3,2-d]pyrimidine-8-carboxamide: 6-{4-[(3-cyanomorpholin-4-yl)methyl]phenyl}-4-{[(3S)-piperidin-3-yl]amino}pyrido[3,2-d]pyrimidine-8-carboxamide (44.00 mg; 0.09 mmol) was separated by Prep-Chiral-HPLC(Column: CHIRALPAK IC, 3*25 cm, 5 μm; Mobile Phase A: Hex:DCM=1:1 (0.5% 2M NH₃-MeOH)-HPLC, Mobile Phase B: EtOH-HPLC; Flow rate: 40 mL/min.; Gradient: 50 B to 50 B in 20 min; 220/254 nm; RT1:13; RT2:17.9; Injection Volume: 0.5 ml; Number Of Runs: 1) to afford 6-(4-{[(3S)-3-cyanomorpholin-4-yl]methyl}phenyl)-4-{[(3S)-piperidin-3-yl]amino}pyrido[3,2-d]pyrimidine-8-carboxamide as yellow solid (15.30 mg) and 6-(4-{[(3R)-3-cyanomorpholin-4-yl]methyl}phenyl)-4-{[(3S)-piperidin-3-yl]amino}pyrido[3,2-d]pyrimidine-8-carboxamide (14.00 mg).

Isomer 1: HPLC 93.42% purity, 4.02 min. MS: m/z=473.20 [M+H]⁺. ¹H NMR (400 MHz, DMSO-d6, ppm): δ 10.04 (d, J=3.8 Hz, 1H), 8.84 (s, 1H), 8.58 (s, 1H), 8.37 (dd, J=11.9, 8.5 Hz, 3H), 8.24 (d, J=3.8 Hz, 1H), 7.54 (d, J=8.0 Hz, 2H), 4.31 (s, 1H), 4.00-3.90 (m, 2H), 3.86-3.78 (m, 1H), 3.72 (s, 2H), 3.64 (dd, J=11.7, 2.6 Hz, 1H), 3.51 (td, J=11.5, 2.7 Hz, 1H), 3.05 (d, J=11.9 Hz, 1H), 2.83 (d, J=10.9 Hz, 1H), 2.74 (t, J=10.3 Hz, 1H), 2.66-2.52 (m, 2H), 2.45 (dd, J=11.9, 3.4 Hz, 1H), 1.90 (s, 1H), 1.82 (d, J=9.8 Hz, 1H), 1.68 (s, 1H), 1.50 (d, J=12.9 Hz, 1H).

Isomer 2: HPLC 92.36% purity, 3.99 min. MS: m/z=473.15 [M+H]⁺. ¹H NMR (400 MHz, DMSO-d6, ppm): δ 10.04 (d, J=3.6 Hz, 1H), 8.85 (s, 1H), 8.58 (s, 1H), 8.37 (dd, J=14.2, 8.5 Hz, 3H), 8.24 (d, J=3.8 Hz, 1H), 7.55 (d, J=8.1 Hz, 2H), 4.32 (s, 1H), 4.00-3.91 (m, 2H), 3.82 (d, J=10.9 Hz, 1H), 3.72 (s, 2H), 3.64 (dd, J=11.7, 2.6 Hz, 1H), 3.51 (td, J=11.5, 2.7 Hz, 1H), 3.07 (d, J=12.2 Hz, 1H), 2.84 (s, 1H), 2.76 (t, J=10.2 Hz, 1H), 2.66-2.52 (m, 2H), 2.51-2.41 (m, 1H), 1.91 (s, 1H), 1.82 (d, J=10.1 Hz, 1H), 1.69 (s, 1H), 1.52 (s, 1H).

### Example 15: Synthesis of Compound 15 4-(((S)-piperidin-3-yl)amino)-6-(4-(((S)-3-(trifluoromethyl)morpholino)methyl)phenyl)pyrido[3,2-d]pyrimidine-8-carboxamide and 4-(((S)-piperidin-3-yl)amino)-6-(4-(((R)-3-(trifluoromethyl)morpholino)methyl)phenyl)pyrido[3,2-d]pyrimidine-8-carboxamide

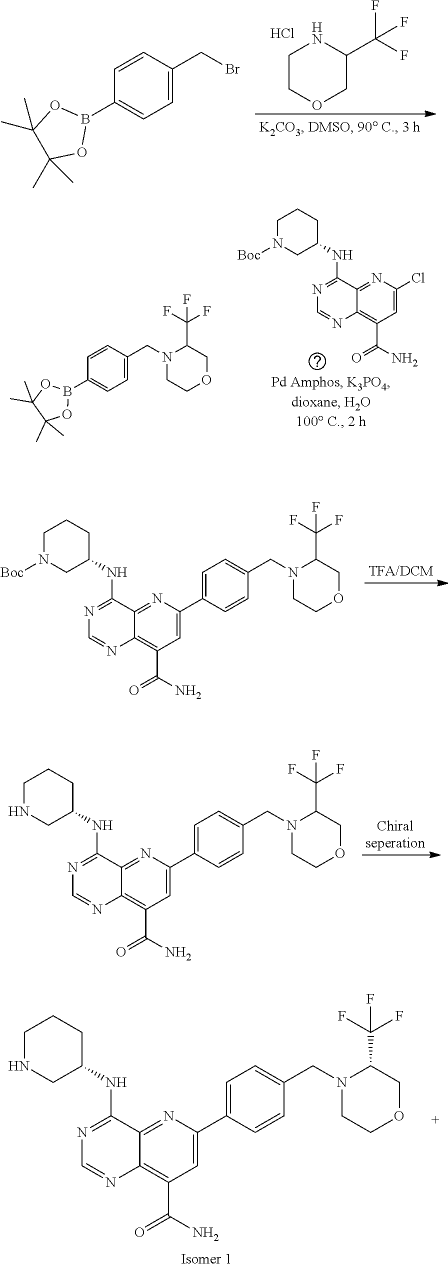

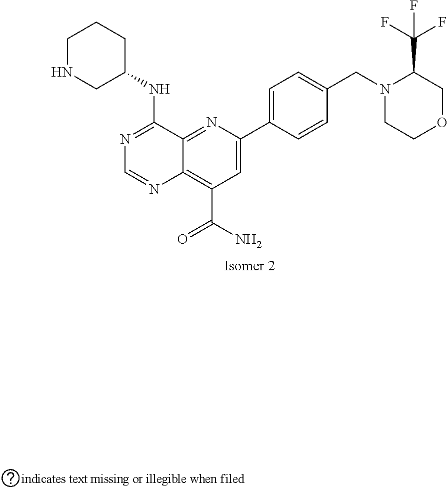 [SMILES: NC(=O)c1cc(-c2ccc(CN3CCOC[C@@H]3C(F)(F)F)cc2)nc2c(N[C@H]3CCCNC3)ncnc12]

4-(4-(4,4,5,5-tetramethyl-1,3,2-dioxaborolan-2-yl)benzyl)-3-(trifluoromethyl)morpholine: To a stirred mixture of 2-[4-(bromomethyl)phenyl]-4,4,5,5-tetramethyl-1,3,2-dioxaborolane (500.00 mg; 1.60 mmol) and 3-(trifluoromethyl)morpholine; 4-methylbenzene-1-sulfonic acid (661.26 mg; 1.92 mmol) in CH₃CN (10.00 ml) was added K₂CO₃ (698.02 mg; 4.80 mmol). The resulting mixture was stirred for 2 h at 80° C. The resulting mixture was quenched by water and extracted with EtOAc. The combined organic layers were washed with brine, dried over anhydrous Na₂SO₄. After filtration, the filtrate was concentrated under reduced pressure. This resulted in 4-{[4-(4,4,5,5-tetramethyl-1,3,2-dioxaborolan-2-yl)phenyl]methyl}-3-(trifluoromethyl)morpholine as yellow solid (450.00 mg, 62.38%).

tert-butyl(3S)-3-((8-carbamoyl-6-(4-((3-(trifluoromethyl)morpholino)methyl)phenyl)pyrido[3,2-d]pyrimidin-4-yl)amino)piperidine-1-carboxylate: To a stirred mixture of tert-butyl (3S)-3-({8-carbamoyl-6-chloropyrido[3,2-d]pyrimidin-4-yl}amino)piperidine-1-carboxylate (390.00 mg; 0.91 mmol) and 4-{[4-(4,4,5,5-tetramethyl-1,3,2-dioxaborolan-2-yl)phenyl]methyl}-3-(trifluoromethyl)morpholine (438.27 mg; 1.09 mmol) in 1,4-Dioxane (15 ml) and water (3 ml) were added K₃PO₄ (610.64 mg; 2.73 mmol) and Bis(di-tert-butyl(4-dimethylaminophenyl)phosphine)dichloropalladium(II) (67.92 mg; 0.09 mmol) under argon atmosphere. The resulting mixture was stirred for 2 h at 100° C. The residue was purified by silica gel column chromatography, eluted with PE/EtOAc (1:9) to afford tert-butyl (3S)-3-{[8-carbamoyl-6-(4-{[3-(trifluoromethyl)morpholin-4-yl]methyl}phenyl)pyrido[3,2-d]pyrimidin-4-yl]amino}piperidine-1-carboxylate as yellow solid (387.00 mg; 57.1%).

4-(((S)-piperidin-3-yl)amino)-6-(4-((3-(trifluoromethyl)morpholino)methyl)phenyl)pyrido[3,2-d]pyrimidine-8-carboxamide: To a solution of tert-butyl (3S)-3-{[8-carbamoyl-6-(4-{[3-(trifluoromethyl)morpholin-4-yl]methyl}phenyl)pyrido[3,2-d]pyrimidin-4-yl]amino}piperidine-1-carboxylate (377.00 mg; 0.51 mmol) in DCM (15 ml) were added TFA (5 ml) at room temperature. After stirring for 1 h. The solvent was removed under vacuum and the crude product was purified by Prep-HPLC(Column: XBridge Prep OBD C18 Column, 30×150 mm 5 μm; Mobile Phase A: Water (10 mmoL/L NH₄HCO₃+0.1% [28% NH₃ in H₂O]), Mobile Phase B:ACN; Flow rate: 60 mL/min.; Gradient: 20 B to 50 B in 8 min.; 254 nm; RT1:6.4; RT2: 7.8) to afford 4-{[(3S)-piperidin-3-yl]amino}-6-(4-{[3-(trifluoromethyl)morpholin-4-yl]methyl}phenyl)pyrido[3,2-d]pyrimidine-8-carboxamide as yellow solid (120.00 mg; 45.46%).

4-(((S)-piperidin-3-yl)amino)-6-(4-(((S)-3-(trifluoromethyl)morpholino)methyl)phenyl)pyrido[3,2-d]pyrimidine-8-carboxamide and 4-(((S)-piperidin-3-yl)amino)-6-(4-(((R)-3-(trifluoromethyl)morpholino)methyl)phenyl)pyrido[3,2-d]pyrimidine-8-carboxamide: The product 4-{[(3S)-piperidin-3-yl]amino}-6-(4-{[(3S)-3-(trifluoromethyl)morpholin-4-yl]methyl}phenyl)pyrido[3,2-d]pyrimidine-8-carboxamide (120.00 mg; 0.23 mmol) was separated by Prep-Chiral-HPLC(Column: CHIRAL ART Cellulose-SC, 2*25 cm, 5 um; Mobile Phase A:Hex:DCM=3:1 (0.5% 2M NH3-MeOH)-HPLC, Mobile Phase B:IPA-HPLC; Flow rate: 20 mL/min.; Gradient: 20 B to 20 B in 19 min.; 220/254 nm; RT1:15.05; RT2:17.883; Injection Volume: 0.5 ml; Number Of Runs: 6) to afford 4-{[(3S)-piperidin-3-yl]amino}-6-(4-{[(3S)-3-(trifluoromethyl)morpholin-4-yl]methyl}phenyl)pyrido[3,2-d]pyrimidine-8-carboxamide as yellow solid (68.40 mg) and 4-{[(3S)-piperidin-3-yl]amino}-6-(4-{[(3R)-3-(trifluoromethyl)morpholin-4-yl]methyl}phenyl)pyrido[3,2-d]pyrimidine-8-carboxamide as yellow solid (64.80 mg).

Isomer 1: HPLC 98.92% purity, 3.78 min. MS: m/z=516.20 [M+H]⁺. ¹H NMR (300 MHz, DMSO-d6, ppm): δ 10.03 (d, J=3.7 Hz, 1H), 8.82 (s, 1H), 8.55 (s, 1H), 8.33 (dd, J=15.1, 8.5 Hz, 3H), 8.23 (d, J=3.7 Hz, 1H), 7.53 (d, J=8.1 Hz, 2H), 4.05 (d, J=14.1 Hz, 1H), 4.00-3.85 (m, 3H), 3.81-3.66 (m, 2H), 3.54 (dd, J=11.2, 8.1 Hz, 2H), 3.09-2.99 (m, 1H), 2.92 (d, J=12.6 Hz, 1H), 2.87-2.66 (m, 2H), 2.57 (d, J=9.7 Hz, 1H), 2.40 (d, J=12.2 Hz, 1H), 1.88 (s, 2H), 1.79 (d, J=9.6 Hz, 1H), 1.67 (d, J=12.1 Hz, 1H).

Isomer 2: HPLC 99.08% purity, 3.77 min. MS:m/z=516.20 [M+H]⁺.

¹H NMR (300 MHz, DMSO-d6, ppm): δ 10.03 (d, J=3.8 Hz, 1H), 8.82 (s, 1H), 8.55 (s, 1H), 8.34 (dd, J=15.6, 8.4 Hz, 3H), 8.22 (d, J=3.7 Hz, 1H), 7.53 (d, J=8.1 Hz, 2H), 4.05 (d, J=14.1 Hz, 1H), 4.00-3.85 (m, 1H), 3.76 (d, J=12.1 Hz, 2H), 3.76-3.65 (m, 2H), 3.54 (dd, J=10.8, 7.8 Hz, 2H), 3.04 (dd, J=11.5, 3.8 Hz, 1H), 2.97-2.66 (m, 3H), 2.55 (t, J=10.0 Hz, 1H), 2.40 (d, J=12.3 Hz, 1H), 1.83 (dd, J=22.4, 7.4 Hz, 2H), 1.66 (s, 1H), 1.49 (d, J=11.2 Hz, 1H).

### Example 16: Synthesis of Compound 16 ((R)-4-((4,4-difluoropiperidin-3-yl)amino)-6-(4-((1-hydroxycyclopropyl)methoxy)phenyl)pyrido[3,2-d]pyrimidine-8-carboxamide)

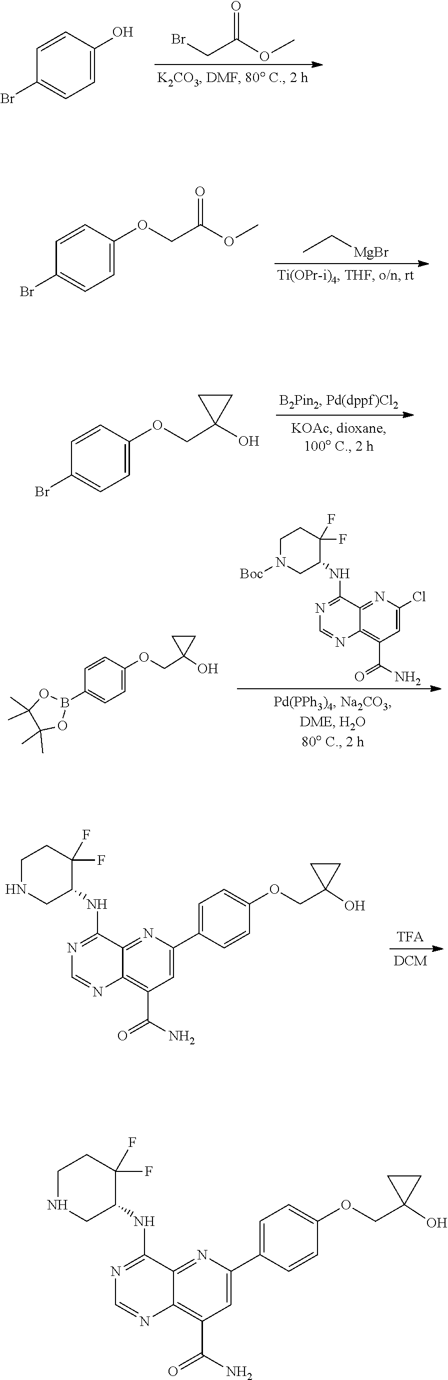

methyl 2-(4-bromophenoxy) acetate: To a stirred solution 4-bromophenol (9.50 g; 52.17 mmol) and methyl 2-bromoacetate (10.08 g; 62.60 mmol) in DMF (40.00 ml) was added K₂CO₃ (11.38 g; 78.25 mmol; 1.50 eq.) in portions at room temperature under nitrogen atmosphere. The resulting mixture was stirred for overnight at 80° C. under nitrogen atmosphere. The reaction was added 500 ml water. The mixture was extracted with EA (3×250 mL). The combined organic layers were washed with brine, dried over anhydrous Na₂SO₄. After filtration, the filtrate was concentrated under reduced pressure to afford methyl 2-(4-bromophenoxy) acetate (11.32 g; 45.66 mmol; 87.5%; brown liquid; Crude Product).

1-((4-bromophenoxy)methyl)cyclopropan-1-ol: To a stirred mixture of methyl 2-(4-bromophenoxy)acetate (8.30 g; 32.85 mmol; 1.00 eq.) in THF (80.00 ml) was added Titanium tetraisopropanolate (9.67 g; 32.32 mmol; 0.98 eq.) and bromo(ethyl)magnesium (30.60 ml) dropwise at 0° C. The resulting mixture was stirred for overnight at room temperature under nitrogen atmosphere. The reaction was quenched with 100 ml water at 0° C. and extracted with EA (3×80 mL). The combined organic layers were washed with brine and dried over anhydrous Na₂SO₄. After filtration, the filtrate was concentrated under reduced pressure. The residue was purified by silica gel column chromatography, eluted with 5% EA in PE to afford 1-[(4-bromophenoxy) methyl]cyclopropan-1-ol (3.10 g; 32.5%) as a white solid.

1-((4-(4,4,5,5-tetramethyl-1,3,2-dioxaborolan-2-yl)phenoxy)methyl)cyclopropan-1-ol: To a stirred mixture of 1-[(4-bromophenoxy)methyl]cyclopropan-1-ol (3.00 g; 10.32 mmol) and BPD (9.41 g; 35.20 mmol) in 1,4-Dioxane (20.00 ml) was added KOAc (3.65 g; 35.33 mmol; 3.42 eq.) and Pd(dppf)Cl₂ (0.91 g; 1.18 mmol; 0.11 eq.) in portions at room temperature. The resulting mixture was stirred for 1 h at 100° C. under nitrogen atmosphere. The residue was purified by silica gel column chromatography, and eluted with 10% EA in PE. The residue was purified by reverse flash chromatography with the following conditions: column, C18 silica gel; mobile phase, ACN in water, 45% to 55% gradient in 10 min; detector, UV 254 nm to afford 1-{[4-(4,4,5,5-tetramethyl-1,3,2-dioxaborolan-2-yl) phenoxy]methyl}cyclopropan-1-ol (650.00 mg 21.7%) as a yellow oil.

tert-butyl (R)-3-((8-carbamoyl-6-(4-((1-hydroxycyclopropyl)methoxy)phenyl)pyrido[3,2-d]pyrimidin-4-yl)amino)-4,4-difluoropiperidine-1-carboxylate: To a stirred mixture of tert-butyl (3R)-3-({8-carbamoyl-6-chloropyrido[3,2-d]pyrimidin-4-yl}amino)-4,4-difluoropiperidine-1-carboxylate (280.00 mg; 0.61 mmol) and 1-{[4-(4,4,5,5-tetramethyl-1,3,2-dioxaborolan-2-yl)phenoxy]methyl}cyclopropan-1-ol (460.00 mg; 1.59 mmol) in DME (3.00 ml) and H₂O (1.00 ml) was added Na₂CO₃ (269.00 mg; 2.41 mmol) and Pd(PPh₃)₄ (74.00 mg; 0.06 mmol) in portions at room temperature. The resulting mixture was stirred for 1 h at 80° C. under nitrogen atmosphere. The reaction mixture was concentrated under reduced pressure. The residue was purified by silica gel column chromatography, eluted with 45% EA in PE to afford tert-butyl (3R)-3-[(8-carbamoyl-6-{4-[(1-hydroxycyclopropyl)methoxy]phenyl}pyrido[3,2-d]pyrimidin-4-yl)amino]-4,4-difluoropiperidine-1-carboxylate (200.00 mg; 51.7%) as a yellow solid.

(R)-4-((4,4-difluoropiperidin-3-yl)amino)-6-(4-((1-hydroxycyclopropyl)methoxy)phenyl)pyrido[3,2-d]pyrimidine-8-carboxamide: To a stirred mixture of tert-butyl (3R)-3-[(8-carbamoyl-6-{4-[(1-hydroxycyclopropyl)methoxy]phenyl}pyrido[3,2-d]pyrimidin-4-yl)amino]-4,4-difluoropiperidine-1-carboxylate (190.00 mg; 0.30 m mol; 1.00 eq.) in DCM (3.00 ml) was added TFA (1.00 ml) dropwise at 0° C. The resulting mixture was stirred for 1 h at room temperature. The reaction mixture was concentrated under reduced pressure. The residue was purified by reverse flash chromatography with the following conditions: column, C18 silica gel; mobile phase, ACN in water, 20% to 25% gradient in 10 min; detector, UV 220 nm. The product was purified by Prep-HPLC with the following conditions (Column: X Bridge Prep OBD C18 Column, 30×150 mm Sum; Mobile Phase A:Water (10 mmoL/L NH₄HCO₃+0.1% [28% NH₃ in H₂O]), Mobile Phase B:ACN; Flow rate: 60 mL/min.; Gradient: 18 B to 48 B in 8 min.) to afford 4-{[(3R)-4,4-difluoropiperidin-3-yl]amino}-6-{4-[(1-hydroxycyclopropyl)methoxy]phenyl}pyrido[3,2-d]pyrimidine-8-carboxamide (35.40 mg; 0.07 m mol; 23.7%) as a yellow solid.

HPLC: 93.7% purity, RT=3.20 min. MS: m/z=471.10 [M+H]⁺. ¹H NMR (300 MHz, DMSO-d6) δ 9.90 (s, 1H), 8.82 (s, 1H), 8.62 (s, 1H), 8.30 (d, J=8.4 Hz, 2H), 8.22 (s, 1H), 8.16 (d, J=9.8 Hz, 1H), 7.13 (d, J=8.4 Hz, 2H), 5.63 (s, 1H), 4.88 (s, 1H), 4.07 (s, 2H), 3.07 (s, 1H), 2.97 (d, J=11.2 Hz, 2H), 2.73 (s, 1H), 2.62 (s, 1H), 2.14 (s, 1H), 1.96 (s, 1H), 0.68 (d, J=13.6 Hz, 4H).

### Example 17: Synthesis of Compound 17 6-(4-morpholinocyclohex-1-en-1-yl)-4-(((S)-piperidin-3-yl)amino)pyrido[3,2-d]pyrimidine-8-carboxamide

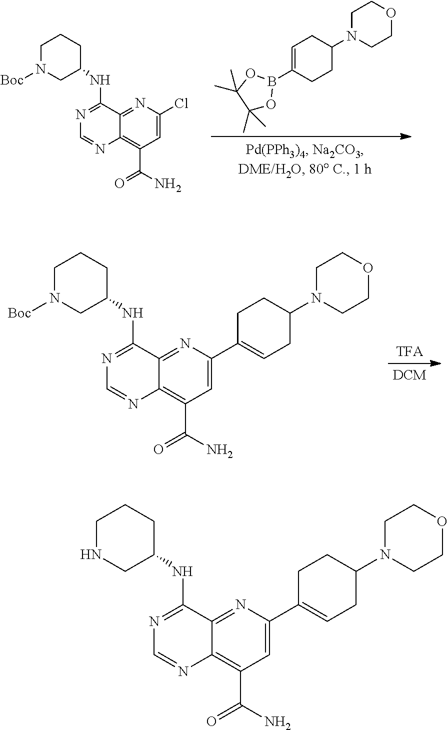

tert-butyl (3S)-3-((8-carbamoyl-6-(4-morpholinocyclohex-1-en-1-yl)pyrido[3,2-d]pyrimidin-4-yl)amino)piperidine-1-carboxylate: To a stirred mixture of tert-butyl (3S)-3-({8-carbamoyl-6-chloropyrido[3,2-d]pyrimidin-4-yl}amino)piperidine-1-carboxylate (300.00 mg; 0.70 mmol) and 4-[4-(4,4,5,5-tetramethyl-1,3,2-dioxaborolan-2-yl)cyclohex-3-en-1-yl]morpholine (259.54 mg; 0.84 mmol) in 1,4-Dioxane (15 ml) and H₂O (3 ml) were added Bis(di-tert-butyl(4-dimethylaminophenyl)phosphine)dichloropalladium(II) (52.24 mg; 0.07 mmol) and K₃PO₄ (469.74 mg; 2.10 mmol) under argon atmosphere. After stirring for 2 h at 100° C. The residue was purified by silica gel column chromatography, eluted with CH₂Cl₂/MeOH (7:3) to afford tert-butyl (3S)-3-({8-carbamoyl-6-[4-(morpholin-4-yl)cyclohex-1-en-1-yl]pyrido[3,2-d]pyrimidin-4-yl}amino)piperidine-1-carboxylate yellow solid (390.00 mg, 92.0%).

6-(4-morpholinocyclohex-1-en-1-yl)-4-(((S)-piperidin-3-yl)amino)pyrido[3,2-d]pyrimidine-8-carboxamide: To a solution of tert-butyl (3S)-3-({8-carbamoyl-6-[4-(morpholin-4-yl)cyclohex-1-en-1-yl]pyrido[3,2-d]pyrimidin-4-yl}amino)piperidine-1-carboxylate (380.00 mg; 0.63 mmol) in DCM (15 ml) were added TFA (5 ml). The resulting mixture was stirred for 1 h at room temperature. The crude product was purified by Prep-HPLC (Column: XBridge Prep OBD C18 Column, 30×150 mm 5 μm; Mobile Phase A:Water (10 mmol/L NH₄HCO₃+0.1% [28% NH₃ in H₂O]), Mobile Phase B:ACN; Flow rate: 60 mL/min.; Gradient: 10 B to 40 B in 8 min.) to afford 6-[4-(morpholin-4-yl)cyclohex-1-en-1-yl]-4-{[(3S)-piperidin-3-yl]amino}pyrido[3,2-d]pyrimidine-8-carboxamide as yellow solid (107.20 mg, 38.8%)

HPLC 99.4% purity, RT=2.42 min. MS: m/z=438.25 [M+H]⁺. ¹H NMR (400 MHz, DMSO, ppm): δ 10.04 (d, J=3.8 Hz, 1H), 8.51 (d, J=8.9 Hz, 2H), 8.18 (d, J=3.8 Hz, 1H), 8.10 (d, J=8.6 Hz, 1H), 6.93 (d, J=4.3 Hz, 1H), 4.25 (d, J=8.5, 4.3 Hz, 1H), 3.60 (t, J=4.6 Hz, 4H), 3.05-2.90 (m, 2H), 2.83-2.67 (m, 2H), 2.60-2.52 (m, 4H), 2.47 (d, J=6.0 Hz, 4H), 2.35-2.20 (m, 1H), 2.11 (d, J=12.4 Hz, 1H), 1.87-1.74 (m, 2H), 1.64 (s, 1H), 1.49 (d, J=13.6, 5.0 Hz, 2H).

### Example 18: Synthesis of Compound 18 ((R)-3-amino-1-((3R,5S)-5-methyl-1-(pyrido[3,2-b]pyrazin-8-yl)piperidin-3-yl)pyrrolidin-2-one)

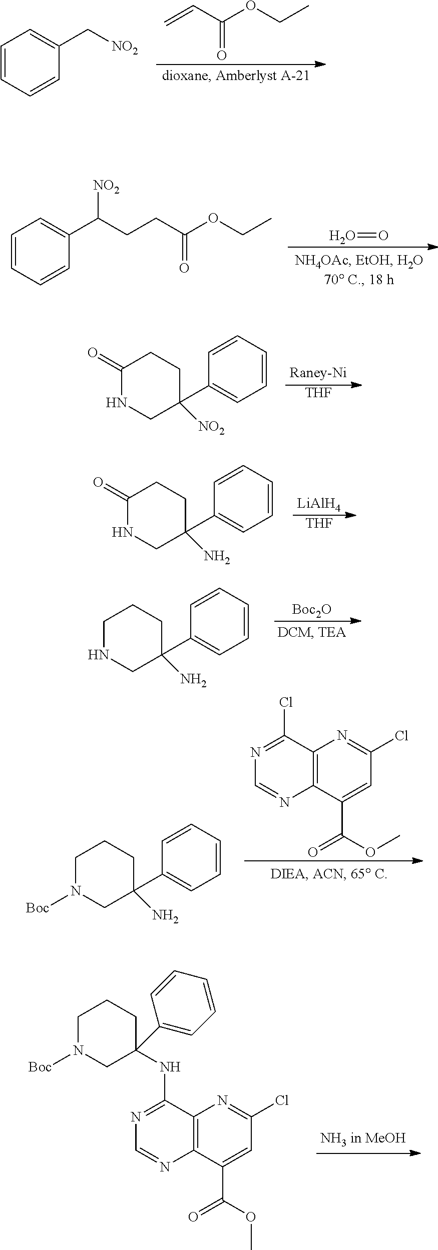

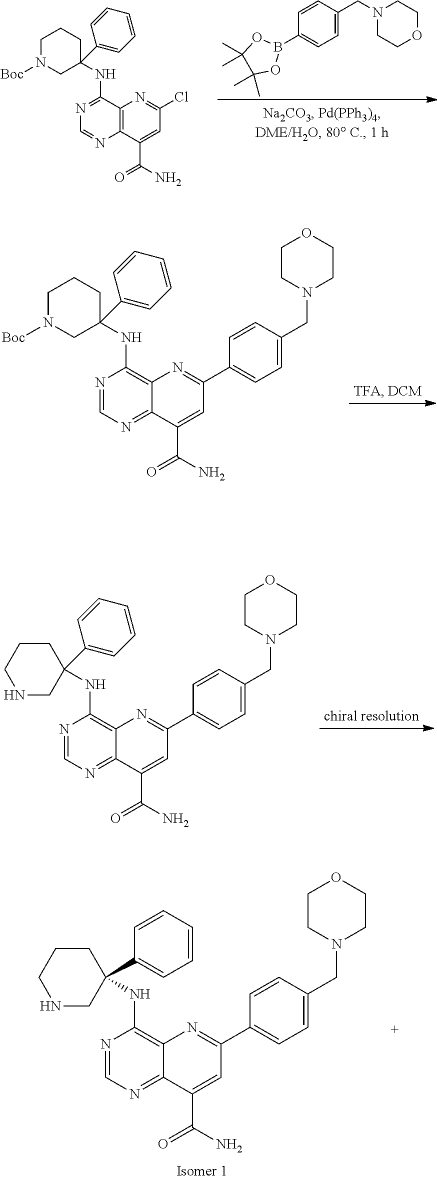 [SMILES: CC(C)(C)OC(=O)N1CCCC(Nc2ncnc3c(C(N)=O)cc(Cl)nc23)(c2ccccc2)C1 ; CC1(C)OB(c2ccc(CN3CCOCC3)cc2)OC1(C)C ; CC(C)(C)OC(=O)N1CCCC(Nc2ncnc3c(C(N)=O)cc(-c4ccc(CN5CCOCC5)cc4)nc23)(c2ccccc2)C1 ; NC(=O)c1cc(-c2ccc(CN3CCOCC3)cc2)nc2c(NC3(c4ccccc4)CCCNC3)ncnc12 ; NC(=O)c1cc(-c2ccc(CN3CCOCC3)cc2)nc2c(N[C@@]3(c4ccccc4)CCCNC3)ncnc12]

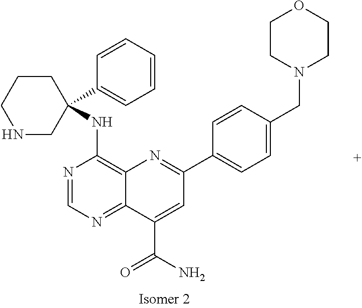 [SMILES: NC(=O)c1cc(-c2ccc(CN3CCOCC3)cc2)nc2c(N[C@]3(c4ccccc4)CCCNC3)ncnc12]

ethyl 4-nitro-4-phenyl butanoate: To a stirred solution of (nitromethyl) benzene (7.00 g, 48.491 mmol) and ethyl acrylate (5.11 g, 48.491 mmol) in 1,4-Dioxane (70.00 mL, 826.286 mmol) was added Amberlyst A-21 (21 g) at 0° C. under nitrogen atmosphere. The resulting mixture was stirred for overnight at room temperature under nitrogen atmosphere, filtered and the filtrate was dried over sodium sulfate and concentrated in vacuo. The crude product was purified with flash column chromatography on silica gel (Eluent: Heptane/ethyl acetate 0 to 10%) to afford ethyl 4-nitro-4-phenylbutanoate (8 g, 65.99%) as a colorless oil.

5-nitro-5-phenylpiperidin-2-one: To a stirred mixture of NH₄OAc (5.19 g, 63.999 mmol) in EtOH (15 mL) was added formaldehyde solution (2.60 g, 31.999 mmol) dropwise at room temperature under nitrogen atmosphere. To the above mixture was added ethyl 4-nitro-4-phenylbutanoate (8.00 g, 31.999 mmol) in EtOH (5.00 mL) dropwise at room temperature. The resulting mixture was stirred for an additional 18 h at 70° C. The resulting mixture was concentrated under reduced pressure. The residue was purified by trituration with water then Et₂O to afford 5-nitro-5-phenylpiperidin-2-one (5 g, 70.95%) as a white solid.

5-amino-5-phenylpiperidin-2-one: To a stirred solution of 5-nitro-5-phenylpiperidin-2-one (5.00 g; 21.57 mmol) in Tetrahydrofuran (30.00 ml) was added Raney Ni (2.00 g) in portions under N₂ atmosphere. The resulting mixture was stirred for overnight under H₂ atmosphere. The mixture was filtered and concentrated under reduced pressure to afford 5-amino-5-phenylpiperidin-2-one (4.10 g; 18.32 mmol) as a white solid.

3-phenylpiperidin-3-amine: To a solution of Lithium Aluminum Hydride (5.45 g; 43.10 mmol) in THF was added a solution of 5-amino-5-phenylpiperidin-2-one (4.10 g; 21.55 mmol) in Tetrahydrofuran (50.00 ml) dropwise at room temperature. The mixture was refluxed for 30 minutes. The resulting mixture was cooled in an ice bath and quenched carefully with 5.17 mL water, 5.17 mL NaOH (5N) and finally 15.51 mL water. Ethyl acetate was added. The mixture was filtered and the filtrate was concentrated in vacuum to provide 3-phenylpiperidin-3-amine (2.80 g; 13.11 mmol; 60.82%; yellow oil; Crude Product).

tert-butyl 3-amino-3-phenylpiperidine-1-carboxylate: To a stirred solution of 3-phenylpiperidin-3-amine (2.80 g; 13.11 mmol) and triethylamine (2.09 g; 19.66 mmol) in DCM (30.00 ml) was added a solution of di-tert-butyl dicarbonate (3.61 g; 15.73 mmol) in DCM at 0° C. under N₂ atmosphere. The resulting mixture was stirred for 2 h at room temperature. The resulting mixture was quenched with Na₂CO₃ (aq.). The solution was extracted with DCM and concentrated under reduced pressure. The residue was purified by C18 flash column (30% ACN in water) to afford tert-butyl 3-amino-3-phenylpiperidine-1-carboxylate (2.50 g; 57.69%) as a colorless oil.

3-{[6-chloro-8-(methoxycarbonyl)pyrido[3,2-d]pyrimidin-4-yl]amino}-3-phenylpiperidine-1-carboxylate: To a stirred mixture of methyl 4,6-dichloropyrido[3,2-d]pyrimidine-8-carboxylate (800.00 mg; 1.86 mmol) and tert-butyl 3-amino-3-phenylpiperidine-1-carboxylate (603.00 mg; 1.86 mmol) in ACN (20.00 ml) was added DIEA (0.93 ml; 5.58 mmol) at room temperature. The resulting mixture was stirred for overnight at 65° C. under nitrogen atmosphere. The resulting mixture was concentrated under vacuum. The residue was purified by silica gel column chromatography, eluted with PE:EA=7:3 to afford tert-butyl 3-{[6-chloro-8-(methoxycarbonyl)pyrido[3,2-d]pyrimidin-4-yl]amino}-3-phenylpiperidine-1-carboxylate (700.00 mg; 1.41 mmol) as a yellow solid.

tert-butyl 3-({8-carbamoyl-6-chloropyrido[3,2-d]pyrimidin-4-yl}amino)-3-phenylpiperidine-1-carboxylate: To a stirred mixture of tert-butyl 3-{[6-chloro-8-(methoxycarbonyl)py tert-butyl 3-({8-carbamoyl-6-chloropyrido[3,2-d]pyrimidin-4-yl}amino)-3-phenylpiperidine-1-carboxylaterido[3,2-d]pyrimidin-4-yl]amino}-3-phenylpiperidine-1-carboxylate (680.00 mg; 1.37 mmol) in NH₃ (g) in MeOH (15.00 ml, 13%) at room temperature. The resulting mixture was stirred for 2 h at 40° C. under nitrogen atmosphere. The resulting mixture was concentrated under vacuum. This resulted in tert-butyl 3-({8-carbamoyl-6-chloropyrido[3,2-d]pyrimidin-4-yl}amino)-3-phenylpiperidine-1-carboxylate (600.00 mg; Crude Product) as a yellow solid.

tert-butyl 3-[(8-carbamoyl-6-{4-[(morpholin-4-yl)methyl]phenyl}pyrido[3,2-d]pyrimidin-4-yl)amino]-3-phenylpiperidine-1-carboxylate: To a stirred mixture of tert-butyl 3-({8-carbamoyl-6-chloropyrido[3,2-d]pyrimidin-4-yl}amino)-3-phenylpiperidine-1-carboxylate (300.00 mg; 0.62 mmol) and 4-{[4-(4,4,5,5-tetramethyl-1,3,2-dioxaborolan-2-yl)phenyl]methyl}morpholine (396.00 mg; 1.30 mmol) in DME (12.00 ml) were added Pd(PPh₃)₄ (76.00 mg, 0.062 mmol), Na₂CO₃ (208.00 mg, 1.96 mmol) and water (4.00 ml) at room temperature. The resulting mixture was stirred for 2 h at 80° C. under nitrogen atmosphere. The residue was purified by silica gel column chromatography, eluted with PE:EA=4:6 afford tert-butyl 3-[(8-carbamoyl-6-{4-[(morpholin-4-yl)methyl]phenyl}pyrido[3,2-d]pyrimidin-4-yl)amino]-3-phenylpiperidine-1-carboxylate (300.00 mg; 77.4%) as a yellow solid.

6-{4-[(morpholin-4-yl)methyl]phenyl}-4-[(3-phenylpiperidin-3-yl)amino]pyrido[3,2-d]pyrimidine-8-carboxamide: To a stirred mixture of tert-butyl 3-[(8-carbamoyl-6-{4-[(morpholin-4-yl)methyl]phenyl}pyrido[3,2-d]pyrimidin-4-yl)amino]-3-phenylpiperidine-1-carboxylate (280.00 mg; 0.45 mmol) in DCM (8.00 ml) was added TFA (2.00 ml) at room temperature. The resulting mixture was stirred for 2 h at room temperature under nitrogen atmosphere. The reaction was quenched with NaHCO₃ (aq.) at room temperature. The resulting mixture was extracted with DCM (3×30 mL). The combined organic layers were washed with brine, dried over anhydrous Na₂SO₄. After filtration, the filtrate was concentrated under reduced pressure. The resulting mixture was concentrated under vacuum. This resulted in 6-{4-[(morpholin-4-yl)methyl]phenyl}-4-[(3-phenylpiperidin-3-yl)amino]pyrido[3,2-d]pyrimidine-8-carboxamide (200.00 mg; Crude Product) as a yellow solid.

6-{4-[(morpholin-4-yl)methyl]phenyl}-4-{[(3R)-3-phenylpiperidin-3-yl]amino}pyrido[3,2-d]pyrimidine-8-carboxamide & 6-{4-[(morpholin-4-yl)methyl]phenyl}-4-{[(3S)-3-phenylpiperidin-3-yl]amino}pyrido[3,2-d]pyrimidine-8-carboxamide: The 6-{4-[(morpholin-4-yl)methyl]phenyl}-4-[(3-phenylpiperidin-3-yl)amino]pyrido[3,2-d]pyrimidine-8-carboxamide (200.00 mg; 0.37 mmol) was purified by Column: CHIRAL ART Cellulose-SB, 2*25 cm, 5 μm; Mobile Phase A:Hex (0.5% 2M NH₃-MeOH)-HPLC, Mobile Phase B: EtOH-HPLC; Flow rate: 20 mL/min.; Gradient: 50 B to 50 B in 15 min.; 254/220 nm; RT1:10.9; RT2:12.3; Injection Volume: 0.3 ml; Number Of Runs: 2; to afford 6-{4-[(morpholin-4-yl)methyl]phenyl}-4-{[(3R)-3-phenylpiperidin-3-yl]amino}pyrido[3,2-d]pyrimidine-8-carboxamide (41.50 mg; 41.8%) as a yellow solid and 6-{4-[(morpholin-4-yl)methyl]phenyl}-4-{[(3S)-3-phenylpiperidin-3-yl]amino}pyrido[3,2-d]pyrimidine-8-carboxamide (40.80 mg; 40.8%) as a yellow solid.

Isomer 1: 6-{4-[(morpholin-4-yl)methyl]phenyl}-4-{[(3R)-3-phenylpiperidin-3-yl]amino}pyrido[3,2-d]pyrimidine-8-carboxamide. HPLC: 98.08% purity, RT=4.38 min. MS: m/z=524.25 [M+H]⁺. ¹H NMR (400 MHz, DMSO-d6) δ 9.92 (d, J=3.6 Hz, 1H), 8.86 (d, J=3.0 Hz, 2H), 8.32 (s, 1H), 8.31-8.25 (m, 2H), 8.25-8.19 (m, 1H), 7.58-7.52 (m, 2H), 7.52-7.45 (m, 2H), 7.31 (t, J=7.7 Hz, 2H), 7.27-7.18 (m, 1H), 3.60 (dd, J=10.0, 5.3 Hz, 6H), 3.25 (d, J=11.7 Hz, 1H), 2.95 (d, J=11.4 Hz, 2H), 2.69 (d, J=11.9 Hz, 1H), 2.59 (s, 1H), 2.42 (t, J=4.6 Hz, 4H), 2.18-2.06 (m, 1H), 1.53 (s, 2H), 1.24 (s, 1H).

Isomer 2: 6-{4-[(morpholin-4-yl)methyl]phenyl}-4-{[(3S)-3-phenylpiperidin-3-yl]amino}pyrido[3,2-d]pyrimidine-8-carboxamide. HPLC: 97.3% purity, RT=4.37 min. MS: m/z=524.25 [M+H]⁺. ¹H NMR (400 MHz, DMSO-d6) δ 9.92 (d, J=3.6 Hz, 1H), 8.87 (s, 1H), 8.83 (s, 1H), 8.33 (s, 1H), 8.31-8.25 (m, 2H), 8.22 (d, J=3.6 Hz, 1H), 7.58-7.51 (m, 2H), 7.53-7.45 (m, 2H), 7.31 (dd, J=8.4, 6.9 Hz, 2H), 7.27-7.18 (m, 1H), 3.60 (dd, J=10.4, 5.7 Hz, 6H), 2.97 (t, J=15.6 Hz, 2H), 2.74 (d, J=11.9 Hz, 1H), 2.61 (d, J=9.4 Hz, 1H), 2.41 (t, J=4.7 Hz, 4H), 2.12 (dt, J=13.2, 8.7 Hz, 1H), 1.56 (s, 2H), 1.24 (d, J=13.0 Hz, 2H).

### Example 19: Synthesis of Compound 19 (S)-6-(1-(2-methoxyethyl)-1H-pyrazol-4-yl)-4-(piperidin-3-ylamino)pyrido[3,2-d]pyrimidine-8-carboxamide

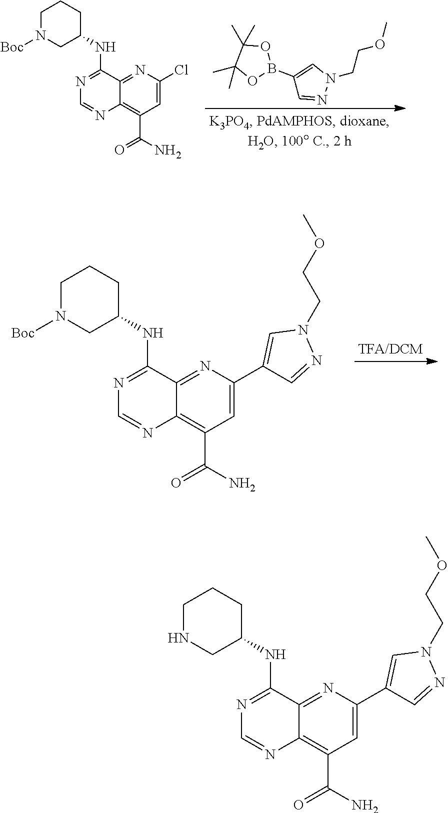

tert-butyl (S)-3-((8-carbamoyl-6-(1-(2-methoxyethyl)-1H-pyrazol-4-yl)pyrido[3,2-d]pyrimidin-4-yl)amino)piperidine-1-carboxylate: To a stirred mixture of 1-(2-methoxyethyl)-4-(4,4,5,5-tetramethyl-1,3,2-dioxaborolan-2-yl)pyrazole (150 mg, 0.507 mmol) and tert-butyl 3-([8-carbamoyl-6-chloropyrido[3,2-d]pyrimidin-4-yl]amino)piperidine-1-carboxylate (325 mg, 0.760 mmol) in 1,4-Dioxane and H₂O (3 mL) was added K₃PO₄ (339 mg, 1.521 mmol) and AMPHOS-PdCl₂ (37 mg, 0.051 mmol) at 25° C. The resulting mixture was stirred for 2 h at 100° C. under argon atmosphere. The solvent was removed under vacuum and the residue was purified by silica gel column chromatography, eluted with CH₂Cl₂/MeOH (6:1) to afford tert-butyl (3S)-3-([8-carbamoyl-6-[1-(2-methoxyethyl)pyrazol-4-yl]pyrido[3,2-d]pyrimidin-4-yl]amino)piperidine-1-carboxylate (120 mg, 38.09%) as yellow oil.

(S)-6-(1-(2-methoxyethyl)-1H-pyrazol-4-yl)-4-(piperidin-3-ylamino)pyrido[3,2-d]pyrimidine-8-carboxamide: To a stirred mixture of tert-butyl (3S)-3-([8-carbamoyl-6-[1-(2-methoxyethyl)pyrazol-4-yl]pyrido[3,2-d]pyrimidin-4-yl]amino)piperidine-1-carboxylate (110 mg, 0.177 mmol) in DCM was added TFA (4 mL, 51.160 mmol) at 25° C. The resulting mixture was stirred for 1 h at 25° C. The solvent was removed under vacuum and the crude product was purified by Prep-HPLC with the following conditions (2#SHIMADZU (HPLC-01)): Column, XBridge Prep OBD C18 Column, 30 150 mm 5 μm; mobile phase, Water (10 mmol/L NH₄HCO₃+0.1% [28% NH₃ in H₂O]) and ACN (20% Phase B up to 50% in 8 min.); Detector, UV 254 nm. This resulted in 6-[1-(2-methoxyethyl)pyrazol-4-yl]-4-[(3S)-piperidin-3-ylamino]pyrido[3,2-d]pyrimidine-8-carboxamide (50.8 mg, 71.82%) as a yellow solid.

HPLC 99.2% purity, 1.987 min. MS: m/z=397.4 [M+H]⁺. ¹H NMR (300 MHz, DMSO-d6, ppm): δ 10.04 (d, J=3.6 Hz, 1H), 8.79-8.65 (m, 1H), 8.55 (s, 1H), 8.51 (s, 1H), 8.40 (d, J=0.7 Hz, 1H), 8.17 (d, J=8.6 Hz, 2H), 4.40-4.14 (m, 3H), 3.77 (t, J=5.2 Hz, 2H), 3.31 (s, 2H), 3.07 (dd, J=11.5, 3.8 Hz, 1H), 2.84 (d, J=12.2 Hz, 1H), 2.76-2.55 (m, 2H), 1.93 (s, 1H), 1.86-1.64 (m, 2H), 1.49 (d, J=11.6 Hz, 1H).

### Example 20: Synthesis of Compound 20 4-((3-azabicyclo[3.1.0]hexan-1-yl)amino)-6-(4-(morpholinomethyl)phenyl)pyrido[3,2-d]pyrimidine-8-carboxamide

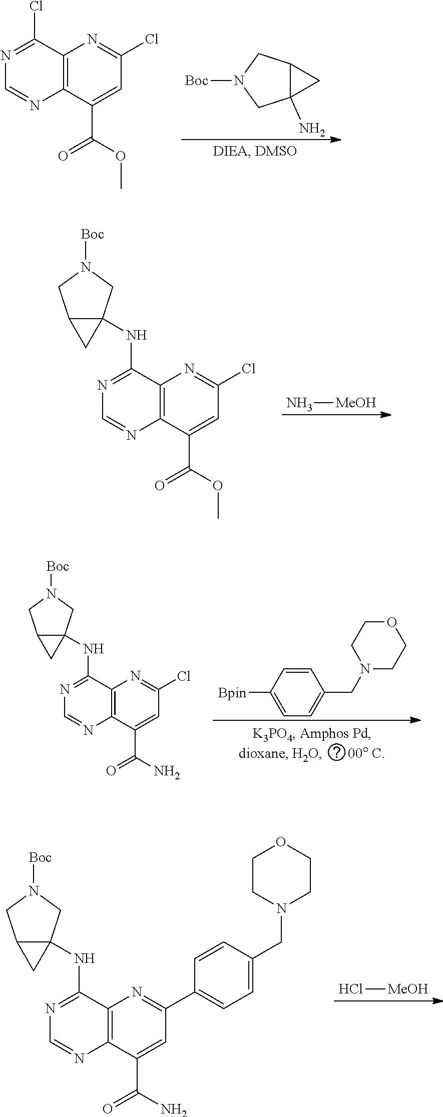

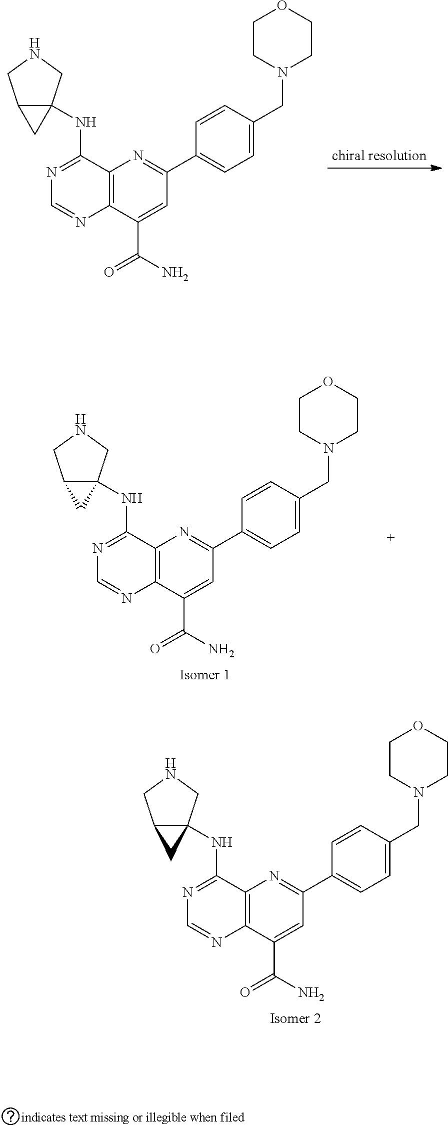 [SMILES: NC(=O)c1cc(-c2ccc(CN3CCOCC3)cc2)nc2c(NC34CNCC3C4)ncnc12 ; NC(=O)c1cc(-c2ccc(CN3CCOCC3)cc2)nc2c(N[C@]34CNC[C@H]3C4)ncnc12 ; NC(=O)c1cc(-c2ccc(CN3CCOCC3)cc2)nc2c(N[C@@]34CNC[C@@H]3C4)ncnc12]

methyl 4-((3-(tert-butoxycarbonyl)-3-azabicyclo[3.1.0]hexan-1-yl)amino)-6-chloropyrido[3,2-d]pyrimidine-8-carboxylate: A mixture of methyl 4,6-dichloropyrido[3,2-d]pyrimidine-8-carboxylate (200 mg, 0.660 mmol) and tert-butyl 1-aminobicyclo[3.1.0]hexane-3-carboxylate (274.24 mg, 1.321 mmol) in MeCN (5 mL) was added DIEA (0.36 mL, 2.810 mmol). The resulting mixture was stirred for 16 h at 40° C. under nitrogen atmosphere. The solvent was removed, and the residue was purified by silica gel column chromatography, eluted with PE/EtOAc (3:17) to afford methyl 4-[[3-(tert-butoxycarbonyl)bicyclo[3.1.0]hexan-1-yl]amino]-6-chloropyrido[3,2-d]pyrimidine-8-carboxylate (142 mg, 47.54%) as a yellow solid.

tert-butyl 1-((8-carbamoyl-6-chloropyrido[3,2-d]pyrimidin-4-yl)amino)-3-azabicyclo[3.1.0]hexane-3-carboxylate: A solution of tert-butyl 1-[[6-chloro-8-(methoxycarbonyl)pyrido[3,2-d]pyrimidin-4-yl]amino]-3-azabicyclo[3.1.0]hexane-3-carboxylate (142.00 mg, 0.313 mmol) in NH₃ (g) in MeOH (3 mL) was stirred for 2 h at 40° C. under nitrogen atmosphere. The resulting mixture was concentrated under vacuum. This resulted in tert-butyl 1-([8-carbamoyl-6-chloropyrido[3,2-d]pyrimidin-4-yl]amino)-3-azabicyclo[3.1.0]hexane-3-carboxylate (130 mg, crude) as a light-yellow solid used in the next step without purification.

tert-butyl 1-((8-carbamoyl-6-(4-(morpholinomethyl)phenyl)pyrido[3,2-d]pyrimidin-4-yl)amino)-3-azabicyclo[3.1.0]hexane-3-carboxylate: To a solution of tert-butyl 1-([8-carbamoyl-6-chloropyrido[3,2-d]pyrimidin-4-yl]amino)-3-azabicyclo[3.1.0]hexane-3-carboxylate (120 mg, 0.250 mmol) and 4-[[4-(4,4,5,5-tetramethyl-1,3,2-dioxaborolan-2-yl)phenyl]methyl]morpholine (95.58 mg, 0.299 mmol) in 1,4-Dioxane (5 mL) and H₂O (1 ml) were added K₃PO₄ (167.29 mg, 0.749 mmol) and AMPHOS-PdCl₂ (18.60 mg, 0.025 mmol). The resulting mixture was stirred for 2 h at 100° C. under nitrogen atmosphere, the resulting mixture was concentrated under reduced pressure. The residue was purified by Prep-TLC/silica gel column chromatography, eluted with CH₂Cl₂/MeOH (9:1) to afford tert-butyl 1-([8-carbamoyl-6-[4-(morpholin-4-ylmethyl)phenyl]pyrido[3,2-d]pyrimidin-4-yl]amino)-3-azabicyclo[3.1.0]hexane-3-carboxylate (100 mg, 57.87%) as a yellow solid.

4-((3-azabicyclo[3.1.0]hexan-1-yl)amino)-6-(4-(morpholinomethyl)phenyl)pyrido[3,2-d]pyrimidine-8-carboxamide: To a stirred solution of tert-butyl 1-([8-carbamoyl-6-[4-(morpholin-4-ylmethyl)phenyl]pyrido[3,2-d]pyrimidin-4-yl]amino)-3-azabicyclo[3.1.0]hexane-3-carboxylate (90 mg, 0.130 mmol) and HCl (g) in MeOH (5 mL) under air atmosphere. The resulting mixture was concentrated under vacuum. The crude product (85 mg) was purified by Prep-HPLC with the following conditions (2#SHIMADZU (HPLC-01)): Column, XBridge Prep OBD C18 Column, 150 mm 5 μm; mobile phase, water (10 mmoL/L NH₄HCO₃+0.1% [28% NH₃ in H₂O]) and ACN (26% PhaseB up to 56% in 8 min.); Detector, UV 254 nm. Product was obtained. This resulted in 4-[3-azabicyclo[3.1.0]hexan-1-ylamino]-6-[4-(morpholin-4-ylmethyl)phenyl]pyrido[3,2-d]pyrimidine-8-carboxamide (35.8 mg, 60.46%) as a light-yellow solid.

HPLC 97.8% purity, RT=2.11 min. MS: m/z=446.1 [M+H]⁺. ¹H NMR (300 MHz, DMSO-d6, ppm): δ 10.03 (d, J=3.7 Hz, 1H), 9.17 (s, 1H), 8.83 (s, 1H), 8.59 (s, 1H), 8.42-8.33 (m, 2H), 8.22 (d, J=3.7 Hz, 1H), 7.47 (d, J=8.1 Hz, 2H), 3.57 (dd, J=10.1, 5.5 Hz, 6H), 3.20-3.04 (m, 2H), 2.84 (d, J=10.5 Hz, 1H), 2.76 (d, J=11.2 Hz, 1H), 2.37 (t, J=4.7 Hz, 4H), 1.73 (dt, J=8.3, 4.3 Hz, 1H), 1.10 (t, J=5.1 Hz, 1H), 0.95 (dd, J=8.4, 5.3 Hz, 1H).

4-({3-azabicyclo[3.1.0]hexan-1-yl}amino)-6-{4-[(morpholin-4-yl)methyl]phenyl}pyrido[3,2-d]pyrimidine-8-carboxamide (35.1 mg; 0.079 mmol) was dissolved in a minimal amount of DMSO and was purified using super critical fluid chromatography. The purification was run on a Waters SFC Prep 200 (Phenomenex Cellulose-3, 250×21 mm, particle size of 5 um, pore size 1000 A) with a column temperature of 40° C. and mobile phases A: Methanol+20 mM NH₄OH B: CO₂. The desired enantiomers were isolated at 45% solvent A and 55% solvent B at an isocratic flow rate of 100 g/min., fractions containing product were combined and concentrated under vacuum. Enantiomeric excess was determined on an Agilent 1100 with an Aurora Fusion A5 SFC (Phenomenex Cellulose-3, 4.6×150 mm, particle size of 5 μm, pore size 1000 A) with a column temperature of 40° C. and mobile phases A: CO₂ B: Methanol+20 mM NH₄OH. The enantiomers were chromatographed using a gradient of 95% solvent A+5% solvent B to 40% solvent A+60% solvent B over 3.5 minutes, isocratic at 40% solvent A+60% solvent B 3.5-5 min, isocratic at 95% solvent A+5% solvent B 5-6 min. at a flow of 3 mL/min. Chirality was arbitrarily assigned with the first peak as 4-{[(1R,5S)-3-azabicyclo[3.1.0]hexan-1-yl]amino}-6-{4-[(morpholin-4-yl)methyl]phenyl}pyrido[3,2-d]pyrimidine-8-carboxamide (5.2 mg, 98% enantiomeric purity) with a RT 3.251 min, and peak two was assigned as 4-{[(1S,5R)-3-azabicyclo[3.1.0]hexan-1-yl]amino}-6-{4-[(morpholin-4-yl)methyl]phenyl}pyrido[3,2-d]pyrimidine-8-carboxamide (4.7 mg, 89% enantiomeric purity) with a RT 3.579 min.

### Example 21: Synthesis of Compound 21 (S)-4-(azepan-3-ylamino)-6-(4-(morpholinomethyl)phenyl)pyrido[3,2-d]pyrimidine-8-carboxamide and (R)-4-(azepan-3-ylamino)-6-(4-(morpholinomethyl)phenyl)pyrido[3,2-d]pyrimidine-8-carboxamid

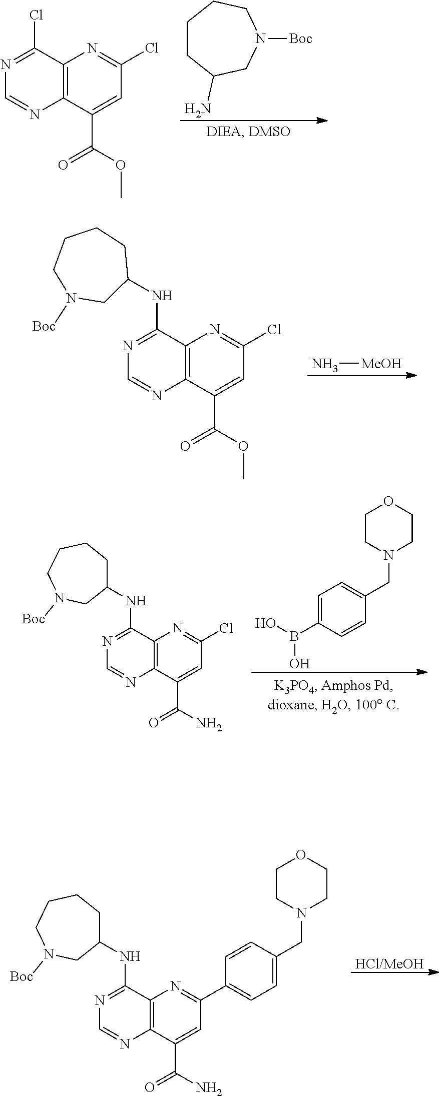

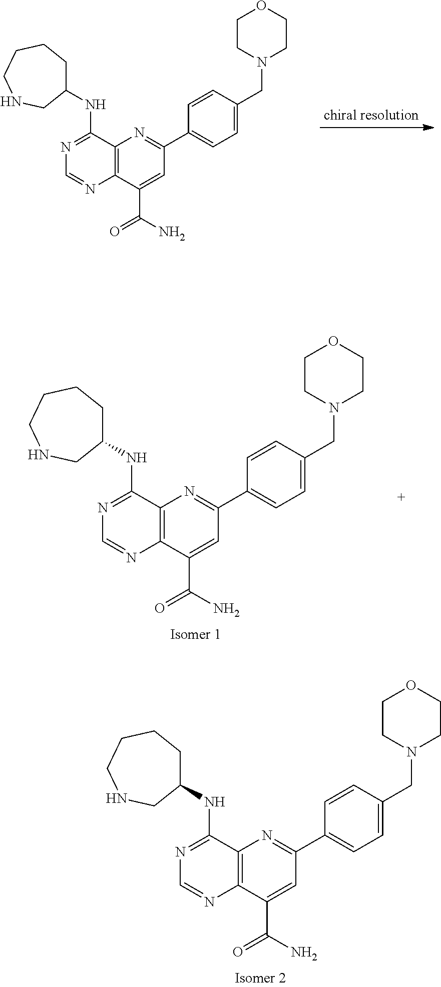 [SMILES: NC(=O)c1cc(-c2ccc(CN3CCOCC3)cc2)nc2c(NC3CCCCNC3)ncnc12 ; NC(=O)c1cc(-c2ccc(CN3CCOCC3)cc2)nc2c(N[C@H]3CCCCNC3)ncnc12 ; NC(=O)c1cc(-c2ccc(CN3CCOCC3)cc2)nc2c(N[C@@H]3CCCCNC3)ncnc12]

methyl 4-((1-(tert-butoxycarbonyl)azepan-3-yl)amino)-6-chloropyrido[3,2-d]pyrimidine-8-carboxylate: To a stirred mixture of methyl 4,6-dichloropyrido[3,2-d]pyrimidine-8-carboxylate (300.00 mg, 0.958 mmol) and tert-butyl 3-aminoazepane-1-carboxylate (172.88 mg, 0.766 mmol) in ACN (3 ml) was added DIEA (360.12 mg, 2.647 mmol). The resulting mixture was stirred for 4 h at 40° C. under nitrogen atmosphere. The resulting mixture was concentrated under vacuum. The residue was purified by silica gel column chromatography, eluted with PE/EtOAc (1:1) to afford tert-butyl 3-[[6-chloro-8-(methoxycarbonyl)pyrido[3,2-d]pyrimidin-4-yl]amino]azepane-1-carboxylate (140 mg, 33.53%) as a yellow solid.

tert-butyl 3-((8-carbamoyl-6-chloropyrido[3,2-d]pyrimidin-4-yl)amino)azepane-1-carboxylate: A mixture of tert-butyl 3-[[6-chloro-8-(methoxycarbonyl)pyrido[3,2-d]pyrimidin-4-yl]amino]azepane-1-carboxylate (130.00 mg, 0.298 mmol) in NH₃ (g) in MeOH (8 mL) was stirred for 2 h at 40° C. under nitrogen atmosphere. The resulting mixture was concentrated under vacuum. This resulted in tert-butyl 3-([8-carbamoyl-6-chloropyrido[3,2-d]pyrimidin-4-yl]amino)azepane-1-carboxylate (120 mg, 77.63%) as a yellow solid used in the next step directly.

tert-butyl3-((8-carbamoyl-6-(4-(morpholinomethyl)phenyl)pyrido[3,2-d]pyrimidin-4-yl)amino)azepane-1-carboxylate: To a solution of tert-butyl 3-[(8-carbamoyl-6-chloroquinazolin-4-yl)amino]azepane-1-carboxylate (110 mg, 0.213 mmol) and 4-[[4-(4,4,5,5-tetramethyl-1,3-dioxolan-2-yl)phenyl]methyl]morpholine (82.06 mg, 0.255 mmol) in 1,4-Dioxane (5 mL) and H₂O (1.00 mL) were added K₃PO₄ (142.58 mg, 0.638 mmol) and AMPHOS-PdCl₂ (15.85 mg, 0.021 mmol). The resulting mixture was stirred for 2 h at 100° C. under a nitrogen atmosphere, the resulting mixture was concentrated under reduced pressure. The residue was purified by silica gel column chromatography, eluted with CH₂Cl₂/MeOH (9:1) to afford tert-butyl 3-([8-carbamoyl-6-[4-(morpholin-4-ylmethyl)phenyl]pyrido[3,2-d]pyrimidin-4-yl]amino)azepane-1-carboxylate (180 mg, 97.70%) as a yellow solid.

4-(azepan-3-ylamino)-6-(4-(morpholinomethyl)phenyl)pyrido[3,2-d]pyrimidine-8-carboxamide: A solution of tert-butyl 3-([8-carbamoyl-6-[4-(morpholin-4-ylmethyl)phenyl]pyrido[3,2-d]pyrimidin-4-yl]amino)azepane-1-carboxylate (170 mg, 0.196 mmol) in HCl (g) in MeOH (8 mL) was stirred for 2 h at room temperature under nitrogen atmosphere. The resulting mixture was concentrated under vacuum. The crude product (140 mg) was purified by Prep-HPLC with the following conditions (2#SHIMADZU (HPLC-01)): Column, XBridge Shield RP18 OBD Column, 30*150 mm, 5 μm; mobile phase, Water (10 mmol/L NH₄HCO₃+0.1% [28% NH₃ in H₂O]) and ACN (20% PhaseB up to 40% in 8 min.); Detector, UV 254 nm. This resulted in 4-(azepan-3-ylamino)-6-[4-(morpholin-4-ylmethyl)phenyl]pyrido[3,2-d]pyrimidine-8-carboxamide (40 mg, 42.66%) as a light-yellow solid.

(S)-4-(azepan-3-ylamino)-6-(4-(morpholinomethyl)phenyl)pyrido[3,2-d]pyrimidine-8-carboxamide and (R)-4-(azepan-3-ylamino)-6-(4-(morpholinomethyl)phenyl)pyrido[3,2-d]pyrimidine-8-carboxamide: The product (40 mg) was chiral separated by Chiral-Prep-HPLC with the following conditions (Prep-HPLC-032): Column, CHIRALPAK ID, 2*25 cm (5 μm); mobile phase, MTBE (10 mM NH₃-MEOH) and EtOH (hold 50% EtOH in 14 min); Detector, UV 254 nm. This resulted in 4-[(3S)-azepan-3-ylamino]-6-[4-(morpholin-4-ylmethyl)phenyl]pyrido[3,2-d]pyrimidine-8-carboxamide (4.3 mg, 10.62%) as a yellow solid and 4-[(3R)-azepan-3-ylamino]-6-[4-(morpholin-4-ylmethyl)phenyl]pyrido[3,2-d]pyrimidine-8-carboxamide (3.6 mg, 9.04%) as a yellow solid.

Isomer 1: HPLC 96.8% purity, RT=2.351 min. MS:m/z=462.2 [M+H]⁺. ¹H NMR (300 MHz, DMSO-d6, ppm): δ 10.04 (d, J=3.8 Hz, 1H), 8.81 (s, 1H), 8.57-8.46 (m, 2H), 8.36-8.19 (m, 3H), 7.49 (d, J=7.9 Hz, 2H), 4.39 (s, 1H), 3.57 (dd, J=10.3, 5.7 Hz, 7H), 3.06-2.74 (m, 3H), 2.38 (t, J=4.7 Hz, 4H), 2.0-1.90 (m, 2H), 1.88-1.56 (m, 4H).

Isomer 2: HPLC 95.2% purity, RT=2.359 min. MS: m/z=462.2 [M+H]⁺. ¹H NMR (300 MHz, DMSO-d6, ppm): δ 10.04 (d, J=3.8 Hz, 1H), 8.81 (s, 1H), 8.57-8.46 (m, 2H), 8.36-8.19 (m, 3H), 7.49 (d, J=7.9 Hz, 2H), 4.39 (s, 1H), 3.57 (dd, J=10.3, 5.7 Hz, 7H), 3.06-2.74 (m, 3H), 2.38 (t, J=4.7 Hz, 4H), 2.0-1.90 (m, 2H), 1.88-1.56 (m, 4H).

### Example 22: Synthesis of Compound 22 4-((3R,4R)-3-amino-4-fluoropiperidin-1-yl)-6-(4-(morpholinomethyl)phenyl)pyrido[3,2-d]pyrimidine-8-carboxamide, 4-((3R,4S)-3-amino-4-fluoropiperidin-1-yl)-6-(4-(morpholinomethyl)phenyl)pyrido[3,2-d]pyrimidine-8-carboxamide, 4-((3S,4S)-3-amino-4-fluoropiperidin-1-yl)-6-(4-(morpholinomethyl)phenyl)pyrido[3,2-d]pyrimidine-8-carboxamide and 4-((3S,4R)-3-amino-4-fluoropiperidin-1-yl)-6-(4-(morpholinomethyl)phenyl)pyrido[3,2-d]pyrimidine-8-carboxamide

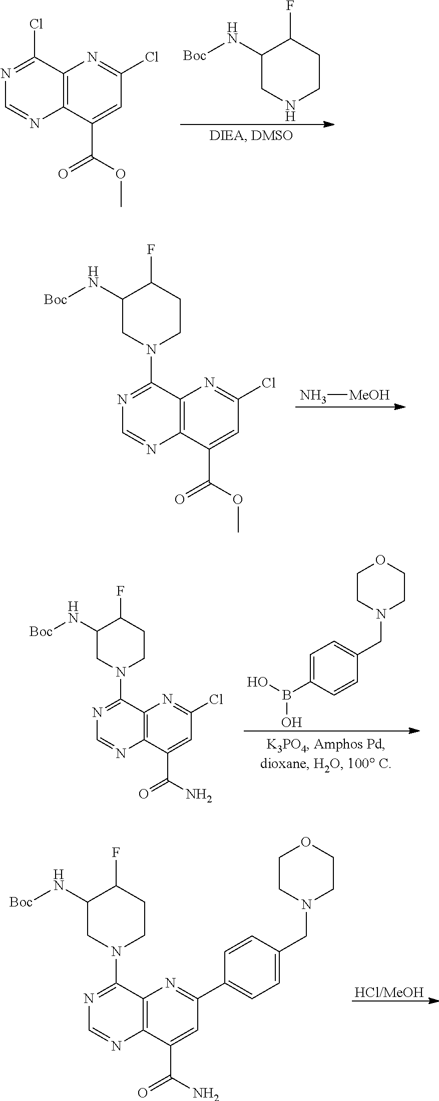

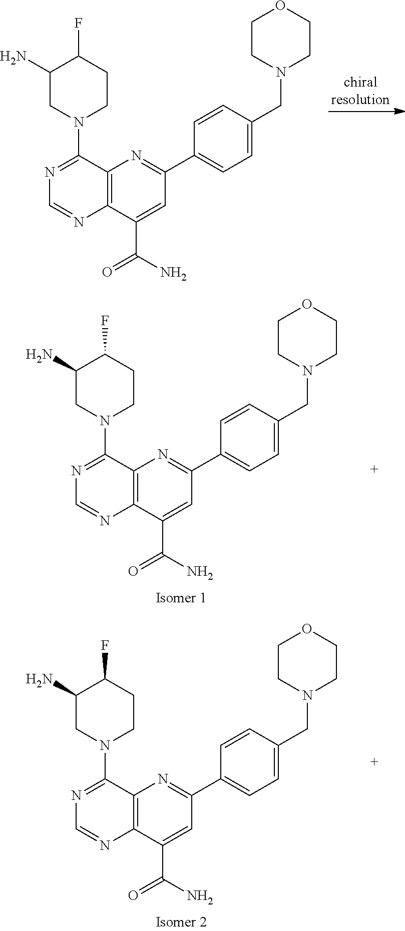 [SMILES: NC(=O)c1cc(-c2ccc(CN3CCOCC3)cc2)nc2c(N3CCC(F)C(N)C3)ncnc12 ; NC(=O)c1cc(-c2ccc(CN3CCOCC3)cc2)nc2c(N3CC[C@@H](F)[C@H](N)C3)ncnc12 ; NC(=O)c1cc(-c2ccc(CN3CCOCC3)cc2)nc2c(N3CC[C@H](F)[C@H](N)C3)ncnc12]

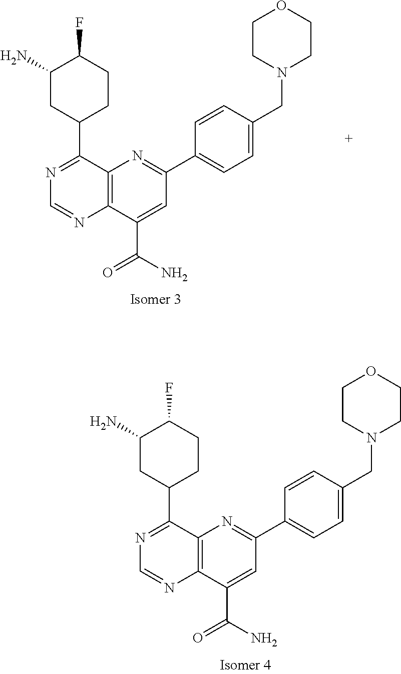 [SMILES: NC(=O)c1cc(-c2ccc(CN3CCOCC3)cc2)nc2c(C3CC[C@H](F)[C@@H](N)C3)ncnc12 ; NC(=O)c1cc(-c2ccc(CN3CCOCC3)cc2)nc2c(C3CC[C@@H](F)[C@@H](N)C3)ncnc12]

methyl4-(3-((tert-butoxycarbonyl)amino)-4-fluoropiperidin-1-yl)-6-chloropyrido[3,2-d]pyrimidine-8-carboxylate: To a stirred mixture of methyl 4,6-dichloropyrido[3,2-d]pyrimidine-8-carboxylate (580 mg, 1.33 mmol) and tert-butyl N-(4-fluoropiperidin-3-yl)carbamate (250 mg, 1.06 mmol) in ACN (10 mL) were added DIEA (544 mg, 4 mmol). The resulting mixture was stirred for 4 h at 40° C. under nitrogen atmosphere. The resulting mixture was concentrated under vacuum. The residue was purified by silica gel column chromatography, eluted with PE/EtOAc (3:2) to afford methyl 4-[3-[(tert-butoxycarbonyl)amino]-4-fluoropiperidin-1-yl]-6-chloropyrido[3,2-d]pyrimidine-8-carboxylate (500 mg, 85.29%) as a yellow solid.

tert-butyl(1-(8-carbamoyl-6-chloropyrido[3,2-d]pyrimidin-4-yl)-4-fluoropiperidin-3-yl)carbamate: A mixture of methyl 4-[3-[(tert-butoxycarbonyl)amino]-4-fluoropiperidin-1-yl]-6-chloropyrido[3,2-d]pyrimidine-8-carboxylate (500 mg, 1.18 mmol) in NH₃ (g) in MeOH (10 mL) was stirred for 2 h at 40° C. under nitrogen atmosphere. The resulting mixture was concentrated under vacuum. This resulted in tert-butyl N-(1-[8-carbamoyl-6-chloropyrido[3,2-d]pyrimidin-4-yl]-4-fluoropiperidin-3-yl)carbamate (460 mg, 99.81%) as a light-yellow solid.

tert-butyl(1-(8-carbamoyl-6-(4-(morpholinomethyl)phenyl)pyrido[3,2-d]pyrimidin-4-yl)-4-fluoropiperidin-3-yl)carbamate: To a mixture of tert-butyl N-(1-[8-carbamoyl-6-chloropyrido[3,2-d]pyrimidin-4-yl]-4-fluoropiperidin-3-yl)carbamate (460 mg, 1 mmol) and 4-[[4-(4,4,5,5-tetramethyl-1,3,2-dioxaborolan-2-yl)phenyl]methyl]morpholine (400 mg, 0.626 mmol) in 1,4-Dioxane (10 mL) and H₂O (2 mL) were added AMPHOS-PdCl₂ (78 mg, 0.1 mmol) and K₃PO₄ (690 mg, 3.2 mmol). After stirring for 2 h at 100° C. under a nitrogen atmosphere, the resulting mixture was concentrated under reduced pressure. The residue was purified by silica gel column chromatography, eluted with CH₂Cl₂/MeOH (9:1) to afford tert-butyl N-(1-[8-carbamoyl-6-[4-(morpholin-4-ylmethyl)phenyl]pyrido[3,2-d]pyrimidin-4-yl]-4-fluoropiperidin-3-yl)carbamate (290 mg, 47%) as a yellow solid.

4-(3-amino-4-fluoropiperidin-1-yl)-6-(4-(morpholinomethyl)phenyl)pyrido[3,2-d]pyrimidine-8-carboxamide: A solution of tert-butyl N-(1-[8-carbamoyl-6-[4-(morpholin-4-ylmethyl)phenyl]pyrido[3,2-d]pyrimidin-4-yl]-4-fluoropiperidin-3-yl)carbamate (290.00 mg, 0.431 mmol) in HCl (g) in MeOH (8 mL) was stirred for 1 h at room temperature under nitrogen atmosphere. The resulting mixture was concentrated under vacuum. The crude product (250 mg) was purified by Prep-HPLC with the following conditions (2#SHIMADZU (HPLC-01)): Column, XBridge Prep OBD C18 Column, 30ᵢÀ150 mm 5 um; mobile phase, Water (10 mmol/L NH₄HCO₃+0.1% [28% NH₃ in H₂O]) and ACN (15% Phase B up to 40% in 8 min). This resulted in 4-(3-amino-4-fluoropiperidin-1-yl)-6-[4-(morpholin-4-ylmethyl)phenyl]pyrido[3,2-d]pyrimidine-8-carboxamide (170 mg, 77.41%) as a yellow green solid.

4-((3R,4R)-3-amino-4-fluoropiperidin-1-yl)-6-(4-(morpholinomethyl)phenyl)pyrido[3,2-d]pyrimidine-8-carboxamide, 4-((3R,4S)-3-amino-4-fluoropiperidin-1-yl)-6-(4-(morpholinomethyl)phenyl)pyrido[3,2-d]pyrimidine-8-carboxamide, 4-((3S,4S)-3-amino-4-fluoropiperidin-1-yl)-6-(4-(morpholinomethyl)phenyl)pyrido[3,2-d]pyrimidine-8-carboxamide and 4-((3S,4R)-3-amino-4-fluoropiperidin-1-yl)-6-(4-(morpholinomethyl)phenyl)pyrido[3,2-d]pyrimidine-8-carboxamide: The product (170 mg) was chiral separated by Chiral-Prep-HPLC with the following conditions (Prep-HPLC-083): Column, CHIRALPAK IG, 3*25 cm, 5 um; mobile phase, MTBE (10 mM NH₃-MEOH)— and MeOH— (hold 50% MeOH— in 18 min); Detector, UV 254 nm. This resulted in 4-[(3S,4S)-3-amino-4-fluoropiperidin-1-yl]-6-[4-(morpholin-4-ylmethyl)phenyl]pyrido[3,2-d]pyrimidine-8-carboxamide (43.7 mg, 27.98%) as a yellow solid, 4-[(3S,4S)-3-amino-4-fluoropiperidin-1-yl]-6-[4-(morpholin-4-ylmethyl)phenyl]pyrido[3,2-d]pyrimidine-8-carboxamide (43.7 mg, 27.98%) as a yellow solid, 4-[(3S,4R)-3-amino-4-fluoropiperidin-1-yl]-6-[4-(morpholin-4-ylmethyl)phenyl]pyrido[3,2-d]pyrimidine-8-carboxamide (22.7 mg, 14.39%) as a light yellow solid and 4-[(3R,4S)-3-amino-4-fluoropiperidin-1-yl]-6-[4-(morpholin-4-ylmethyl)phenyl]pyrido[3,2-d]pyrimidine-8-carboxamide (21.1 mg, 13.44%) as a light yellow solid.

Isomer 1: HPLC 97.6% purity, RT=2.13 min. MS: m/z=462.2 [M+H]⁺.

¹H NMR (300 MHz, DMSO-d6, ppm): δ 10.02 (d, J=3.7 Hz, 1H), 8.80 (s, 1H), 8.56 (s, 1H), 8.20 (d, J=3.7 Hz, 1H), 8.13 (d, J=8.0 Hz, 2H), 7.49 (d, J=8.1 Hz, 2H), 5.54-5.01 (m, 1H), 4.96-4.80 (m, 1H), 3.68 (s, 1H), 3.64-3.51 (m, 6H), 3.33 (s, 2H), 3.12-3.02 (m, 1H), 2.39 (t, J=4.7 Hz, 4H), 2.18 (q, J=9.6, 8.3 Hz, 1H), 2.18-1.87 (m, 3H).

Isomer 2: HPLC 99.5% purity, RT=2.13 min. MS: m/z=462.2 [M+H]⁺. ¹H NMR (300 MHz, DMSO-d6, ppm): δ 10.02 (d, J=3.7 Hz, 1H), 8.80 (s, 1H), 8.56 (s, 1H), 8.20 (d, J=3.7 Hz, 1H), 8.13 (d, J=8.0 Hz, 2H), 7.49 (d, J=8.1 Hz, 2H), 5.54-5.01 (m, 1H), 4.96-4.80 (m, 1H), 3.68 (s, 1H), 3.64-3.51 (m, 6H), 3.33 (s, 2H), 3.12-3.02 (m, 1H), 2.39 (t, J=4.7 Hz, 4H), 2.18 (q, J=9.6, 8.3 Hz, 1H), 2.18-1.87 (m, 3H).

Isomer 3: HPLC 98.5% purity, RT=2.07 min. MS: m/z=462.2 [M+H]⁺. ¹H NMR (300 MHz, DMSO-d6, ppm): δ 10.02 (d, J=3.7 Hz, 1H), 8.80 (s, 1H), 8.56 (s, 1H), 8.20 (d, J=3.7 Hz, 1H), 8.13 (d, J=8.0 Hz, 2H), 7.49 (d, J=8.1 Hz, 2H), 5.54-5.01 (m, 1H), 4.96-4.80 (m, 1H), 3.68 (s, 1H), 3.64-3.51 (m, 6H), 3.33 (s, 2H), 3.12-3.02 (m, 1H), 2.39 (t, J=4.7 Hz, 4H), 2.18 (q, J=9.6, 8.3 Hz, 1H), 2.18-1.87 (m, 3H).

Isomer 4: HPLC 99.0% purity, RT=2.07 min. MS: m/z=462.2 [M+H]⁺. ¹H NMR (300 MHz, DMSO-d6, ppm): δ 10.02 (d, J=3.7 Hz, 1H), 8.80 (s, 1H), 8.56 (s, 1H), 8.20 (d, J=3.7 Hz, 1H), 8.13 (d, J=8.0 Hz, 2H), 7.49 (d, J=8.1 Hz, 2H), 5.54-5.01 (m, 1H), 4.96-4.80 (m, 1H), 3.68 (s, 1H), 3.64-3.51 (m, 6H), 3.33 (s, 2H), 3.12-3.02 (m, 1H), 2.39 (t, J=4.7 Hz, 4H), 2.18 (q, J=9.6, 8.3 Hz, 1H), 2.18-1.87 (m, 3H).

### Example 23: Synthesis of Compound 23 4-((2-aminoethyl)amino)-6-(4-(morpholinomethyl)phenyl)pyrido[3,2-d]pyrimidine-8-carboxamide

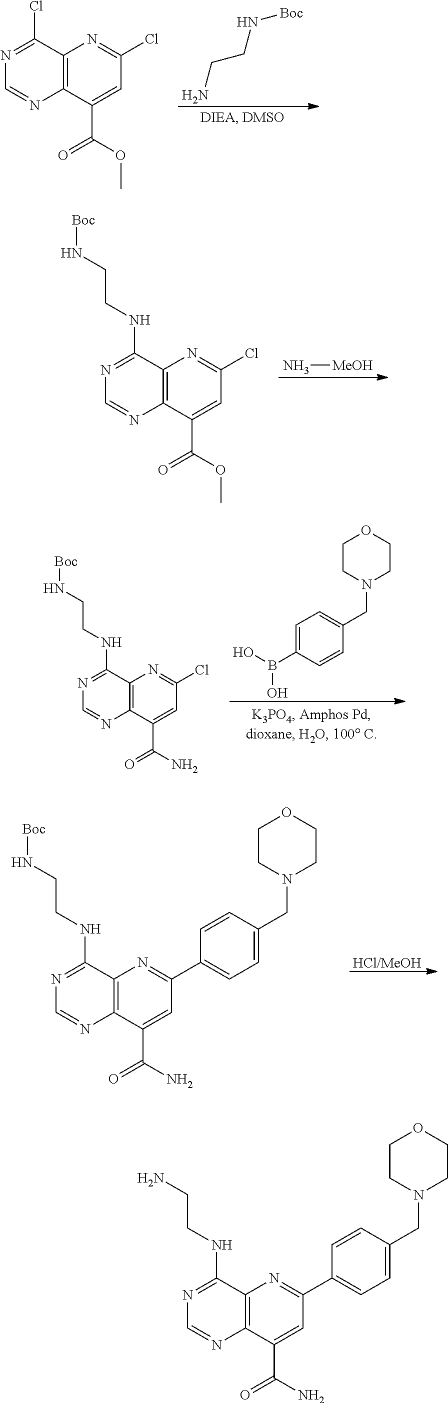

Methyl 4-((2-((tert-butoxycarbonyl)amino)ethyl)amino)-6-chloropyrido[3,2-d]pyrimidine-8-carboxylate: To a stirred mixture of methyl 4,6-dichloropyrido[3,2-d]pyrimidine-8-carboxylate (200 mg, 0.369 mmol) and tert-butyl N-(2-aminoethyl)carbamate (49.77 mg, 0.295 mmol) in ACN (10 mL) were added DIEA (150.56 mg, 1.107 mmol). The resulting mixture was stirred for 2 h at 50° C. under nitrogen atmosphere. The resulting mixture was concentrated under vacuum. The residue was purified by silica gel column chromatography, eluted with PE/EtOAc (3:7) to afford methyl 4-([2-[(tert-butoxycarbonyl)amino]ethyl]amino)-6-chloropyrido[3,2-d]pyrimidine-8-carboxylate (140 mg, 54.67%) as a light-yellow solid.

tert-butyl(2-((8-carbamoyl-6-chloropyrido[3,2-d]pyrimidin-4-yl)amino)ethyl)carbamate: A mixture of methyl 4-([2-[(tert-butoxycarbonyl)amino]ethyl]amino)-6-chloropyrido[3,2-d]pyrimidine-8-carboxylate (140 mg, 0.202 mmol) in NH₃ (g) in MeOH (5 mL) was stirred for 2 h at 40° C. under nitrogen atmosphere. The resulting mixture was concentrated under vacuum. This resulted in tert-butyl N-[2-([8-carbamoyl-6-chloropyrido[3,2-d]pyrimidin-4-yl]amino)ethyl]carbamate (93 mg, 98.31%) as a light-yellow solid.

tert-butyl(2-((8-carbamoyl-6-(4-(morpholinomethyl)phenyl)pyrido[3,2-d]pyrimidin-4-yl)amino)ethyl)carbamate: To a mixture of tert-butyl N-[2-([8-carbamoyl-6-chloropyrido[3,2-d]pyrimidin-4-yl]amino)ethyl]carbamate (93 mg, 0.198 mmol) and 4-[[4-(4,4,5,5-tetramethyl-1,3,2-dioxaborolan-2-yl)phenyl]methyl]morpholine (75.94 mg, 0.238 mmol) in 1,4-Dioxane (5 mL) and H₂O (1 mL) were added K₃PO₄ (132.90 mg, 0.595 mmol) and AMPHOS-PdCl₂ (14.78 mg, 0.020 mmol). The resulting mixture was stirred for 2 h at 100° C. under a nitrogen atmosphere, the resulting mixture was concentrated under reduced pressure. The residue was purified by silica gel column chromatography, eluted with CH₂Cl₂/MeOH (9:1) to afford tert-butyl N-[2-([8-carbamoyl-6-[4-(morpholin-4-ylmethyl)phenyl]pyrido[3,2-d]pyrimidin-4-yl]amino)ethyl]carbamate (80 mg, 76.07%) as a light-yellow solid.

4-((2-aminoethyl)amino)-6-(4-(morpholinomethyl)phenyl)pyrido[3,2-d]pyrimidine-8-carboxamide: A solution of tert-butyl N-[2-([8-carbamoyl-6-[4-(morpholin-4-ylmethyl)phenyl]pyrido[3,2-d]pyrimidin-4-yl]amino)ethyl]carbamate (80 mg, 0.151 mmol) in HCl (g) in MeOH (5 mL) was stirred for 1 h at room temperature under nitrogen atmosphere. The resulting mixture was concentrated under vacuum. The crude product (60 mg) was purified by Prep-HPLC with the following conditions (2#SHIMADZU (HPLC-01)): Column, XBridge Prep OBD C18 Column, 30×150 mm, 5 μum; mobile phase, water (10 mmol/L NH₄HCO₃) and ACN (10% PhaseB up to 30% in 8 min.); Detector, UV 254 nm. This resulted in 4-[(2-aminoethyl)amino]-6-[4-(morpholin-4-ylmethyl)phenyl]pyrido[3,2-d]pyrimidine-8-carboxamide (26.3 mg, 41.94%) as a light-yellow solid.

HPLC 98.0% purity, RT=1.923 min. MS: m/z=408.3 [M+H]⁺.

¹H NMR (300 MHz, DMSO-d6, ppm): δ 10.08 (s, 1H), 8.83 (s, 1H), 8.54 (s, 1H), 8.35 (t, J=7.2 Hz, 2H), 8.24 (s, 1H), 7.48 (dd, J=8.3, 3.3 Hz, 2H), 3.64-3.62 (m, 6H), 3.51 (s, 2H), 3.43-3.28 (m, 1H), 2.87 (t, J=6.6 Hz, 1H), 2.39 (t, J=4.6 Hz, 4H).

### Example 24: Synthesis of Compound 24 (S)-6-(4-(2-methoxyethoxy) phenyl)-4-(piperidin-3-ylamino) pyrido[3,2-d]pyrimidine-8-carboxamide

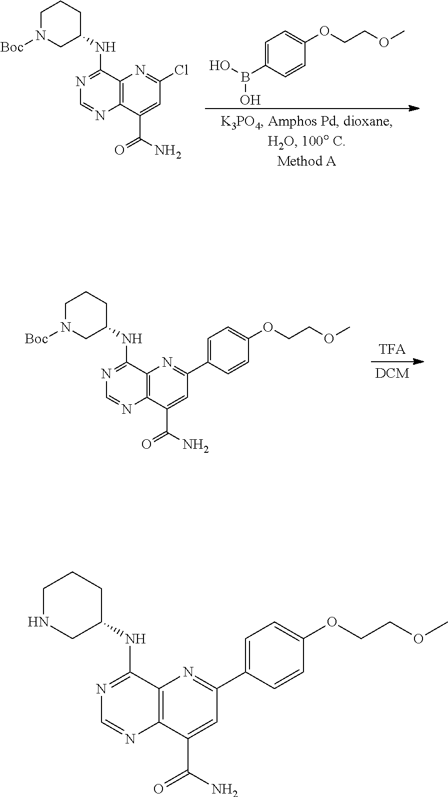 [SMILES: CC(C)(C)OC(=O)N1CCC[C@H](Nc2ncnc3c(C(N)=O)cc(Cl)nc23)C1 ; COCCOc1ccc(B(O)O)cc1 ; COCCOc1ccc(-c2cc(C(N)=O)c3ncnc(N[C@H]4CCCN(C(=O)OC(C)(C)C)C4)c3n2)cc1 ; COCCOc1ccc(-c2cc(C(N)=O)c3ncnc(N[C@H]4CCCNC4)c3n2)cc1]

Tert-butyl(S)-3-((8-carbamoyl-6-(4-(2-methoxyethoxy) phenyl) pyrido[3,2-d]pyrimidin-4-yl) amino) piperidine-1-carboxylate: To a solution of tert-butyl (3S)-3-([8-carbamoyl-6-chloropyrido[3,2-d]pyrimidin-4-yl]amino)piperidine-1-carboxylate (300 mg, 0.666 mmol) and 4-(2-methoxyethoxy) phenyl boronic acid (431 mg, 2.089 mmol,) in 1,4-Dioxane (15.00 mL) and H₂O (3.00 mL) were added K₃PO₄ (654 mg, 3.084 mmol) and AMPHOS-PdCl₂ (89 mg, 0.119 mmol). The resulting mixture was stirred for 2 h at 100° C. under a nitrogen atmosphere. The resulting mixture was concentrated under reduced pressure. The residue was purified by silica gel column chromatography, eluted with PE/EtOAc (1:1) to afford tert-butyl (3S)-3-([8-carbamoyl-6-[4-(2-methoxyethoxy) phenyl]pyrido[3,2-d]pyrimidin-4-yl]amino) piperidine-1-carboxylate (200 mg, 71.27%) as a yellow solid.

(S)-6-(4-(2-methoxyethoxy) phenyl)-4-(piperidin-3-ylamino) pyrido [3,2-d]pyrimidine-8-carboxamide: To a stirred solution of tert-butyl (3S)-3-([8-carbamoyl-6-[4-(2-methoxyethoxy) phenyl]pyrido[3,2-d]pyrimidin-4-yl]amino) piperidine-1-carboxylate (180 mg, 0.342 mmol) in DCM (8.00 mL) was added TFA (2.00 mL) at room temperature under nitrogen atmosphere. The resulting mixture was stirred for 2 h at room temperature under nitrogen atmosphere. The resulting mixture was concentrated under reduced pressure. The crude product was purified by Prep-HPLC with the following conditions (2#SHIMADZU (HPLC-01)): Column, XBridge Prep OBD C18 Column, 30×150 mm, 5 μm; mobile phase, water (10 mmol/L NH₄HCO₃) and ACN (18% PhaseB up to 48% in 8 min.); Detector, UV 254 nm. (S)-6-(4-(2-methoxyethoxy) phenyl)-4-(piperidin-3-ylamino) pyrido [3,2-d]pyrimidine-8-carboxamide (112.7 mg, 76.20%) product was obtained as light-yellow solid.

HPLC: 99.5% purity, RT=8.98 min. MS: m/z=423.1[M+H]⁺. ¹H NMR (300 MHz, DMSO-d6) δ 10.04 (d, J=3.4 Hz, 1H), 8.78 (s, 1H), 8.53 (s, 1H), 8.31 (dd, J=8.7, 3.2 Hz, 3H), 8.20 (d, J=3.7 Hz, 1H), 7.10 (d, J=8.4 Hz, 2H), 4.51-4.05 (m, 3H), 3.74-3.65 (m, 2H), 3.33 (s, 2H), 3.04 (d, J=11.7 Hz, 1H), 2.87-2.67 (m, 2H), 2.58 (d, J=10.6 Hz, 2H), 2.01-1.31 (m, 4H).

### Example 25: Synthesis of Compound 25 (R)-4-((2-aminopropyl)amino)-6-(4-(morpholinomethyl)phenyl)pyrido[3,2-d]pyrimidine-8-carboxamide and (S)-4-((2-aminopropyl)amino)-6-(4-(morpholinomethyl)phenyl)pyrido[3,2-d]pyrimidine-8-carboxamide

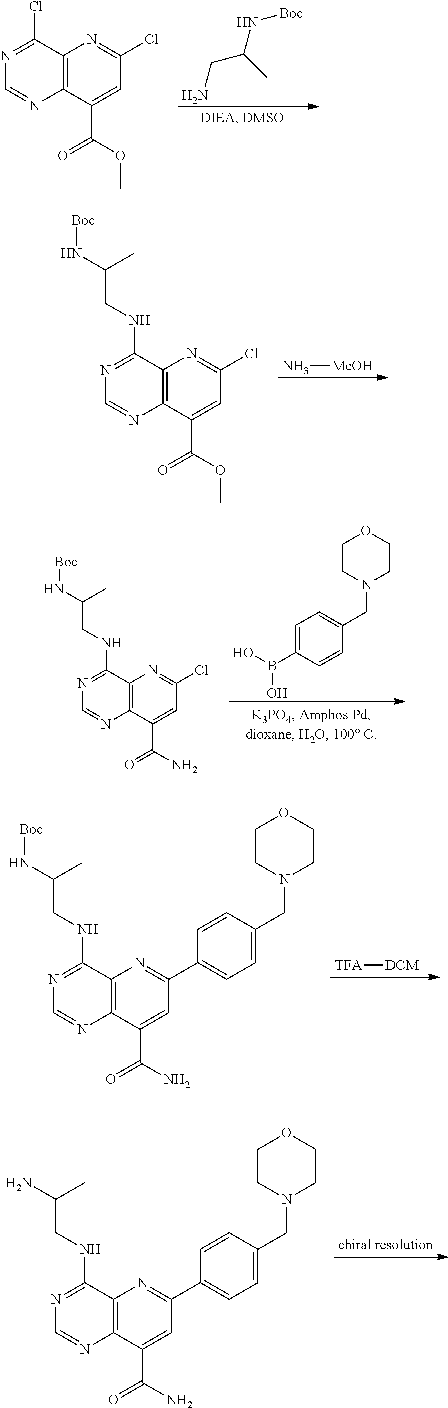

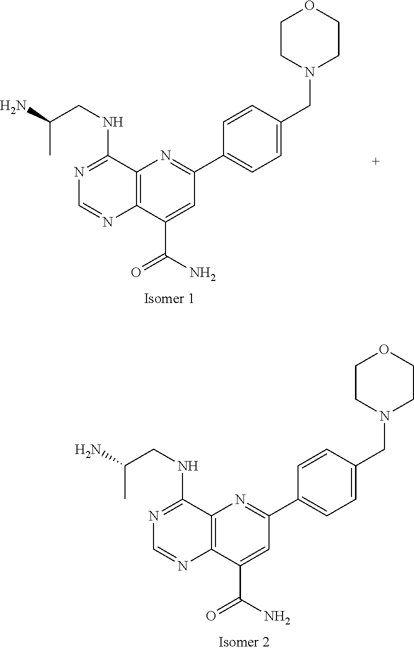 [SMILES: C[C@@H](N)CNc1ncnc2c(C(N)=O)cc(-c3ccc(CN4CCOCC4)cc3)nc12 ; C[C@H](N)CNc1ncnc2c(C(N)=O)cc(-c3ccc(CN4CCOCC4)cc3)nc12]

methyl 4-((2-((tert-butoxycarbonyl)amino)propyl)amino)-6-chloropyrido[3,2-d]pyrimidine-8-carboxylate: A solution of methyl 4,6-dichloropyrido[3,2-d]pyrimidine-8-carboxylate (400 mg, 0.891 mmol) and tert-butyl N-(1-aminopropan-2-yl)carbamate (539.46 mg, 2.941 mmol), DIEA (606.26 mg, 4.456 mmol) in ACN (10 mL) was stirred for 16 h at 40° C. under nitrogen atmosphere. The resulting mixture was concentrated under vacuum. This resulted in methyl 4-([2-[(tert-butoxycarbonyl)amino]propyl]amino)-6-chloropyrido[3,2-d]pyrimidine-8-carboxylate (150 mg, 23.26%) as a yellow solid.

tert-butyl (1-((8-carbamoyl-6-chloropyrido[3,2-d]pyrimidin-4-yl)amino)propan-2-yl)carbamate: A solution of methyl 4-([2-[(tert-butoxycarbonyl)amino]propyl]amino)-6-chloropyrido[3,2-d]pyrimidine-8-carboxylate (140 mg, 0.193 mmol) in NH₃ (g) in MeOH (3 mL) was stirred for 4 h at 40° C. under nitrogen atmosphere. The resulting mixture was concentrated under vacuum. This resulted in tert-butyl N-[1-([8-carbamoyl-6-chloropyrido[3,2-d]pyrimidin-4-yl]amino)propan-2-yl]carbamate (120 mg, 94.51%) as a yellow solid.

tert-butyl(1-((8-carbamoyl-6-(4-(morpholinomethyl)phenyl)pyrido[3,2-d]pyrimidin-4-yl)amino)propan-2-yl)carbamate: A solution of tert-butyl N-[1-([8-carbamoyl-6-chloropyrido[3,2-d]pyrimidin-4-yl]amino)propan-2-yl]carbamate (120 mg, 0.199 mmol) and 4-[[4-(4,4,5,5-tetramethyl-1,3,2-dioxaborolan-2-yl)phenyl]methyl]morpholine (115.23 mg, 0.361 mmol), AMPHOS-PdCl₂ (22.30 mg, 0.030 mmol), K₃PO₄ (200.10 mg, 0.896 mmol) in 1,4-Dioxane (10 mL), H₂O (3 mL) was stirred for 2 h at 100° C. under nitrogen atmosphere. The resulting mixture was concentrated under vacuum. The residue was purified by silica gel column chromatography, eluted with PE/EtOAc (1:1) to afford tert-butyl N-[1-([8-carbamoyl-6-[4-(morpholin-4-ylmethyl)phenyl]pyrido[3,2-d]pyrimidin-4-yl]amino)propan-2-yl]carbamate (100 mg, 67.95%) as a yellow solid.

4-((2-aminopropyl)amino)-6-(4-(morpholinomethyl)phenyl)pyrido[3,2-d]pyrimidine-8-carboxamide: A solution of tert-butyl N-[(2R)-1-([8-carbamoyl-6-[4-(morpholin-4-ylmethyl)phenyl]pyrido[3,2-d]pyrimidin-4-yl]amino)propan-2-yl]carbamate (100 mg) in HCl (gas) in 1,4-Dioxane (6 mL) was stirred for 2 h at 25° C. under nitrogen atmosphere. The solvent was removed, and the residue was purified by silica gel column chromatography, eluted with PE/EtOAc (8:1) to afford 4-[(2-aminopropyl)amino]-6-[4-(morpholin-4-ylmethyl)phenyl]pyrido[3,2-d]pyrimidine-8-carboxamide (50 mg, 97.18%) as an off-white solid.

(R)-4-((2-aminopropyl)amino)-6-(4-(morpholinomethyl)phenyl)pyrido[3,2-d]pyrimidine-8-carboxamide and (S)-4-((2-aminopropyl)amino)-6-(4-(morpholinomethyl)phenyl)pyrido[3,2-d]pyrimidine-8-carboxamide: 4-[(2-aminopropyl)amino]-6-[4-(morpholin-4-ylmethyl)phenyl]pyrido[3,2-d]pyrimidine-8-carboxamide (50 mg) was purified by Prep-HPLC with the following conditions (2#SHIMADZU (HPLC-01)): Column, XBridge Prep OBD C18 Column, 30×150 mm, 5 μm; mobile phase, water (10 mmol/L NH₄HCO₃+0.1% [28% NH₃ in H₂O]) and ACN (28% PhaseB up to 58% in 8 min.); Detector, UV 254 nm. The product was chiral separated by Chiral-Prep-HPLC with the following conditions (Agela High-pressure Flash): Column, CHIRALPAK IC, 3*25 cm, 5 μm; mobile phase, MTBE (10 mM NH₃-MEOH) and EtOH (hold 10% EtOH in 20 min.); Detector, UV 254 nm. This resulted in 4-[[(2R)-2-aminopropyl]amino]-6-[4-(morpholin-4-ylmethyl)phenyl]pyrido[3,2-d]pyrimidine-8-carboxamide (10.1 mg, 19.31%) as an off white solid and 4-[[(2S)-2-aminopropyl]amino]-6-[4-(morpholin-4-ylmethyl)phenyl]pyrido[3,2-d]pyrimidine-8-carboxamide (10.8 mg, 21.07%) as an off-white solid.

Isomer 1: HPLC 94.8% purity, RT=2.472 min. MS: m/z=422.2 [M+H]⁺.

¹H NMR (300 MHz, DMSO-d6, ppm): δ 10.08 (d, 1H), 8.86 (d, 2H), 8.56 (s, 1H), 8.45-8.30 (m, 2H), 8.25 (d, 1H), 7.50 (m, 2H), 3.59 (m, 8H), 3.54-3.42 (m, 1H), 2.40 (t, 4H), 1.09 (d, 3H).

Isomer 2: HPLC 95.8% purity, RT=2.472 min. MS: m/z=422.2 [M+H]⁺.

¹H NMR (300 MHz, DMSO-d6, ppm): δ 10.08 (d, 1H), 8.86 (d, 2H), 8.56 (s, 1H), 8.45-8.30 (m, 2H), 8.25 (d, 1H), 7.50 (m, 2H), 3.59 (m, 8H), 3.54-3.42 (m, 1H), 2.40 (t, 4H), 1.09 (d, 3H).

### Example 26: Synthesis of Compound 26 4-[[(3S,5S)-5-fluoropiperidin-3-yl]amino]-6-[4-(morpholin-4-ylmethyl) phenyl]pyrido[3,2-d]pyrimidine-8-carboxamide

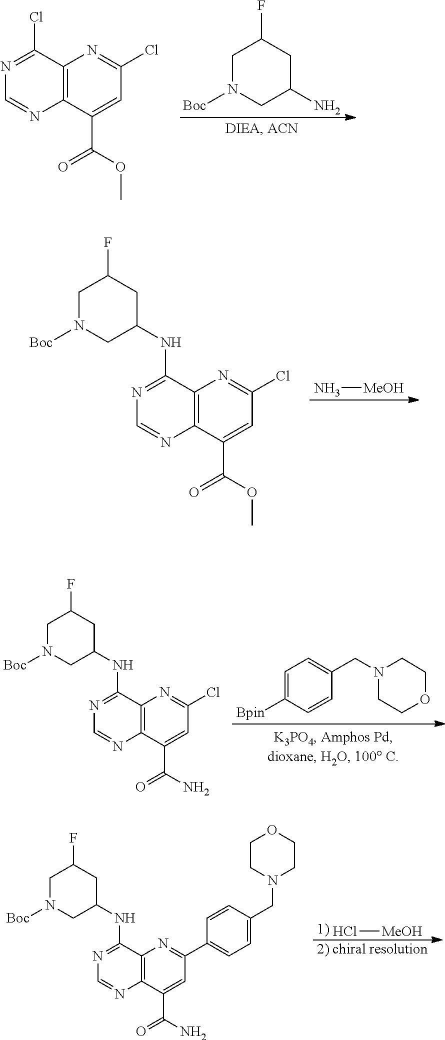

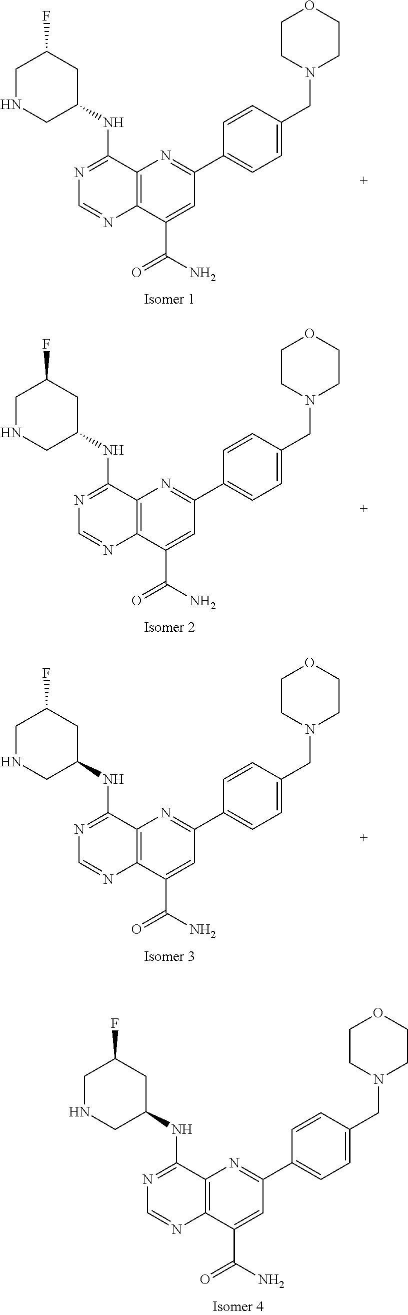 [SMILES: NC(=O)c1cc(-c2ccc(CN3CCOCC3)cc2)nc2c(N[C@@H]3CNC[C@H](F)C3)ncnc12 ; NC(=O)c1cc(-c2ccc(CN3CCOCC3)cc2)nc2c(N[C@@H]3CNC[C@@H](F)C3)ncnc12 ; NC(=O)c1cc(-c2ccc(CN3CCOCC3)cc2)nc2c(N[C@H]3CNC[C@H](F)C3)ncnc12 ; NC(=O)c1cc(-c2ccc(CN3CCOCC3)cc2)nc2c(N[C@H]3CNC[C@@H](F)C3)ncnc12]

tert-butyl 3-[[6-chloro-8-(methoxycarbonyl) pyrido[3,2-d]pyrimidin-4-yl]amino]-5-fluoropiperidine-1-carboxylate: To a stirred solution of methyl 4,6-dichloropyrido[3,2-d]pyrimidine-8-carboxylate (600.00 mg, 0.997 mmol) and tert-butyl 3-amino-5-fluoropiperidine-1carboxylate (270.00 mg, 1.175 mmol) in CH₃CN (10.00 mL, 243.588 mmol) was added DIEA (240.00 μL, 1764.115 mmol) at room temperature. The resulting mixture was stirred for 4 h at 60° C. The resulting mixture was concentrated under vacuum. The residue was purified by silica gel column chromatography, eluted with PE/EtOAc (1:1) to afford tert-butyl 3-[[6-chloro-8-(methoxycarbonyl) pyrido[3,2-d]pyrimidin-4-yl]amino]-5-fluoropiperidine-1-carboxylate (500 mg, 80.80%) as a brown solid.

tert-butyl 3-([8-carbamoyl-6-chloropyrido[3,2-d]pyrimidin-4-yl]amino)-5-fluoropiperidine-1-carboxylate: A solution of tert-butyl 3-[[6-chloro-8-(methoxycarbonyl) pyrido[3,2-d]pyrimidin-4-yl]amino]-5-fluoropiperidine-1-carboxylate (450.00 mg, 0.725 mmol) in NH₃ (g) in MeOH (10.00 mL, 45.800 mmol) was stirred for 1 h at room temperature. The resulting mixture was concentrated under vacuum to afford tert-butyl 3-([8-carbamoyl-6-chloropyrido[3,2-d]pyrimidin-4-yl]amino)-5-fluoropiperidine-1-carboxylate (300 mg, 97.35%) as a yellow solid.

tert-butyl 3-([8-carbamoyl-6-[4-(morpholin-4-ylmethyl)phenyl]pyrido[3,2-d]pyrimidin-4-yl]amino)-5-fluoropiperidine-1-carboxylate: To a solution of tert-butyl 3-([8-carbamoyl-6-chloropyrido[3,2-d]pyrimidin-4-yl]amino)-5-fluoropiperidine-1-carboxylate (280.00 mg, 0.659 mmol) and 4-[[4-(4,4,5,5-tetramethyl-1,3,2-dioxaborolan-2-yl)phenyl]methyl]morpholine (300.00 mg, 0.940 mmol) in 1,4-Dioxane (9.00 mL, 106.237 mmol) and H₂O (1.71 mL, 94.919 mmol) were added K₃PO₄ (420.00 mg, 1.880 mmol) and AMPHOS-PdCl₂ (48.00 mg, 0.064 mmol). After stirring for 2 h at 100° C. under a nitrogen atmosphere, the resulting mixture was concentrated under reduced pressure. The residue was purified by silica gel column chromatography, eluted with DCM/MeOH (10:1) to afford tert-butyl 3-([8-carbamoyl-6-[4-(morpholin-4-ylmethyl) phenyl]pyrido[3,2-d]pyrimidin-4-yl]amino)-5-fluoropiperidine-1-carboxylate (310 mg, 38.25%) as a brown solid.

4-[[(3S,5R)-5-fluoropiperidin-3-yl]amino]-6-[4-(morpholin-4-ylmethyl) phenyl]pyrido[3,2-d]pyrimidine-8-carboxamide: The crude product (4-[(5-fluoropiperidin-3-yl)amino]-6-[4-(morpholin-4-ylmethyl)phenyl]pyrido[3,2-d]pyrimidine-8-carboxamide (135.00 mg, 0.290 mmol) was purified by Chiral-Prep-HPLC with the following conditions (Agela High-pressure Flash): Column, CHIRALPAK IG, 3*25 cm, 5 μm; mobile phase, MTBE (10 mM NH₃-MEOH) and EtOH (hold 50% EtOH in 30 min.); Detector, UV 254 nm; to afford 4-[[(3S,5R)-5-fluoropiperidin-3-yl]amino]-6-[4-(morpholin-4-ylmethyl)phenyl]pyrido[3,2-d]pyrimidine-8-carboxamide (24.1 mg, 17.25%, white solid), 4-[[(3S,5S)-5-fluoropiperidin-3-yl]amino]-6-[4-(morpholin-4-ylmethyl)phenyl]pyrido[3,2-d]pyrimidine-8-carboxamide (16.4 mg, 11.41%, white solid), 4-[[(3R,5R)-5-fluoropiperidin-3-yl]amino]-6-[4-(morpholin-4-ylmethyl)phenyl]pyrido[3,2-d]pyrimidine-8-carboxamide (11.4 mg, 7.72%, white solid) and 4-[[(3R,5S)-5-fluoropiperidin-3-yl]amino]-6-[4-(morpholin-4-ylmethyl)phenyl]pyrido[3,2-d]pyrimidine-8-carboxamide (23.5 mg, 17.29%, white solid).

Isomer 1: HPLC: 96.6% purity, RT=2.37 min. MS: m/z=466.2 [M+H]⁺. ¹H NMR (300 MHz, DMSO-d6, ppm) 10.02 (d, J=3.7 Hz, 1H), 8.85 (s, 1H), 8.59 (s, 1H), 8.53 (d, J=8.8 Hz, 1H), 8.34-8.29 (m, 2H), 8.23 (d, J=3.6 Hz, 1H), 7.52 (d, J=8.1 Hz, 2H), 4.83-4.58 (m, 1H), 4.41 (d, J=7.6 Hz, 1H), 3.60 (dd, J=9.4, 4.8 Hz, 6H), 3.14-2.95 (m, 2H), 2.77-2.64 (m, 2H), 2.41 (t, J=4.7 Hz, 4H), 2.30 (d, J=19.3 Hz, 1H), 2.16-2.03 (m, 1H).

Isomer 2: HPLC: 93.9% purity, RT=2.92 min. MS: m/z=466.2 [M+H]⁺. ¹H NMR (300 MHz, DMSO-d6, ppm) 10.01 (d, J=3.7 Hz, 1H), 8.82 (s, 1H), 8.57 (s, 1H), 8.33 (dd, J=12.2, 8.6 Hz, 3H), 8.20 (d, J=3.9 Hz, 1H), 7.49 (d, J=8.1 Hz, 2H), 4.94-4.55 (m, 2H), 3.57 (dd, J=9.5, 4.8 Hz, 7H), 3.10-2.91 (m, 2H), 2.81-2.71 (m, 1H), 2.66 (d, J=15.8 Hz, 1H), 2.38 (dd, J=5.6, 3.7 Hz, 4H), 2.25-2.09 (m, 2H).

Isomer 3: HPLC: 91.3% purity, RT=2.67 min. MS: m/z=466.3 [M+H]⁺. ¹H NMR (300 MHz, DMSO-d6, ppm) 10.04 (d, J=3.7 Hz, 1H), 8.85 (s, 1H), 8.59 (s, 1H), 8.36 (dd, J=14.4, 8.5 Hz, 3H), 8.23 (d, J=3.7 Hz, 1H), 7.51 (d, J=8.0 Hz, 2H), 4.87 (d, J=48.0 Hz, 1H), 4.61 (d, J=10.2 Hz, 1H), 3.60 (dd, J=9.7, 5.0 Hz, 6H), 3.09-2.96 (m, 2H), 2.83-2.69 (m, 2H), 2.40 (t, J=4.7 Hz, 4H), 2.25-2.09 (m, 2H).

Isomer 4: HPLC: 99.3% purity, RT=3.32 min. MS: m/z=466.2 [M+H]⁺. ¹H NMR (300 MHz, DMSO-d6, ppm) 10.02 (d, J=3.5 Hz, 1H), 8.85 (s, 1H), 8.65-8.49 (m, 2H), 8.35-8.21 (m, 3H), 7.52 (d, J=8.2 Hz, 2H), 4.83-4.60 (m, 1H), 4.47-4.36 (m, 1H), 3.60 (dd, J=9.5, 4.8 Hz, 6H), 3.15-2.94 (m, 2H), 2.77-2.64 (m, 2H), 2.41 (dd, J=5.7, 3.5 Hz, 5H), 2.09 (t, J=10.3 Hz, 1H).

### Example 27: Synthesis of Compound 27 6-(4-[[(2S)-2-methylmorpholin-4-yl]methyl]phenyl)-4-[(3S)-piperidin-3-ylamino]pyrido [3,2-d]pyrimidine-8 carboxamide dihydrochloride

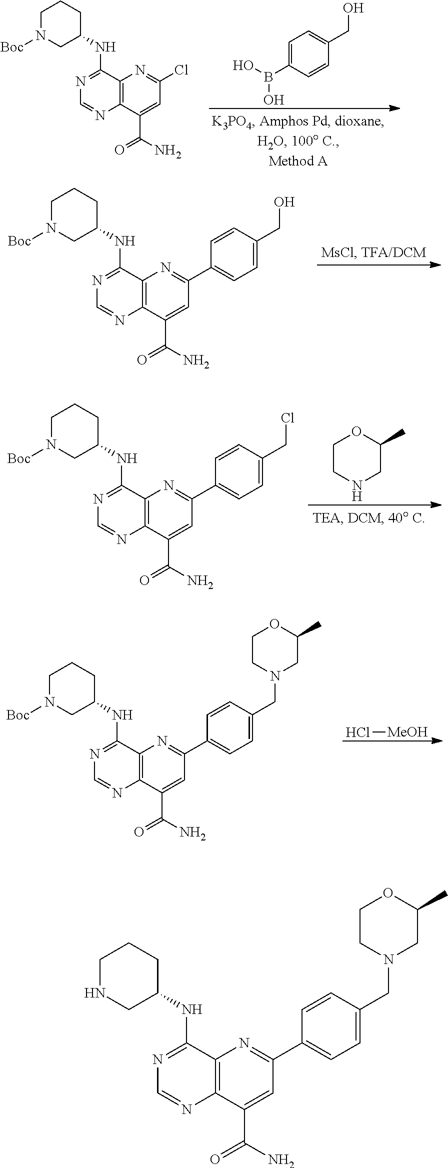

tert-butyl(3S)-3-([8-carbamoyl-6-[4-(hydroxymethyl)phenyl]pyrido[3,2-d]pyrimidin-4-yl]amino)piperidine-1-carboxylate To a solution of tert-butyl (3S)-3-([8-carbamoyl-6-chloropyrido[3,2-d]pyrimidin-4-yl]amino)piperidine-1-carboxylate (500.00 mg, 0.880 mmol) and 4-(hydroxymethyl)phenylboronic acid (224.00 mg, 1.400 mmol) in 1,4-Dioxane (8.00 mL) and H₂O (1.60 mL) were added K₃PO₄ (781.00 mg, 3.495 mmol, 3.97 equiv, 95.0%) and AMPHOS-PdCl₂ (87.00 mg, 0.117 mmol). After stirring for 2 h at 100° C. under a nitrogen atmosphere, the resulting mixture was concentrated under reduced pressure. The residue was purified by silica gel column chromatography, eluted with CH₂Cl₂/MeOH (10:1) to afford tert-butyl (3S)-3-([8-carbamoyl-6-[4-(hydroxymethyl)phenyl]pyrido[3,2-d]pyrimidin-4-yl]amino)piperidine-1-carboxylate (420 mg, 98.25%) as a yellow solid.

tert-butyl (3S)-3-([8-carbamoyl-6-[4-(chloromethyl)phenyl]pyrido[3,2-d]pyrimidin-4-yl]amino)piperidine-1-carboxylate: A solution of tert-butyl (3S)-3-([8-carbamoyl-6-[4-(hydroxymethyl)phenyl]pyrido[3,2-d]pyrimidin-4-yl]amino)piperidine-1-carboxylate (300.00 mg, 0.275 mmol), MsCl (109.19 mg, 0.906 mmol) and TEA (128.61 mg, 1.207 mmol) in DCM (4.00 mL, 47.098 mmol) was stirred for 2 h at 100° C. under nitrogen atmosphere. The resulting mixture was concentrated under vacuum. This resulted in tert-butyl (3S)-3-([8-carbamoyl-6-[4-(chloromethyl) phenyl]pyrido[3,2-d]pyrimidin-4-yl]amino)piperidine-1-carboxylate (250 mg, 56.08%) as a yellow solid.

tert-butyl(3S)-3-[[8-carbamoyl-6-(4-[[(2S)-2-methylmorpholin-4-yl]methyl]phenyl)pyrido[3,2-d]pyrimidin-4-yl]amino]piperidine-1-carboxylate: A solution of tert-butyl (3S)-3-([8-carbamoyl-6-[4-(chloromethyl)phenyl]pyrido[3,2-d]pyrimidin-4-yl]amino)piperidine-1-carboxylate (100.00 mg, 0.095 mmol) and (2S)-2-methylmorpholine (40.04 mg, 0.376 mmol), TEA (80.02 mg, 0.751 mmol) in DCM (2.00 mL, 23.548 mmol) was stirred for 3 h at 40° C. under nitrogen atmosphere. The resulting mixture was concentrated under vacuum. The residue was purified by silica gel column chromatography, and eluted with CH₂Cl₂/MeOH (1:1) to afford tert-butyl (3S)-3-[[8-carbamoyl-6-(4-[[(2S)-2-methylmorpholin-4-yl]methyl]phenyl)pyrido[3,2-d]pyrimidin-4-yl]amino]piperidine-1-carboxylate (50 mg, 93.73%) as a yellow oil.

6-(4-[[(2S)-2-methylmorpholin-4-yl]methyl]phenyl)-4-[(3S)-piperidin-3-ylamino]pyrido[3,2-d]pyrimidine-8-carboxamide dihydrochloride: A solution of tert-butyl(3S)-3-[[8-carbamoyl-6-(4-[[(2S)-2-methylmorpholin-4-yl]methyl]phenyl)pyrido[3,2-d]pyrimidin-4-yl]amino]piperidine-1-carboxylate (45.00 mg, 0.080 mmol) in HCl (gas) in 1,4-Dioxane (1.00 mL, 12%) was stirred for 2 h at 25° C. under nitrogen atmosphere. The resulting mixture was concentrated under vacuum. The crude product was purified by Prep-HPLC with the following conditions (2#SHIMADZU (HPLC-01)): Column, XBridge Shield RP18 OBD Column, 30*150 mm, 5 μm; mobile phase, water (0.05% HCl) and ACN (35% Phase B up to 55% in 8 min.); Detector, UV 254 nm. This resulted in 6-(4-[[(2S)-2-methylmorpholin-4-yl]methyl]phenyl)-4-[(3S)-piperidin-3-ylamino]pyrido[3,2-d]pyrimidine-8-carboxamide dihydrochloride (5.9 mg, 12.40%) as a yellow solid.

HPLC: 95.0% purity, RT=4.52 min. MS: m/z=462.0 [M+H]⁺. ¹H NMR (400 MHz, DMSO-d6) δ 8.95 (s, 1H), 8.69 (s, 1H), 8.55 (d, J=8.0 Hz, 2H), 7.84 (d, J=8.1 Hz, 2H), 4.74 (d, J=10.6 Hz, 1H), 4.54-4.28 (m, 2H), 4.05-3.76 (m, 3H), 3.35-3.16 (m, 5H), 3.06 (t, J=11.2 Hz, 1H), 2.82 (q, J=13.2, 12.0 Hz, 2H), 2.09-1.64 (m, 4H), 1.11 (d, J=6.3 Hz, 3H).

### Example 28: Synthesis of Compound 29 6-[4-[1-(morpholin-4-yl) cyclopropyl]phenyl]-4-[(3S)-piperidin-3-ylamino]pyrido [3,2-d]pyrimidine-8-carboxamide

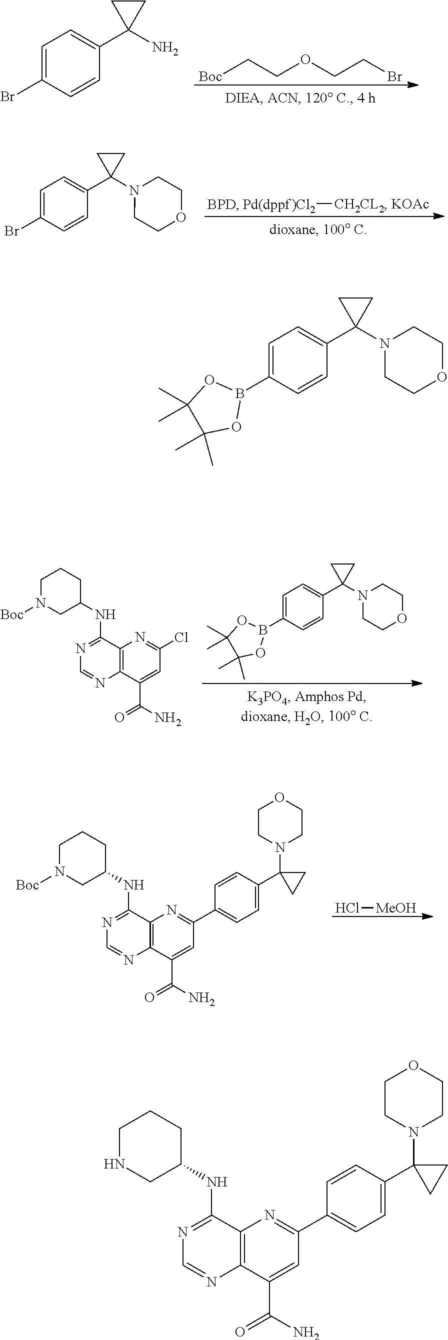

4-[1-(4-bromophenyl) cyclopropyl]morpholine: Into a 30-mL sealed tube, was placed 1-(4-bromophenyl) cyclopropan-1-amine (1 g, 4.479 mmol), MeCN (20.00 mL, 462.835 mmol), DIEA (3.05 g, 22.419 mmol), 1-bromo-2-(2-bromoethoxy) ethane (1.31 g, 5.366 mmol). The resulting solution was stirred for 4 h at 120° C. in an oil bath. The resulting mixture was concentrated. The residue was applied onto a silica gel column with ethyl acetate/petroleum ether (1:10-1:4). This resulted in 4-[1-(4-bromophenyl) cyclopropyl]morpholine (800 mg, 63.23%) as an off-white solid.

4-[1-[4-(4,4,5,5-tetramethyl-1,3,2-dioxaborolan-2-yl) phenyl]cyclopropyl]morpholine: Into a 30-mL sealed tube purged and maintained with an inert atmosphere of nitrogen was placed 4-[1-(4-bromophenyl) cyclopropyl]morpholine (800 mg, 2.832 mmol), 1,4-Dioxane (15.00 mL, 161.736 mmol), BPD (1135.60 mg, 4.248 mmol), KOAc (877.77 mg, 8.497 mmol), Pd(dppf)Cl₂·CH₂Cl₂ (243.46 mg, 0.283 mmol). The mixture was stirred overnight at 100° C. in an oil bath. The resulting mixture was concentrated. The residue was applied onto a silica gel column with ethyl acetate/petroleum ether (1:10-1:4). This resulted in 4-[1-[4-(4,4,5,5-tetramethyl-1,3,2-dioxaborolan-2-yl) phenyl]cyclopropyl]morpholine (700 mg, 71.01%) as a light brown solid.

tert-butyl (3S)-3-[(8-carbamoyl-6-[4-[1-(morpholin-4-yl)cyclopropyl]phenyl]pyrido[3,2-d]pyrimidin-4-yl)amino]piperidine-1-carboxylate: To a solution of tert-butyl (3S)-3-([8-carbamoyl-6-chloropyrido[3,2-d]pyrimidin-4-yl]amino)piperidine-1-carboxylate (80.00 mg, 0.138 mmol) and 4-[1-[4-(4,4,5,5-tetramethyl-1,3,2-dioxaborolan-2-yl)phenyl]cyclopropyl]morpholine (150.00 mg, 0.431 mmol) in 1,4-Dioxane (8.00 mL, 94.433 mmol) and H₂O (1.43 mL, 79.377 mmol) were added K₃PO₄ (120.00 mg, 0.537 mmol) and AMPHOS-PdCl₂ (16.00 mg, 0.021 mmol). The resulting mixture was stirred for 2 h at 100° C. under a nitrogen atmosphere. The resulting mixture was concentrated under vacuum. The residue was purified by silica gel column chromatography, eluted with DCM/MeOH (12:1) to afford tert-butyl (3S)-3-[(8-carbamoyl-6-[4-[1-(morpholin-4-yl) cyclopropyl]phenyl]pyrido[3,2-d]pyrimidin-4-yl)amino]piperidine-1-carboxylate (50 mg, 63.05%) as a brown solid.

6-[4-[1-(morpholin-4-yl) cyclopropyl]phenyl]-4-[(3S)-piperidin-3-ylamino]pyrido[3,2-d]pyrimidine-8-carboxamide: A solution of tert-butyl (3S)-3-[(8-carbamoyl-6-[4-[1-(morpholin-4-yl) cyclopropyl]phenyl]pyrido[3,2-d]pyrimidin-4-yl)amino]piperidine-1 carboxylate (40.00 mg, 0.044 mmol) in HCl (gas) in 1,4-Dioxane (5.00 mL, 16.456 mmol) was stirred for 1 h at room temperature. The resulting mixture was concentrated under vacuum. The crude product was purified by Prep-HPLC with the following conditions (2#SHIMADZU (HPLC-01)): Column, XBridge Shield RP18 OBD Column, 5 μm, 19*150 mm; mobile phase, water (10 mmoL/L NH₄HCO₃+0.1% [28% NH₃ in H₂O]) and ACN (25% Phase B up to 55% in 8 min.); Detector, UV 254 nm. 6-[4-[1-(morpholin-4-yl) cyclopropyl]phenyl]-4-[(3S)-piperidin-3-ylamino]pyrido[3,2-d]pyrimidine-8-carboxamide (10 mg, 44.95%) was obtained as a yellow solid.

HPLC: 93.5% purity, RT=4.03 min. MS: m/z=474.3 [M+H]⁺. ¹H NMR (300 MHz, Methanol-d4, ppm) 8.96 (d, J=0.8 Hz, 1H), 8.56 (d, J=0.7 Hz, 1H), 8.28 (d, J=8.2 Hz, 2H), 7.51 (d, J=8.3 Hz, 2H), 4.45-4.37 (m, 1H), 3.64 (t, J=4.6 Hz, 4H), 3.27 (d, J=3.9 Hz, 1H), 2.99 (d, J=12.7 Hz, 1H), 2.86-2.71 (m, 2H), 2.61 (t, J=4.7 Hz, 4H), 2.14 (s, 1H), 1.92-1.68 (m, 3H), 1.06 (q, J=4.1, 3.6 Hz, 2H), 1.01-0.86 (m, 2H).

### Example 29: Synthesis of Compound 30 4-[[(3R)-4,4-difluoropiperidin-3-yl]amino]-6-(4-[[(3R,5S)-3,5-dimethylmorpholin-4-yl]methyl]phenyl)pyrido[3,2-d]pyrimidine-8-carboxamide

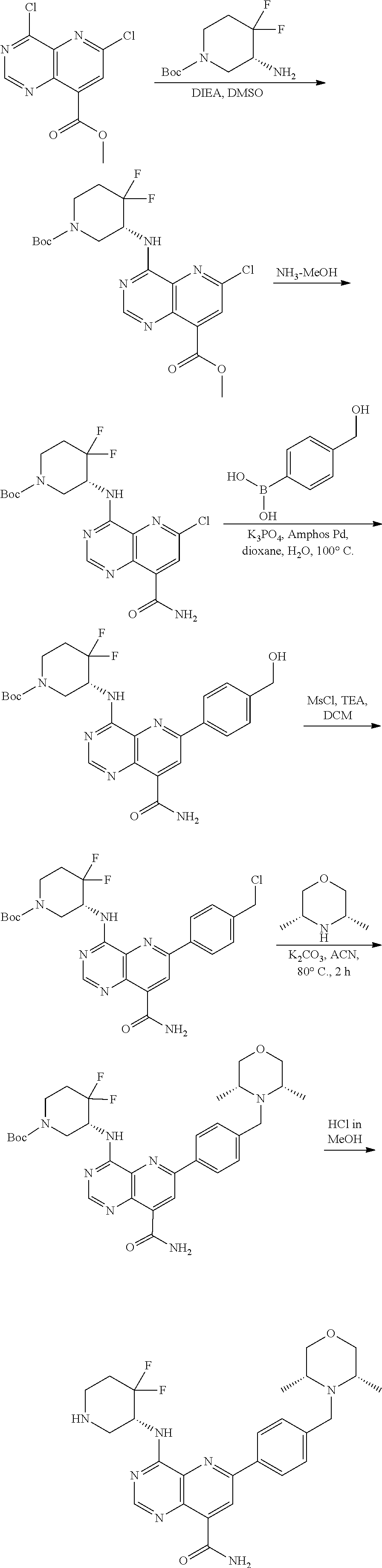

tert-butyl (3R)-3-[[6-chloro-8-(methoxycarbonyl)pyrido[3,2-d]pyrimidin-4-yl]amino]-4,4-difluoropiperidine-1-carboxylate: To a stirred solution of methyl 4,6-dichloropyrido[3,2-d]pyrimidine-8-carboxylate (1.5 g, 5.150 mmol) and tert-butyl (3R)-3-amino-4,4-difluoropiperidine-1-carboxylate (1.536 mg, 6.180 mmol) in DMSO (15.00 mL) was added DIEA (2.802 g, 20.600 mmol) at room temperature under nitrogen atmosphere. The resulting mixture was stirred for 3 h at 40° C. under nitrogen atmosphere. The reaction was quenched by the addition of water (60 mL) at room temperature. The resulting mixture was extracted with EtOAc (3×60 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EtOAc (7:3) to afford tert-butyl (3R)-3-[[6-chloro-8-(methoxycarbonyl)pyrido[3,2-d]pyrimidin-4-yl]amino]-4,4-difluoropiperidine-1-carboxylate (1.56 g, 65.70%) as a yellow solid.

tert-butyl (3R)-3-([8-carbamoyl-6-chloropyrido[3,2-d]pyrimidin-4-yl]amino)-4,4-difluoropiperidine-1-carboxylate: A solution of tert-butyl (3R)-3-[[6-chloro-8-(methoxycarbonyl)pyrido[3,2-d]pyrimidin-4-yl]amino]-4,4-difluoropiperidine-1-carboxylate (1.50 g, 1.727 mmol) in NH₃ (g) in MeOH (30.00 mL) was stirred for 1 h at 40° C. The resulting mixture was concentrated under vacuum to afford tert-butyl (3R)-3-([8-carbamoyl-6-chloropyrido[3,2-d]pyrimidin-4-yl]amino)-4,4-difluoropiperidine-1-carboxylate (1.0 g, crude product, 54.80%) as a brown solid.

tert-butyl (3R)-3-([8-carbamoyl-6-[4-(hydroxymethyl)phenyl]pyrido[3,2-d]pyrimidin-4-yl]amino)-4,4-difluoropiperidine-1-carboxylate: A solution of tert-butyl (3R)-3-([8-carbamoyl-6-chloropyrido[3,2-d]pyrimidin-4-yl]amino)-4,4-difluoropiperidine-1-carboxylate (840.00 mg, 1.584 mmol) and 4-(hydroxymethyl)phenylboronic acid (435.00 mg, 2.719 mmol), AMPHOS-PdCl₂ (134.00 mg, 0.180 mmol), K₃PO₄ (1.20 g, 5.369 mmol) in 1,4-Dioxane (10.00 mL) and H₂O (2.00 mL) was stirred for 2 h at 100° C. under nitrogen atmosphere. The resulting mixture was concentrated under vacuum. The residue was purified by silica gel column chromatography, eluted with PE/EtOAc (1:1) to afford tert-butyl (3R)-3-([8-carbamoyl-6-[4-(hydroxymethyl)phenyl]pyrido[3,2-d]pyrimidin-4-yl]amino)-4,4-difluoropiperidine-1-carboxylate (830 mg, 99.91%) as a brown solid.

tert-butyl (3R)-3-([8-carbamoyl-6-[4-(chloromethyl)phenyl]pyrido[3,2-d]pyrimidin-4-yl]amino)-4,4-difluoropiperidine-1-carboxylate: To a stirred solution of tert-butyl (3R)-3-([8-carbamoyl-6-[4-(hydroxymethyl)phenyl]pyrido[3,2-d]pyrimidin-4-yl]amino)-4,4-difluoropiperidine-1-carboxylate (240.00 mg, 0.448 mmol) and TEA (0.13 mL, 0.889 mmol) in DCM (6.00 mL) was added MsCl (81.08 mg, 0.672 mmol) dropwise at 0° C. under argon atmosphere. The resulting mixture was stirred for overnight at room temperature under argon atmosphere. The resulting mixture was concentrated under vacuum. This resulted in tert-butyl (3R)-3-([8-carbamoyl-6-[4-(chloromethyl)phenyl]pyrido[3,2-d]pyrimidin-4-yl]amino)-4,4-difluoropiperidine-1-carboxylate (210 mg, 58.72%) as a light-yellow solid.

tert-butyl (3R)-3-[[8-carbamoyl-6-(4-[[(3R,5S)-3,5-dimethylmorpholin-4-yl]methyl]phenyl)pyrido[3,2-d]pyrimidin-4-yl]amino]-4,4-difluoropiperidine-1-carboxylate: A solution of tert-butyl (3R)-3-([8-carbamoyl-6-[4-(chloromethyl)phenyl]pyrido[3,2-d]pyrimidin-4-yl]amino)-4,4-difluoropiperidine-1-carboxylate (150.00 mg, 0.134 mmol) and (3R,5S)-3,5-dimethylmorpholine (46.71 mg, 0.385 mmol), K₂CO₃ (77.92 mg, 0.536 mmol) in ACN (2.00 mL) was stirred for 32 h at 70° C. under nitrogen atmosphere. The residue was purified by silica gel column chromatography, eluted with PE/EtOAc (1:10) to afford tert-butyl (3R)-3-[[8-carbamoyl-6-(4-[[(3R,5S)-3,5-dimethylmorpholin-4-yl]methyl]phenyl)pyrido[3,2-d]pyrimidin-4-yl]amino]-4,4-difluoropiperidine-1-carboxylate (60 mg, 70.29%) as a yellow oil.

4-[[(3R)-4,4-difluoropiperidin-3-yl]amino]-6-(4-[[(3R,5S)-3,5-dimethylmorpholin-4-yl]methyl]phenyl)pyrido[3,2-d]pyrimidine-8-carboxamide: A solution of tert-butyl (3R)-3-[[8-carbamoyl-6-(4-[[(3R,5S)-3,5-dimethylmorpholin-4-yl]methyl]phenyl)pyrido[3,2-d]pyrimidin-4-yl]amino]-4,4-difluoropiperidine-1-carboxylate (55.00 mg, 0.086 mmol) and HCl (gas) in 1,4-Dioxane (2.00 mL) was stirred for 2 h at 25° C. under nitrogen atmosphere. The precipitated solids were collected by filtration and washed with 1,4-Dioxane (3×10 mL). The crude solid was dissolved in water and basified to pH=9 with saturated NaHCO₃ (aq.). The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure. This resulted in 4-[[(3R)-4,4-difluoropiperidin-3-yl]amino]-6-(4-[[(3R,5S)-3,5-dimethylmorpholin-4-yl]methyl]phenyl)pyrido[3,2-d]pyrimidine-8-carboxamide (19.6 mg, 43.85%) as a white solid.

HPLC: 99.0% purity, RT=4.429 min. MS: m/z=512.25 [M+H]⁺. ¹H NMR (300 MHz, DMSO-d6) δ 9.89 (s, 1H), 8.85 (s, 1H), 8.65 (s, 1H), 8.32-8.15 (m, 4H), 7.59 (d, J=8.0 Hz, 2H), 4.95-4.80 (m, 1H), 3.84 (s, 2H), 3.71-3.67 (s, 1H), 3.63-3.61 (m, 1H), 3.20-3.07 (m, 1H), 2.98 (d, J=11.4 Hz, 3H), 2.83-2.63 (m, 2H), 2.71 (s, 5H), 0.88 (d, J=6.2 Hz, 6H).

### Example 30: Synthesis of Compound 31 6-[4-(morpholin-4-ylmethyl) phenyl]-4-[(3S)-piperidin-3-ylamino]pyrido[3,2-d]pyrimidine-8-carboxamide

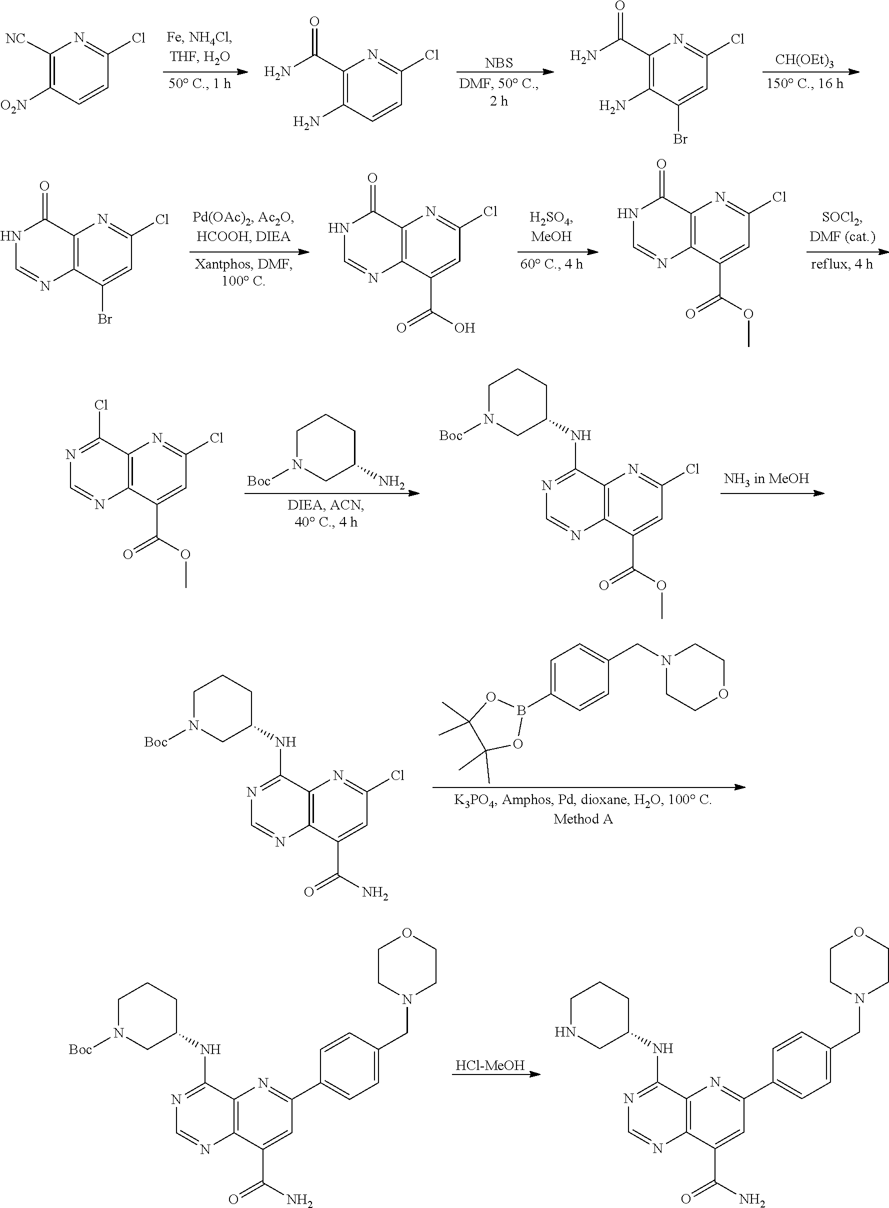

3-amino-6-chloropyridine-2-carboxamide: A mixture of 6-chloro-3-nitropyridine-2-carbonitrile (182.00 g, 793.244 mmol) in THF [tetrahydrofuran](1800.11 mL) was added NH₄Cl (210 g, 37391.034 mmol) in water (2000 mL) and Fe (336.20 g, 5719.223 mmol,) at room temperature. The mixture was stirred for 1 h at 50° C. The resulting mixture was filtered, the filter cake was washed with EtOAc (2×2000 mL). The combined organic layers were washed with NaHCO₃ (aq.) and brine (2×3000 mL), dried over anhydrous Na₂SO₄. After filtration, the filtrate was concentrated under reduced pressure. This resulted in 3-amino-6-chloropyridine-2-carboxamide (137 g, 79.82%) as a Light reddish brown solid.

3-amino-4-bromo-6-chloropyridine-2-carboxamide: To a stirred mixture of 3-amino-6-chloropyridine-2-carboxamide (50.00 g, 276.839 mmol) in DMF (500.00 mL, 6460.876 mmol) was added NBS (62.24 g, 332.207 mmol) in portions at room temperature. The resulting mixture was stirred for 3 h at 50° C. under a nitrogen atmosphere. The resulting mixture was filtered, the filter cake was washed with PE (3×500 mL). The organic layer was diluted with water at room temperature. The resulting mixture was extracted with EtOAc (3×1500 mL). The combined organic layers were washed with brine (4×1000 mL), dried over anhydrous Na₂SO₄. After filtration, the filtrate was concentrated under reduced pressure. The residue was purified by silica gel column chromatography, eluted with PE/EtOAc (3:2) to afford 3-amino-4-bromo-6-chloropyridine-2-carboxamide as a brown solid (47 g, 61.20%).

8-bromo-6-chloropyrido[3,2-d]pyrimidin-4 (3H)-one: A solution of 3-amino-4-bromo-6-chloropyridine-2-carboxamide (65.00 g; 232.25 mmol) in triethyl orthoformate (600.00 ml) was stirred for 16 h at 150° C. under nitrogen atmosphere. The precipitated solids were collected by filtration and washed with PE (3×1000 mL). The resulting solid was dried in an oven under reduced pressure. This resulted in 8-bromo-6-chloropyrido[3,2-d]pyrimidin-4 (3H)-one (45.00 g, crude product) as a yellow solid.

6-chloro-4-oxo-3H,4H-pyrido[3,2-d]pyrimidine-8-carboxylic acid: To a flask was added Ac₂O (100.00 ml) and HCOOH (20.71 ml; 521.51 mmol). The mixture was heated to 50° C. for 30 min. The resulting mixture was added to a flask with 8-bromo-6-chloro-3H,4H-pyrido[3,2-d]pyrimidin-4-one (25.00 g; 86.92 mmol), Pd(AcO)₂ (1.08 g; 4.35 mmol), XantPhos (5.29 g; 8.69 mmol) and DIEA (17.73 g; 130.38 mmol) in DMF (200.00 ml) slowly at 75° C. The resulting mixture was heated to 100° C. and stirred overnight. The mixture was cooled to room temperature. The pH of the mixture was adjusted to 10-11 by NaOH (aq. 4N) at 0° C. The solid was removed by filtration. The filter was extracted by EtOAc (500 mL×3). Then the aqueous layer was adjusted pH to 2 and extracted with EA. The organic layers were dried over Na₂SO₄ and concentrated under vacuum. This resulted in 6-chloro-4-oxo-3H,4H-pyrido[3,2-d]pyrimidine-8-carboxylic acid (12.40 g, crude product) as a brown dark oil.

methyl 6-chloro-4-oxo-3H-pyrido[3,2-d]pyrimidine-8-carboxylate: To a stirred solution of 6-chloro-4-oxo-3H-pyrido[3,2-d]pyrimidine-8-carboxylic acid (7.30 g, 25.370 mmol) in MeOH (70.00 mL, 2184.685 mmol) was added H₂SO₄ (0.50 mL, 9.193 mmol) dropwise at room temperature. The mixture was stirred for 4-8 h at 60° C. When the reaction was done it was quenched by the addition of ice-cold water (200 mL). The precipitated solids were collected by filtration and dried to afford methyl 6-chloro-4-oxo-3H-pyrido[3,2-d]pyrimidine-8-carboxylate (6.5 g, 55.60%) as a yellow solid.

methyl 4,6-dichloropyrido[3,2-d]pyrimidine-8-carboxylate: To a stirred solution of methyl 6-chloro-4-oxo-3H-pyrido[3,2-d]pyrimidine-8-carboxylate (7.30 g, 15.842 mmol) in SOCl₂ (50.00 mL, 420.289 mmol) was added DMF (3 drops) at room temperature. The resulting mixture was stirred for 3 h at 80° C. The resulting mixture was concentrated under vacuum to afford methyl 4,6-dichloropyrido[3,2-d]pyrimidine-8-carboxylate (6.5 g, 93.17%) as a yellow solid.

tert-butyl (3S)-3-[[6-chloro-8-(methoxycarbonyl) pyrido[3,2-d]pyrimidin-4-yl]amino]piperidine-1-carboxylate: A solution of methyl 4,6-dichloropyrido[3,2-d]pyrimidine-8-carboxylate (5.30 g, 14.890 mmol), tert-butyl (3S)-3-aminopiperidine-1-carboxylate (4.90 g, 23.242 mmol) and DIEA (13.00 g, 95.556 mmol) in CH₃CN (50.00 mL) was stirred for 1 h at 40° C. The resulting mixture was concentrated under vacuum. The residue was purified by silica gel column chromatography, eluted with PE/EtOAc (1:1) to afford tert-butyl (3S)-3-[[6-chloro-8-(methoxycarbonyl) pyrido[3,2-d]pyrimidin-4-yl]amino]piperidine-1-carboxylate (7.8 g, 96.73%) as a yellow solid.

tert-butyl (3S)-3-([8-carbamoyl-6-chloropyrido[3,2-d]pyrimidin-4-yl]amino) piperidine-1-carboxylate: A solution of tert-butyl (3S)-3-[[6-chloro-8-(methoxycarbonyl)pyrido[3,2-d]pyrimidin-4-yl]amino]piperidine-1-carboxylate (6.10 g, 11.264 mmol) in NH₃ (g) in MeOH (20.00 mL, 152.622 mmol, 7 mol/L) was stirred for 2 h at room temperature. The resulting mixture was concentrated under vacuum to afford tert-butyl (3S)-3-([8-carbamoyl-6-chloropyrido[3,2-d]pyrimidin-4-yl]amino)piperidine-1-carboxylate (6.5 g, 88.08%) as a yellow solid.

tert-butyl (3S)-3-([8-carbamoyl-6-[4-(morpholin-4-ylmethyl)phenyl]pyrido[3,2-d]pyrimidin-4-yl]amino)piperidine-1-carboxylate: To a solution of tert-butyl (3S)-3-([8-carbamoyl-6-chloropyrido[3,2-d]pyrimidin-4-yl]amino)piperidine-1-carboxylate (7.50 g, 17.198 mmol) and 4-[[4-(4,4,5,5-tetramethyl-1,3,2-dioxaborolan-2-yl)phenyl]methyl]morpholine (5.98 g, 18.736 mmol) in 1,4-Dioxane (50.00 mL) and H₂O (10.00 mL, 555.084 mmol, 32.28 equiv, 100%) were added K₃PO₄ (11.26 g, 50.391 mmol) and AMPHOS-PdCl₂ (1.54 g, 0.003 mmol). The resulting mixture was stirred for 2 h at 100° C. under nitrogen atmosphere. The resulting mixture was cooled down to room temperature, diluted with water, and extracted with EtOAc (3×300 mL). The combined organic layers were washed with brine (3×400 mL), and dried over anhydrous Na₂SO₄. After filtration, the filtrate was concentrated under reduced pressure. The residue was purified by reverse flash chromatography with the following conditions: column, C18 silica gel; mobile phase, ACN in water, 60% to 70% gradient in 10 min; detector, UV 254 nm. This resulted in tert-butyl (3S)-3-([8-carbamoyl-6-[4-(morpholin-4-ylmethyl) phenyl]pyrido[3,2-d]pyrimidin-4-yl]amino)piperidine-1-carboxylate (6.5 g, 69.01%) as a yellow solid.

6-[4-(morpholin-4-ylmethyl) phenyl]-4-[(3S)-piperidin-3-ylamino]pyrido[3,2-d]pyrimidine-8-carboxamide: A mixture of tert-butyl (3S)-3-([8-carbamoyl-6-[4-(morpholin-4-ylmethyl)phenyl]pyrido[3,2-d]pyrimidin-4-yl]amino)piperidine-1-carboxylate (7.00 g, 12.782 mmol) in HCl (gas) in 1,4-Dioxane (50.00 mL, 12%) was stirred for 2 h at room temperature. The precipitated solids were collected by filtration and washed with 1,4-Dioxane (3×100 mL). The crude solid was dissolved in water and basified to pH=9 with saturated NaHCO₃ (aq.). The resulting mixture was extracted with CH₂Cl₂ (3×100 mL). The combined organic layers were washed with brine (1×100 mL), dried over anhydrous Na₂SO₄. After filtration, the filtrate was concentrated under reduced pressure. This resulted in 6-[4-(morpholin-4-ylmethyl) phenyl]-4-[(3S)-piperidin-3-ylamino]pyrido [3,2-d]pyrimidine-8-carboxamide (5.5658 g, 96.91%) as a yellow solid.

HPLC: 99.6% purity, RT=3.580 min. MS: m/z=448.1 [M+H]⁺. ¹H NMR (400 MHz, DMSO-d6, ppm) δ 10.06 (d, J=3.7 Hz, 1H), 8.83 (s, 1H), 8.56 (s, 1H), 8.36 (d, J=8.6 Hz, 1H), 8.27 (dd, J=16.1, 5.8 Hz, 3H), 7.50 (d, J=7.9 Hz, 2H), 4.30 (m, J=8.8, 4.8 Hz, 1H), 3.60 (t, J=4.6 Hz, 4H), 3.56 (s, 2H), 3.05 (dd, J=12.0, 3.8 Hz, 1H), 2.82 (m, J=12.6, 4.2 Hz, 1H), 2.74 (dd, J=11.7, 8.6 Hz, 1H), 2.67 (s, 1H), 2.58 (t, J=10.3 Hz, 1H), 2.39 (t, J=4.6 Hz, 4H), 1.94-1.88 (m, 1H), 1.82 (t, J=8.9, 4.4 Hz, 1H), 1.69 (d, J=13.6 Hz, 1H), 1.56-1.43 (m; 1H).

### Example 31: Synthesis of Compound 32 6-[4-(morpholin-4-ylmethyl) phenyl]-4-[(3S)-piperidin-3-ylamino]pyrido [3,2-d]pyrimidine-8-carboxamide

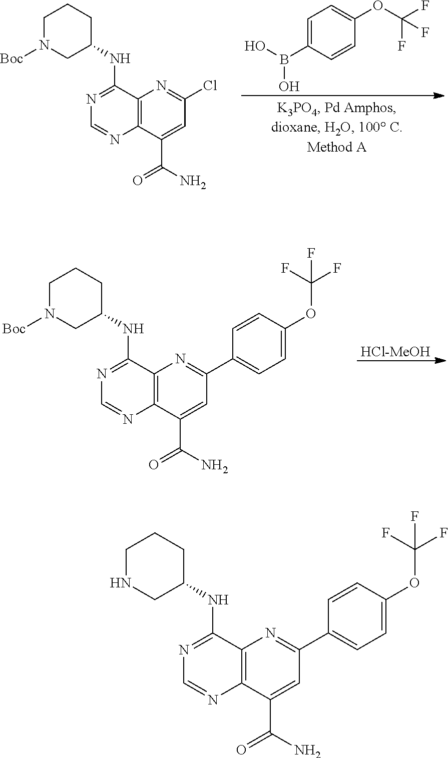 [SMILES: CC(C)(C)OC(=O)N1CCC[C@H](Nc2ncnc3c(C(N)=O)cc(Cl)nc23)C1 ; OB(O)c1ccc(OC(F)(F)F)cc1 ; CC(C)(C)OC(=O)N1CCC[C@H](Nc2ncnc3c(C(N)=O)cc(-c4ccc(OC(F)(F)F)cc4)nc23)C1 ; NC(=O)c1cc(-c2ccc(OC(F)(F)F)cc2)nc2c(N[C@H]3CCCNC3)ncnc12 ; "HCl-MeOH"]

tert-butyl (3S)-3-([8-carbamoyl-6-[4-(trifluoromethoxy) phenyl]pyrido [3,2-d]pyrimidin-4-yl]amino)piperidine-1-carboxylate: To a solution of tert-butyl (3S)-3-([8-carbamoyl-6-chloropyrido[3,2-d]pyrimidin-4-yl]amino)piperidine-1-carboxylate (5.00 g, 11.969 mmol) and 4-(trifluoromethoxy) phenylboronic acid (3113.51 mg, 14.363 mmol) in 1,4-Dioxane (50.00 mL) and H₂O (10.00 mL) were added K₃PO₄ (8023.28 mg, 35.908 mmol) and AMPHOS-PdCl₂ (892.13 mg, 1.197 mmol). The resulting mixture is stirred for 2 h at 100° C. under a nitrogen atmosphere, the resulting mixture was concentrated under reduced pressure. The residue was purified by silica gel column chromatography, eluted with PE/EtOAc (2:3) to afford tert-butyl (3S)-3-([8-carbamoyl-6-[4-(trifluoromethoxy) phenyl]pyrido[3,2-d]pyrimidin-4-yl]amino)piperidine-1-carboxylate (3.5 g, 53.81%) as a yellow solid.

4-[(3S)-piperidin-3-ylamino]-6-[4-(trifluoromethoxy) phenyl]pyrido[3,2-d]pyrimidine-8-carboxamide: A solution of tert-butyl (3S)-3-([8-carbamoyl-6-[4-(trifluoromethoxy) phenyl]pyrido[3,2-d]pyrimidin-4-yl]amino)piperidine-1-carboxylate (3.50 g, 6.099 mmol) in HCl (g) in MeOH (35.00 mL, 12%) was stirred for 2 h at room temperature. The resulting mixture was concentrated under vacuum. The crude product was re-crystallized from EtOAc (30 mL) to afford 4-[(3S)-piperidin-3-ylamino]-6-[4-(trifluoromethoxy)phenyl]pyrido[3,2-d]pyrimidine-8-carboxamide (1.9177 g, 68.03%) as a light-yellow solid.

HPLC: 98.8% purity, RT=5.54 min. MS: m/z=433.2 [M+H]⁺. ¹H NMR (300 MHz, DMSO-d6, ppm) δ 10.00 (d, J=3.6 Hz, 1H), 8.82 (s, 1H), 8.55 (s, 1H), 8.50-8.41 (m, 3H), 8.37-8.22 (m, 1H), 7.56-7.47 (m, 2H), 4.26 (m, J=8.9, 4.8 Hz, 1H), 3.03 (dd, J=12.0, 4.1 Hz, 1H), 2.85-2.69 (m, 2H), 2.60-2.50 (m, 1H), 1.91-1.74 (m, 3H), 1.74-1.61 (m, 1H).

### Example 32: 4-(((3S,5S)-5-fluoropiperidin-3-yl)amino)-6-(6-(3-hydroxypyrrolidin-1-yl)pyridin-3-yl)pyrido[3,2-d]pyrimidine-8-carboxamide

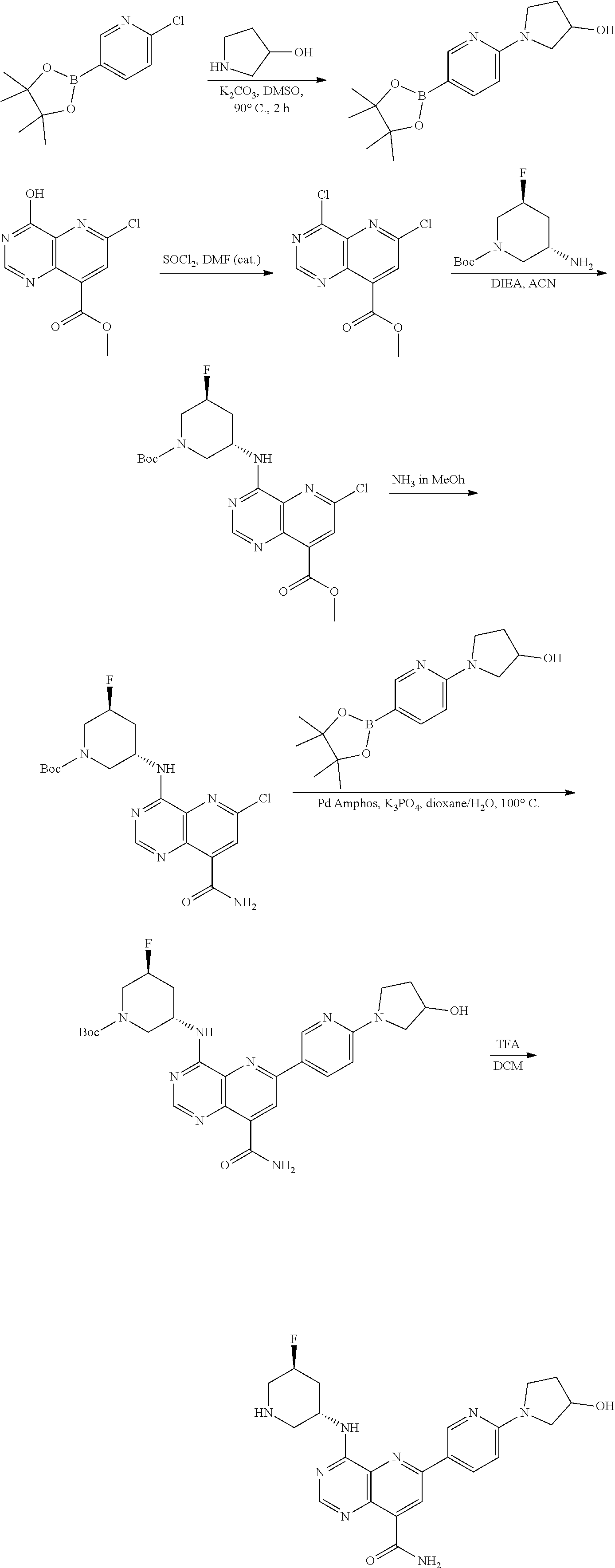

1-[5-(4,4,5,5-tetramethyl-1,3,2-dioxaborolan-2-yl)pyridin-2-yl]pyrrolidin-3-ol: To a stirred mixture of 2-chloro-5-(4,4,5,5-tetramethyl-1,3,2-dioxaborolan-2-yl)pyridine (1.20 g; 4.76 mmol) and pyrrolidin-3-ol (0.64 g; 7.14 mmol) in DMSO (20.00 ml) was added K₂CO₃ (1.38 g; 9.52 mmol) at room temperature. After stirring for 3 h at 90 degrees C. The resulting mixture was quenched with water and extracted with EtOAc. The combined organic layers were washed with brine, dried over anhydrous Na₂SO₄. After filtration, the filtrate was concentrated under reduced pressure. The resulting mixture was concentrated under reduced pressure and purified by silica gel column chromatography, eluted with CH₂Cl₂/MeOH (3:1) to afford 1-[5-(4,4,5,5-tetramethyl-1,3,2-dioxaborolan-2-yl)pyridin-2-yl]pyrrolidin-3-ol as brown oil (700 mg; 44.68%).

methyl 4,6-dichloropyrido[3,2-d]pyrimidine-8-carboxylate: To a solution of methyl 6-chloro-4-oxo-3H,4H-pyrido[3,2-d]pyrimidine-8-carboxylate (10 g; 12.80 mmol;) in Thionyl chloride (80 ml) was added DMF (0.5 ml) dropwise. The resulting mixture was stirred for 4 h at 80 degree (at this time the mixture became a clearly solution). The resulting mixture was concentrated under reduced pressure and co-evaporated with DCM 4 times. This resulted in methyl 4,6-dichloropyrido[3,2-d]pyrimidine-8-carboxylate as a light brown solid (8 g; 79.6%).

tert-butyl (3S,5S)-3-{[6-chloro-8-(methoxycarbonyl)pyrido[3,2-d]pyrimidin-4-yl]amino}-5-fluoropiperidine-1-carboxylate: To a stirred solution of methyl 4,6-dichloropyrido[3,2-d]pyrimidine-8-carboxylate (6.5 g; 22.67 mmol) and tert-butyl (3S,5S)-3-amino-5-fluoropiperidine-1-carboxylate (5.21 g; 22.67 mmol) in MeCN (60.00 ml) was added iPr2NEt (12.17 ml; 68.01 mmol) in portion at room temperature under N₂ atmosphere. The resulting mixture was stirred for 2 h at 40 degree C. under N₂ atmosphere. The resulting mixture was diluted with water (100 mL), extracted with EtOAc (3×200 mL). The combined organic layers were washed with brine (150 mL), dried over anhydrous Na₂SO₄. The resulting mixture was concentrated under reduced pressure to afford tert-butyl (3S,5S)-3-{[6-chloro-8-(methoxycarbonyl)pyrido[3,2-d]pyrimidin-4-yl]amino}-5-fluoropiperidine-1-carboxylate as brown solid (12.1 g; Crude Product).

tert-butyl (3S,5S)-3-({8-carbamoyl-6-chloropyrido[3,2-d]pyrimidin-4-yl}amino)-5-fluoropiperidine-1-carboxylate: To a stirred solution of tert-butyl (3S,5S)-3-{[6-chloro-8-(methoxycarbonyl)pyrido[3,2-d]pyrimidin-4-yl]amino}-5-fluoropiperidine-1-carboxylate (10 g; 18.26 mmol) in NH₃ in MeOH (50 ml; 350.00 mmol) was stirred for 4 hours at 40 degree C. The resulting mixture was concentrated under reduced pressure to afford tert-butyl (3S,5S)-3-({8-carbamoyl-6-chloropyrido[3,2-d]pyrimidin-4-yl}amino)-5-fluoropiperidine-1-carboxylate as brown solid (8.5 g; Crude Product).

tert-butyl (3S,5S)-3-({8-carbamoyl-6-[6-(3-hydroxypyrrolidin-1-yl)pyridin-3-yl]pyrido[3,2-d]pyrimidin-4-yl}amino)-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (150 mg; 0.28 mmol) and 1-[5-(4,4,5,5-tetramethyl-1,3,2-dioxaborolan-2-yl)pyridin-2-yl]pyrrolidin-3-ol (136.4 mg; 0.41 mmol) in Dioxane-1,4 (5 ml) and Water (1 ml) were added Bis(di-tert-butyl(4-dimethylaminophenyl)phosphine)dichloropalladium(II) (20.58 mg; 0.03 mmol) and K₃PO₄ (185.06 mg; 0.83 mmol) at room temperature. After stirring for 2 h at 100 degrees C. under argon atmosphere. The resulting mixture was concentrated under reduced pressure and purified by silica gel column chromatography, eluted with CH₂Cl₂/MeOH (9:1) to afford tert-butyl (3S,5S)-3-({8-carbamoyl-6-[6-(3-hydroxypyrrolidin-1-yl)pyridin-3-yl]pyrido[3,2-d]pyrimidin-4-yl}amino)-5-fluoropiperidine-1-carboxylate as yellow solid (130 mg; 64.11%).

4-(((3S,5S)-5-fluoropiperidin-3-yl)amino)-6-(6-(3-hydroxypyrrolidin-1-yl)pyridin-3-yl)pyrido[3,2-d]pyrimidine-8-carboxamide: To a solution of tert-butyl (3S,5S)-3-({8-carbamoyl-6-[6-(3-hydroxypyrrolidin-1-yl)pyridin-3-yl]pyrido[3,2-d]pyrimidin-4-yl}amino)-5-fluoropiperidine-1-carboxylate (120.00 mg; 0.16 mmol) in DCM (5.00 ml) were added TFA (1.00 ml). After stirring for 2 h at 25 degrees C. The resulting mixture was concentrated under reduced pressure. The residue was purified by Prep-HPLC (Column: Xselect CSH C18 OBD Column 30*150 mm 5 μm; Mobile Phase A: Water (10 mmol/L NH₄HCO₃+0.05% NH₄OH), Mobile Phase B: ACN; Flow rate: 60 mL/min; Gradient: 7% B to 37% B in 8 min, 37% B; Wave Length: 254/220 nm; RT1 (min): 5.6; Number Of Runs: 2) to afford 4-{[(3S,5S)-5-fluoropiperidin-3-yl]amino}-6-[6-(3-hydroxypyrrolidin-1-yl)pyridin-3-yl]pyrido[3,2-d]pyrimidine-8-carboxamide as yellow orange solid (28 mg; 35.2%)

HPLC 92% purity, 9.02 min. MS:m/z=453.20 [M+H]⁺.

1H NMR (400 MHz, DMSO-d6): 10.03 (d, J=3.7 Hz, 1H), 9.24 (d, J=2.5 Hz, 1H), 8.73 (s, 1H), 8.53 (s, 1H), 8.46 (dd, J=9.0, 2.5 Hz, 1H), 8.36 (d, J=9.0 Hz, 1H), 8.20 (d, J=3.9 Hz, 1H), 6.57 (d, J=9.0 Hz, 1H), 5.05 (s, 1H), 4.98 (s, 1H), 4.44 (s, 1H), 3.62-3.54 (m, 3H), 3.08 (dt, J=15.8, 7.9 Hz, 2H), 2.86-2.69 (m, 2H), 2.27-2.19 (m, 2H), 2.18-2.00 (m, 2H), 1.93 (s, 1H).

### Example 33: 6-(3,5-difluoro-4-((1-hydroxycyclobutyl)methoxy)phenyl)-4-(((3S,5S)-5-fluoropiperidin-3-yl)amino)pyrido[3,2-d]pyrimidine-8-carboxamide

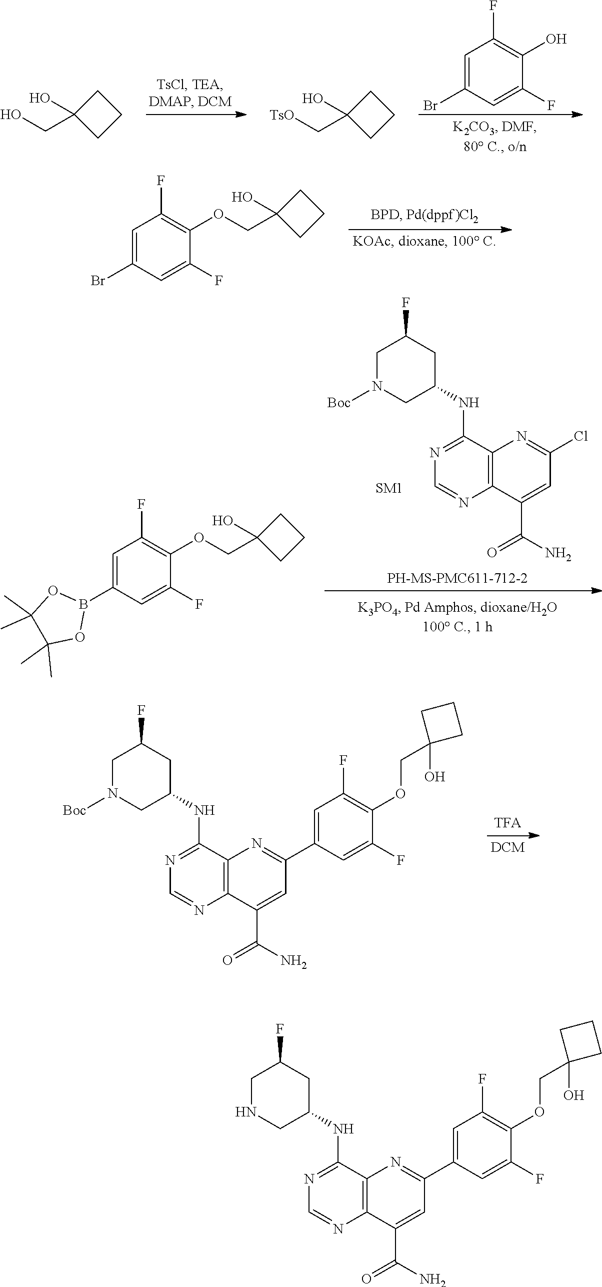

(1-hydroxycyclobutyl)methyl 4-methylbenzene-1-sulfonate: To a stirred mixture of 1-(hydroxymethyl)cyclobutan-1-ol (1 g; 9.30 mmol) in DCM (5 ml) was added TEA (4.09 ml; 27.98 mmol) and DMAP (0.12 g; 0.93 mmol) in portion at 0 degrees C., then to the mixture was added 4-methylbenzene-1-sulfonyl chloride (2.06 g; 10.26 mmol) in DCM (5.00 ml) dropwise at 0 degrees C. The resulting mixture was stirred for overnight at room temperature under nitrogen atmosphere. The reaction was quenched with water at 0 degrees C. The mixture was extracted with DCM (3×30 mL). The combined organic layers were washed with brine, dried over anhydrous Na₂SO₄. After filtration, the filtrate was concentrated under reduced pressure. The residue was purified by silica gel column chromatography, eluted with 15% EA in PE to afford (1-hydroxycyclobutyl)methyl 4-methylbenzene-1-sulfonate as white solid (1.8 g; 69.1%).

1-[(4-bromo-2,6-difluorophenoxy)methyl]cyclobutan-1-ol: To a stirred solution of (1-hydroxycyclobutyl)methyl 4-methylbenzene-1-sulfonate (900 mg; 3.21 mmol) and 4-bromo-2,6-difluorophenol (560 mg; 2.55 mmol) in DMF (5 ml) were added potassium carbonate (1.07 g; 7.35 mmol) in portion at room temperature under nitrogen atmosphere. The resulting mixture was stirred for overnight at 80 degrees C. under nitrogen atmosphere. The reaction was added water. The resulting mixture was extracted with EA (3×30 mL). The combined organic layers were washed with brine, dried over anhydrous Na₂SO₄. After filtration, the filtrate was concentrated under reduced pressure. The residue was purified by reverse flash chromatography with the following conditions: column, C18 silica gel; mobile phase, ACN in water, 55% to 65% gradient in 10 min; detector, UV 220 nm to afford 1-[(4-bromo-2,6-difluorophenoxy)methyl]cyclobutan-1-ol as yellow oil (550 mg; 55.6%).

1-{[2,6-difluoro-4-(4,4,5,5-tetramethyl-1,3,2-dioxaborolan-2-yl)phenoxy]methyl}cyclobutan-1-ol: To a stirred mixture of 1-[(4-bromo-2,6-difluorophenoxy)methyl]cyclobutan-1-ol (200 mg; 0.65 mmol) and BPD (245 mg; 0.92 mmol) in Dioxane-1,4 (5 ml) was added KOAc (165 mg; 1.60 mmol) and Pd(dppf)Cl₂ (50 mg; 0.06 mmol) in portion at room temperature. The resulting mixture was stirred for 1 h at 100 degrees C. under nitrogen atmosphere. The resulting mixture was concentrated to afford 1-{[2,6-difluoro-4-(4,4,5,5-tetramethyl-1,3,2-dioxaborolan-2-yl)phenoxy]methyl}cyclobutan-1-ol as brown solid (200 mg; Crude Product).

tert-butyl (3S,5S)-3-[(8-carbamoyl-6-{3,5-difluoro-4-[(1-hydroxycyclobutyl)methoxy]phenyl}pyrido[3,2-d]pyrimidin-4-yl)amino]-5-fluoropiperidine-1-carboxylate: To a stirred solution of tert-butyl (3S,5S)-3-({8-carbamoyl-6-chloropyrido[3,2-d]pyrimidin-4-yl}amino)-5-fluoropiperidine-1-carboxylate (140 mg; 0.26 mmol) and 1-{[2,6-difluoro-4-(4,4,5,5-tetramethyl-1,3,2-dioxaborolan-2-yl)phenoxy]methyl}cyclobutan-1-ol (176 mg; 0.25 mmol) in Dioxane-1,4 (4.5 ml) and Water (1.5 ml) were added Pd(AMPHOS)Cl₂ (195 mg; 0.26 mmol) and potassium phosphate tribasic (22 mg; 0.10 mmol) in portion at room temperature under N₂ atmosphere. The resulting mixture was stirred for 2 h at 100 degree C. under N₂ atmosphere. The resulting mixture was concentrated under reduced pressure. The residue was purified by silica gel column chromatography, eluted with 40% EA in PE to afford tert-butyl (3S,5S)-3-[(8-carbamoyl-6-{3,5-difluoro-4-[(1-hydroxycyclobutyl)methoxy]phenyl}pyrido[3,2-d]pyrimidin-4-yl)amino]-5-fluoropiperidine-1-carboxylate as yellow orange solid (200 mg; 87.3%).

6-{3,5-difluoro-4-[(1-hydroxycyclobutyl)methoxy]phenyl}-4-{[(3S,5S)-5-fluoropiperidin-3-yl]amino}pyrido[3,2-d]pyrimidine-8-carboxamide: To a stirred mixture of tert-butyl (3S,5S)-3-[(8-carbamoyl-6-{3,5-difluoro-4-[(1-hydroxycyclobutyl)methoxy]phenyl}pyrido[3,2-d]pyrimidin-4-yl)amino]-5-fluoropiperidine-1-carboxylate (180 mg; 0.20 mmol) in DCM (3 ml) was added TFA (1 ml) dropwise at 0 degrees C. The resulting mixture was stirred for 1 h at room temperature under nitrogen atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 20% B to 50% B in 8 min, 50% B; Wave Length: 254 nm; RT1 (min): 7; Number Of Runs: 4) to afford 6-{3,5-difluoro-4-[(1-hydroxycyclobutyl)methoxy]phenyl}-4-{[(3S,5S)-5-fluoropiperidin-3-yl]amino}pyrido[3,2-d]pyrimidine-8-carboxamide as yellow solid (93.90 mg; 85.8%).

HPLC 93% purity, 3.35 min. MS:m/z=503.00 [M+H]^{+ 1}H NMR (DMSO, 400 MHz): δ 9.93 (d, 1H, J=3.6 Hz), 8.82 (s, 1H), 8.58 (s, 1H), 8.50 (d, 1H, J=8.8 Hz), 8.24 (m, 3H), 5.27 (s, 1H), 4.89 (d, 1H, J=47.9 Hz), 4.63 (dt, 1H, J=10.1, 5.1, 5.1 Hz), 4.16 (s, 2H), 2.89 (m, 4H), 2.61 (s, 1H), 2.12 (m, 6H), 1.63 (m, 2H) ppm

### Example 34: 4-(((3S,5S)-5-fluoropiperidin-3-yl)amino)-6-(4-((1-hydroxycyclopropyl)methoxy)phenyl)pyrido[3,2-d]pyrimidine-8-carboxamide

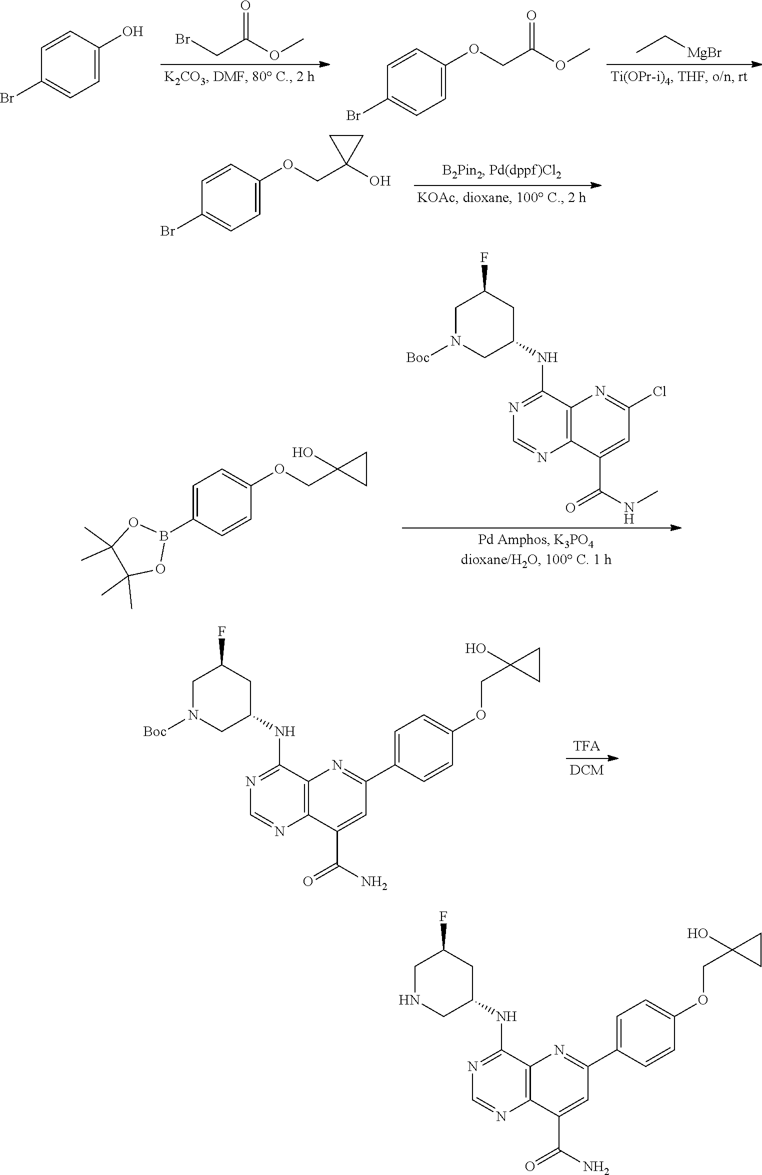

methyl 2-(4-bromophenoxy)acetate: To a stirred solution 4-bromophenol (9.50 g; 52.17 mmol) and methyl 2-bromoacetate (10.08 g; 62.60 mmol) in DMF (40 ml) were added potassium carbonate (11.38 g; 78.25 mmol) in portion at room temperature under nitrogen atmosphere. The resulting mixture was stirred for overnight at 80 degrees C. under nitrogen atmosphere. The reaction was added 500 ml water. The mixture was extracted with EA (3×250 mL). The combined organic layers were washed with brine, dried over anhydrous Na₂SO₄. After filtration, the filtrate was concentrated under reduced pressure to afford methyl 2-(4-bromophenoxy)acetate as brown liquid (11.32 g; Crude Product).

1-[(4-bromophenoxy)methyl]cyclopropan-1-ol: To a stirred mixture of methyl 2-(4-bromophenoxy)acetate (11.32 g; 45.68 mmol) in THF (110.00 ml) was added Ti(Oi-Pr)₄ (13.18 g; 44.05 mmol) and bromo(ethyl)magnesium (41.76 ml; 125.28 mmol) dropwise at 0 degrees C. The resulting mixture was stirred for overnight at room temperature under nitrogen atmosphere. The reaction was quenched with water at 0 degrees C. The resulting mixture was filtered; the filter cake was washed with EA (3×20 ml). The filtrate was extracted with EA (3×60 mL). The combined organic layers were washed with brine, dried over anhydrous Na₂SO₄. After filtration, the filtrate was concentrated under reduced pressure. The residue was purified by silica gel column chromatography, eluted with 13% EA in PE to afford 1-[(4-bromophenoxy)methyl]cyclopropan-1-ol as white solid (4.67 g; 40.3%).

1-{[4-(4,4,5,5-tetramethyl-1,3,2-dioxaborolan-2-yl)phenoxy]methyl}cyclopropan-1-ol: To a stirred mixture of 1-[(4-bromophenoxy)methyl]cyclopropan-1-ol (4.67 g; 18.40 mmol) and BPD (9.77 g; 36.55 mmol) in Dioxane-1,4 (30 ml) was added KOAc (5.68 g; 54.98 mmol) and Pd(dppf)Cl₂ (1.42 g; 1.84 mmol) in portion at room temperature. The resulting mixture was stirred for 2 h at 100 degrees C. under nitrogen atmosphere. The resulting mixture was concentrated under reduced pressure. The residue was purified by silica gel column chromatography, eluted with 15% EA in PE. The mixture was purified by reverse flash chromatography with the following conditions: column, C18 silica gel; mobile phase, ACN in water, 45% to 50% gradient in 10 min; detector, UV 254 nm to afford 1-{[4-(4,4,5,5-tetramethyl-1,3,2-dioxaborolan-2-yl)phenoxy]methyl}cyclopropan-1-ol as white solid (1.70 g; 26.4%).

tert-butyl (3S,5S)-3-[(8-carbamoyl-6-{4-[(1-hydroxycyclopropyl)methoxy]phenyl}pyrido[3,2-d]pyrimidin-4-yl)amino]-5-fluoropiperidine-1-carboxylate: To a stirred solution of tert-butyl (3S,5S)-3-({8-carbamoyl-6-chloropyrido[3,2-d]pyrimidin-4-yl}amino)-5-fluoropiperidine-1-carboxylate (300 mg; 0.55 mmol) and 1-{[4-(4,4,5,5-tetramethyl-1,3,2-dioxaborolan-2-yl)phenoxy]methyl}cyclopropan-1-ol (513 mg; 1.47 mmol) in Dioxane-1,4 (5 ml) and Water (0.5 ml) were added K₃PO₄ (600 mg; 2.69 mmol) and Pd(dppf)Cl₂ (47 mg; 0.07 mmol) in portion at room temperature under N₂ atmosphere. The resulting mixture was stirred for 1 h at 80-degree C. under N₂ atmosphere. The resulting mixture was concentrated under reduced pressure. The residue was purified by silica gel column chromatography, eluted with 80% EA in PE to afford tert-butyl (3S,5S)-3-[(8-carbamoyl-6-{4-[(1-hydroxycyclopropyl)methoxy]phenyl}pyrido[3,2-d]pyrimidin-4-yl)amino]-5-fluoropiperidine-1-carboxylate as yellow solid (200 mg; 36.4%).

4-{[(3S,5S)-5-fluoropiperidin-3-yl]amino}-6-{4-[(1-hydroxycyclopropyl)methoxy]phenyl}pyrido[3,2-d]pyrimidine-8-carboxamide: To a stirred mixture of tert-butyl (3S,5S)-3-[(8-carbamoyl-6-{4-[(1-hydroxycyclopropyl)methoxy]phenyl}pyrido[3,2-d]pyrimidin-4-yl)amino]-5-fluoropiperidine-1-carboxylate (180 mg; 0.18 mmol) in DCM (3 ml) was added TFA (1 ml) dropwise at 0 degrees C. The resulting mixture was stirred for 1 h at room temperature under nitrogen atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 18% B to 44% B in 9 min, 44% B; Wave Length: 254 nm; RT1 (min): 7; Number Of Runs: 3) to afford 4-{[(3S,5S)-5-fluoropiperidin-3-yl]amino}-6-{4-[(1-hydroxycyclopropyl)methoxy]phenyl}pyrido[3,2-d]pyrimidine-8-carboxamide as yellow solid (42.7 mg; 50.1%).

HPLC 96% purity, 6.13 min. MS:m/z=453.05 [M+H]⁺.

1H NMR (400 MHz, DMSO, 24° C.) 5 0.56-0.81 (m, 4H), 1.96-2.39 (m, 2H), 2.51 (s, 1H), 2.65-3.17 (m, 4H), 4.07 (s, 2H), 4.60 (dq, J=14.0, 9.3, 7.2 Hz, 1H), 4.87 (d, J=48.1 Hz, 1H), 5.64 (s, 1H), 7.06-7.21 (m, 2H), 8.21 (d, J=3.8 Hz, 1H), 8.28-8.49 (m, 3H), 8.55 (s, 1H), 8.80 (s, 1H), 10.05 (d, J=3.7 Hz, 1H).

### Example 35: 4-(((3S,5S)-5-fluoropiperidin-3-yl)amino)-6-(4-((1-hydroxycyclopentyl)methoxy)phenyl)pyrido[3,2-d]pyrimidine-8-carboxamide

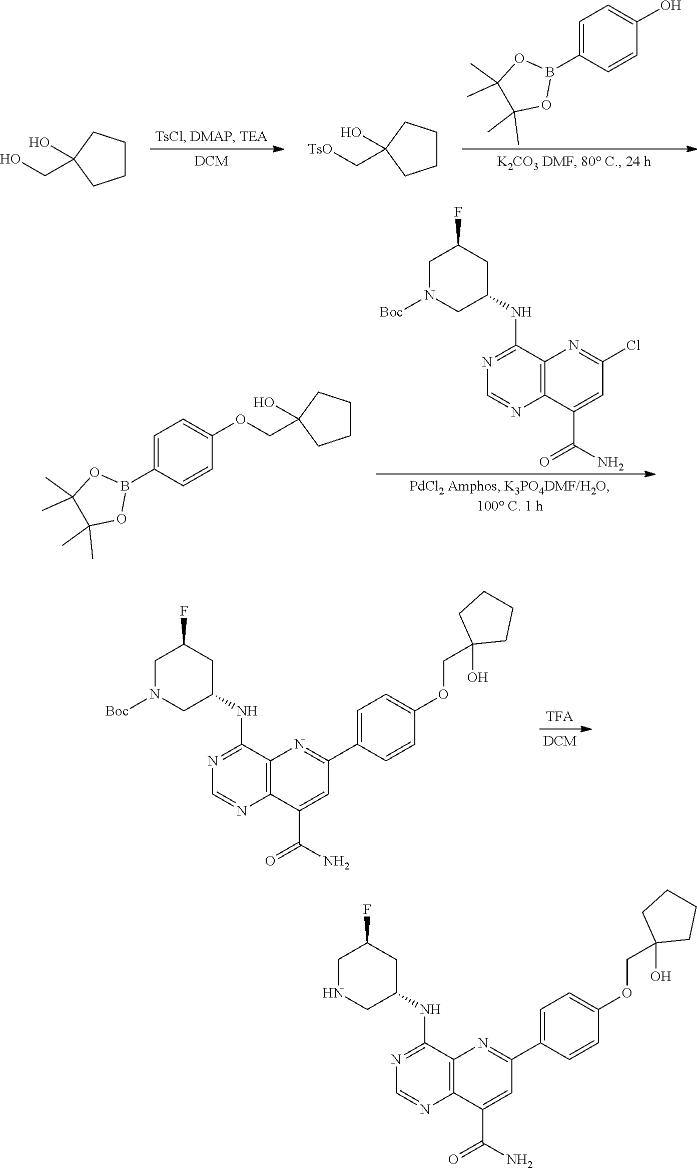

(1-hydroxycyclopentyl)methyl 4-methylbenzene-1-sulfonate: To a stirred mixture of 1-(hydroxymethyl)cyclopentan-1-ol (500.00 mg; 4.09 mmol) and TsCI (1230.97 mg; 6.13 mmol) in DCM (15.00 ml) was added DMAP (55.00 mg; 0.41 mmol) dropwise at 25 degrees C. The resulting mixture was stirred for 12 h at 25 degrees C. under nitrogen atmosphere. The resulting mixture was concentrated under vacuum. The residue was purified by silica gel column chromatography, eluted with PE:EA=8:2 to afford (1-hydroxycyclopentyl)methyl 4-methylbenzene-1-sulfonate as yellow oil (800 mg; 72.4%).

1-{[4-(4,4,5,5-tetramethyl-1,3,2-dioxaborolan-2-yl)phenoxy]methyl}cyclopentan-1-ol: To a stirred mixture of (1-hydroxycyclopentyl)methyl 4-methylbenzene-1-sulfonate (492 mg; 1.55 mmol) and 4-(4,4,5,5-tetramethyl-1,3,2-dioxaborolan-2-yl)phenol (300 mg; 1.30 mmol) in DMF (15 ml) was added K₂CO₃ (565 mg; 3.88 mmol) in portion at 25 degrees C. The resulting mixture was stirred for 12 h at 25 degrees C. under nitrogen atmosphere. The resulting mixture was filtered, The filtrate was concentrated under reduced pressure and to afford 1-{[4-(4,4,5,5-tetramethyl-1,3,2-dioxaborolan-2-yl)phenoxy]methyl}cyclopentan-1-ol as yellow oil (300 mg; Crude Product).

tert-butyl (3S,5S)-3-[(8-carbamoyl-6-{4-[(1-hydroxycyclopentyl)methoxy]phenyl}pyrido[3,2-d]pyrimidin-4-yl)amino]-5-fluoropiperidine-1-carboxylate: To a stirred mixture of 1-{[4-(4,4,5,5-tetramethyl-1,3,2-dioxaborolan-2-yl)phenoxy]methyl}cyclopentan-1-ol (250 mg; 0.50 mmol) and tert-butyl (3S,5S)-3-({8-carbamoyl-6-chloropyrido[3,2-d]pyrimidin-4-yl}amino)-5-fluoropiperidine-1-carboxylate (270 mg; 0.50 mmol) in DMF (10 ml) were added Pd(AmPhos)Cl₂ (37 mg; 0.05 mmol), K₃PO₄ (333 mg; 1.49 mmol) and H₂O (2 ml) in portion at 25 degrees C. The resulting mixture was stirred for 2 h at 100 degrees C. under nitrogen atmosphere. The resulting mixture was concentrated under vacuum. The residue was purified by silica gel column chromatography, eluted with PE:EA=1:1 to afford tert-butyl (3S,5S)-3-[(8-carbamoyl-6-{4-[(1-hydroxycyclopentyl)methoxy]phenyl}pyrido[3,2-d]pyrimidin-4-yl)amino]-5-fluoropiperidine-1-carboxylate as yellow solid (200 mg; 55.6%).

4-{[(3S,5S)-5-fluoropiperidin-3-yl]amino}-6-{4-[(1-hydroxycyclopentyl)methoxy]phenyl}pyrido[3,2-d]pyrimidine-8-carboxamide: To a stirred mixture of tert-butyl (3S,5S)-3-[(8-carbamoyl-6-{4-[(1-hydroxycyclopentyl)methoxy]phenyl}pyrido[3,2-d]pyrimidin-4-yl)amino]-5-fluoropiperidine-1-carboxylate (180 mg; 0.25 mmol) in DCM (8 ml) was added TFA (2 ml) in portion at 25 degrees C. The resulting mixture was stirred for 2 h at 25 degrees C. under nitrogen atmosphere. The resulting mixture was concentrated under vacuum. The crude product was purified by Prep-HPLC with the following conditions Column: YMC-Actus Triart C18 ExRS, 20*250 mm, 5 μm; Mobile Phase A: Water (10 mmol/L NH₄HCO₃+0.05% NH₄OH), Mobile Phase B: ACN; Flow rate: 60 mL/min; Gradient: 25% B to 55% B in 8 min, 55% B; Wave Length: 254 nm; RT1 (min): 7; Number Of Runs: 2) to afford 4-{[(3S,5S)-5-fluoropiperidin-3-yl]amino}-6-{4-[(1-hydroxycyclopentyl)methoxy]phenyl}pyrido[3,2-d]pyrimidine-8-carboxamide as yellow solid (64.40 mg; 53.2%).

HPLC 98% purity, 2.98 min. MS:m/z=481.05 [M+H]⁺.

¹H NMR (400 MHz, DMSO, 23° C.) δ 1.56-1.84 (m, 8H), 2.06-2.27 (m, 2H), 2.56-2.86 (m, 3H), 2.94-3.14 (m, 2H), 3.97 (s, 2H), 4.61 (d, J=11.9 Hz, 2H), 4.87 (d, J=47.9 Hz, 1H), 7.11 (d, J=8.5 Hz, 2H), 8.23 (d, J=3.8 Hz, 1H), 8.35 (dd, J=8.5, 5.5 Hz, 3H), 8.56 (s, 1H), 8.81 (s, 1H), 10.06 (d, J=3.7 Hz, 1H)

### Example 36: 6-(1-cyclopropyl-1H-pyrazol-4-yl)-4-(((3S,5S)-5-fluoropiperidin-3-yl)amino)pyrido[3,2-d]pyrimidine-8-carboxamide

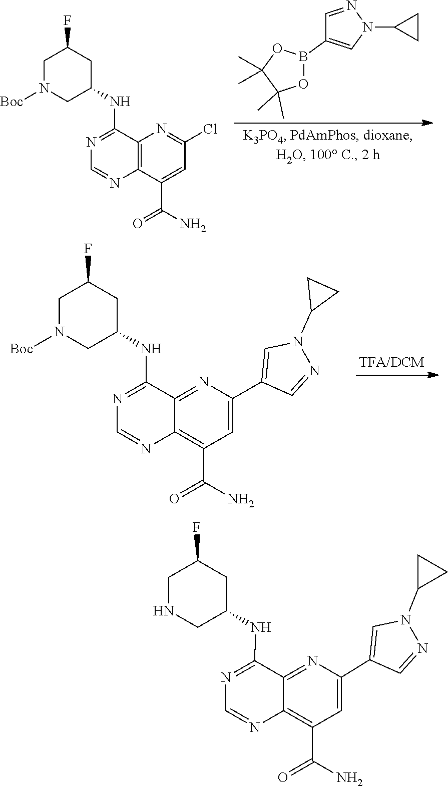

tert-butyl (3S,5S)-3-{[8-carbamoyl-6-(1-cyclopropyl-1H-pyrazol-4-yl)pyrido[3,2-d]pyrimidin-4-yl]amino}-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (120 mg; 0.22 mmol) and 1-cyclopropyl-4-(4,4,5,5-tetramethyl-1,3,2-dioxaborolan-2-yl)-1H-pyrazole (63.33 mg; 0.27 mmol) in Dioxane-1,4 (10 ml) and H₂O (2 ml) were added Pd(AmPhos)Cl₂ (16.47 mg; 0.02 mmol) and K₃PO₄ (148.09 mg; 0.66 mmol) at room temperature. After stirring for 2 h at 100 degrees C. under argon atmosphere. The mixture was concentrated under reduced pressure. The residue was purified by silica gel column chromatography, eluted with CH₂Cl₂/MeOH (12:1) to afford tert-butyl (3S,5S)-3-{[8-carbamoyl-6-(1-cyclopropyl-1H-pyrazol-4-yl)pyrido[3,2-d]pyrimidin-4-yl]amino}-5-fluoropiperidine-1-carboxylate as yellow solid (110 mg; 90.38%).

6-(1-cyclopropyl-1H-pyrazol-4-yl)-4-{[(3S,5S)-5-fluoropiperidin-3-yl]amino}pyrido[3,2-d]pyrimidine-8-carboxamide: To a stirred mixture of tert-butyl (3S,5S)-3-{[8-carbamoyl-6-(1-cyclopropyl-1H-pyrazol-4-yl)pyrido[3,2-d]pyrimidin-4-yl]amino}-5-fluoropiperidine-1-carboxylate (140.00 mg; 0.25 mmol) and TFA (1.00 ml) in DCM (6.00 ml) at room temperature. After stirring for 1 h at room temperature. The mixture was concentrated under reduced pressure. The crude product was purified by Prep-HPLC(Column: YMC-Actus Triart C18 ExRS, 20*250 mm, 5 μm; Mobile Phase A: Water (10 mmol/L NH₄HCO₃+0.05% NH₄OH), Mobile Phase B: ACN; Flow rate: 60 mL/min; Gradient: 15% B to 45% B in 8 min, 45% B; Wave Length: 254 nm; RT1 (min): 7; Number Of Runs: 2) to afford 6-(1-cyclopropyl-1H-pyrazol-4-yl)-4-{[(3S,5S)-5-fluoropiperidin-3-yl]amino}pyrido[3,2-d]pyrimidine-8-carboxamide as yellow solid (38.5 mg; 38.06%).

HPLC 99% purity, 2.16 min. MS:m/z=397.20 [M+H]⁺.

¹H NMR (400 MHz, DMSO-d6): 10.01 (d, J=3.7 Hz, 1H), 8.78 (s, 1H), 8.55 (d, J=17.5 Hz, 2H), 8.41 (s, 1H), 8.23-8.16 (m, 2H), 4.94-4.82 (s, 1H), 4.58 (dd, J=13.2, 7.6 Hz, 1H), 3.83 (tt, J=7.5, 3.9 Hz, 1H), 3.05 (ddd, J=25.9, 13.3, 7.6 Hz, 2H), 2.71 (d, J=12.3 Hz, 2H), 2.26-2.12 (m, 2H), 1.20-1.12 (m, 2H), 1.12-0.99 (m, 2H).

### Example 37: 6-(3,5-difluoro-4-((4-hydroxytetrahydro-2H-pyran-4-yl)methoxy)phenyl)-4-(((3S,5S)-5-fluoropiperidin-3-yl)amino)pyrido[3,2-d]pyrimidine-8-carboxamide

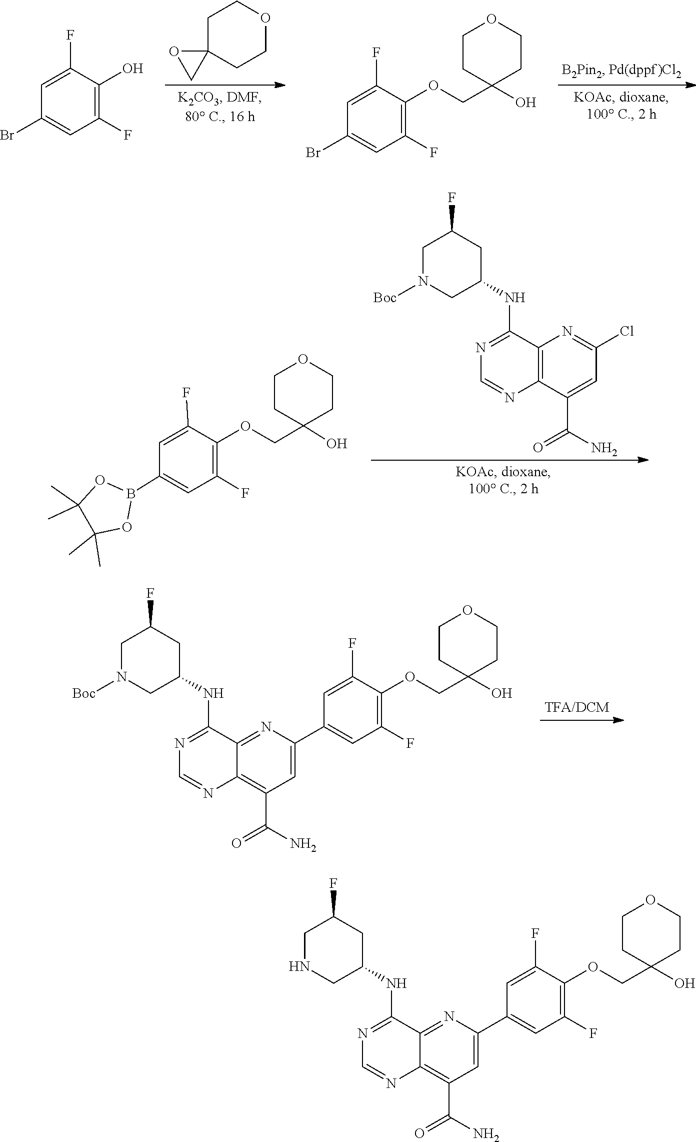

4-[(4-bromo-2,6-difluorophenoxy)methyl]oxan-4-ol: To a stirred mixture of 4-bromo-2,6-difluorophenol (1.00 g; 4.55 mmol) and 1,6-dioxaspiro[2.5]octane (1.64 g; 13.64 mmol) in DMF (15.00 ml) was added K₂CO₃ (1.98 g; 13.64 mmol) at room temperature. After stirring for 24 h at 80 degrees C. The resulting mixture was extracted with EtOAc and quenched with water. The combined organic layers were washed with brine, dried over anhydrous Na₂SO₄. After filtration, the filtrate was concentrated under reduced pressure. The crude product was purified by silica gel column chromatography, eluted with PE/EtOAc (7:3) to afford 4-[(4-bromo-2,6-difluorophenoxy)methyl]oxan-4-ol as light yellow oil (1.45 g; 33.94%).

4-{[2,6-difluoro-4-(4,4,5,5-tetramethyl-1,3,2-dioxaborolan-2-yl)phenoxy]methyl}oxan-4-ol: To a stirred mixture of 4-[(4-bromo-2,6-difluorophenoxy)methyl]oxan-4-ol (1.40 g; 1.51 mmol) and BPD (1.21 g; 4.53 mmol) in Dioxane-1,4 (20.00 ml) were added KOAc (0.49 g; 4.53 mmol) and Pd(dppf)Cl₂ (0.12 g; 0.15 mmol) at room temperature. After stirring for 3 h at 100 degrees C. under argon atmosphere. The resulting mixture was concentrated under reduced pressure and purified by silica gel column chromatography, eluted with PE/EtOAc (3:2) to afford 4-{[2,6-difluoro-4-(4,4,5,5-tetramethyl-1,3,2-dioxaborolan-2-yl)phenoxy]methyl}oxan-4-ol as yellow solid (750 mg; 99.47%).

tert-butyl (3S,5S)-3-[(8-carbamoyl-6-{3,5-difluoro-4-[(4-hydroxyoxan-4-yl)methoxy]phenyl}pyrido[3,2-d]pyrimidin-4-yl)amino]-5-fluoropiperidine-1-carboxylate: To a stirred mixture of tert-butyl (3S,5S)-3-({8-carbamoyl-6-chloropyrido[3,2-d]pyrimidin-4-yl}amino)-5-fluoropiperidine-1-carboxylate (130.00 mg; 0.24 mmol) and 4-{[2,6-difluoro-4-(4,4,5,5-tetramethyl-1,3,2-dioxaborolan-2-yl)phenoxy]methyl}oxan-4-ol (155.20 mg; 0.31 mmol) in Dioxane-1,4 (5.00 ml) and water (1.00 ml) were added K₃PO₄ (160.39 mg; 0.72 mmol) and Bis(di-tert-butyl(4-dimethylaminophenyl)phosphine)dichloropalladium(II) (17.84 mg; 0.02 mmol) at room temperature. After stirring for 2 h at 100 degrees C. under argon atmosphere. The resulting mixture was concentrated under reduced pressure and purified by silica gel column chromatography, eluted with CH₂Cl₂/MeOH (7:3) to afford tert-butyl (3S,5S)-3-[(8-carbamoyl-6-{3,5-difluoro-4-[(4-hydroxyoxan-4-yl)methoxy]phenyl}pyrido[3,2-d]pyrimidin-4-yl)amino]-5-fluoropiperidine-1-carboxylate as yellow solid (125.00 mg; 68.07%).

6-{3,5-difluoro-4-[(4-hydroxyoxan-4-yl)methoxy]phenyl}-4-{[(3S,5S)-5-fluoropiperidin-3-yl]amino}pyrido[3,2-d]pyrimidine-8-carboxamide: To a solution of tert-butyl (3S,5S)-3-[(8-carbamoyl-6-{3,5-difluoro-4-[(4-hydroxyoxan-4-yl)methoxy]phenyl}pyrido[3,2-d]pyrimidin-4-yl)amino]-5-fluoropiperidine-1-carboxylate (120.00 mg; 0.16 mmol) in DCM (3.00 ml) were added TFA (1.00 ml) at room temperature. After stirring for 1 h at room temperature. The resulting mixture was concentrated under reduced pressure and purified by Prep-HPLC(Column: YMC-Actus Triart C18 ExRS, 30*150 mm, 5 μm; Mobile Phase A: Water (10 mmol/L NH₄HCO₃+0.05% NH₄OH), Mobile Phase B: ACN; Flow rate: 60 mL/min; Gradient: 20% B to 50% B in 8 min, 50% B; Wave Length: 254 nm; RT1 (min): 7; Number Of Runs: 3) to afford 6-{3,5-difluoro-4-[(4-hydroxyoxan-4-yl)methoxy]phenyl}-4-{[(3S,5S)-5-fluoropiperidin-3-yl]amino}pyrido[3,2-d]pyrimidine-8-carboxamide as yellow solid (63.3 mg; 74.58%).

HPLC 98% purity, 2.72 min. MS:m/z=533.25 [M+H]⁺.

¹H NMR (300 MHz, DMSO-d6): 9.90 (s, 1H), 8.82 (s, 1H), 8.57 (s, 1H), 8.49 (d, J=8.8 Hz, 1H), 8.32-8.19 (m, 3H), 4.94 (s, 1H), 4.76 (d, J=13.7 Hz, 1H), 4.60 (s, 1H), 4.01 (s, 2H), 3.65 (d, J=8.1 Hz, 4H), 3.02 (t, J=14.8 Hz, 2H), 2.71-2.57 (m, 2H), 2.23 (s, 2H), 1.77 (dt, J=16.2, 8.2 Hz, 2H), 1.47 (d, J=13.7 Hz, 2H).

### Example 38: 4-(((3R,4S)-4-fluoropiperidin-3-yl)amino)-6-(4-(2-hydroxy-2-methylpropoxy)phenyl)pyrido[3,2-d]pyrimidine-8-carboxamide

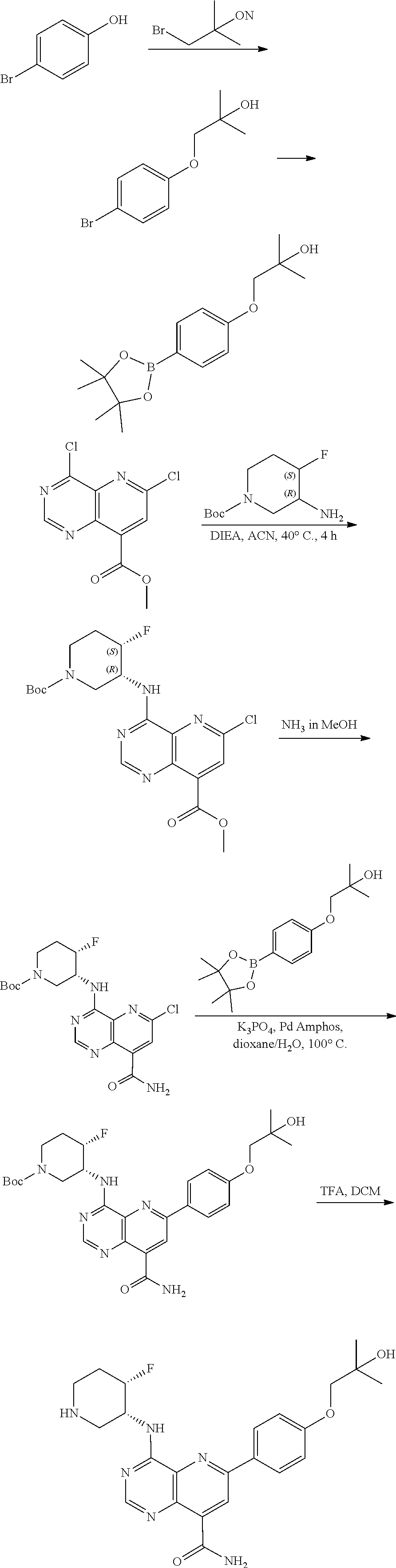

1-(4-bromophenoxy)-2-methylpropan-2-ol: To a solution of 4-bromophenol (10.00 g; 54.91 mmol.) and 2,2-dimethyloxirane (6.25 g; 82.34 mmol.) in DMF (100.00 ml) were added potassium carbonate (23.95 g; 164.63 mmol.) in portions at room temperature under nitrogen atmosphere. The resulting mixture was stirred for 3 h at 140 degrees C. under nitrogen atmosphere. The resulting mixture was added water (500 mL). The resulting mixture was extracted with EtOAc (3×300 mL). The combined organic layers were washed with brine (1×500 mL), dried over anhydrous Na₂SO₄. After filtration, the filtrate was concentrated under reduced pressure to afford 1-(4-bromophenoxy)-2-methylpropan-2-ol as a yellow oil (13.40 g, Crude Product).

2-methyl-1-[4-(4,4,5,5-tetramethyl-1,3,2-dioxaborolan-2-yl)phenoxy]propan-2-ol: To a stirred mixture of 1-(4-bromophenoxy)-2-methylpropan-2-ol (13.00 g; 51.31 mmol.) and BPD (27.43 g; 102.62 mmol.) in Dioxane-1,4 (130.00 ml) was added KOAc (15.90 g; 153.92 mmol.) and PCl₂ (3.95 g; 5.13 mmol.) in portions at room temperature. The resulting mixture was stirred for 1 h at 100 degrees C. under nitrogen atmosphere. The resulting mixture was concentrated under reduced pressure. The residue was purified by silica gel column chromatography, eluted with 8% EA in PE to afford 2-methyl-1-[4-(4,4,5,5-tetramethyl-1,3,2-dioxaborolan-2-yl)phenoxy]propan-2-ol as a yellow solid (9.32 g; 59.0%).

tert-butyl (3R,4S)-3-{[6-chloro-8-(methoxycarbonyl)pyrido[3,2-d]pyrimidin-4-yl]amino}-4-fluoropiperidine-1-carboxylate: To a stirred mixture of methyl 4,6-dichloropyrido[3,2-d]pyrimidine-8-carboxylate (520 mg; 1.61 mmol.) and tert-butyl (3R,4S)-3-amino-4-fluoropiperidine-1-carboxylate (488.00 mg; 2.12 mmol.) in MeCN (7 ml) was added DIEA (1.06 ml; 5.79 mmol.) dropwise at room temperature. The resulting mixture was stirred for 1 h at 40 degrees C. under nitrogen atmosphere. The resulting mixture was added 25 ml water then the mixture was extracted with EA (3×15 mL). The combined organic layers were washed with brine, dried over anhydrous Na₂SO₄. After filtration, the filtrate was concentrated under reduced pressure. The residue was purified by silica gel column chromatography, eluted with 20% EA in PE to afford tert-butyl (3R,4S)-3-{[6-chloro-8-(methoxycarbonyl)pyrido[3,2-d]pyrimidin-4-yl]amino}-4-fluoropiperidine-1-carboxylate as a yellow solid. (600 mg; 54.4%).

tert-butyl (3R,4S)-3-({8-carbamoyl-6-chloropyrido[3,2-d]pyrimidin-4-yl}amino)-4-fluoropiperidine-1-carboxylate: A stirred mixture of tert-butyl (3R,4S)-3-{[6-chloro-8-(methoxycarbonyl)pyrido[3,2-d]pyrimidin-4-yl]amino}-4-fluoropiperidine-1-carboxylate (580 mg; 0.85 mmol.) in NH₃ in MeOH (10 ml) was stirred for 1 h at 40 degrees C. The resulting mixture was concentrated under reduced pressure to afford tert-butyl (3R,4S)-3-({8-carbamoyl-6-chloropyrido[3,2-d]pyrimidin-4-yl}amino)-4-fluoropiperidine-1-carboxylate as an orange yellow solid (440 mg; Crude Product).

tert-butyl (3R,4S)-3-({8-carbamoyl-6-[4-(2-hydroxy-2-methylpropoxy)phenyl]pyrido[3,2-d]pyrimidin-4-yl}amino)-4-fluoropiperidine-1-carboxylate: To a stirred solution of tert-butyl (3R,4S)-3-({8-carbamoyl-6-chloropyrido[3,2-d]pyrimidin-4-yl}amino)-4-fluoropiperidine-1-carboxylate (150 mg; 0.28 mmol) and 2-methyl-1-[4-(4,4,5,5-tetramethyl-1,3,2-dioxaborolan-2-yl)phenoxy]propan-2-ol (124 mg; 0.40 mmol.) in Dioxane-1,4 (4 ml) and Water (0.40 ml) were added PCl₂ (26 mg; 0.03 mmol.) and potassium phosphate tribasic (225 mg; 1.01 mmol.) in portions at room temperature under N₂ atmosphere. The resulting mixture was stirred for 2 h at 100 degrees C. under N₂ atmosphere. The resulting mixture was diluted with water (15 mL), extracted with EtOAc (3×10 mL). The combined organic layers were washed with brine (15 ml), dried over anhydrous Na₂SO₄. The resulting mixture was concentrated under reduced pressure. The residue was purified by silica gel column, eluted with 75% EtOAc in PE to afford tert-butyl (3R,4S)-3-({8-carbamoyl-6-[4-(2-hydroxy-2-methylpropoxy)phenyl]pyrido[3,2-d]pyrimidin-4-yl}amino)-4-fluoropiperidine-1-carboxylate as a yellow solid (140.00 mg 84.06%).

4-{[(3R,4S)-4-fluoropiperidin-3-yl]amino}-6-[4-(2-hydroxy-2-methylpropoxy)phenyl]pyrido[3,2-d]pyrimidine-8-carboxamide: To a stirred solution of tert-butyl (3R,4S)-3-({8-carbamoyl-6-[4-(2-hydroxy-2-methylpropoxy)phenyl]pyrido[3,2-d]pyrimidin-4-yl}amino)-4-fluoropiperidine-1-carboxylate (120 mg; 0.20 mmol.) in Dichloromethane (3 ml) was added TFA (1 ml) dropwise at 0 degree C. under N₂ atmosphere. The resulting mixture was stirred for 30 min at room temperature under N₂ atmosphere. The resulting mixture was concentrated under reduced pressure. The residue was purified by Prep-HPLC with following condition: Column:Column: XBridge Prep OBD C18 Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 20% B to 50% B in 9 min, 50% B; Wave Length: 254 nm; RT1 (min): 7; Number Of Runs: 2) to afford 4-{[(3R,4S)-4-fluoropiperidin-3-yl]amino}-6-[4-(2-hydroxy-2-methylpropoxy)phenyl]pyrido[3,2-d]pyrimidine-8-carboxamide as a yellow solid (56.70 mg; 60.22%).

HPLC 98%, 2.90 min. M/S: m/z=455.15. ¹H-NMR (300 MHz, DMSO-d6) 9.98 (d, J=3.8 Hz, 1H), 8.82 (s, 1H), 8.60 (s, 1H), 8.27 (d, J=8.4 Hz, 3H), 8.11 (d, J=8.7 Hz, 1H), 7.14 (d, J=8.4 Hz, 2H), 5.10 (d, J=50.5 Hz, 1H), 4.62 (d, J=53.1 Hz, 2H), 3.84 (s, 2H), 3.35 (s, 1H), 2.98 (s, 2H), 2.78 (d, J=16.3 Hz, 2H), 2.00-1.89 (m, 2H), 1.25 (s, 6H).

### Example 39: (S)-6-(3,5-difluoro-4-((1-hydroxycyclobutyl)methoxy)phenyl)-4-(piperidin-3-ylamino)pyrido[3,2-d]pyrimidine-8-carboxamide

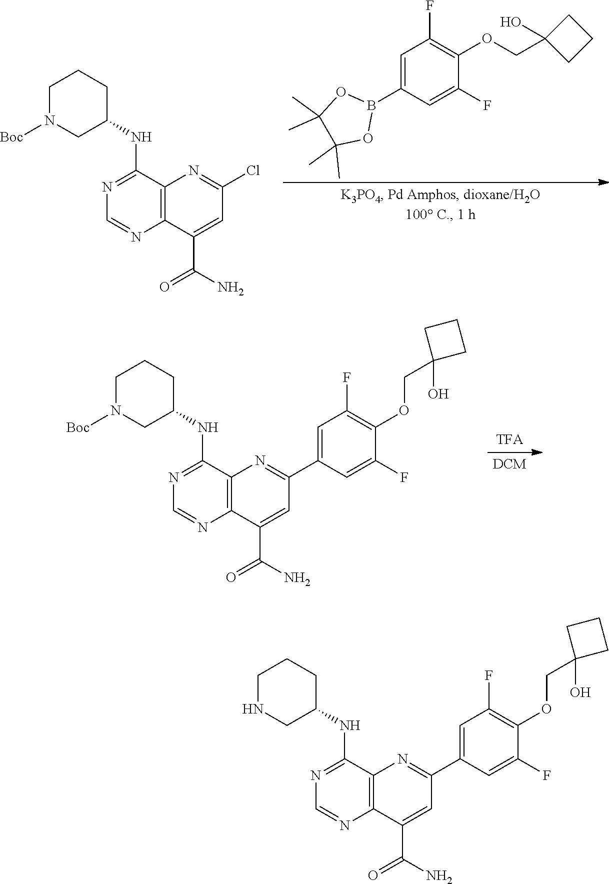 [SMILES: CC(C)(C)OC(=O)N1CCC[C@H](Nc2ncnc3c(C(N)=O)cc(Cl)nc23)C1 ; CC1(C)OB(c2cc(F)c(OCC3(O)CCC3)c(F)c2)OC1(C)C ; CC(C)(C)OC(=O)N1CCC[C@H](Nc2ncnc3c(C(N)=O)cc(-c4cc(F)c(OCC5(O)CCC5)c(F)c4)nc23)C1 ; NC(=O)c1cc(-c2cc(F)c(OCC3(O)CCC3)c(F)c2)nc2c(N[C@H]3CCCNC3)ncnc12]

tert-butyl (3S)-3-[(8-carbamoyl-6-{3,5-difluoro-4-[(1-hydroxycyclobutyl)methoxy]phenyl}pyrido[3,2-d]pyrimidin-4-yl)amino]piperidine-1-carboxylate: To a stirred mixture of tert-butyl (3S)-3-({8-carbamoyl-6-chloropyrido[3,2-d]pyrimidin-4-yl}amino)piperidine-1-carboxylate (150 mg; 0.30 mmol.) and 1-{[2,6-difluoro-4-(4,4,5,5-tetramethyl-1,3,2-dioxaborolan-2-yl)phenoxy]methyl}cyclobutan-1-ol (157 mg; 0.29 mmol.) in H₂O (3 ml) and 1,4-dioxane (10 ml) was added K₃PO₄ (235 mg; 1.05 mmol.) and PCl₂ (26 mg; 0.03 mmol) in portions at room temperature. The resulting mixture was stirred for 1 h at 100 degrees C. under nitrogen atmosphere. The resulting mixture was concentrated under reduced pressure. The residue was purified by silica gel column chromatography, eluted with 40% EA in PE to afford tert-butyl (3S)-3-[(8-carbamoyl-6-{3,5-difluoro-4-[(1-hydroxycyclobutyl)methoxy]phenyl}pyrido[3,2-d]pyrimidin-4-yl)amino]piperidine-1-carboxylate as a yellow solid (190 mg; 99.0%).

6-{3,5-difluoro-4-[(1-hydroxycyclobutyl)methoxy]phenyl}-4-{[(3S)-piperidin-3-yl]amino}pyrido[3,2-d]pyrimidine-8-carboxamide: To a stirred mixture of tert-butyl (3S)-3-[(8-carbamoyl-6-{3,5-difluoro-4-[(1-hydroxycyclobutyl)methoxy]phenyl}pyrido[3,2-d]pyrimidin-4-yl)amino]piperidine-1-carboxylate (170 mg; 0.27 mmol.) in DCM (3 ml) was added TFA (1 ml) dropwise at 0 degrees C. The resulting mixture was stirred for 1 h at room temperature under nitrogen atmosphere. The resulting mixture was concentrated under reduced pressure. The crude product was purified by Prep-HPLC with the following conditions (Column: Xselect CSH OBD Column 30*150 mm 5 um; Mobile Phase A: Water (10 mmol/L NH₄HCO₃+0.05% NH₄OH), Mobile Phase B: ACN; Flow rate: 60 mL/min; Gradient: 23% B to 53% B in 10 min, 53% B; Wave Length: 254 nm; RT1 (min): 7.58; Number Of Runs: 2) to afford 6-{3,5-difluoro-4-[(1-hydroxycyclobutyl)methoxy]phenyl}-4-{[(3S)-piperidin-3-yl]amino}pyrido[3,2-d]pyrimidine-8-carboxamide as a yellow solid (79.70 mg, 59.0%).

HPLC: 95%, 3.43 min. M/S: m/z=455.15. ¹H NMR (400 MHz, DMSO-d6) 9.95 (d, J=3.6 Hz, 1H), 8.80 (s, 1H), 8.56 (s, 1H), 8.48 (d, J=8.6 Hz, 1H), 8.28-8.15 (m, 3H), 5.28 (s, 1H), 4.31 (qt, J=9.1, 3.9 Hz, 1H), 4.15 (s, 2H), 3.08 (dd, J=11.9, 4.0 Hz, 1H), 2.88 (dt, J=12.6, 3.7 Hz, 1H), 2.74 (dd, J=11.8, 9.4 Hz, 1H), 2.56 (d, J=11.2 Hz, 1H), 2.51 (s, 1H), 2.17 (tt, J=8.9, 3.1 Hz, 2H), 2.12-1.92 (m, 3H), 1.91-1.78 (m, 1H), 1.71 (qd, J=6.7, 4.7, 3.2 Hz, 2H), 1.56 (dd, J=11.3, 8.8 Hz, 2H).

### Example 40: 6-(4-(2-cyano-2-hydroxypropoxy)phenyl)-4-(((S)-piperidin-3-yl)amino)pyrido[3,2-d]pyrimidine-8-carboxamide

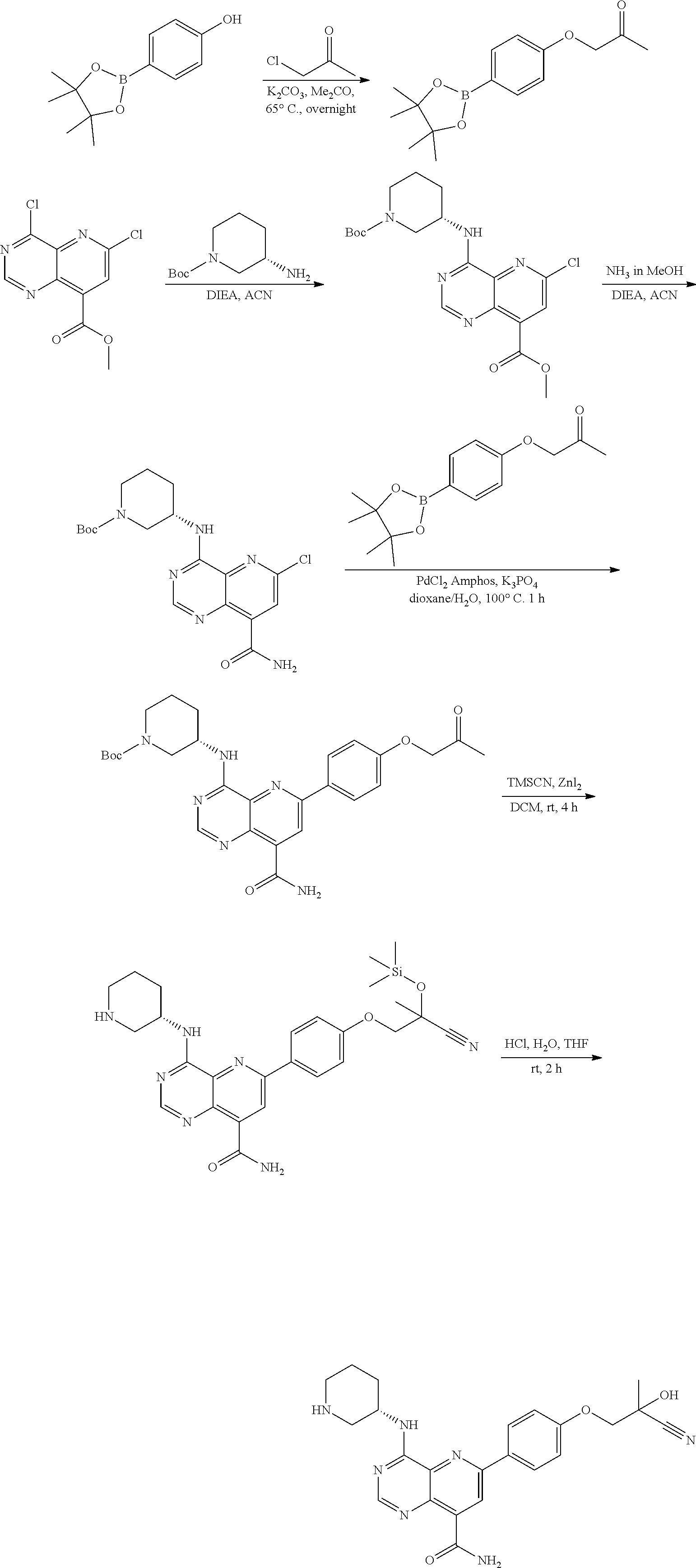

1-[4-(4,4,5,5-tetramethyl-1,3,2-dioxaborolan-2-yl)phenoxy]propan-2-one: To a stirred mixture of 4-(4,4,5,5-tetramethyl-1,3,2-dioxaborolan-2-yl)phenol (5 g; 21.58 mmol.) and 1-chloropropan-2-one (2.1 ml; 25.90 mmol.) in Acetone (40 ml) was added K₂CO₃ (9.42 g; 64.75 mmol.) in portions at 25 degrees C. The resulting mixture was stirred for 12 h at 60 degrees C. under nitrogen atmosphere. The reaction was quenched with H₂O at 25 degrees C. The resulting mixture was extracted with EA (3×50 mL). The combined organic layers were washed with brine, dried over anhydrous MgSO₄. After filtration, the filtrate was concentrated under reduced pressure. The residue was purified by C18 column chromatography, eluted with ACN:H₂O=80:20 to afford 1-[4-(4,4,5,5-tetramethyl-1,3,2-dioxaborolan-2-yl)phenoxy]propan-2-one as a yellow solid (2 g; 31.8%).

tert-butyl (3S)-3-{[6-chloro-8-(methoxycarbonyl)pyrido[3,2-d]pyrimidin-4-yl]amino}piperidine-1-carboxylate: To a mixture of methyl 4,6-dichloropyrido[3,2-d]pyrimidine-8-carboxylate (3.1 g; 9.87 mmol;) and tert-butyl (3S)-3-aminopiperidine-1-carboxylate (2.29 g; 10.86 mmol;) in ACN (30 ml) was added ethylbis(propan-2-yl)amine (4.03 g; 29.61 mmol;). After stirring for 2 h at 40 degrees C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (70:30) to afford tert-butyl (3S)-3-{[6-chloro-8-(methoxycarbonyl)pyrido[3,2-d]pyrimidin-4-yl]amino}piperidine-1-carboxylate as a yellow solid (2.8 g; 64.6%).

tert-butyl(3S)-3-({8-carbamoyl-6-chloropyrido[3,2-d]pyrimidin-4-yl}amino)piperidine-1-carboxylate: A mixture of tert-butyl (3S)-3-{[6-chloro-8-(methoxycarbonyl)pyrido[3,2-d]pyrimidin-4-yl]amino}piperidine-1-carboxylate (2.80 g; 6.37 mmol;) in NH₃ (g) in MeOH (20 ml) was stirred for 2 h at 40 degrees C. under nitrogen atmosphere. The resulting mixture was concentrated under reduced pressure. This resulted in tert-butyl (3S)-3-({8-carbamoyl-6-chloropyrido[3,2-d]pyrimidin-4-yl}amino)piperidine-1-carboxylate as a light yellow solid (2.30 g; 85.9%).

tert-butyl (3S)-3-({8-carbamoyl-6-[4-(2-oxopropoxy)phenyl]pyrido[3,2-d]pyrimidin-4-yl}amino)piperidine-1-carboxylate: To a stirred mixture of 1-[4-(4,4,5,5-tetramethyl-1,3,2-dioxaborolan-2-yl)phenoxy]propan-2-one (220 mg; 0.76 mmol.) and tert-butyl (3S)-3-({8-carbamoyl-6-chloropyrido[3,2-d]pyrimidin-4-yl}amino)piperidine-1-carboxylate (250 mg; 0.50 mmol.) in Dioxane-1,4 (8 ml) were added PCl₂ (37.41 mg; 0.05 mmol.), K₃PO₄ (336.45 mg; 1.51 mmol.) and H₂O (2 ml) in portions at 25 degrees C. The resulting mixture was stirred for 2 h at 100 degrees C. under nitrogen at morpheme. The resulting mixture was concentrated under vacuum. The residue was purified by silica gel column chromatography, eluted with PE:EA=4:6 to afford tert-butyl (3S)-3-({8-carbamoyl-6-[4-(2-oxopropoxy)phenyl]pyrido[3,2-d]pyrimidin-4-yl}amino)piperidine-1-carboxylate as a yellow solid (250 mg; 92.5%.).

6-(4-{2-cyano-2-methyl-2-[(trimethylsilyl)oxy]ethoxy}phenyl)-4-{[(3S)-piperidin-3-yl]amino}pyrido[3,2-d]pyrimidine-8-carboxamide: To a stirred mixture of tert-butyl (3S)-3-({8-carbamoyl-6-[4-(2-oxopropoxy)phenyl]pyrido[3,2-d]pyrimidin-4-yl}amino)piperidine-1-carboxylate (250 mg; 0.46 mmol.) and TMSCN (97 mg; 0.93 mmol.) in DCM (10 ml) was added ZnI₂ (156 mg; 0.46 mmol.) in portions at 25 degrees C. The resulting mixture was stirred for 12 h at 25 degrees C. under nitrogen atmosphere. The reaction was quenched with H₂O at room temperature. The resulting mixture was extracted with EA (3×100 mL). The combined organic layers were washed with brine, dried over anhydrous MgSO₄. After filtration, the filtrate was concentrated under reduced pressure to afford 6-(4-{2-cyano-2-methyl-2-[(trimethylsilyl)oxy]ethoxy}phenyl)-4-{[(3S)-piperidin-3-yl]amino}pyrido[3,2-d]pyrimidine-8-carboxamide as a yellow solid (200 mg; Crude Product).

6-[4-(2-cyano-2-hydroxy-2-methylethoxy)phenyl]-4-{[(3S)-piperidin-3-yl]amino}pyrido[3,2-d]pyrimidine-8-carboxamide hydrochloride: To a stirred mixture of 6-(4-{2-cyano-2-methyl-2-[(trimethylsilyl)oxy]ethoxy}phenyl)-4-{[(3S)-piperidin-3-yl]amino}pyrido[3,2-d]pyrimidine-8-carboxamide (200 mg; 0.30 mmol) in THF (5.00 ml) was added HCl (5 ml; 10.00 mmol.) dropwise at 25 degrees C. The resulting mixture was stirred for 2 h at 25 degrees C. under nitrogen atmosphere. The resulting mixture was concentrated under vacuum. The residue was purified by Column: Xselect CSH OBD Column 30*150 mm Sum; Mobile Phase A: Water (0.05% HCl), Mobile Phase B: ACN; Flow rate: 60 mL/min; Gradient: 5% B to 20% B in 10 min, 20% B; Wave Length: 254 nm; RT1 (min): 9.37; Number Of Runs: 2) to afford 6-[4-(2-cyano-2-hydroxy-2-methylethoxy)phenyl]-4-{[(3S)-piperidin-3-yl]amino}pyrido[3,2-d]pyrimidine-8-carboxamide hydrochloride as a yellow solid (52.50 mg; 36.0%).

HPLC: 99%, 2 0.89 min. M/S: m/z=448.2. ¹H NMR (400 MHz, DMSO-d6): 9.72 (s, 2H), 9.51 (s, 2H), 8.98 (d, J=3.1 Hz, 1H), 8.76 (d, J=1.9 Hz, 1H), 8.58 (dd, J=9.0, 3.0 Hz, 2H), 8.43 (s, 1H), 7.25-7.18 (m, 2H), 4.86 (s, 1H), 4.22 (q, J=9.8 Hz, 2H), 3.40 (d, J=11.0 Hz, 1H), 3.28 (d, J=14.1 Hz, 2H), 2.82 (d, J=11.4 Hz, 1H), 2.51 (p, J=1.9 Hz, 1H), 2.08 (s, 1H), 1.97 (s, 1H), 1.93 (td, J=26.4, 23.8, 10.9 Hz, 1H), 1.64 (s, 3H).

### Example 41: (S)-6-(4-((1-hydroxycyclopentyl)methoxy)phenyl)-4-(piperidin-3-ylamino)pyrido[3,2-d]pyrimidine-8-carboxamide

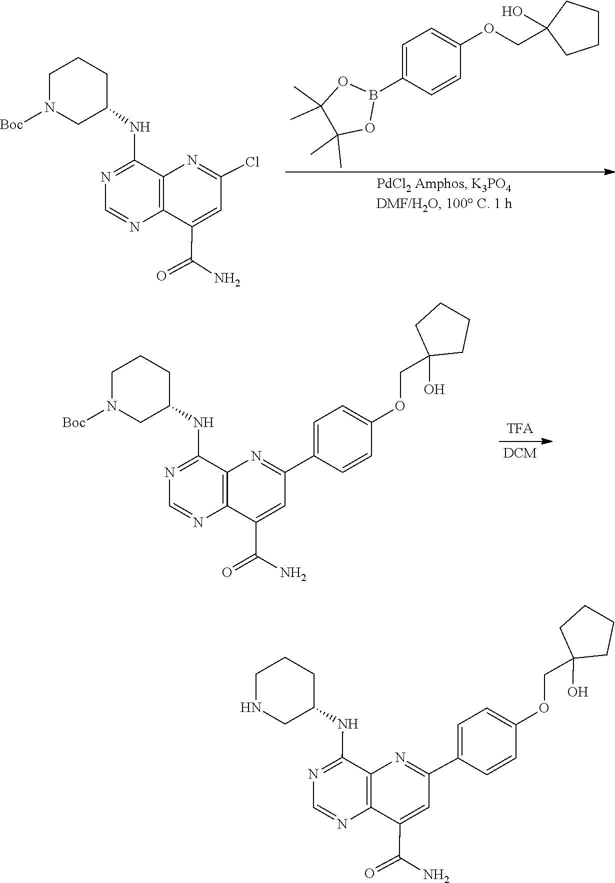 [SMILES: CC(C)(C)OC(=O)N1CCC[C@H](Nc2ncnc3c(C(N)=O)cc(Cl)nc23)C1 ; CC1(C)OB(c2ccc(OCC3(O)CCCC3)cc2)OC1(C)C ; CC(C)(C)OC(=O)N1CCC[C@H](Nc2ncnc3c(C(N)=O)cc(-c4ccc(OCC5(O)CCCC5)cc4)nc23)C1 ; NC(=O)c1cc(-c2ccc(OCC3(O)CCCC3)cc2)nc2c(N[C@H]3CCCNC3)ncnc12]

tert-butyl (3S)-3-[(8-carbamoyl-6-{4-[(1-hydroxycyclopentyl)methoxy]phenyl}pyrido[3,2-d]pyrimidin-4-yl)amino]piperidine-1-carboxylate: To a stirred mixture of 1-{[4-(4,4,5,5-tetramethyl-1,3,2-dioxaborolan-2-yl)phenoxy]methyl}cyclopentan-1-ol (500 mg; 0.68 mmol.) and tert-butyl (3S)-3-({8-carbamoyl-6-chloropyrido[3,2-d]pyrimidin-4-yl}amino)piperidine-1-carboxylate (200 mg; 0.40 mmol.) in DMF (12 ml) were added Pd(AMPHOS)Cl₂ (30 mg; 0.04 mmol., K₃PO₄ (270 mg; 1.21 mmol.) and H₂O (3 ml) in portions at 25 degrees C. The resulting mixture was stirred for 2 h at 100 degrees C. under nitrogen atmosphere. The resulting mixture was concentrated under vacuum. The residue was purified by silica gel column chromatography, eluted with PE:EA=1:1 to afford tert-butyl (3S)-3-[(8-carbamoyl-6-{4-[(1-hydroxycyclopentyl)methoxy]phenyl}pyrido[3,2-d]pyrimidin-4-yl)amino]piperidine-1-carboxylate as yellow solid (180 mg; 68.0%).

6-{4-[(1-hydroxycyclopentyl)methoxy]phenyl}-4-{[(3S)-piperidin-3-yl]amino}pyrido[3,2-d]pyrimidine-8-carboxamide: To a stirred mixture of tert-butyl (3S)-3-[(8-carbamoyl-6-{4-[(1-hydroxycyclopentyl)methoxy]phenyl}pyrido[3,2-d]pyrimidin-4-yl)amino]piperidine-1-carboxylate (150 mg; 0.23 mmol.) in DCM (5 ml) was added TFA (1 ml) dropwise at 25 degrees C. The resulting mixture was stirred for 2 h at 25 degrees C. under nitrogen atmosphere. The resulting mixture was concentrated under vacuum. The crude product was purified by Column: YMC-Actus Triart C18 ExRS, 30*150 mm, 5 μm; Mobile Phase A: Water (10 mmol/L NH₄HCO₃+0.05% NH₄OH), Mobile Phase B: ACN; Flow rate: 60 mL/min; Gradient: 25% B to 55% B in 8 min, 55% B; Wave Length: 254 nm; RT1 (min): 7; Number Of Runs: 2) to afford 6-{4-[(1-hydroxycyclopentyl)methoxy]phenyl}-4-{[(3S)-piperidin-3-yl]amino}pyrido[3,2-d]pyrimidine-8-carboxamide as yellow solid (51.6 mg; 48.8%).

HPLC: 99%, 3.54 min. M/S: m/z=463.25. ¹H NMR (400 MHz, DMSO-d6):10.07 (d, J=3.7 Hz, 1H), 8.80 (s, 1H), 8.54 (s, 1H), 8.32 (dd, J=8.4, 5.4 Hz, 3H), 8.22 (d, J=3.8 Hz, 1H), 7.15-7.08 (m, 2H), 4.63 (s, 1H), 4.28 (dq, J=9.4, 5.1, 4.6 Hz, 1H), 3.97 (s, 2H), 3.05 (dd, J=11.8, 3.8 Hz, 1H), 2.82 (dt, J=12.8, 4.0 Hz, 1H), 2.73 (dd, J=11.7, 8.6 Hz, 1H), 2.61-2.51 (m, 2H), 2.01-1.24 (m, 12H).

### Example 42: 4-(((3R,4R)-4-fluoropiperidin-3-yl)amino)-6-(4-(2-hydroxy-2-methylpropoxy)phenyl)pyrido[3,2-d]pyrimidine-8-carboxamide

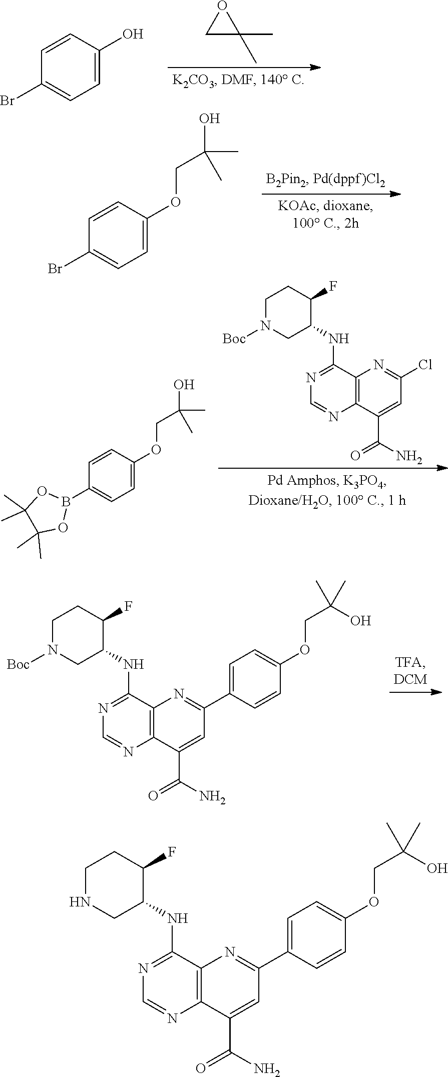

1-(4-bromophenoxy)-2-methylpropan-2-ol: To a solution of 4-bromophenol (10.00 g; 54.91 mmol.) and 2,2-dimethyloxirane (6.25 g; 82.34 mmol.) in DMF (100.00 ml) were added potassium Carbonate (23.95 g; 164.63 mmol) in portions at room temperature under nitrogen atmosphere. The resulting mixture was stirred for 3 h at 140 degrees C. under nitrogen atmosphere. The resulting mixture was added water (500 mL). The resulting mixture was extracted with EtOAc (3×300 mL). The combined organic layers were washed with brine (1×500 mL), dried over anhydrous Na₂SO₄. After filtration, the filtrate was concentrated under reduced pressure to afford 1-(4-bromophenoxy)-2-methylpropan-2-ol as a yellow oil (13.40 g; Crude Product).

2-methyl-1-[4-(4,4,5,5-tetramethyl-1,3,2-dioxaborolan-2-yl)phenoxy]propan-2-ol: To a stirred mixture of 1-(4-bromophenoxy)-2-methylpropan-2-ol (13.00 g; 51.31 mmol.) and BPD (27.43 g; 102.62 mmol.) in Dioxane-1,4 (130.00 ml) was added KOAc (15.90 g; 153.92 mmol.) and Pd(dppf)Cl₂ (3.95 g; 5.13 mmol.) in portions at room temperature. The resulting mixture was stirred for 1 h at 100 degrees C. under nitrogen atmosphere. The resulting mixture was concentrated under reduced pressure. The residue was purified by silica gel column chromatography, eluted with 8% EA in PE to afford 2-methyl-1-[4-(4,4,5,5-tetramethyl-1,3,2-dioxaborolan-2-yl)phenoxy]propan-2-ol as a yellow solid (9.32 g; 59.0%).

tert-butyl (3R,4R)-3-({8-carbamoyl-6-[4-(2-hydroxy-2-methylpropoxy)phenyl]pyrido[3,2-d]pyrimidin-4-yl}amino)-4-fluoropiperidine-1-carboxylate: To a stirred mixture of tert-butyl (3R,4R)-3-({8-carbamoyl-6-chloropyrido[3,2-d]pyrimidin-4-yl}amino)-4-fluoropiperidine-1-carboxylate (160 mg; 0.25 mmol.) and 2-methyl-1-[4-(4,4,5,5-tetramethyl-1,3,2-dioxaborolan-2-yl)phenoxy]propan-2-ol (221 mg; 0.72 mmol.) in 1,4-dioxane (10 ml) and H₂O (1 ml) was added K₃PO₄ (160 mg; 0.72 mmol.) and Pd(AMPHOS)Cl₂ (27 mg; 0.04 mmol) in portions at room temperature. The resulting mixture was stirred for 2 h at 100 degrees C. under nitrogen atmosphere. The resulting mixture was concentrated under reduced pressure. The residue was purified by silica gel column chromatography, eluted with 50% EA in PE to afford tert-butyl (3R,4R)-3-({8-carbamoyl-6-[4-(2-hydroxy-2-methylpropoxy)phenyl]pyrido[3,2-d]pyrimidin-4-yl}amino)-4-fluoropiperidine-1-carboxylate as a yellow solid (140 mg; 95.5%).

4-{[(3R,4R)-4-fluoropiperidin-3-yl]amino}-6-[4-(2-hydroxy-2-methylpropoxy)phenyl]pyrido[3,2-d]pyrimidine-8-carboxamide: To a stirred mixture of tert-butyl (3R,4R)-3-({8-carbamoyl-6-[4-(2-hydroxy-2-methylpropoxy)phenyl]pyrido[3,2-d]pyrimidin-4-yl}amino)-4-fluoropiperidine-1-carboxylate (120 mg; 0.20 mmol.) in DCM (3 ml) was added TFA (1 ml) dropwise at 0 degrees C. The resulting mixture was stirred for 1 h at room temperature under nitrogen atmosphere. The resulting mixture was concentrated under reduced pressure. The crude product was purified by Prep-HPLC with the following conditions (Column: YMC-Actus Triart C18 ExRS, 30*150 mm, 5 μm; Mobile Phase A: Water (10 mmol/L NH₄HCO₃+0.05% NH₄OH), Mobile Phase B: ACN; Flow rate: 60 mL/min; Gradient: 10% B to 40% B in 8 min, 40% B; Wave Length: 254 nm; RT1 (min): 7; Number Of Runs: 2) to afford 4-{[(3R,4R)-4-fluoropiperidin-3-yl]amino}-6-[4-(2-hydroxy-2-methylpropoxy)phenyl]pyrido[3,2-d]pyrimidine-8-carboxamide as a yellow solid (58 mg; 60.4%).

HPLC: 96%, 3.06 min. M/S: m/z=455.05. ¹H NMR (400 MHz, DMSO-d6) 10.03 (d, J=3.8 Hz, 1H), 8.83 (s, 1H), 8.66-8.48 (m, 2H), 8.38 (d, J=8.5 Hz, 2H), 8.22 (d, J=3.6 Hz, 1H), 7.13 (d, J=8.5 Hz, 2H), 5.15-4.90 (m, 1H), 4.69 (s, 1H), 4.59-4.37 (m, 1H), 3.84 (s, 2H), 3.33 (s, 1H), 3.21-3.09 (m, 1H), 3.01 (d, J=12.8 Hz, 1H), 2.67 (d, J=11.1 Hz, 1H), 2.55 (s, 1H), 2.15 (s, 1H), 1.63 (s, 1H), 1.25 (s, 6H).

### Example 43: (S)-6-(3,5-difluoro-4-((4-hydroxytetrahydro-2H-pyran-4-yl)methoxy)phenyl)-4-(piperidin-3-ylamino)pyrido[3,2-d]pyrimidine-8-carboxamide

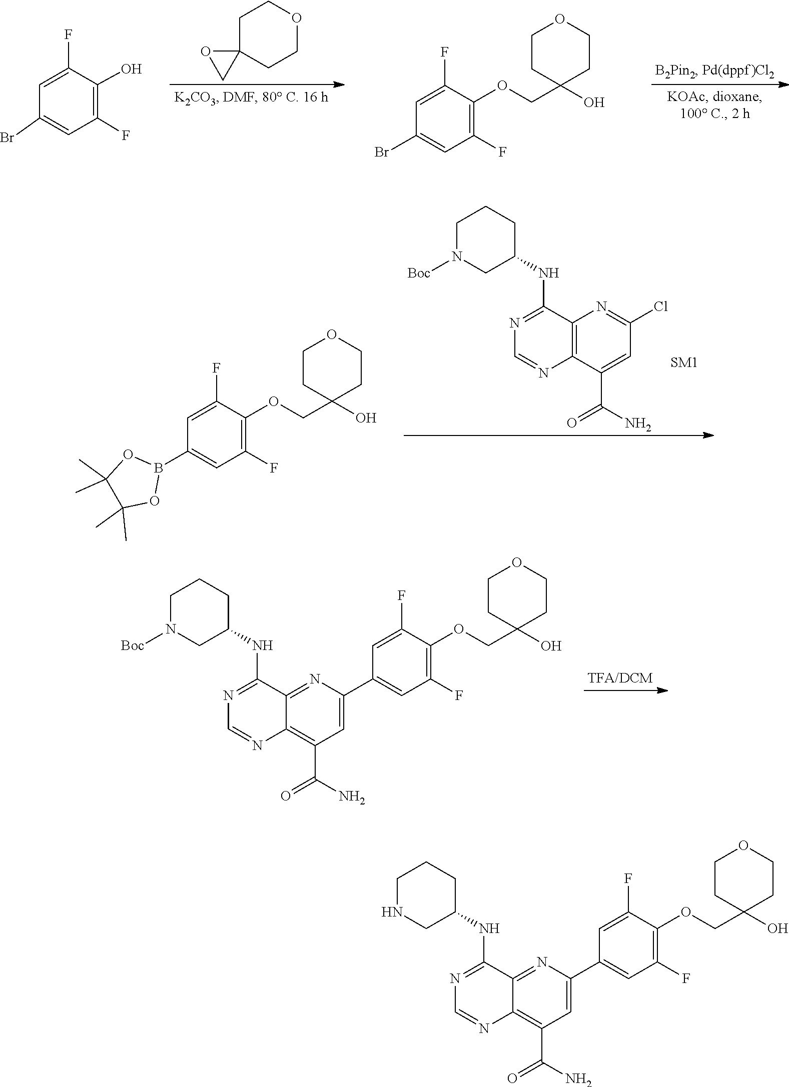

4-[(4-bromo-2,6-difluorophenoxy)methyl]oxan-4-ol: To a stirred mixture of 4-bromo-2,6-difluorophenol (1 g; 4.55 mmol) and 1,6-dioxaspiro[2.5]octane (1.64 g; 13.64 mmol) in DMF (15.00 ml) was added K₂CO₃ (1.98 g; 13.64 mmol) at room temperature. After stirring for 24 h at 80 degrees C. The resulting mixture was extracted with EtOAc and quenched with water. The combined organic layers were washed with brine, dried over anhydrous Na₂SO₄. After filtration, the filtrate was concentrated under reduced pressure. The crude product was purified by silica gel column chromatography, eluted with PE/EtOAc (7:3) to afford 4-[(4-bromo-2,6-difluorophenoxy)methyl]oxan-4-ol as a light yellow oil (1.45 g; 33.94%).

4-{[2,6-difluoro-4-(4,4,5,5-tetramethyl-1,3,2-dioxaborolan-2-yl)phenoxy]methyl}oxan-4-ol: To a stirred mixture of 4-[(4-bromo-2,6-difluorophenoxy)methyl]oxan-4-ol (1.4 g; 1.51 mmol) and BPD (1.21 g; 4.53 mmol) in Dioxane-1,4 (20 ml) were added KOAc (0.49 g; 4.53 mmol.) and Pd(dppf)Cl₂ (0.12 g; 0.15 mmol) at room temperature. After stirring for 3 h at 100 degrees C. under argon atmosphere. The resulting mixture was concentrated under reduced pressure and purified by silica gel column chromatography, eluted with PE/EtOAc (3:2) to afford 4-{[2,6-difluoro-4-(4,4,5,5-tetramethyl-1,3,2-dioxaborolan-2-yl)phenoxy]methyl}oxan-4-ol as a yellow solid (750 mg; 99.47%).

tert-butyl (3S)-3-[(8-carbamoyl-6-{3,5-difluoro-4-[(4-hydroxyoxan-4-yl)methoxy]phenyl}pyrido[3,2-d]pyrimidin-4-yl)amino]piperidine-1-carboxylate: To a stirred mixture of tert-butyl (3S)-3-({8-carbamoyl-6-chloropyrido[3,2-d]pyrimidin-4-yl}amino)piperidine-1-carboxylate (160 mg; 0.39 mmol.) and 4-{[2,6-difluoro-4-(4,4,5,5-tetramethyl-1,3,2-dioxaborolan-2-yl)phenoxy]methyl}oxan-4-ol (388 mg; 0.78 mmol) in Dioxane-1,4 (10 ml) and Water (2 ml) were added K₃PO₄ (260.69 mg; 1.17 mmol) and Bis(di-tert-butyl(4-dimethylaminophenyl)phosphine)dichloropalladium(II) (29 mg; 0.04 mmol) at room temperature. After stirring for 2 h at 100 degrees C. under argon atmosphere. The resulting mixture was concentrated under reduced pressure and purified by silica gel column chromatography, eluted with CH₂Cl₂/MeOH (4:1) to afford tert-butyl (3S)-3-[(8-carbamoyl-6-{3,5-difluoro-4-[(4-hydroxyoxan-4-yl)methoxy]phenyl}pyrido[3,2-d]pyrimidin-4-yl)amino]piperidine-1-carboxylate as a dark green solid (230 mg; 78.13%).

6-{3,5-difluoro-4-[(4-hydroxyoxan-4-yl)methoxy]phenyl}-4-{[(3S)-piperidin-3-yl]amino}pyrido[3,2-d]pyrimidine-8-carboxamide: To a solution of tert-butyl (3S)-3-[(8-carbamoyl-6-{3,5-difluoro-4-[(4-hydroxyoxan-4-yl)methoxy]phenyl}pyrido[3,2-d]pyrimidin-4-yl)amino]piperidine-1-carboxylate (220 mg; 0.29 mmol.) in DCM (5 ml) were added TFA (1 ml) at room temperature. After stirring for 1 h at room temperature. The resulting mixture was concentrated under reduced pressure and purified by silica gel column chromatography, eluted with CH₂Cl₂/MeOH (1:1). The crude product was purified by Prep-HPLC(Column: Xselect CSH OBD Column 30*150 mm 5 um; Mobile Phase A: Water (10 mmol/L NH₄HCO₃+0.05% NH₄OH), Mobile Phase B: ACN; Flow rate: 60 mL/min; Gradient: 16% B to 46% B in 10 min, 46% B; Wave Length: 254 nm; RT1 (min): 8.25; Number Of Runs: 2) to afford 6-{3,5-difluoro-4-[(4-hydroxyoxan-4-yl)methoxy]phenyl}-4-{[(3S)-piperidin-3-yl]amino}pyrido[3,2-d]pyrimidine-8-carboxamide as a yellow solid (67.5 mg; 45.06%).

HPLC: 99%, 3.03 min. M/S: m/z=515.20. ¹H NMR (300 MHz, DMSO-d6): 9.91 (s, 1H), 8.81 (s, 1H), 8.55 (s, 1H), 8.47 (d, J=8.6 Hz, 1H), 8.29-8.17 (m, 3H), 4.72 (s, 1H), 4.31 (s, 1H), 4.01 (s, 2H), 3.70-3.61 (m, 3H), 3.05 (d, J=12.1 Hz, 1H), 2.85 (d, J=12.4 Hz, 2H), 2.52 (s, 2H), 1.91 (s, 1H), 1.85-1.65 (m, 4H), 1.48 (d, J=12.8 Hz, 3H).

### Example 44: (S)-6-(4-((4-fluorotetrahydro-2H-pyran-4-yl)methyl)phenyl)-4-(piperidin-3-ylamino)pyrido[3,2-d]pyrimidine-8-carboxamide

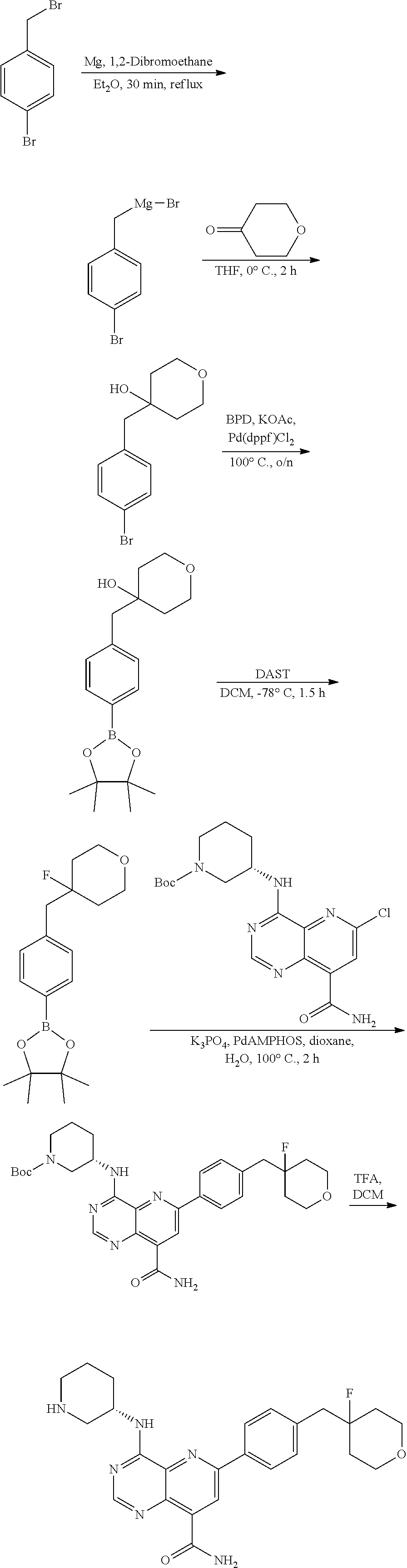

bromo[(4-bromophenyl)methyl]magnesium: To a flame-dried 250 mL three-necked round-bottom flask equipped with a dropping funnel and reflux condenser was charged activated Magnesium (0.7 g; 27.37 mmol) and 5 mL anhydrous Et₂O under a nitrogen atmosphere. The dropping funnel was charged with 1-bromo-4-(bromomethyl)benzene (6 g; 22.81 mmol) in anhydrous Et₂O (65 ml). A few drops of 1,2-dibromoethane (0.9 g; 4.56 mmol) was added to the reaction flask to initiate the reaction, and the bromide was added dropwise to maintain a steady reflux of solution. The addition was complete over 30 min. The solution was then cooled down and concentrated under reduced pressure to afford bromo[(4-bromophenyl)methyl]magnesium as a gray solution (6.5 g; 83.1%)

4-[(4-bromophenyl)methyl]oxan-4-ol: To a stirred solution of oxan-4-one (1.3 g; 12.34 mmol) in THF (20 ml) was added a solution of bromo[(4-bromophenyl)methyl]magnesium (6.50 g; 18.96 mmol) in Et₂O drop wised at 0 degree C. under N₂ atmosphere. The resulting mixture was stirred for 2 h at 0-degree C. under N₂ atmosphere. The resulting mixture was diluted with water (150 mL), extracted with EtOAc (3×100 mL). The combined organic layers were washed with brine (150 mL), dried over anhydrous Na₂SO₄. The resulting mixture was concentrated under reduced pressure. The residue was purified by silica gel column, eluted with 15% EtOAc in PE to afford crude product. The crude product was purified by C18 flash column eluted with 80% ACN in Water to afford 4-[(4-bromophenyl)methyl]oxan-4-ol as a white solid (800 mg; 15.56%)

4-{[4-(4,4,5,5-tetramethyl-1,3,2-dioxaborolan-2-yl)phenyl]methyl}oxan-4-ol: To a stirred solution of 4-[(4-bromophenyl)methyl]oxan-4-ol (780.00 mg; 2.01 mmol) and BPD (1030.06 mg; 4.02 mmol) in Dioxane-1,4 (6.00 ml) was added Pd(dppf)Cl₂ (154.65 mg; 0.20 mmol) and KOAc (622.28 mg; 6.02 mmol) in portions at room temperature under N₂ atmosphere. The resulting mixture was stirred for overnight at 100 degree C. under N₂ atmosphere. The resulting mixture was diluted with water (15 mL), extracted with EtOAc (3×10 mL). The combined organic layers were washed with brine (15 mL), dried over anhydrous Na₂SO₄. The resulting mixture was concentrated under reduced pressure. The residue was purified by silica gel column, eluted with 35% EtOAc in PE to afford 4-{[4-(4,4,5,5-tetramethyl-1,3,2-dioxaborolan-2-yl)phenyl]methyl}oxan-4-ol as a colorless oil (700 mg; 97.97%)

{4-[(4-fluorooxan-4-yl)methyl]phenyl}boronic acid: To a stirred solution of 4-{[4-(4,4,5,5-tetramethyl-1,3,2-dioxaborolan-2-yl)phenyl]methyl}oxan-4-ol (650 mg; 1.83 mmol) in DCM (5 mL) was added DAST (1.24 g; 7.31 mmol) in DCM (30 ml) dropwise at −78 degree C. under N₂ atmosphere. The resulting mixture was stirred for 1.5 h at −78 degree C. under N₂ atmosphere. The reaction was quenched with water and the mixture was extracted with EtOAc (3×15 mL). The combined organic layers were washed with brine, dried over anhydrous Na₂SO₄ and concentrated under reduced pressure to afford {4-[(4-fluorooxan-4-yl)methyl]phenyl}boronic acid as a yellow oil (650 mg; 71.01%)

tert-butyl (3S)-3-[(8-carbamoyl-6-{4-[(4-fluorooxan-4-yl)methyl]phenyl}pyrido[3,2-d]pyrimidin-4-yl)amino]piperidine-1-carboxylate: To a stirred solution of tert-butyl (3S)-3-({8-carbamoyl-6-chloropyrido[3,2-d]pyrimidin-4-yl}amino)piperidine-1-carboxylate (200 mg; 0.44 mmol), {4-[(4-fluorooxan-4-yl)methyl]phenyl}boronic acid (554.32 mg; 1.11 mmol) and K₃PO₄ (30 mg; 0.13 mmol) in Dioxane-1,4 (6 ml) and Water (1.5 ml) was added Bis(di-tert-butyl(4-dimethylaminophenyl)phosphine)dichloropalladium(II) (4 mg; 0.01 mmol) in portions at room temperature. The resulting mixture was stirred for 2 h at 100 degree C. under N₂ atmosphere. The resulting mixture was diluted with water (15 mL), extracted with EtOAc (3×10 mL). The combined organic layers were washed with brine (15 mL), dried over anhydrous Na₂SO₄. The resulting mixture was concentrated under reduced pressure.

The residue was purified by silica gel column, eluted with 75% EtOAc in PE to afford tert-butyl (3S)-3-[(8-carbamoyl-6-{4-[(4-fluorooxan-4-yl)methyl]phenyl}pyrido[3,2-d]pyrimidin-4-yl)amino]piperidine-1-carboxylate as a yellow solid (160 mg; 61.81%).

6-{4-[(4-fluorooxan-4-yl)methyl]phenyl}-4-{[(3S)-piperidin-3-yl]amino}pyrido[3,2-d]pyrimidine-8-carboxamide: To a stirred solution of tert-butyl (3S)-3-[(8-carbamoyl-6-{4-[(4-fluorooxan-4-yl)methyl]phenyl}pyrido[3,2-d]pyrimidin-4-yl)amino]piperidine-1-carboxylate (140 mg; 0.24 mmol) in DCM (3.00 ml) was added TFA (1.00 ml) dropwise. The resulting mixture was stirred for 1 h at 25 degree C. under N₂ atmosphere. The resulting mixture was concentrated under reduced pressure. The residue was purified by Prep-HPLC with the following condition:Column: XBridge Prep OBD C18 Column, 19*250 mm, 5 um; Mobile Phase A:Water (10 mmol/I NH₄HCO₃+0.05% NH₄OH), Mobile Phase B:ACN; Flow rate: 60 mL/min; Gradient: 20 B to 60 B in 8 min, 254 nm; RT1:6.5; Injection Number Of Runs: 2); to afford 6-{4-[(4-fluorooxan-4-yl)methyl]phenyl}-4-{[(3S)-piperidin-3-yl]amino}pyrido[3,2-d]pyrimidine-8-carboxamide as a white solid (60.50 mg; 54.01%)

HPLC: 99% purity, 4.564 min. MS:m/z=465.20[M+H]^{+ 1}H NMR (300 MHz, DMSO-d₆) 10.05 (d, J=3.6 Hz, 1H), 8.84 (s, 1H), 8.57 (s, 1H), 8.37 (d, J=8.6 Hz, 1H), 8.26 (dd, J=16.2, 5.8 Hz, 3H), 7.43 (d, J=7.9 Hz, 2H), 4.30 (s, 1H), 3.72 (t, J=7.6 Hz, 2H), 3.52 (t, J=11.4 Hz, 2H), 3.06 (d, J=23.0 Hz, 3H), 2.77 (dt, J=20.7, 10.4 Hz, 2H), 2.59 (d, J=10.8 Hz, 2H), 2.00-1.75 (m, 3H), 1.61 (dt, J=32.5, 16.5 Hz, 5H).

### Example 45: (S)-6-(6-methyl-7-oxo-5,6,7,8-tetrahydro-4H-pyrazolo[1,5-d][1,4]diazepin-2-yl)-4-(piperidin-3-ylamino)pyrido[3,2-d]pyrimidine-8-carboxamide

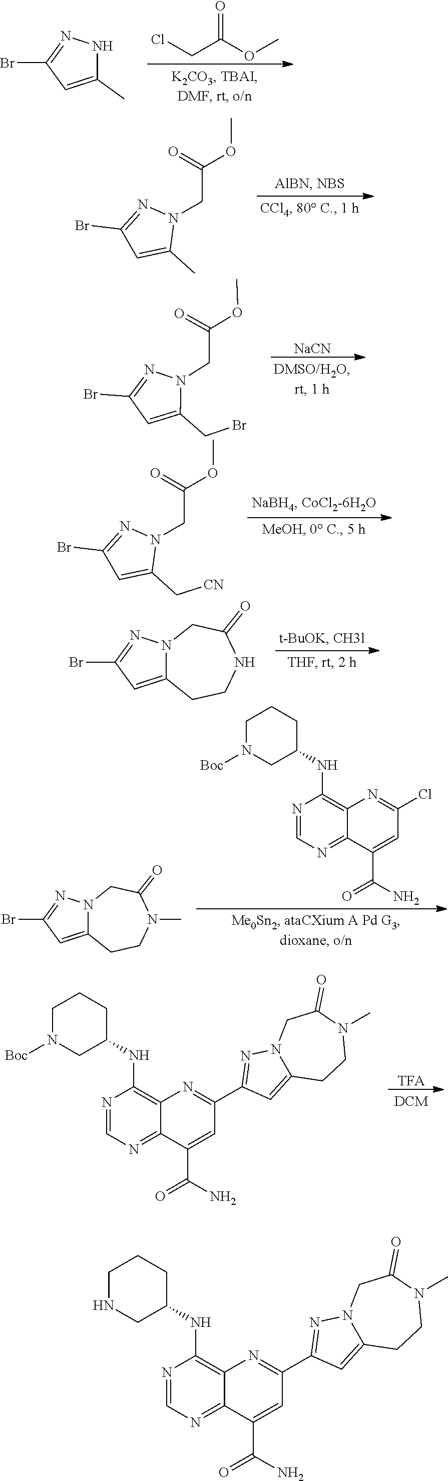

methyl 2-(3-bromo-5-methyl-1H-pyrazol-1-yl)acetate: To a stirred solution of 3-bromo-5-methyl-1H-pyrazole (50 g; 295.03 mmol) and K₂CO₃ (77.26 g; 531.07 mmol) in DMF (500 ml) was added methyl 2-chloroacetate (51 g; 446.45 mmol) dropwise at room temperature under N₂ atmosphere. The resulting mixture was stirred for overnight at 25 degree C. under N₂ atmosphere. The resulting mixture was poured into water (2 L), the mixture was extracted with EtOAc (600 ml×3). The combined organic layers were washed with brine dried over anhydrous Na₂SO₄ and concentrated under reduced pressure. The residue was purified by silica gel column eluted with PE:EtOAc=7:1 to afford methyl 2-(3-bromo-5-methyl-1H-pyrazol-1-yl)acetate as a white solid (65 g; 81.83%).

methyl 2-[3-bromo-5-(bromomethyl)-1H-pyrazol-1-yl]acetate: To a stirred solution of methyl 2-(3-bromo-5-methyl-1H-pyrazol-1-yl)acetate (25 g; 107.27 mmol) and NBS (20.1 g; 107.27 mmol) in Carbon tetrachloride (500 ml) was added 2-[(1Z)-2-(1-cyano-1-methylethyl)diazen-1-yl]-2-methylpropanenitrile (18.54 g; 107.27 mmol) in portions at room temperature under N₂ atmosphere. The resulting mixture was stirred for 3 h at 80 degree C. under N₂ atmosphere. The reaction mixture was diluted with water (1 L) and extracted with DCM (3×400 mL). The combined organic layers were washed with brine (500 mL), dried over anhydrous Na₂SO₄ and concentrated under reduced pressure. The residue was purified by silica gel column eluted with EtOAc:PE=8:1 to afford methyl 2-[3-bromo-5-(bromomethyl)-1H-pyrazol-1-yl]acetate as a yellow oil (22.00 g; 46.85%)

methyl 2-[3-bromo-5-(cyanomethyl)-1H-pyrazol-1-yl]acetate: To a stirred solution of methyl 2-[3-bromo-5-(bromomethyl)-1H-pyrazol-1-yl]acetate (22 g; 50.25 mmol) in DMSO (200 ml; 2815.82 mmol) were added Sodium cyanide (3.77 g; 75.38 mmol) and Water (4 ml; 219.76 mmol) in portions at room temperature under N₂ atmosphere. The resulting mixture was stirred for 1 h at 25 degree C. under N₂ atmosphere. The resulting mixture was quenched with FeSO₄ (aq.), extracted with EtOAc (3×100 mL). The combined organic layers were washed with brine (15 mL), dried over anhydrous Na₂SO₄. The resulting mixture was concentrated under reduced pressure. The residue was purified by silica gel column, eluted with PE:EA=7:1 and concentrated to afford methyl 2-[3-bromo-5-(cyanomethyl)-1H-pyrazol-1-yl]acetate as a light yellow solid (8.5 g; 57.01%)

2-bromo-4H,5H,6H,7H,8H-pyrazolo[1,5-d][1,4]diazepin-7-one: To a stirred solution of methyl 2-[3-bromo-5-(cyanomethyl)-1H-pyrazol-1-yl]acetate (1.5 g; 5.06 mmol) and Cobalt chloride hexahydrate (2.46 g; 10.11 mmol) in MeOH (50 ml) was added NaBH₄ (1.06 g; 25.28 mmol) in portions at 0 degree C. under N₂ atmosphere. The resulting mixture was stirred for 4 h at 25 degree C. under N₂ atmosphere. The reaction was quenched with water (50 mL) and the resulting mixture was concentrated under reduced pressure. The mixture was extracted with EtOAc (3×50 mL), the combined organic layers was dried over anhydrous Na₂SO₄ and concentrated under reduced pressure to afford 2-bromo-4H,5H,6H,7H,8H-pyrazolo[1,5-d][1,4]diazepin-7-one as a white solid (1.1 g; 64.27%)

2-bromo-6-methyl-4H,5H,6H,7H,8H-pyrazolo[1,5-d][1,4]diazepin-7-one: To a stirred solution of 2-bromo-4H,5H,6H,7H,8H-pyrazolo[1,5-d][1,4]diazepin-7-one (1.1 g; 3.07 mmol) and Mel (0.4 ml; 6.15 mmol) in THF (15 ml) was added Potassium tert-butoxide solution 1.0 M in THF (5.99 ml; 6.15 mmol) at room temperature under N₂ atmosphere. The resulting mixture was stirred for 3 h at 25 degree C. under N₂ atmosphere. The resulting mixture was diluted with water (50 mL). The solution was extracted with EtOAc (3×30 mL). The combined organic layers were washed with brine dried over anhydrous Na₂SO₄ and concentrated under reduced pressure. The residue was purified by C18 flash eluted with 67% ACN in water to afford 2-bromo-6-methyl-4H,5H,6H,7H,8H-pyrazolo[1,5-d][1,4]diazepin-7-one as a white solid (700.00 mg; 91.38%;)

tert-butyl (3S)-3-[(8-carbamoyl-6-{6-methyl-7-oxo-4H,5H,6H,7H,8H-pyrazolo[1,5-d][1,4]diazepin-2-yl}pyrido[3,2-d]pyrimidin-4-yl)amino]piperidine-1-carboxylate: To a stirred solution of 2-bromo-6-methyl-4H,5H,6H,7H,8H-pyrazolo[1,5-d][1,4]diazepin-7-one (140 mg; 0.56 mmol), tert-butyl (3S)-3-({8-carbamoyl-6-chloropyrido[3,2-d]pyrimidin-4-yl}amino)piperidine-1-carboxylate (259 mg; 0.60 mmol) and hexamethyldistannane (245 mg; 0.74 mmol) in 1,4-dioxane (5 ml) was added cataCXium A Pd G3 (42 mg; 0.05 mmol) in portions at room temperature under N₂ atmosphere. The resulting mixture was stirred 6 h at 100 degree C. under N₂ atmosphere. The resulting mixture was diluted with water (15 mL), extracted with EtOAc (3×10 mL). The combined organic layers were washed with brine (15 mL), dried over anhydrous Na₂SO₄. The resulting mixture was concentrated under reduced pressure. The residue was purified by silica gel column eluted with 75% EtOAc in PE to afford tert-butyl (3S)-3-[(8-carbamoyl-6-{6-methyl-7-oxo-4H,5H,6H,7H,8H-pyrazolo[1,5-d][1,4]diazepin-2-yl}pyrido[3,2-d]pyrimidin-4-yl)amino]piperidine-1-carboxylate as a yellow solid (180 mg; 57.76%)

6-{6-methyl-7-oxo-4H,5H,6H,7H,8H-pyrazolo[1,5-d][1,4]diazepin-2-yl}-4-{[(3S)-piperidin-3-yl]amino}pyrido[3,2-d]pyrimidine-8-carboxamide: To a stirred solution of tert-butyl (3S)-3-[(8-carbamoyl-6-{6-methyl-7-oxo-4H,5H,6H,7H,8H-pyrazolo[1,5-d][1,4]diazepin-2-yl}pyrido[3,2-d]pyrimidin-4-yl)amino]piperidine-1-carboxylate (160 mg; 0.29 mmol) in DCM (4.5 ml) was added TFA (1.5 ml) dropwise at 0 degree C. The resulting mixture was stirred for 1 at 25 degree C. under N₂ atmosphere. The resulting mixture was concentrated under reduced pressure. The residue was purified by Prep-HPLC with the following condition: Column: Xselect CSH OBD Column 30*150 mm Sum; Mobile Phase A:Water (10 mmol/l NH₄HCO₃+0.05% NH₄OH), Mobile Phase B:ACN; Flow rate: 60 mL/min; Gradient: 10 B to 40 B in 8 min, 254 nm; RT1:6.8; Injection Number Of Runs: 2); to afford 6-{6-methyl-7-oxo-4H,5H,6H,7H,8H-pyrazolo[1,5-d][1,4]diazepin-2-yl}-4-{[(3S)-piperidin-3-yl]amino}pyrido[3,2-d]pyrimidine-8-carboxamide as off-white solid (49.6 mg; 37.72%)

HPLC: 95% purity, 3.026 min. MS:m/z=436.10[M+H]^{+ 1}H NMR (400 MHz, DMSO-d6) 10.11 (d, J=3.8 Hz, 1H), 8.89 (s, 1H), 8.55 (s, 1H), 8.24 (dd, J=10.0, 6.2 Hz, 2H), 7.19 (s, 1H), 5.23 (s, 2H), 4.27 (m, J=8.4, 4.3, 3.8 Hz, 1H), 3.97-3.89 (m, 3H), 3.21 (t, J=5.9 Hz, 2H), 3.06 (dd, J=12.1, 3.8 Hz, 1H), 3.00 (s, 3H), 2.83 (m, J=13.6, 4.0 Hz, 1H), 2.70 (dd, J=11.7, 8.8 Hz, 1H), 2.57 (d, J=11.9 Hz, 1H), 1.97-1.85 (m, 1H), 1.79 (m, J=12.7, 11.4, 6.8 Hz, 1H), 1.72-1.65 (m, 1H), 1.49 (m, J=13.6, 5.7 Hz, 1H).

### Example 46: (S)-6-(2-cyano-4-(2-methoxyethoxy)phenyl)-4-(piperidin-3-ylamino)pyrido[3,2-d]pyrimidine-8-carboxamide

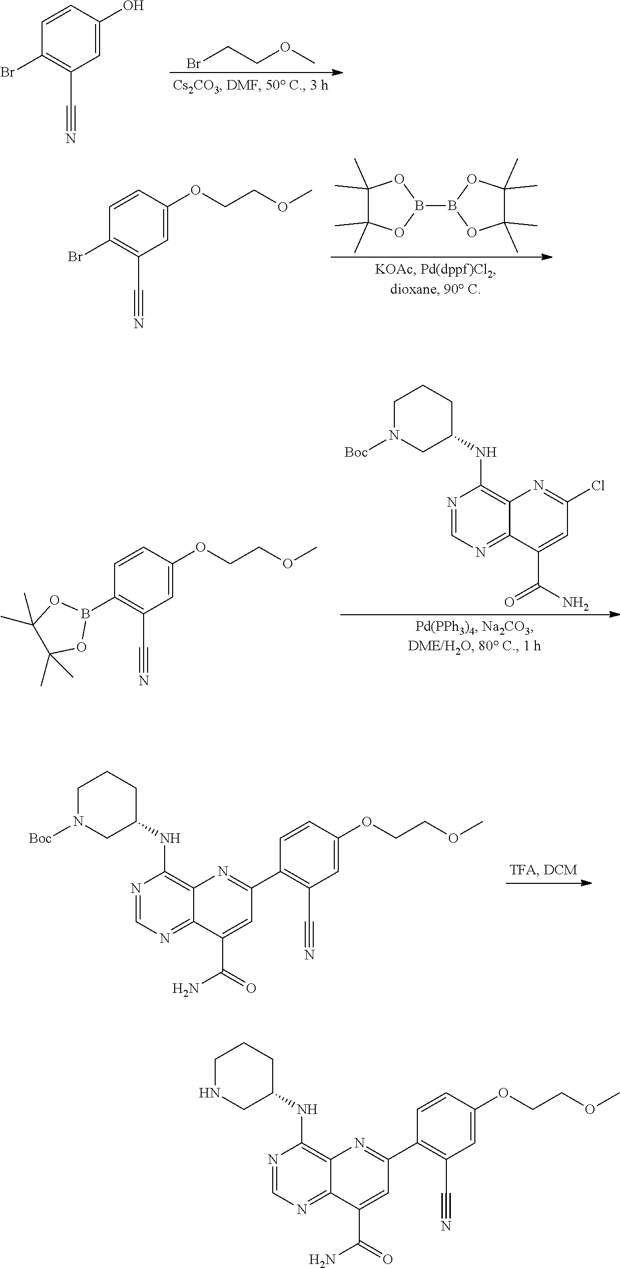

2-bromo-5-(2-methoxyethoxy)benzonitrile: To a stirred solution of 2-bromo-5-hydroxybenzonitrile (2 g; 9.60 mmol) and 1-bromo-2-methoxyethane (2.80 g; 19.14 mmol) in DMF (20 ml) was added K₂CO₃ (2.80 g; 19.25 mmol). The resulting mixture was stirred for overnight at 50 degrees C. The mixture was allowed to cool down to room temperature. The resulting mixture was diluted with water (30 mL) and extracted with Ethyl acetate (3×20 mL). The combined organic layers were washed with brine (20 mL), dried over anhydrous Na₂SO₄. After filtration, the filtrate was concentrated under reduced pressure. The residue was purified by silica gel column chromatography, eluted with petroleum ether/Ethyl acetate (5:1) to afford 2-bromo-5-(2-methoxyethoxy)benzonitrile as a light yellow solid (2 g; 81.4%).

5-(2-methoxyethoxy)-2-(4,4,5,5-tetramethyl-1,3,2-dioxaborolan-2-yl)benzonitrile: To a stirred solution of 2-bromo-5-(2-methoxyethoxy)benzonitrile (1 g; 3.90 mmo) and BPD (1.50 g; 5.61 mmol) in Dioxane-1,4 (10 ml) was added KOAc (1.1 g; 10.65 mmol) and Pd(dppf)Cl₂ (0.3 g; 0.39 mmol). The resulting mixture was stirred for 2 h at 100 degrees C. under nitrogen atmosphere. The mixture was allowed to cool down to room temperature. The resulting mixture was diluted with water (20 mL) and extracted with Ethyl acetate (3×20 mL). The combined organic layers were washed with brine (20 mL), dried over anhydrous Na₂SO₄. After filtration, the filtrate was concentrated under reduced pressure. The residue was purified by silica gel column chromatography, eluted with petroleum ether/Ethyl acetate (9:1) to afford 5-(2-methoxyethoxy)-2-(4,4,5,5-tetramethyl-1,3,2-dioxaborolan-2-yl)benzonitrile as a light yellow oil (800 mg; 61.2%).

tert-butyl (3S)-3-({8-carbamoyl-6-[2-cyano-4-(2-methoxyethoxy)phenyl]pyrido[3,2-d]pyrimidin-4-yl}amino)piperidine-1-carboxylate: To a stirred solution of 5-(2-methoxyethoxy)-2-(4,4,5,5-tetramethyl-1,3,2-dioxaborolan-2-yl)benzonitrile (299 mg; 0.89 mmol), Na₂CO₃ (156 mg; 1.40 mmol) and tert-butyl (3S)-3-({8-carbamoyl-6-chloropyrido[3,2-d]pyrimidin-4-yl}amino)piperidine-1-carboxylate (200 mg; 0.49 mmol) in DME (3 ml) and H₂O (1 ml) was added Pd(PPh₃)₄ (57 mg; 0.05 mmol). The resulting mixture was stirred for 2 h at 80 degrees C. under nitrogen atmosphere. The resulting mixture was diluted with water (20 mL) and extracted with Ethyl acetate (3×20 mL). The combined organic layers were washed with brine (20 mL), dried over anhydrous Na₂SO₄. After filtration, the filtrate was concentrated under reduced pressure. The residue was purified by Prep-TLC (petroleum ether/Ethyl acetate) (2:1) to afford tert-butyl (3S)-3-({8-carbamoyl-6-[2-cyano-4-(2-methoxyethoxy)phenyl]pyrido[3,2-d]pyrimidin-4-yl}amino)piperidine-1-carboxylate as a light yellow solid (120 mg; 42.5%).

6-[2-cyano-4-(2-methoxyethoxy)phenyl]-4-{[(3S)-piperidin-3-yl]amino}pyrido[3,2-d]pyrimidine-8-carboxamide: To a stirred solution of tert-butyl (3S)-3-({8-carbamoyl-6-[2-cyano-4-(2-methoxyethoxy)phenyl]pyrido[3,2-d]pyrimidin-4-yl}amino)piperidine-1-carboxylate (100 mg; 0.17 mmol) in DCM (1 ml) was added TFA (0.2 ml) dropwise at 0 degrees C. The resulting mixture was stirred for 2 h at room temperature. he resulting mixture was concentrated under vacuum. The crude product was purified by Prep-HPLC with the following conditions (Column: XBridge Prep OBD C18 Column, 30×150 mm 5 um; Mobile Phase A: Water (10 mmol/I NH₄HCO₃+0.05% NH₄OH), Mobile Phase B:ACN; Flow rate: 60 mL/min; Gradient: 20 B to 50 B in 8 min, 254 nm) to afford 6-[2-cyano-4-(2-methoxyethoxy)phenyl]-4-{[(3S)-piperidin-3-yl]amino}pyrido[3,2-d]pyrimidine-8-carboxamide as a yellow solid (55.50 mg; 70.4%)

HPLC: 98% purity, 4.44 min. MS:m/z=448.15 [M+H]⁺. ¹H NMR (300 MHz, DMSO-d6) 9.95 (s, 1H), 8.80 (s, 1H), 8.61 (s, 1H), 8.23 (d, J=9.1 Hz, 2H), 7.99 (d, J=8.3 Hz, 1H), 7.64 (d, J=2.7 Hz, 1H), 7.45 (dd, J=8.9, 2.7 Hz, 1H), 4.33-4.25 (m, 2H), 4.22 (s, 1H), 3.75-3.66 (m, 2H), 3.33 (s, 3H), 3.10 (d, J=11.3 Hz, 1H), 2.79 (d, J=12.2 Hz, 1H), 2.65-2.51 (m, 2H), 1.95 (s, 1H), 1.84-1.43 (m, 2H).

### Example 47: (S)-6-(4-(1,1-difluoro-2-hydroxy-2-methylpropoxy)phenyl)-4-(piperidin-3-ylamino)pyrido[3,2-d]pyrimidine-8-carboxamide

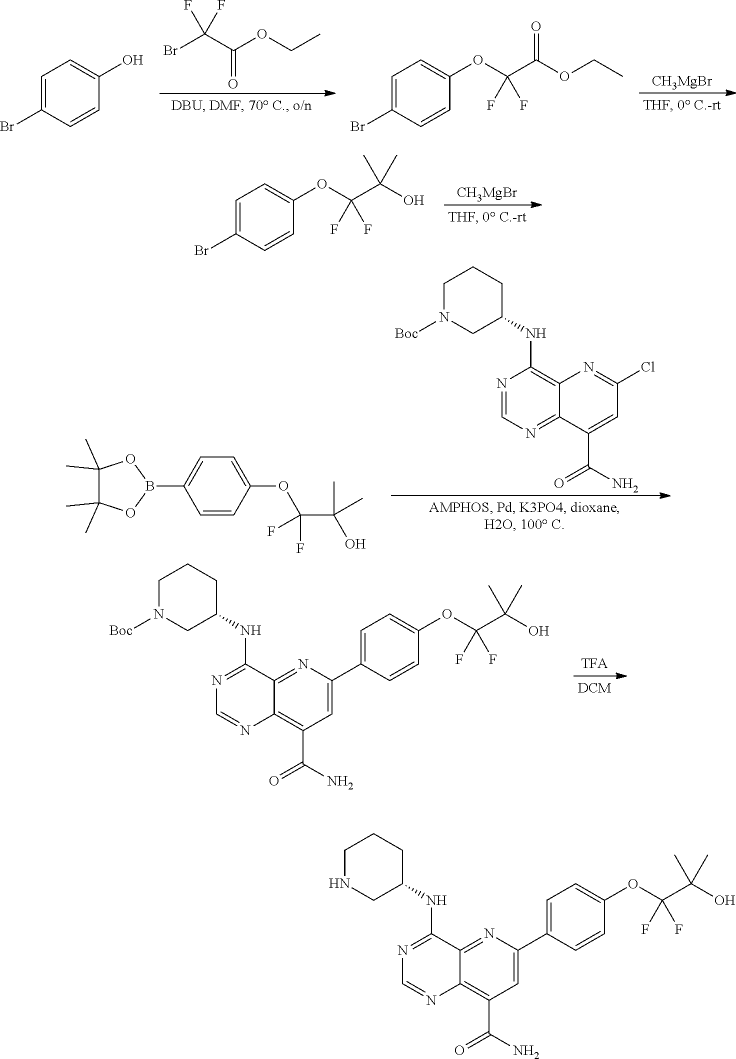

ethyl 2-(4-bromophenoxy)-2,2-difluoroacetate: To a solution of 4-bromophenol (3.00 g; 16.47 mmol) in DMF (10.00 ml) was added DBU (6.63 g; 41.37 mmol) in portions at room temperature. The mixture was heated to 70 degrees C. and ethyl 2-bromo-2,2-difluoroacetate (8.81 g; 41.23 mmol) was added in. The resulting mixture was stirred for overnight at 70 degrees C. under nitrogen. The resulting mixture was diluted 200 ml water and extracted with EA (3×150 mL). The combined organic layers were washed with brine, dried over anhydrous Na₂SO₄. After filtration, the filtrate was concentrated under reduced pressure. The residue was purified by silica gel column chromatography, eluted with 4% EA in PE to afford ethyl 2-(4-bromophenoxy)-2,2-difluoroacetate as a yellow liquid (2.47 g; 50.8%).

1-(4-bromophenoxy)-1,1-difluoro-2-methylpropan-2-ol: To a stirred mixture of ethyl 2-(4-bromophenoxy)-2,2-difluoroacetate (2.00 g; 6.78 mmol) in THF (10.00 ml) was added bromo(methyl)magnesium (5.67 ml; 17.01 mmol) dropwise at 0 degrees C. The resulting mixture was stirred for 3 h at room temperature under nitrogen atmosphere. The reaction was quenched with water at 0 degrees C. The mixture was extracted with EA (3×50 mL). The combined organic layers were washed with brine, dried over anhydrous Na₂SO₄. After filtration, the filtrate was concentrated under reduced pressure. The residue was purified by silica gel column chromatography, eluted with 8% EA in PE. The residue was purified by reverse flash chromatography with the following conditions: column, C18 silica gel; mobile phase, ACN in water, 45% to 50% gradient in 10 min; detector, UV 220 nm to afford 1-(4-bromophenoxy)-1,1-difluoro-2-methylpropan-2-ol as a brown liquid (1.37 g; 70.4%).

(3R)-4-methyl-3-[4-(4,4,5,5-tetramethyl-1,3,2-dioxaborolan-2-yl)phenyl]morpholine: To a stirred mixture of 1-(4-bromophenoxy)-1,1-difluoro-2-methylpropan-2-ol (1.33 g; 4.63 mmol) and BPD (2.46 g; 9.20 mmol) in Dioxane-1,4 (10.00 ml) was added KOAc (0.96 g; 9.29 mmol) and Pd(dppf)Cl₂ (0.38 g; 0.49 mmol) in portions at room temperature. The resulting mixture was stirred for 1 h at 100 degrees C. under nitrogen atmosphere. The resulting mixture was concentrated under reduced pressure. The residue was purified by silica gel column chromatography, eluted with 70% DCM in PE to afford (3R)-4-methyl-3-[4-(4,4,5,5-tetramethyl-1,3,2-dioxaborolan-2-yl)phenyl]morpholine as a white solid (1.53 g; 86.5%;)

tert-butyl(3S)-3-({8-carbamoyl-6-[4-(1,1-difluoro-2-hydroxy-2-methylpropoxy)phenyl]pyrido[3,2-d]pyrimidin-4-yl}amino)piperidine-1-carboxylate: To a stirred mixture of tert-butyl (S)-3-((8-carbamoyl-6-chloropyrido[3,2-d]pyrimidin-4-yl)amino)piperidine-1-carboxylate (230 mg; 0.53 mmol) and (-4-methyl-3-[4-(4,4,5,5-tetramethyl-1,3,2-dioxaborolan-2-yl)phenyl]morpholine (279.00 mg; 0.73 mmol) in 1,4-dioxane (3 ml) and H₂O (0.3 ml) was added K₃PO₄ (361 mg; 1.62 mmol) and Pd(AMPHOS)Cl₂ (41.00 mg; 0.06 mmol) in portions at room temperature. The resulting mixture was stirred for 1 h at 100 degrees C. under nitrogen atmosphere. The resulting mixture was concentrated under reduced pressure. The residue was purified by silica gel column chromatography, eluted with 40% EA in PE to afford tert-butyl(3S)-3-({8-carbamoyl-6-[4-(1,1-difluoro-2-hydroxy-2-methylpropoxy)phenyl]pyrido[3,2-d]pyrimidin-4-yl}amino)piperidine-1-carboxylate as a yellow solid (200 mg; 46.6%)

6-[4-(1,1-difluoro-2-hydroxy-2-methylpropoxy)phenyl]-4-{[(3S)-piperidin-3-yl]amino}pyrido[3,2-d]pyrimidine-8-carboxamide: To a stirred mixture of tert-butyl (3S)-3-({8-carbamoyl-6-[4-(1,1-difluoro-2-hydroxy-2-methylpropoxy)phenyl]pyrido[3,2-d]pyrimidin-4-yl}amino)piperidine-1-carboxylate (190 mg; 0.23 mmol) in DCM (3 ml) was added TFA (1 ml) dropwise at 0 degrees C. The resulting mixture was stirred for 1 h at room temperature under nitrogen atmosphere. The resulting mixture was concentrated under reduced pressure. The crude product was purified by Prep-HPLC with the following conditions (Column: XBridge Prep OBD C18 Column, 30×150 mm 5 um; Mobile Phase A:Water (10 mmol/I NH₄HCO₃+0.05% NH₄OH), Mobile Phase B:ACN; Flow rate: 60 mL/min; Gradient: 28 B to 58 B in 10 min; 254 nm; RT1:7.3) to afford 6-[4-(1,1-difluoro-2-hydroxy-2-methylpropoxy)phenyl]-4-{[(3S)-piperidin-3-yl]amino}pyrido[3,2-d]pyrimidine-8-carboxamide as a yellow solid (62.50 mg; 53.2%)

HPLC: 94% purity, 3.221 min. MS:m/z=473.10[M+H]⁺. ¹H NMR (300 MHz, DMSO-d6) 10.04 (d, J=3.8 Hz, 1H), 8.83 (s, 1H), 8.56 (s, 1H), 8.39 (dd, J=12.8, 8.8 Hz, 3H), 8.22 (d, J=3.7 Hz, 1H), 7.37 (d, J=8.4 Hz, 2H), 5.57 (s, 1H), 4.27 (s, 1H), 3.05 (dd, J=12.0, 3.8 Hz, 1H), 2.87-2.65 (m, 2H), 2.61-2.51 (m, 2H), 1.94-1.86 (m, 1H), 1.80 (d, J=10.1 Hz, 1H), 1.68 (d, J=11.8 Hz, 1H), 1.50 (s, 1H), 1.37 (s, 6H).

### Example 48 & 49: 4-(((3R,4R)-4-fluoropiperidin-3-yl)amino)-6-(4-(2-methoxyethoxy)phenyl)pyrido[3,2-d]pyrimidine-8-carboxamide and 4-(((3S,4S)-4-fluoropiperidin-3-yl)amino)-6-(4-(2-methoxyethoxy)phenyl)pyrido[3,2-d]pyrimidine-8-carboxamide

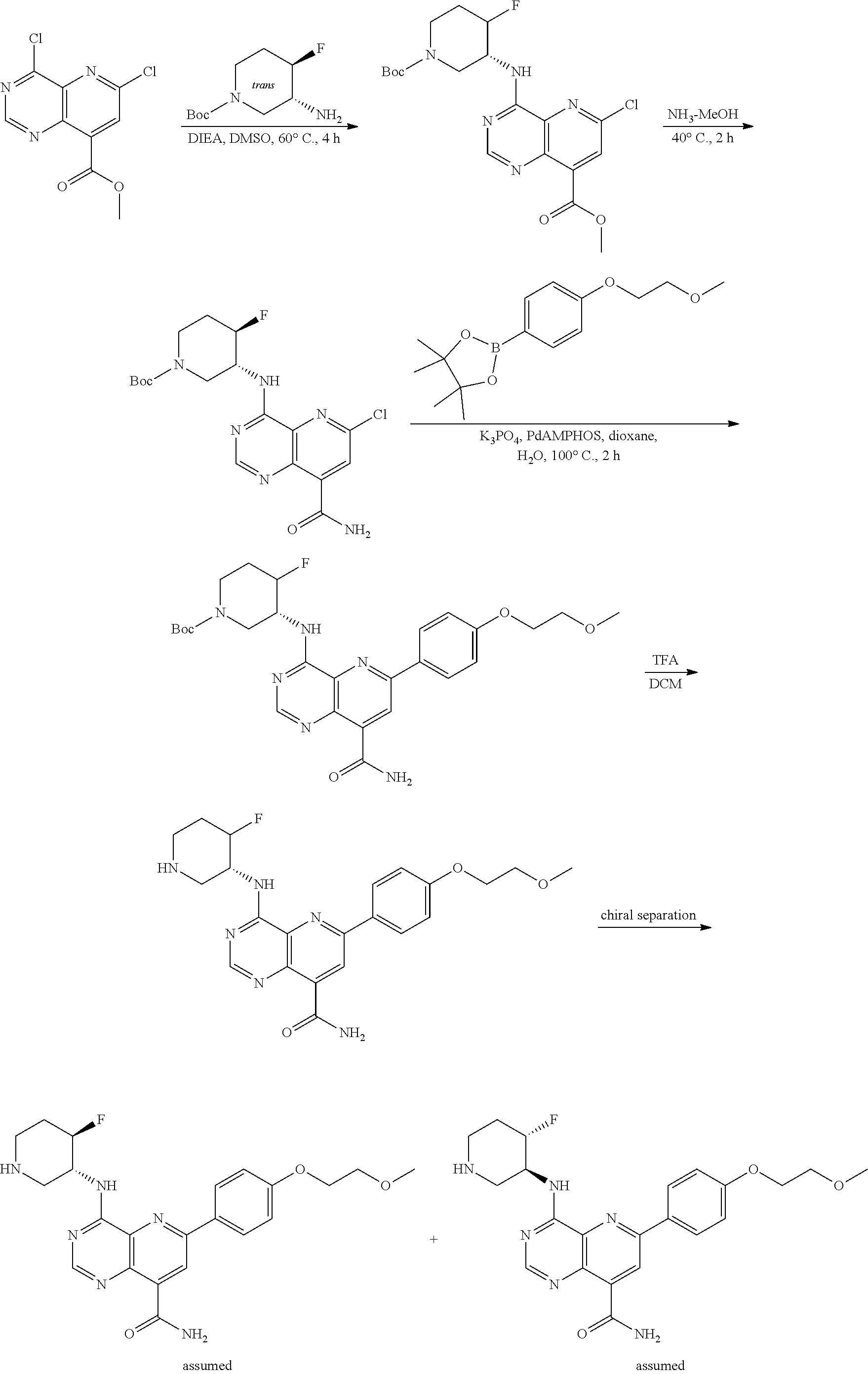

tert-butyl (3R,4R)-3-{[6-chloro-8-(methoxycarbonyl)pyrido[3,2-d]pyrimidin-4-yl]amino}-4-fluoropiperidine-1-carboxylate: To a stirred mixture of methyl 4,6-dichloropyrido[3,2-d]pyrimidine-8-carboxylate (3 g; 9.99 mmol) and tert-butyl-3-amino-4-fluoropiperidine-1-carboxylate(trans mixture) (2.75 g; 11.98 mmol) in DMSO (20 ml) was added DIEA (4.07 g; 29.96 mmol) at room temperature under argon atmosphere. The resulting mixture was stirred for overnight at 60 degrees C. under argon atmosphere. The residue was purified by silica gel column chromatography, eluted with PE/EtOAc (3:2) to afford tert-butyl (3R,4R)-3-{[6-chloro-8-(methoxycarbonyl)pyrido[3,2-d]pyrimidin-4-yl]amino}-4-fluoropiperidine-1-carboxylate (3.40 g; 76.2%) as a yellow solid.

tert-butyl (3R,4R)-3-({8-carbamoyl-6-chloropyrido[3,2-d]pyrimidin-4-yl}amino)-4-fluoropiperidine-1-carboxylate: A mixture of tert-butyl (3R,4R)-3-{[6-chloro-8-(methoxycarbonyl)pyrido[3,2-d]pyrimidin-4-yl]amino}-4-fluoropiperidine-1-carboxylate (3.40 g; 7.61 mmol) and NH₃ (g) in MeOH (20.00 ml) was stirred for overnight at 60 degrees C. under argon atmosphere. The resulting mixture was concentrated under vacuum. This resulted in tert-butyl (3R,4R)-3-({8-carbamoyl-6-chloropyrido[3,2-d]pyrimidin-4-yl}amino)-4-fluoropiperidine-1-carboxylate (3.10 g; 92.5%) as a yellow solid.

tert-butyl (3R,4R)-3-({8-carbamoyl-6-[4-(2-methoxyethoxy)phenyl]pyrido[3,2-d]pyrimidin-4-yl}amino)-4-fluoropiperidine-1-carboxylate: To a stirred mixture of tert-butyl (3R,4R)-3-({8-carbamoyl-6-chloropyrido[3,2-d]pyrimidin-4-yl}amino)-4-fluoropiperidine-1-carboxylate (250 mg; 0.57 mmol) and [4-(2-methoxyethoxy)phenyl]boronic acid (175.74 mg; 0.85 mmol) in Dioxane-1,4 (10 ml) and Water (2 ml) were added tert-butyl (3R,4R)-3-({8-carbamoyl-6-[4-(2-methoxyethoxy)phenyl]pyrido[3,2-d]pyrimidin-4-yl}amino)-4-fluoropiperidine-1-carboxylate (310 mg; 0.40 mmol; 70.58%; yellow solid; Purified Product) and Bis(di-tert-butyl(4-dimethylaminophenyl)phosphine)dichloropalladium(II) (42.33 mg; 0.06 mmol) at room temperature. After stirring for 2 h at 100 degrees C. under argon atmosphere. The residue was purified by silica gel column chromatography, eluted with CH₂Cl₂/MeCOH (19:1) to afford tert-butyl (3R,4R)-3-({8-carbamoyl-6-[4-(2-methoxyethoxy)phenyl]pyrido[3,2-d]pyrimidin-4-yl}amino)-4-fluoropiperidine-1-carboxylate as a yellow solid (310 mg; 70.58%).

4-{[(3R,4R)-4-fluoropiperidin-3-yl]amino}-6-[4-(2-methoxyethoxy)phenyl]pyrido[3,2-d]pyrimidine-8-carboxamide: To a stirred mixture of tert-butyl (3R,4R)-3-({8-carbamoyl-6-[4-(2-methoxyethoxy)phenyl]pyrido[3,2-d]pyrimidin-4-yl}amino)-4-fluoropiperidine-1-carboxylate (300 mg; 0.34 mmol) and TFA (5 ml) in DCM (15 ml) at room temperature. After stirring for 1 h at room temperature. The crude product was purified by Prep-HPLC(Column: CHIRALPAK ID, 2*25 cm, 5 um; Mobile Phase A: MTBE (0.5% 2M NH₃-MeOH)-HPLC, Mobile Phase B:IPA-HPLC; Flow rate: 20 mL/min; Gradient: 30 B to 30 B in 18 min; 220/254 nm; RT1:9.301; RT2:14.362; Injection Volume: 2 ml; Number Of Runs: 5;) to afford 4-{[(3R,4R)-4-fluoropiperidin-3-yl]amino}-6-[4-(2-methoxyethoxy)phenyl]pyrido[3,2-d]pyrimidine-8-carboxamide as a yellow green solid (120 mg 78.83%).

4-{[(3R,4R)-4-fluoropiperidin-3-yl]amino}-6-[4-(2-methoxyethoxy)phenyl]pyrido[3,2-d]pyrimidine-8-carboxamide&4-{[(3S,4S)-4-fluoropiperidin-3-yl]amino}-6-[4-(2-methoxyethoxy)phenyl]pyrido[3,2-d]pyrimidine-8-carboxamide: The product 4-{[(3R,4R)-4-fluoropiperidin-3-yl]amino}-6-[4-(2-methoxyethoxy)phenyl]pyrido[3,2-d]pyrimidine-8-carboxamide (120.00 mg; 0.27 mmol) was purified by Prep-Chiral-HPLC. The product 4-{[(3R,4R)-4-fluoropiperidin-3-yl]amino}-6-[4-(2-methoxyethoxy)phenyl]pyrido[3,2-d]pyrimidine-8-carboxamide (120 mg; 0.27 mmol) was purified by Prep-Chiral-HPLC(Column: CHIRALPAK ID, 2*25 cm, 5 um; Mobile Phase A: MTBE (0.5% 2M NH₃-MeOH)-HPLC, Mobile Phase B:IPA-HPLC; Flow rate: 18 mL/min; Gradient: 30 B to 30 B in 21 min; 220/254 nm; RT1:10.597; RT2:19.055; Injection Volume: 2.4 ml; Number Of Runs: 3;) to afford 4-{[(3R,4R)-4-fluoropiperidin-3-yl]amino}-6-[4-(2-methoxyethoxy)phenyl]pyrido[3,2-d]pyrimidine-8-carboxamide as a yellow solid (45.20 mg; 37.44%) and 4-{[(3S,4S)-4-fluoropiperidin-3-yl]amino}-6-[4-(2-methoxyethoxy)phenyl]pyrido[3,2-d]pyrimidine-8-carboxamide as a yellow solid (37.70 mg; 31.20%).

Ena1: HPLC: 98% purity, 3.068 min. MS: m/z=441.10[M+H]⁺. ¹H NMR (300 MHz, DMSO-d6): 10.01 (d, J=3.5 Hz, 1H), 8.81 (s, 1H), 8.63-8.53 (m, 2H), 8.43-8.33 (m, 2H), 8.22 (d, J=3.6 Hz, 1H), 7.12 (d, J=8.8 Hz, 2H), 5.10 (s, 1H), 4.51 (s, 1H), 4.25-4.16 (m, 2H), 3.75-3.66 (m, 2H), 3.33 (s, 3H), 3.01 (s, 1H), 2.74-2.61 (m, 2H), 2.56 (d, J=12.4 Hz, 1H), 2.15 (s, 1H), 1.71-1.58 (m, 1H).

Ena2: HPLC: 98% purity, 3.070 min. MS: m/z=441.10[M+H]⁺. ¹H NMR (300 MHz, DMSO-d6): 10.02 (d, J=3.4 Hz, 1H), 8.81 (s, 1H), 8.62-8.52 (m, 2H), 8.42-8.33 (m, 2H), 8.22 (d, J=3.5 Hz, 1H), 7.17-7.08 (m, 2H), 5.16-5.06 (m, 1H), 4.49 (dd, J=12.4, 7.6 Hz, 1H), 4.25-4.16 (m, 2H), 3.75-3.66 (m, 2H), 3.33 (s, 3H), 3.01 (s, 2H), 2.71-2.57 (m, 2H), 2.14 (s, 1H), 1.64 (d, J=7.2 Hz, 1H).

### Example 50: 4-(((S)-piperidin-3-yl)amino)-6-(4-(((R)-quinuclidin-3-yl)oxy)phenyl)pyrido[3,2-d]pyrimidine-8-carboxamide

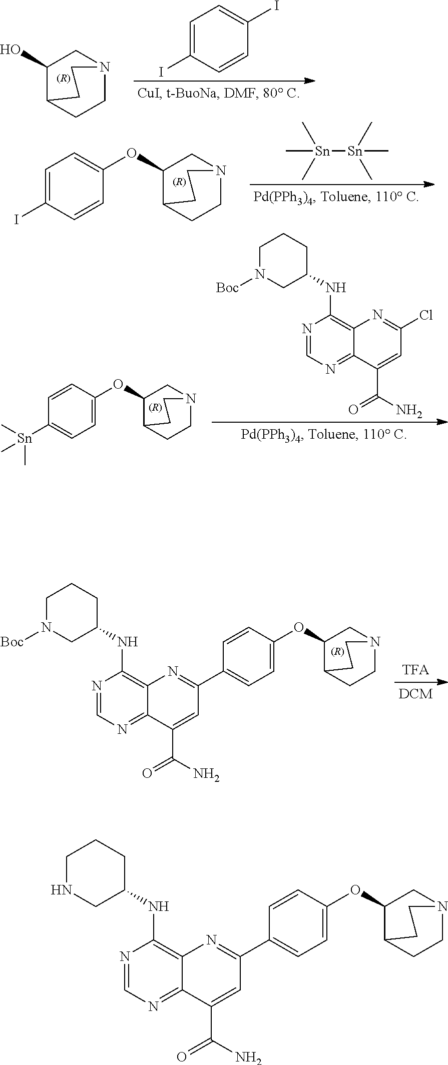

((3R)-3-(4-iodophenoxy)-1-azabicyclo[2.2.2]octane: To a stirred solution of (3R)-1-azabicyclo[2.2.2]octan-3-ol (10 g; 74.69 mmol) and 1,4-diiodobenzene (31.13 g; 89.63 mmol) in dried DMF (200 ml) (with 4 A molecular sieves) were added Sodium tert-butoxide (9.07 g; 89.63 mmol) and Cuprous iodide (1.50 g; 7.47 mmol) in portions at room temperature under N₂ atmosphere. The resulting mixture was stirred for 2 days at 80 degree C. under N₂ atmosphere. The resulting mixture was diluted with water (1000 mL) and extracted with EtOAc (3×400 mL), the combined organic layers were washed with brine (600 mL), dried over anhydrous Na₂SO₄ and concentrated under reduced pressure. The residue was purified by silica gel column eluted with 10% MeOH in DCM to afford (3R)-3-(4-iodophenoxy)-1-azabicyclo[2.2.2]octane as a gray solid (3.00 g; 10.99%)

(3R)-3-[4-(trimethylstannyl)phenoxy]-1-azabicyclo[2.2.2]octane: To a stirred solution of (-3-(4-iodophenoxy)-1-azabicyclo[2.2.2]octane (1 g; 2.74 mmol) and hexamethyldistannane (1.81 g; 5.47 mmol) in Toluene (15 ml) was added Pd(PPh3)4 (333 mg; 0.27 mmol) in portions at room temperature under N₂ atmosphere. The resulting mixture was stirred for overnight at 110 degree C. under N₂ atmosphere. The resulting mixture was diluted with water (15 mL), extracted with EtOAc (3×10 mL). The combined organic layers were washed with brine (15 mL), dried over anhydrous Na₂SO₄. The residue was purified by silica gel column eluted with 10% MeOH in DCM to afford (3R)-3-[4-(trimethylstannyl)phenoxy]-1-azabicyclo[2.2.2]octane as a yellow oil (1.20 g; 93.32%)

tert-butyl (3S)-3-[(6-{4-[(3R)-1-azabicyclo[2.2.2]octan-3-yloxy]phenyl}-8-carbamoylpyrido[3,2-d]pyrimidin-4-yl)amino]piperidine-1-carboxylate: To a stirred solution of (-3-[4-(trimethylstannyl)phenoxy]-1-azabicyclo[2.2.2]octane (689 mg; 1.47 mmol) and tert-butyl (3S)-3-({8-carbamoyl-6-chloropyrido[3,2-d]pyrimidin-4-yl}amino)piperidine-1-carboxylate (200 mg; 0.49 mmol) in DMF (5.00 ml) was added Pd(PPh₃)₄ (58 mg; 0.05 mmol) in portions at room temperature under N₂ atmosphere. The resulting mixture was stirred for 4 h at 100 degree C. under N₂ atmosphere. The resulting mixture was diluted with water (15 mL), extracted with EtOAc (3×10 mL). The combined organic layers were washed with brine (15 mL), dried over anhydrous Na₂SO₄. The residue was purified by silica gel column, eluted with 15% MeOH in DCM to afford crude product. The crude product was purified by Prep-TLC (MeOH:DCM=1:8) to afford tert-butyl (3S)-3-[(6-{4-[(3R)-1-azabicyclo[2.2.2]octan-3-yloxy]phenyl}-8-carbamoylpyrido[3,2-d]pyrimidin-4-yl)amino]piperidine-1-carboxylate as a yellow solid (80 mg; 26.16%)

6-{4-[(3R)-1-azabicyclo[2.2.2]octan-3-yloxy]phenyl}-4-{[(3S)-piperidin-3-yl]amino}pyrido[3,2-d]pyrimidine-8-carboxamide: To a stirred solution of tert-butyl (3S)-3-[(6-{4-[(3R)-1-azabicyclo[2.2.2]octan-3-yloxy]phenyl}-8-carbamoylpyrido[3,2-d]pyrimidin-4-yl)amino]piperidine-1-carboxylate (70 mg; 0.11 mmol) in DCM (3 ml) was added TFA (1 ml) dropwise at room temperature under N₂ atmosphere. The resulting mixture was stirred for 1 h at 25 degree C. under N₂ atmosphere. The reaction was concentrated under reduced pressure. The residue was purified by Prep-HPLC with the following condition Column: XBridge Prep OBD C18 Column, 30*150 mm, 5 μm; Mobile Phase A: Water (10 mmol/l NH₄HCO₃+0.05% NH₄OH), Mobile Phase B: ACN; Flow rate: 60 mL/min; Gradient: 20% B to 50% B in 8 min, 50% B; Wave Length: 254 nm; RT1 (min): 7.0; Number Of Runs: 2 to afford 6-{4-[(3R)-1-azabicyclo[2.2.2]octan-3-yloxy]phenyl}-4-{[(3S)-piperidin-3-yl]amino}pyrido[3,2-d]pyrimidine-8-carboxamide as a yellow solid (7.50 mg; 13.84%)

HPLC: 97% purity, 3.062 min. MS:m/z=474.15[M+H]⁺. ¹H NMR (300 MHz, DMSO-d₆) 10.05 (d, J=3.8 Hz, 1H), 8.77 (s, 1H), 8.53 (s, 1H), 8.40-8.00 (m, 4H), 7.35-6.72 (m, 2H), 4.75-4.46 (m, 1H), 4.40-4.11 (m, 1H), 3.35-3.18 (m, 2H), 3.08 (dd, J=23.1, 9.9 Hz, 1H), 2.91-2.54 (m, 8H), 2.09 q, J=3.4 Hz, 1H), 1.83 (m, J=12.5, 7.5 Hz, 3H), 1.74-1.41 (m, 4H), 1.41-1.27 (m, 1H).

### Example 51: 6-(4-((R)-2-hydroxypropoxy)phenyl)-4-(((S)-piperidin-3-yl)amino)pyrido[3,2-d]pyrimidine-8-carboxamide

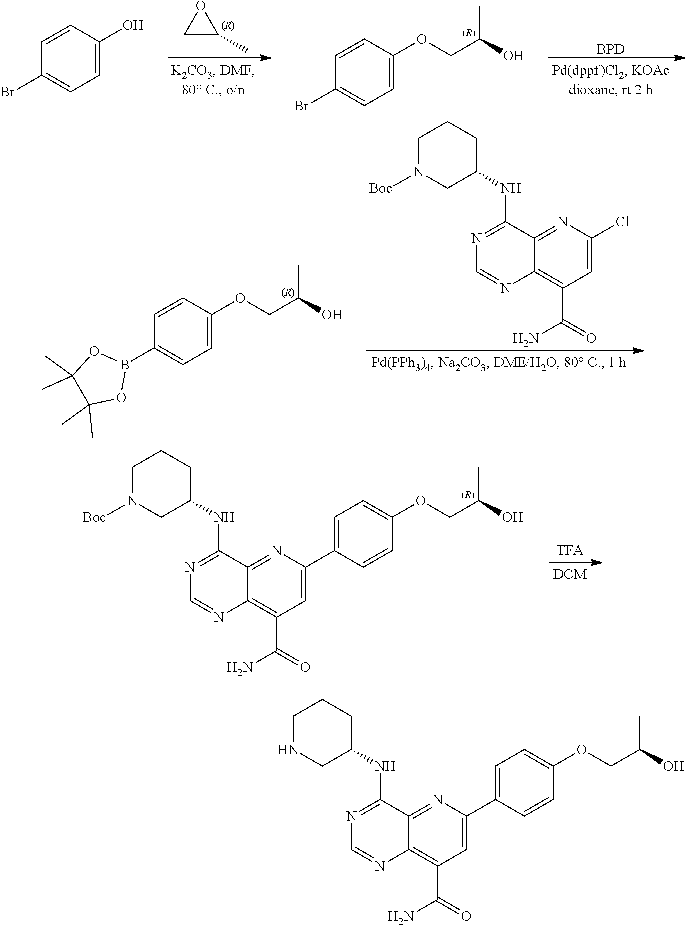

(2R)-1-(4-bromophenoxy)propan-2-ol: To a stirred solution of 4-bromophenol (2 g; 10.98 mmol) and K₂CO₃ (3.20 g; 21.96 mmol) in N,N-dimethylformamide (20 ml) was added (2R)-2-methyloxirane (2.69 g; 43.93 mmol) dropwise. The mixture was stirred for 16 h at 80 degrees. The resulting mixture was diluted with water and extracted with EtOAc. The combined organic layer was washed with brine, dried with anhydrous Na₂SO₄ then filtrated and concentrated under reduced pressure. The crude product was purified by C18 flash column eluted with 56% ACN in water to afford (2R)-1-(4-bromophenoxy)propan-2-ol as a yellow solid (380 mg; 14.97

(2R)-1-[4-(4,4,5,5-tetramethyl-1,3,2-dioxaborolan-2-yl)phenoxy]propan-2-ol: To a stirred solution of (2R)-1-(4-bromophenoxy)propan-2-ol (380 mg; 1.56 mmol) and 4,4,5,5-tetramethyl-2-(4,4,5,5-tetramethyl-1,3,2-dioxaborolan-2-yl)-1,3,2-dioxaborolane (418 mg; 1.56 mmol) in Dioxane-1,4 (5 ml; 58.45 mmol) was added KOAc (307 mg; 3.13 mmol) and Pd(dppf)Cl₂ (121 mg; 0.16 mmol) in portions at r.t. under N₂ atmosphere. The resulting mixture was stirred for 2 h at 100 degrees. The resulting mixture was diluted with water and extracted with EtOAc. The organic layers was washed with brine, dried with Na₂SO₄ and filtrated. The filter was concentrated under reduced pressure. The crude product was purified by C18 flash column to afford (2R)-1-[4-(4,4,5,5-tetramethyl-1,3,2-dioxaborolan-2-yl)phenoxy]propan-2-ol as a yellow solid (300 mg; 69.0%).

tert-butyl (3S)-3-[(8-carbamoyl-6-{4-[(2R)-2-hydroxypropoxy]phenyl}pyrido[3,2-d]pyrimidin-4-yl)amino]piperidine-1-carboxylate: To a solution of tert-butyl (S)-3-((8-carbamoyl-6-chloropyrido[3,2-d]pyrimidin-4-yl)amino)piperidine-1-carboxylate (200 mg; 0.46 mmol) and (2R)-1-[4-(4,4,5,5-tetramethyl-1,3,2-dioxaborolan-2-yl)phenoxy]propan-2-ol (129 mg; 0.46 mmol) in DME (10 ml) and Water (3 ml) were added Na₂CO₃ (162 mg; 1.45 mmol) and Pd(PPh₃)₄ (59 mg; 0.05 mmol). After stirring for 1 h at 80 degrees under a nitrogen atmosphere, the resulting mixture was concentrated under reduced pressure. The residue was purified by silica gel column chromatography, eluted with 70% EA in PE to afford tert-butyl (3S)-3-[(8-carbamoyl-6-{4-[(2R)-2-hydroxypropoxy]phenyl}pyrido[3,2-d]pyrimidin-4-yl)amino]piperidine-1-carboxylate as a yellow solid (200 mg; 76.8%).

6-{4-[(2R)-2-hydroxypropoxy]phenyl}-4-{[(3S)-piperidin-3-yl]amino}pyrido[3,2-d]pyrimidine-8-carboxamide: To a stirred mixture of tert-butyl (3S)-3-[(8-carbamoyl-6-{4-[(2R)-2-hydroxypropoxy]phenyl}pyrido[3,2-d]pyrimidin-4-yl)amino]piperidine-1-carboxylate (180 mg; 0.34 mmol) in DCM (3 ml) was added TFA (1 ml) dropwise at 0 degree. The resulting mixture was stirred for 1 h at room temperature. The residue was purified by Prep-HPLC with the following conditions Column: XBridge Prep OBD C18 Column, 30×150 mm 5 um; Mobile Phase A: Water (10 mmol/l NH₄HCO₃), Mobile Phase B:ACN; Flow rate: 60 mL/min; Gradient: 26 B to 56 B in 9 min; 254 nm; RT1:7) to afford 6-{4-[(2R)-2-hydroxypropoxy]phenyl}-4-{[(3S)-piperidin-3-yl]amino}pyrido[3,2-d]pyrimidine-8-carboxamide as a yellow solid (67 mg; 45.6%).

HPLC 98% purity, 2.89 min, MS: m/s=423.15 [M+H]⁺. ¹H NMR (300 MHz, DMSO-d6): 10.05 (d, J=3.6 Hz, 1H), 8.79 (s, 1H), 8.53 (s, 1H), 8.31 (d, J=8.3 Hz, 3H), 8.20 (d, J=3.6 Hz, 1H), 7.10 (d, J=8.5 Hz, 2H), 4.92 (s, 1H), 4.32-4.24 (m, 1H), 4.02-3.85 (m, 3H), 3.04 (d, J=10.8 Hz, 1H), 2.74 (dd, J=19.5, 10.1 Hz, 2H), 2.57 (d, J=11.4 Hz, 2H), 1.88 (s, 2H), 1.66 (s, 1H), 1.50 (s, 1H), 1.30-1.14 (m, 3H).

### Example 52: (S)-6-(4-(3-morpholinopropoxy)phenyl)-4-(piperidin-3-ylamino)pyrido[3,2-d]pyrimidine-8-carboxamide

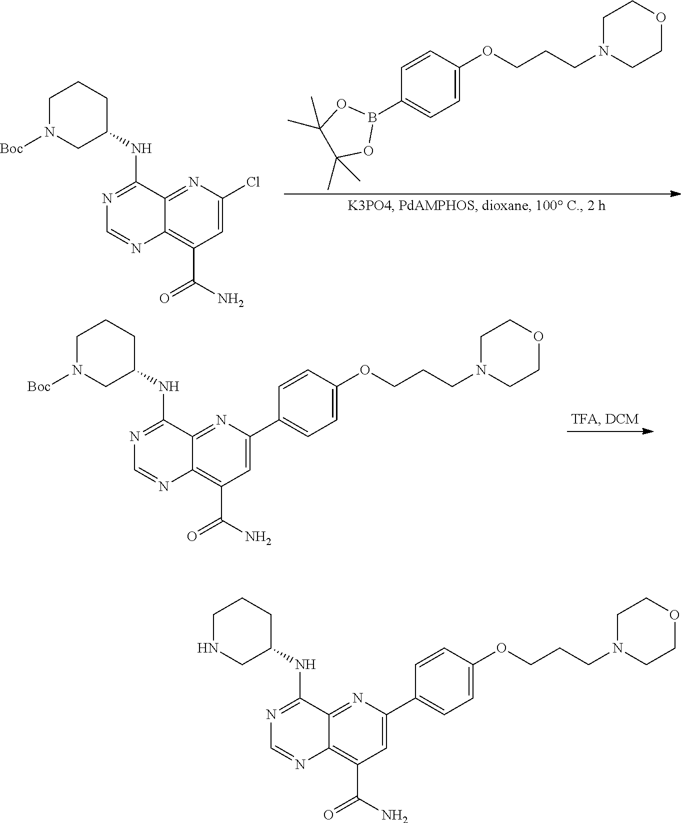 [SMILES: CC(C)(C)OC(=O)N1CCC[C@H](Nc2ncnc3c(C(N)=O)cc(Cl)nc23)C1 ; CC1(C)OB(c2ccc(OCCCN3CCOCC3)cc2)OC1(C)C ; CC(C)(C)OC(=O)N1CCC[C@H](Nc2ncnc3c(C(N)=O)cc(-c4ccc(OCCCN5CCOCC5)cc4)nc23)C1 ; NC(=O)c1cc(-c2ccc(OCCCN3CCOCC3)cc2)nc2c(N[C@H]3CCCNC3)ncnc12]

6-{4-[3-(morpholin-4-yl)propoxy]phenyl}-4-{[(3S)-piperidin-3-yl]amino}pyrido[3,2-d]pyrimidine-8-carboxamide: To a solution of tert-butyl (3S)-3-({8-carbamoyl-6-chloropyrido[3,2-d]pyrimidin-4-yl}amino)piperidine-1-carboxylate (200 mg; 0.47 mmol) and 4-{3-[4-(4,4,5,5-tetramethyl-1,3,2-dioxaborolan-2-yl)phenoxy]propyl}morpholine (258.74 mg; 0.71 mmol) in Dioxane-1,4 (10 ml) and H₂O (2 ml) were added K₃PO₄ (316.31 mg; 1.42 mmol) and Bis(di-tert-butyl(4-dimethylaminophenyl)phosphine)dichloropalladium(II) (35.18 mg; 0.05 mmol). After stirring for 2 h at 80 degrees C. under a nitrogen atmosphere. The residue was purified by silica gel column chromatography, eluted with CH₂Cl₂/MeOH (12:1) to afford 6-{4-[3-(morpholin-4-yl)propoxy]phenyl}-4-{[(3S)-piperidin-3-yl]amino}pyrido[3,2-d]pyrimidine-8-carboxamide as a yellow solid (196 mg; 66.2%).

6-{4-[3-(morpholin-4-yl)propoxy]phenyl}-4-{[(3S)-piperidin-3-yl]amino}pyrido[3,2-d]pyrimidine-8-carboxamide: Into a 50 mL round-bottom flask were added 6-{4-[3-(morpholin-4-yl)propoxy]phenyl}-4-{[(3S)-piperidin-3-yl]amino}pyrido[3,2-d]pyrimidine-8-carboxamide (160 mg; 0.25 mmol) in 6 ml DCM and 3 ml TFA at room temperature. The mixture was stirred for 2 h at room temperature. The residue was purified by Prep-HPLC (Column: XBridge Prep OBD C18 Column, 30*150 mm, 5 μm; Mobile Phase A: Water (10 mmol/l NH₄HCO₃+0.05% NH₄OH), Mobile Phase B: ACN; Flow rate: 60 mL/min; Gradient: 20% B to 50% B in 9 min, 50% B; Wave Length: 254 nm; RT1 (min): 7.2; Number Of Runs: 2) to afford 6-{4-[3-(morpholin-4-yl)propoxy]phenyl}-4-{[(3S)-piperidin-3-yl]amino}pyrido[3,2-d]pyrimidine-8-carboxamide as a yellow solid (73.7 mg; 55.9%).

HPLC 95% purity, 2.26 min, MS: m/s=492.30 [M+H]⁺. ¹H NMR (300 MHz, DMSO-d6): 10.04 (d, 1H), 8.77 (s, 1H), 8.52 (s, 1H), 8.30 (dd, 3H), 8.20 (d, 1H), 7.14-7.02 (m, 2H), 4.27 (dt, 1H), 4.10 (t, 2H), 3.57 (t, 4H), 3.02 (dd, 1H), 2.78 (s, 2H), 2.71 (dd, 1H), 2.56 (d, 2H), 2.48-2.32 (m, 5H), 1.98-1.75 (m, 4H), 1.67 (d, 1H), 1.47 (dd, 1H).

### Example 53: 6-(4-((S)-1-morpholinoethyl)phenyl)-4-(((S)-piperidin-3-yl)amino)pyrido[3,2-d]pyrimidine-8-carboxamide

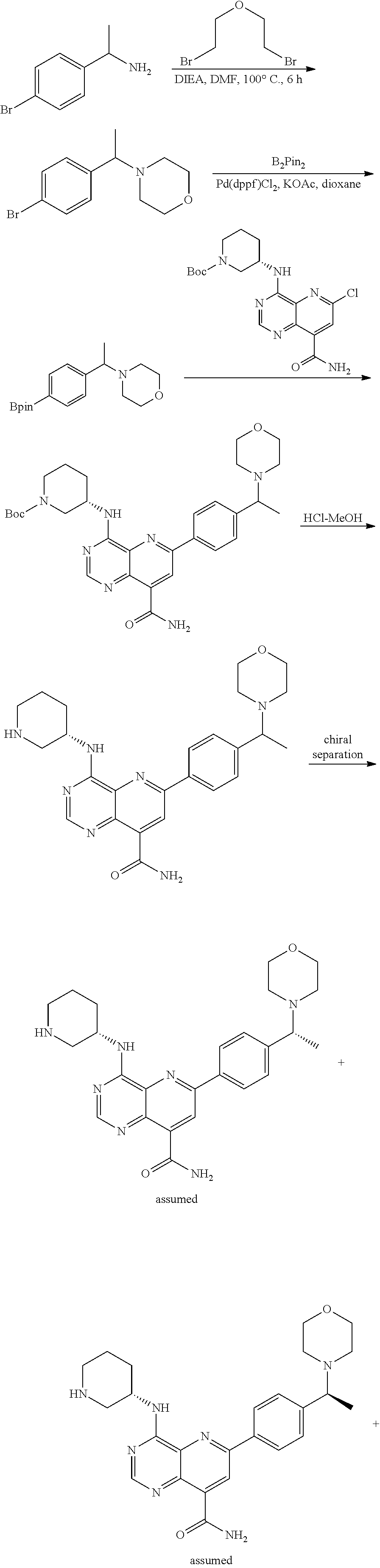

4-[1-(4-bromophenyl)ethyl]morpholine: To a stirred solution of 1-(4-bromophenyl)ethanamine (3 g, 14.244 mmol) and 1-bromo-2-(2-bromoethoxy)ethane (4.17 g, 17.093 mmol) in DMF (30 mL, 389.854 mmol) was added DIEA (5.81 g, 42.706 mmol). The resulting mixture was stirred for overnight at 100° C. under nitrogen atmosphere. The reaction was quenched with Water at room temperature. The resulting mixture was extracted with EtOAc (3×100 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc (3:7) to afford 4-[1-(4-bromophenyl)ethyl]morpholine as a yellow oil (3.3 g, 80.18%).

4-[1-[4-(4,4,5,5-tetramethyl-1,3,2-dioxaborolan-2-yl)phenyl]ethyl]morpholine: To a solution of 4-[1-(4-bromophenyl)ethyl]morpholine (2000 mg, 6.922 mmol) and BPD (2220.20 mg, 8.306 mmol) in dioxane (20 mL, 227.014 mmol) were added KOAc (2145.15 mg, 20.765 mmol) and Pd(dppf)Cl₂ in CH₂Cl₂ (594.99 mg, 0.692 mmol). After stirring for 2 h at 100° C. under a nitrogen atmosphere, the resulting mixture was concentrated under reduced pressure. The residue was purified by silica gel column chromatography, eluted with PE/EtOAc (5:1) to afford 4-[1-[4-(4,4,5,5-tetramethyl-1,3,2-dioxaborolan-2-yl)phenyl]ethyl]morpholine as a yellow oil (2.1 g, 95.54%).

(3S)-3-[(8-carbamoyl-6-[4-[1-(morpholin-4-yl)ethyl]phenyl]pyrido[3,2-d]pyrimidin-4-yl)amino]piperidine-1-carboxylate: To a solution of tert-butyl (3S)-3-([8-carbamoyl-6-chloropyrido[3,2-d]pyrimidin-4-yl]amino)piperidine-1-carboxylate (200 mg, 0.346 mmol) and 4-[1-[4-(4,4,5,5-tetramethyl-1,3,2-dioxaborolan-2-yl)phenyl]ethyl]morpholine (140 mg, 0.441 mmol) in 1,4-dioxane (5 mL, 59.020 mmol) and H₂O (1 mL, 55.508 mmol) were added K₃PO₄ (232 mg, 1.038 mmol) and Pd(Amphos)Cl₂ (21 mg, 0.028 mmol). The resulting mixture stirring for 2 h at 100° C. under a nitrogen atmosphere, the resulting mixture was concentrated under reduced pressure. The residue was purified by silica gel column chromatography, eluted with CH₂Cl₂/MeCOH (95:5) to afford tert-butyl (3S)-3-[(8-carbamoyl-6-[4-[1-(morpholin-4-yl)ethyl]phenyl]pyrido[3,2-d]pyrimidin-4-yl)amino]piperidine-1-carboxylate as a yellow solid (210 mg, 88.28%).

6-{4-[1-(morpholin-4-yl)ethyl]phenyl}-4-{[(3S)-piperidin-3-yl]amino}pyrido[3,2-d]pyrimidine-8-carboxamide: A solution of tert-butyl (3S)-3-[(8-carbamoyl-6-[4-[1-(morpholin-4-yl)ethyl]phenyl]pyrido[3,2-d]pyrimidin-4-yl)amino]piperidine-1-carboxylate (200 mg, 0.291 mmol) in HCl (gas) in 1,4-dioxane (2 mL, 4.204 mmol, 14.47 equiv, 12%) was stirred for 1 h at room temperature under nitrogen atmosphere. The resulting mixture was concentrated under vacuum. The crude product (210 mg) was purified by Prep-HPLC with the following conditions (2#SHIMADZU (HPLC-01)): Column, XBridge Prep OBD C18 Column, 30*150 mm 5 um; mobile phase, Water (10 mmol/l NH₄HCO₃+0.05% NH₄OH) and ACN (20% Phase B up to 50% in 8 min); Detector, uv 254. product was obtained. This resulted in 6-[4-[1-(morpholin-4-yl)ethyl]phenyl]-4-[(3S)-piperidin-3-ylamino]pyrido[3,2-d]pyrimidine-8-carboxamide (130 mg, 96.64%) as a light yellow solid. This was separated by chiral prep-HPLC and two single isomers were obtained, 6-{4-[(1S)-1-(morpholin-4-yl)ethyl]phenyl}-4-{[(3S)-piperidin-3-yl]amino}pyrido[3,2-d]pyrimidine-8-carboxamide (45.30 mg; 31.4%) as light yellow solid, and 6-{4-[(1R)-1-(morpholin-4-yl)ethyl]phenyl}-4-{[(3S)-piperidin-3-yl]amino}pyrido[3,2-d]pyrimidine-8-carboxamide as light yellow solid (49.90 mg; 34.7

HPLC isomer 1: 96% purity, 2.18 min, MS: m/s=462.20 [M+H]⁺. ¹H NMR (300 MHz, DMSO, ppm) 10.05 (d, J=3.6 Hz, 1H), 8.82 (s, 1H), 8.56 (s, 1H), 8.35 (d, J=8.6 Hz, 1H), 8.25 (dd, J=15.8, 5.9 Hz, 3H), 7.49 (d, J=8.1 Hz, 2H), 4.30 (dd, J=8.8, 4.4 Hz, 1H), 3.57 (t, J=4.7 Hz, 4H), 3.44 (q, J=6.6 Hz, 1H), 3.05 (dd, J=11.7, 3.7 Hz, 1H), 2.86-2.68 (m, 2H), 2.65-2.51 (m, 1H), 2.42 (q, J=5.5, 4.4 Hz, 2H), 2.30 (dt, J=10.9, 4.6 Hz, 2H), 1.89 (s, 2H), 1.68 (d, J=12.1 Hz, 1H), 1.57-1.44 (m, 1H), 1.32 (d, J=6.6 Hz, 3H).

HPLC isomer 2: 97% purity, 2.17 min, MS: m/s=462.20 [M+H]⁺. ¹H NMR (300 MHz, DMSO, ppm) 10.05 (d, J=3.6 Hz, 1H), 8.82 (s, 1H), 8.56 (s, 1H), 8.35 (d, J=8.6 Hz, 1H), 8.25 (dd, J=15.8, 5.9 Hz, 3H), 7.49 (d, J=8.1 Hz, 2H), 4.30 (dd, J=8.8, 4.4 Hz, 1H), 3.57 (t, J=4.7 Hz, 4H), 3.44 (q, J=6.6 Hz, 1H), 3.05 (dd, J=11.7, 3.7 Hz, 1H), 2.86-2.68 (m, 2H), 2.65-2.51 (m, 1H), 2.42 (q, J=5.5, 4.4 Hz, 2H), 2.30 (dt, J=10.9, 4.6 Hz, 2H), 1.89 (s, 2H), 1.68 (d, J=12.1 Hz, 1H), 1.57-1.44 (m, 1H), 1.32 (d, J=6.6 Hz, 3H).

### Example 54: (S)-6-(4-((methylsulfonyl)methyl)phenyl)-4-(piperidin-3-ylamino)pyrido[3,2-d]pyrimidine-8-carboxamide

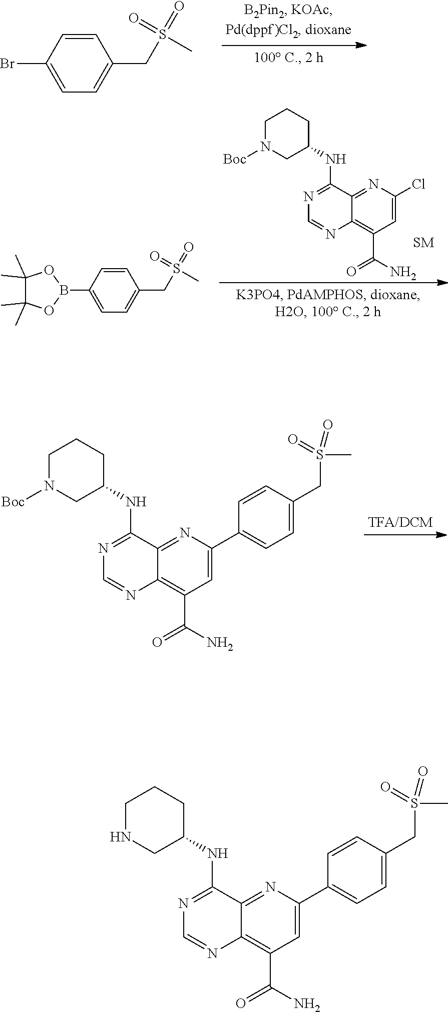

2-[4-(methanesulfonylmethyl)phenyl]-4,4,5,5-tetramethyl-1,3,2-dioxaborolane: To a solution of 1-bromo-4-(methanesulfonylmethyl)benzene (300 mg, 1.144 mmol) and BPD (612 mg, 2.290 mmol) in dioxane (30 mL, 340.502 mmol) were added Pd(dppf)Cl₂ (88 mg, 0.114 mmol) and KOAc (355 mg, 3.436 mmol). After stirring for 3 h at 100° C. under a nitrogen atmosphere. The residue was purified by silica gel column chromatography, eluted with CH₂Cl₂/MeOH (9:1) to afford 2-[4-(methanesulfonylmethyl)phenyl]-4,4,5,5-tetramethyl-1,3,2-dioxaborolane as a yellow oil (310 mg, 77.76%).

tert-butyl (3S)-3-([8-carbamoyl-6-[4-(methanesulfonylmethyl)phenyl]pyrido[3,2-d]pyrimidin-4-yl]amino)piperidine-1-carboxylate: To a solution of tert-butyl (3S)-3-([8-carbamoyl-6-chloropyrido[3,2-d]pyrimidin-4-yl]amino)piperidine-1-carboxylate (150 mg, 0.332 mmol) and 2-[4-(methanesulfonylmethyl)phenyl]-4,4,5,5-tetramethyl-1,3,2-dioxaborolane (231 mg, 0.664 mmol) in dioxane (10 mL, 118.041 mmol) and H₂O (2 mL, 111.017 mmol) were added Pd(Amphos)Cl₂ (25 mg, 0.033 mmol) and K₃PO₄ (222 mg, 0.995 mmol). After stirring for 3 h at 100° C. under a nitrogen atmosphere. The residue was purified by silica gel column chromatography, eluted with CH₂Cl₂/MeOH (8:1) to afford tert-butyl (3S)-3-([8-carbamoyl-6-[4-(methanesulfonylmethyl)phenyl]pyrido[3,2-d]pyrimidin-4-yl]amino)piperidine-1-carboxylate as a yellow solid (155 mg, 86.41%).

6-[4-(methanesulfonylmethyl)phenyl]-4-[(3S)-piperidin-3-ylamino]pyrido[3,2-d]pyrimidine-8-carboxamide: Into a 10 mL vial were added tert-butyl (3S)-3-([8-carbamoyl-6-[4-(methanesulfonylmethyl)phenyl]pyrido[3,2-d]pyrimidin-4-yl]amino)piperidine-1-carboxylate (150 mg, 0.277 mmol) in TFA (5 mL, 67.315 mmol) and DCM (10 mL, 157.300 mmol) at room temperature. The mixture was stirred for 2 h at room temperature. The residue was purified by Prep-HPLC (Column: XBridge Prep OBD C18 Column, 30*150 mm, 5 μm; Mobile Phase A: Water (10 mmol/I NH₄HCO₃+0.05% NH₄OH), Mobile Phase B: ACN; Flow rate: 60 mL/min; Gradient: 24% B to 54% B in 8 min, 54% B; Wave Length: 254 nm; RT1 (min): 6.48; Number Of Runs: 2) to afford 6-[4-(methanesulfonylmethyl)phenyl]-4-[(3S)-piperidin-3-ylamino]pyrido[3,2-d]pyrimidine-8-carboxamide as a yellow solid (54.1 mg, 43.38%).

HPLC: 98% purity, 2.170 min, MS: m/s=441.0 [M+H]⁺. ¹H NMR (300 MHz, DMSO-d6): 10.04 (d, 1H), 8.87 (s, 1H), 8.58 (s, 1H), 8.40 (dd, 3H), 8.24 (d, 1H), 7.62 (d, 2H), 4.61 (s, 2H), 4.34-4.25 (m, 1H), 3.04 (d, 1H), 2.95 (s, 3H), 2.87-2.66 (m, 2H), 2.57 (d, 1H), 1.89 (s, 2H), 1.67 (s, 1H), 1.51 (s, 1H).

### Example 55: (S)-6-(6-(2-methoxyethoxy)pyridin-3-yl)-4-(piperidin-3-ylamino)pyrido[3,2-d]pyrimidine-8-carboxamide

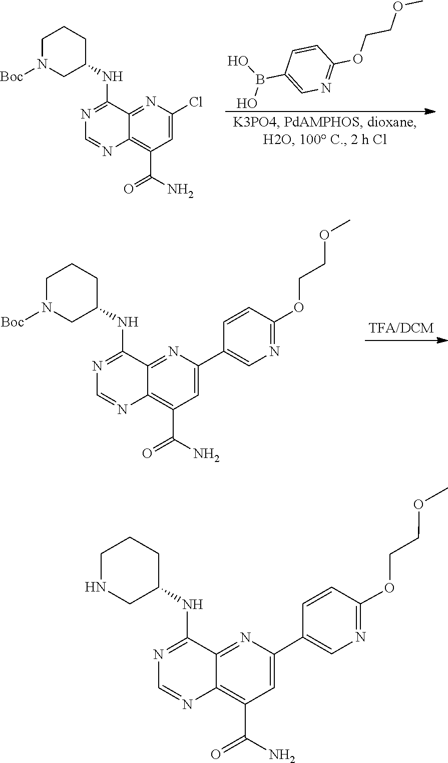 [SMILES: CC(C)(C)OC(=O)N1CCC[C@H](Nc2ncnc3c(C(N)=O)cc(Cl)nc23)C1 ; COCCOc1ccc(B(O)O)cn1 ; COCCOc1ccc(-c2cc(C(N)=O)c3ncnc(N[C@H]4CCCN(C(=O)OC(C)(C)C)C4)c3n2)cn1 ; COCCOc1ccc(-c2cc(C(N)=O)c3ncnc(N[C@H]4CCCNC4)c3n2)cn1]

tert-butyl (3S)-3-([8-carbamoyl-6-[6-(2-methoxyethoxy)pyridin-3-yl]pyrido[3,2-d]pyrimidin-4-yl]amino)piperidine-1-carboxylate: To a solution of tert-butyl (3S)-3-([8-carbamoyl-6-chloropyrido[3,2-d]pyrimidin-4-yl]amino)piperidine-1-carboxylate (200 mg, 0.444 mmol) and 6-(2-methoxyethoxy)pyridin-3-ylboronic acid (139 mg, 0.670 mmol) in dioxane (10 mL, 118.041 mmol) and H₂O (2 mL, 111.017 mmol) were added Pd(Amphos)Cl₂ (34 mg, 0.046 mmol) and K₃PO₄ (298 mg, 1.334 mmol). After stirring for 2 h at 100° C. under a nitrogen atmosphere. After stirring for 2 h at 100° C. under a nitrogen atmosphere. The residue was purified by silica gel column chromatography, eluted with PE/EtOAc (1:99) to afford tert-butyl (3S)-3-([8-carbamoyl-6-[6-(2-methoxyethoxy)pyridin-3-yl]pyrido[3,2-d]pyrimidin-4-yl]amino)piperidine-1-carboxylate as a yellow solid (156 mg, 65.24%).

6-[6-(2-methoxyethoxy)pyridin-3-yl]-4-[(3S)-piperidin-3-ylamino]pyrido[3,2-d]pyrimidine-8-carboxamide: To a solution of tert-butyl (3S)-3-([8-carbamoyl-6-[6-(2-methoxyethoxy)pyridin-3-yl]pyrido[3,2-d]pyrimidin-4-yl]amino)piperidine-1-carboxylate (145 mg, 0.269 mmol) and TFA (5 mL, 63.949 mmol) in DCM (15.00 mL, 235.951 mmol). After stirring for 1 h at room temperature. The residue was purified by Prep-HPLC (Column: Atlantis HILIC OBD Column, 19*150 mm*5 μm; Mobile Phase A: Water (10 mmol/l NH₄HCO₃+0.05% NH₄OH), Mobile Phase B: ACN; Flow rate: 60 mL/min; Gradient: 17% B to 47% B in 8 min, 47% B; Wave Length: 254 nm; RT1 (min): 6.2; Number Of Runs: 2) to afford 6-[6-(2-methoxyethoxy)pyridin-3-yl]-4-[(3S)-piperidin-3-ylamino]pyrido[3,2-d]pyrimidine-8-carboxamide as a yellow solid (78.1 mg, 67.83%).

HPLC: 99% purity, 2.446 min, MS: m/s=424.2 [M+H]⁺. ¹H NMR (300 MHz, DMSO-d₆, ppm): 10.00 (d, J=3.6 Hz, 1H), 9.20 (d, J=2.5 Hz, 1H), 8.79 (s, 1H), 8.67 (dd, J=8.8, 2.6 Hz, 1H), 8.54 (s, 1H), 8.41 (d, J=8.7 Hz, 1H), 8.21 (d, J=3.7 Hz, 1H), 6.98 (d, J=8.7 Hz, 1H), 4.52-4.43 (m, 3H), 3.75-3.66 (m, 2H), 3.32 (s, 3H), 3.04 (dd, J=11.6, 3.8 Hz, 1H), 2.83 (d, J=12.0 Hz, 1H), 2.71 (dd, J=11.7, 9.0 Hz, 1H), 1.90 (s, 2H), 1.76 (dd, J=21.6, 8.7 Hz, 1H), 1.72-1.63 (m, 1H), 1.48 (d, J=11.6 Hz, 1H).

### Example 56: (S)-6-(4-((3-fluoro-3-methylazetidin-1-yl)methyl)phenyl)-4-(piperidin-3-ylamino)pyrido[3,2-d]pyrimidine-8-carboxamide

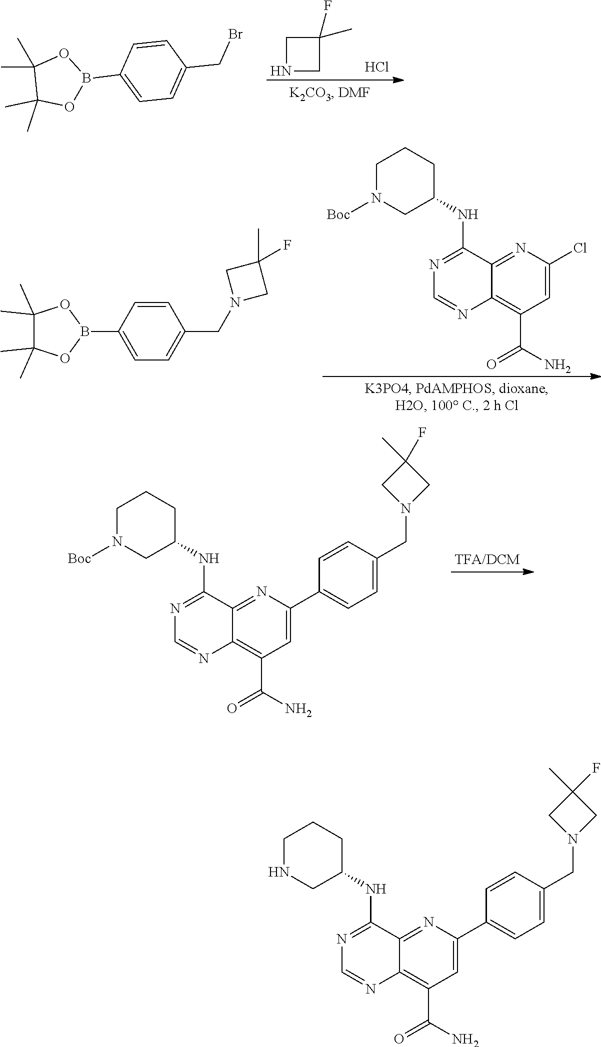

3-fluoro-3-methyl-1-[[4-(4,4,5,5-tetramethyl-1,3,2-dioxaborolan-2-yl)phenyl]methyl]azetidine: A solution of 2-[4-(bromomethyl)phenyl]-4,4,5,5-tetramethyl-1,3,2-dioxaborolane (1 g, 3.199 mmol), 3-fluoro-3-methylazetidine hydrochloride (0.50 g, 3.783 mmol) and K₂CO₃ (2.78 g, 19.109 mmol) in CAN (5.00 mL, 95.123 mmol) was stirred for 3 h at 50° C. The resulting mixture was filtered, the cake was washed with ACN. The filtrate was concentrated under reduced pressure. This resulted in 3-fluoro-3-methyl-1-[[4-(4,4,5,5-tetramethyl-1,3,2-dioxaborolan-2-yl)phenyl]methyl]azetidine as a off-white oil (1.2 g, 86.05%).

tert-butyl (3S)-3-[(8-carbamoyl-6-[4-[(3-fluoro-3-methylazetidin-1-yl)methyl]phenyl]pyrido[3,2-d]pyrimidin-4-yl)amino]piperidine-1-carboxylate: To a solution of tert-butyl (3S)-3-([8-carbamoyl-6-chloropyrido[3,2-d]pyrimidin-4-yl]amino)piperidine-1-carboxylate (150 mg, 0.332 mmol) and 3-fluoro-3-methyl-1-[[4-(4,4,5,5-tetramethyl-1,3,2-dioxaborolan-2-yl)phenyl]methyl]azetidine (289 mg, 0.663 mmol) in dioxane (10 mL, 118.041 mmol) and H₂O (2 mL, 111.017 mmol) were added Pd(Amphos)Cl₂ (25 mg, 0.034 mmol) and K₃PO₄ (222 mg, 0.994 mmol). After stirring for 3 h at 100° C. under a nitrogen atmosphere. The residue was purified by silica gel column chromatography, eluted with CH₂Cl₂/MeOH (8:1) to afford tert-butyl (3S)-3-[(8-carbamoyl-6-[4-[(3-fluoro-3-methylazetidin-1-yl)methyl]phenyl]pyrido[3,2-d]pyrimidin-4-yl)amino]piperidine-1-carboxylate as a yellow oil (185 mg, 77.40%).

6-[4-[(3-fluoro-3-methylazetidin-1-yl)methyl]phenyl]-4-[(3S)-piperidin-3-ylamino]pyrido[3,2-d]pyrimidine-8-carboxamide: Into a 50 mL round-bottom flask were added tert-butyl (3S)-3-[(8-carbamoyl-6-[4-[(3-fluoro-3-methylazetidin-1-yl)methyl]phenyl]pyrido[3,2-d]pyrimidin-4-yl)amino]piperidine-1-carboxylate (180 mg, 0.250 mmol) in TFA (1 mL, 63.949 mmol) and DCM (5 mL, 78.650 mmol) at room temperature. The mixture was stirred for 2 h at room temperature under argon atmosphere. The residue was purified by Prep-HPLC (Column: XBridge Prep OBD C18 Column, 30*150 mm, 5 μm; Mobile Phase A: Water (10 mmol/I NH₄HCO₃+0.05% NH₄OH), Mobile Phase B: ACN; Flow rate: 60 mL/min; Gradient: 23% B to 53% B in 8 min, 53% B; Wave Length: 254 nm; RT1 (min): 6.4; Number Of Runs: 2) to afford 6-[4-[(3-fluoro-3-methylazetidin-1-yl)methyl]phenyl]-4-[(3S)-piperidin-3-ylamino]pyrido[3,2-d]pyrimidine-8-carboxamide as off-white solid (36.7 mg, 31.99%).

HPLC: 98% purity, 1.975 min, MS: m/s=450.3 [M+H]⁺. ¹H NMR (300 MHz, DMSO-d6): 10.04 (d, 1H), 8.82 (s, 1H), 8.56 (s, 1H), 8.35 (d, 1H), 8.29 (d, 2H), 8.22 (d, 1H), 7.47 (d, 2H), 4.33-4.24 (m, 1H), 3.34 (d, 4H), 3.34-3.23 (m, 2H), 3.20 (d, 1H), 3.09-2.98 (m, 1H), 2.79 (s, 1H), 2.73 (dd, 1H), 1.88 (s, 2H), 1.66 (s, 1H), 1.58 (s, 1H), 1.51 (s, 2H), 1.47 (s, 1H).

### Example 57: 4-[(3S)-piperidin-3-ylamino]-6-(4-[[(2S)-2(trifluoromethyl) morpholin-4-yl]methyl]phenyl) pyrido [3,2-d]pyrimidine-8-carboxamide

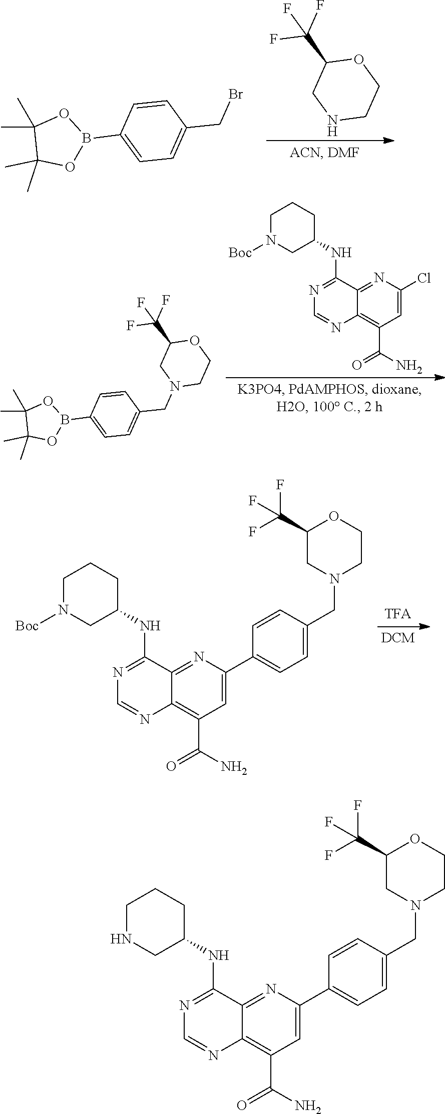 [SMILES: CC1(C)OB(c2ccc(CBr)cc2)OC1(C)C ; FC(F)(F)[C@@H]1CNCCO1 ; CC1(C)OB(c2ccc(CN3CCO[C@H](C(F)(F)F)C3)cc2)OC1(C)C ; CC(C)(C)OC(=O)N1CCC[C@H](Nc2ncnc3c(C(N)=O)cc(Cl)nc23)C1 ; CC(C)(C)OC(=O)N1CCC[C@H](Nc2ncnc3c(C(N)=O)cc(-c4ccc(CN5CCO[C@H](C(F)(F)F)C5)cc4)nc23)C1 ; NC(=O)c1cc(-c2ccc(CN3CCO[C@H](C(F)(F)F)C3)cc2)nc2c(N[C@H]3CCCNC3)ncnc12]

(2S)-4-[[4-(4,4,5,5-tetramethyl-1,3,2-dioxaborolan-2-yl)phenyl]methyl]-2-(trifluoromethyl)morpholine: A solution of 2-[4-(bromomethyl)phenyl]-4,4,5,5-tetramethyl-1,3,2-dioxaborolane (500 mg, 1.650 mmol), (2S)-2-(trifluoromethyl)morpholine hydrochloride (400 mg, 1.984 mmol,) and K₂CO₃ (480 mg, 3.299 mmol) in ACN (10.00 mL) was stirred for 3 h at 50° C. The resulting mixture was filtered, the filter cake was washed with ACN. The filtrate was concentrated under reduced pressure. This resulted in (2S)-4-[[4-(4,4,5,5-tetramethyl-1,3,2-dioxaborolan-2-yl)phenyl]methyl]-2-(trifluoromethyl)morpholine (660 mg, 68.76%) as a white oil.

tert-butyl (3S)-3-[[8-carbamoyl-6-(4-[[(2S)-2-(trifluoromethyl) morpholin-4-yl]methyl]phenyl) pyrido[3,2-d]pyrimidin-4-yl]amino]piperidine-1-carboxylate: To a solution of tert-butyl (3S)-3-([8-carbamoyl-6-chloropyrido[3,2-d]pyrimidin-4-yl]amino) piperidine-1-carboxylate (150 mg, 0.333 mmol) and (2S)-4-[[4-(4,4,5,5-tetramethyl-1,3,2-dioxaborolan-2-yl) phenyl]methyl]-2-(trifluoromethyl) morpholine (264 mg, 0.498 mmol) in dioxane (10 mL) and H₂O (2 mL) were added Pd(AMPHOS)Cl₂ (26 mg, 0.035 mmol) and K₃PO₄ (223 mg, 0.998 mmol). After stirring for 2 h at 100° C. under a nitrogen atmosphere. The residue was purified by silica gel column chromatography, eluted with CH₂Cl₂/MeOH (12:1) to afford tert-butyl (3S)-3-[[8-carbamoyl-6-(4-[[(2S)-2-(trifluoromethyl) morpholin-4-yl]methyl]phenyl) pyrido[3,2-d]pyrimidin-4-yl]amino]piperidine-1-carboxylate (168 mg, 73.28%) as a yellow solid.

4-[(3S)-piperidin-3-ylamino]-6-(4-[[(2S)-2-(trifluoromethyl) morpholin-4-yl]methyl]phenyl) pyrido[3,2-d]pyrimidine-8-carboxamide: To a solution of tert-butyl (3S)-3-[[8-carbamoyl-6-(4-[[(2S)-2-(trifluoromethyl) morpholin-4-yl]methyl]phenyl)pyrido[3,2-d]pyrimidin-4-yl]amino]piperidine-1-carboxylate (155 mg, 0.225 mmol) and TFA (5.00 mL, 67.315 mmol) in DCM (15.00 mL). After stirring for 1 h at room temperature. The crude product was purified by Prep-HPLC with following conditions Column: XBridge Prep OBD C18 Column, 30*150 mm, 5 μm; Mobile Phase A: Water (10 mmol/l NH₄HCO₃+0.05% NH₄OH), Mobile Phase B: ACN; Flow rate: 60 mL/min; Gradient: 40% B to 70% B in 8 min, 70% B; Wave Length: 254 nm; RT1 (min): 6.8) to afford 4-[(3S)-piperidin-3-ylamino]-6-(4-[[(2S)-2-(trifluoromethyl)morpholin-4-yl]methyl]phenyl)pyrido[3,2-d]pyrimidine-8-carboxamide (65.9 mg, 55.26%) as a white solid.

HPLC: 97% purity, RT=2.449 min. MS: m/z=516.2 [M+H]⁺. ¹H NMR (300 MHz, DMSO-d6): 10.01-9.94 (m, 1H), 8.87 (s, 1H), 8.62 (s, 1H), 8.56 (d, J=8.6 Hz, 1H), 8.38 (d, J=8.1 Hz, 2H), 8.26 (d, J=3.7 Hz, 1H), 7.53 (d, J=8.1 Hz, 2H), 4.63 (d, J=10.1 Hz, 1H), 4.19 (t, J=7.4 Hz, 1H), 3.95 (d, J=11.2 Hz, 1H), 3.68 (s, 2H), 3.64 (d, J=10.8 Hz, 1H), 3.20 (d, J=12.1 Hz, 1H), 3.08 (t, J=11.2 Hz, 1H), 2.91 (d, J=11.0 Hz, 1H), 2.75 (dd, J=23.0, 11.6 Hz, 2H), 2.29-2.08 (m, 2H), 2.00 (s, 2H), 1.83 (dt, J=40.9, 12.3 Hz, 3H).

### Example 58: 6-[4-(1-hydroxycyclopropyl) phenyl]-4-[(3S)-piperidin-3-ylamino]pyrido[3,2-d]pyrimidine-8-carboxamide

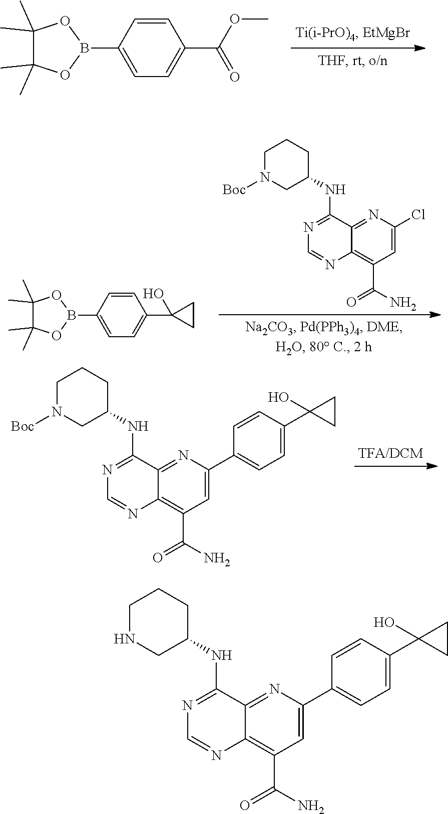

1-[4-(4,4,5,5-tetramethyl-1,3,2-dioxaborolan-2-yl) phenyl]cyclopropan-1-ol: To a stirred solution methyl 4-(tetramethyl-1,3,2-dioxaborolan-2-yl) benzoate (5.00 g, 18.122 mmol) in THF (50.00 mL) were added Ti(i-PrO)₄ (5.42 g, 18.117 mmol,) and EtMgBr (17.20 mL, 26.070 mmol) dropwise at 0° C. under nitrogen atmosphere. The resulting mixture was stirred for overnight at room temperature under nitrogen atmosphere. The resulting mixture was diluted with water (50 mL). The resulting mixture was extracted with EtOAc (3*50 mL). The combined organic layers were washed with brine (1*100 mL), dried over anhydrous Na₂SO₄. After filtration, the filtrate was concentrated under reduced pressure. The residue was purified by silica gel column chromatography, eluted with 14% EtOAc in PE to afford 1-[4-(4,4,5,5-tetramethyl-1,3,2-dioxaborolan-2-yl) phenyl]cyclopropan-1-ol (3.86 g, 72.38%) as a white solid.

tert-butyl (3S)-3-([8-carbamoyl-6-[4-(1-hydroxycyclopropyl)phenyl]pyrido[3,2-d]pyrimidin-4-yl]amino)piperidine-1-carboxylate: To a stirred solution tert-butyl (3S)-3-([8-carbamoyl-6-chloropyrido[3,2-d]pyrimidin-4-yl]amino)piperidine-1-carboxylate (250 mg, 0.573 mmol) and 1-[4-(4,4,5,5-tetramethyl-1,3,2-dioxaborolan-2-yl)phenyl]cyclopropan-1-ol (321 mg, 1.091 mmol,) in DME (2.40 mL) and H₂O (0.80 mL) were added Na₂CO₃ (262 mg, 2.348 mmol) and Pd(PPh₃)₄ (72 mg, 0.059 mmol) in portions at room temperature under nitrogen atmosphere. The resulting mixture was stirred for 2 h at 80° C. under nitrogen atmosphere. The residue was purified by silica gel column chromatography, eluted with 45% EtOAc in PE. The residue was purified by silica gel column chromatography, eluted with 25% EtOAc in DCM to afford tert-butyl (3S)-3-([8-carbamoyl-6-[4-(1-hydroxycyclopropyl) phenyl]pyrido[3,2-d]pyrimidin-4-yl]amino) piperidine-1-carboxylate (200 mg, 47.12%) as a yellow solid.

6-[4-(1-hydroxycyclopropyl) phenyl]-4-[(3S)-piperidin-3-ylamino]pyrido[3,2-d]pyrimidine-8-carboxamide: To a stirred solution tert-butyl (3S)-3-([8-carbamoyl-6-[4-(1-hydroxycyclopropyl) phenyl]pyrido[3,2-d]pyrimidin-4-yl]amino)piperidine-1-carboxylate (200 mg, 0.270 mmol) in DCM (3.00 mL) was added TFA (1.00 mL) dropwise at 0° C. under nitrogen atmosphere. The resulting mixture was stirred for 1 h at room temperature under nitrogen atmosphere. The crude product was purified by Prep-HPLC with the following conditions (2#SHIMADZU (HPLC-01)): Column, Xselect CSH OBD Column 30*150 mm Sum, n; mobile phase, Water (0.1% FA) and ACN (5% Phase B up to 30% in 8 min); Detector, uv254 to afford 6-[4-(1-hydroxycyclopropyl)phenyl]-4-[(3S)-piperidin-3-ylamino]pyrido[3,2-d]pyrimidine-8-carboxamide (12.7 mg, 10.61%) as a yellow solid and 4-[(3S)-piperidin-3-ylamino]-6-(4-propanoylphenyl)pyrido[3,2-d]pyrimidine-8-carboxamide; formic acid salt (19.5 mg, 15.51%) as a yellow solid.

6-[4-(1-hydroxycyclopropyl) phenyl]-4-[(3S)-piperidin-3-ylamino]pyrido[3,2-d]pyrimidine-8-carboxamide: HPLC: 91% purity, RT=2.360 min. MS: m/z=405.2 [M+H]^{+ 1}H NMR (300 MHz, DMSO-d6): 10.03 (d, J=3.7 Hz, 1H), 8.85 (s, 1H), 8.59 (s, 1H), 8.49 (d, J=8.6 Hz, 1H), 8.42 (s, 1H), 8.32 (d, J=8.1 Hz, 1H), 8.25 (d, J=3.7 Hz, 1H), 7.42 (d, J=8.2 Hz, 2H), 4.47 (s, 1H), 3.20 (d, J=12.1 Hz, 1H), 2.98 (s, 2H), 2.72 (d, J=11.0 Hz, 1H), 2.51 (d, J=7.8 Hz, 1H), 1.96 (s, 1H), 1.89-1.79 (m, 2H), 1.63 (s, 1H), 1.20 (q, J=4.8, 4.3 Hz, 2H), 1.17-1.02 (m, 2H).

### Example 59: 6-[6-(4-methylpiperazin-1-yl) pyridin-3-yl]-4-[(3S)-piperidin-3-ylamino]pyrido[3,2-d]pyrimidine-8-carboxamide

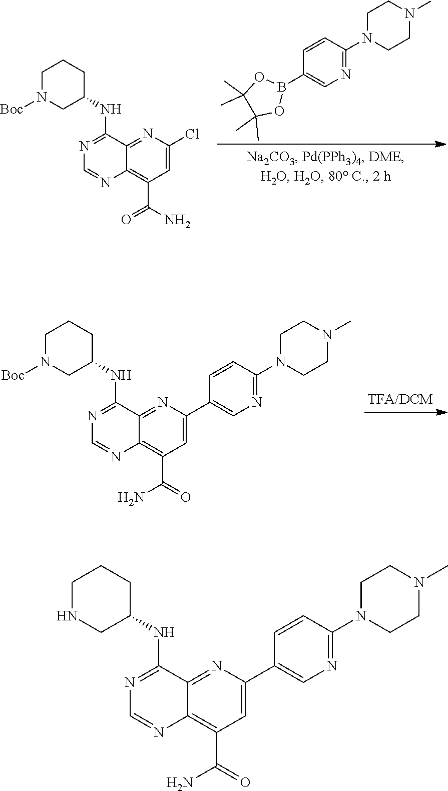

tert-butyl (3S)-3-([8-carbamoyl-6-[6-(4-methylpiperazin-1-yl) pyridin-3-yl]pyrido[3,2-d]pyrimidin-4-yl]amino) piperidine-1-carboxylate: To a solution of tert-butyl (3S)-3-([8-carbamoyl-6-chloropyrido[3,2-d]pyrimidin-4-yl]amino) piperidine-1-carboxylate (120 mg, 0.275 mmol) and 1-methyl-4-[5-(4,4,5,5-tetramethyl-1,3,2-dioxaborolan-2-yl) pyridin-2-yl]piperazine (120 mg, 0.376 mmol) in 1,4-dioxane (5.00 mL) and H₂O (1.00 mL) were added K₃PO₄ (168 mg, 0.752 mmol) and Pd(Amphos)Cl₂ (24 mg, 0.033 mmol,). The resulting mixture was stirred for 2 h at 100° C. under a nitrogen atmosphere. The residue was purified by silica gel column chromatography, eluted with CH₂Cl₂/MeCOH (9:1) to afford tert-butyl (3S)-3-([8-carbamoyl-6-[6-(4-methylpiperazin-1-yl) pyridin-3-yl]pyrido[3,2-d]pyrimidin-4-yl]amino) piperidine-1-carboxylate (130 mg, 78.93%) as a yellow solid.

6-[6-(4-methylpiperazin-1-yl) pyridin-3-yl]-4-[(3S)-piperidin-3-ylamino]pyrido [3,2-d]pyrimidine-8-carboxamide: To a stirred solution of tert-butyl (3S)-3-([8-carbamoyl-6-[6-(4-methylpiperazin-1-yl) pyridin-3-yl]pyrido[3,2-d]pyrimidin-4-yl]amino) piperidine-1-carboxylate (130 mg, 0.217 mmol) and HCl (g) in MeOH (5.00 mL, 10.510 mmol, 52.54 equiv, 12%) was stirred for 2 h at 25° C. under nitrogen atmosphere. The resulting mixture was concentrated under reduced pressure. The crude product (100 mg) was purified by Prep-HPLC with the following conditions (2#SHIMADZU (HPLC-01)): Column, XBridge Prep OBD C18 Column, 30*150 mm 5 um; mobile phase, Water (10 mmol/l NH₄HCO₃+0.05% NH₄OH) and ACN (20% Phase B up to 50% in 8 min); Detector, uv.254 nm product was obtained. This resulted in 6-[6-(4-methylpiperazin-1-yl) pyridin-3-yl]-4-[(3S)-piperidin-3-ylamino]pyrido[3,2-d]pyrimidine-8-carboxamide (50 mg, 51.14%) as a yellow solid.

HPLC: 96%, RT=2.914 min MS: m/z=448.1[M+H]^{+ 1}H NMR (400 MHz, DMSO-d6): 10.03 (d, J=3.6 Hz, 1H), 9.19 (d, J=2.4 Hz, 1H), 8.74 (s, 1H), 8.55-8.43 (m, 2H), 8.37 (d, J=8.7 Hz, 1H), 8.20 (d, J=3.7 Hz, 1H), 6.97 (d, J=9.1 Hz, 1H), 4.30 (d, J=10.6 Hz, 1H), 3.65 (t, J=5.1 Hz, 4H), 3.05 (d, J=9.7 Hz, 1H), 2.84 (d, J=12.3 Hz, 1H), 2.58 (d, J=10.4 Hz, 1H), 2.54 (s, 4H), 2.42 (t, J=5.1 Hz, 4H), 2.23 (s, 3H), 1.90 (s, 1H), 1.81 (d, J=10.4 Hz, 1H), 1.68 (s, 1H).

### Example 60: 6-(4-[[(3S)-3-fluoropyrrolidin-1-yl]methyl]phenyl)-4-[(3S)-piperidin-3-ylamino]pyrido[3,2-d]pyrimidine-8-carboxamide

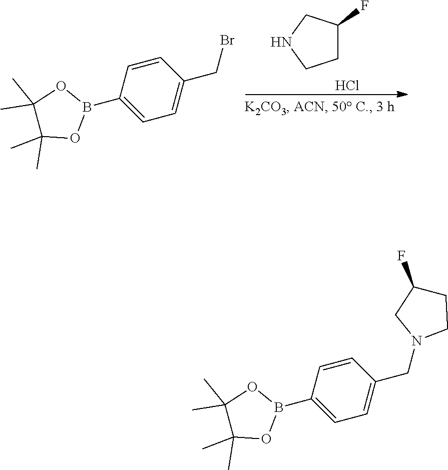 [SMILES: CC1(C)OB(c2ccc(CBr)cc2)OC1(C)C ; F[C@H]1CCNC1 ; Cl ; CC1(C)OB(c2ccc(CN3CC[C@H](F)C3)cc2)OC1(C)C]

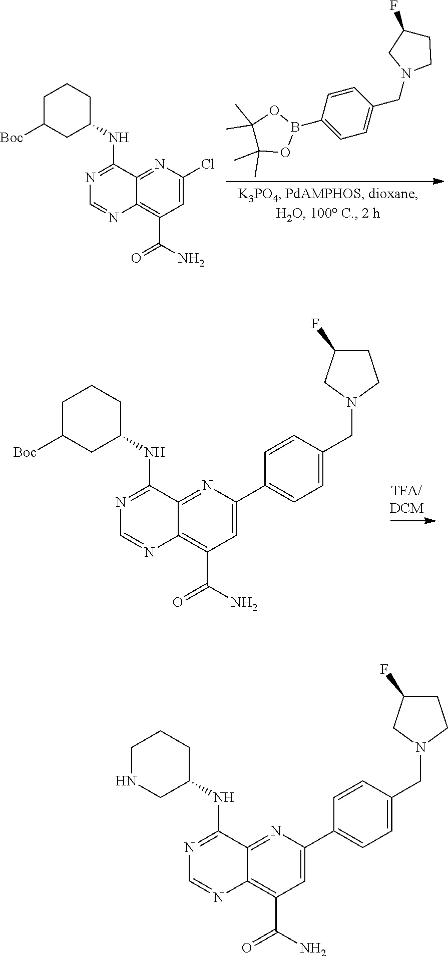

(3S)-3-fluoro-1-[[4-(4,4,5,5-tetramethyl-1,3,2-dioxaborolan-2-yl) phenyl]methyl]pyrrolidine: A solution of 2-[4-(bromomethyl) phenyl]-4,4,5,5-tetramethyl-1,3,2-dioxaborolane (1.00 g, 3.199 mmol), (3S)-3-fluoropyrrolidine hydrochloride (507 mg, 3.838 mmol) and K₂CO₃ (931 mg, 6.397 mmol) in ACN (20.00 mL) was stirred for 3 h at 50° C. The resulting mixture was concentrated under reduced pressure. This resulted in (3S)-3-fluoro-1-[[4-(4,4,5,5-tetramethyl-1,3,2-dioxaborolan-2-yl) phenyl]methyl]pyrrolidine (950 mg, 68.12%) as a white solid.

tert-butyl (3S)-3-[[8-carbamoyl-6-(4-[[(3S)-3-fluoropyrrolidin-1-yl]methyl]phenyl)pyrido[3,2-d]pyrimidin-4-yl]amino]piperidine-1-carboxylate: To a solution of tert-butyl (3S)-3-([8-carbamoyl-6-chloropyrido[3,2-d]pyrimidin-4-yl]amino)piperidine-1-carboxylate (150 mg, 0.343 mmol,) and (3S)-3-fluoro-1-[[4-(4,4,5,5-tetramethyl-1,3,2-dioxaborolan-2-yl)phenyl]methyl]pyrrolidine (290 mg, 0.665 mmol) in dioxane (10.00 mL,) and H₂O (2.00 mL) were added Pd(AMPHOS)Cl₂ (25 mg, 0.034 mmol) and K₃PO₄ (222 mg, 0.994 mmol). After stirring for 3 h at 100° C. under a nitrogen atmosphere. The residue was purified by silica gel column chromatography, eluted with CH₂Cl₂/MeOH (8:1) to afford tert-butyl (3S)-3-[[8-carbamoyl-6-(4-[[(3S)-3-fluoropyrrolidin-1-yl]methyl]phenyl) pyrido[3,2-d]pyrimidin-4-yl]amino]piperidine-1-carboxylate (190 mg, 73.55%) as a yellow oil.

6-(4-[[(3S)-3-fluoropyrrolidin-1-yl]methyl]phenyl)-4-[(3S)-piperidin-3-ylamino]pyrido[3,2-d]pyrimidine-8-carboxamide: Into a 50 mL round-bottom flask were added tert-butyl (3S)-3-[[8-carbamoyl-6-(4-[[(3S)-3-fluoropyrrolidin-1-yl]methyl]phenyl) pyrido[3,2-d]pyrimidin-4-yl]amino]piperidine-1-carboxylate (180 mg, 0.231 mmol), DCM (5.00 mL) and TFA (5.00 mL) at room temperature. The mixture was stirred for 2 h at room temperature under argon atmosphere. The resulting mixture was concentrated under reduced pressure. Desired product could be detected by LCMS. The residue was purified by Prep-HPLC to afford 6-(4-[[(3S)-3-fluoropyrrolidin-1-yl]methyl]phenyl)-4-[(3S)-piperidin-3-ylamino]pyrido[3,2-d]pyrimidine-8-carboxamide (44.8 mg, 41.51%) as a white solid.

HPLC: 96% RT=1.879 min MS: m/z=450.3 [M+H]¹H NMR (400 MHz, DMSO-d6):10.04 (s, 1H), 8.84 (s, 1H), 8.57 (s, 1H), 8.34 (dd, 3H), 8.23 (s, 1H), 7.51 (d, 2H), 5.12 (s, 1H), 4.35-4.26 (m, 1H), 3.71 (s, 2H), 3.06 (d, 1H), 2.85 (d, 1H), 2.84 (s, 1H), 2.77 (d, 1H), 2.73-2.64 (m, 1H), 2.69-2.52 (m, 2H), 2.36 (q, 2H), 2.25-2.08 (m, 1H), 1.96 (d, 1H), 1.89 (s, 1H), 1.81 (s, 1H), 1.68 (s, 1H), 1.52 (s, 1H).

### Example 61: 6-[4-(2-methoxy-2-methylpropoxy) phenyl]-4-[(3S)-piperidin-3-ylamino]pyrido[3,2-d]pyrimidine-8-carboxamide

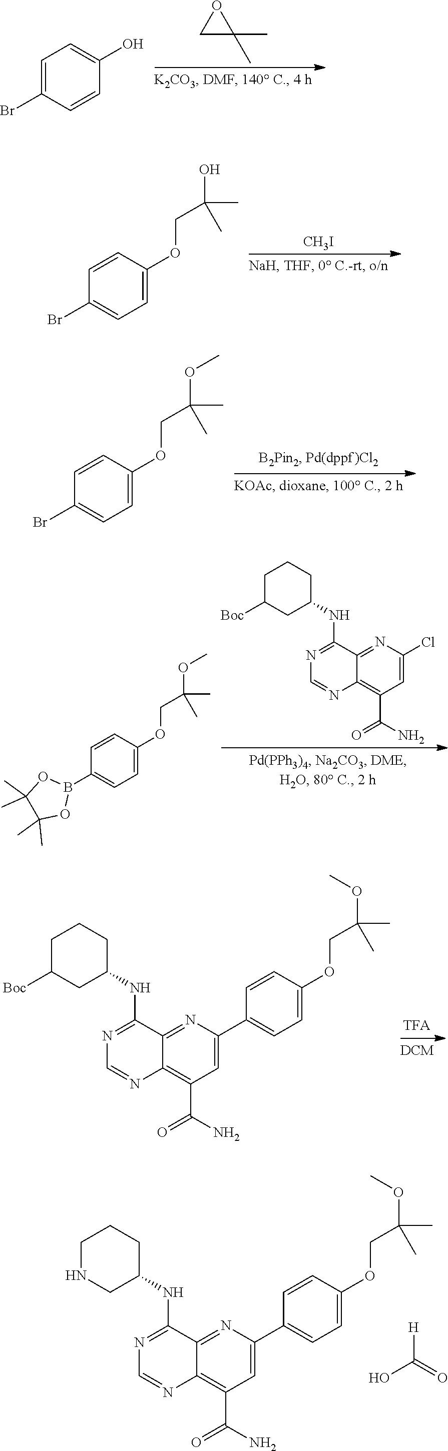

1-(4-bromophenoxy)-2-methylpropan-2-ol: To a stirred solution of 4-bromophenol (2.00 g, 10.982 mmol) and K₂CO₃ (4.79 g, 32.946 mmol) in DMF (20.00 mL) was added 2,2-dimethyloxirane (1.25 g, 16.469 mmol) in portions at room temperature under nitrogen atmosphere. The resulting mixture was stirred for 4 h at 140° C. under nitrogen atmosphere. The resulting mixture was added water (30 mL). The resulting mixture was extracted with EtOAc (3*30 mL). The combined organic layers were washed with brine (1*50 mL), dried over anhydrous Na₂SO₄. After filtration, the filtrate was concentrated under reduced pressure to afford 1-(4-bromophenoxy)-2-methylpropan-2-ol (2.71 g, 99.97%) as a brown solid.

1-bromo-4-(2-methoxy-2-methylpropoxy) benzene: To a stirred mixture of 1-(4-bromophenoxy)-2-methylpropan-2-ol (1.17 g, 4.692 mmol) in THF (20.00 mL, 234.517 mmol) was added NaH (0.35 g, 8.751 mmol) at 0° C. under nitrogen atmosphere. The resulting mixture was stirred for 10 min at 0° C. under nitrogen atmosphere. To the above mixture was added CH_{3I (}2.04 g, 13.654 mmol,) at 0° C. The resulting mixture was stirred for overnight at room temperature. The reaction was quenched with Water at room temperature. The resulting mixture was extracted with EtOAc (3*50 mL). The combined organic layers were washed with brine (1*100 mL), dried over anhydrous Na₂SO₄. After filtration, the filtrate was concentrated under reduced pressure. The residue was purified by reverse flash chromatography with the following conditions: column, C18 silica gel; mobile phase, ACN in water, 70% to 80% gradient in 10 min; detector, UV 254 nm. This resulted in 1-bromo-4-(2-methoxy-2-methylpropoxy) benzene (1.25 g, 99.92%) as a yellow oil.

2-[4-(2-methoxy-2-methylpropoxy) phenyl]-4,4,5,5-tetramethyl-1,3,2-dioxaborolane: To a stirred solution of 1-bromo-4-(2-methoxy-2-methylpropoxy) benzene (1.23 g, 4.614 mmol) and 4,4,5,5-tetramethyl-2-(tetramethyl-1,3,2-dioxaborolan-2-yl)-1,3,2-dioxaborolane (3.70 g, 13.841 mmol) in dioxane (10.00 mL) were added Pd(dppf)Cl₂ (0.36 g, 0.461 mmol) and KOAc (1.42 g, 13.702 mmol) at room temperature under nitrogen atmosphere. The resulting mixture was stirred for 2 h at 100° C. under nitrogen atmosphere. The reaction was quenched with Water at room temperature. The resulting mixture was extracted with EtOAc (3*20 mL). The combined organic layers were washed with brine (3*50 mL), dried over anhydrous Na₂SO₄. After filtration, the filtrate was concentrated under reduced pressure. The residue was purified by reverse flash chromatography with the following conditions: column, C18 silica gel; mobile phase, ACN in water, 65% to 75% gradient in 10 min; detector, UV 254 nm. This resulted in 2-[4-(2-methoxy-2-methylpropoxy) phenyl]-4,4,5,5-tetramethyl-1,3,2-dioxaborolane (1.46 g, 99.11%) as a white solid.

tert-butyl(3S)-3-([8-carbamoyl-6-[4-(2-methoxy-2-methylpropoxy) phenyl]pyrido[3,2-d]pyrimidin-4-yl]amino)piperidine-1-carboxylate: To a stirred solution tert-butyl (3S)-3-([8-carbamoyl-6-chloropyrido[3,2-d]pyrimidin-4-yl]amino) piperidine-1-carboxylate (180 mg, 0.311 mmol) and 2-[4-(2-methoxy-2-methylpropoxy) phenyl]-4,4,5,5-tetramethyl-1 3,2-dioxaborolane (272 mg, 0.852 mmol) in DME (2.40 mL) and H₂O (0.80 mL) were added Na₂CO₃ (188 mg, 1.685 mmol) and Pd(PPh₃)₄ (52 mg, 0.043 mmol) in portions at room temperature under nitrogen atmosphere. The resulting mixture was stirred for 2 h at 80° C. under nitrogen atmosphere. The residue was purified by silica gel column chromatography, eluted with 38% EtOAc in PE to afford tert-butyl (3S)-3-([8-carbamoyl-6-[4-(2-methoxy-2-methylpropoxy) phenyl]pyrido[3,2-d]pyrimidin-4-yl]amino) piperidine-1-carboxylate (196 mg, 99.80%) as a yellow solid.

6-[4-(2-methoxy-2-methylpropoxy) phenyl]-4-[(3S)-piperidin-3-ylamino]pyrido [3,2-d]pyrimidine-8-carboxamide; formic acid: To a stirred solution tert-butyl (3S)-3-([8-carbamoyl-6-[4-(2-methoxy-2-methylpropoxy) phenyl]pyrido[3,2-d]pyrimidin-4-yl]amino) piperidine-1-carboxylate (186 mg, 0.295 mmol) in DCM (2.40 mL) was added TFA (0.80 mL) dropwise at 0° C. under nitrogen atmosphere. The resulting mixture was stirred for 2 h at room temperature under nitrogen atmosphere. The crude product was purified by Prep-HPLC with the following conditions (2#SHIMADZU (HPLC-01)): Column, Xselect CSH OBD Column 30*150 mm 5 um, n; mobile phase, Water (0.1% FA) and ACN (30% Phase B up to 50% in 8 min); Detector, uv254 to afford 6-[4-(2-methoxy-2-methylpropoxy) phenyl]-4-[(3S)-piperidin-3-ylamino]pyrido[3,2-d]pyrimidine-8-carboxamide; formic acid (60.0 mg, 40.24%) as a yellow solid.

HPLC: 98%, RT=3.053 min MS: m/z=451.2 [M+H]^{+ 1}H NMR (400 MHz, DMSO-d6): 9.99 (d, J=3.7 Hz, 1H), 8.80 (d, J=2.0 Hz, 1H), 8.57 (d, J=1.2 Hz, 1H), 8.47 (d, J=7.8 Hz, 1H), 8.39-8.29 (m, 3H), 8.24-8.17 (m, 1H), 7.12 (dd, J=8.9, 3.0 Hz, 2H), 4.54 (s, 1H), 3.94 (d, J=1.7 Hz, 2H), 3.26 (s, 1H), 3.18 (s, 3H), 3.07 (s, 2H), 2.75 (s, 1H), 1.98 (s, 1H), 1.86 (d, J=11.6 Hz, 3H), 1.69 (s, 1H), 1.24 (s, 6H).

### Example 62: 6-[4-(2-hydroxy-2-methylpropoxy) phenyl]-4-[(3S)-piperidin-3-ylamino]pyrido[3,2-d]pyrimidine-8-carboxamide

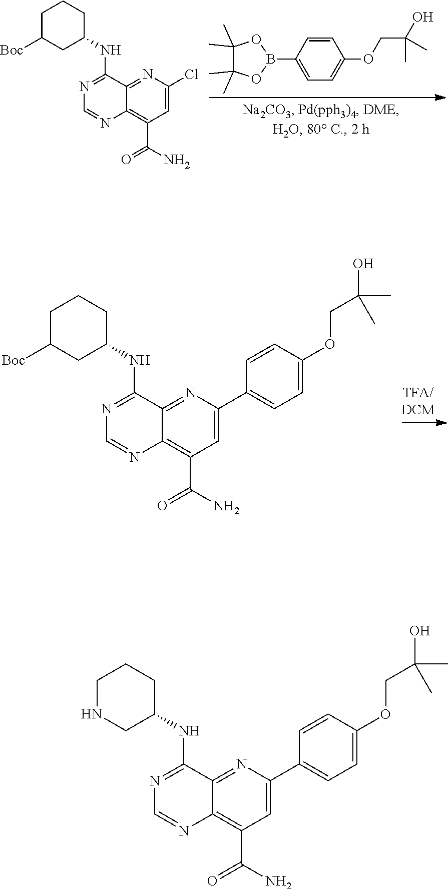

tert-butyl (3S)-3-([8-carbamoyl-6-[4-(2-hydroxy-2-methylpropoxy) phenyl]pyrido[3,2-d]pyrimidin-4-yl]amino) piperidine-1-carboxylate: To a stirred solution tert-butyl (3S)-3-([8-carbamoyl-6-chloropyrido[3,2-d]pyrimidin-4-yl]amino)piperidine-1-carboxylate (150 mg, 0.272 mmol) and 2-methyl-1-[4-(4,4,5,5-tetramethyl-1,3,2-dioxaborolan-2-yl)phenoxy]propan-2-ol (216 mg, 0.731 mmol) in DME (1.80 mL, 17.667 mmol) and H₂O (0.60 mL, 31.640 mmol) were added Na₂CO₃ (157 mg, 1.407 mmol) and Pd(PPh₃)₄ (43 mg, 0.035 mmol) in portions at room temperature under nitrogen atmosphere. The resulting mixture was stirred for 2 h at 80 degrees C. under nitrogen atmosphere. The residue was purified by silica gel column chromatography, eluted with 38% EtOAc in PE to afford tert-butyl (3S)-3-([8-carbamoyl-6-[4-(2-hydroxy-2-methylpropoxy) phenyl]pyrido[3,2-d]pyrimidin-4-yl]amino) piperidine-1-carboxylate (150 mg, 99.99%) as a yellow solid.

6-[4-(2-hydroxy-2-methylpropoxy) phenyl]-4-[(3S)-piperidin-3-ylamino]pyrido[3,2-d]pyrimidine-8-carboxamide: To a stirred solution tert-butyl (3S)-3-([8-carbamoyl-6-[4-(2-hydroxy-2-methylpropoxy) phenyl]pyrido[3,2-d]pyrimidin-4-yl]amino) piperidine-1-carboxylate (140 mg, 0.254 mmol) in DCM (1.80 mL, 26.898 mmol) was added TFA (0.60 mL, 7.674 mmol) dropwise at 0° C. under nitrogen atmosphere. The resulting mixture was stirred for 1 h at room temperature under nitrogen atmosphere. The resulting mixture was concentrated under reduced pressure. The crude product was purified by Prep-HPLC with the following conditions (2#SHIMADZU (HPLC-01)): Column, Xselect CSH OBD Column 30*150 mm 5 um, n; mobile phase, Water (0.1% FA) and ACN (35% Phase B up to 55% in 8 min); Detector, uv254 to afford 6-[4-(2-hydroxy-2-methylpropoxy) phenyl]-4-[(3S)-piperidin-3-ylamino]pyrido[3,2-d]pyrimidine-8-carboxamide (62.3 mg, 55.61%) as a yellow solid.

HPLC: 99% purity, RT=2.57 min. MS: m/z=437.10 [M+H]⁺. ¹H NMR (300 MHz, DMSO-d6) δ 10.02 (d, J=3.6 Hz, 1H), 8.82 (s, 1H), 8.58 (s, 1H), 8.47 (d, J=8.6 Hz, 1H), 8.35 (dd, J=9.8, 2.8 Hz, 2H), 8.23 (d, J=3.7 Hz, 1H), 7.17-7.07 (m, 2H), 4.51 (s, 1H), 3.83 (s, 2H), 3.25 (d, J=11.7 Hz, 1H), 3.12-2.92 (m, 2H), 2.79-2.66 (m, 1H), 2.51 (p, J=1.8 Hz, 2H), 1.98 (s, 1H), 1.85 (t, J=10.1 Hz, 2H), 1.67 (s, 1H), 1.25 (s, 6H).

### Example 63: 6-(4-[6-oxa-3-azabicyclo [3.1.1]heptan-3-ylmethyl]phenyl)-4-[(3S)-piperidin-3-ylamino]pyrido[3,2-d]pyrimidine-8-carboxamide

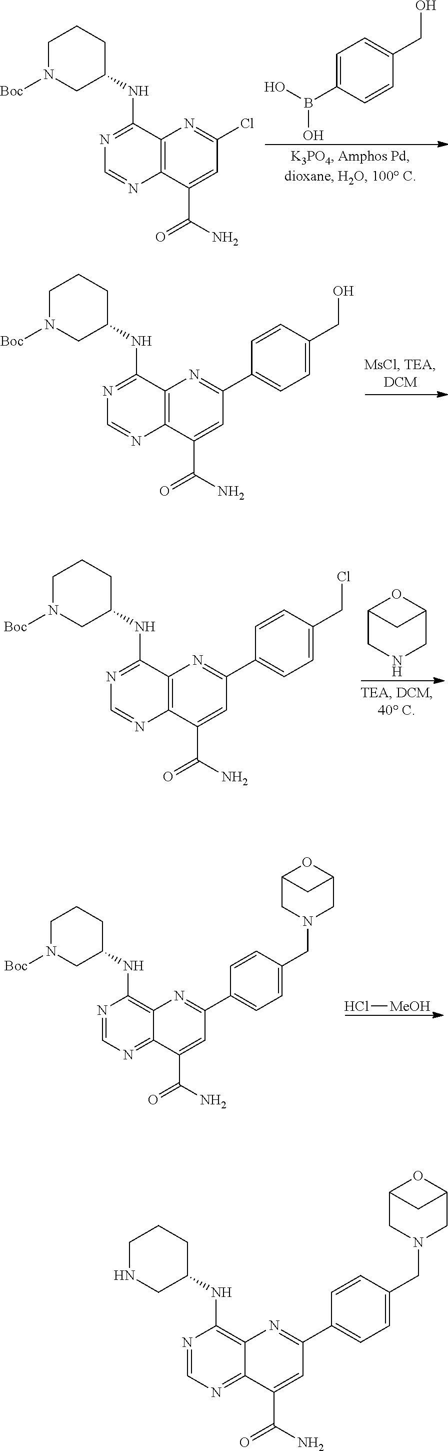

tert-butyl (3S)-3-([8-carbamoyl-6-[4-(hydroxymethyl) phenyl]pyrido[3,2-d]pyrimidin-4-yl]amino) piperidine-1-carboxylate: A solution of tert-butyl (3S)-3-([8-carbamoyl-6-chloropyrido[3,2-d]pyrimidin-4-yl]amino) piperidine-1-carboxylate (900.00 mg, 1.916 mmol) and 4-(hydroxymethyl)phenylboronic acid (502.52 mg, 3.142 mmol), Pd(Amphos)Cl₂ (157.00 mg, 0.211 mmol), K₃PO₄ (1.42 g, 6.360 mmol) in dioxane (5.00 mL) and H₂O (1.00 mL) was stirred for 2 h at 100° C. under nitrogen atmosphere. The resulting mixture was concentrated under vacuum. The residue was purified by silica gel column chromatography, eluted with CH₂Cl₂/MeOH (1:1) to afford tert-butyl (3S)-3-([8-carbamoyl-6-[4-(hydroxymethyl) phenyl]pyrido[3,2-d]pyrimidin-4-yl]amino) piperidine-1-carboxylate (1.1 g, 96.95%) as a yellow solid.

tert-butyl (3S)-3-([8-carbamoyl-6-[4-(chloromethyl) phenyl]pyrido[3,2-d]pyrimidin-4-yl]amino) piperidine-1-carboxylate: To a stirred solution of tert-butyl (3S)-3-([8-carbamoyl-6-[4-(hydroxymethyl) phenyl]pyrido[3,2-d]pyrimidin-4-yl]amino) piperidine-1-carboxylate (500.00 mg, 0.993 mmol) and TEA (317.17 mg, 2.978 mmol) in DCM (25.00 mL) was added MsCl (239.37 mg, 1.985 mmol) in portions at 0° C. The resulting mixture was stirred for 16 h at 25° C. The resulting mixture was concentrated under vacuum. The residue was purified by silica gel column chromatography, eluted with PE/EtOAc (3:7) to afford tert-butyl (3S)-3-([8-carbamoyl-6-[4-(chloromethyl) phenyl]pyrido[3,2-d]pyrimidin-4-yl]amino) piperidine-1-carboxylate (290 mg, 57.14%) as a yellow solid.

tert-butyl (3S)-3-((6-(4-((6-oxa-3-azabicyclo [3.1.1]heptan-3-yl) methyl) phenyl)-8-carbamoylpyrido[3,2-d]pyrimidin-4-yl) amino) piperidine-1-carboxylate: To a solution of tert-butyl (3S)-3-([8-carbamoyl-6-[4-(chloromethyl) phenyl]pyrido[3,2-d]pyrimidin-4-yl]amino) piperidine-1-carboxylate (200.00 mg, 0.185 mmol), 6-oxa-3-azabicyclo[3.1.1]heptane (38.55 mg, 0.369 mmol), K₂CO₃ (80.61 mg, 0.554 mmol) in CAN (1.00 mL, 24.360 mmol) was stirred for 16 h at 80° C. under nitrogen atmosphere. The precipitated solids were collected by filtration. The resulting mixture was concentrated under vacuum to afford tert-butyl (3S)-3-((6-(4-((6-oxa-3-azabicyclo [3.1.1] heptan-3-yl) methyl) phenyl)-8-carbamoylpyrido[3,2-d]pyrimidin-4-yl) amino) piperidine-1-carboxylate (70.00 mg; crude product) as a yellow solid.

6-(4-[6-oxa-3-azabicyclo [3.1.1]heptan-3-ylmethyl]phenyl)-4-[(3S)-piperidin-3-ylamino]pyrido[3,2-d]pyrimidine-8-carboxamide: To a solution of tert-butyl (3S)-3-[[8-carbamoyl-6-(4-[6-oxa-3-azabicyclo[3.1.1]heptan-3-ylmethyl]phenyl) pyrido[3,2-d]pyrimidin-4-yl]amino]piperidine-1-carboxylate (60.00 mg, 0.071 mmol) and TFA (0.30 mL, 2.499 mmol) in DCM (1.00 mL, 15.730 mmol) was stirred for 2 h at 25 degrees C. under nitrogen atmosphere. The resulting mixture was concentrated under vacuum. The reaction was quenched with Water at 0° C. The aqueous layer was extracted with EtOAc (3×1 L). The crude product (30 mg) was purified by Prep-HPLC with the following conditions (2#SHIMADZU (HPLC-01)): Column, Xselect Peptide CSH 19*150 mm Sum; mobile phase, water (10 mmol/l NH₄HCO₃) and ACN (26% Phase B up to 42% in 8 min); Detector, UV. product was obtained. This resulted in 6-(4-[6-oxa-3-azabicyclo [3.1.1]heptan-3-ylmethyl]phenyl)-4-[(3S)-piperidin-3-ylamino]pyrido[3,2-d]pyrimidine-8-carboxamide (10 mg, 29.77%) as a yellow solid.

HPLC: 97% purity, RT=2.97 min. MS: m/z=460.00 [M+H]⁺. ¹H NMR (300 MHz, DMSO-d6) δ 10.05 (d, J=3.6 Hz, 1H), 8.83 (s, 1H), 8.58 (s, 1H), 8.40-8.21 (m, 4H), 7.58 (d, J=8.1 Hz, 2H), 4.49-4.22 (m, 1H), 3.84 (s, 2H), 3.74-3.60 (m, 2H), 3.19-3.04 (m, 2H), 2.87-2.68 (m, 2H), 2.52 (d, J=1.9 Hz, 2H), 1.98-1.70 (m, 2H), 1.51 (d, J=11.6 Hz, 1H), 1.17-0.66 (m, 6H).

### Example 64: 6-(4-[3,7-dioxa-9-azabicyclo [3.3.1]nonan-9-ylmethyl]phenyl)-4-[(3S)-piperidin-3-ylamino]pyrido[3,2-d]pyrimidine-8-carboxamide

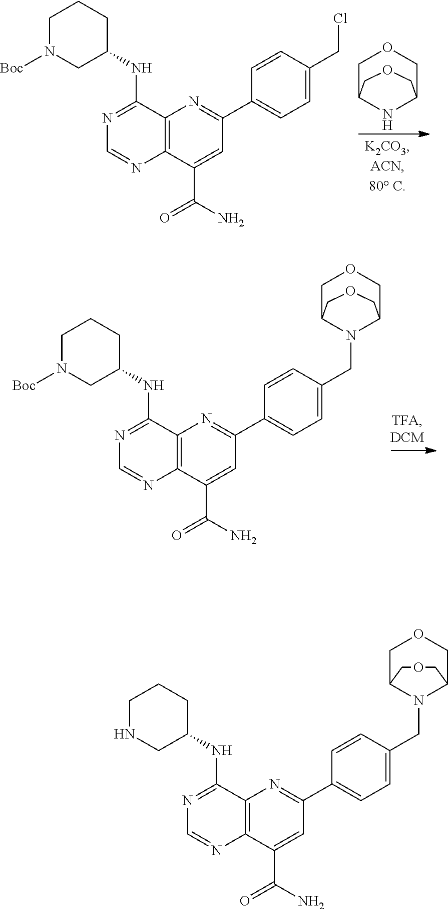

tert-butyl (3S)-3-[[8-carbamoyl-6-(4-[3,7-dioxa-9-azabicyclo [3.3.1]nonan-9-ylmethyl]phenyl)pyrido[3,2-d]pyrimidin-4-yl]amino]piperidine-1-carboxylate: To a mixture of tert-butyl (3S)-3-([8-carbamoyl-6-[4-(chloromethyl)phenyl]pyrido[3,2-d]pyrimidin-4-yl]amino)piperidine-1-carboxylate (100.00 mg, 0.196 mmol), (1s,5s)-3,7-dioxa-9-azabicyclo[3.3.1]nonane (37.926 mg, 0.196 mmol) and K₂CO₃ (85.36 mg, 0.587 mmol) in ACN (2.00 mL) was stirred for overnight at 80° C. under nitrogen atmosphere. The reaction was quenched with water at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. This resulted in tert-butyl (3S)-3-[[8-carbamoyl-6-(4-[3,7-dioxa-9-azabicyclo [3.3.1]nonan-9-ylmethyl]phenyl)pyrido[3,2-d]pyrimidin-4-yl]amino]piperidine-1-carboxylate (120 mg, 88.65%) as a light yellow solid.

6-(4-[3,7-dioxa-9-azabicyclo [3.3.1]nonan-9-ylmethyl]phenyl)-4-[(3S)-piperidin-3-ylamino]pyrido[3,2-d]pyrimidine-8-carboxamide: A mixture of tert-butyl (3S)-3-[[8-carbamoyl-6-(4-[3,7-dioxa-9-azabicyclo[3.3.1]nonan-9-ylmethyl]phenyl) pyrido[3,2-d]pyrimidin-4-yl]amino]piperidine-1-carboxylate (110.00 mg, 0.159 mmol) in HCl (g) in MeCOH (5.50 mL, 21.722 mmol) was stirred for 1 h at room temperature under nitrogen atmosphere. The resulting mixture was concentrated under vacuum. The crude product (80 mg) was purified by Prep-HPLC with the following conditions (2#SHIMADZU (HPLC-01)): Column, XBridge Shield RP18 OBD Column, 30*150 mm, 5 um; mobile phase, Water (10 mmol/l NH₄HCO₃+0.05% NH₄OH) and ACN (5% Phase B up to 30% in 8 min); Detector, uv. This resulted in 6-(4-[3,7-dioxa-9-azabicyclo [3.3.1]nonan-9-ylmethyl]phenyl)-4-[(3S)-piperidin-3-ylamino]pyrido[3,2-d]pyrimidine-8-carboxamide (18.6 mg, 23.79%) as a light yellow solid.

HPLC: 99% purity, RT=2.69 min. MS: m/z=490.40 [M+H]⁺. ¹H NMR (300 MHz, DMSO-d6) δ 10.05 (d, J=3.6 Hz, 1H), 8.82 (s, 1H), 8.56 (s, 1H), 8.33 (dd, J=16.0, 8.3 Hz, 3H), 8.23 (d, J=3.6 Hz, 1H), 7.58 (d, J=8.1 Hz, 2H), 4.28 (dd, J=8.8, 4.4 Hz, 1H), 4.12 (s, 2H), 4.02 (dd, J=10.6, 2.5 Hz, 4H), 3.73 (d, J=10.9 Hz, 4H), 3.04 (d, J=10.3 Hz, 1H), 2.79 (s, 1H), 2.72 (dd, J=11.6, 8.6 Hz, 1H), 2.58 (d, J=10.8 Hz, 1H), 2.43 (s, 2H), 1.88 (s, 2H), 1.66 (s, 1H), 1.49 (d, J=11.2 Hz, 1H).

### Example 65: 6-(4-[8-oxa-3-azabicyclo [3.2.1]octan-3-ylmethyl]phenyl)-4-[(3S)-piperidin-3-ylamino]pyrido[3,2-d]pyrimidine-8-carboxamide

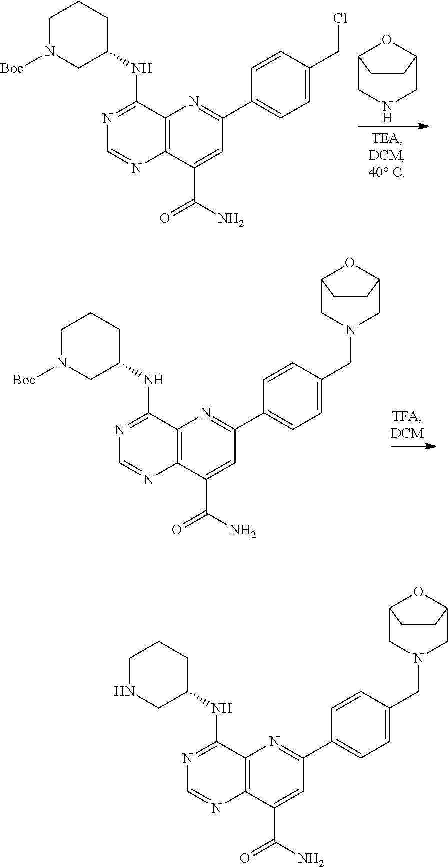

tert-butyl (3S)-3-[[8-carbamoyl-6-(4-[8-oxa-3-azabicyclo [3.2.1]octan-3-ylmethyl]phenyl)pyrido[3,2-d]pyrimidin-4-yl]amino]piperidine-1-carboxylate: To A solution of tert-butyl (3S)-3-([8-carbamoyl-6-[4-(hydroxymethyl)phenyl]pyrido[3,2-d]pyrimidin-4-yl]amino)piperidine-1-carboxylate (85.00 mg, 0.128 mmol), 8-oxa-3-azabicyclo[3.2.1]octane (49.95 mg, 0.419 mmol) and TEA (51.7 mg, 0.512 mmol) in DCM (3.00 mL) was stirred for 4 h at 40° C. The resulting mixture was concentrated under vacuum. to afford tert-butyl (3S)-3-[[8-carbamoyl-6-(4-[8-oxa-3-azabicyclo[3.2.1]octan-3-ylmethyl]phenyl) pyrido[3,2-d]pyrimidin-4-yl]amino]piperidine-1-carboxylate (60 mg, 62.55%) as a yellow solid.

6-(4-[8-oxa-3-azabicyclo [3.2.1]octan-3-ylmethyl]phenyl)-4-[(3S)-piperidin-3-ylamino]pyrido[3,2-d]pyrimidine-8-carboxamide: To a solution of tert-butyl (3S)-3-[[8-carbamoyl-6-(4-[8-oxa-3-azabicyclo[3.2.1]octan-3-ylmethyl]phenyl)pyrido[3,2-d]pyrimidin-4-yl]amino]piperidine-1-carboxylate (60.00 mg, 0.080 mmol) in DCM (3.00 mL) and TFA (1.00 mL) was stirred for 1 h at room temperature. The resulting mixture was concentrated under reduced pressure. The crude product was purified by Prep-HPLC with the following conditions (2#SHIMADZU (HPLC-01)): Column, Xselec CSH OBD Column 30*150 mm Sum; mobile phase, water (10 mmol/l NH₄HCO₃+0.05% NH₄OH) and ACN (36% Phase B up to 47% in 7 min); Detector, uv. product was obtained. This resulted in 6-(4-[8-oxa-3-azabicyclo [3.2.1]octan-3-ylmethyl]phenyl)-4-[(3S)-piperidin-3-ylamino]pyrido[3,2-d]pyrimidine-8-carboxamide (11.0 mg, 28.03%) as a light yellow solid.

HPLC: 97% purity, RT=2.84 min. MS: m/z=474.30. [M+H]⁺. ¹H NMR (300 MHz, DMSO-d6, ppm) 10.04 (brs, 1H), 8.81 (s, 1H), 8.55 (s, 1H), 8.39-8.15 (m, 4H), 7.47 (d, J=8.1 Hz, 2H), 4.20 (s, 3H), 3.51 (s, 2H), 3.03 (d, J=10.7 Hz, 1H), 2.81-2.53 (m, 4H), 2.22 (d, J=10.7 Hz, 3H), 1.94-1.67 (m, 7H), 1.48 (s, 1H)

### Example 66: 6-(4-[3-oxa-8-azabicyclo [3.2.1]octan-8-ylmethyl]phenyl)-4-[(3S)-piperidin-3-ylamino]pyrido[3,2-d]pyrimidine-8-carboxamide

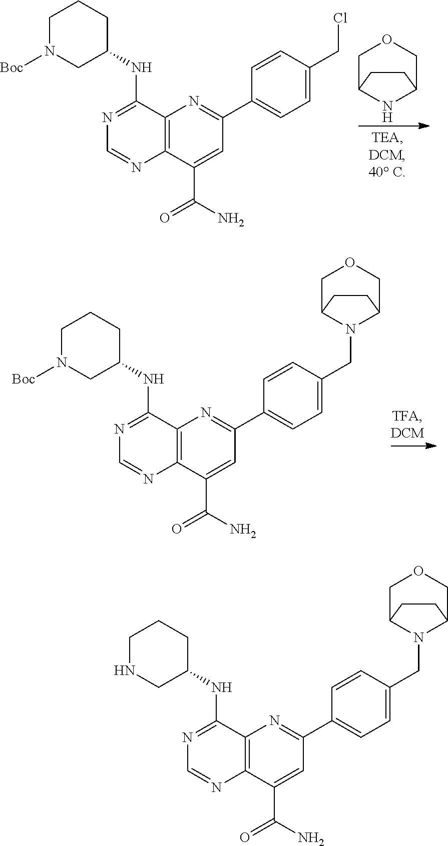

tert-butyl (3S)-3-[[8-carbamoyl-6-(4-[3-oxa-8-azabicyclo [3.2.1]octan-8-ylmethyl]phenyl)pyrido[3,2-d]pyrimidin-4-yl]amino]piperidine-1-carboxylate: To A solution of tert-butyl (3S)-3-([8-carbamoyl-6-[4-(hydroxymethyl)phenyl]pyrido[3,2-d]pyrimidin-4-yl]amino)piperidine-1-carboxylate (85.00 mg, 0.128 mmol), 3-oxa-8-azabicyclo[3.2.1]octane (49.95 mg, 0.419 mmol) and TEA (51.7 mg, 0.512 mmol) in DCM (3.00 mL) was stirred for 4 h at 40° C. The resulting mixture was concentrated under vacuum to afford tert-butyl (3S)-3-[[8-carbamoyl-6-(4-[3-oxa-8-azabicyclo [3.2.1]octan-8-ylmethyl]phenyl)pyrido[3,2-d]pyrimidin-4-yl]amino]piperidine-1-carboxylate (60 mg, 23.00%) as a yellow solid.

6-(4-[3-oxa-8-azabicyclo [3.2.1]octan-8-ylmethyl]phenyl)-4-[(3S)-piperidin-3-ylamino]pyrido[3,2-d]pyrimidine-8-carboxamide: To A solution of tert-butyl (3S)-3-[[8-carbamoyl-6-(4-[3-oxa-8-azabicyclo[3.2.1]octan-8-ylmethyl]phenyl)pyrido[3,2-d]pyrimidin-4-yl]amino]piperidine-1-carboxylate (65.00 mg, 0.032 mmol) and TFA (1.00 mL, 13.463 mmol) in DCM (3.00 mL, 35.322 mmol) was stirred for 1 h at 25° C. The resulting mixture was concentrated under vacuum. The crude product was purified by Prep-HPLC with the following conditions (2#SHIMADZU (HPLC-01)): Column, XBridge Shield RP18 OBD Column, 19*250 mm, 10 um; mobile phase, water (10 mmol/l NH₄HCO₃+0.05% NH₄OH) and ACN (39% Phase B up to 43% in 10 min); Detector, uv. product was obtained. This resulted in 6-(4-[3-oxa-8-azabicyclo octan-8-ylmethyl]phenyl)-4-[(3S)-piperidin-3-ylamino]pyrido[3,2-d]pyrimidine-8-carboxamide (8.9 mg, 57.17%) as a yellow solid.

HPLC: 97% purity, RT=2.19 min. MS: m/z=474.40 [M+H]⁺. ¹H NMR (300 MHz, DMSO-d6, ppm) 10.05 (brs, 1H), 8.81 (s, 1H), 8.55 (s, 1H), 8.38-8.18 (m, 4H), 7.56 (d, J=8.0 Hz, 2H), 4.26 (s, 1H), 3.61-3.48 (m, 4H), 3.41 (d, J=10.0 Hz, 2H), 3.02 (d, J=16.5 Hz, 3H), 2.75 (s, 2H), 2.57 (d, J=10.4 Hz, 1H), 1.97-1.67 (m, 7H), 1.48 (s, 1H).

### Example 67: 6-(4-[3-oxa-6-azabicyclo [3.1.1]heptan-6-ylmethyl]phenyl)-4-[(3S)-piperidin-3-ylamino]pyrido[3,2-d]pyrimidine-8-carboxamide

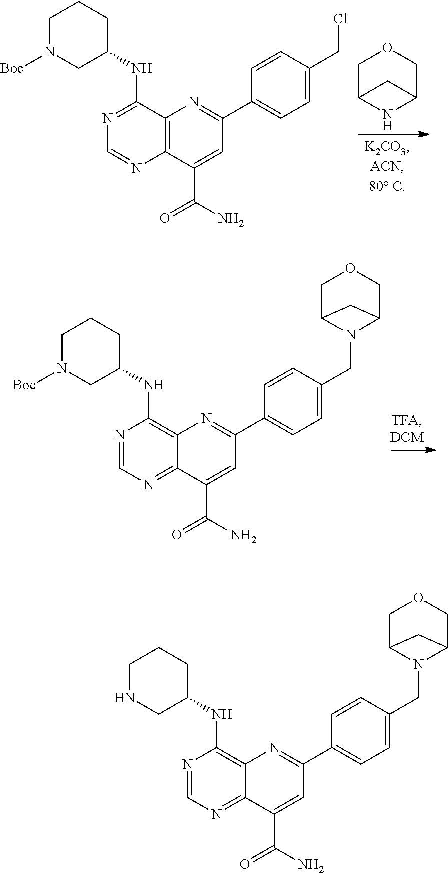

tert-butyl (3S)-3-[[8-carbamoyl-6-(4-[3-oxa-6-azabicyclo [3.1.1]heptan-6-ylmethyl]phenyl)pyrido[3,2-d]pyrimidin-4-yl]amino]piperidine-1-carboxylate: A solution of tert-butyl (3S)-3-([8-carbamoyl-6-[4-(chloromethyl)phenyl]pyrido[3,2-d]pyrimidin-4-yl]amino)piperidine-1-carboxylate (200.00 mg, 0.185 mmol) and 3-oxa-6-azabicyclo[3.1.1]heptane (218.00 mg, 2.089 mmol), K₂CO₃ (222.00 mg, 1.526 mmol) in CAN (2.00 mL) was stirred for 16 h at 80° C. under nitrogen atmosphere. The precipitated solids were collected by filtration. The resulting mixture was concentrated under vacuum to afford tert-butyl (3S)-3-[[8-carbamoyl-6-(4-[3-oxa-6-azabicyclo [3.1.1]heptan-6-ylmethyl]phenyl)pyrido[3,2-d]pyrimidin-4-yl]amino]piperidine-1-carboxylate (70 mg, 45.77%) as a yellow solid.

6-(4-[3-oxa-6-azabicyclo [3.1.1]heptan-6-ylmethyl]phenyl)-4-[(3S)-piperidin-3-ylamino]pyrido[3,2-d]pyrimidine-8-carboxamide: A solution of tert-butyl (3S)-3-[[8-carbamoyl-6-(4-[3-oxa-6-azabicyclo[3.1.1]heptan-6-ylmethyl]phenyl)pyrido[3,2-d]pyrimidin-4-yl]amino]piperidine-1-carboxylate (65.00 mg, 0.079 mmol) and TFA (0.30 mL) in DCM (1.00 mL) was stirred for 2 h at 25° C. under nitrogen atmosphere. The reaction was quenched with Water/Ice at 0° C. The aqueous layer was extracted with EtOAc (3×10 mL). The crude product (25 mg) was purified by Prep-HPLC with the following conditions (2#SHIMADZU (HPLC-01)): Column, XBridge Prep OBD C18 Column, 30*150 mm Sum; mobile phase, Water (10 mmol/I NH₄HCO₃+0.05% NH₄OH) and ACN (15% Phase B up to 40% in 8 min); Detector, uv. The resulting mixture was concentrated under vacuum. This resulted in 6-(4-[3-oxa-6-azabicyclo [3.1.1]heptan-6-ylmethyl]phenyl)-4-[(3S)-piperidin-3-ylamino]pyrido[3,2-d]pyrimidine-8-carboxamide (18 mg, 46.90%) as a yellow solid.

HPLC: 94% purity, RT=3.276 min. MS: m/z=460.20 [M+H]⁺. ¹H NMR (400 MHz, DMSO-d6) δ 10.04 (d, J=3.7 Hz, 1H), 8.82 (s, 1H), 8.56 (s, 1H), 8.35 (d, J=8.6 Hz, 2H), 8.28 (d, J=8.0 Hz, 1H), 8.21 (d, J=3.6 Hz, 2H), 7.57 (d, J=8.1 Hz, 1H), 4.34-4.24 (m, 2H), 4.19 (d, J=10.7 Hz, 1H), 3.88 (s, 2H), 3.65 (d, J=10.7 Hz, 2H), 3.44 (d, J=6.0 Hz, 2H), 3.31 (s, 2H), 3.07-2.93 (m, 1H), 2.84-2.70 (m, 2H), 2.56 (d, J=8.6 Hz, 2H), 1.97-1.87 (m, 2H), 1.80 (d, J=22.6 Hz, 1H), 1.49 (d, J=11.6 Hz, 1H).

### Example 68: 6-(4-[[(2R,6R)-2,6-dimethylmorpholin-4-yl]methyl]phenyl)-4-[(3S)-piperidin-3-ylamino]pyrido[3,2-d]pyrimidine-8-carboxamide

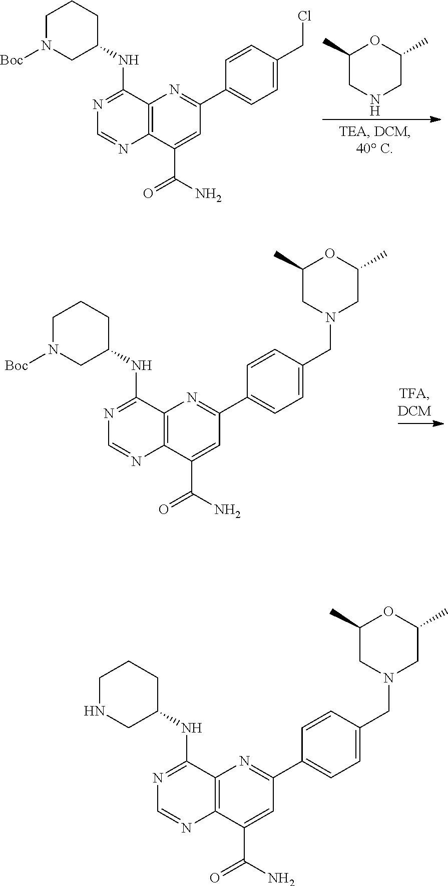

tert-butyl (3S)-3-[[8-carbamoyl-6-(4-[[(2R,6R)-2,6-dimethylmorpholin-4-yl]methyl]phenyl) pyrido[3,2-d]pyrimidin-4-yl]amino]piperidine-1-carboxylate: To a stirred solution of tert-butyl (3S)-3-([8-carbamoyl-6-[4-(chloromethyl)phenyl]pyrido[3,2-d]pyrimidin-4-yl]amino)piperidine-1-carboxylate (100.00 mg, 0.145 mmol) and (2R,6R)-2,6-dimethylmorpholine (40.00 mg, 0.330 mmol) in DCM (5.00 mL) was added TEA (61 mg, 0.6 mmol) in portions at room temperature. The resulting mixture was stirred for 4 h at 40° C. The resulting mixture was concentrated under vacuum to afford tert-butyl (3S)-3-[[8-carbamoyl-6-(4-[[(2R,6R)-2,6-dimethylmorpholin-4-yl]methyl]phenyl) pyrido[3,2-d]pyrimidin-4-yl]amino]piperidine-1-carboxylate (85 mg, 63.32%) as a yellow solid.

6-(4-[[(2R,6R)-2,6-dimethylmorpholin-4-yl]methyl]phenyl)-4-[(3S)-piperidin-3-ylamino]pyrido[3,2-d]pyrimidine-8-carboxamide: A solution of tert-butyl (3S)-3-[[8-carbamoyl-6-(4-[[(2R,6R)-2,6-dimethylmorpholin-4-yl]methyl]phenyl)pyrido[3,2-d]pyrimidin-4-yl]amino]piperidine-1-carboxylate (45.00 mg, 0.049 mmol) in TFA (1.00 mL) and DCM (3.00 mL) was stirred for 2 h at room temperature. The resulting mixture was concentrated under vacuum. The crude product was purified by Prep-HPLC with the following conditions (2#SHIMADZU (HPLC-01)): Column, XBridge Prep OBD C18 Column, 30*150 mm 5 um; mobile phase, Water (10 mmol/l NH₄HCO₃+0.05% NH₄OH) and ACN (35% Phase B up to 65% in 8 min); Detector, uv. 6-(4-[[(2R,6R)-2,6-dimethylmorpholin-4-yl]methyl]phenyl)-4-[(3S)-piperidin-3-ylamino]pyrido[3,2-d]pyrimidine-8-carboxamide (4.8 mg, 16.84%) as a light-yellow solid.

HPLC: 93% purity, RT=3.017 min. MS: m/z=476.2 [M+H]⁺. ¹H NMR (300 MHz, DMSO-d6, ppm) δ 9.89-9.39 (m, 1H), 8.98 (s, 1H), 8.76 (s, 1H), 8.58 (d, J=7.9 Hz, 2H), 7.88 (d, J=7.9 Hz, 2H), 4.84 (s, 1H), 4.52-4.30 (m, 2H), 4.20 (s, 2H), 3.42 (d, J=10.5 Hz, 1H), 3.30 (t, J=11.7 Hz, 4H), 3.01-2.78 (m, 3H), 2.07-1.83 (m, 4H), 1.42-1.30 (m, 3H), 1.08 (s, 3H).

### Example 69: 6-(4-[[(2R)-2-methylmorpholin-4-yl]methyl]phenyl)-4-[(3S)-piperidin-3-ylamino]pyrido[3,2-d]pyrimidine-8-carboxamide dihydrochloride

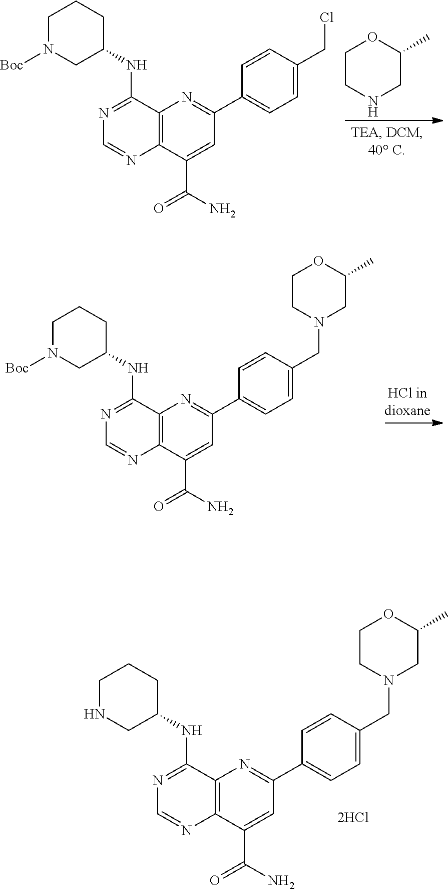

tert-butyl (3S)-3-[[8-carbamoyl-6-(4-[[(2R)-2-methylmorpholin-4-yl]methyl]phenyl)pyrido [3,2-d]pyrimidin-4-yl]amino]piperidine-1-carboxylate: A solution of tert-butyl (3S)-3-([8-carbamoyl-6-[4-(chloromethyl)phenyl]pyrido[3,2-d]pyrimidin-4-yl]amino)piperidine-1-carboxylate (150.00 mg, 0.203 mmol) and (2R)-2-methylmorpholine (43.25 mg, 0.406 mmol), TEA (86.54 mg, 0.812 mmol) in DCM (2 mL) was stirred for 3 h at 40° C. under nitrogen atmosphere. The resulting mixture was concentrated under vacuum. The crude product was used in the next step directly without further purification. This resulted in tert-butyl (3S)-3-[[8-carbamoyl-6-(4-[[(2R)-2-methylmorpholin-4-yl]methyl]phenyl) pyrido[3,2-d]pyrimidin-4-yl]amino]piperidine-1-carboxylate (100 mg, 87.65%) as a yellow solid.

6-(4-[[(2R)-2-methylmorpholin-4-yl]methyl]phenyl)-4-[(3S)-piperidin-3-ylamino]pyrido[3,2-d]pyrimidine-8-carboxamide dihydrochloride: A solution of tert-butyl (3S)-3-[[8-carbamoyl-6-(4-[[(2R)-2-methylmorpholin-4-yl]methyl]phenyl)pyrido[3,2-d]pyrimidin-4-yl]amino]piperidine-1-carboxylate (55.00 mg, 0.068 mmol) in HCl (gas) in 1,4-dioxane (1.00 mL) was stirred for 2 h at room temperature. The resulting mixture was concentrated under vacuum. The crude product (20 mg) was purified by Prep-HPLC with the following conditions (2#SHIMADZU (HPLC-01)): Column, XBridge Shield RP18 OBD Column, 30*150 mm, 5 um; mobile phase, Water (0.05% HCl) and ACN (3% Phase B up to 20% in 8 min); Detector, uv. product was obtained. This resulted in 6-(4-[[(2R)-2-methylmorpholin-4-yl]methyl]phenyl)-4-[(3S)-piperidin-3-ylamino]pyrido [3,2-d]pyrimidine-8-carboxamide dihydrochloride (15 mg, 23.70%) as a yellow solid.

HPLC: 90% purity, RT=3.085 min. MS: m/z=462.15 [M+H]⁺. ¹H NMR (300 MHz, DMSO-d6) δ 8.98 (s, 1H), 8.73 (s, 1H), 8.66-8.53 (m, 2H), 7.84 (d, J=8.2 Hz, 2H), 4.79 (s, 1H), 4.51-4.37 (m, 2H), 3.96 (s, 3H), 3.48-3.37 (m, 1H), 3.35-3.17 (m, 4H), 3.08 (t, J=11.3 Hz, 1H), 2.91-2.74 (m, 2H), 2.09-1.73 (m, 2H), 1.34 (t, J=7.0 Hz, 4H), 1.11 (d, J=6.3 Hz, 3H).

### Example 70: 6-[4-[(2,2-difluoromorpholin-4-yl) methyl]phenyl]-4-[(3S)-piperidin-3-ylamino]pyrido[3,2-d]pyrimidine-8-carboxamide

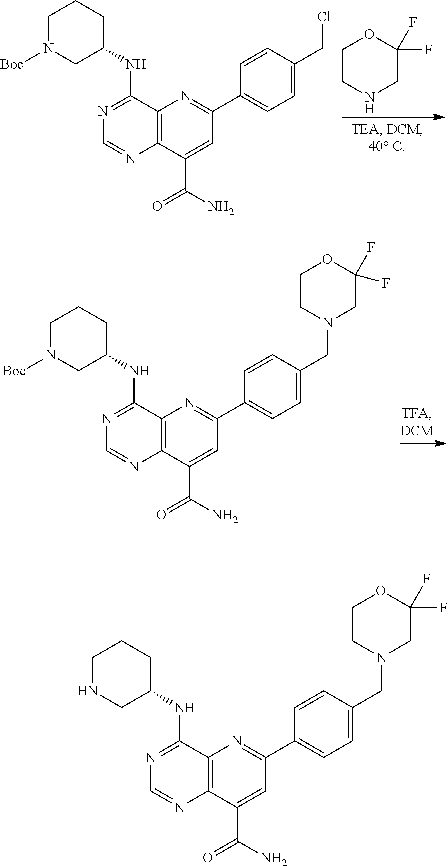

tert-butyl (3S)-3-[(8-carbamoyl-6-[4-[(2,2-difluoromorpholin-4-yl) methyl]phenyl]pyrido[3,2-d]pyrimidin-4-yl) amino]piperidine-1-carboxylate: A solution of tert-butyl (3S)-3-([8-carbamoyl-6-[4-(chloromethyl)phenyl]pyrido[3,2-d]pyrimidin-4-yl]amino)piperidine-1-carboxylate (85.00 mg, 0.123 mmol), TEA (54 mg, 0.5 mmol) and 2,2-difluoromorpholine (43.00 mg, 0.332 mmol) in DCM (5.00 mL) was stirred for 4 h at 40° C. The resulting mixture was concentrated under vacuum. to afford tert-butyl (3S)-3-[(8-carbamoyl-6-[4-[(2,2-difluoromorpholin-4-yl) methyl]phenyl]pyrido[3,2-d]pyrimidin-4-yl) amino]piperidine-1-carboxylate (55 mg, 52.32%) as a yellow solid.

6-[4-[(2,2-difluoromorpholin-4-yl) methyl]phenyl]-4-[(3S)-piperidin-3-ylamino]pyrido[3,2-d]pyrimidine-8-carboxamide: A solution of tert-butyl (3S)-3-[(8-carbamoyl-6-[4-[(2,2-difluoromorpholin-4-yl) methyl]phenyl]pyrido[3,2-d]pyrimidin-4-yl) amino]piperidine-1-carboxylate (50.00 mg, 0.061 mmol) and TFA (1.00 mL) in DCM (3.00 mL) was stirred for 1 h at room temperature. The resulting mixture was concentrated under vacuum. The crude product was purified by Prep-HPLC with the following conditions (2#SHIMADZU (HPLC-01)): Column, XBridge Prep OBD C18 Column, 30*150 mm Sum; mobile phase, Water (10 mmol/l NH₄HCO₃) and ACN (33% Phase B up to 60% in 7 min); Detector, uv.6-[4-[(2,2-difluoromorpholin-4-yl) methyl]phenyl]-4-[(3S)-piperidin-3-ylamino]pyrido[3,2-d]pyrimidine-8-carboxamide (11.9 mg, 39.34%) as a light yellow solid.

HPLC: 97% purity, RT=5.229 min. MS: m/z=484.2 [M+H]⁺. ¹H NMR (300 MHz, DMSO-d6, ppm) δ 10.05 (d, J=3.7 Hz, 1H), 8.84 (s, 1H), 8.57 (s, 1H), 8.35 (t, J=8.4 Hz, 3H), 8.22 (d, J=3.7 Hz, 1H), 7.52 (d, J=8.2 Hz, 2H), 4.34-4.25 (m, 1H), 4.08-3.99 (m, 2H), 3.72 (s, 2H), 3.09-2.99 (m, 1H), 2.88-2.71 (m, 4H), 2.58 (d, J=7.2 Hz, 3H), 1.86 (d, J=18.3 Hz, 2H), 1.69 (d, J=12.0 Hz, 1H), 1.58-1.42 (m, 1H).

### Example 71: 4-[[(3S,5S)-5-fluoropiperidin-3-yl]amino]-6-[4-(trifluoromethoxy) phenyl]pyrido[3,2-d]pyrimidine-8-carboxamide

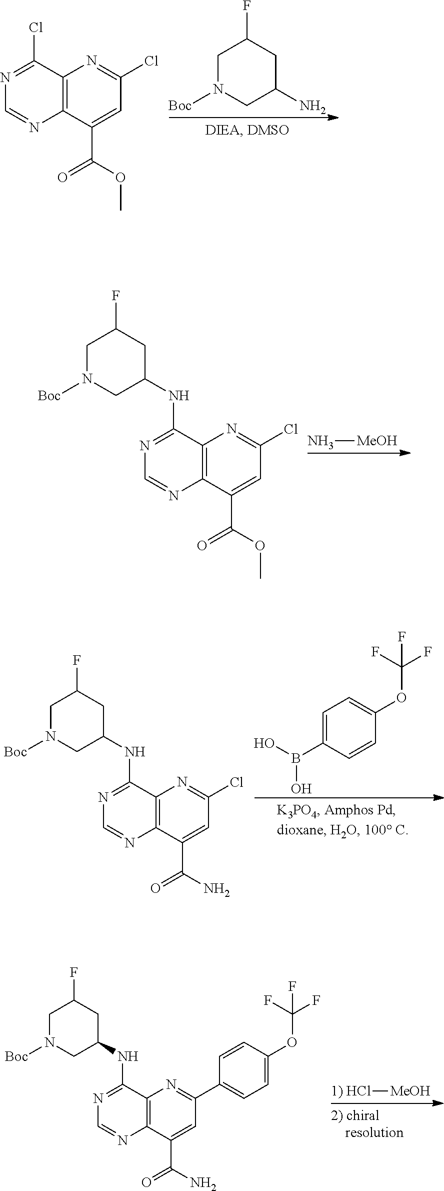

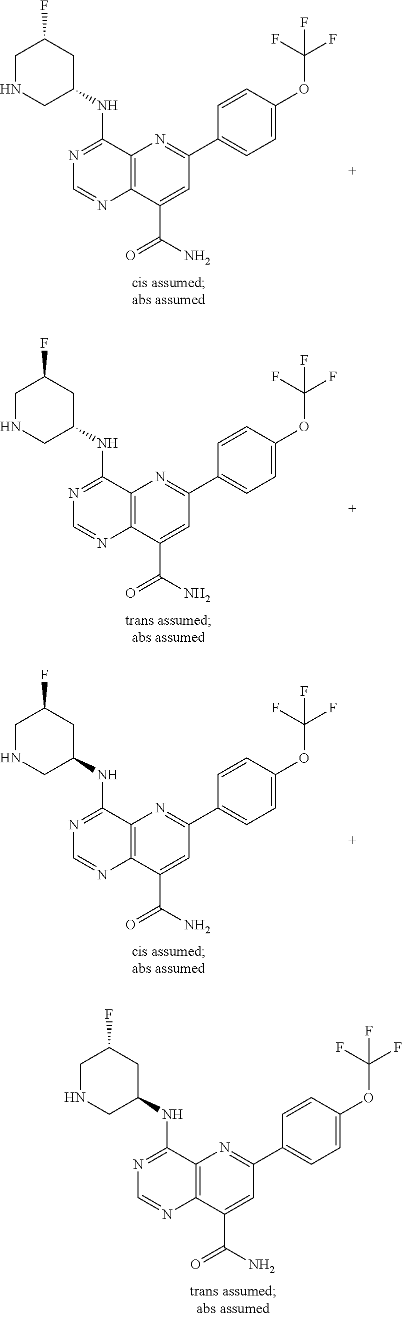 [SMILES: NC(=O)c1cc(-c2ccc(OC(F)(F)F)cc2)nc2c(N[C@@H]3CNC[C@H](F)C3)ncnc12 ; NC(=O)c1cc(-c2ccc(OC(F)(F)F)cc2)nc2c(N[C@@H]3CNC[C@@H](F)C3)ncnc12 ; NC(=O)c1cc(-c2ccc(OC(F)(F)F)cc2)nc2c(N[C@H]3CNC[C@@H](F)C3)ncnc12 ; NC(=O)c1cc(-c2ccc(OC(F)(F)F)cc2)nc2c(N[C@H]3CNC[C@H](F)C3)ncnc12]

tert-butyl 3-[[6-chloro-8-(methoxycarbonyl) pyrido[3,2-d]pyrimidin-4-yl]amino]-5-fluoropiperidine-1-carboxylate: To a stirred mixture of methyl 4,6-dichloropyrido[3,2-d]pyrimidine-8-carboxylate (1.00 g, 1.984 mmol) and tert-butyl 3-amino-5-fluoropiperidine-1-carboxylate (501.44 mg, 2.182 mmol) in DMSO (10.00 mL) was added DIEA (809.76 mg, 5.952 mmol). The resulting mixture was stirred for 3 h at 40° C. under nitrogen atmosphere. The reaction was quenched with Water at room temperature. The resulting mixture was extracted with EtOAc (4×50 mL). The combined organic layers were washed with brine (3×100 mL), dried over anhydrous Na₂SO₄. After filtration, the filtrate was concentrated under reduced pressure. This resulted in tert-butyl 3-[[6-chloro-8-(methoxycarbonyl) pyrido[3,2-d]pyrimidin-4-yl]amino]-5-fluoropiperidine-1-carboxylate (1 g, 97.40%) as a brown solid.

tert-butyl 3-([8-carbamoyl-6-chloropyrido[3,2-d]pyrimidin-4-yl]amino)-5-fluoropiperidine-1-carboxylate: A solution of tert-butyl 3-[[6-chloro-8-(methoxycarbonyl) pyrido[3,2-d]pyrimidin-4-yl]amino]-5-fluoropiperidine-1-carboxylate (1.00 g, 1.932 mmol) in NH₃ (g) in MeCOH (15.00 mL) was stirred for 3 h at 40° C. under nitrogen atmosphere. The resulting mixture was concentrated under vacuum. This resulted in tert-butyl 3-([8-carbamoyl-6-chloropyrido[3,2-d]pyrimidin-4-yl]amino)-5-fluoropiperidine-1-carboxylate (570 mg, 48.53%) as a yellow solid.

tert-butyl 3-([8-carbamoyl-6-[4-(trifluoromethoxy) phenyl]pyrido[3,2-d]pyrimidin-4-yl]amino)-5-fluoropiperidine-1-carboxylate: To a solution of tert-butyl 3-([8-carbamoyl-6-chloropyrido[3,2-d]pyrimidin-4-yl]amino)-5-fluoropiperidine-1-carboxylate (560.00 mg, 0.921 mmol) and 4-(trifluoromethoxy)phenylboronic acid (239.66 mg, 1.106 mmol) in dioxane (5.00 mL) and H₂O (1.00 mL) were added K₃PO₄ (617.59 mg, 2.764 mmol) and Pd(AMPHOS)Cl₂ (68.67 mg, 0.092 mmol). After stirring for 2 h at 100° C. under a nitrogen atmosphere, the resulting mixture was concentrated under reduced pressure. The residue was purified by silica gel column chromatography, eluted with PE/EtOAc (4:1) to afford tert-butyl 3-([8-carbamoyl-6-[4-(trifluoromethoxy) phenyl]pyrido[3,2-d]pyrimidin-4-yl]amino)-5-fluoropiperidine-1-carboxylate (360 mg, 63.52%) as a yellow solid.

4-[(5-fluoropiperidin-3-yl) amino]-6-[4-(trifluoromethoxy) phenyl]pyrido[3,2-d]pyrimidine-8-carboxamide: A solution of tert-butyl 3-([8-carbamoyl-6-[4-(trifluoromethoxy)phenyl]pyrido[3,2-d]pyrimidin-4-yl]amino)-5-fluoropiperidine-1-carboxylate (350.00 mg, 0.569 mmol) in HCl (g) in MeOH (10.00 mL) was stirred for 2 h at room temperature under nitrogen atmosphere. The resulting mixture was concentrated under vacuum. The crude product (230 mg) was purified by Prep-HPLC with the following conditions (2#SHIMADZU (HPLC-01)): Column, XSelect CSH Prep C18 OBD Column, 5 um, 19*150 mm; mobile phase, Water (10 MMOL/L NH₄HCO₃) and ACN (35% Phase B up to 65% in 7 min); Detector, UV. product was obtained. This resulted in 4-[(5-fluoropiperidin-3-yl) amino]-6-[4-(trifluoromethoxy) phenyl]pyrido[3,2-d]pyrimidine-8-carboxamide (95 mg, 37.07%) as a white solid.

4-[[(3S,5S)-5-fluoropiperidin-3-yl]amino]-6-[4-(trifluoromethoxy) phenyl]pyrido [3,2-d]pyrimidine-8-carboxamide: The product (95 mg) was purified by Chiral-Prep-HPLC with the following conditions (Prep-HPLC-009): Column, CHIRALPAK IE, 2*25 cm, 5 um; mobile phase, MTBE (10 mM NH₃-MeOH) and EtOH (hold 30% EtOH in 14 min); Detector, UV 254. This resulted in 4-[[(3S,5R)-5-fluoropiperidin-3-yl]amino]-6-[4-(trifluoromethoxy) phenyl]pyrido[3,2-d]pyrimidine-8-carboxamide (35.4 mg, 35.77%) as a light yellow solid; 4-[[(3S,5S)-5-fluoropiperidin-3-yl]amino]-6-[4-(trifluoromethoxy)phenyl]pyrido[3,2-d]pyrimidine-8-carboxamide (16.5 mg, 17.19%) as a white solid; 4-[[(3R,5S)-5-fluoropiperidin-3-yl]amino]-6-[4-(trifluoromethoxy) phenyl]pyrido[3,2-d]pyrimidine-8-carboxamide (27.6 mg, 28.94%) as a light yellow solid; 4-[[(3R,5R)-5-fluoropiperidin-3-yl]amino]-6-[4-(trifluoromethoxy) phenyl]pyrido[3,2-d]pyrimidine-8-carboxamide (21.0 mg, 21.97%) as a white solid.

4-[[(3S,5R)-5-fluoropiperidin-3-yl]amino]-6-[4-(trifluoromethoxy) phenyl]pyrido [3,2-d]pyrimidine-8-carboxamide: HPLC: 94% purity, RT=7.068 min. MS:m/z=451.1[M+H]⁺. ¹H NMR (400 MHz, DMSO) δ 9.96 (d, J=3.5 Hz, 1H), 8.86 (t, J=2.2 Hz, 1H), 8.61 (t, J=2.0 Hz, 1H), 8.55 (s, 1H), 8.56-8.43 (m, 2H), 8.26 (d, J=3.6 Hz, 1H), 7.54 (d, J=8.3 Hz, 2H), 4.89-4.77 (m, 1H), 4.50 (m, J=8.6, 4.3 Hz, 1H), 3.26-3.06 (m, 2H), 3.01-2.81 (m, 2H), 2.36 (m, 1H), 2.21-2.07 (m, 1H).

4-[[(3S,5S)-5-fluoropiperidin-3-yl]amino]-6-[4-(trifluoromethoxy) phenyl]pyrido [3,2-d]pyrimidine-8-carboxamide: HPLC: 98% purity, RT=4.454 min. MS:m/z=451.1[M+H]⁺. ¹H NMR (400 MHz, DMSO) δ 10.01 (d, J=3.6 Hz, 1H), 8.83 (q, J=1.6 Hz, 1H), 8.57 (d, J=1.6 Hz, 1H), 8.48 (m, J=9.0, 2.3 Hz, 2H), 8.41 (d, J=8.8 Hz, 1H), 8.23 (d, J=3.7 Hz, 1H), 7.51 (d, J=8.2 Hz, 2H), 4.80-4.61 (m, 1H), 4.60-4.57 (m, 1H), 3.03 (m, 2H), 2.81-2.64 (m, 2H), 2.25-2.05 (m, 2H).

4-[[(3R,5S)-5-fluoropiperidin-3-yl]amino]-6-[4-(trifluoromethoxy) phenyl]pyrido[3,2-d]pyrimidine-8-carboxamide: HPLC: 97% purity, RT=9.560 min. MS:m/z=451.1[M+H]⁺. ¹H NMR (400 MHz, DMSO) b 9.96 (d, J=3.5 Hz, 1H), 8.86 (t, J=2.2 Hz, 1H), 8.61 (t, J=2.0 Hz, 1H), 8.55 (s, 1H), 8.56-8.43 (m, 2H), 8.26 (d, J=3.6 Hz, 1H), 7.54 (d, J=8.3 Hz, 2H), 4.89-4.77 (m, 1H), 4.50 (m, J=8.6, 4.3 Hz, 1H), 3.26-3.06 (m, 2H), 3.01-2.81 (m, 2H), 2.36 (m, 1H), 2.21-2.07 (m, 1H).

4-[[(3R,5R)-5-fluoropiperidin-3-yl]amino]-6-[4-(trifluoromethoxy) phenyl]pyrido[3,2-d]pyrimidine-8-carboxamide: HPLC: 99% purity, RT=4.459 min. MS:m/z=451.1[M+H]⁺. ¹H NMR (400 MHz, DMSO) δ 10.01 (d, J=3.6 Hz, 1H), 8.83 (q, J=1.6 Hz, 1H), 8.57 (d, J=1.6 Hz, 1H), 8.48 (m, J=9.0, 2.3 Hz, 2H), 8.41 (d, J=8.8 Hz, 1H), 8.23 (d, J=3.7 Hz, 1H), 7.51 (d, J=8.2 Hz, 2H), 4.80-4.61 (m, 1H), 4.60-4.57 (m, 1H), 3.03 (m, 2H), 2.81-2.64 (m, 2H), 2.25-2.05 (m, 2H).

### Example 72: 6-(2-methoxyphenyl)-4-[(3S)-piperidin-3-ylamino]pyrido[3,2-d]pyrimidine-8-carboxamide

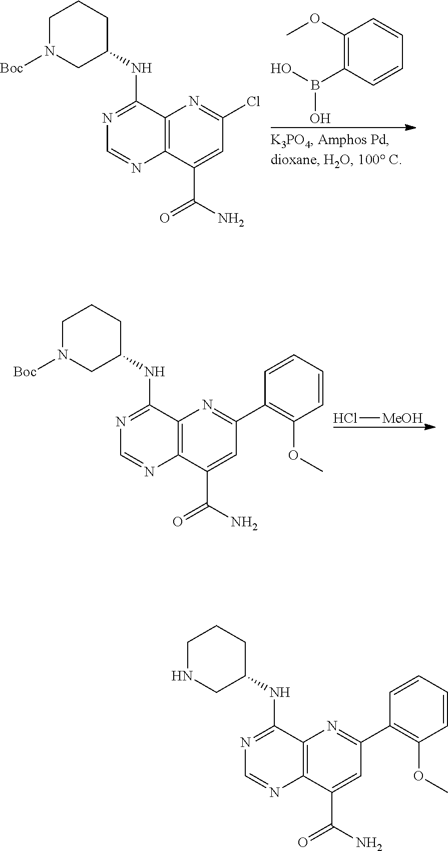

tert-butyl (3S)-3-[[8-carbamoyl-6-(2-methoxyphenyl)pyrido[3,2-d]pyrimidin-4-yl]amino]piperidine-1-carboxylate: To a solution of tert-butyl (3S)-3-([8-carbamoyl-6-chloropyrido[3,2-d]pyrimidin-4-yl]amino) piperidine-1-carboxylate (85.00 mg, 0.128 mmol) and 2-methoxyphenylboronic acid (38.00 mg, 0.238 mmol) in dioxane (5.00 mL) and H₂O (1.00 mL) were added K₃PO₄ (133.00 mg, 0.595 mmol) and A-Phos-PdCl2 (15.00 mg, 0.020 mmol). After stirring for 2 h at 80° C. under a nitrogen atmosphere, the resulting mixture was concentrated under reduced pressure. The residue was purified by silica gel column chromatography, eluted with PE/EtOAc (1:1) to afford tert-butyl (3S)-3-[[8-carbamoyl-6-(2-methoxyphenyl) pyrido [3,2-d]pyrimidin-4-yl]amino]piperidine-1-carboxylate (70 mg, 83.91%) as a yellow solid.

6-(2-methoxyphenyl)-4-[(3S)-piperidin-3-ylamino]pyrido[3,2-d]pyrimidine-8-carboxamide: A solution of tert-butyl (3S)-3-[[8-carbamoyl-6-(2-methoxyphenyl) pyrido [3,2-d]pyrimidin-4-yl]amino]piperidine-1-carboxylate (70.00 mg, 0.107 mmol) in HCl (g) in MeOH (8.00 mL) was stirred for 2 h at room temperature. The crude product was purified by Prep-HPLC with the following conditions (2#SHIMADZU (HPLC-01)): Column, XBridge Shield RP18 OBD Column, 5 um, 19*150 mm; mobile phase, Water (10 mmol/l NH₄HCO₃+0.05% NH₄OH) and ACN (32% Phase B up to 62% in 8 min); Detector, uv. 6-(2-methoxyphenyl)-4-[(3S)-piperidin-3-ylamino]pyrido[3,2-d]pyrimidine-8-carboxamide (26 mg, 61.64%) was obtained.

HPLC: 95% purity, RT=1.499 min. MS: m/z=379.2 [M+H]⁺. ¹H NMR (300 MHz, DMSO-d6, ppm) 10.07 (s, 1H), 8.80 (s, 1H), 8.57 (s, 1H), 8.26 (d, J=8.5 Hz, 1H), 8.18 (s, 1H), 7.91 (dd, J=7.6, 1.8 Hz, 1H), 7.50 (td, J=7.9, 7.3, 1.8 Hz, 1H), 7.22 (d, J=8.3 Hz, 1H), 7.14 (t, J=7.5 Hz, 1H), 4.27 (s, 1H), 3.87 (s, 3H), 3.00 (d, J=11.6 Hz, 1H), 2.74 (s, 2H), 1.81 (d, J=12.3 Hz, 2H), 1.55 (d, J=47.3 Hz, 2H).

### Example 73: 6-(1-methyl-1,2,3,6-tetrahydropyridin-4-yl)-4-{[(3S)-piperidin-3-yl]amino}pyrido[3,2-d]pyrimidine-8-carboxamide

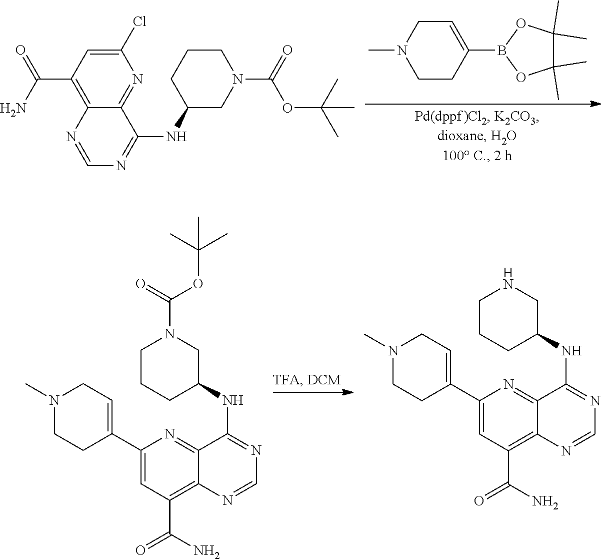

tert-butyl (3S)-3-{[8-carbamoyl-6-(1-methyl-1,2,3,6-tetrahydropyridin-4-yl)pyrido[3,2-d]pyrimidin-4-yl]amino}piperidine-1-carboxylate: tert-butyl (3S)-3-({8-carbamoyl-6-chloropyrido[3,2-d]pyrimidin-4-yl}amino)piperidine-1-carboxylate (50.0 mg; 0.123 mmol; 1.00 eq.), 1-methyl-4-(4,4,5,5-tetramethyl-1,3,2-dioxaborolan-2-yl)-1,2,3,6-tetrahydropyridine (32.9 mg; 0.147 mmol; 1.20 eq.), potassium carbonate (51.0 mg; 0.369 mmol; 3.00 eq.), Pd(dppf)Cl₂ (5.0 mg; 0.006 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h

The reaction mixture was filtered through celite, concentrated under reduced pressure to give tert-butyl (3S)-3-{[8-carbamoyl-6-(1-methyl-1 2,3,6-tetrahydropyridin-4-yl)pyrido[3,2-d]pyrimidin-4-yl]amino}piperidine-1-carboxylate (52.0 mg; 0.111 mmol; 90.5%).

6-(1-methyl-1,2,3,6-tetrahydropyridin-4-yl)-4-{[(3S)-piperidin-3-yl]amino}pyrido[3,2-d]pyrimidine-8-carboxamide: tert-butyl (3S)-3-{[8-carbamoyl-6-(1-methyl-1,2,3,6-tetrahydropyridin-4-yl)pyrido[3,2-d]pyrimidin-4-yl]amino}piperidine-1-carboxylate (52.0 mg; 0.111 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The product was purified on waters reverse phase system using a gradient of 10-60% CH₃CN/H₂O (0.1% Ammonium Hydroxide) in 3 injections. The desired fractions were evaporated with Genevac to give 6-(1-methyl-1,2,3,6-tetrahydropyridin-4-yl)-4-{[(3S)-piperidin-3-yl]amino}pyrido[3,2-d]pyrimidine-8-carboxamide (17.0 mg; 0.046 mmol; 41.6%) as a white solid.

¹H NMR (400 MHz, Methanol-d4) δ 8.60 (s, 1H), 8.47 (s, 1H), 6.91 (s, 1H), 4.35 (s, 1H), 3.35 (d, J=15.2 Hz, 3H), 3.04 (d, J=12.8 Hz, 1H), 2.80 (t, J=22.8 Hz, 6H), 2.47 (s, 3H), 2.14 (d, J=11.2 Hz, 1H), 1.92-1.68 (m, 3H).

LC/MS: [M+H] for C₁₉H₂₆N₇O calculated. 367.1 found. 367.2.

HPLC: 100% pure (254 nM) at 0:60 minutes

### Example 74: 6-[4-methyl-4-(morpholin-4-yl)cyclohex-1-en-1-yl]-4-{[(3S)-piperidin-3-yl]amino}pyrido[3,2-d]pyrimidine-8-carboxamide

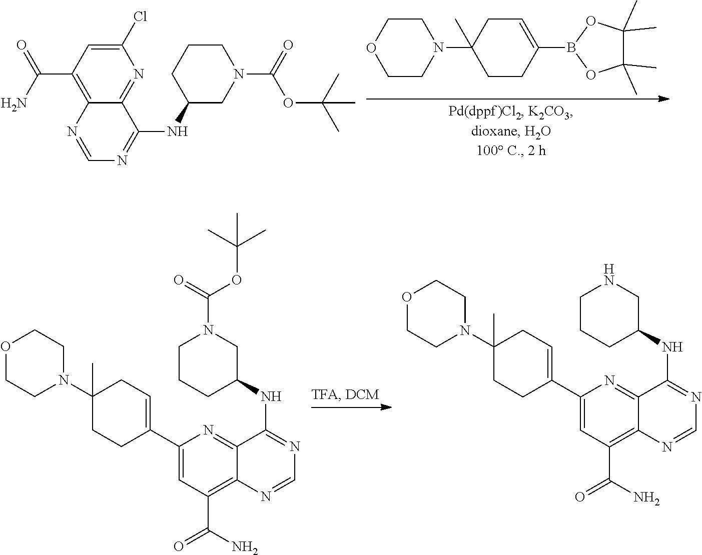

tert-butyl (3S)-3-({8-carbamoyl-6-[4-methyl-4-(morpholin-4-yl)cyclohex-1-en-1-yl]pyrido[3,2-d]pyrimidin-4-yl}amino)piperidine-1-carboxylate: tert-butyl (3S)-3-({8-carbamoyl-6-chloropyrido[3,2-d]pyrimidin-4-yl}amino)piperidine-1-carboxylate (50.0 mg; 0.123 mmol; 1.00 eq.), 4-[1-methyl-4-(4,4,5,5-tetramethyl-1,3,2-dioxaborolan-2-yl)cyclohex-3-en-1-yl]morpholine (45.3 mg; 0.147 mmol; 1.20 eq.), potassium carbonate (51.0 mg; 0.369 mmol; 3.00 eq.), Pd(dppf)Cl₂ (5.0 mg; 0.006 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h

The reaction mixture was filtered through celite, concentrated under reduced pressure to give tert-butyl (3S)-3-({8-carbamoyl-6-[4-methyl-4-(morpholin-4-yl)cyclohex-1-en-1-yl]pyrido[3,2-d]pyrimidin-4-yl}amino)piperidine-1-carboxylate (66.0 mg; 0.120 mmol; 97.4%) crude.

6-[4-methyl-4-(morpholin-4-yl)cyclohex-1-en-1-yl]-4-{[(3S)-piperidin-3-yl]amino}pyrido[3,2-d]pyrimidine-8-carboxamide: tert-butyl (3S)-3-({8-carbamoyl-6-[4-methyl-4-(morpholin-4-yl)cyclohex-1-en-1-yl]pyrido[3,2-d]pyrimidin-4-yl}amino)piperidine-1-carboxylate (66.0 mg; 0.120 mmol; 1.00 eq.) was dissolved in dichloromethane (1.0 ml). TFA (0.3 ml) was added to reaction mixture. The resulting solution was stirred for two hours. The volatiles were evaporated and the residue dissolved in 3 mL of DMSO. The product was purified on waters reverse phase system using a gradient of 10-60% CH₃CN/H₂O (0.1% Ammonium Hydroxide) in 3 injections. The desired fractions were evaporated with Genevac to give 6-[4-methyl-4-(morpholin-4-yl)cyclohex-1-en-1-yl]-4-{[(3S)-piperidin-3-yl]amino}pyrido[3,2-d]pyrimidine-8-carboxamide (23.0 mg; 0.051 mmol; 42.6%) as a white solid.

¹H NMR (400 MHz, Methanol-d4) δ 8.59 (s, 1H), 8.46 (s, 1H), 6.83 (s, 1H), 4.31 (s, 1H), 3.72 (d, J=4.3 Hz, 4H), 3.28 (d, J=11.7 Hz, 1H), 3.08-2.41 (m, 11H), 2.25 (d, J=18.4 Hz, 1H), 2.12 (d, J=8.2 Hz, 1H), 1.97-1.60 (m, 5H), 1.06 (s, 3H).

LC/MS: [M+H] for C₂₄H₃₄N₇O₂ calculated. 452.1 found. 452.0.

HPLC: 100% pure (254 nM) at 1:40 minutes

### Example 75: 6-(1-cyclopropanecarbonyl-1,2,3,6-tetrahydropyridin-4-yl)-4-{[(3S)-piperidin-3-yl]amino}pyrido[3,2-d]pyrimidine-8-carboxamide

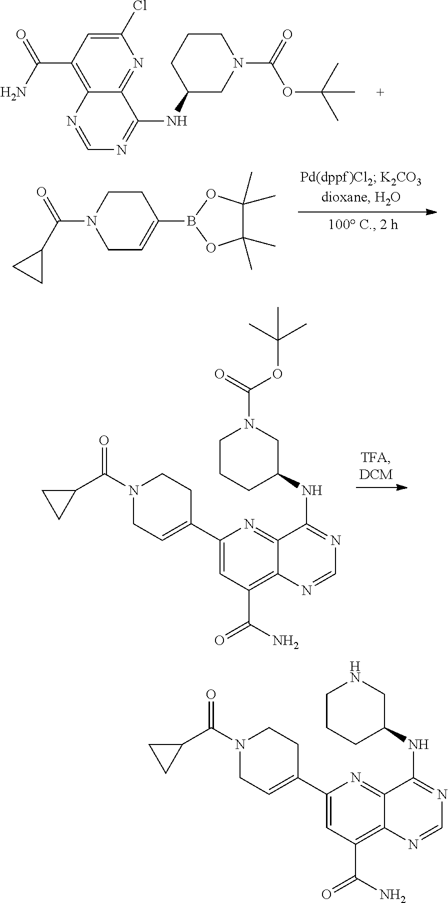

tert-butyl (3S)-3-{[8-carbamoyl-6-(1-cyclopropanecarbonyl-1,2,3,6-tetrahydropyridin-4-yl)pyrido[3,2-d]pyrimidin-4-yl]amino}piperidine-1-carboxylate: tert-butyl (3S)-3-({8-carbamoyl-6-chloropyrido[3,2-d]pyrimidin-4-yl}amino)piperidine-1-carboxylate (50.0 mg; 0.123 mmol; 1.00 eq.), cyclopropyl(4-(4,4,5,5-tetramethyl-1,3,2-dioxaborolan-2-yl)-5,6-dihydropyridin-1 (2h)-yl)methanone (40.9 mg; 0.147 mmol; 1.20 eq.), potassium carbonate (51.0 mg; 0.369 mmol; 3.00 eq.), Pd(dppf)Cl₂ iron (5.0 mg; 0.006 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h. The reaction mixture was filtered through celite, concentrated under reduced pressure, to give tert-butyl (3S)-3-{[8-carbamoyl-6-(1-cyclopropanecarbonyl-1,2,3,6-tetrahydropyridin-4-yl)pyrido[3,2-d]pyrimidin-4-yl]amino}piperidine-1-carboxylate (60.0 mg; 0.115 mmol; 93.6%) as an off white solid.

6-(1-cyclopropanecarbonyl-1,2,3,6-tetrahydropyridin-4-yl)-4-{[(3S)-piperidin-3-yl]amino}pyrido[3,2-d]pyrimidine-8-carboxamide: tert-butyl (3S)-3-{[8-carbamoyl-6-(1-cyclopropanecarbonyl-1,2,3,6-tetrahydropyridin-4-yl)pyrido[3,2-d]pyrimidin-4-yl]amino}piperidine-1-carboxylate (60.0 mg; 0.115 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The product was purified on waters reverse phase system using a gradient of 10-60% CH₃CN/H₂O (0.1% Ammonium Hydroxide) in 3 injections. The desired fractions were evaporated with Genevac to give 6-(1-cyclopropanecarbonyl-1,2,3,6-tetrahydropyridin-4-yl)-4-{[(3S)-piperidin-3-yl]amino}pyrido[3,2-d]pyrimidine-8-carboxamide (2.0 mg; 0.005 mmol; 4.1%) as a white solid.

¹H NMR (400 MHz, Methanol-d4) δ 8.59 (d, J=10.2 Hz, 1H), 8.47 (s, 1H), 6.90 (s, 1H), 4.58 (s, 1H), 4.34 (s, 2H), 3.95 (d, J=66.0 Hz, 2H), 3.28 (d, J=11.6 Hz, 1H), 3.03-2.63 (m, 5H), 2.06 (d, J=38.7 Hz, 2H), 1.91-1.59 (m, 3H), 0.92 (d, J=26.6 Hz, 4H).

LC/MS: [M+H] for C₂₂H₂₈N₇O₂ calculated. 422.1 found. 422.2.

HPLC: 96% pure (254 nM) at 1:93 minutes

### Example 76: 6-(4-fluorophenyl)-4-{[(3S)-piperidin-3-yl]amino}pyrido[3,2-d]pyrimidine-8-carboxamide

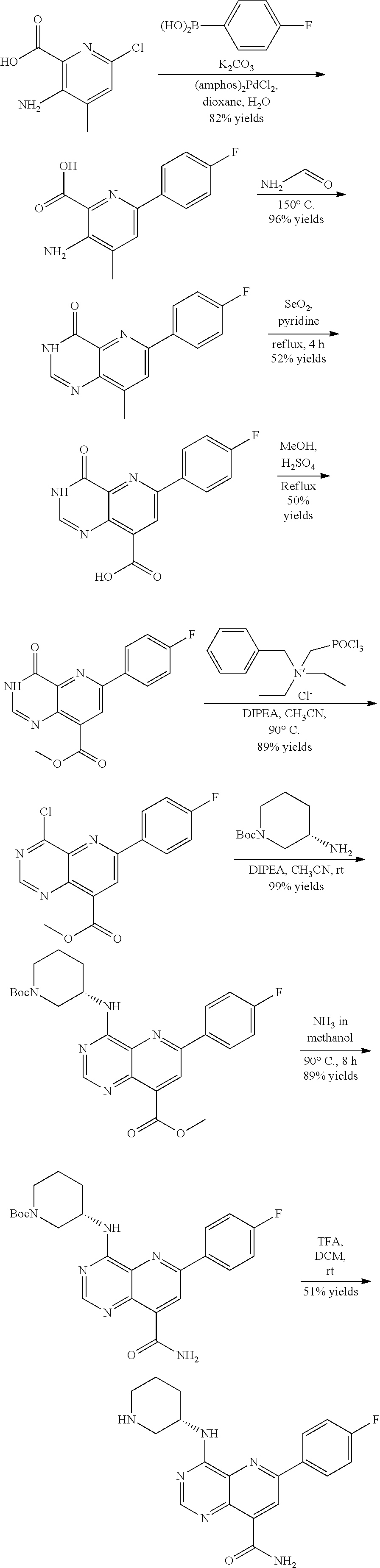

3-amino-6-(4-fluorophenyl)-4-methylpyridine-2-carboxylic acid: 3-amino-6-chloro-4-methylpicolinic acid (1000.0 mg; 5.359 mmol; 1.00 eq.), 4-fluorophenylboronic acid (1124.8 mg; 8.039 mmol; 1.50 eq.), potassium carbonate (2222.0 mg; 16.078 mmol; 3.00 eq.), bis(di-tert-butyl(4-dimethylaminophenyl)phosphine)dichloropalladium(ii) (1138.4 mg; 1.608 mmol; 0.30 eq.), Dioxane (10.0 ml) and water (2.0 ml) were added to a microwave vial. The tube was sealed and flushed with nitrogen for 5 min and the suspension was microwaved at 100° C. for 4 h. LCMS showed 100% conversion. Water 1 mL was added and the solid filtered off. The mother liquid was acidified with 1 N HCl and the precipitated solid filtered, washed with hexane (10 mL), dried and collected to give 3-amino-6-(4-fluorophenyl)-4-methylpyridine-2-carboxylic acid (967.0 mg; 3.927 mmol; 73.3%) as a yellow solid.

6-(4-fluorophenyl)-8-methylpyrido[3,2-d]pyrimidin-4-ol: 3-amino-6-(4-fluorophenyl)-4-methylpyridine-2-carboxylic acid (967.0 mg; 3.927 mmol; 1.00 eq.), formamide (15.0 ml), and were added to a vial. The tube was sealed and flushed with nitrogen for 5 min and the suspension was at 140° C. for 6 h. The desired crashed out. Water (2 mL) was added, and the suspension filtered. The solid washed with water (2 mL) then hexane (5 mL). The solid was collected and dried to give 6-(4-fluorophenyl)-8-methylpyrido[3,2-d]pyrimidin-4-ol (672.0 mg; 2.633 mmol; 67.0%) as a yellow solid.

6-(4-fluorophenyl)-4-hydroxypyrido[3,2-d]pyrimidine-8-carboxylic acid: 6-(4-fluorophenyl)-8-methylpyrido[3,2-d]pyrimidin-4-ol (500.0 mg; 1.959 mmol; 1.00 eq.), pyridine (10.0 ml) and selenium dioxide (869.4 mg; 7.836 mmol; 4.00 eq.) were added to a vial. The suspension was heated at 100° C. for 4 h. LCMS showed the reaction was complete. The solid was filtered off and the mother liquid evaporated to give 6-(4-fluorophenyl)-4-hydroxypyrido[3,2-d]pyrimidine-8-carboxylic acid (480.0 mg; 0.673 mmol; 34.4%) crude.

methyl 6-(4-fluorophenyl)-4-hydroxypyrido[3,2-d]pyrimidine-8-carboxylate: 6-(4-fluorophenyl)-4-hydroxypyrido[3,2-d]pyrimidine-8-carboxylic acid (480.0 mg; 1.346 mmol; 1.00 eq.) was dissolved in methanol (2.7 ml; 1.346 mmol; 1.00 eq.) before sulfuric acid (84.1 μl; 1.346 mmol; 1.00 eq.) was added. The solution was then heated for 2 hour at 100° C. LCMS showed complete conversion. The solution was then evaporated, and the residue dissolved in DMSO. It was purified with the interchim system (water/acetonitrile 90:10 to 0:100 over 20 minutes) to give methyl 6-(4-fluorophenyl)-4-hydroxypyrido[3,2-d]pyrimidine-8-carboxylate (299.0 mg; 0.909 mmol; 67.5%)

methyl 4-chloro-6-(4-fluorophenyl)pyrido[3,2-d]pyrimidine-8-carboxylate: In a 250 ml round bottom flask, a suspension of methyl 6-(4-fluorophenyl)-4-hydroxypyrido[3,2-d]pyrimidine-8-carboxylate (299.0 mg; 0.909 mmol; 1.00 eq.) and benzyltriethylazanium chloride (269.2 mg; 1.182 mmol; 1.30 eq.) in toluene/acetonitrile 1/1 (100 ml, sonicated for 5 minutes) was evaporated under high vacuum to dryness (3 mbarr, rotavap water bath temperature 45° C. for 15 min). Dry acetonitrile (10.0 ml), was added, the flask was equipped with a rubber septum and evacuated with vacuum and filled with dry argon three times. Under argon, the suspension was treated with phosphoroyl trichloride (250.4 μl; 2.728 mmol; 3.00 eq.) via syringe over a period of 2 minutes, stirred at room temperature for an additional 5 minutes then Hunig's base ethylbis(propan-2-yl)amine (162.4 μl; 0.955 mmol; 1.05 eq.) was added via syringe at once. The heating block was then set to 90° C. and the contents were stirred for 2 h. LC-MS analysis indicated the reaction was complete (use dry non-protic solvents such as acetonitrile for HPLC sample preparation). The reaction mixture was evaporated and the residue dried under high vacuum to afford the desired crude product methyl 4-chloro-6-(4-fluorophenyl)pyrido[3,2-d]pyrimidine-8-carboxylate (267.0 mg; 0.840 mmol; 92.4%).

tert-butyl (3S)-3-{[6-(4-fluorophenyl)-8-(methoxycarbonyl)pyrido[3,2-d]pyrimidin-4-yl]amino}piperidine-1-carboxylate: methyl-4-chloro-6-(4-fluorophenyl)pyrido[3,2-d]pyrimidine-8-carboxylate (287.0 mg; 0.723 mmol; 1.00 eq.) and (S)-3-Amino-piperidine-1-carboxylic acid tert-butyl ester (173.7 mg; 0.867 mmol; 1.20 eq.) stirred in dioxane with DIPEA at rt overnight. LCMS showed major peak was for desired product. It was filtered through silica gel. The filtrate was evaporated to give tert-butyl (3S)-3-{[6-(4-fluorophenyl)-8-(methoxycarbonyl)pyrido[3,2-d]pyrimidin-4-yl]amino}piperidine-1-carboxylate (217.0 mg; 0.406 mmol; 56.1%) as a yellow solid.

tert-butyl (3S)-3-{[8-carbamoyl-6-(4-fluorophenyl)pyrido[3,2-d]pyrimidin-4-yl]amino}piperidine-1-carboxylate: tert-butyl (3S)-3-{[6-(4-fluorophenyl)-8-(methoxycarbonyl)pyrido[3,2-d]pyrimidin-4-yl]amino}piperidine-1-carboxylate (90.0 mg; 0.187 mmol; 1.00 eq.) was suspended in 7 N ammonia (1.0 ml; 7.000 mmol; 37.45 eq.) in a 5 ml microwave tube and stirred in microwave at 90° C. for 8 hours. LCMS showed the desired product as major product. The volatiles was rotovaped to dryness and the crude carried to the next step without further purification tert-butyl (3S)-3-{[8-carbamoyl-6-(4-fluorophenyl)pyrido[3,2-d]pyrimidin-4-yl]amino}piperidine-1-carboxylate (85.0 mg; 0.182 mmol; 97.5%).

6-(4-fluorophenyl)-4-{[(3S)-piperidin-3-yl]amino}pyrido[3,2-d]pyrimidine-8-carboxamide: tert-butyl (3S)-3-{[8-carbamoyl-6-(4-fluorophenyl)pyrido[3,2-d]pyrimidin-4-yl]amino}piperidine-1-carboxylate (85.0 mg; 0.182 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 2 mL of DMSO. The product was purified on waters reverse phase system using a gradient of 10-50% CH₃CN/H₂O (0.1% Ammonium Hydroxide) in 2 injections. The desired fractions were evaporated with Genevac to give 6-(4-fluorophenyl)-4-{[(3S)-piperidin-3-yl]amino}pyrido[3,2-d]pyrimidine-8-carboxamide (20.4 mg; 0.055 mmol; 30.3%) as a yellow solid.

¹H NMR (400 MHz, DMSO-d6) 10.01 (s, 1H), 8.83 (s, 1H), 8.58 (s, 1H), 8.51-8.34 (m, 3H), 8.21 (s, 1H), 7.47-7.31 (m, 2H), 4.35 (d, J=9.8 Hz, 1H), 3.10 (d, J=11.9 Hz, 1H), 2.88 (d, J=12.3 Hz, 1H), 2.79 (t, J=10.3 Hz, 1H), 2.60 (t, J=10.9 Hz, 1H), 1.92 (s, 1H), 1.84-1.69 (m, 2H), 1.54 (t, J=12.0 Hz, 1H).

LC/MS: [M+H]⁺ for C₁₉H₂₀FN₆O calculated. 367.1 found. 367.1.

HPLC: 99% pure (254 nM) at 2:60 minutes.

### Example 77: 6-[1-(difluoromethyl)-1H-pyrazol-4-yl]-4-{[(3S)-piperidin-3-yl]amino}pyrido[3,2-d]pyrimidine-8-carboxamide

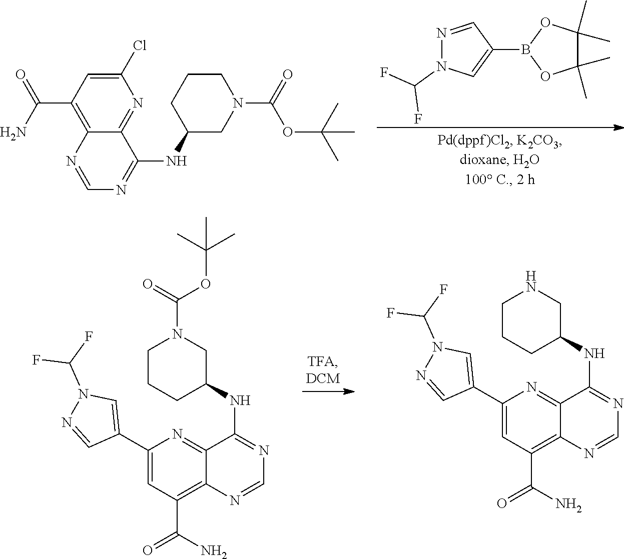 [SMILES: CC(C)(C)OC(=O)N1CCC[C@H](Nc2ncnc3c(C(N)=O)cc(Cl)nc23)C1 ; CC1(C)OB(c2cnn(C(F)F)c2)OC1(C)C ; CC(C)(C)OC(=O)N1CCC[C@H](Nc2ncnc3c(C(N)=O)cc(-c4cnn(C(F)F)c4)nc23)C1 ; NC(=O)c1cc(-c2cnn(C(F)F)c2)nc2c(N[C@H]3CCCNC3)ncnc12]

tert-butyl (3S)-3-({8-carbamoyl-6-[1-(difluoromethyl)-1H-pyrazol-4-yl]pyrido[3,2-d]pyrimidin-4-yl}amino)piperidine-1-carboxylate: tert-butyl (3S)-3-({8-carbamoyl-6-chloropyrido[3,2-d]pyrimidin-4-yl}amino)piperidine-1-carboxylate (50.0 mg; 0.123 mmol; 1.00 eq.), 1-(difluoromethyl)-4-(4,4,5,5-tetramethyl-1,3,2-dioxaborolan-2-yl)-1h-pyrazole (44.9 mg; 0.180 mmol; 1.50 eq.), potassium carbonate (51.0 mg; 0.369 mmol; 3.00 eq.), cyclopentyl(diphenyl)phosphane; dichloromethane; dichloropalladium; iron (5.0 mg; 0.006 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h

The reaction mixture was filtered through celite, concentrated under reduced pressure to give tert-butyl (3S)-3-({8-carbamoyl-6-[1-(difluoromethyl)-1H-pyrazol-4-yl]pyrido[3,2-d]pyrimidin-4-yl}amino)piperidine-1-carboxylate (60.0 mg; 0.12 mmol; 99.9%).

6-[1-(difluoromethyl)-1H-pyrazol-4-yl]-4-{[(3S)-piperidin-3-yl]amino}pyrido[3,2-d]pyrimidine-8-carboxamide: tert-butyl (3S)-3-({8-carbamoyl-6-[1-(difluoromethyl)-1H-pyrazol-4-yl]pyrido[3,2-d]pyrimidin-4-yl}amino)piperidine-1-carboxylate (60.0 mg; 0.12 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The sample was purified on an Agilent 1100 with an Aurora Fusion A5 SFC (ODH, 4.6×150 mm, particle size of 5 um, pore size 1000 A) with a column temperature of 40 C and mobile phases A: CO₂ B: Methanol+20 mM NH₄OH. Separation was achieved using a gradient of 85% solvent A+15% solvent B to 55% solvent A+45% solvent B. The desired fractions were combined and dried down via TurboVap using nitrogen flow to give 6-[1-(difluoromethyl)-1H-pyrazol-4-yl]-4-{[(3S)-piperidin-3-yl]amino}pyrido[3,2-d]pyrimidine-8-carboxamide (11.0 mg; 0.03 mmol; 22.1%) as a yellow solid.

¹H NMR (400 MHz, DMSO) δ 9.88 (d, J=3.5 Hz, 1H), 9.29 (s, 1H), 8.81 (s, 1H), 8.72 (s, 1H), 8.61 (s, 1H), 8.50 (d, J=8.4 Hz, 1H), 8.21 (s, 1H), 7.92 (t, J=59.2 Hz, 1H), 4.72-4.60 (m, 1H), 3.47 (dd, J=12.3, 4.5 Hz, 1H), 3.17 (s, 1H), 3.12 (t, J=11.6 Hz, 1H), 2.86 (t, J=11.9 Hz, 1H), 2.11-1.72 (m, 5H).

LC/MS: [M+H] for C₁₇H₁₈F₂N₈O calculated. 388.16 found. 389.2.

HPLC: 96% pure (254 nM) at 1:61 minutes.

### Example 78: 4-{[(3S)-piperidin-3-yl]amino}-6-[1-(propan-2-yl)-1H-pyrazol-4-yl]pyrido[3,2-d]pyrimidine-8-carboxamide

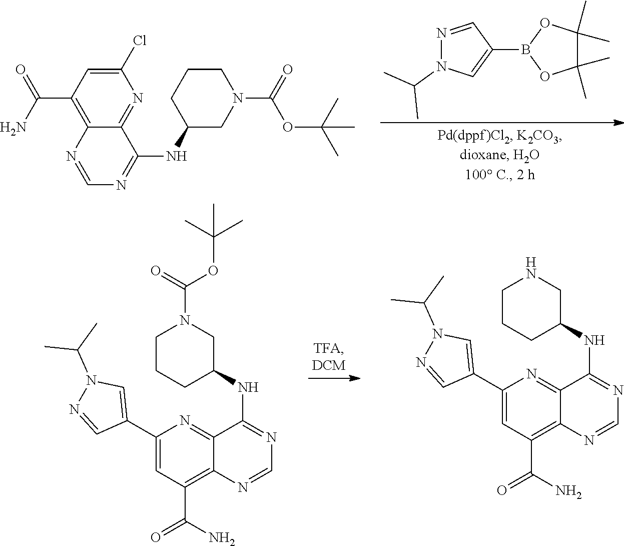 [SMILES: CC(C)(C)OC(=O)N1CCC[C@H](Nc2ncnc3c(C(N)=O)cc(Cl)nc23)C1 ; CC(C)n1cc(B2OC(C)(C)C(C)(C)O2)cn1 ; CC(C)n1cc(-c2cc(C(N)=O)c3ncnc(N[C@H]4CCCN(C(=O)OC(C)(C)C)C4)c3n2)cn1 ; CC(C)n1cc(-c2cc(C(N)=O)c3ncnc(N[C@H]4CCCNC4)c3n2)cn1]

tert-butyl (3S)-3-({8-carbamoyl-6-[1-(propan-2-yl)-1H-pyrazol-4-yl]pyrido[3,2-d]pyrimidin-4-yl}amino)piperidine-1-carboxylate: tert-butyl (3S)-3-({8-carbamoyl-6-chloropyrido[3,2-d]pyrimidin-4-yl}amino)piperidine-1-carboxylate (50.0 mg; 0.123 mmol; 1.00 eq.), 1-isopropyl-4-(4,4,5,5-tetramethyl-1,3,2-dioxaborolan-2-yl)-1h-pyrazole (45.5 mg; 0.18 mmol; 1.50 eq.), potassium carbonate (51.0 mg; 0.369 mmol; 3.00 eq.), Pd(dppf)Cl₂ (5.0 mg; 0.006 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h

The reaction mixture was filtered through celite, concentrated under reduced pressure to give tert-butyl (3S)-3-({8-carbamoyl-6-[1-(propan-2-yl)-1H-pyrazol-4-yl]pyrido[3,2-d]pyrimidin-4-yl}amino)piperidine-1-carboxylate (59.0 mg; 0.120 mmol; 99.9%) crude.

4-{[(3S)-piperidin-3-yl]amino}-6-[1-(propan-2-yl)-1H-pyrazol-4-yl]pyrido[3,2-d]pyrimidine-8-carboxamide: tert-butyl (3S)-3-({8-carbamoyl-6-[1-(propan-2-yl)-1H-pyrazol-4-yl]pyrido[3,2-d]pyrimidin-4-yl}amino)piperidine-1-carboxylate (59.0 mg; 0.120 mmol; 1.00 eq.) was dissolved in dichloromethane (1.0 ml). TFA (0.3 ml) was added to reaction mixture. The resulting solution was stirred for two hours. The volatiles were evaporated and the residue dissolved in 3 mL of DMSO. The sample was purified on an Agilent 1100 with an Aurora Fusion A5 SFC (ODH, 4.6×150 mm, particle size of 5 um, pore size 1000 A) with a column temperature of 40 C and mobile phases A: CO₂ B: Methanol+20 mM NH₄OH. Separation was achieved using a gradient of 85% solvent A+15% solvent B to 55% solvent A+45% solvent B. The desired fractions were combined and dried down via TurboVap using nitrogen flow to give 4-{[(3S)-piperidin-3-yl]amino}-6-[1-(propan-2-yl)-1H-pyrazol-4-yl]pyrido[3,2-d]pyrimidine-8-carboxamide (35.0 mg; 0.09 mmol; 70.4%) as a yellow oil.

¹H NMR (400 MHz, DMSO) δ 9.93 (d, J=3.6 Hz, 1H), 8.76 (s, 1H), 8.59 (s, 1H), 8.56 (s, 1H), 8.40 (d, J=6.8 Hz, 2H), 8.18 (d, J=3.6 Hz, 1H), 4.70-4.62 (m, 1H), 3.31 (d, J=12.7 Hz, 2H), 3.17 (d, J=5.6 Hz, 1H), 2.85 (s, 1H), 2.09-1.75 (m, 4H), 1.50 (d, J=6.6 Hz, 6H).

LC/MS: [M+H] for C₁₉H₂₄N₈O calculated. 380.21 found. 381.2.

HPLC: 94% pure (254 nM) at 3:81 minutes.

### Example 79: 6-(1,3-dimethyl-1H-pyrazol-4-yl)-4-{[(3S)-piperidin-3-yl]amino}pyrido[3,2-d]pyrimidine-8-carboxamide

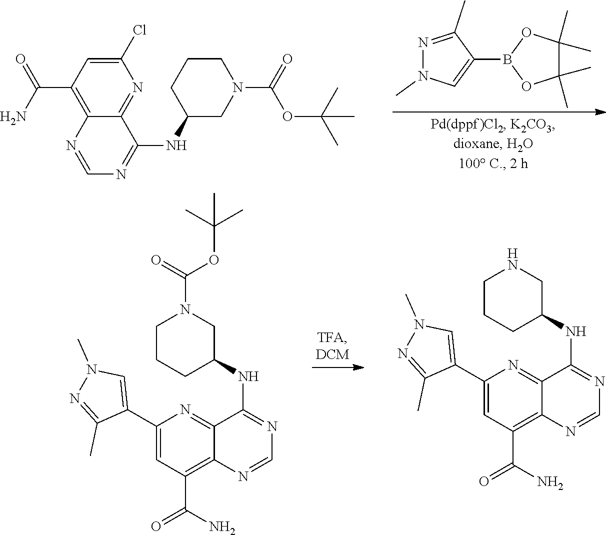

tert-butyl (3S)-3-{[8-carbamoyl-6-(1,3-dimethyl-1H-pyrazol-4-yl)pyrido[3,2-d]pyrimidin-4-yl]amino}piperidine-1-carboxylate: tert-butyl (3S)-3-({8-carbamoyl-6-chloropyrido[3,2-d]pyrimidin-4-yl}amino)piperidine-1-carboxylate (50.0 mg; 0.123 mmol; 1.00 eq.), 1,3-dimethyl-4-(4,4,5,5-tetramethyl-1,3,2-dioxaborolan-2-yl)-1h-pyrazole (40.9 mg; 0.18 mmol; 1.50 eq.), potassium carbonate (51.0 mg; 0.369 mmol; 3.00 eq.), Pd(dppf)Cl₂ (5.0 mg; 0.006 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h. The reaction mixture was filtered through celite, concentrated under reduced pressure, to give tert-butyl (3S)-3-{[8-carbamoyl-6-(1,3-dimethyl-1H-pyrazol-4-yl)pyrido[3,2-d]pyrimidin-4-yl]amino}piperidine-1-carboxylate (40.0 mg; 0.09 mmol; 69.8%) crude.

6-(1,3-dimethyl-1H-pyrazol-4-yl)-4-{[(3S)-piperidin-3-yl]amino}pyrido[3,2-d]pyrimidine-8-carboxamide: tert-butyl (3S)-3-{[8-carbamoyl-6-(1,3-dimethyl-1H-pyrazol-4-yl)pyrido[3,2-d]pyrimidin-4-yl]amino}piperidine-1-carboxylate (40.0 mg; 0.09 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The sample was purified on an Agilent 1100 with an Aurora Fusion A5 SFC (CEL4, 4.6×150 mm, particle size of 5 um, pore size 1000 A) with a column temperature of 40 C and mobile phases A: CO₂ B: Methanol+20 mM NH₄OH. Separation was achieved using a gradient of 85% solvent A+15% solvent B to 55% solvent A+45% solvent B. The desired fractions were combined and dried down via TurboVap using nitrogen flow to give 6-(1,3-dimethyl-1H-pyrazol-4-yl)-4-{[(3S)-piperidin-3-yl]amino}pyrido[3,2-d]pyrimidine-8-carboxamide (14.0 mg; 0.04 mmol; 44.6%) as a yellow solid.

¹H NMR (400 MHz, DMSO) δ 10.03 (d, J=3.7 Hz, 1H), 8.58 (s, 1H), 8.53 (d, J=2.1 Hz, 2H), 8.17 (t, J=4.7 Hz, 1H), 8.00 (d, J=8.5 Hz, 1H), 4.24 (td, J=7.4, 3.6 Hz, 1H), 3.84 (s, 3H), 3.17 (d, J=4.5 Hz, 1H), 2.99 (dd, J=11.7, 3.2 Hz, 1H), 2.73 (dt, J=17.1, 5.9 Hz, 3H), 2.56 (s, 3H), 1.87-1.73 (m, 2H), 1.65 (dd, J=10.8, 6.1 Hz, 1H), 1.48 (dq, J=13.0, 5.9 Hz, 1H).

LC/MS: [M+H] for C₁₈H₂₂N₈O calculated. 366.19 found. 367.2.

HPLC: 100% pure (254 nM) at 1:69 minutes.

### Example 80: 6-{[(3Z)-oxolan-3-ylidene]methyl}-4-{[(3S)-piperidin-3-yl]amino}pyrido[3,2-d]pyrimidine-8-carboxamide

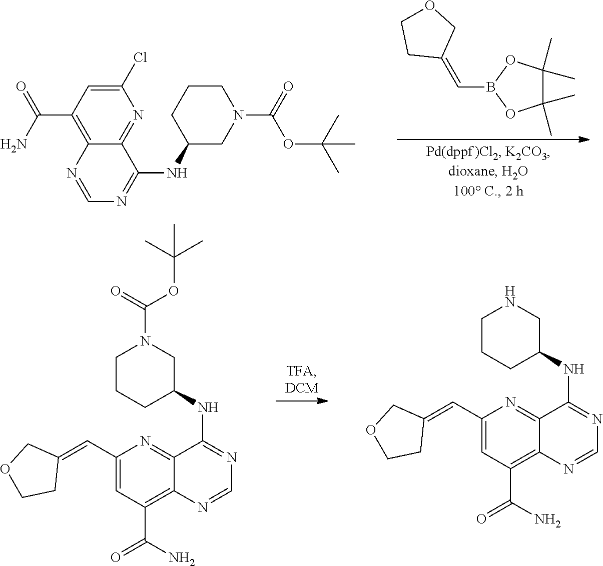

tert-butyl (3S)-3-[(8-carbamoyl-6-{[(3Z)-oxolan-3-ylidene]methyl}pyrido[3,2-d]pyrimidin-4-yl)amino]piperidine-1-carboxylate: tert-butyl (3S)-3-({8-carbamoyl-6-chloropyrido[3,2-d]pyrimidin-4-yl}amino)piperidine-1-carboxylate (80.0 mg; 0.20 mmol; 1.00 eq.), 4,4,5,5-tetramethyl-2-[(oxolan-3-ylidene)methyl]-1,3,2-dioxaborolane (61.9 mg; 0.29 mmol; 1.50 eq.), potassium carbonate (81.5 mg; 0.59 mmol; 3.00 eq.), Pd(dppf)Cl₂ (8.0 mg; 0.01 mmol; 0.05 eq.), Dioxane (1.6 ml) and water (0.2 ml) were added to a microwave vial. The tube was sealed and flushed with nitrogen for 5 min and the suspension was microwaved at 100° C. for 2 h. The reaction mixture was filtered through celite, concentrated under reduced pressure, to give tert-butyl (3S)-3-[(8-carbamoyl-6-{[(3Z)-oxolan-3-ylidene]methyl}pyrido[3,2-d]pyrimidin-4-yl)amino]piperidine-1-carboxylate (89.0 mg; 0.20 mmol; 99.6%) crude.

6-{[(3Z)-oxolan-3-ylidene]methyl}-4-{[(3S)-piperidin-3-yl]amino}pyrido[3,2-d]pyrimidine-8-carboxamide: tert-butyl (3S)-3-{[8-carbamoyl-6-(1,3-dimethyl-1H-pyrazol-4-yl)pyrido[3,2-d]pyrimidin-4-yl]amino}piperidine-1-carboxylate (29.0 mg; 0.06 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The sample was purified on an Agilent 1100 with an Aurora Fusion A5 SFC (WHELK-01, 4.6×150 mm, particle size of 5 um, pore size 1000 A) with a column temperature of 40 C and mobile phases A: CO₂ B: Methanol+20 mM NH₄OH. Separation was achieved using a gradient of 85% solvent A+15% solvent B to 55% solvent A+45% solvent B. The desired fractions were combined and dried down via TurboVap using nitrogen flow to give 6-{[(3Z)-oxolan-3-ylidene]methyl}-4-{[(3S)-piperidin-3-yl]amino}pyrido[3,2-d]pyrimidine-8-carboxamide (14.0 mg; 0.04 mmol; 58.8%) as a yellow solid.

¹H NMR (400 MHz, DMSO) δ 9.99 (s, 1H), 8.55 (s, 1H), 8.29 (s, 1H), 8.15 (d, J=3.7 Hz, 1H), 7.84 (d, J=8.5 Hz, 1H), 6.88 (p, J=2.3 Hz, 1H), 4.91-4.75 (m, 2H), 4.27 (s, 1H), 3.84 (t, J=6.8 Hz, 2H), 2.98 (d, J=10.8 Hz, 1H), 2.87-2.70 (m, 5H), 1.83-1.76 (m, 2H), 1.73-1.43 (m, 3H).

LC/MS: [M+H] for C₁₈H₂₂N₆O₂ calculated. 354.18 found. 355.2.

HPLC: 100% pure (254 nM) at 1:66 minutes.

### Example 81: 6-[1-(2,2-difluoroethyl)-1H-pyrazol-4-yl]-4-{[(3S)-piperidin-3-yl]amino}pyrido[3,2-d]pyrimidine-8-carboxamide

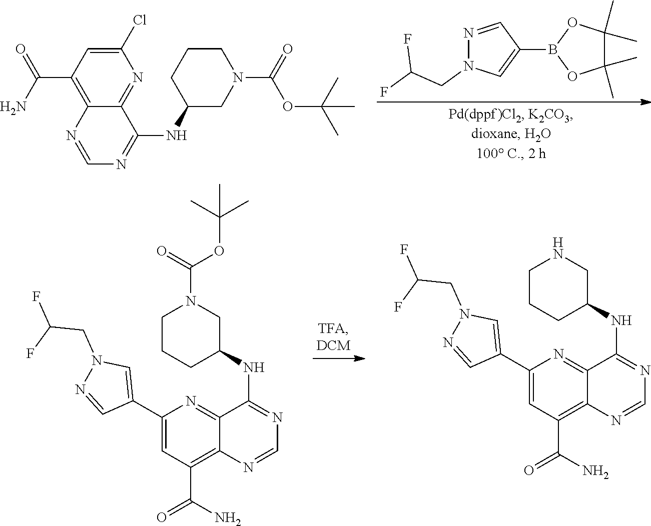

tert-butyl (3S)-3-[(8-carbamoyl-6-{[(3Z)-oxolan-3-ylidene]methyl}pyrido[3,2-d]pyrimidin-4-yl)amino]piperidine-1-carboxylate: tert-butyl (3S)-3-({8-carbamoyl-6-chloropyrido[3,2-d]pyrimidin-4-yl}amino)piperidine-1-carboxylate (50.0 mg; 0.12 mmol; 1.00 eq.), 1-(2,2-difluoroethyl)-4-(4,4,5,5-tetramethyl-1,3,2-dioxaborolan-2-yl)-1h-pyrazole (47.57 mg; 0.18 mmol; 1.50 eq.), potassium carbonate (50.9 mg; 0.37 mmol; 3.00 eq.), Pd(dppf)Cl₂ (5.0 mg; 0.01 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h. The reaction mixture was filtered through celite, concentrated under reduced pressure, to give tert-butyl (3S)-3-[(8-carbamoyl-6-{[(3Z)-oxolan-3-ylidene]methyl}pyrido[3,2-d]pyrimidin-4-yl)amino]piperidine-1-carboxylate (89.0 mg; 0.20 mmol; 99.6%) crude.

6-[1-(2,2-difluoroethyl)-1H-pyrazol-4-yl]-4-{[(3S)-piperidin-3-yl]amino}pyrido[3,2-d]pyrimidine-8-carboxamide: tert-butyl (3S)-3-({8-carbamoyl-6-[1-(2,2-difluoroethyl)-1H-pyrazol-4-yl]pyrido[3,2-d]pyrimidin-4-yl}amino)piperidine-1-carboxylate (61.0 mg; 0.12 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The sample was purified on an Agilent 1100 with an Aurora Fusion A5 SFC (CEL4, 4.6×150 mm, particle size of 5 um, pore size 1000 A) with a column temperature of 40 C and mobile phases A: CO₂ B: Methanol+20 mM NH₄OH. Separation was achieved using a gradient of 85% solvent A+15% solvent B to 55% solvent A+45% solvent B. The desired fractions were combined and dried down via TurboVap using nitrogen flow to give 6-[1-(2,2-difluoroethyl)-1H-pyrazol-4-yl]-4-{[(3S)-piperidin-3-yl]amino}pyrido[3,2-d]pyrimidine-8-carboxamide (23.9 mg; 0.06 mmol; 49.8%) as a yellow solid.

¹H NMR (400 MHz, DMSO) δ 10.01 (d, J=3.7 Hz, 1H), 8.76 (s, 1H), 8.56 (s, 1H), 8.52 (s, 1H), 8.49 (s, 1H), 8.17 (d, J=8.4 Hz, 2H), 6.46 (tt, J=54.8, 3.7 Hz, 1H), 4.72 (td, J=15.1, 3.8 Hz, 2H), 4.30-4.20 (m, 1H), 3.06 (dd, J=11.8, 3.8 Hz, 1H), 2.83 (dt, J=12.5, 4.1 Hz, 1H), 2.67 (dd, J=11.7, 9.0 Hz, 1H), 2.55 (dd, J=10.3, 3.0 Hz, 1H), 1.94 (dd, J=12.7, 4.7 Hz, 1H), 1.81-1.64 (m, 2H), 1.49 (qd, J=9.3, 5.3 Hz, 1H).

LC/MS: [M+H] for C₁₈H₂₀F₂N₈O calculated. 402.17 found. 402.8.

HPLC: 100% pure (254 nM) at 1:56 minutes.

### Example 82: 6-[4-(3-hydroxyoxetan-3-yl)phenyl]-4-{[(3S)-piperidin-3-yl]amino}pyrido[3,2-d]pyrimidine-8-carboxamide

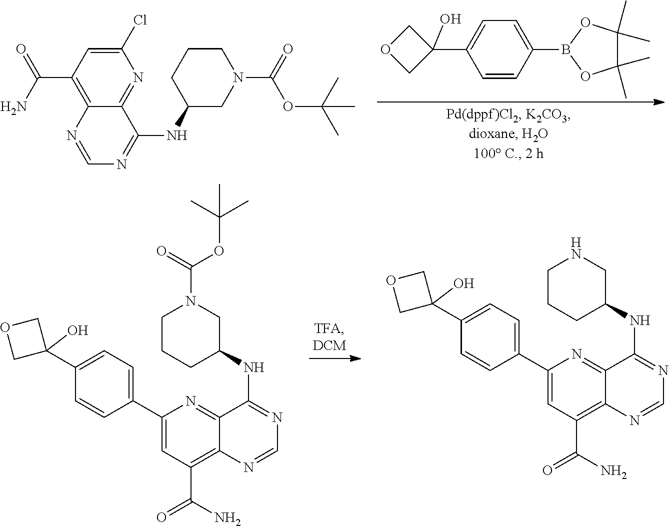 [SMILES: CC(C)(C)OC(=O)N1CCC[C@H](Nc2ncnc3c(C(N)=O)cc(Cl)nc23)C1 ; CC1(C)OB(c2ccc(C3(O)COC3)cc2)OC1(C)C ; CC(C)(C)OC(=O)N1CCC[C@H](Nc2ncnc3c(C(N)=O)cc(-c4ccc(C5(O)COC5)cc4)nc23)C1 ; NC(=O)c1cc(-c2ccc(C3(O)COC3)cc2)nc2c(N[C@H]3CCCNC3)ncnc12]

tert-butyl (3S)-3-{[8-carbamoyl-6-(1H-pyrazol-4-yl)pyrido[3,2-d]pyrimidin-4-yl]amino}piperidine-1-carboxylate: tert-butyl (3S)-3-({8-carbamoyl-6-chloropyrido[3,2-d]pyrimidin-4-yl}amino)piperidine-1-carboxylate (50.0 mg; 0.12 mmol; 1.00 eq.), 3-[4-(4,4,5,5-tetramethyl-1,3,2-dioxaborolan-2-yl)phenyl]-3-oxetanol (50.9 mg; 0.18 mmol; 1.50 eq.), potassium carbonate (50.95 mg; 0.37 mmol; 3.00 eq.), Pd(dppf)Cl₂ (5.0 mg; 0.01 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h. The reaction mixture was filtered through celite, concentrated under reduced pressure, to give tert-butyl (3S)-3-{[8-carbamoyl-6-(1H-pyrazol-4-yl)pyrido[3,2-d]pyrimidin-4-yl]amino}piperidine-1-carboxylate (64.0 mg; 0.12 mmol; 100%) crude.

6-[4-(3-hydroxyoxetan-3-yl)phenyl]-4-{[(3S)-piperidin-3-yl]amino}pyrido[3,2-d]pyrimidine-8-carboxamide: tert-butyl (3S)-3-{[8-carbamoyl-6-(1H-pyrazol-4-yl)pyrido[3,2-d]pyrimidin-4-yl]amino}piperidine-1-carboxylate (64.0 mg; 0.12 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The product was purified on waters reverse phase system using a gradient of 10-60% CH₃CN/H₂O (0.1% Ammonium Hydroxide) in 3 injections. The desired fractions were combined and dried down via TurboVap using nitrogen flow to give 6-[4-(3-hydroxyoxetan-3-yl)phenyl]-4-{[(3S)-piperidin-3-yl]amino}pyrido[3,2-d]pyrimidine-8-carboxamide (16 mg; 0.04 mmol; 30.3%) as a yellow solid.

¹H NMR (400 MHz, DMSO) δ 10.05 (d, J=3.7 Hz, 1H), 8.87 (s, 1H), 8.58 (s, 1H), 8.39 (t, J=8.4 Hz, 3H), 8.23 (d, J=3.7 Hz, 1H), 7.82 (d, J=8.5 Hz, 2H), 6.51 (s, 1H), 4.80 (dd, J=37.7, 6.5 Hz, 4H), 4.30 (qt, J=8.4, 3.9 Hz, 1H), 3.05 (dd, J=11.9, 3.7 Hz, 1H), 2.78 (ddd, J=27.9, 11.9, 8.1 Hz, 2H), 2.58 (ddd, J=12.3, 9.4, 3.1 Hz, 1H), 1.96-1.78 (m, 2H), 1.69 (dt, J=13.6, 4.4 Hz, 1H), 1.50 (dp, J=13.5, 5.2 Hz, 1H).

LC/MS: [M+H] for C₂₂H₂₄N₆O₃ calculated. 420.19 found. 421.2.

HPLC: 98% pure (254 nM) at 1:62 minutes.

### Example 83: 6-(5-cyano-1-methyl-1H-pyrrol-3-yl)-4-{[(3S)-piperidin-3-yl]amino}pyrido[3,2-d]pyrimidine-8-carboxamide

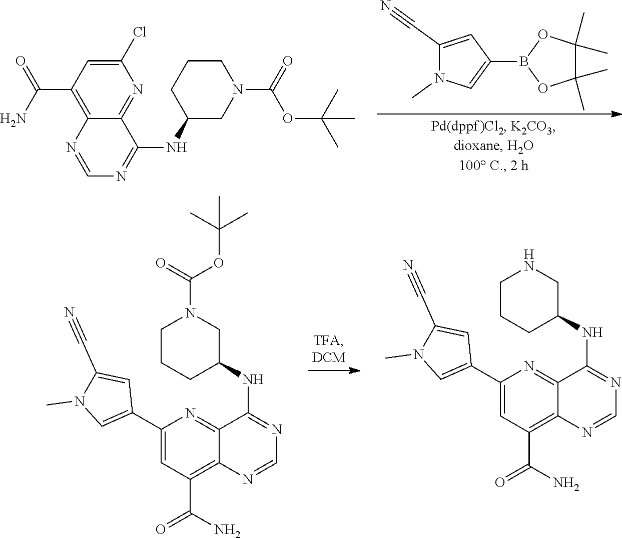 [SMILES: CC(C)(C)OC(=O)N1CCC[C@H](Nc2ncnc3c(C(N)=O)cc(Cl)nc23)C1 ; Cn1cc(B2OC(C)(C)C(C)(C)O2)cc1C#N ; Cn1cc(-c2cc(C(N)=O)c3ncnc(N[C@H]4CCCN(C(=O)OC(C)(C)C)C4)c3n2)cc1C#N ; Cn1cc(-c2cc(C(N)=O)c3ncnc(N[C@H]4CCCNC4)c3n2)cc1C#N]

tert-butyl (3S)-3-{[8-carbamoyl-6-(5-cyano-1-methyl-1H-pyrrol-3-yl)pyrido[3,2-d]pyrimidin-4-yl]amino}piperidine-1-carboxylate: tert-butyl (3S)-3-({8-carbamoyl-6-chloropyrido[3,2-d]pyrimidin-4-yl}amino)piperidine-1-carboxylate (50.0 mg; 0.12 mmol; 1.00 eq.), 1-methyl-4-(4,4,5,5-tetramethyl-1,3,2-dioxaborolan-2-yl)-1h-pyrrole-2-carbonitrile (42.78 mg; 0.18 mmol; 1.50 eq.), potassium carbonate (50.95 mg; 0.37 mmol; 3.00 eq.), Pd(dppf)Cl₂ iron (5.0 mg; 0.01 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h. The reaction mixture was filtered through celite, concentrated under reduced pressure, to give tert-butyl (3S)-3-{[8-carbamoyl-6-(5-cyano-1-methyl-1H-pyrrol-3-yl)pyrido[3,2-d]pyrimidin-4-yl]amino}piperidine-1-carboxylate (58.0 mg; 0.12 mmol; 100%) crude.

6-(5-cyano-1-methyl-1H-pyrrol-3-yl)-4-{[(3S)-piperidin-3-yl]amino}pyrido[3,2-d]pyrimidine-8-carboxamide: tert-butyl (3S)-3-{[8-carbamoyl-6-(5-cyano-1-methyl-1H-pyrrol-3-yl)pyrido[3,2-d]pyrimidin-4-yl]amino}piperidine-1-carboxylate (58.0 mg; 0.12 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The sample was purified on an Agilent 1100 with an Aurora Fusion A5 SFC (WHELK-01, 4.6×150 mm, particle size of Sum, pore size 1000 A) with a column temperature of 40 C and mobile phases A: CO₂ B: Methanol+20 mM NH₄OH. Separation was achieved using a gradient of 85% solvent A+15% solvent B to 55% solvent A+45% solvent B. The desired fractions were combined and dried down via TurboVap using nitrogen flow to give 6-(5-cyano-1-methyl-1H-pyrrol-3-yl)-4-{[(3S)-piperidin-3-yl]amino}pyrido[3,2-d]pyrimidine-8-carboxamide (19 mg; 0.05 mmol; 41.1%) as a yellow solid.

¹H NMR (400 MHz, DMSO) δ 10.01 (d, J=3.7 Hz, 1H), 8.55 (s, 1H), 8.51 (s, 1H), 8.25 (d, J=1.8 Hz, 1H), 8.17 (d, J=8.6 Hz, 2H), 7.95 (d, J=1.8 Hz, 1H), 4.26 (d, J=9.6 Hz, 1H), 3.85 (s, 3H), 3.07 (d, J=10.5 Hz, 1H), 2.85 (d, J=12.2 Hz, 1H), 2.70-2.63 (m, 1H), 1.99-1.66 (m, 4H), 1.50 (d, J=12.4 Hz, 1H).

LC/MS: [M+H] for C₂₂H₂₄N₆O₃ calculated. 376.18 found. 377.2.

HPLC: 99% pure (254 nM) at 1:91 minutes.

### Example 84: 6-[(oxolan-3-yl)methyl]-4-{[(3S)-piperidin-3-yl]amino}pyrido[3,2-d]pyrimidine-8-carboxamide

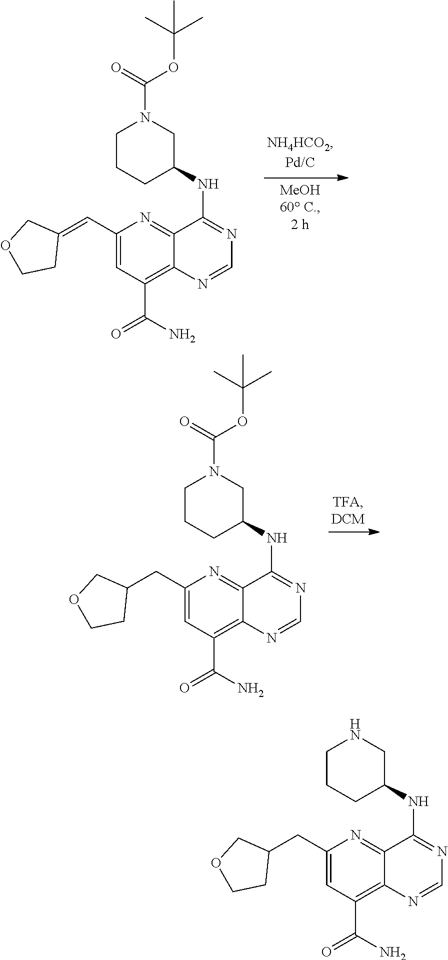 [SMILES: CC(C)(C)OC(=O)N1CCC[C@H](Nc2ncnc3c(C(N)=O)cc(/C=C4\CCOC4)nc23)C1 ; CC(C)(C)OC(=O)N1CCC[C@H](Nc2ncnc3c(C(N)=O)cc(CC4CCOC4)nc23)C1 ; NC(=O)c1cc(CC2CCOC2)nc2c(N[C@H]3CCCNC3)ncnc12]

tert-butyl (3S)-3-({8-carbamoyl-6-[(oxolan-3-yl)methyl]pyrido[3,2-d]pyrimidin-4-yl}amino)piperidine-1-carboxylate: In a microwave vial with a magnetic stir bar, tert-butyl (3S)-3-[(8-carbamoyl-6-{[(3Z)-oxolan-3-ylidene]methyl}pyrido[3,2-d]pyrimidin-4-yl)amino]piperidine-1-carboxylate (59.00 mg; 0.13 mmol; 1.00 eq.), ammonium formate (40.93 mg; 0.65 mmol; 5.00 eq.), methanol (2.00 ml) and palladium on carbon (20.00 mg) was added under argon. The resulting mixture was heated to 60° C. for 16 h. The reaction mixture was filtered through celite, concentrated under reduced pressure, to give tert-butyl (3S)-3-({8-carbamoyl-6-[(oxolan-3-yl)methyl]pyrido[3,2-d]pyrimidin-4-yl}amino)piperidine-1-carboxylate (35.0 mg; 0.08 mmol; 100%) crude.

6-[(oxolan-3-yl)methyl]-4-{[(3S)-piperidin-3-yl]amino}pyrido[3,2-d]pyrimidine-8-carboxamide: tert-butyl (3S)-3-({8-carbamoyl-6-[(oxolan-3-yl)methyl]pyrido[3,2-d]pyrimidin-4-yl}amino)piperidine-1-carboxylate (35.0 mg; 0.08 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The product was purified on waters reverse phase system using a gradient of 10-60% CH₃CN/H₂O (0.1% Ammonium Hydroxide) in 3 injections. The desired fractions were combined and dried down via TurboVap using nitrogen flow to give 6-[(oxolan-3-yl)methyl]-4-{[(3S)-piperidin-3-yl]amino}pyrido[3,2-d]pyrimidine-8-carboxamide (13 mg; 0.04 mmol; 46.6%) as a yellow solid.

¹H NMR (400 MHz, DMSO) δ 10.02 (d, J=3.7 Hz, 1H), 8.54 (s, 1H), 8.25 (s, 1H), 8.15 (d, J=8.5 Hz, 2H), 4.24 (qt, J=7.9, 3.7 Hz, 1H), 3.84-3.75 (m, 2H), 3.66 (q, J=7.6 Hz, 1H), 3.42 (ddd, J=8.4, 6.6, 3.8 Hz, 1H), 3.07 (d, J=7.5 Hz, 2H), 2.98 (dd, J=11.6, 3.4 Hz, 1H), 2.73 (ddd, J=19.1, 13.3, 7.4 Hz, 3H), 2.61 (ddd, J=11.9, 8.3, 3.4 Hz, 1H), 1.98 (dtt, J=12.5, 7.8, 4.9 Hz, 1H), 1.85-1.72 (m, 2H), 1.62 (ddp, J=11.2, 7.8, 3.5 Hz, 2H), 1.46 (dtt, J=12.8, 8.4, 4.0 Hz, 1H).

LC/MS: [M+H] for C₂₂H₂₄N₆O₃ calculated. 356.2 found. 356.9.

HPLC: 98% pure (254 nM) at 1:58 minutes.

### Example 85: 6-[(oxan-4-ylidene)methyl]-4-{[(3S)-piperidin-3-yl]amino}pyrido[3,2-d]pyrimidine-8-carboxamide

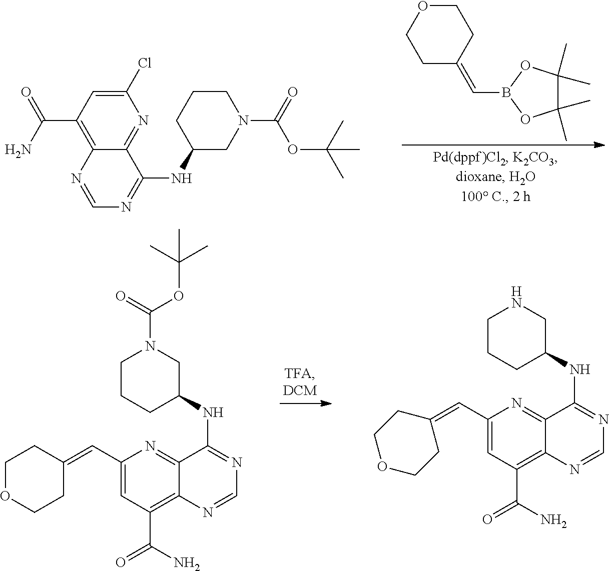

tert-butyl (3S)-3-({8-carbamoyl-6-[(oxan-4-ylidene)methyl]pyrido[3,2-d]pyrimidin-4-yl}amino)piperidine-1-carboxylate: tert-butyl (3S)-3-({8-carbamoyl-6-chloropyrido[3,2-d]pyrimidin-4-yl}amino)piperidine-1-carboxylate (80.0 mg; 0.20 mmol; 1.00 eq.), ((dihydro-2h-pyran-4 (3h)-ylidene)methyl)boronic acid pinacol ester (66.1 mg; 0.29 mmol; 1.50 eq.), potassium carbonate (81.5 mg; 0.59 mmol; 3.00 eq.), Pd(dppf)Cl₂ (8.0 mg; 0.01 mmol; 0.05 eq.), Dioxane (1.6 ml) and water (0.2 ml) were added to a microwave vial. The tube was sealed and flushed with nitrogen for 5 min and the suspension was microwaved at 100° C. for 2 h. The reaction mixture was filtered through celite, concentrated under reduced pressure, to give tert-butyl (3S)-3-({8-carbamoyl-6-[(oxan-4-ylidene)methyl]pyrido[3,2-d]pyrimidin-4-yl}amino)piperidine-1-carboxylate (92.0 mg; 0.20 mmol; 99.9%) crude.

6-[(oxan-4-ylidene)methyl]-4-{[(3S)-piperidin-3-yl]amino}pyrido[3,2-d]pyrimidine-8-carboxamide: tert-butyl (3S)-3-({8-carbamoyl-6-[(oxan-4-ylidene)methyl]pyrido[3,2-d]pyrimidin-4-yl}amino)piperidine-1-carboxylate (30.0 mg; 0.06 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The sample was purified on an Agilent 1100 with an Aurora Fusion A5 SFC (WHELK-01, 4.6×150 mm, particle size of Sum, pore size 1000 A) with a column temperature of 40 C and mobile phases A: CO₂ B: Methanol+20 mM NH₄OH. Separation was achieved using a gradient of 85% solvent A+15% solvent B to 55% solvent A+45% solvent B. The desired fractions were combined and dried down via TurboVap using nitrogen flow to give 6-[(oxan-4-ylidene)methyl]-4-{[(3S)-piperidin-3-yl]amino}pyrido[3,2-d]pyrimidine-8-carboxamide (15.0 mg; 0.04 mmol; 62.7%) as a yellow solid.

¹H NMR (400 MHz, DMSO) δ 9.98 (d, J=3.6 Hz, 1H), 8.54 (s, 1H), 8.23 (s, 1H), 8.17-8.08 (m, 2H), 6.63 (t, J=1.3 Hz, 1H), 4.30-4.22 (m, 1H), 3.71 (dt, J=25.7, 5.3 Hz, 4H), 3.19-3.15 (m, 1H), 3.00-2.92 (m, 3H), 2.79-2.69 (m, 3H), 2.44 (t, J=5.4 Hz, 2H), 1.78 (q, J=6.0 Hz, 2H), 1.61 (dt, J=12.7, 6.3 Hz, 1H), 1.52-1.43 (m, 1H).

LC/MS: [M+H] for C₁₈H₂₂N₆O₂ calculated. 368.44 found. 369.2.

HPLC: 98% pure (254 nM) at 1:90 minutes.

### Example 86: 6-(2-cyanophenyl)-4-{[(3S)-piperidin-3-yl]amino}pyrido[3,2-d]pyrimidine-8-carboxamide

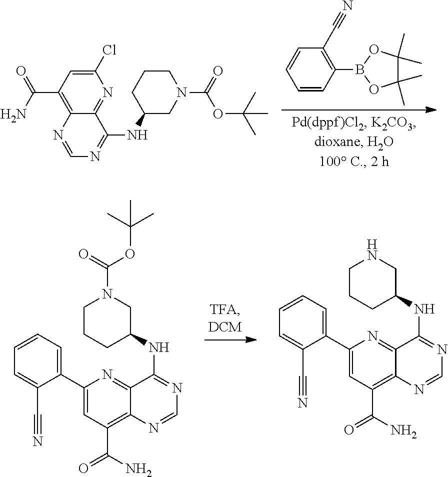 [SMILES: CC(C)(C)OC(=O)N1CCC[C@H](Nc2ncnc3c(C(N)=O)cc(Cl)nc23)C1 ; CC1(C)OB(c2ccccc2C#N)OC1(C)C ; CC(C)(C)OC(=O)N1CCC[C@H](Nc2ncnc3c(C(N)=O)cc(-c4ccccc4C#N)nc23)C1 ; N#Cc1ccccc1-c1cc(C(N)=O)c2ncnc(N[C@H]3CCCNC3)c2n1]

tert-butyl (3S)-3-{[8-carbamoyl-6-(2-cyanophenyl)pyrido[3,2-d]pyrimidin-4-yl]amino}piperidine-1-carboxylate: tert-butyl (3S)-3-({8-carbamoyl-6-chloropyrido[3,2-d]pyrimidin-4-yl}amino)piperidine-1-carboxylate (50.0 mg; 0.123 mmol; 1.00 eq.), 2-(4,4,5,5-tetramethyl-1,3,2-dioxaborolan-2-yl)benzonitrile (42.2 mg; 0.180 mmol; 1.50 eq.), potassium carbonate (51.0 mg; 0.369 mmol; 3.00 eq.), Pd(dppf)Cl₂ (5.0 mg; 0.006 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h. The reaction mixture was filtered through celite, concentrated under reduced pressure to give tert-butyl (3S)-3-{[8-carbamoyl-6-(2-cyanophenyl)pyrido[3,2-d]pyrimidin-4-yl]amino}piperidine-1-carboxylate (57.0 mg; 0.12 mmol; 99.7%) crude.

6-(2-cyanophenyl)-4-{[(3S)-piperidin-3-yl]amino}pyrido[3,2-d]pyrimidine-8-carboxamide: tert-butyl (3S)-3-{[8-carbamoyl-6-(2-cyanophenyl)pyrido[3,2-d]pyrimidin-4-yl]amino}piperidine-1-carboxylate (57.0 mg; 0.12 mmol; 1.00 eq.) was dissolved in dichloromethane (1.0 ml). FA (0.3 ml) was added to reaction mixture. The resulting solution was stirred for two hours. The volatiles were evaporated, and the residue dissolved in 3 mL of DMSO. The sample was purified on an Agilent 1100 with an Aurora Fusion A5 SFC (WHELK-01, 4.6×150 mm, particle size of Sum, pore size 1000 A) with a column temperature of 40 C and mobile phases A: CO₂ B: Methanol+20 mM NH₄OH. Separation was achieved using a gradient of 85% solvent A+15% solvent B to 55% solvent A+45% solvent B. The desired fractions were combined and dried down via TurboVap using nitrogen flow to give 6-(2-cyanophenyl)-4—{[(3S)-piperidin-3-yl]amino}pyrido[3,2-d]pyrimidine-8-carboxamide (12.0 mg; 0.03 mmol; 26.5%) as a yellow solid.

¹H NMR (400 MHz, DMSO) δ 9.95 (d, J=3.6 Hz, 1H), 8.86 (s, 1H), 8.65 (s, 1H), 8.30 (dd, J=16.3, 5.7 Hz, 2H), 8.08 (dd, J=7.8, 1.5 Hz, 2H), 7.91 (td, J=7.8, 1.4 Hz, 1H), 7.74 (td, J=7.6, 1.2 Hz, 1H), 4.28-4.21 (m, 1H), 3.13-3.07 (m, 1H), 2.80 (d, J=12.4 Hz, 1H), 2.64-2.55 (m, 2H), 1.95 (d, J=7.9 Hz, 1H), 1.75-1.61 (m, 3H), 1.48 (dd, J=9.0, 3.8 Hz, 1H).

LC/MS: [M+H] for C₁₈H₂₂N₆O₂ calculated. 373.17 found. 374.2.

HPLC: 95% pure (254 nM) at 2:36 minutes.

### Example 87: 4-{[(3S)-piperidin-3-yl]amino}-6-[2-(trifluoromethyl)pyridin-3-yl]pyrido[3,2-d]pyrimidine-8-carboxamide

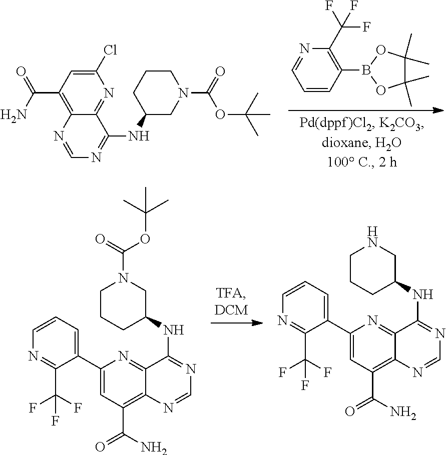 [SMILES: CC(C)(C)OC(=O)N1CCC[C@H](Nc2ncnc3c(C(N)=O)cc(Cl)nc23)C1 ; CC1(C)OB(c2cccnc2C(F)(F)F)OC1(C)C ; CC(C)(C)OC(=O)N1CCC[C@H](Nc2ncnc3c(C(N)=O)cc(-c4cccnc4C(F)(F)F)nc23)C1 ; NC(=O)c1cc(-c2cccnc2C(F)(F)F)nc2c(N[C@H]3CCCNC3)ncnc12]

tert-butyl (3S)-3-({8-carbamoyl-6-[2-(trifluoromethyl)pyridin-3-yl]pyrido[3,2-d]pyrimidin-4-yl}amino)piperidine-1-carboxylate: tert-butyl (3S)-3-({8-carbamoyl-6-chloropyrido[3,2-d]pyrimidin-4-yl}amino)piperidine-1-carboxylate (50.0 mg; 0.123 mmol; 1.00 eq.), 3-(4,4,5,5-tetramethyl-1,3,2-dioxaborolan-2-yl)-2-(trifluoromethyl)pyridine (41.5 mg; 0.110 mmol; 1.24 eq.), potassium carbonate (51.0 mg; 0.369 mmol; 3.00 eq.), Pd(dppf)Cl₂ (5.0 mg; 0.006 mmol; 0.05 eq.), Dioxane (1.0 ml) and water (0.2 ml) were added to a microwave vial. The tube was sealed and flushed with nitrogen for 5 min and the suspension was microwaved at 100° C. for 2 h. The reaction mixture was filtered through celite, concentrated under reduced pressure to give tert-butyl (3S)-3-({8-carbamoyl-6-[2-(trifluoromethyl)pyridin-3-yl]pyrido[3,2-d]pyrimidin-4-yl}amino)piperidine-1-carboxylate (50.0 mg; 0.10 mmol; 78.6%) crude.

4-{[(3S)-piperidin-3-yl]amino}-6-[2-(trifluoromethyl)pyridin-3-yl]pyrido[3,2-d]pyrimidine-8-carboxamide: tert-butyl (3S)-3-({8-carbamoyl-6-[2-(trifluoromethyl)pyridin-3-yl]pyrido[3,2-d]pyrimidin-4-yl}amino)piperidine-1-carboxylate (50.0 mg; 0.10 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The sample was purified on an Agilent 1100 with an Aurora Fusion A5 SFC (CEL4, 4.6×150 mm, particle size of 5 um, pore size 1000 A) with a column temperature of 40 C and mobile phases A: CO₂ B: Methanol+20 mM NH₄OH. Separation was achieved using a gradient of 85% solvent A+15% solvent B to 55% solvent A+45% solvent B. The desired fractions were combined and dried down via TurboVap using nitrogen flow to give 4-{[(3S)-piperidin-3-yl]amino}-6-[2-(trifluoromethyl)pyridin-3-yl]pyrido[3,2-d]pyrimidine-8-carboxamide (11.0 mg; 0.02 mmol; 25.1%) as a yellow solid.

¹H NMR (400 MHz, DMSO) δ 10.01 (d, J=3.6 Hz, 1H), 8.90 (dd, J=4.7, 1.5 Hz, 1H), 8.66 (s, 1H), 8.52 (s, 1H), 8.33-8.22 (m, 3H), 7.91 (dd, J=7.9, 4.7 Hz, 1H), 4.29 (tq, J=7.7, 3.9 Hz, 1H), 3.17 (s, 1H), 2.99 (dd, J=11.7, 3.4 Hz, 1H), 2.77-2.67 (m, 2H), 2.59 (ddd, J=11.7, 8.2, 3.4 Hz, 1H), 1.85-1.69 (m, 3H), 1.63 (ddt, J=13.4, 6.7, 3.5 Hz, 1H), 1.47 (dh, J=12.7, 4.1 Hz, 1H).

LC/MS: [M+H] for C₁₈H₂₂N₆O₂ calculated. 471.40 found. 417.8.

HPLC: 92% pure (254 nM) at 2:15 minutes.

### Example 88: 6-[(oxan-4-yl)methyl]-4-{[(3S)-piperidin-3-yl]amino}pyrido[3,2-d]pyrimidine-8-carboxamide

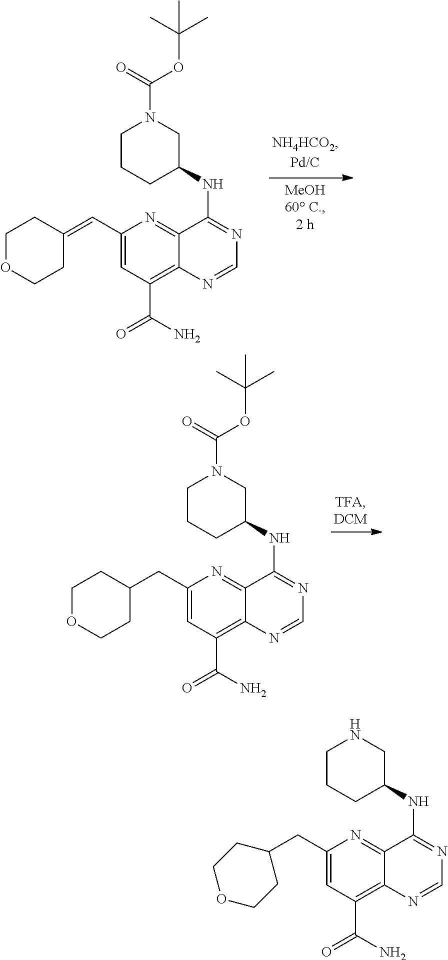 [SMILES: CC(C)(C)OC(=O)N1CCC[C@H](Nc2ncnc3c(C(N)=O)cc(C=C4CCOCC4)nc23)C1 ; CC(C)(C)OC(=O)N1CCC[C@H](Nc2ncnc3c(C(N)=O)cc(CC4CCOCC4)nc23)C1 ; NC(=O)c1cc(CC2CCOCC2)nc2c(N[C@H]3CCCNC3)ncnc12]

tert-butyl (3S)-3-({8-carbamoyl-6-[(oxan-4-yl)methyl]pyrido[3,2-d]pyrimidin-4-yl}amino)piperidine-1-carboxylate: In a microwave vial with a magnetic stir bar, tert-butyl (3S)-3-({8-carbamoyl-6-[(oxan-4-ylidene)methyl]pyrido[3,2-d]pyrimidin-4-yl}amino)piperidine-1-carboxylate (60.82 mg; 0.13 mmol; 1.00 eq.), ammonium formate (40.93 mg; 0.65 mmol; 5.00 eq.), methanol (2.00 ml) and palladium on carbon (20.00 mg) was added under argon. The resulting mixture was heated to 60*C for 16 h. The reaction mixture was filtered through celite, concentrated under reduced pressure, to give tert-butyl (3S)-3-({8-carbamoyl-6-[(oxan-4-yl)methyl]pyrido[3,2-d]pyrimidin-4-yl}amino)piperidine-1-carboxylate (61.0 mg; 0.13 mmol; 99.9%) crude.

6-[(oxan-4-yl)methyl]-4-{[(3S)-piperidin-3-yl]amino}pyrido[3,2-d]pyrimidine-8-carboxamide: tert-butyl (3S)-3-({8-carbamoyl-6-[(oxan-4-yl)methyl]pyrido[3,2-d]pyrimidin-4-yl}amino)piperidine-1-carboxylate (45.46 mg; 0.10 mmol; 1.00 eq.) was dissolved in dichloromethane (1.0 ml). TFA (0.3 ml) was added to reaction mixture. The resulting solution was stirred for two hours. The volatiles were evaporated, and the residue dissolved in 3 mL of DMSO. The product was purified on waters reverse phase system using a gradient of 10-60% CH₃CN/H₂O (0.1% Ammonium Hydroxide) in 3 injections. The desired fractions were combined and dried down via TurboVap using nitrogen flow to give 6-[(oxan-4-yl)methyl]-4-{[(3S)-piperidin-3-yl]amino}pyrido[3,2-d]pyrimidine-8-carboxamide (18.7 mg; 0.05 mmol; 52.2%) as a yellow solid.

¹H NMR (400 MHz, DMSO) δ 10.04 (d, J=3.8 Hz, 1H), 8.55 (s, 1H), 8.23 (s, 1H), 8.16 (dd, J=9.1, 6.1 Hz, 2H), 4.26 (tt, J=8.1, 4.1 Hz, 1H), 3.86-3.80 (m, 2H), 3.27 (d, J=11.4 Hz, 2H), 3.18 (s, 2H), 3.01 (dd, J=11.7, 3.5 Hz, 1H), 2.94 (d, J=7.2 Hz, 2H), 2.75 (td, J=12.3, 7.8 Hz, 2H), 2.62 (ddd, J=11.9, 8.4, 3.3 Hz, 1H), 2.09 (ddq, J=11.3, 7.7, 3.8 Hz, 1H), 1.79 (dtt, J=17.2, 8.6, 4.8 Hz, 2H), 1.65 (ddt, J=13.7, 7.0, 3.7 Hz, 1H), 1.55-1.45 (m, 3H), 1.38-1.26 (m, 2H).

LC/MS: [M+H] for C₂₂H₂₄N₆O₃ calculated. 370.2 found. 370.9.

HPLC: 100% pure (254 nM) at 1:72 minutes.

Examples with In Vitro and Cellular Activities.

### Table 1:

HPK1 kinase assay (Biochemical assay).

A recombinant fusion protein consisting of full-length human HPK1 (MAP4K₁) with an N-terminal Glutatione S-transferase (GST) tag was produced in insect cells Sf21 using the baculovirus expression system. GST-HPK1 protein was purified from cell lysates by glutathione Sepharose affinity chromatography. The assay is run in three continuous steps: 1) the HPK1 enzymatic kinase reaction, 2) an ATP depletion, and 3) the ADP detection, the steps 2 and 3 are performed with ADP-Glo™ Kinase Assay kit from Promega (V9101). Test compounds were prepared by 10-point serial dilution in dimethyl sulfoxide (DMSO) and 100 nL of each dilution was spotted onto a 384-well Optiplate (Perkin Elmer and Cat #6007299) by Labcyte Echo. 5 μL kinase reaction buffer (0.02% Brij-35, 2 mM DTT, 50 mM HEPES pH 7.5, MgCl2 10 mM, BSA 0.01% and β-glycerophosphate 12.5 mM) containing HPK1 (3.2 nM) enzyme was transferred to each well and incubated for 15 minutes at room temperature at 60% humidity. The enzymatic reaction was started by adding 5 μl of Start-Mix (10 μM ATP and 3.235 μM MBP). After 120 minutes, the reaction was stopped by adding 5 μL of ADP-Glo reagent (Promega, V9101) and incubating for 40 min. in the dark at 23° C. To determine the level of ADP, 10 μl ADP-Glo Detection solution was added and incubated for 1 hour at 23° C. in the dark. The plate was transferred to a Perkin Elmer EnVision (2104 Multilabel Reader) for luminescence detection and percent inhibition activity and IC₅₀ value were determined using Genedata Screener.

### Cellular Assay—Phospho-SLP-76 (Ser376).

Inhibition of HPK1 enzymatic activity in cells was determined by detection of SLP76 phosphorylation at Ser376 residue in lysate from Jurkat (human) and EL-4 (mouse) cells. Jurkat and EL-4 cells express both HPK1 and SLP76 naturally. All cells were cultured in RPMI 1640 media containing 10% heat inactivated fetal bovine serum (FBS) and 1× Penicillin-Streptomycin. In either Jurkat or El-4 assay, cells were pre-incubated with test compounds prior to T cell stimulation by respective αCD3/αCD28 Dynabeads™. For detection, plates were read on the Envision and percent inhibition and IC₅₀ were determined by Smart fit in Genedata Analyzer.

For Jurkat cells: 35 μL cell suspension were dispensed at a density of 30,000 cells/well in 384-well plates containing HBSS (Gibco, 14025076)+0.1% BSA). 10-point logarithmic serial dilution of test compounds in DMSO were prepared by a Hamilton automated liquid handling robot and 35 nL was transferred into each well by an Echo liquid handler. After 60 min. pre-incubation, the cells were stimulated by adding 5 μL human T activator αCD3/αCD28 Dynabeads™ (Gibco, 11132D) at 1.2×107 beads/ml for 15 minutes at 37° C. Detection of Ser376 phosphorylation status was accomplished by first adding 10 μL of 5× AlphaLISA lysis buffer (Perkin Elmer, ALSU-PSLP-A500) to each well of the cell plate using a Multidrop Combi (5840300), followed by 20 minutes shaking. 10 μL cell lysate from each treatment were then transferred to a white 384-well plate (Corning 3572) and incubated with AlphaLISA acceptor beads for one hour (AlphaLISA SureFire Ultra p-SLP76 (Ser376), Perkin Elmer, ALSU-PSLP-A500), and donor beads for additional 2 hours at room temperature in the dark.

For EL-4.IL2 assay, 50 μL cell suspension at 2×106 cells/mI in HBSS were first dispensed into 384-well plates and then 50 nL serial dilutions of test compounds were spotted to each well by an Echo liquid handler. After 60 min incubation at 37° C., cells were stimulated by adding 5 μL mouse T cell activator αCD3/αCD28 Dynabeads™ (ThermFisher Scientific, 11452D) for 30 minutes at 37° C. After a brief spin and subsequent supernatant discarded, 12.5 μL of Cisbio lysis buffer were added to each well and plates were shaken for 30 min at 450 RPM. For detection, 4 μL prepared HTRF antibody mixture (Cisbio, 63ADK000U) in detection buffer were mixed with 16 μl of cell lysate and plates were incubated at room temperature for 19 hours prior to EnVision reading.

### Results are Given in the Following Table.

### For Enzymatic Activity:

A: IC₅₀<100 nM
B: IC₅₀: 100 nM-1000 nM
C: IC₅₀>1000 nM

### For Cellular Activity:

A: IC₅₀<500 nM
B: IC₅₀: 500 nM-1000 nM
C: IC₅₀>1000 nM

| | | HPK1 | HPK1 |
|---|---|---|---|
| | | enzymatic | pSLP76 |
| # | Structure | IC₅₀ | IC₅₀ |
| Example 1 | 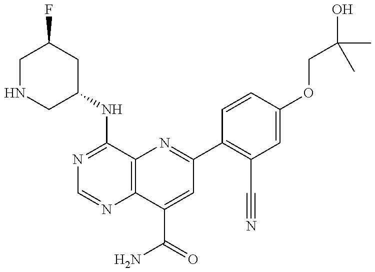 [SMILES: CC(C)(O)COc1ccc(-c2cc(C(N)=O)c3ncnc(N[C@@H]4CNC[C@@H](F)C4)c3n2)c(C#N)c1] | A | A |
| Example 2 | 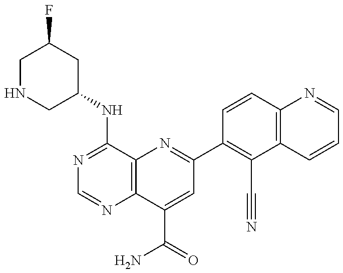 [SMILES: N#Cc1c(-c2cc(C(N)=O)c3ncnc(N[C@@H]4CNC[C@@H](F)C4)c3n2)ccc2ncccc12] | A | — |
| Example 3 | 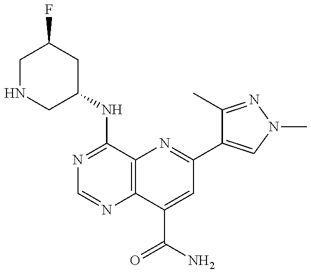 [SMILES: Cc1nn(C)cc1-c1cc(C(N)=O)c2ncnc(N[C@@H]3CNC[C@@H](F)C3)c2n1] | A | A |
| Example 4 | 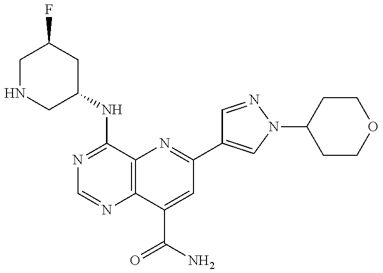 [SMILES: NC(=O)c1cc(-c2cnn(C3CCOCC3)c2)nc2c(N[C@@H]3CNC[C@@H](F)C3)ncnc12] | A | B |
| Example 5 | 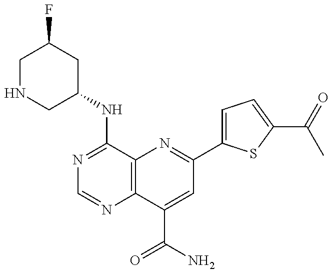 [SMILES: CC(=O)c1ccc(-c2cc(C(N)=O)c3ncnc(N[C@@H]4CNC[C@@H](F)C4)c3n2)s1] | A | A |
| Example 6 | 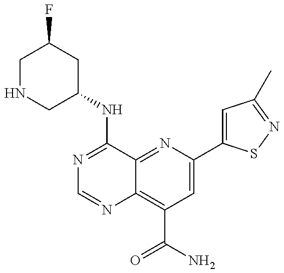 [SMILES: Cc1cc(-c2cc(C(N)=O)c3ncnc(N[C@@H]4CNC[C@@H](F)C4)c3n2)sn1] | A | — |
| Example 7 | 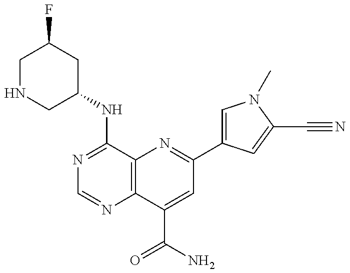 [SMILES: Cn1cc(-c2cc(C(N)=O)c3ncnc(N[C@@H]4CNC[C@@H](F)C4)c3n2)cc1C#N] | A | A |
| Example 8 | 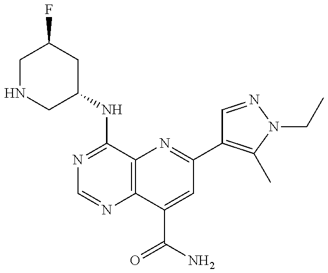 [SMILES: CCn1ncc(-c2cc(C(N)=O)c3ncnc(N[C@@H]4CNC[C@@H](F)C4)c3n2)c1C] | A | A |
| Example 9 | 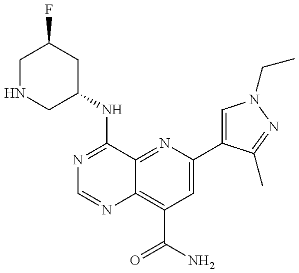 [SMILES: CCn1cc(-c2cc(C(N)=O)c3ncnc(N[C@@H]4CNC[C@@H](F)C4)c3n2)c(C)n1] | A | A |
| Example 10 | 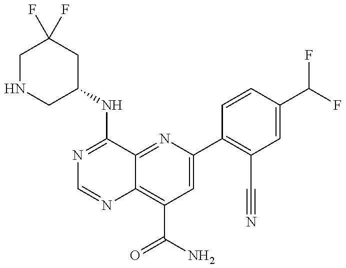 [SMILES: N#Cc1cc(C(F)F)ccc1-c1cc(C(N)=O)c2ncnc(N[C@@H]3CNCC(F)(F)C3)c2n1] | B | — |
| Example 11 | 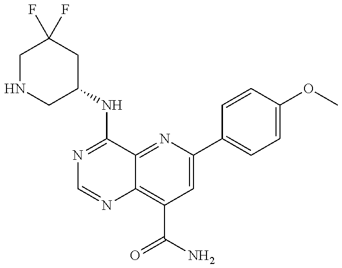 [SMILES: COc1ccc(-c2cc(C(N)=O)c3ncnc(N[C@@H]4CNCC(F)(F)C4)c3n2)cc1] | B | — |
| Example 12 | 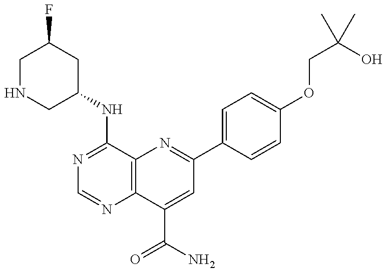 [SMILES: CC(C)(O)COc1ccc(-c2cc(C(N)=O)c3ncnc(N[C@@H]4CNC[C@@H](F)C4)c3n2)cc1] | A | A |
| Example 13 | 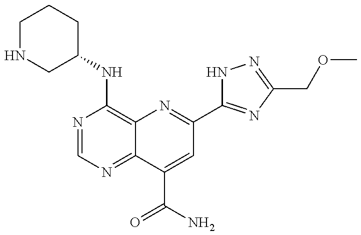 [SMILES: COCc1n[nH]c(-c2cc(C(N)=O)c3ncnc(N[C@H]4CCCNC4)c3n2)n1] | C | — |
| Example 14 | 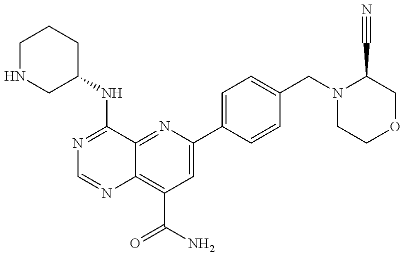 [SMILES: N#C[C@H]1COCCN1Cc1ccc(-c2cc(C(N)=O)c3ncnc(N[C@H]4CCCNC4)c3n2)cc1] | A | C |
| | Isomer 1 | | |
| | 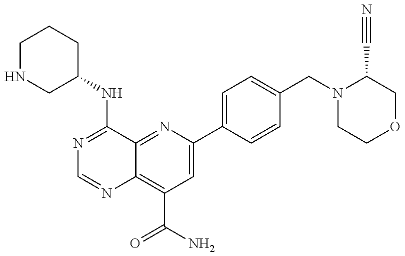 [SMILES: N#C[C@@H]1COCCN1Cc1ccc(-c2cc(C(N)=O)c3ncnc(N[C@H]4CCCNC4)c3n2)cc1] | | |
| | Isomer 2 | | |
| Example 15 | 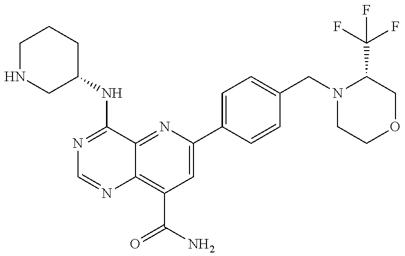 [SMILES: NC(=O)c1cc(-c2ccc(CN3CCOC[C@H]3C(F)(F)F)cc2)nc2c(N[C@H]3CCCNC3)ncnc12] | A | A |
| | Isomer 1 | | |
| | 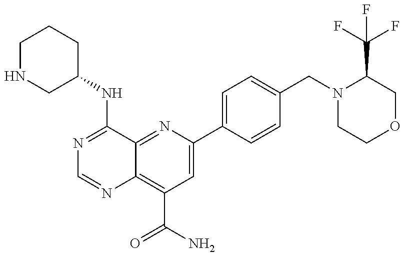 [SMILES: NC(=O)c1cc(-c2ccc(CN3CCOC[C@@H]3C(F)(F)F)cc2)nc2c(N[C@H]3CCCNC3)ncnc12] | | |
| | Isomer 2 | | |
| Example 16 | 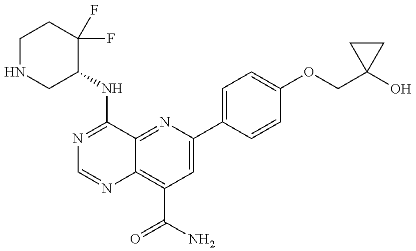 [SMILES: NC(=O)c1cc(-c2ccc(OCC3(O)CC3)cc2)nc2c(N[C@@H]3CNCCC3(F)F)ncnc12] | A | A |
| Example 17 | 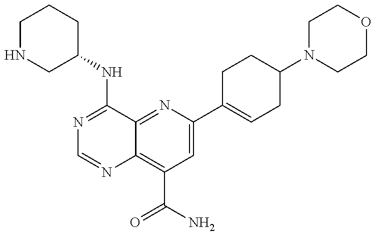 [SMILES: NC(=O)c1cc(C2=CCC(N3CCOCC3)CC2)nc2c(N[C@H]3CCCNC3)ncnc12] | A | B |
| Example 18 | 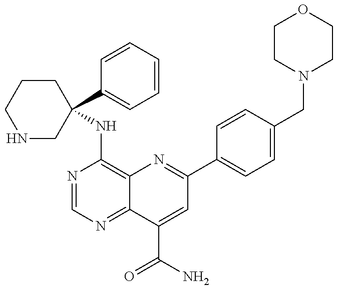 [SMILES: NC(=O)c1cc(-c2ccc(CN3CCOCC3)cc2)nc2c(N[C@@]3(c4ccccc4)CCCNC3)ncnc12] | B | C |
| | Isomer 1 | | |
| | 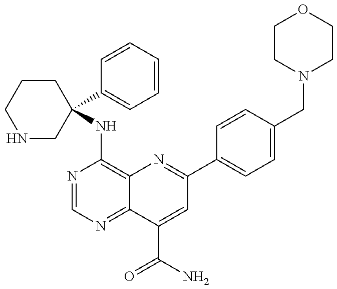 [SMILES: NC(=O)c1cc(-c2ccc(CN3CCOCC3)cc2)nc2c(N[C@]3(c4ccccc4)CCCNC3)ncnc12] | | |
| | Isomer 2 | | |
| Example 19 | 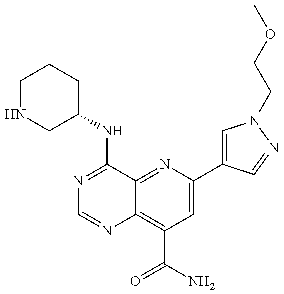 [SMILES: COCCn1cc(-c2cc(C(N)=O)c3ncnc(N[C@H]4CCCNC4)c3n2)cn1] | A | — |
| Example 20 | 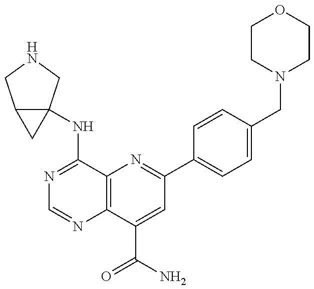 [SMILES: NC(=O)c1cc(-c2ccc(CN3CCOCC3)cc2)nc2c(NC34CNCC3C4)ncnc12] | A | C |
| | 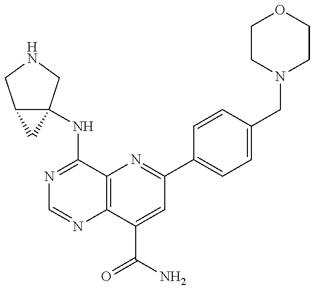 [SMILES: NC(=O)c1cc(-c2ccc(CN3CCOCC3)cc2)nc2c(N[C@]34CNC[C@H]3C4)ncnc12] | | |
| | Isomer 1 | | |
| | 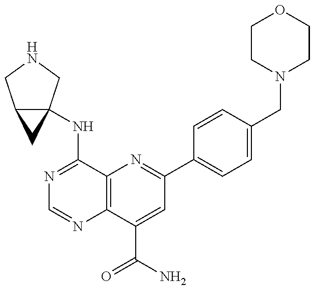 [SMILES: NC(=O)c1cc(-c2ccc(CN3CCOCC3)cc2)nc2c(N[C@@]34CNC[C@@H]3C4)ncnc12] | | |
| | Isomer 2 | | |
| Example 21 | 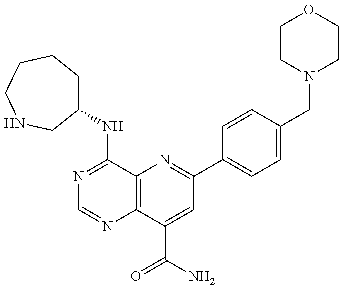 [SMILES: NC(=O)c1cc(-c2ccc(CN3CCOCC3)cc2)nc2c(N[C@H]3CCCCNC3)ncnc12] | B | — |
| | Isomer 1 | | |
| | 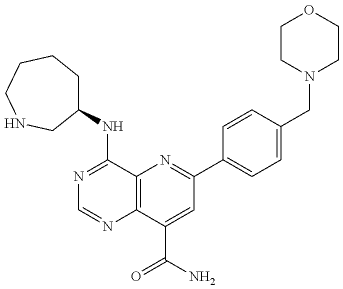 [SMILES: NC(=O)c1cc(-c2ccc(CN3CCOCC3)cc2)nc2c(N[C@@H]3CCCCNC3)ncnc12] | | |
| | Isomer 2 | | |
| Example 22 | 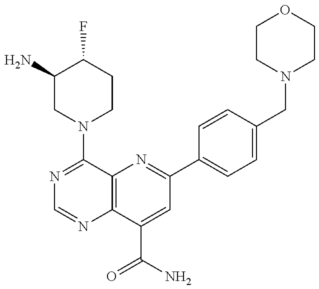 [SMILES: NC(=O)c1cc(-c2ccc(CN3CCOCC3)cc2)nc2c(N3CC[C@@H](F)[C@H](N)C3)ncnc12] | C | — |
| | Isomer 1 | | |
| | 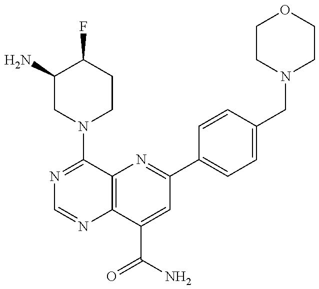 [SMILES: NC(=O)c1cc(-c2ccc(CN3CCOCC3)cc2)nc2c(N3CC[C@H](F)[C@H](N)C3)ncnc12] | | |
| | Isomer 2 | | |
| | 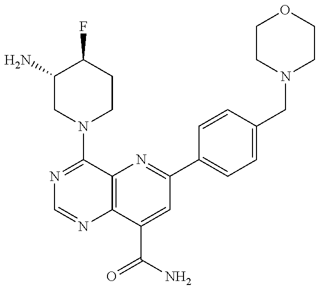 [SMILES: NC(=O)c1cc(-c2ccc(CN3CCOCC3)cc2)nc2c(N3CC[C@H](F)[C@@H](N)C3)ncnc12] | | |
| | Isomer 3 | | |
| | 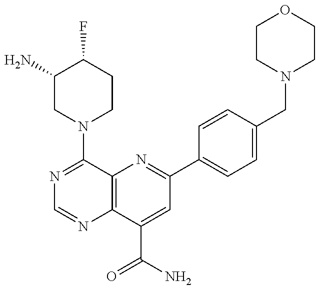 [SMILES: NC(=O)c1cc(-c2ccc(CN3CCOCC3)cc2)nc2c(N3CC[C@@H](F)[C@@H](N)C3)ncnc12] | | |
| | Isomer 4 | | |
| Example 23 | 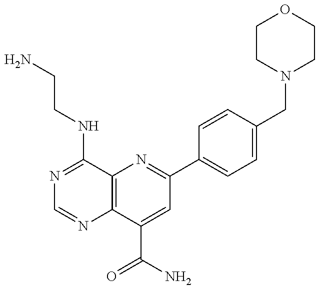 [SMILES: NCCNc1ncnc2c(C(N)=O)cc(-c3ccc(CN4CCOCC4)cc3)nc12] | A | — |
| Example 24 | 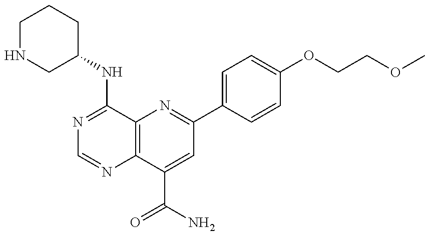 [SMILES: COCCOc1ccc(-c2cc(C(N)=O)c3ncnc(N[C@H]4CCCNC4)c3n2)cc1] | A | A |
| Example 25 | 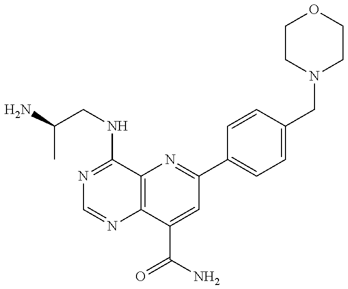 [SMILES: C[C@@H](N)CNc1ncnc2c(C(N)=O)cc(-c3ccc(CN4CCOCC4)cc3)nc12] | A | — |
| | Isomer 1 | | |
| | 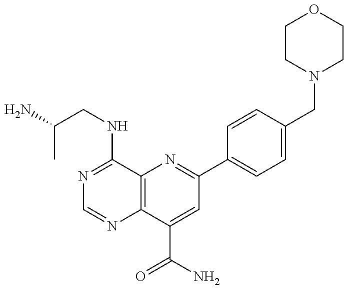 [SMILES: C[C@H](N)CNc1ncnc2c(C(N)=O)cc(-c3ccc(CN4CCOCC4)cc3)nc12] | | |
| | Isomer 2 | | |
| Example 26 | 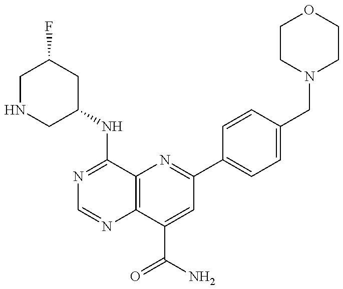 [SMILES: NC(=O)c1cc(-c2ccc(CN3CCOCC3)cc2)nc2c(N[C@@H]3CNC[C@H](F)C3)ncnc12] | A | — |
| | Isomer 1 | | |
| | 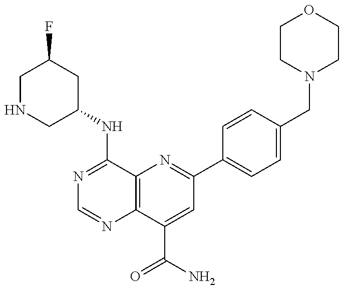 [SMILES: NC(=O)c1cc(-c2ccc(CN3CCOCC3)cc2)nc2c(N[C@@H]3CNC[C@@H](F)C3)ncnc12] | | |
| | Isomer 2 | | |
| | 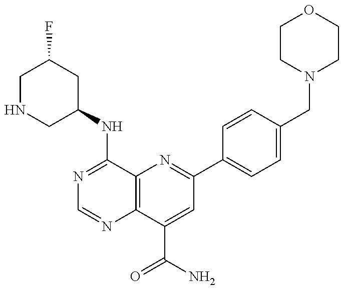 [SMILES: NC(=O)c1cc(-c2ccc(CN3CCOCC3)cc2)nc2c(N[C@H]3CNC[C@H](F)C3)ncnc12] | | |
| | Isomer 3 | | |
| | 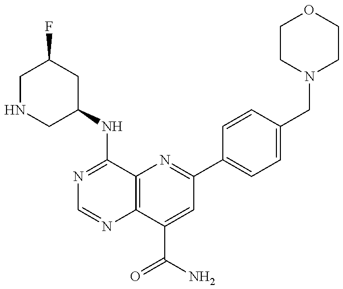 [SMILES: NC(=O)c1cc(-c2ccc(CN3CCOCC3)cc2)nc2c(N[C@H]3CNC[C@@H](F)C3)ncnc12] | | |
| | Isomer 4 | | |
| Example 27 | 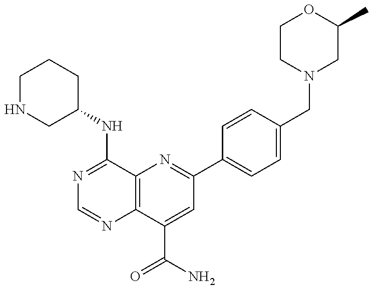 [SMILES: C[C@H]1CN(Cc2ccc(-c3cc(C(N)=O)c4ncnc(N[C@H]5CCCNC5)c4n3)cc2)CCO1] | A | A |
| Example 28 | 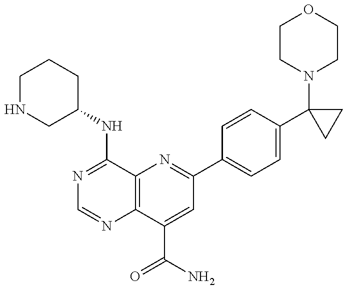 [SMILES: NC(=O)c1cc(-c2ccc(C3(N4CCOCC4)CC3)cc2)nc2c(N[C@H]3CCCNC3)ncnc12] | A | A |
| Example 29 | 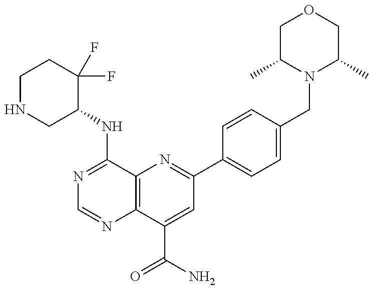 [SMILES: C[C@@H]1COC[C@H](C)N1Cc1ccc(-c2cc(C(N)=O)c3ncnc(N[C@@H]4CNCCC4(F)F)c3n2)cc1] | A | C |
| Example 30 | 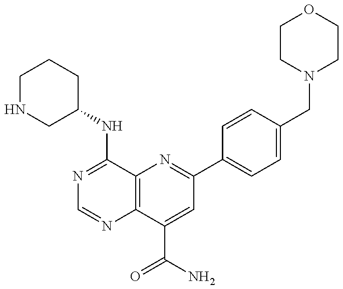 [SMILES: NC(=O)c1cc(-c2ccc(CN3CCOCC3)cc2)nc2c(N[C@H]3CCCNC3)ncnc12] | A | A |
| Example 31 | 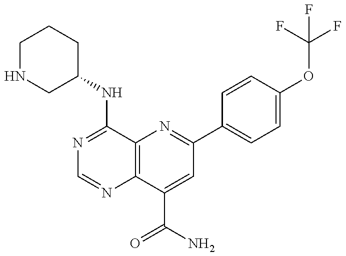 [SMILES: NC(=O)c1cc(-c2ccc(OC(F)(F)F)cc2)nc2c(N[C@H]3CCCNC3)ncnc12] | A | B |
| Example 32 | 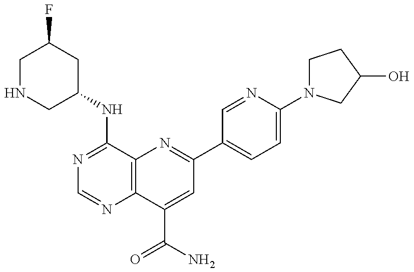 [SMILES: NC(=O)c1cc(-c2ccc(N3CCC(O)C3)nc2)nc2c(N[C@@H]3CNC[C@@H](F)C3)ncnc12] | A | C |
| Example 33 | 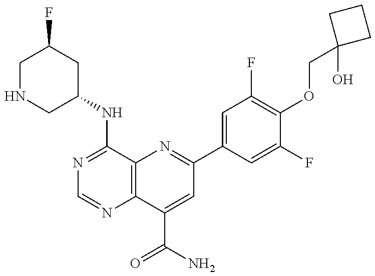 [SMILES: NC(=O)c1cc(-c2cc(F)c(OCC3(O)CCC3)c(F)c2)nc2c(N[C@@H]3CNC[C@@H](F)C3)ncnc12] | A | A |
| Example 34 | 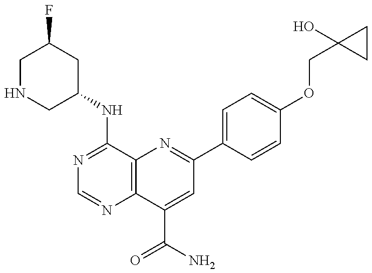 [SMILES: NC(=O)c1cc(-c2ccc(OCC3(O)CC3)cc2)nc2c(N[C@@H]3CNC[C@@H](F)C3)ncnc12] | A | A |
| Example 35 | 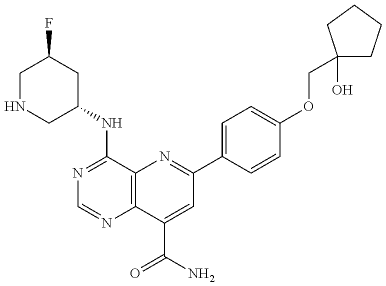 [SMILES: NC(=O)c1cc(-c2ccc(OCC3(O)CCCC3)cc2)nc2c(N[C@@H]3CNC[C@@H](F)C3)ncnc12] | A | B |
| Example 36 | 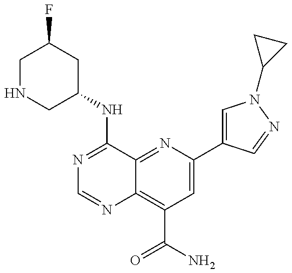 [SMILES: NC(=O)c1cc(-c2cnn(C3CC3)c2)nc2c(N[C@@H]3CNC[C@@H](F)C3)ncnc12] | A | B |
| Example 37 | 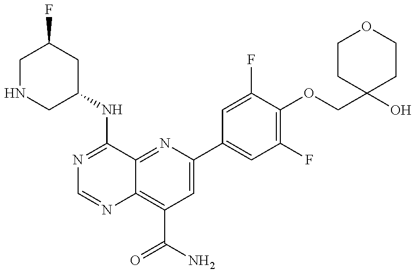 [SMILES: NC(=O)c1cc(-c2cc(F)c(OCC3(O)CCOCC3)c(F)c2)nc2c(N[C@@H]3CNC[C@@H](F)C3)ncnc12] | A | A |
| Example 38 | 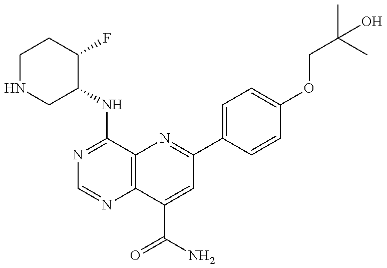 [SMILES: CC(C)(O)COc1ccc(-c2cc(C(N)=O)c3ncnc(N[C@@H]4CNCC[C@@H]4F)c3n2)cc1] | A | A |
| Example 39 | 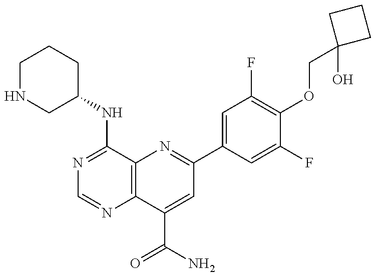 [SMILES: NC(=O)c1cc(-c2cc(F)c(OCC3(O)CCC3)c(F)c2)nc2c(N[C@H]3CCCNC3)ncnc12] | A | A |
| Example 40 | 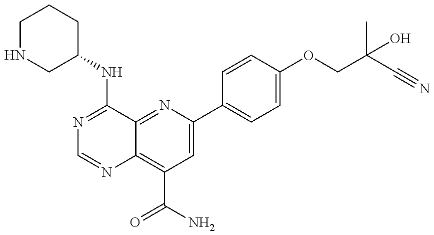 [SMILES: CC(O)(C#N)COc1ccc(-c2cc(C(N)=O)c3ncnc(N[C@H]4CCCNC4)c3n2)cc1] | A | C |
| Example 41 | 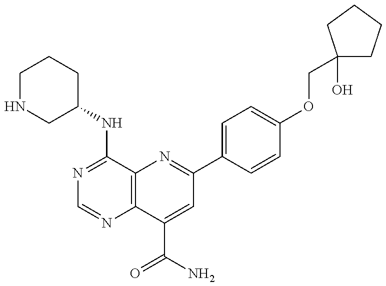 [SMILES: NC(=O)c1cc(-c2ccc(OCC3(O)CCCC3)cc2)nc2c(N[C@H]3CCCNC3)ncnc12] | A | A |
| Example 42 | 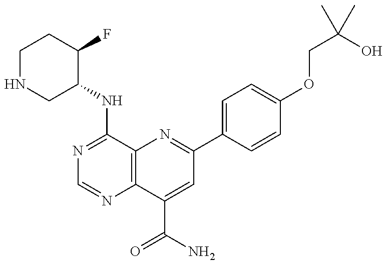 [SMILES: CC(C)(O)COc1ccc(-c2cc(C(N)=O)c3ncnc(N[C@@H]4CNCC[C@H]4F)c3n2)cc1] | A | A |
| Example 43 | 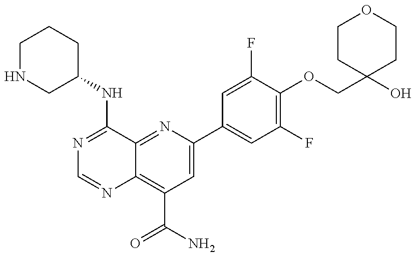 [SMILES: NC(=O)c1cc(-c2cc(F)c(OCC3(O)CCOCC3)c(F)c2)nc2c(N[C@H]3CCCNC3)ncnc12] | A | A |
| Example 44 | 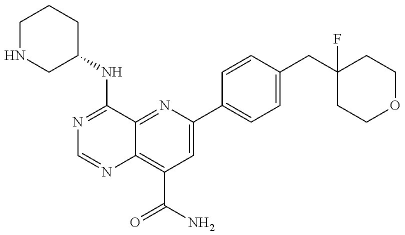 [SMILES: NC(=O)c1cc(-c2ccc(CC3(F)CCOCC3)cc2)nc2c(N[C@H]3CCCNC3)ncnc12] | A | A |
| Example 45 | 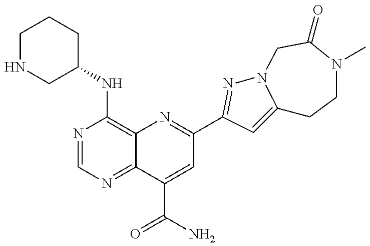 [SMILES: CN1CCc2cc(-c3cc(C(N)=O)c4ncnc(N[C@H]5CCCNC5)c4n3)nn2CC1=O] | A | C |
| Example 46 | 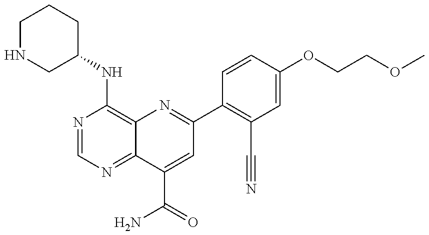 [SMILES: COCCOc1ccc(-c2cc(C(N)=O)c3ncnc(N[C@H]4CCCNC4)c3n2)c(C#N)c1] | A | C |
| Example 47 | 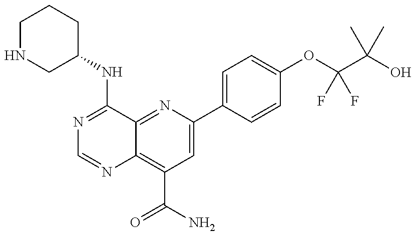 [SMILES: CC(C)(O)C(F)(F)Oc1ccc(-c2cc(C(N)=O)c3ncnc(N[C@H]4CCCNC4)c3n2)cc1] | A | B |
| Example 48 | 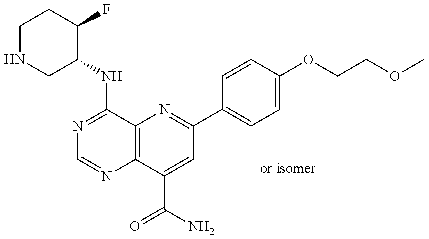 [SMILES: COCCOc1ccc(-c2cc(C(N)=O)c3ncnc(N[C@@H]4CNCC[C@H]4F)c3n2)cc1] | A | A |
| Example 49 | 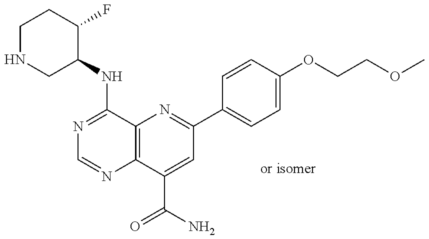 [SMILES: COCCOc1ccc(-c2cc(C(N)=O)c3ncnc(N[C@H]4CNCC[C@@H]4F)c3n2)cc1] | C | C |
| Example 50 | 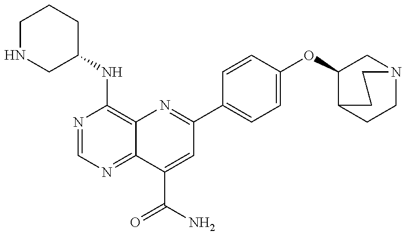 [SMILES: NC(=O)c1cc(-c2ccc(O[C@H]3CN4CCC3CC4)cc2)nc2c(N[C@H]3CCCNC3)ncnc12] | A | C |
| Example 51 | 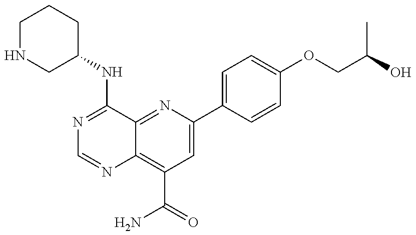 [SMILES: C[C@@H](O)COc1ccc(-c2cc(C(N)=O)c3ncnc(N[C@H]4CCCNC4)c3n2)cc1] | A | A |
| Example 52 | 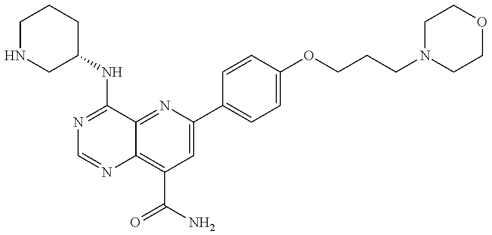 [SMILES: NC(=O)c1cc(-c2ccc(OCCCN3CCOCC3)cc2)nc2c(N[C@H]3CCCNC3)ncnc12] | A | A |
| Example 53 | 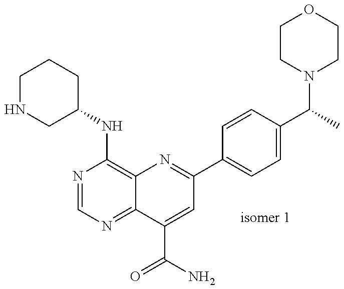 [SMILES: C[C@H](c1ccc(-c2cc(C(N)=O)c3ncnc(N[C@H]4CCCNC4)c3n2)cc1)N1CCOCC1] | A | B |
| | 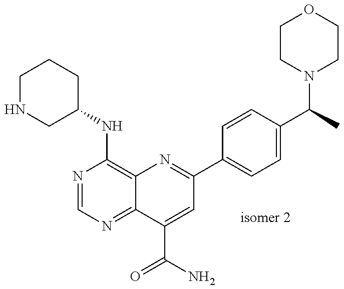 [SMILES: C[C@@H](c1ccc(-c2cc(C(N)=O)c3ncnc(N[C@H]4CCCNC4)c3n2)cc1)N1CCOCC1] | | |
| Example 54 | 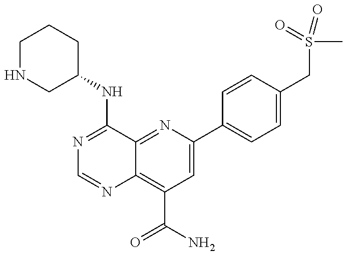 [SMILES: CS(=O)(=O)Cc1ccc(-c2cc(C(N)=O)c3ncnc(N[C@H]4CCCNC4)c3n2)cc1] | A | C |
| Example 55 | 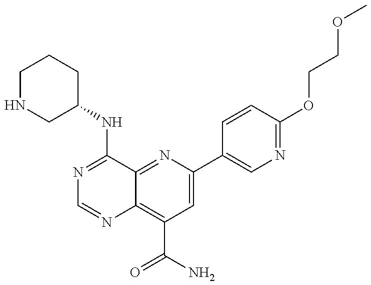 [SMILES: COCCOc1ccc(-c2cc(C(N)=O)c3ncnc(N[C@H]4CCCNC4)c3n2)cn1] | A | C |
| Example 56 | 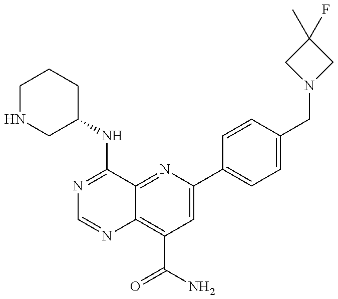 [SMILES: CC1(F)CN(Cc2ccc(-c3cc(C(N)=O)c4ncnc(N[C@H]5CCCNC5)c4n3)cc2)C1] | A | A |
| Example 57 | 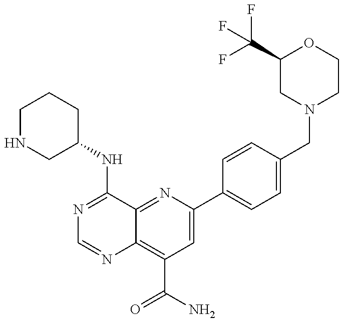 [SMILES: NC(=O)c1cc(-c2ccc(CN3CCO[C@H](C(F)(F)F)C3)cc2)nc2c(N[C@H]3CCCNC3)ncnc12] | A | A |
| Example 58 | 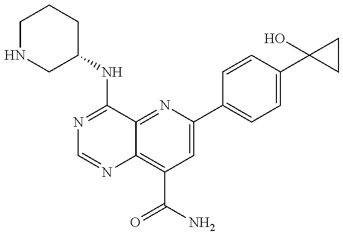 [SMILES: NC(=O)c1cc(-c2ccc(C3(O)CC3)cc2)nc2c(N[C@H]3CCCNC3)ncnc12] | A | A |
| Example 59 | 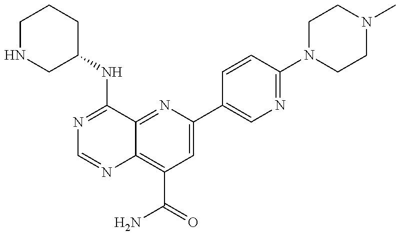 [SMILES: CN1CCN(c2ccc(-c3cc(C(N)=O)c4ncnc(N[C@H]5CCCNC5)c4n3)cn2)CC1] | A | A |
| Example 60 | 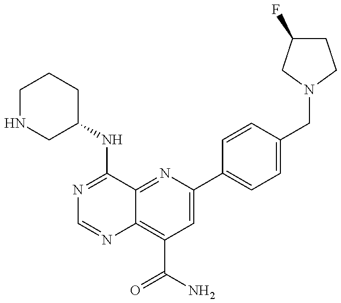 [SMILES: NC(=O)c1cc(-c2ccc(CN3CC[C@H](F)C3)cc2)nc2c(N[C@H]3CCCNC3)ncnc12] | A | NA |
| Example 61 | 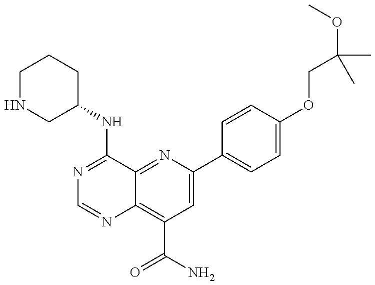 [SMILES: COC(C)(C)COc1ccc(-c2cc(C(N)=O)c3ncnc(N[C@H]4CCCNC4)c3n2)cc1] | A | A |
| Example 62 | 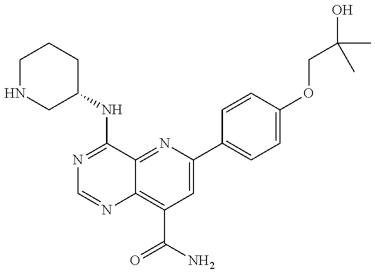 [SMILES: CC(C)(O)COc1ccc(-c2cc(C(N)=O)c3ncnc(N[C@H]4CCCNC4)c3n2)cc1] | A | A |
| Example 63 | 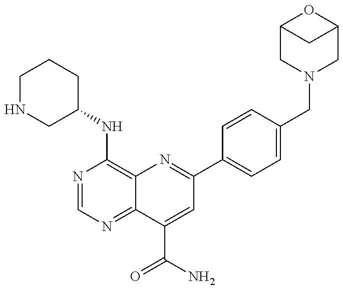 [SMILES: NC(=O)c1cc(-c2ccc(CN3CC4CC(C3)O4)cc2)nc2c(N[C@H]3CCCNC3)ncnc12] | A | A |
| Example 64 | 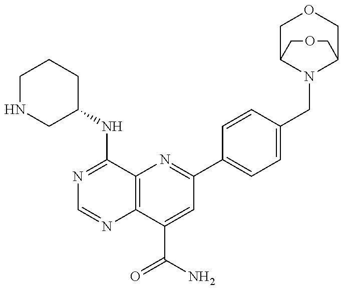 [SMILES: NC(=O)c1cc(-c2ccc(CN3C4COCC3COC4)cc2)nc2c(N[C@H]3CCCNC3)ncnc12] | A | A |
| Example 65 | 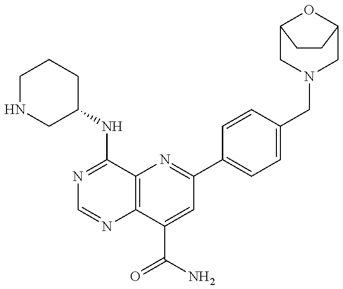 [SMILES: NC(=O)c1cc(-c2ccc(CN3CC4CCC(C3)O4)cc2)nc2c(N[C@H]3CCCNC3)ncnc12] | A | A |
| Example 66 | 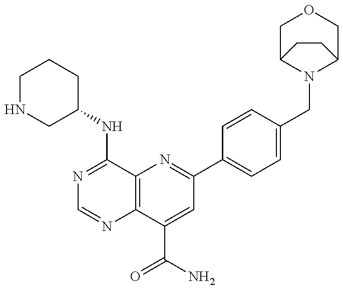 [SMILES: NC(=O)c1cc(-c2ccc(CN3C4CCC3COC4)cc2)nc2c(N[C@H]3CCCNC3)ncnc12] | A | A |
| Example 67 | 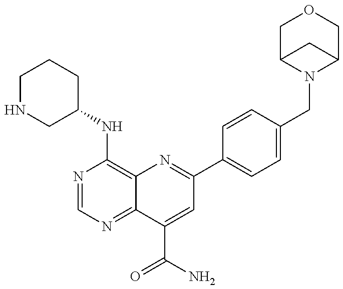 [SMILES: NC(=O)c1cc(-c2ccc(CN3C4COCC3C4)cc2)nc2c(N[C@H]3CCCNC3)ncnc12] | A | C |
| Example 68 | 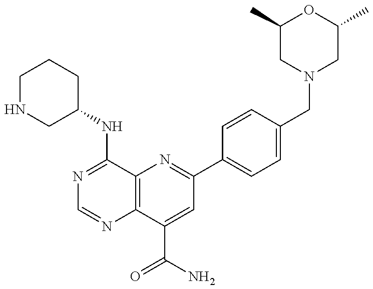 [SMILES: C[C@@H]1CN(Cc2ccc(-c3cc(C(N)=O)c4ncnc(N[C@H]5CCCNC5)c4n3)cc2)C[C@@H](C)O1] | A | B |
| Example 69 | 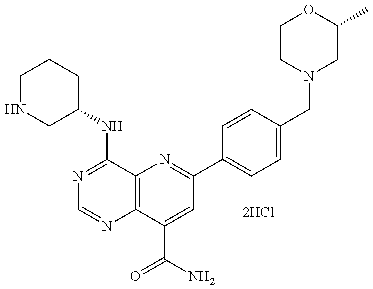 [SMILES: C[C@@H]1CN(Cc2ccc(-c3cc(C(N)=O)c4ncnc(N[C@H]5CCCNC5)c4n3)cc2)CCO1] | A | C |
| Example 70 | 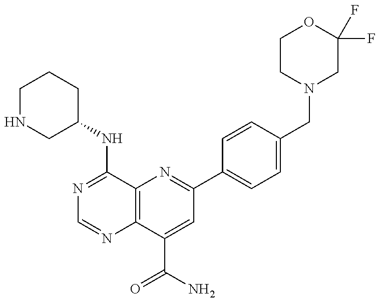 [SMILES: NC(=O)c1cc(-c2ccc(CN3CCOC(F)(F)C3)cc2)nc2c(N[C@H]3CCCNC3)ncnc12] | A | B |
| Example 71 | 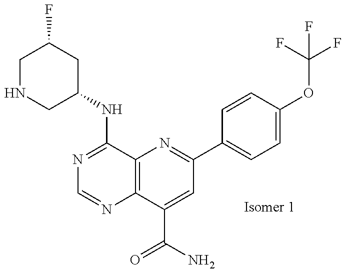 [SMILES: NC(=O)c1cc(-c2ccc(OC(F)(F)F)cc2)nc2c(N[C@@H]3CNC[C@H](F)C3)ncnc12] | A | NA |
| | cis assumed; | | |
| | abs assumed | | |
| | 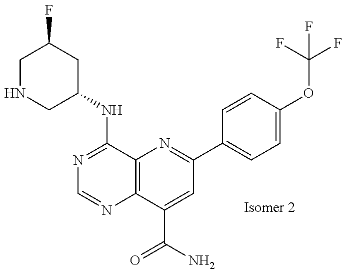 [SMILES: NC(=O)c1cc(-c2ccc(OC(F)(F)F)cc2)nc2c(N[C@@H]3CNC[C@@H](F)C3)ncnc12] | NA | NA |
| | trans assumed; | | |
| | abs assumed | | |
| | 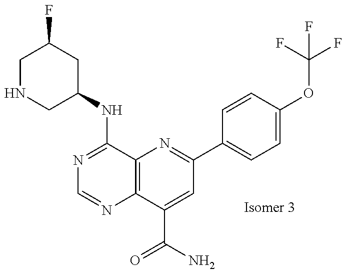 [SMILES: NC(=O)c1cc(-c2ccc(OC(F)(F)F)cc2)nc2c(N[C@H]3CNC[C@@H](F)C3)ncnc12] | NA | NA |
| | cis assumed; | | |
| | abs assumed | | |
| | 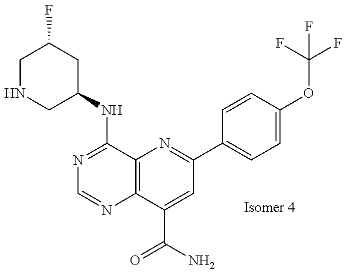 [SMILES: NC(=O)c1cc(-c2ccc(OC(F)(F)F)cc2)nc2c(N[C@H]3CNC[C@H](F)C3)ncnc12] | NA | NA |
| | trans assumed; | | |
| | abs assumed | | |
| Example 72 | 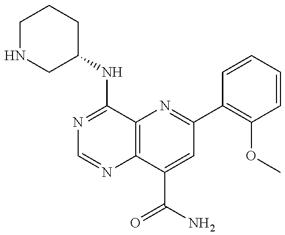 [SMILES: COc1ccccc1-c1cc(C(N)=O)c2ncnc(N[C@H]3CCCNC3)c2n1] | A | C |
| Example 73 | 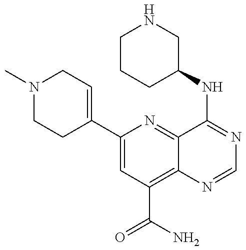 [SMILES: CN1CC=C(c2cc(C(N)=O)c3ncnc(N[C@H]4CCCNC4)c3n2)CC1] | C | NA |
| Example 74 | 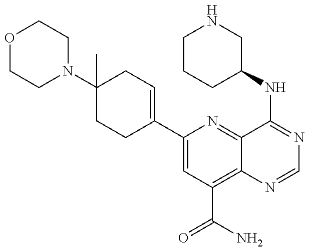 [SMILES: CC1(N2CCOCC2)CC=C(c2cc(C(N)=O)c3ncnc(N[C@H]4CCCNC4)c3n2)CC1] | A | C |
| Example 75 | 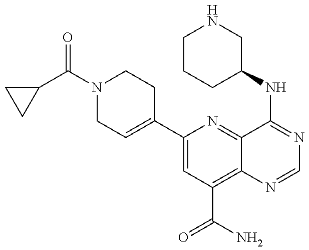 [SMILES: NC(=O)c1cc(C2=CCN(C(=O)C3CC3)CC2)nc2c(N[C@H]3CCCNC3)ncnc12] | A | B |
| Example 76 | 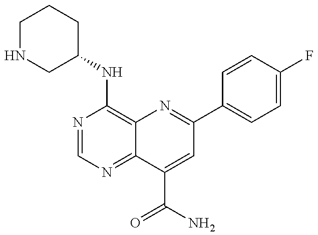 [SMILES: NC(=O)c1cc(-c2ccc(F)cc2)nc2c(N[C@H]3CCCNC3)ncnc12] | A | A |
| Example 77 | 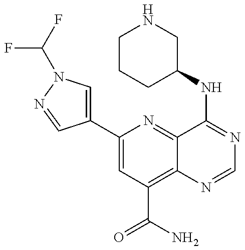 [SMILES: NC(=O)c1cc(-c2cnn(C(F)F)c2)nc2c(N[C@H]3CCCNC3)ncnc12] | A | NA |
| Example 78 | 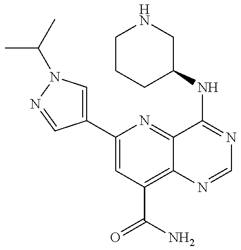 [SMILES: CC(C)n1cc(-c2cc(C(N)=O)c3ncnc(N[C@H]4CCCNC4)c3n2)cn1] | A | NA |
| Example 79 | 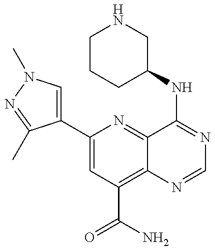 [SMILES: Cc1nn(C)cc1-c1cc(C(N)=O)c2ncnc(N[C@H]3CCCNC3)c2n1] | A | C |
| Example 80 | 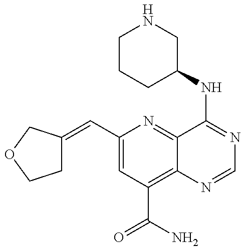 [SMILES: NC(=O)c1cc(/C=C2\CCOC2)nc2c(N[C@H]3CCCNC3)ncnc12] | B | NA |
| Example 81 | 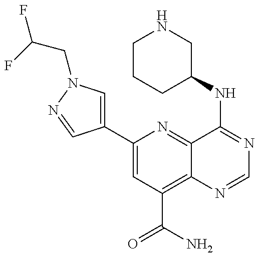 [SMILES: NC(=O)c1cc(-c2cnn(CC(F)F)c2)nc2c(N[C@H]3CCCNC3)ncnc12] | A | C |
| Example 82 | 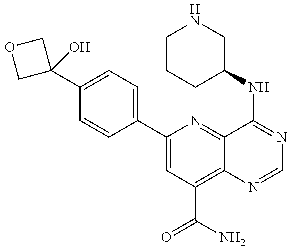 [SMILES: NC(=O)c1cc(-c2ccc(C3(O)COC3)cc2)nc2c(N[C@H]3CCCNC3)ncnc12] | A | B |
| Example 83 | 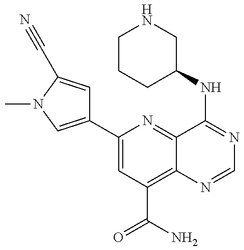 [SMILES: Cn1cc(-c2cc(C(N)=O)c3ncnc(N[C@H]4CCCNC4)c3n2)cc1C#N] | A | A |
| Example 84 | 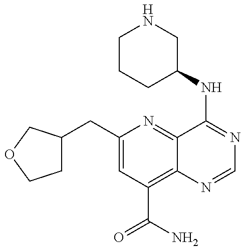 [SMILES: NC(=O)c1cc(CC2CCOC2)nc2c(N[C@H]3CCCNC3)ncnc12] | C | NA |
| Example 85 | 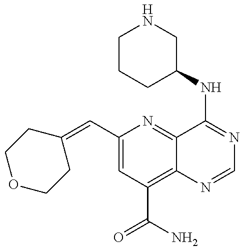 [SMILES: NC(=O)c1cc(C=C2CCOCC2)nc2c(N[C@H]3CCCNC3)ncnc12] | C | NA |
| Example 86 | 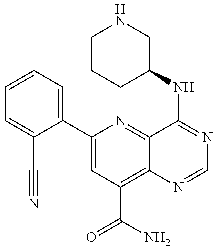 [SMILES: N#Cc1ccccc1-c1cc(C(N)=O)c2ncnc(N[C@H]3CCCNC3)c2n1] | A | B |
| Example 87 | 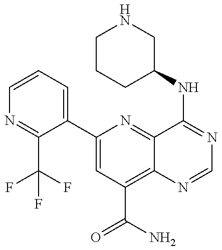 [SMILES: NC(=O)c1cc(-c2cccnc2C(F)(F)F)nc2c(N[C@H]3CCCNC3)ncnc12] | B | NA |
| Example 88 | 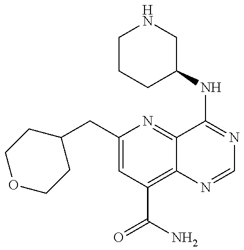 [SMILES: NC(=O)c1cc(CC2CCOCC2)nc2c(N[C@H]3CCCNC3)ncnc12] | C | NA |

1. A compound of formula I 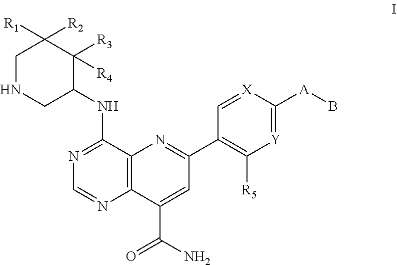
wherein:
R₁, R₂, R₃ and R₄ are each independently selected from the group consisting of H and halogen;
R₅ is selected from the group consisting of H, halogen, —O—C₁-C₆ alkyl, C₁-C₆ alkyl, C₁-C₆ haloalkyl and —CN;
X is selected from the group consisting of N and CR₆
Y is selected from the group consisting of N and CR₇
R₆ and R₇ are each independently selected from the group consisting of H, halogen and C₁-C₆ alkyl;
A is absent or selected from the group consisting of (—CH₂—)ₙ, —O—(—CH₂—)ₒ-, —O— and 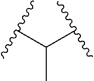 [SMILES: *C(*)C]
n is selected from the group consisting of 1, 2, 3 and 4;
is selected from the group consisting of 1, 2, 3 and 4;
B is absent or selected from the group consisting of H, CN, halogen, optionally substituted C₆-C₁₄ aryl, optionally substituted C₂-C₁₄ heteroaryl, optionally substituted C₁-C₁₄ heterocyclic, optionally substituted C₁-C₈ alkyl, C₁-C₈ haloalkyl, optionally substituted C₁-C₈ alkyl interrupted by 1-4 heteroatoms, and optionally substituted C₃-C₁₄ cycloalkyl;
or a pharmaceutically acceptable salt, prodrugs, enantiomer, mixture of enantiomers, diastereomers or mixture of diastereomers.

2. The compound according to claim 1 having the formula Ia

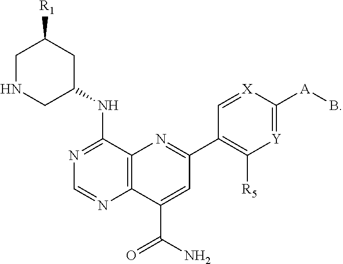

3. The compound according to claim 2 having the formula Ib

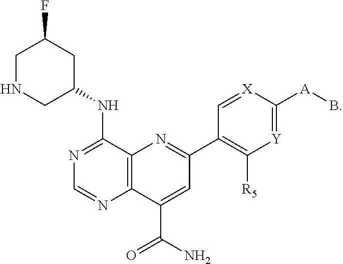

4. The compound according to claim 1 having the formula Ic

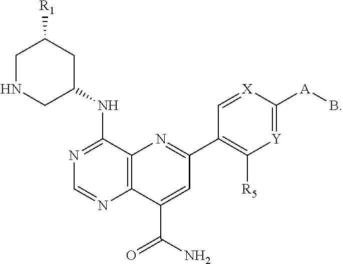

5. The compound according to claim 1 having the formula Id

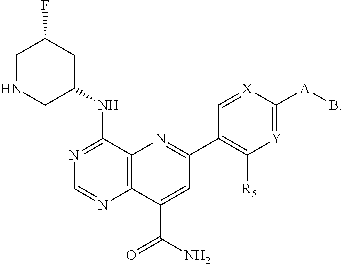

6. The compound according to claim 1 having the formula Ie

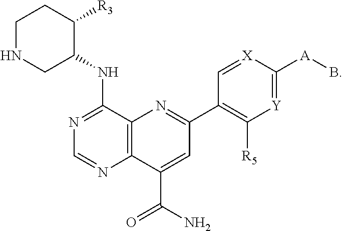

7. The compound according to claim 4 having the formula If

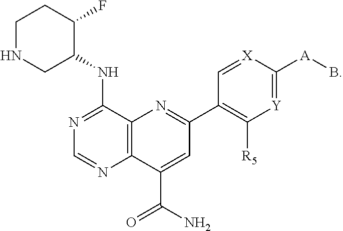

8. The compound according to claim 1 having the formula Ig

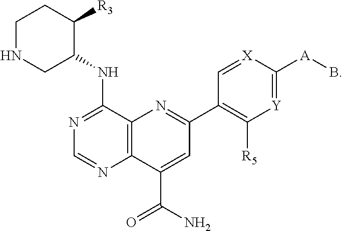

9. The compound according to claim 6 having the formula Ih

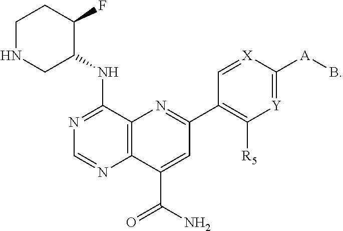

10. The compound according to claim 1 having the formula Il

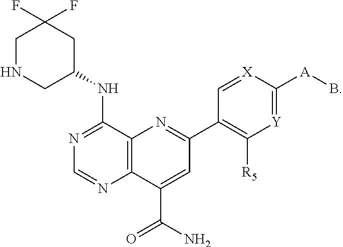

11. The compound according to claim 1 having the formula Ij

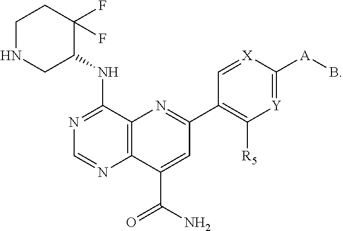

12. The compound according to claim 1 having the formula Ik

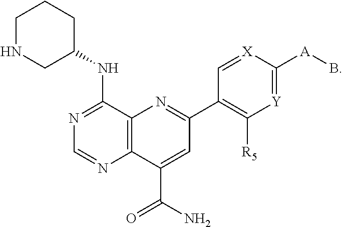

13. The compound according to claim 1 wherein R₁, R₂, R₃ and R₄ are each H.

14. The compound according to claim 1 wherein R₁, R₃ and R₄ are H and R₂ is a halogen.

15. The compound according to claim 14 wherein R₂ is F.

16. The compound according to claim 1 wherein R₁, R₂, and R₃ are H and R₄ is halogen.

17. The compound according to claim 16 where R₄ is F.

18. The compound according to claim 1 wherein R₁ and R₂ are halogen and R₃ and R₄ are H.

19. The compound according to claim 18 wherein R₁ and R₂ are F.

20. The compound according to claim 1 wherein R₁ and R₂ are H and R₃ and R₄ are halogen.

21. The compound according to claim 20 wherein R₃ and R₄ are F.

22. The compound according to claim 1 wherein R₅ is selected from the group consisting of H, —CN and —CF₃.

23. The compound according to claim 1 wherein R₅ is —CN.

24. The compound according to claim 1 wherein -A-B is selected from the group consisting of 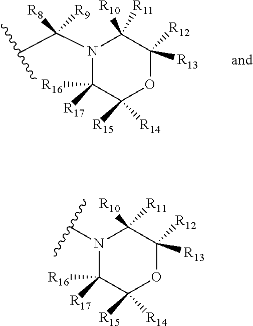
wherein R₈ and R₉ are each independently selected from the group consisting of H and C₁-C₆ alkyl; R₈ and R₉ together with the carbon to which they are attached can form a ring having 3-6 carbon atoms; R₁₀, R₁₁, R₁₂, R₁₃, R₁₄, R₁₅, R₁₆ and R₁₇ are each independently selected from the group consisting of H, C₁-C₆ alkyl, C₁-C₆ haloalkyl, halogen and —CN.

25. The compound according to claim 24 wherein R₈ and R₉ are H.

26. The compound according to claim 24 wherein R₈ and R₉ together with the carbon they are attached form a cyclopropyl ring.

27. The compound according to claim 24 wherein R₁₀, R₁₁, R₁₆ and R₁₇ are each independently selected from the group consisting of H and —CH₃; and R₁₂, R₁₃, R₁₄ and R₁₅ are H.

28. The compound according to claim 24 wherein R₁₂ and R₁₃ are both F and R₁₀, R₁₁, R₁₄, R₁₅, R₁₆ and R₁₇ are H.

29. The compound according to claim 24 wherein R₁₀ and R₁₁ are each independently selected from the group consisting of H and —CN; and R₁₂, R₁₃, R₁₄, R₁₅, R₁₆ and R₁₇ are H.

30. The compound according to claim 24 wherein R₁₀ and R₁₁ are each independently selected from the group consisting of H and —CF₃; and R₁₂, R₁₃, R₁₄, R₁₅, R₁₆ and R₁₇ are H.

31. The compound according to claim 24 wherein R₁₂ and R₁₃ are each independently selected from the group consisting of H and —CN; and R₁₀, R₁₁, R₁₄, R₁₅, R₁₆ and R₁₇ are H.

32. The compound according to claim 24 wherein R₁₂ and R₁₃ are each independently selected from the group consisting of H and —CF₃; and R₁₀, R₁₁, R₁₄, R₁₅, R₁₆ and R₁₇ are H.

33. The compound according to claim 24 wherein R₈, R₉, R₁₀, R₁₁, R₁₂, R₁₃, R₁₄, R₁₅, R₁₆ and R₁₇ are H.

34. The compound according to claim 24 wherein R₁₀, R₁₁, R₁₂, R₁₃, R₁₄, R₁₅, R₁₆ and R₁₇ are H.

35. The compound according to claim 24 wherein R₁₂ and R₁₃ are each independently selected from the group consisting of H and —CH₃; and R₁₀, R₁₁, R₁₄, R₁₅, R₁₆ and R₁₇ are H.

36. The compound according to claim 1 wherein -A-B is selected from the group consisting of

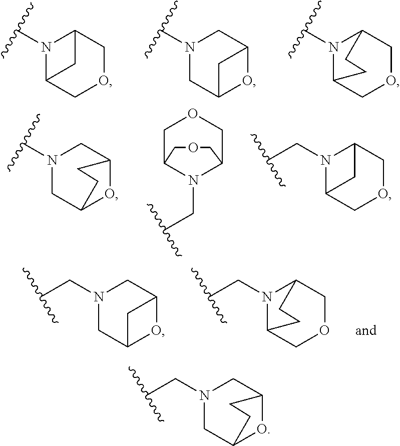

37. The compound according to claim 1 wherein -A-B is selected from the group consisting of 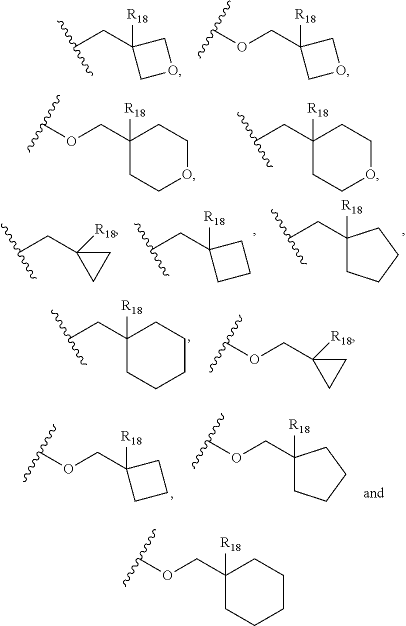
wherein R₁₈ is selected from the group consisting of H, —OH, halogen, C₁-C₆ alkyl, —O—(C₁-C₆ alkyl) and C₁-C₆ haloalkyl.

38. The compound according to claim 1 wherein -A-B is selected from the group consisting of 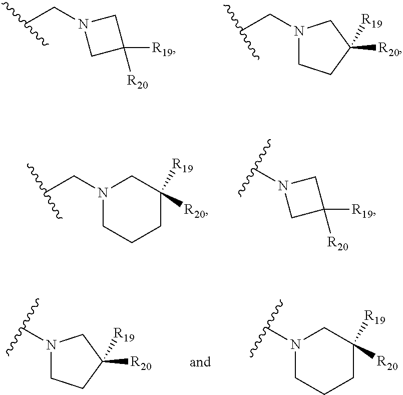
wherein R₁₉ and R₂₀ are each independently selected from the group consisting of H, —OH, halogen, C₁-C₆ alkyl, —O—(C₁-C₆ alkyl) and C₁-C₆ haloalkyl.

39. The compound according to claim 1 wherein -A-B is selected from the group consisting of 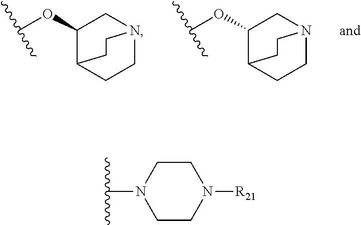
wherein R₂₁ is selected from the group consisting of H and C₁-C₆ alkyl.

40. The compound according to claim 1 wherein -A-B is selected from the group consisting of —OCH₃, —CN, —CH₂SO₂CH₃, —OCF₃, —CF₃, —CHF₂,

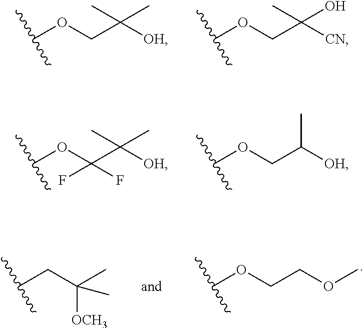

41-62. (canceled)

63. The compound according to claim 1 selected from the group consisting of

| # | Structure |
|---|---|
| Example 1 | 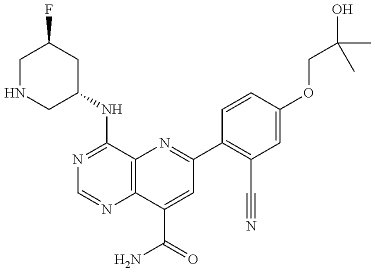 [SMILES: CC(C)(O)COc1ccc(-c2cc(C(N)=O)c3ncnc(N[C@@H]4CNC[C@@H](F)C4)c3n2)c(C#N)c1] |
| Example 10 | 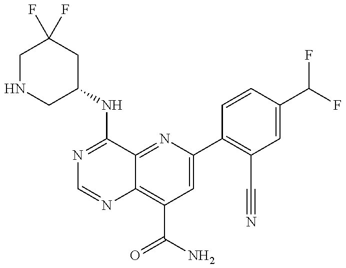 [SMILES: N#Cc1cc(C(F)F)ccc1-c1cc(C(N)=O)c2ncnc(N[C@@H]3CNCC(F)(F)C3)c2n1] |
| Example 11 | 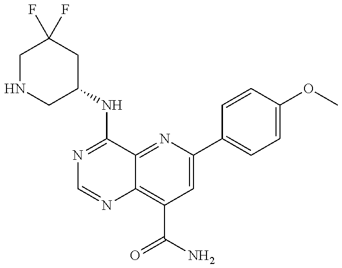 [SMILES: COc1ccc(-c2cc(C(N)=O)c3ncnc(N[C@@H]4CNCC(F)(F)C4)c3n2)cc1] |
| Example 12 | 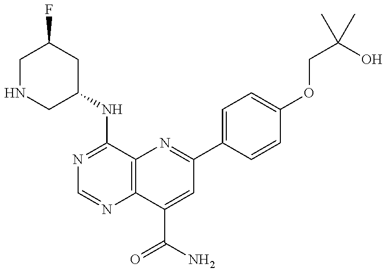 [SMILES: CC(C)(O)COc1ccc(-c2cc(C(N)=O)c3ncnc(N[C@@H]4CNC[C@@H](F)C4)c3n2)cc1] |
| Example 14 | 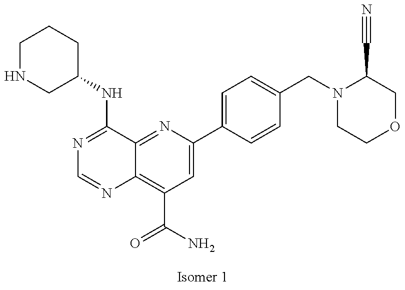 [SMILES: N#C[C@H]1COCCN1Cc1ccc(-c2cc(C(N)=O)c3ncnc(N[C@H]4CCCNC4)c3n2)cc1] |
| | 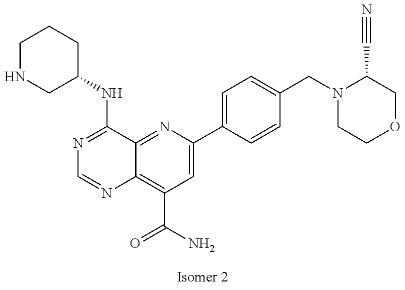 [SMILES: N#C[C@@H]1COCCN1Cc1ccc(-c2cc(C(N)=O)c3ncnc(N[C@H]4CCCNC4)c3n2)cc1] |
| Example 15 | 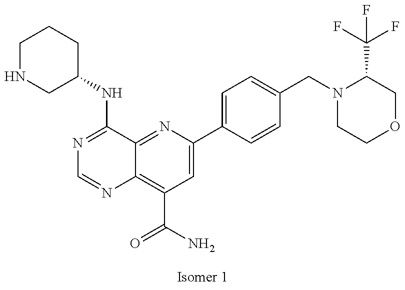 [SMILES: NC(=O)c1cc(-c2ccc(CN3CCOC[C@H]3C(F)(F)F)cc2)nc2c(N[C@H]3CCCNC3)ncnc12] |
| | 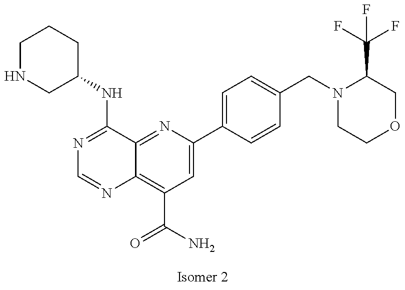 [SMILES: NC(=O)c1cc(-c2ccc(CN3CCOC[C@@H]3C(F)(F)F)cc2)nc2c(N[C@H]3CCCNC3)ncnc12] |
| Example 16 | 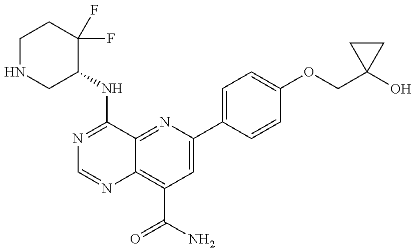 [SMILES: NC(=O)c1cc(-c2ccc(OCC3(O)CC3)cc2)nc2c(N[C@@H]3CNCCC3(F)F)ncnc12] |
| Example 22 | 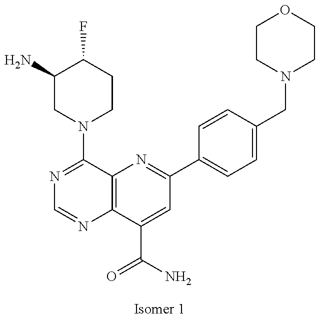 [SMILES: NC(=O)c1cc(-c2ccc(CN3CCOCC3)cc2)nc2c(N3CC[C@@H](F)[C@H](N)C3)ncnc12] |
| | 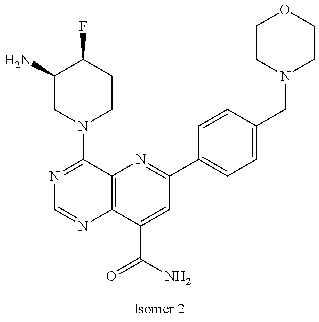 [SMILES: NC(=O)c1cc(-c2ccc(CN3CCOCC3)cc2)nc2c(N3CC[C@H](F)[C@H](N)C3)ncnc12] |
| | 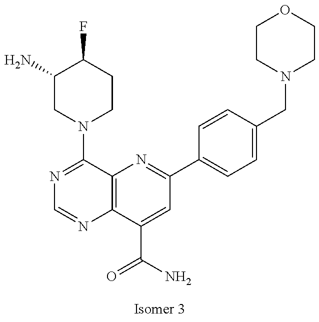 [SMILES: NC(=O)c1cc(-c2ccc(CN3CCOCC3)cc2)nc2c(N3CC[C@H](F)[C@@H](N)C3)ncnc12] |
| | 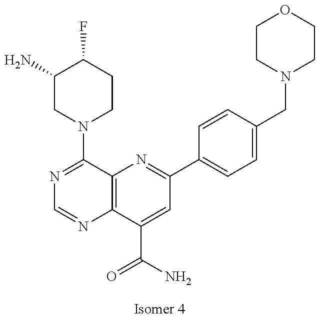 [SMILES: NC(=O)c1cc(-c2ccc(CN3CCOCC3)cc2)nc2c(N3CC[C@@H](F)[C@@H](N)C3)ncnc12] |
| Example 24 | 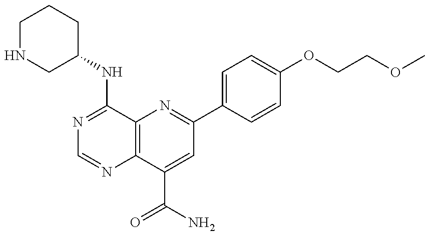 [SMILES: COCCOc1ccc(-c2cc(C(N)=O)c3ncnc(N[C@H]4CCCNC4)c3n2)cc1] |
| Example 26 | 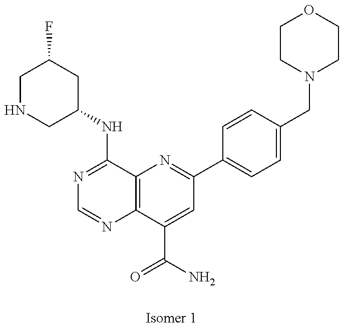 [SMILES: NC(=O)c1cc(-c2ccc(CN3CCOCC3)cc2)nc2c(N[C@@H]3CNC[C@H](F)C3)ncnc12] |
| | 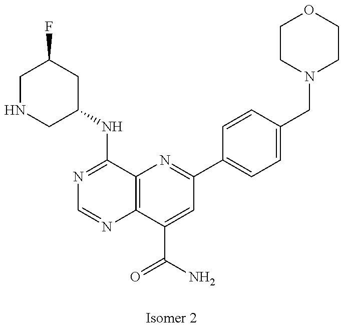 [SMILES: NC(=O)c1cc(-c2ccc(CN3CCOCC3)cc2)nc2c(N[C@@H]3CNC[C@@H](F)C3)ncnc12] |
| | 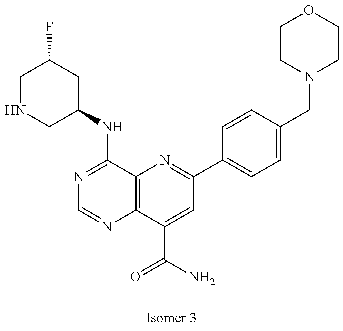 [SMILES: NC(=O)c1cc(-c2ccc(CN3CCOCC3)cc2)nc2c(N[C@H]3CNC[C@H](F)C3)ncnc12] |
| | 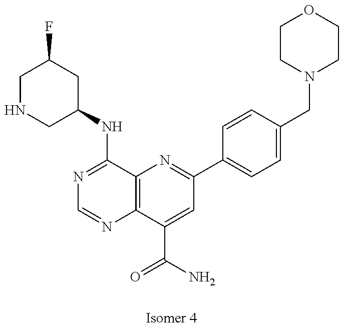 [SMILES: NC(=O)c1cc(-c2ccc(CN3CCOCC3)cc2)nc2c(N[C@H]3CNC[C@@H](F)C3)ncnc12] |
| Example 27 | 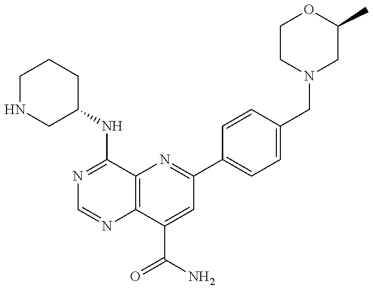 [SMILES: C[C@H]1CN(Cc2ccc(-c3cc(C(N)=O)c4ncnc(N[C@H]5CCCNC5)c4n3)cc2)CCO1] |
| Example 28 | 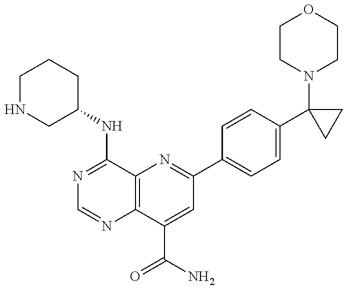 [SMILES: NC(=O)c1cc(-c2ccc(C3(N4CCOCC4)CC3)cc2)nc2c(N[C@H]3CCCNC3)ncnc12] |
| Example 29 | 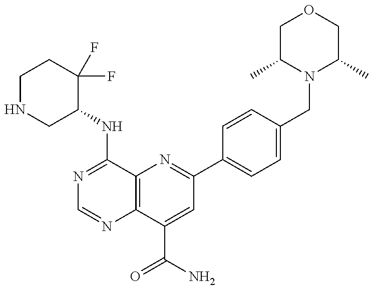 [SMILES: C[C@@H]1COC[C@H](C)N1Cc1ccc(-c2cc(C(N)=O)c3ncnc(N[C@@H]4CNCCC4(F)F)c3n2)cc1] |
| Example 30 | 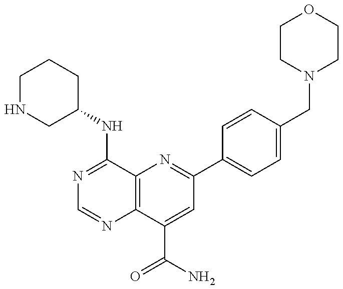 [SMILES: NC(=O)c1cc(-c2ccc(CN3CCOCC3)cc2)nc2c(N[C@H]3CCCNC3)ncnc12] |
| Example 31 | 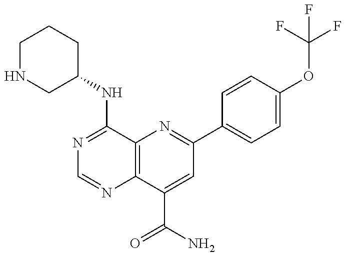 [SMILES: NC(=O)c1cc(-c2ccc(OC(F)(F)F)cc2)nc2c(N[C@H]3CCCNC3)ncnc12] |
| Example 32 | 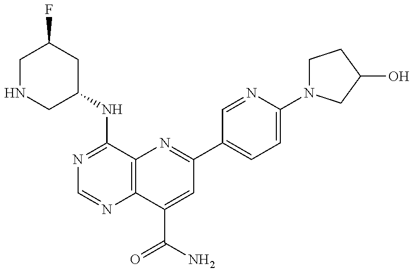 [SMILES: NC(=O)c1cc(-c2ccc(N3CCC(O)C3)nc2)nc2c(N[C@@H]3CNC[C@@H](F)C3)ncnc12] |
| Example 33 | 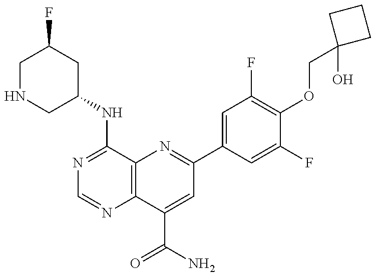 [SMILES: NC(=O)c1cc(-c2cc(F)c(OCC3(O)CCC3)c(F)c2)nc2c(N[C@@H]3CNC[C@@H](F)C3)ncnc12] |
| Example 34 | 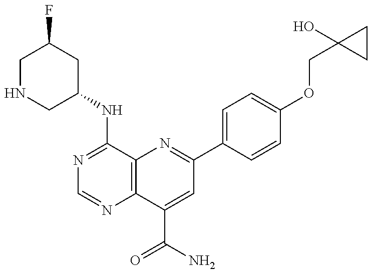 [SMILES: NC(=O)c1cc(-c2ccc(OCC3(O)CC3)cc2)nc2c(N[C@@H]3CNC[C@@H](F)C3)ncnc12] |
| Example 35 | 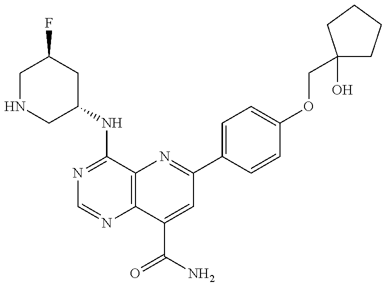 [SMILES: NC(=O)c1cc(-c2ccc(OCC3(O)CCCC3)cc2)nc2c(N[C@@H]3CNC[C@@H](F)C3)ncnc12] |
| Example 37 | 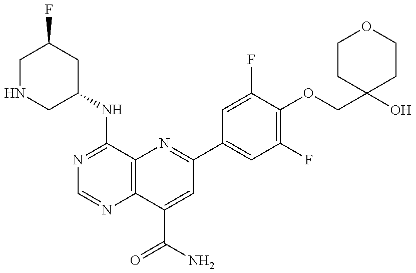 [SMILES: NC(=O)c1cc(-c2cc(F)c(OCC3(O)CCOCC3)c(F)c2)nc2c(N[C@@H]3CNC[C@@H](F)C3)ncnc12] |
| Example 38 | 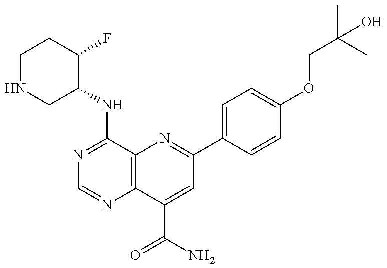 [SMILES: CC(C)(O)COc1ccc(-c2cc(C(N)=O)c3ncnc(N[C@@H]4CNCC[C@@H]4F)c3n2)cc1] |
| Example 39 | 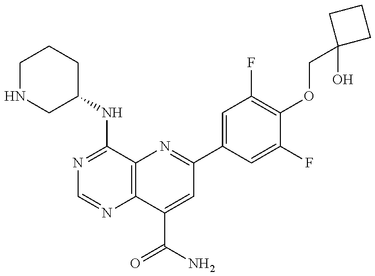 [SMILES: NC(=O)c1cc(-c2cc(F)c(OCC3(O)CCC3)c(F)c2)nc2c(N[C@H]3CCCNC3)ncnc12] |
| Example 40 | 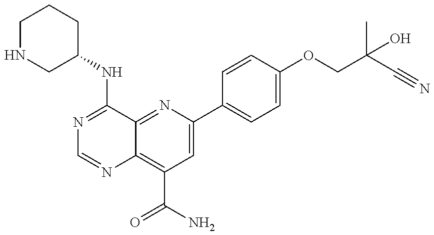 [SMILES: CC(O)(C#N)COc1ccc(-c2cc(C(N)=O)c3ncnc(N[C@H]4CCCNC4)c3n2)cc1] |
| Example 41 | 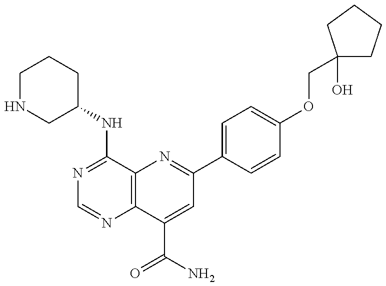 [SMILES: NC(=O)c1cc(-c2ccc(OCC3(O)CCCC3)cc2)nc2c(N[C@H]3CCCNC3)ncnc12] |
| Example 42 | 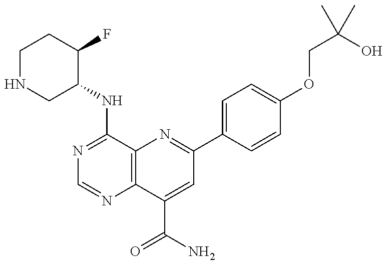 [SMILES: CC(C)(O)COc1ccc(-c2cc(C(N)=O)c3ncnc(N[C@@H]4CNCC[C@H]4F)c3n2)cc1] |
| Example 43 | 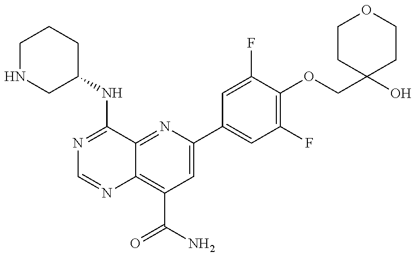 [SMILES: NC(=O)c1cc(-c2cc(F)c(OCC3(O)CCOCC3)c(F)c2)nc2c(N[C@H]3CCCNC3)ncnc12] |
| Example 44 | 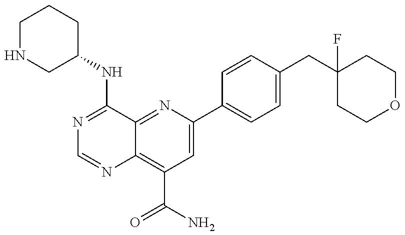 [SMILES: NC(=O)c1cc(-c2ccc(CC3(F)CCOCC3)cc2)nc2c(N[C@H]3CCCNC3)ncnc12] |
| Example 46 | 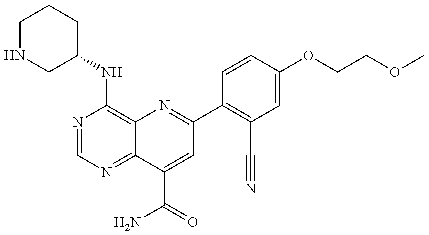 [SMILES: COCCOc1ccc(-c2cc(C(N)=O)c3ncnc(N[C@H]4CCCNC4)c3n2)c(C#N)c1] |
| Example 47 | 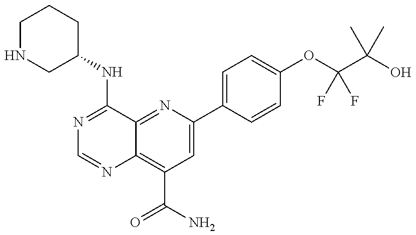 [SMILES: CC(C)(O)C(F)(F)Oc1ccc(-c2cc(C(N)=O)c3ncnc(N[C@H]4CCCNC4)c3n2)cc1] |
| Example 48 | 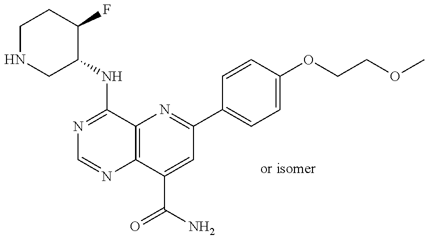 [SMILES: COCCOc1ccc(-c2cc(C(N)=O)c3ncnc(N[C@@H]4CNCC[C@H]4F)c3n2)cc1] |
| Example 49 | 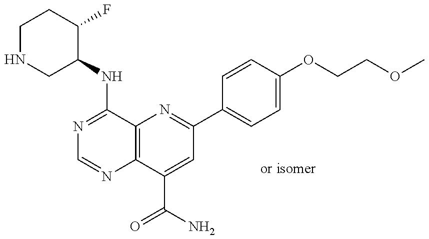 [SMILES: COCCOc1ccc(-c2cc(C(N)=O)c3ncnc(N[C@H]4CNCC[C@@H]4F)c3n2)cc1] |
| Example 50 | 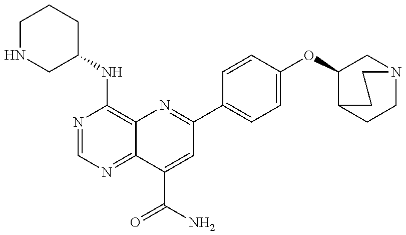 [SMILES: NC(=O)c1cc(-c2ccc(O[C@H]3CN4CCC3CC4)cc2)nc2c(N[C@H]3CCCNC3)ncnc12] |
| Example 51 | 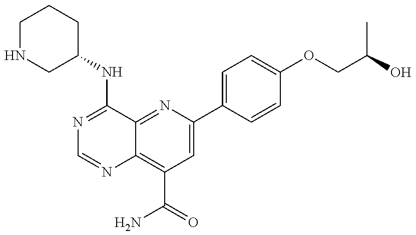 [SMILES: C[C@@H](O)COc1ccc(-c2cc(C(N)=O)c3ncnc(N[C@H]4CCCNC4)c3n2)cc1] |
| Example 52 | 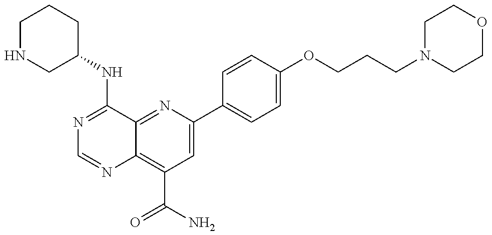 [SMILES: NC(=O)c1cc(-c2ccc(OCCCN3CCOCC3)cc2)nc2c(N[C@H]3CCCNC3)ncnc12] |
| Example 53 | 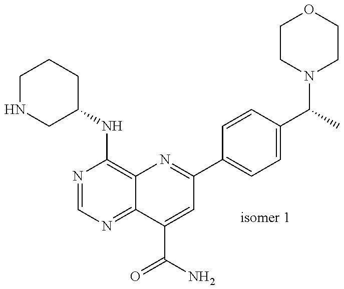 [SMILES: C[C@H](c1ccc(-c2cc(C(N)=O)c3ncnc(N[C@H]4CCCNC4)c3n2)cc1)N1CCOCC1] |
| | 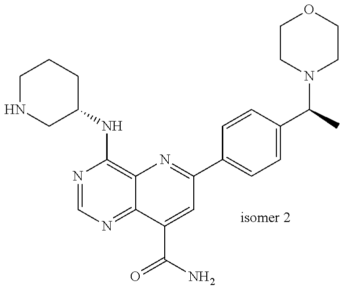 [SMILES: C[C@@H](c1ccc(-c2cc(C(N)=O)c3ncnc(N[C@H]4CCCNC4)c3n2)cc1)N1CCOCC1] |
| Example 54 | 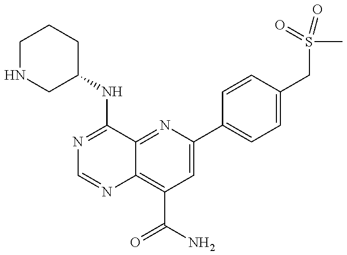 [SMILES: CS(=O)(=O)Cc1ccc(-c2cc(C(N)=O)c3ncnc(N[C@H]4CCCNC4)c3n2)cc1] |
| Example 55 | 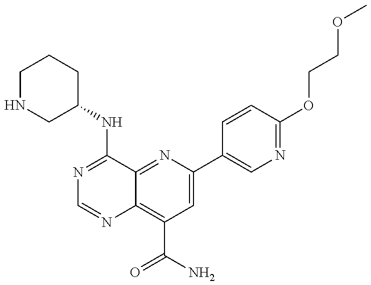 [SMILES: COCCOc1ccc(-c2cc(C(N)=O)c3ncnc(N[C@H]4CCCNC4)c3n2)cn1] |
| Example 56 | 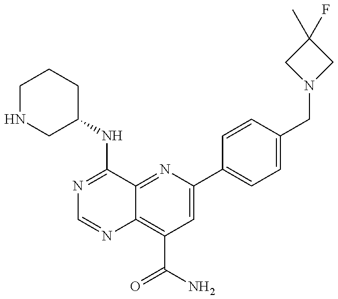 [SMILES: CC1(F)CN(Cc2ccc(-c3cc(C(N)=O)c4ncnc(N[C@H]5CCCNC5)c4n3)cc2)C1] |
| Example 57 | 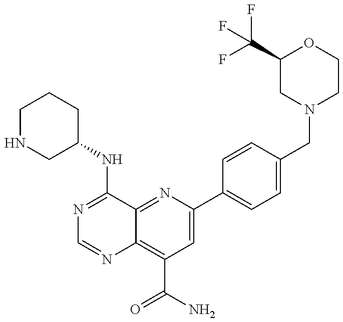 [SMILES: NC(=O)c1cc(-c2ccc(CN3CCO[C@H](C(F)(F)F)C3)cc2)nc2c(N[C@H]3CCCNC3)ncnc12] |
| Example 58 | 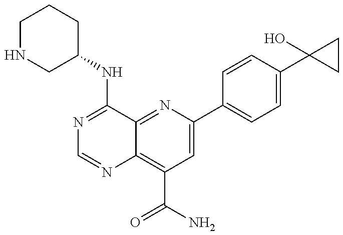 [SMILES: NC(=O)c1cc(-c2ccc(C3(O)CC3)cc2)nc2c(N[C@H]3CCCNC3)ncnc12] |
| Example 59 | 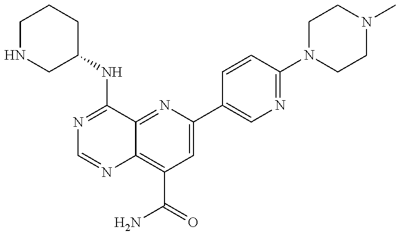 [SMILES: CN1CCN(c2ccc(-c3cc(C(N)=O)c4ncnc(N[C@H]5CCCNC5)c4n3)cn2)CC1] |
| Example 60 | 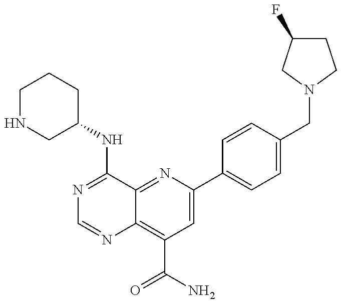 [SMILES: NC(=O)c1cc(-c2ccc(CN3CC[C@H](F)C3)cc2)nc2c(N[C@H]3CCCNC3)ncnc12] |
| Example 61 | 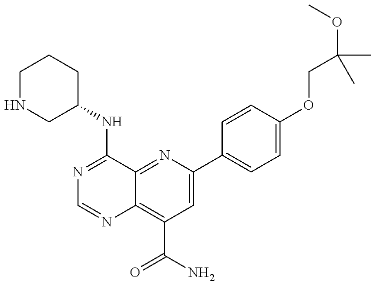 [SMILES: COC(C)(C)COc1ccc(-c2cc(C(N)=O)c3ncnc(N[C@H]4CCCNC4)c3n2)cc1] |
| Example 62 | 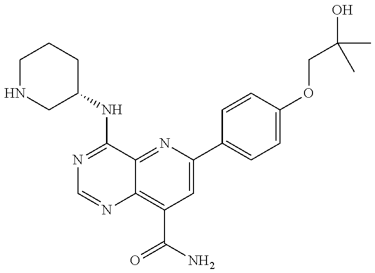 [SMILES: CC(C)(O)COc1ccc(-c2cc(C(N)=O)c3ncnc(N[C@H]4CCCNC4)c3n2)cc1] |
| Example 63 | 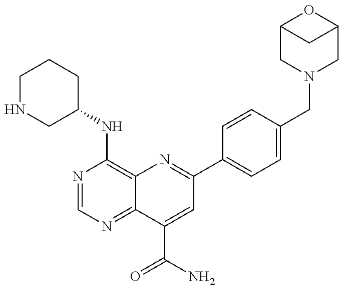 [SMILES: NC(=O)c1cc(-c2ccc(CN3CC4CC(C3)O4)cc2)nc2c(N[C@H]3CCCNC3)ncnc12] |
| Example 64 | 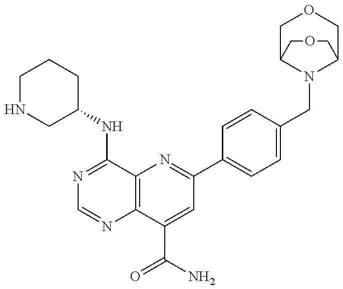 [SMILES: NC(=O)c1cc(-c2ccc(CN3C4COCC3COC4)cc2)nc2c(N[C@H]3CCCNC3)ncnc12] |
| Example 65 | 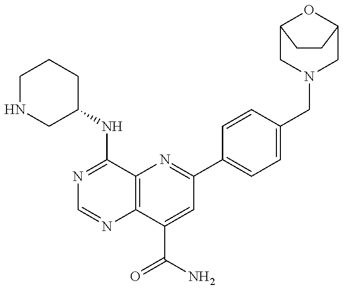 [SMILES: NC(=O)c1cc(-c2ccc(CN3CC4CCC(C3)O4)cc2)nc2c(N[C@H]3CCCNC3)ncnc12] |
| Example 66 | 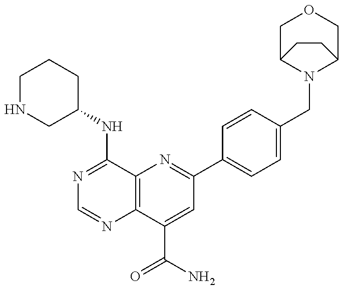 [SMILES: NC(=O)c1cc(-c2ccc(CN3C4CCC3COC4)cc2)nc2c(N[C@H]3CCCNC3)ncnc12] |
| Example 67 | 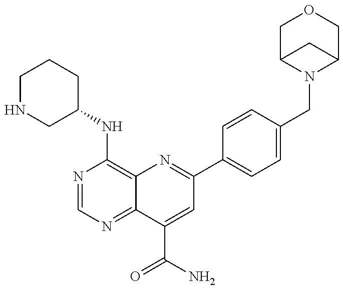 [SMILES: NC(=O)c1cc(-c2ccc(CN3C4COCC3C4)cc2)nc2c(N[C@H]3CCCNC3)ncnc12] |
| Example 68 | 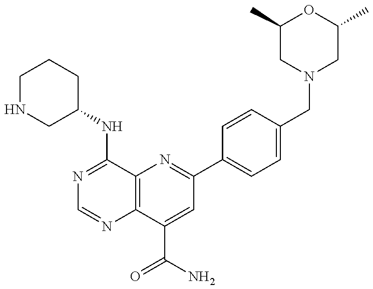 [SMILES: C[C@@H]1CN(Cc2ccc(-c3cc(C(N)=O)c4ncnc(N[C@H]5CCCNC5)c4n3)cc2)C[C@@H](C)O1] |
| Example 69 | 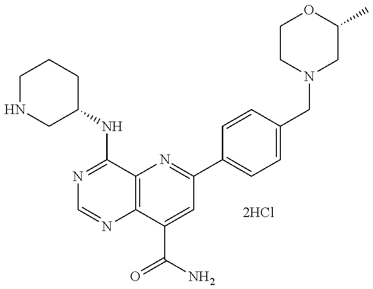 [SMILES: C[C@@H]1CN(Cc2ccc(-c3cc(C(N)=O)c4ncnc(N[C@H]5CCCNC5)c4n3)cc2)CCO1] |
| Example 70 | 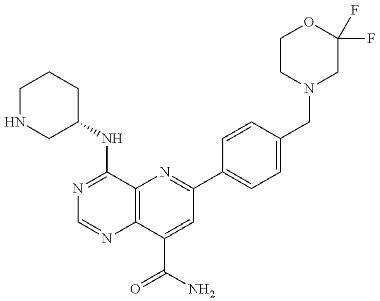 [SMILES: NC(=O)c1cc(-c2ccc(CN3CCOC(F)(F)C3)cc2)nc2c(N[C@H]3CCCNC3)ncnc12] |
| Example 71 | 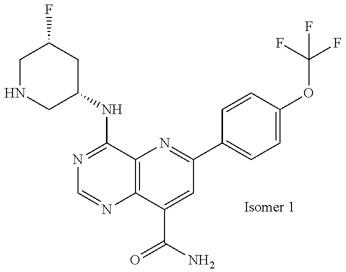 [SMILES: NC(=O)c1cc(-c2ccc(OC(F)(F)F)cc2)nc2c(N[C@@H]3CNC[C@H](F)C3)ncnc12] |
| | cis assumed; |
| | abs assumed |
| | 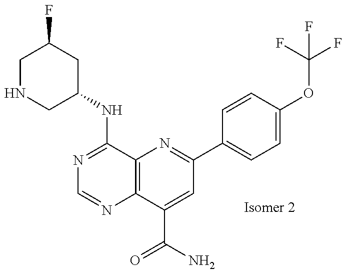 [SMILES: NC(=O)c1cc(-c2ccc(OC(F)(F)F)cc2)nc2c(N[C@@H]3CNC[C@@H](F)C3)ncnc12] |
| | trans assumed; |
| | abs assumed |
| | 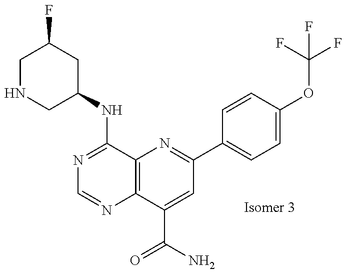 [SMILES: NC(=O)c1cc(-c2ccc(OC(F)(F)F)cc2)nc2c(N[C@H]3CNC[C@@H](F)C3)ncnc12] |
| | cis assumed; |
| | abs assumed |
| | 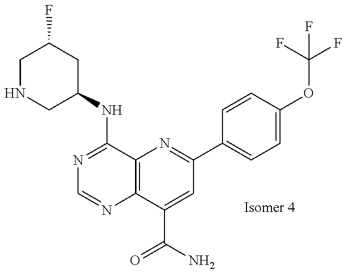 [SMILES: NC(=O)c1cc(-c2ccc(OC(F)(F)F)cc2)nc2c(N[C@H]3CNC[C@H](F)C3)ncnc12] |
| | trans assumed; |
| | abs assumed |
| Example 72 | 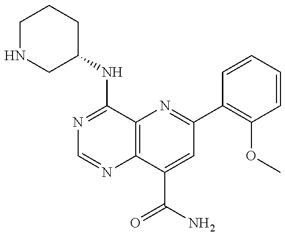 [SMILES: COc1ccccc1-c1cc(C(N)=O)c2ncnc(N[C@H]3CCCNC3)c2n1] |
| Example 76 | 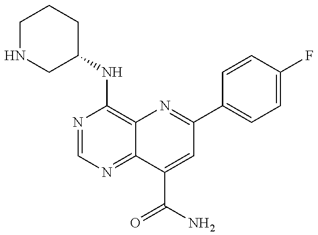 [SMILES: NC(=O)c1cc(-c2ccc(F)cc2)nc2c(N[C@H]3CCCNC3)ncnc12] |
| Example 82 | 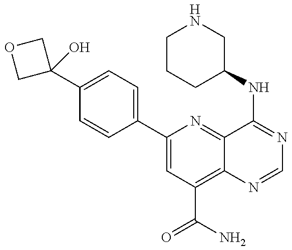 [SMILES: NC(=O)c1cc(-c2ccc(C3(O)COC3)cc2)nc2c(N[C@H]3CCCNC3)ncnc12] |
| Example 86 | 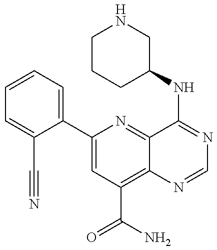 [SMILES: N#Cc1ccccc1-c1cc(C(N)=O)c2ncnc(N[C@H]3CCCNC3)c2n1] |
| Example 87 | 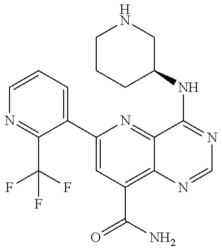 [SMILES: NC(=O)c1cc(-c2cccnc2C(F)(F)F)nc2c(N[C@H]3CCCNC3)ncnc12] |

or a prodrug or pharmaceutically acceptable salt thereof.

64. (canceled)

65. A pharmaceutical composition comprising a compound of claim 1, and a pharmaceutically acceptable adjuvant, carrier, or vehicle.

66. A method, comprising administering to a patient having an HPK1-mediated disorder a therapeutically effective amount of the compound of claim 1, or a pharmaceutically acceptable salt thereof.

67. The method of claim 66, wherein the HPK1-mediated disorder is a cancer.

68. The method of claim 67, wherein the cancer is selected from the group consisting of cancer of the breast, bladder, bone, brain, central and peripheral nervous system, colon, endocrine glands, esophagus, endometrium, germ cells, head and neck, kidney, liver, lung, larynx and hypopharynx, ovary, pancreas, prostate, rectum, renal, small intestine, soft tissue, testis, stomach, skin, ureter, vagina, and vulva.

69. The method of claim 66, wherein the therapeutically effective amount of the compound is selected from a range consisting of 0.1 to 100 mg/kg of body weight of the patient, 0.1 to 50 mg/kg of body weight of the patient, 0.5 to 50 mg/kg of body weight of the patient, 1 to 20 mg/kg of body weight of the patient, 5 to 20 mg/kg of body weight of the patient, 10 to 20 mg/kg of body weight of the patient, 10 to 50 mg/kg of body weight of the patient, and 10 to 100 mg/kg of body weight of the patient.

70. The method of claim 66, wherein the compound is administered to the patient continuously, multiple times daily, once daily, once every other day, weekly, bi-weekly, monthly, or bi-monthly.

71. The method of claim 66, wherein the compound is administered orally, parenterally, by inhalation spray, topically, rectally, nasally, buccally, vaginally, or via an implanted reservoir.

72. The method of claim 66, wherein the compound is administered subcutaneously, intravenously, intramuscularly, intra-articularly, intra-synovially, intrasternally, intrathecally, intrahepaticly, intralesionally, and by intracranial injection or infusion technique.

73. A method, comprising administering to a patient having an HPK1-mediated disorder a therapeutically effective amount of the pharmaceutical composition of claim 65 or a pharmaceutically acceptable salt thereof.

74. The method of claim 73, wherein the HPK1-mediated disorder is a cancer.

75. The method of claim 74, wherein the cancer is selected from the group consisting of cancer of the breast, bladder, bone, brain, central and peripheral nervous system, colon, endocrine glands, esophagus, endometrium, germ cells, head and neck, kidney, liver, lung, larynx and hypopharynx, ovary, pancreas, prostate, rectum, renal, small intestine, soft tissue, testis, stomach, skin, ureter, vagina, and vulva.

76. The method of claim 73, wherein the therapeutically effective amount of the pharmaceutical composition is selected from a range consisting of 0.1 to 100 mg/kg of body weight of the patient, 0.1 to 50 mg/kg of body weight of the patient, 0.5 to 50 mg/kg of body weight of the patient, 1 to 20 mg/kg of body weight of the patient, 5 to 20 mg/kg of body weight of the patient, 10 to 20 mg/kg of body weight of the patient, 10 to 50 mg/kg of body weight of the patient, and 10 to 100 mg/kg of body weight of the patient.

77. The method of claim 76, wherein the pharmaceutical composition is administered to the patient continuously, multiple times daily, once daily, once every other day, weekly, bi-weekly, monthly, or bi-monthly.

78. The method of claim 76, wherein the compound is administered subcutaneously, intravenously, intramuscularly, intra-articularly, intra-synovially, intrasternally, intrathecally, intrahepaticly, intralesionally, and by intracranial injection or infusion technique.

79. (canceled)
